SEQUENCE LISTING (346 sequences)

SEQ ID NO: 1 - protein, 6 aa, synthetic construct
GDYAWN

SEQ ID NO: 2 - protein, 16 aa, synthetic construct
YISYSGSTSYNPSLQS

SEQ ID NO: 3 - protein, 10 aa, synthetic construct
SLGRRWYFVV

SEQ ID NO: 4 - protein, 17 aa, synthetic construct
KSSQSLLNSSNQKNYLA

SEQ ID NO: 5 - protein, 7 aa, synthetic construct
FASTRES

SEQ ID NO: 6 - protein, 9 aa, synthetic construct
QQHYSAPWT

SEQ ID NO: 7 - protein, 9 aa, synthetic construct
DYSITGDYA

SEQ ID NO: 8 - protein, 7 aa, synthetic construct
ISYSGST

SEQ ID NO: 9 - protein, 12 aa, synthetic construct
ARSLGRRWYFVV

SEQ ID NO: 10 - protein, 12 aa, synthetic construct
QSLLNSSNQKNY

SEQ ID NO: 11 - skipped

SEQ ID NO: 12 - protein, 9 aa, synthetic construct
QQHYSAPWT

SEQ ID NO: 13 - protein, 119 aa, synthetic construct
DVQLQESGPGLVKPSQSLSLTCTVTDYSITGDYAWNWIRQFPGNKLEWMGYISYSGSTSYNPSLQSRISITRDTSKNQFFLQLNSVTSEDTATYFCARSLGRRWYFVVWGAGTTVTVSA

SEQ ID NO: 14 - protein, 113 aa, synthetic construct
DIVMTQSPSSLAMSVGQKVTMSCKSSQSLLNSSNQKNYLAWYQQKPGQSPKLLIYFASTRESGVPDRFIGSGSGTDFTLTISSVQAEDLTDYFCQQHYSAPWTFGGGTKLEIK

SEQ ID NO: 15 - protein, 448 aa, synthetic construct
DVQLQESGPGLVKPSQSLSLTCTVTDYSITGDYAWNWIRQFPGNKLEWMGYISYSGSTSYNPSLQSRISITRDTSKNQFFLQLNSVTSEDTATYFCARSLGRRWYFVVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 220 aa, synthetic construct
DIVMTQSPSSLAMSVGQKVTMSCKSSQSLLNSSNQKNYLAWYQQKPGQSPKLLIYFASTRESGVPDRFIGSGSGTDFTLTISSVQAEDLTDYFCQQHYSAPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 6 aa, synthetic construct
GDYAWN

SEQ ID NO: 18 - protein, 16 aa, synthetic construct
YISYSGSTSYNPSLQS

SEQ ID NO: 19 - protein, 10 aa, synthetic construct
SLGRRWYFVV

SEQ ID NO: 20 - protein, 17 aa, synthetic construct
KSSQSLLQSSNQKNYLA

SEQ ID NO: 21 - protein, 7 aa, synthetic construct
FASTRES

SEQ ID NO: 22 - protein, 9 aa, synthetic construct
QQHYSAPWT

SEQ ID NO: 23 - protein, 9 aa, synthetic construct
DYSITGDYA

SEQ ID NO: 24 - protein, 7 aa, synthetic construct
ISYSGST

SEQ ID NO: 25 - protein, 12 aa, synthetic construct
ARSLGRRWYFVV

SEQ ID NO: 26 - protein, 12 aa, synthetic construct
QSLLQSSNQKNY

SEQ ID NO: 27 - skipped

SEQ ID NO: 28 - protein, 9 aa, synthetic construct
QQHYSAPWT

SEQ ID NO: 29 - protein, 119 aa, synthetic construct
QVQLQESGPGLVKPSQTLSLTCTVTDYSITGDYAWNWIRQHPGKGLEWIGYISYSGSTSYNPSLQSRVTISRDTSKNQFSLKLSSVTAADTAVYFCARSLGRRWYFVVWGQGTTVTVSS

SEQ ID NO: 30 - protein, 113 aa, synthetic construct
DIVMTQSPDSLAVSLGERATINCKSSQSLLQSSNQKNYLAWYQQKPGQPPKLLIYFASTRESGVPDRFSGSGSGTDFTLTISSLQAEDVAVYYCQQHYSAPWTFGGGTKVEIK

SEQ ID NO: 31 - protein, 449 aa, synthetic construct
QVQLQESGPGLVKPSQTLSLTCTVTDYSITGDYAWNWIRQHPGKGLEWIGYISYSGSTSYNPSLQSRVTISRDTSKNQFSLKLSSVTAADTAVYFCARSLGRRWYFVVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 220 aa, synthetic construct
DIVMTQSPDSLAVSLGERATINCKSSQSLLQSSNQKNYLAWYQQKPGQPPKLLIYFASTRESGVPDRFSGSGSGTDFTLTISSLQAEDVAVYYCQQHYSAPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 5 aa, synthetic construct
NYGMN

SEQ ID NO: 34 - protein, 17 aa, synthetic construct
WINTYTGEPTYTDDFKG

SEQ ID NO: 35 - protein, 12 aa, synthetic construct
GGFGSSYWYFDV

SEQ ID NO: 36 - protein, 11 aa, synthetic construct
KASQDVSIAVA

SEQ ID NO: 37 - protein, 7 aa, synthetic construct
SASYRYT

SEQ ID NO: 38 - protein, 9 aa, synthetic construct
QQHYITPLT

SEQ ID NO: 39 - protein, 8 aa, synthetic construct
GYTFTNYG

SEQ ID NO: 40 - protein, 8 aa, synthetic construct
INTYTGEP

SEQ ID NO: 41 - protein, 14 aa, synthetic construct
ARGGFGSSYWYFDV

SEQ ID NO: 42 - protein, 6 aa, synthetic construct
QDVSIA

SEQ ID NO: 43 - skipped

SEQ ID NO: 44 - protein, 9 aa, synthetic construct
QQHYITPLT

SEQ ID NO: 45 - protein, 121 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 46 - protein, 107 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 47 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 5 aa, synthetic construct
DTYIH

SEQ ID NO: 50 - protein, 17 aa, synthetic construct
RIYPTNGYTRYADSVKG

SEQ ID NO: 51 - protein, 11 aa, synthetic construct
WGGDGFYAMDY

SEQ ID NO: 52 - protein, 11 aa, synthetic construct
RASQDVNTAVA

SEQ ID NO: 53 - protein, 7 aa, synthetic construct
SASFLYS

SEQ ID NO: 54 - protein, 9 aa, synthetic construct
QQHYTTPPT

SEQ ID NO: 55 - protein, 8 aa, synthetic construct
GFNIKDTY

SEQ ID NO: 56 - protein, 8 aa, synthetic construct
IYPTNGYT

SEQ ID NO: 57 - protein, 13 aa, synthetic construct
SRWGGDGFYAMDY

SEQ ID NO: 58 - protein, 6 aa, synthetic construct
QDVNTA

SEQ ID NO: 59 - skipped

SEQ ID NO: 60 - protein, 9 aa, synthetic construct
QQHYTTPPT

SEQ ID NO: 61 - protein, 120 aa, synthetic construct [sequence omitted]

SEQ ID NO: 62 - protein, 107 aa, synthetic construct [sequence omitted]

SEQ ID NO: 63 - protein, 449 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 64 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 5 aa, synthetic construct
DYTMD

SEQ ID NO: 66 - protein, 17 aa, synthetic construct
DVNPNSGGSIYNQRFKG

SEQ ID NO: 67 - protein, 10 aa, synthetic construct
NLGPSFYFDY

SEQ ID NO: 68 - protein, 11 aa, synthetic construct
KASQDVSIGVA

SEQ ID NO: 69 - protein, 7 aa, synthetic construct
SASYRYT

SEQ ID NO: 70 - protein, 9 aa, synthetic construct
QQYYIYPYT

SEQ ID NO: 71 - protein, 8 aa, synthetic construct
GFTFTDYT

SEQ ID NO: 72 - protein, 8 aa, synthetic construct
VNPNSGGS

SEQ ID NO: 73 - protein, 12 aa, synthetic construct
ARNLGPSFYFDY

SEQ ID NO: 74 - protein, 6 aa, synthetic construct
QDVSIG

SEQ ID NO: 75 - skipped

SEQ ID NO: 76 - protein, 9 aa, synthetic construct
QQYYIYPYT

SEQ ID NO: 77 - protein, 119 aa, synthetic construct [sequence omitted]

SEQ ID NO: 78 - protein, 107 aa, synthetic construct [sequence omitted]

SEQ ID NO: 79 - protein, 448 aa, synthetic construct
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 80 - protein, 214 aa, synthetic construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 5 aa, synthetic construct
SYWMY

SEQ ID NO: 82 - protein, 17 aa, synthetic construct
SINSDSSSTYYRDSVKG

SEQ ID NO: 83 - protein, 6 aa, synthetic construct
DPGGYA

SEQ ID NO: 84 - protein, 8 aa, synthetic construct
GFTFSSYW

SEQ ID NO: 85 - protein, 8 aa, synthetic construct
INSDSSST

SEQ ID NO: 86 - protein, 8 aa, synthetic construct
AKDPGGYA

SEQ ID NO: 87 - protein, 115 aa, synthetic construct
EVQLQESGGGLVQPGGSLRLSCAASGFTFSSYWMYWLRQAPGKGLEWVSSINSDSSSTYYRDSVKGRFTISRDNAKNTLYLQMNSLKSEDTAVYYCAKDPGGYAKGQGTQVTVSS

SEQ ID NO: 88 - protein, 5 aa, synthetic construct
NYGMN

SEQ ID NO: 89 - protein, 17 aa, synthetic construct
WINTYTGEPTYAADFKR

SEQ ID NO: 90 - protein, 14 aa, synthetic construct
YPHYYGSSHWYFDV

SEQ ID NO: 91 - protein, 11 aa, synthetic construct
SASQDISNYLN

SEQ ID NO: 92 - protein, 7 aa, synthetic construct
FTSSLHS

SEQ ID NO: 93 - protein, 9 aa, synthetic construct
QQYSTVPWT

SEQ ID NO: 94 - protein, 8 aa, synthetic construct
GYTFTNYG

SEQ ID NO: 95 - protein, 8 aa, synthetic construct
INTYTGEP

SEQ ID NO: 96 - protein, 16 aa, synthetic construct
AKYPHYYGSSHWYFDV

SEQ ID NO: 97 - protein, 6 aa, synthetic construct
QDISNY

SEQ ID NO: 98 - skipped

SEQ ID NO: 99 - protein, 9 aa, synthetic construct
QQYSTVPWT

SEQ ID NO: 100 - protein, 123 aa, synthetic construct [sequence omitted]

SEQ ID NO: 101 - protein, 107 aa, synthetic construct [sequence omitted]

SEQ ID NO: 102 - protein, 103 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 103 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 104 - protein, 107 aa, synthetic construct [sequence omitted]

SEQ ID NO: 105 - protein, 452 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 106 - protein, 214 aa, synthetic construct
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - protein, 11 aa, synthetic construct
GGGGSGGGGSG

SEQ ID NO: 108 - protein, 578 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGEVQLQESGGGLVQPGGSLRLSCAASGFTFSSYWMYWLRQAPGKGLEWVSSINSDSSSTYYRDSVKGRFTISRDNAKNTLYLQMNSLKSEDTAVYYCAKDPGGYAKGQGTQVTVSS

SEQ ID NO: 109 - protein, 214 aa, synthetic construct; identical to SEQ ID NO: 106

SEQ ID NO: 110 - protein, 5 aa, synthetic construct
RNFMG

SEQ ID NO: 111 - protein, 17 aa, synthetic construct
AIYTGTSRTYYADSVKG

SEQ ID NO: 112 - protein, 14 aa, synthetic construct
DLRDGFWDTGVWNT

SEQ ID NO: 113 - protein, 14 aa, synthetic construct
DLREGFWDTGVWNT

SEQ ID NO: 114 - protein, 8 aa, synthetic construct
GNIYNRNF

SEQ ID NO: 115 - protein, 8 aa, synthetic construct
IYTGTSRT

SEQ ID NO: 116 - protein, 16 aa, synthetic construct
AADLRDGFWDTGVWNT

SEQ ID NO: 117 - protein, 16 aa, synthetic construct
AADLREGFWDTGVWNT

SEQ ID NO: 118 - protein, 123 aa, synthetic construct
EVQLVESGGGLVQPGGSLRLSCAVSGNIYNRNFMGWFRQAPGKGREGVSAIYTGTSRTYYADSVKGRFTISRDNAKNTVYLQMNSLRPEDTAVYYCAADLRDGFWDTGVWNTWGQGTLVTVSS

SEQ ID NO: 119 - protein, 123 aa, synthetic construct
EVQLVESGGGLVQPGGSLRLSCAVSGNIYNRNFMGWFRQAPGKGLEGVSAIYTGTSRTYYADSVKGRFTISRDNSKNTVYLQMNSLRAEDTAVYYCAADLREGFWDTGVWNTWGQGTLVTVSS

SEQ ID NO: 120 - protein, 5 aa, synthetic construct
SYWMY

SEQ ID NO: 121 - protein, 17 aa, synthetic construct
SINSDSSSTYYRDSVKG

SEQ ID NO: 122 - protein, 6 aa, synthetic construct
DPGGYA

SEQ ID NO: 123 - protein, 8 aa, synthetic construct
GFTFSSYW

SEQ ID NO: 124 - protein, 8 aa, synthetic construct
INSDSSST

SEQ ID NO: 125 - protein, 8 aa, synthetic construct
AKDPGGYA

SEQ ID NO: 126 - protein, 115 aa, synthetic construct; identical to SEQ ID NO: 87

SEQ ID NO: 127 - protein, 8 aa, synthetic construct
GYAFTGYT

SEQ ID NO: 128 - protein, 8 aa, synthetic construct
FYPGSGTL

SEQ ID NO: 129 - protein, 13 aa, synthetic construct
ARHGTGTLMAMDY

SEQ ID NO: 130 - protein, 11 aa, synthetic construct
QSLANSYGNTY

SEQ ID NO: 131 - skipped

SEQ ID NO: 132 - protein, 9 aa, synthetic construct
LQGTHQPPT

SEQ ID NO: 133 - protein, 120 aa, synthetic construct
QVQLVQSGAEVKKPGASVKVSCKASGYAFTGYTIHWVRQAPGQRLEWMGWFYPGSGTLKYSEKFQGRVTITRDKSLSTAYMELSSLRSEDTAVYYCARHGTGTLMAMDYWGQGTLVTVSS

SEQ ID NO: 134 - protein, 112 aa, synthetic construct
DVVMTQTPLSLSVTPGQPASISCKSSQSLANSYGNTYLSWYLHKPGQSPQLLIYGISNRFSGVPDRFSGSGSGTDFTLKISRVEAEDVGVYYCLQGTHQPPTFGQGTKLEIK

SEQ ID NO: 135 - protein, 5 aa, synthetic construct
NYGMN

SEQ ID NO: 136 - protein, 17 aa, synthetic construct
WINTYTGEPTYAADFKR

SEQ ID NO: 137 - protein, 14 aa, synthetic construct
YPHYYGSSHWYFDV

SEQ ID NO: 138 - protein, 11 aa, synthetic construct
SASQDISNYLN

SEQ ID NO: 139 - protein, 7 aa, synthetic construct
FTSSLHS

SEQ ID NO: 140 - protein, 9 aa, synthetic construct
QQYSTVPWT

SEQ ID NO: 141 - protein, 8 aa, synthetic construct
GYTFTNYG

SEQ ID NO: 142 - protein, 8 aa, synthetic construct
INTYTGEP

SEQ ID NO: 143 - protein, 16 aa, synthetic construct
AKYPHYYGSSHWYFDV

SEQ ID NO: 144 - protein, 6 aa, synthetic construct
QDISNY

SEQ ID NO: 145 - skipped

SEQ ID NO: 146 - protein, 123 aa, synthetic construct [sequence omitted]

SEQ ID NO: 147 - protein, 107 aa, synthetic construct [sequence omitted]

SEQ ID NO: 148 - protein, 103 aa, synthetic construct; identical to SEQ ID NO: 102

SEQ ID NO: 149 - protein, 226 aa, synthetic construct; identical to SEQ ID NO: 103

SEQ ID NO: 150 - protein, 107 aa, synthetic construct [sequence omitted]

SEQ ID NO: 151 - protein, 452 aa, synthetic construct; identical to SEQ ID NO: 105

SEQ ID NO: 152 - protein, 214 aa, synthetic construct; identical to SEQ ID NO: 106

SEQ ID NO: 153 - protein, 102 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTII

SEQ ID NO: 154 - protein, 103 aa, synthetic construct
DVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 155 - protein, 100 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNT

SEQ ID NO: 156 - protein, 205 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 157 - protein, 432 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 578 aa, synthetic construct; identical to SEQ ID NO: 108

SEQ ID NO: 159 - protein, 719 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGFAFSSYDMSWVRQAPGKGLDWVATISGGGRYTYYPDSVKGRFTISRDNSKNNLYLQMNSLRAEDTALYYCANRYGEAWFAYWGQGTLVTVSSGGGGSGGGGSGGGGSGGGGSDIQMTQSPSSMSASVGDRVTFTCRASQDINTYLSWFQQKPGKSPKTLIYRANRLVSGVPSRFSGSGSGQDYTLTISSLQPEDMATYYCLQYDEFPLTFGAGTKLELKR

SEQ ID NO: 160 - protein, 739 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAVSGNIYNRNFMGWFRQAPGKGREGVSAIYTGTSRTYYADSVKGRFTISRDNAKNTVYLQMNSLRPEDTAVYYCAADLRDGFWDTGVWNTWGQGTLVTVSSGGGGSGGGGSGGGGSGGGGSEVQLVESGGGLVQPGGSLRLSCAVSGNIYNRNFMGWFRQAPGKGREGVSAIYTGTSRTYYADSVKGRFTISRDNAKNTVYLQMNSLRPEDTAVYYCAADLRDGFWDTGVWNTWGQGTLVTVSS

SEQ ID NO: 161 - protein, 595 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLVESGGGLVQPGGSLRLSCAVSGNIYNRNFMGWFRQAPGKGREGVSAIYTGTSRTYYADSVKGRFTISRDNAKNTVYLQMNSLRPEDTAVYYCAADLRDGFWDTGVWNTWGQGTLVTVSS

SEQ ID NO: 162 - protein, 595 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLVESGGGLVQPGGSLRLSCAVSGNIYNRNFMGWFRQAPGKGLEGVSAIYTGTSRTYYADSVKGRFTISRDNSKNTVYLQMNSLRAEDTAVYYCAADLREGFWDTGVWNTWGQGTLVTVSS

SEQ ID NO: 163 - protein, 564 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTGGSGGSGGSGGSGGSQVQLVQSGAEVKKPGASVKVSCKASGYAFTGYTIHWVRQAPGQRLEWMGWFYPGSGTLKYSEKFQGRVTITRDKSLSTAYMELSSLRSEDTAVYYCARHGTGTLMAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 164 - protein, 219 aa, synthetic construct
DVVMTQTPLSLSVTPGQPASISCKSSQSLANSYGNTYLSWYLHKPGQSPQLLIYGISNRFSGVPDRFSGSGSGTDFTLKISRVEAEDVGVYYCLQGTHQPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 165 - protein, 564 aa, synthetic construct
QVQLVQSGAEVKKPGASVKVSCKASGYAFTGYTIHWVRQAPGQRLEWMGWFYPGSGTLKYSEKFQGRVTITRDKSLSTAYMELSSLRSEDTAVYYCARHGTGTLMAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGSGGSGGSGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNT

SEQ ID NO: 166 - protein, 11 aa, synthetic construct
GGGGSGGGGSG

SEQ ID NO: 167 - protein, 20 aa, synthetic construct
GGGGSGGGGSGGGGSGGGGS

SEQ ID NO: 168 - protein, 15 aa, synthetic construct
GGSGGSGGSGGSGGS

SEQ ID NO: 169 - protein, 288 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 170 - protein, 288 aa, synthetic construct
MQIPQAPWPVVWAVLQLGWRPGWFLDSPDRPWNPPTFFPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 171 - protein, 288 aa, synthetic construct [sequence omitted]

SEQ ID NO: 172 - protein, 290 aa, synthetic construct
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 173 - protein, 176 aa, synthetic construct
MRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 174 - protein, 176 aa, synthetic construct; identical to SEQ ID NO: 173

SEQ ID NO: 175 - protein, 178 aa, synthetic construct
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGD

SEQ ID NO: 176 - protein, 22 aa, synthetic construct
MNFLLSWVHWSLALLLYLHHAK

SEQ ID NO: 177 - protein, 395 aa, synthetic construct
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 178 - protein, 8 aa, synthetic construct
GFAFSSYD

SEQ ID NO: 179 - protein, 8 aa, synthetic construct
ISGGGRYT

SEQ ID NO: 180 - protein, 11 aa, synthetic construct
ANRYGEAWFAY

SEQ ID NO: 181 - protein, 6 aa, synthetic construct
QDINTY

SEQ ID NO: 182 - skipped

SEQ ID NO: 183 - protein, 9 aa, synthetic construct
LQYDEFPLT

SEQ ID NO: 184 - protein, 5 aa, synthetic construct
SYDMS

SEQ ID NO: 185 - protein, 17 aa, synthetic construct
TISGGGRYTYYPDSVKG

SEQ ID NO: 186 - protein, 9 aa, synthetic construct
RYGEAWFAY

SEQ ID NO: 187 - protein, 11 aa, synthetic construct
RASQDINTYLS

SEQ ID NO: 188 - protein, 7 aa, synthetic construct
RANRLVS

SEQ ID NO: 189 - protein, 246 aa, synthetic construct
EVQLVESGGGLVQPGGSLRLSCAASGFAFSSYDMSWVRQAPGKGLDWVATISGGGRYTYYPDSVKGRFTISRDNSKNNLYLQMNSLRAEDTALYYCANRYGEAWFAYWGQGTLVTVSSGGGGSGGGGSGGGGSGGGGSDIQMTQSPSSMSASVGDRVTFTCRASQDINTYLSWFQQKPGKSPKTLIYRANRLVSGVPSRFSGSGSGQDYTLTISSLQPEDMATYYCLQYDEFPLTFGAGTKLELKR

SEQ ID NO: 190 - protein, 5 aa, synthetic construct
TYGMH

SEQ ID NO: 191 - protein, 17 aa, synthetic construct
VIWYDVSHKYYADSVKG

SEQ ID NO: 192 - protein, 10 aa, synthetic construct
DWGDPDAFDI

SEQ ID NO: 193 - protein, 11 aa, synthetic construct
RASQGISSYLA

SEQ ID NO: 194 - protein, 7 aa, synthetic construct
AASTLQS

SEQ ID NO: 195 - protein, 9 aa, synthetic construct
QQLNSYPLT

SEQ ID NO: 196 - protein, 8 aa, synthetic construct
GFTFSTYG

SEQ ID NO: 197 - protein, 8 aa, synthetic construct
IWYDVSHK

SEQ ID NO: 198 - protein, 12 aa, synthetic construct
ARDWGDPDAFDI

SEQ ID NO: 199 - protein, 6 aa, synthetic construct
QGISSY

SEQ ID NO: 200 - skipped

SEQ ID NO: 201 - protein, 9 aa, synthetic construct
QQLNSYPLT

SEQ ID NO: 202 - protein, 119 aa, synthetic construct
EVQLVESGGGVVQPGRSLRLSCAASGFTFSTYGMHWVRQAPGKGLEWVAVIWYDVSHKYYADSVKGRFTISRDNSKNTLSLQMNSLRAEDTAVYYCARDWGDPDAFDIWGQGTMVTVSS

SEQ ID NO: 203 - protein, 107 aa, synthetic construct
DIQLTQSPSFLSASVGDRVTITCRASQGISSYLAWYQQKPGKAPKLLIYAASTLQSGVPSRFSGSGSGTEFTLTISSLQPEDFATYYCQQLNSYPLTFGGGTKVEIK

SEQ ID NO: 204 - protein, 449 aa, synthetic construct
EVQLVESGGGVVQPGRSLRLSCAASGFTFSTYGMHWVRQAPGKGLEWVAVIWYDVSHKYYADSVKGRFTISRDNSKNTLSLQMNSLRAEDTAVYYCARDWGDPDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 205 - protein, 214 aa, synthetic construct
DIQLTQSPSFLSASVGDRVTITCRASQGISSYLAWYQQKPGKAPKLLIYAASTLQSGVPSRFSGSGSGTEFTLTISSLQPEDFATYYCQQLNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 206 - protein, 5 aa, synthetic construct
GYFWS

SEQ ID NO: 207 - protein, 16 aa, synthetic construct
YIHYSGRTNYNPSLKS

SEQ ID NO: 208 - protein, 12 aa, synthetic construct
ETGGSGHWYFDL

SEQ ID NO: 209 - protein, 11 aa, synthetic construct
RASQSISSYLI

SEQ ID NO: 210 - protein, 7 aa, synthetic construct
AASSLQS

SEQ ID NO: 211 - protein, 9 aa, synthetic construct
HQSYSTPWT

SEQ ID NO: 212 - protein, 8 aa, synthetic construct
GGSISGYF

SEQ ID NO: 213 - protein, 7 aa, synthetic construct
IHYSGRT

SEQ ID NO: 214 - protein, 14 aa, synthetic construct
ARETGGSGHWYFDL

SEQ ID NO: 215 - protein, 6 aa, synthetic construct
QSISSY

SEQ ID NO: 216 - skipped

SEQ ID NO: 217 - protein, 9 aa, synthetic construct
HQSYSTPWT

SEQ ID NO: 218 - protein, 120 aa, synthetic construct
QVQLQESGPGLVKPSETLSLTCTVSGGSISGYFWSWIRQSPGKGLEWIGYIHYSGRTNYNPSLKSRVTISVDTSKNQFSLKLSSVTAADTAVYYCARETGGSGHWYFDLWGRGNLVTVSS

SEQ ID NO: 219 - protein, 107 aa, synthetic construct
DIQMTQSPSSLSASVGDRVTITCRASQSISSYLIWYQQKPGKAPKLLIYAASSLQSGVPSRFSGSGSGTDFTLTISSLQPEDFATYYCHQSYSTPWTFGQGTKVEIK

SEQ ID NO: 220 - protein, 5 aa, synthetic construct
GYFWS

SEQ ID NO: 221 - protein, 16 aa, synthetic construct
YIHYSGRTNYNPSLKS

SEQ ID NO: 222 - protein, 12 aa, synthetic construct
ETGGSGHWYFDL

SEQ ID NO: 223 - protein, 11 aa, synthetic construct
RASQSISSYLI

SEQ ID NO: 224 - protein, 7 aa, synthetic construct
AASSLQS

SEQ ID NO: 225 - protein, 9 aa, synthetic construct
HQSYSTPWT

SEQ ID NO: 226 - protein, 8 aa, synthetic construct
GGSISGYF

SEQ ID NO: 227 - protein, 7 aa, synthetic construct
IHYSGRT

SEQ ID NO: 228 - protein, 14 aa, synthetic construct
ARETGGSGHWYFDL

SEQ ID NO: 229 - protein, 6 aa, synthetic construct
QSISSY

SEQ ID NO: 230 - skipped

SEQ ID NO: 231 - protein, 9 aa, synthetic construct
HQSYSTPWT

SEQ ID NO: 232 - protein, 120 aa, synthetic construct
QVQLQESGPGLVKPSETLSLTCTVSGGSISGYFWSWIRQSPGQGLEWIGYIHYSGRTNYNPSLKSRVTISVDTSKNQFSLQLSSVTAADTAVYYCARETGGSGHWYFDLWGRGNLVTVSS

SEQ ID NO: 233 - protein, 107 aa, synthetic construct
DIQMTQSPSSLSASVGDRVTITCRASQSISSYLIWYQQKPGQAPRLLIYAASSLQSGVPSRFSGSGSGTDFTLTISSLQPEDFATYYCHQSYSTPWTFGQGTKVEIK

SEQ ID NO: 234 - protein, 5 aa, synthetic construct
DYYIT

SEQ ID NO: 235 - protein, 17 aa, synthetic construct
WIYPGSGNTKYNEKFKG

SEQ ID NO: 236 - protein, 8 aa, synthetic construct
YGNYWFAY

SEQ ID NO: 237 - protein, 15 aa, synthetic construct
KASQSVDFDGDSYMN

SEQ ID NO: 238 - protein, 7 aa, synthetic construct
AASNLES

SEQ ID NO: 239 - protein, 9 aa, synthetic construct
QQSNEDPWT

SEQ ID NO: 240 - protein, 8 aa, synthetic construct
GYTFTDYY

SEQ ID NO: 241 - protein, 8 aa, synthetic construct
IYPGSGNT

SEQ ID NO: 242 - protein, 10 aa, synthetic construct
ANYGNYWFAY

SEQ ID NO: 243 - protein, 10 aa, synthetic construct
QSVDFDGDSY

SEQ ID NO: 244 - skipped

SEQ ID NO: 245 - protein, 9 aa, synthetic construct
QQSNEDPWT

SEQ ID NO: 246 - protein, 117 aa, synthetic construct
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSA

SEQ ID NO: 247 - protein, 111 aa, synthetic construct
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIK

SEQ ID NO: 248 - protein, 446 aa, synthetic construct
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 249 - protein, 218 aa, synthetic construct
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 250 - protein, 5 aa, synthetic construct
SYNMN

SEQ ID NO: 251 - protein, 17 aa, synthetic construct
YISSSSSTIYYADSVKG

SEQ ID NO: 252 - protein, 8 aa, synthetic construct
AYYYGMDV

SEQ ID NO: 253 - protein, 11 aa, synthetic construct
RASQGISGWLA

SEQ ID NO: 254 - protein, 7 aa, synthetic construct
AASTLQS

SEQ ID NO: 255 - protein, 9 aa, synthetic construct
QQANSFPPT

SEQ ID NO: 256 - protein, 8 aa, synthetic construct
GFTFSSYN

SEQ ID NO: 257 - protein, 8 aa, synthetic construct
ISSSSSTI

SEQ ID NO: 258 - protein, 10 aa, synthetic construct
ARAYYYGMDV

SEQ ID NO: 259 - protein, 6 aa, synthetic construct
QGISGW

SEQ ID NO: 260 - skipped

SEQ ID NO: 261 - protein, 9 aa, synthetic construct
QQANSFPPT

SEQ ID NO: 262 - protein, 117 aa, synthetic construct
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSS

SEQ ID NO: 263 - protein, 107 aa, synthetic construct
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIK

SEQ ID NO: 264 - protein, 447 aa, synthetic construct
  VARIANT 447: note = the residue is present or absent
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 265 - protein, 214 aa, synthetic construct
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 266 - protein, 5 aa, synthetic construct
NYAMS

SEQ ID NO: 267 - protein, 17 aa, synthetic construct
SISGSGDYTYYTDSVKG

SEQ ID NO: 268 - protein, 9 aa, synthetic construct
SPWGYYLDS

SEQ ID NO: 269 - protein, 11 aa, synthetic construct
RASQGISSRLA

SEQ ID NO: 270 - protein, 7 aa, synthetic construct
AASSLQS

SEQ ID NO: 271 - protein, 9 aa, synthetic construct
QQYNSYPYT

SEQ ID NO: 272 - protein, 8 aa, synthetic construct
GFTFSNYA

SEQ ID NO: 273 - protein, 8 aa, synthetic construct
ISGSGDYT

SEQ ID NO: 274 - protein, 11 aa, synthetic construct
ARSPWGYYLDS

SEQ ID NO: 275 - protein, 6 aa, synthetic construct
QGISSR

SEQ ID NO: 276 - skipped

SEQ ID NO: 277 - protein, 9 aa, synthetic construct
QQYNSYPYT

SEQ ID NO: 278 - protein, 118 aa, synthetic construct
EVQLLESGGGLVQPGGSLRLSCAASGFTFSNYAMSWVRQAPGKGLEWVSSISGSGDYTYYTDSVKGRFTISRDNSKNTLYLQMNSLRAEDTAVYYCARSPWGYYLDSWGQGTLVTVSS

SEQ ID NO: 279 - protein, 107 aa, synthetic construct
DIQMTQSPPSLSASAGDRVTITCRASQGISSRLAWYQQKPEKAPKSLIYAASSLQSGVPSRFSGSGSGTDFTLTISSLQPEDFATYYCQQYNSYPYTFGQGTKLEIK

SEQ ID NO: 280 - protein, 448 aa, synthetic construct
EVQLLESGGGLVQPGGSLRLSCAASGFTFSNYAMSWVRQAPGKGLEWVSSISGSGDYTYYTDSVKGRFTISRDNSKNTLYLQMNSLRAEDTAVYYCARSPWGYYLDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 281 - protein, 214 aa, synthetic construct
DIQMTQSPPSLSASAGDRVTITCRASQGISSRLAWYQQKPEKAPKSLIYAASSLQSGVPSRFSGSGSGTDFTLTISSLQPEDFATYYCQQYNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 282 - protein, 7 aa, synthetic construct
SGSYYWG

SEQ ID NO: 283 - protein, 16 aa, synthetic construct
NIYYSGSTYYNPSLRS

SEQ ID NO: 284 - protein, 10 aa, synthetic construct
EGSYPNQFDP

SEQ ID NO: 285 - protein, 11 aa, synthetic construct
RASQSVSSNLA

SEQ ID NO: 286 - protein, 7 aa, synthetic construct
GASTRAT

SEQ ID NO: 287 - protein, 9 aa, synthetic construct
QQYHSFPFT

SEQ ID NO: 288 - protein, 10 aa, synthetic construct
GGSIKSGSYY

SEQ ID NO: 289 - protein, 7 aa, synthetic construct
IYYSGST

SEQ ID NO: 290 - protein, 12 aa, synthetic construct
AREGSYPNQFDP

SEQ ID NO: 291 - protein, 6 aa, synthetic construct
QSVSSN

SEQ ID NO: 292 - skipped

SEQ ID NO: 293 - protein, 9 aa, synthetic construct
QQYHSFPFT

SEQ ID NO: 294 - protein, 120 aa, synthetic construct
QLQLQESGPGLVKPSETLSLTCTVSGGSIKSGSYYWGWIRQPPGKGLEWIGNIYYSGSTYYNPSLRSRVTISVDTSKNQFSLKLSSVTAADTAVYYCAREGSYPNQFDPWGQGTLVTVSS

SEQ ID NO: 295 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYHSFPFTFGGGTKVEIK

SEQ ID NO: 296 - protein, 450 aa, synthetic construct
QLQLQESGPGLVKPSETLSLTCTVSGGSIKSGSYYWGWIRQPPGKGLEWIGNIYYSGSTYYNPSLRSRVTISVDTSKNQFSLKLSSVTAADTAVYYCAREGSYPNQFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 297 - protein, 214 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYHSFPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 298 - protein, 5 aa, synthetic construct
DYNVN

SEQ ID NO: 299 - protein, 17 aa, synthetic construct
VINPKYGTTRYNQKFKG

SEQ ID NO: 300 - protein, 7 aa, synthetic construct
GLNAWDY

SEQ ID NO: 301 - protein, 11 aa, synthetic construct
GASENIYGALN

SEQ ID NO: 302 - protein, 7 aa, synthetic construct
GATNLED

SEQ ID NO: 303 - protein, 9 aa, synthetic construct
QNVLTTPYT

SEQ ID NO: 304 - protein, 8 aa, synthetic construct
GYSFTDYN

SEQ ID NO: 305 - protein, 8 aa, synthetic construct
INPKYGTT

SEQ ID NO: 306 - protein, 9 aa, synthetic construct
TRGLNAWDY

SEQ ID NO: 307 - protein, 6 aa, synthetic construct
ENIYGA

SEQ ID NO: 308 - skipped

SEQ ID NO: 309 - protein, 9 aa, synthetic construct
QNVLTTPYT

SEQ ID NO: 310 - protein, 116 aa, synthetic construct
QFQLVQSGAEVKKPGASVKVSCKASGYSFTDYNVNWVRQAPGQGLEWIGVINPKYGTTRYNQKFKGRATLTVDKSTSTAYMELSSLRSEDTAVYYCTRGLNAWDYWGQGTLVTVSS

SEQ ID NO: 311 - protein, 107 aa, synthetic construct
DIQMTQSPSSLSASVGDRVTITCGASENIYGALNWYQQKPGKAPKLLIYGATNLEDGVPSRFSGSGSGRDYTFTISSLQPEDIATYYCQNVLTTPYTFGQGTKLEIK

SEQ ID NO: 312 - protein, 446 aa, synthetic construct
QFQLVQSGAEVKKPGASVKVSCKASGYSFTDYNVNWVRQAPGQGLEWIGVINPKYGTTRYNQKFKGRATLTVDKSTSTAYMELSSLRSEDTAVYYCTRGLNAWDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 313 - protein, 214 aa, synthetic construct
DIQMTQSPSSLSASVGDRVTITCGASENIYGALNWYQQKPGKAPKLLIYGATNLEDGVPSRFSGSGSGRDYTFTISSLQPEDIATYYCQNVLTTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 314 - protein, 5 aa, synthetic construct
DYYIH

SEQ ID NO: 315 - protein, 17 aa, synthetic construct
RVNPDHGDSYYNQKFKD

SEQ ID NO: 316 - protein, 6 aa, synthetic construct
NYLFDH

SEQ ID NO: 317 - protein, 11 aa, synthetic construct
KASQDVGTAVA

SEQ ID NO: 318 - protein, 7 aa, synthetic construct
WASIRHT

SEQ ID NO: 319 - protein, 7 aa, synthetic construct
HQFATYT

SEQ ID NO: 320 - protein, 8 aa, synthetic construct
GYTFTDYY

SEQ ID NO: 321 - protein, 8 aa, synthetic construct
VNPDHGDS

SEQ ID NO: 322 - protein, 8 aa, synthetic construct
ARNYLFDH

SEQ ID NO: 323 - protein, 6 aa, synthetic construct
QDVGTA

SEQ ID NO: 324 - skipped

SEQ ID NO: 325 - protein, 7 aa, synthetic construct
HQFATYT

SEQ ID NO: 326 - protein, 115 aa, synthetic construct
EVQLVQSGAEVKKPGATVKISCKVSGYTFTDYYIHWVQQAPGKGLEWMGRVNPDHGDSYYNQKFKDKATITADKSTDTAYMELSSLRSEDTAVYFCARNYLFDHWGQGTLVTVSS

SEQ ID NO: 327 - protein, 105 aa, synthetic construct
DIQMTQSPSSVSASVGDRVTITCKASQDVGTAVAWYQQKPGKAPKLLIYWASIRHTGVPSRFSGSGSGTDFTLTISSLQPEDFATYYCHQFATYTFGGGTKVEIK

SEQ ID NO: 328 - protein, 445 aa, synthetic construct
EVQLVQSGAEVKKPGATVKISCKVSGYTFTDYYIHWVQQAPGKGLEWMGRVNPDHGDSYYNQKFKDKATITADKSTDTAYMELSSLRSEDTAVYFCARNYLFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 329 - protein, 212 aa, synthetic construct
DIQMTQSPSSVSASVGDRVTITCKASQDVGTAVAWYQQKPGKAPKLLIYWASIRHTGVPSRFSGSGSGTDFTLTISSLQPEDFATYYCHQFATYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 330 - protein, 10 aa, synthetic construct
GFTFSSYMMS

SEQ ID NO: 331 - protein, 17 aa, synthetic construct
TISGGGSNKYYVDSVKG

SEQ ID NO: 332 - protein, 7 aa, synthetic construct
QLYYFDY

SEQ ID NO: 333 - protein, 11 aa, synthetic construct
RASQESGIWLS

SEQ ID NO: 334 - protein, 7 aa, synthetic construct
TATSLAD

SEQ ID NO: 335 - protein, 9 aa, synthetic construct
QQVSVTPFT

SEQ ID NO: 336 - protein, 116 aa, synthetic construct
EVQLVESGGGLVQPGGSLRLSCAASGFTFSSYMMSWVRQAPGKGLEWVATISGGGSNKYYVDSVKGRFTISRDNAKNSLYLQMNSLRAEDTAVYYCARQLYYFDYWGQGTTVTVSS

SEQ ID NO: 337 - protein, 107 aa, synthetic construct
DIQMTQSPSSLSASVGDRVTITCRASQESGIWLSWYQQKPGKAPKLLIYTATSLADGVPSRFSGSGSGTDFTLTISSLQPEDFATYYCQQVSVTPFTFGGGTKVEIK

SEQ ID NO: 338 - protein, 7 aa, synthetic construct
AASSLQS

SEQ ID NO: 339 - protein, 107 aa, synthetic construct
DIQMTQSPSSLSASVGDRVTITCRASQESGIWLSWYQQKPGKAPKLLIYAASSLQSGVPSRFSGSGSGTDFTLTISSLQPEDFATYYCQQVSVTPFTFGGGTKVEIK

SEQ ID NO: 340 - protein, 7 aa, synthetic construct
TASSLAD

SEQ ID NO: 341 - protein, 107 aa, synthetic construct
DIQMTQSPSSLSASVGDRVTITCRASQESGIWLSWYQQKPGKAPKLLIYTASSLADGVPSRFSGSGSGTDFTLTISSLQPEDFATYYCQQVSVTPFTFGGGTKVEIK

SEQ ID NO: 342 - protein, 7 aa, synthetic construct
AASSLAD

SEQ ID NO: 343 - protein, 107 aa, synthetic construct
DIQMTQSPSSLSASVGDRVTITCRASQESGIWLSWYQQKPGKAPKLLIYAASSLADGVPSRFSGSGSGTDFTLTISSLQPEDFATYYCQQVSVTPFTFGGGTKVEIK

SEQ ID NO: 344 - protein, 1356 aa, synthetic construct
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 345 - protein, 1356 aa, synthetic construct
MASKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGVELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPVKYLGYPPPEIKWYKNGIPLESNHTVKVGHVLTIMEVSERDTGNYTVILTNPISKEKQSHVVSLVVYVPPQIGEKSLISPVDSYQYGTTQTLTCTVYAIPPPHHIHWYWQLEEECPNEPSQAVSVTNPYPCEEWRSVEDFQGGNKIEVNKNQFALIEGKNKTVSTLVIQAANVSALYKCEAVNKVGRGERVISFHVTRGPEITLQPDLQPTEQESVSLWCTADKSTFENLTWYKLGPQPLPVHVGELPTPVCKNLDTLWKLNATIFSNSTNDILIMELKNASLQDQGDYVCVAQDRKTKKRHCVVRQLTVLERVAPMITGNLENQTTSIGETIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLAPSFSGMVSSKSRESVASEGSNQTSGYQSGYHSDDTDTTVYSSEEAELLKLIEIGVQTGSTAQILQPDSGTTLSSPPV

SEQ ID NO: 346 - protein, 4 aa, synthetic construct
GGFG

(19) United States
(12) Patent Application Publication
(10) Pub. No.: US 2026/0265378 A1
(43) Pub. Date: Sep. 10, 2026

(54) COMBINATION THERAPY FOR CANCER TREATMENT

(71) Applicant: BioNTech SE, Mainz (DE)

(72) Inventor: Ugur SAHIN, Mainz (DE)

(21) Appl. No.: 19/638,378

(22) Filed: Apr. 3, 2026

Related U.S. Application Data
(63) Continuation of application No. PCT/EP2025/066589, filed on Jun. 13, 2025.

(30) Foreign Application Priority Data
Jun. 14, 2024 (WO) PCT/EP2024/066697
Jun. 14, 2024 (WO) PCT/EP2024/066699
Jun. 20, 2024 (WO) PCT/EP2024/067354
Sep. 30, 2024 (WO) PCT/EP2024/077507
Dec. 4, 2024 (WO) PCT/EP2024/084741
Mar. 19, 2025 (WO) PCT/EP2025/057557

(51) Int. Cl. C07K 16/28; A61K 39/00; A61K 47/68; A61P 35/00; C07K 16/22; C07K 16/30; C07K 16/32

(52) U.S. Cl. CPC C07K 16/2818; A61K 47/68031; A61K 47/68037; A61K 47/6849; A61K 47/6855; A61P 35/00; C07K 16/22; C07K 16/2827; C07K 16/2863; C07K 16/30; C07K 16/32; A61K 2039/505; C07K 2317/31; C07K 2317/569; C07K 2317/622; C07K 2317/76

(57) ABSTRACT

The invention relates to a combination of a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction, and an antibody-drug conjugate comprising an antibody or fragment thereof that specifically binds to a target expressed on the surface of a tumour cell.

### CROSS-REFERENCE TO RELATED APPLICATIONS

This application is a continuation of International Application Number PCT/EP2025/066589, which was filed on Jun. 13, 2025 and claims priority to International Application Numbers PCT/EP2025/057557, which was filed on Mar. 19, 2025; PCT/EP2024/084741, which was filed on Dec. 4, 2024; PCT/EP2024/077507, which was filed on Sep. 30, 2024; PCT/EP2024/067354, which was filed on Jun. 20, 2024; PCT/EP2024/066699 and PCT/EP2024/066697, which were filed on Jun. 14, 2024, the entirety of each of which is incorporated herein by reference.

### SEQUENCE LISTING

The computer-readable Sequence Listing submitted on Apr. 3, 2026 and identified as follows: 319,488 bytes ST.26 XML document file named “027820-8011-Sequence-Listing.xml,” created Mar. 31, 2026, is incorporated herein by reference in its entirety.

### FIELD OF THE INVENTION

The present application relates to the field of biomedicine, and in particular to a binding agent and an antibody-drug conjugate and uses thereof. The invention relates to a combination of a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction, and an antibody-drug conjugate comprising an antibody or fragment thereof that specifically binds to a target expressed on the surface of a tumour cell. Methods for using the binding agent and the antibody-drug conjugate of the present invention are also provided.

### BACKGROUND OF THE INVENTION

Antibody-drug conjugates (ADCs) and immunotherapy have each prompted a revolution in the treatment of cancer.

ADCs, comprising monoclonal antibodies conjugated to cytotoxic agents (payloads) via a linker, have emerged as novel therapeutic options. Upon binding with the cell surface antigen targeted by the specific antibody, the ADC is internalized by the tumour cell and processed by the endo-lysosomal system. Within the tumour cognate antigen-positive cells are bound by the antibody, the payload is released through the cleavable linker technology and results in cytotoxic activity.

ADCs can induce tumour cell death through the so-called bystander effect, which occurs when the cytotoxic warhead diffuses across the cell membrane to neighbouring cells, thus inducing their apoptosis. ADCs interact with cancer and immune cells by eliciting mechanisms such as immunogenic cell death, antibody-dependent cell-mediated cytotoxicity and dendritic cell activation, ultimately providing potential synergism with immunotherapy.

ADCs induce tumour-specific adaptive immunity, increasing the infiltration of T cells into the tumour microenvironment, whereas immune-checkpoint inhibitors reinvigorate exhausted T cells, enhancing antitumour immune responses.

The approval of immunotherapy, such as immune checkpoint inhibitors (ICIs) targeting programmed cell death protein 1 (PD-1) and programmed death-ligand 1 (PD-L1) has dramatically changed the treatment landscape for cancer subjects, and, combination therapy targeting multiple immune checkpoints has recently been explored to provide greater clinical benefits and broader indications.

Despite these promising improvements, response to immunotherapy treatment is not guaranteed in all cancer subjects and only a limited fraction of patients obtained a long-term benefit of ADCs and immunotherapy.

There is therefore an unmet need for improved methods for cancer treatment. The effect of combining any given ADC and any given binding agent in a combination therapy cannot always be predicted.

### SUMMARY OF THE INVENTION

Against the aforementioned background, it is an object of the present invention to provide effective pharmacological means to treat cancer. It is also an object of the present invention to provide pharmacological means to elicit a therapeutically effective immune response for the treatment of cancer.

These objects are achieved by the invention set forth in the claims and embodiments explained in more detail below.

In some aspects of the invention, there is provided a combination comprising: a. a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction; and b. an antibody-drug conjugate comprising an antibody or fragment thereof that specifically binds to a target expressed on the surface of a tumour cell.

In some aspects of the invention, there is provided a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction; for use in treating cancer in a subject, the use comprising administering to the subject: a. the binding agent; and b. an antibody-drug conjugate comprising an antibody or fragment thereof that specifically binds to a target expressed on the surface of a tumour cell.

In some aspects of the invention, there is provided an antibody-drug conjugate comprising an antibody or fragment thereof that specifically binds to a target expressed on the surface of a tumour cell for use in treating cancer in a subject, the use comprising administering to the subject: a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction; and b. the antibody-drug conjugate.

In some aspects of the invention, there is provided an antibody-drug conjugate comprising an antibody or fragment thereof that specifically binds to a target expressed on the surface of a tumour cell and a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction; for use in therapy, preferably for treating cancer in a subject, preferably the use comprising administering to the subject: a. the binding agent; and b. the antibody-drug conjugate.

In some aspects of the invention, a method of treating cancer in a subject is provided, the method comprising administering to the subject: a. a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction; and b. an antibody-drug conjugate comprising an antibody or fragment thereof that specifically binds to a target expressed on the surface of a tumour cell.

In some aspects of the invention, a composition is provided comprising: a. a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction; and b. an antibody-drug conjugate comprising an antibody or fragment thereof that specifically binds to a target expressed on the surface of a tumour cell.

In some aspects of the invention, a kit-of-parts is provided comprising: a. a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction; and b. an antibody-drug conjugate comprising an antibody or fragment thereof that specifically binds to a target expressed on the surface of a tumour cell.

In some aspects of the invention, there is provided a kit comprising:
(a) a composition comprising a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction; and
(b) a composition comprising an antibody-drug conjugate comprising an antibody or fragment thereof that specifically binds to a target expressed on the surface of a tumour cell.

In some aspects of the invention, there is provided use of a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction; in the manufacture of a medicament for the treatment of cancer in a subject, the use comprising administering to the subject: a. the binding agent; and b. an antibody-drug conjugate comprising an antibody or fragment thereof that specifically binds to a target expressed on the surface of a tumour cell.

In some aspects of the invention, there is provided use of an antibody-drug conjugate comprising an antibody or fragment thereof that specifically binds to a target expressed on the surface of a tumour cell in the manufacture of a medicament for the treatment of cancer in a subject, the use comprising administering to the subject: a. a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction; and b. the antibody-drug conjugate.

In some aspects of the invention, there is provided use of an antibody-drug conjugate comprising an antibody or fragment thereof that specifically binds to a target expressed on the surface of a tumour cell and a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction; in the manufacture of a medicament for therapy, preferably for treating cancer in a subject, preferably the use comprising administering to the subject: a. the binding agent; and b. the antibody-drug conjugate.

In some aspects of the invention, there is provided use of a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction; in treating cancer in a subject, the use comprising administering to the subject: a. the binding agent; and b. an antibody-drug conjugate comprising an antibody or fragment thereof that specifically binds to a target expressed on the surface of a tumour cell.

In some aspects of the invention, there is provided use of an antibody-drug conjugate comprising an antibody or fragment thereof that specifically binds to a target expressed on the surface of a tumour cell in treating cancer in a subject, the use comprising administering to the subject: a. a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction; and b. the antibody-drug conjugate.

In some aspects of the invention, there is provided use of an antibody-drug conjugate comprising an antibody or fragment thereof that specifically binds to a target expressed on the surface of a tumour cell and a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction; for therapy, preferably for treating cancer in a subject, preferably the use comprising administering to the subject: a. the binding agent; and b. the antibody-drug conjugate.

In some aspects of the invention, there is provided a combination of:
a. a bispecific antibody that comprises:
   (a) an antibody or fragment thereof that specifically binds to PD-L1, and
   (b) an antibody or fragment thereof that specifically binds to VEGF; and
b. an antibody-drug conjugate comprising an antibody or fragment thereof that specifically binds to a target expressed on the surface of a tumour cell.

In some aspects of the invention, there is provided a bispecific antibody that comprises:
(a) an antibody or fragment thereof that specifically binds to PD-L1, and
(b) an antibody or fragment thereof that specifically binds to VEGF;
   for use in treating cancer in a subject, the use comprising administering to the subject:
a. the bispecific antibody; and
b. an antibody-drug conjugate comprising an antibody or fragment thereof that specifically binds to a target expressed on the surface of a tumour cell.

In some aspects of the invention, there is provided an antibody-drug conjugate comprising an antibody or fragment thereof that specifically binds to a target expressed on the surface of a tumour cell for use in treating cancer in a subject, the use comprising administering to the subject:
a. a bispecific antibody that comprises:
   (a) an antibody or fragment thereof that specifically binds to PD-L1, and
   (b) an antibody or fragment thereof that specifically binds to VEGF; and
b. the antibody-drug conjugate.

In some aspects of the invention, there is provided an antibody-drug conjugate comprising an antibody or fragment thereof that specifically binds to a target expressed on the surface of a tumour cell and a bispecific antibody that comprises:
(a) an antibody or fragment thereof that specifically binds to PD-L1, and
(b) an antibody or fragment thereof that specifically binds to VEGF;
   for use in therapy, preferably for treating cancer in a subject, preferably the use comprising administering to the subject:
a. the bispecific antibody; and
b. the antibody-drug conjugate.

In some aspects of the invention, a method of treating cancer in a subject is provided, the method comprising administering to the subject:
a. a bispecific antibody that comprises:
   (a) an antibody or fragment thereof that specifically binds to PD-L1, and
   (b) an antibody or fragment thereof that specifically binds to VEGF; and
b. an antibody-drug conjugate comprising an antibody or fragment thereof that specifically binds to a target expressed on the surface of a tumour cell.

In some aspects of the invention, a composition is provided comprising:
a. a bispecific antibody that comprises:
   (a) an antibody or fragment thereof that specifically binds to PD-L1, and
   (b) an antibody or fragment thereof that specifically binds to VEGF; and
b. an antibody-drug conjugate comprising an antibody or fragment thereof that specifically binds to a target expressed on the surface of a tumour cell.

In some aspects of the invention, a kit-of-part is provided comprising:
a. a bispecific antibody that comprises:
   (a) an antibody or fragment thereof that specifically binds to PD-L1, and
   (b) an antibody or fragment thereof that specifically binds to VEGF; and
b. an antibody-drug conjugate comprising an antibody or fragment thereof that specifically binds to a target expressed on the surface of a tumour cell.

In some aspects of the invention, there is provided use of a bispecific antibody that comprises:
(a) an antibody or fragment thereof that specifically binds to PD-L1, and
(b) an antibody or fragment thereof that specifically binds to VEGF;
   in the manufacture of a medicament for the treatment of cancer in a subject, the use comprising administering to the subject:
a. the bispecific antibody; and
b. an antibody-drug conjugate comprising an antibody or fragment thereof that specifically binds to a target expressed on the surface of a tumour cell.

In some aspects of the invention, there is provided use of an antibody-drug conjugate comprising an antibody or fragment thereof that specifically binds to a target expressed on the surface of a tumour cell in the manufacture of a medicament for the treatment of cancer in a subject, the use comprising administering to the subject:
a. a bispecific antibody that comprises:
   (a) an antibody or fragment thereof that specifically binds to PD-L1, and
   (b) an antibody or fragment thereof that specifically binds to VEGF; and
b. the antibody-drug conjugate.

In some aspects of the invention, there is provided use of an antibody-drug conjugate comprising an antibody or fragment thereof that specifically binds to a target expressed on the surface of a tumour cell and a bispecific antibody that comprises:
(a) an antibody or fragment thereof that specifically binds to PD-L1, and
(b) an antibody or fragment thereof that specifically binds to VEGF;
   in the manufacture of a medicament for therapy, preferably for treating cancer in a subject, preferably the use comprising administering to the subject:
a. the bispecific antibody; and
b. the antibody-drug conjugate.

In some aspects of the invention, there is provided use of a bispecific antibody that comprises:
(a) an antibody or fragment thereof that specifically binds to PD-L1, and
(b) an antibody or fragment thereof that specifically binds to VEGF;
   in treating cancer in a subject, the use comprising administering to the subject:
a. the bispecific antibody; and
b. an antibody-drug conjugate comprising an antibody or fragment thereof that specifically binds to a target expressed on the surface of a tumour cell.

In some aspects of the invention, there is provided use of an antibody-drug conjugate comprising an antibody or fragment thereof that specifically binds to a target expressed on the surface of a tumour cell in treating cancer in a subject, the use comprising administering to the subject:
a. a bispecific antibody that comprises:
   (a) an antibody or fragment thereof that specifically binds to PD-L1, and
   (b) an antibody or fragment thereof that specifically binds to VEGF; and
b. the antibody-drug conjugate.

In some aspects of the invention, there is provided use of an antibody-drug conjugate comprising an antibody or fragment thereof that specifically binds to a target expressed on the surface of a tumour cell and a bispecific antibody that comprises:
(a) an antibody or fragment thereof that specifically binds to PD-L1, and
(b) an antibody or fragment thereof that specifically binds to VEGF;
   for therapy, preferably for treating cancer in a subject, preferably the use comprising administering to the subject:
a. the bispecific antibody; and
b. the antibody-drug conjugate.

### BRIEF DESCRIPTION OF THE FIGURES

FIG. 1: Anti-tumour efficacy study in a syngeneic hHER2-MC38 tumour model. The combination of anti-hHER2-ADC with anti-mhPDL1xmVEGF resulted in tumour regression while in corresponding monotherapy groups tumour growth was only reduced.

FIG. 2: Anti-tumour efficacy study in a syngeneic hHER2-MC38 tumour model. The combination of anti-hHER2-ADC with anti-mhPDL1xmVEGF resulted in significantly lower tumour volumes at the termination of the study (day 24 after treatment start) compared to the corresponding monotherapies. G2 vs. G5, G2 vs. G6, G3 vs. G5 and G4 vs. G6 were compared with t test, **p<0.01, ***p<0.001.

FIG. 3: Anti-tumour efficacy study in an A375 xenograft PBMC-co-injected tumour model. The combination of anti-hHER2-ADC with anti-hPDL1xhVEGF showed superior tumour growth inhibition compared to the corresponding monotherapies.

FIG. 4: Anti-tumour efficacy study in an A375 xenograft PBMC-co-injected tumour model. The combination of anti-hHER2-ADC with anti-hPDL1xhVEGF resulted in lower tumour weights at the termination of the study (day 29 post inoculation) compared to the corresponding monotherapies.

FIG. 5: Anti-tumour efficacy study in a syngeneic hB7-H3-CT26 tumour model. The combination of anti-hB7-H3-ADC with anti-mPDL1xmVEGF resulted in tumour regression and long-term tumour control while in corresponding monotherapy groups tumour growth was only reduced.

FIG. 6: Anti-tumour efficacy study in a syngeneic hB7-H3-CT26 tumour model. The combination of anti-hHER2-ADC with anti-mPDL1xmVEGF resulted in significantly lower tumour weights at the termination of the study (day 28 post inoculation) compared to the corresponding monotherapies. G2 vs. G4, G3 vs. G4 were compared with t test, *p<0.05.

FIG. 7: Anti-tumour efficacy study in an A375 xenograft PBMC-co-injected tumour model. The combination of anti-hB7-H3-ADC with anti-hPDL1xhVEGF showed superior tumour growth inhibition compared to the corresponding monotherapies.

FIG. 8: Anti-tumour efficacy study in an A375 xenograft PBMC-co-injected tumour model. The combination of anti-hB7-H3-ADC with anti-hPDL1xhVEGF resulted in significantly lower tumour weights at the termination of the study (day 24 post inoculation) compared to the corresponding monotherapies. G2 vs. G4, G3 vs. G4 were compared with t test, *p<0.05.

FIG. 9: Anti-tumour efficacy study in a syngeneic hTROP2-MC38 tumour model. The combination of anti-hTROP2-ADC with anti-mPDL1xmVEGF resulted in complete tumour growth regression and long-term tumour control while in corresponding monotherapy groups tumour growth was only reduced.

FIG. 10: Anti-tumour efficacy study in a syngeneic hTROP2-MC38 tumour model. The combination of anti-hTROP2-ADC with anti-mPDL1xmVEGF resulted in lower tumour weights at the termination of the study (day 28 after treatment start) compared to the corresponding monotherapies. Statistical analysis via One-way ANOVA followed Dunnett's test on mean tumour weight of the treatment group versus 0.9% sodium chloride injection group on study endpoint, *p<0.05, ***p<0.001, ****p<0.0001.

FIG. 11: Anti-tumour efficacy study in an A375 xenograft PBMC-co-injected tumour model. The combination of anti-hTROP2-ADC with anti-hPDL1xhVEGF resulted in tumour regression and long-term tumour control while in corresponding monotherapy groups tumour growth was only reduced.

FIG. 12: Anti-tumour efficacy study in an A375 xenograft PBMC-co-injected tumour model. The combination of anti-hTROP2-ADC with anti-hPDL1xhVEGF resulted in lower tumour weights at the termination of the study (day 29 post inoculation) compared to the corresponding monotherapies.

FIG. 13 shows tumour growth inhibition by an anti-HER3-ADC (Compound 4)+anti-mPDL1xmVEGF surrogate (mouse surrogate of Compound 5, in which the anti-VEGF part is changed to an anti-mouse-VEGF part and the anti-PDL1 VHH domain is exchanged to a scFv of atezolizumab, which is mouse cross-reactive) in a syngeneic MC38-hHER3 model.

FIG. 14 shows tumour growth inhibition by an anti-HER3-ADC (Compound 4)+anti-hPDL1xhVEGF (Compound 5) in an A375 xenograft hPBMC model.

FIG. 15 shows tumour growth inhibition by an anti-HER3-ADC (Compound 4)+ and anti-mPDL1xmVEGF (mouse surrogate of Compound 5) compared with each compound as monotherapy, in a syngeneic mouse hHER3-MC38 model.

FIG. 16 shows body weight measurements by an anti-HER3-ADC (Compound 4)+ and anti-mPDL1xmVEGF (mouse surrogate of Compound 5) combination compared with each compound as monotherapy.

FIG. 17 shows survival analysis for the anti-HER3-ADC (Compound 4)+anti-mPDL1xmVEGF (mouse surrogate of Compound 5) combination compared with the respective monotherapy groups.

FIG. 18 shows tumour growth inhibition by an anti-HER2-ADC (Compound 1)+anti-hPD1xhVEGF (Compound 13) in a RKO xenograft hPBMC model.

FIG. 19 shows tumour growth inhibition by an anti-B7-H3-ADC (Compound 2)+anti-hPD1xhVEGF (Compound 13) in a RKO xenograft hPBMC model.

### DETAILED DESCRIPTION

Although certain embodiments of the present invention are described in detail below, it is to be understood that this invention is not limited to the particular embodiments, methodologies, protocols and reagents described herein as these may vary within the scope set by the claims.

It is also to be understood that terminology used herein is for the purpose of describing particular embodiments only, and is not intended to limit the scope of the present invention which is defined by the appended claims. Unless defined otherwise, all technical and scientific terms used herein have the same meanings as commonly understood by one of ordinary skill in the art.

In the following description, certain elements of the present invention will be described. These elements may be discussed with specific embodiments, however, it should be understood that they may be combined in any manner and in any number to create additional embodiments. The variously described examples, features and particular embodiments should not be construed to limit the present invention to only the explicitly described embodiments or to the explicitly described combination of features. This description should be understood to disclose and encompass embodiments which combine the explicitly described embodiments with any number of the disclosed and/or preferred elements. Furthermore, any permutations and combinations of all described elements in this application should be considered disclosed by this description unless the context indicates otherwise. For example, if in a preferred embodiment of the binding agent described herein, the binding agent comprises one or two anti-PD-L1 single domain antibodies (e.g., one or two anti-PD-L1 VHHs) and, in another preferred embodiment of the binding agent described herein, the binding agent comprises a second binding region which binds to VEGF-A, then in a further preferred embodiment of the binding agent described herein, the binding agent comprises one or two anti-PD-L1 single domain antibodies (e.g., one or two anti-PD-L1 VHHs) and a second binding region which binds to VEGF-A.

### Embodiments

The above objects are achieved by the following embodiments in accordance with the invention:

1. A combination of
a. a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction, and
b. an antibody-drug conjugate comprising an antibody or fragment thereof that specifically binds to a target expressed on the surface of a tumour cell.

2. A binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction, for use in treating cancer in a subject, the use comprising administering to the subject:
a. the binding agent; and
b. an antibody-drug conjugate comprising an antibody or fragment thereof that specifically binds to a target expressed on the surface of a tumour cell.

3. An antibody-drug conjugate comprising an antibody or fragment thereof that specifically binds to a target expressed on the surface of a tumour cell for use in treating cancer in a subject, the use comprising administering to the subject:
a. a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction; and
b. the antibody-drug conjugate.

4. An antibody-drug conjugate comprising an antibody or fragment thereof that specifically binds to a target expressed on the surface of a tumour cell and a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction, for use in therapy, preferably for treating cancer in a subject, preferably the use comprising administering to the subject:
a. the binding agent; and
b. the antibody-drug conjugate.

5. The combination according to embodiment 1, binding agent for use according to embodiment 2, antibody-drug conjugate for use according to embodiment 3, or antibody-drug conjugate and binding agent for use according to embodiment 4, wherein the target expressed on the surface of a tumour cell is HER2, B7H3, TROP2, HER3, CD30, Nectin-4, TF, B7-H4, and/or ITGB6, preferably HER2, B7H3, TROP2 and/or HER3.

6. The combination according to embodiment 1 or 5, binding agent for use according to embodiment 2 or 5, antibody-drug conjugate for use according to embodiment 3 or 5, or antibody-drug conjugate and binding agent for use according to embodiment 4 or 5, wherein the antibody or fragment thereof that specifically binds to a target expressed on the surface of a tumour cell is selected from the group consisting of a human antibody, a humanized antibody, a chimeric antibody, a multispecific antibody, a monoclonal antibody, and a polyclonal antibody.

7. The combination according to embodiment 1 or 5, binding agent for use according to embodiment 2 or 5, antibody-drug conjugate for use according to embodiment 3 or 5, or antibody-drug conjugate and binding agent for use according to embodiment 4 or 5, wherein the antibody or fragment thereof that specifically binds to a target expressed on the surface of a tumour cell is selected from the group consisting of an Fab, an Fab′, an F(ab′)2, an Fv, an ScFv, an Fab′-SH, an sdAb, and a VHH.

8. The combination according to any of embodiments 1 or 5-7, binding agent for use according to any of embodiments 2 or 5-7, antibody-drug conjugate for use according to any of embodiments 3 or 5-7, or antibody-drug conjugate and binding agent for use according to any of embodiments 4-7, wherein the antibody-drug conjugate comprises an immunoglobulin constant region, the immunoglobulin constant region being a human IgG constant region, e.g., a human IgG1 constant region.

9. The combination according to any of embodiments 1 or 5-8, binding agent for use according to any of embodiments 2 or 5-8, antibody-drug conjugate for use according to any of embodiments 3 or 5-8, or antibody-drug conjugate and binding agent for use according to any of embodiments 4-8, wherein the antibody-drug conjugate comprises said antibody or fragment thereof linked to a payload by a linker, preferably wherein the payload is a cytotoxic drug.

10. The combination according to embodiment 9, binding agent for use according to embodiment 9, antibody-drug conjugate for use according to embodiment 9, or antibody-drug conjugate and binding agent for use according to embodiment 9, wherein the linker is a cleavable linker, preferably wherein the linker is cleavable by a lysosomal protease.

11. The combination according to embodiment 9 or 10, binding agent for use according to embodiment 9 or 10, antibody-drug conjugate for use according to embodiment 9 or 10, or antibody-drug conjugate and binding agent for use according to embodiment 9 or 10, wherein the linker comprises or is a peptide linker, preferably wherein the peptide linker consists of 1 to 10 amino acid residues, more preferably wherein the peptide linker consists of 1 to 6 amino acid residues.

12. The combination according to embodiment 11, binding agent for use according to embodiment 11, antibody-drug conjugate for use according to embodiment 11, or antibody-drug conjugate and binding agent for use according to embodiment 11, wherein the peptide linker consists of 3 amino acid residues.

13. The combination according to embodiment 11, binding agent for use according to embodiment 11, antibody-drug conjugate for use according to embodiment 11, or antibody-drug conjugate and binding agent for use according to embodiment 11, wherein the peptide linker consists of 4 amino acid residues.

14. The combination according to embodiment 11, binding agent for use according to embodiment 11, antibody-drug conjugate for use according to embodiment 11, or antibody-drug conjugate and binding agent for use according to embodiment 11, wherein the peptide linker comprises any of AA¹, AA¹-Gly, Val-Cit, Val-Ala, Val-AA¹, Val-AA¹-Gly, AA¹-Ala-Asn, Ala-Ala-Ala, Ala-Ala-Asn or Gly-Gly-Phe-Gly (GGFG).

15. The combination according to embodiment 14, binding agent for use according to embodiment 14, antibody-drug conjugate for use according to embodiment 14, or antibody-drug conjugate and binding agent for use according to embodiment 14, wherein the amino acid residue represented by AA is selected from

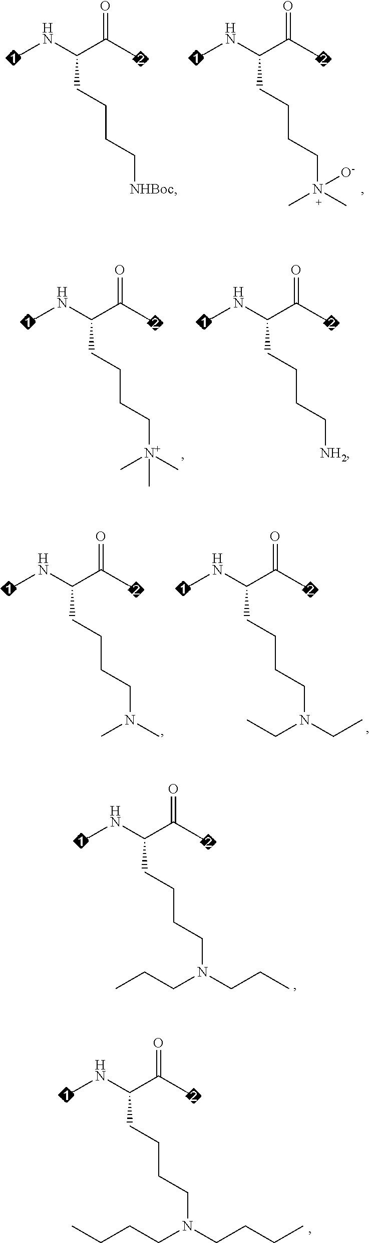

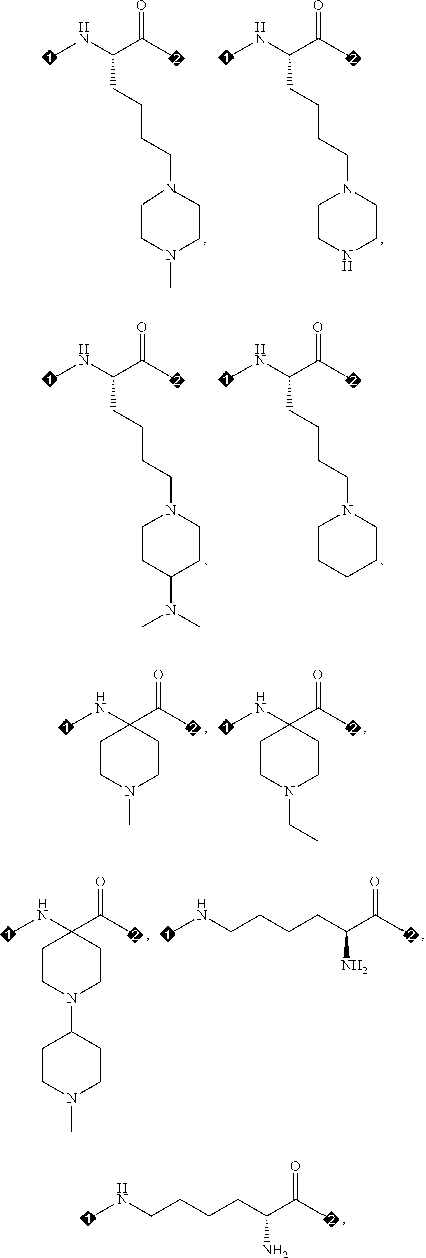

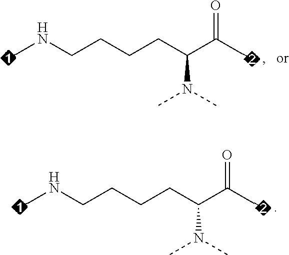

16. The combination according to embodiment 14, binding agent for use according to embodiment 14, antibody-drug conjugate for use according to embodiment 14, or antibody-drug conjugate and binding agent for use according to embodiment 14, wherein the peptide linker comprises a GGFG peptide moiety.

17. The combination according to any of embodiments 9-16, binding agent for use according to any of embodiments 9-16, antibody-drug conjugate for use according to any of embodiments 9-16, or antibody-drug conjugate and binding agent for use according to any of embodiments 9-16, wherein the linker comprises a first conjugation moiety for coupling with the antibody.

18. The combination according to embodiment 17, binding agent for use according to embodiment 17, antibody-drug conjugate for use according to embodiment 17, or antibody-drug conjugate and binding agent for use according to embodiment 17, wherein the first conjugation moiety comprises a pyrimidine-sulfone moiety, preferably wherein the pyrimidine-sulfone moiety is linked to the peptide moiety via a C1-10 alkynoyl group.

19. The combination according to embodiment 17, binding agent for use according to embodiment 17, antibody-drug conjugate for use according to embodiment 17, or antibody-drug conjugate and binding agent for use according to embodiment 17, wherein the first conjugation moiety comprises a maleimide moiety, preferably wherein the maleimide moiety is linked to the peptide moiety via a C1-10 alkanoyl group.

20. The combination according to any of embodiments 9-19, binding agent for use according to any of embodiments 9-19, antibody-drug conjugate for use according to any of embodiments 9-19, or antibody-drug conjugate and binding agent for use according to any of embodiments 9-19, wherein the linker comprises a second conjugation moiety for coupling with the payload.

21. The combination according to any of embodiments 9-20, binding agent for use according to any of embodiments 9-20, antibody-drug conjugate for use according to any of embodiments 9-20, or antibody-drug conjugate and binding agent for use according to any of embodiments 9-20, wherein the payload of the linker-payload moiety is a tubulin polymerisation inhibitor, a DNA damaging agent, a TOPO1 inhibitor, an auristatin, a maytansinoid, or a calicheamicin.

22. The combination according to any one of embodiments 9-21, binding agent for use according to any one of embodiments 9-21, antibody-drug conjugate for use according to any one of embodiments 9-21, or antibody-drug conjugate and binding agent for use according to any one of embodiments 9-21, wherein the payload is a TOPO1 inhibitor.

23. The combination according to embodiment 22, binding agent for use according to embodiment 22, antibody-drug conjugate for use according to embodiment 22, or antibody-drug conjugate and binding agent for use according to embodiment 22, wherein the TOPO1 inhibitor is a camptothecin or an exatecan, or a derivative thereof.

24. The combination according to embodiment 22, binding agent for use according to embodiment 22, antibody-drug conjugate for use according to embodiment 22, or antibody-drug conjugate and binding agent for use according to embodiment 22, wherein the TOPO1 inhibitor is deruxtecan.

25. The combination according to any one of embodiments 9-24, binding agent for use according to any one of embodiments 9-24, antibody-drug conjugate for use according to any one of embodiments 9-24, or antibody-drug conjugate and binding agent for use according to any one of embodiments 9-24, wherein the payload of the linker-payload moiety of the antibody-drug conjugate comprises a structure shown as formula (V-A):

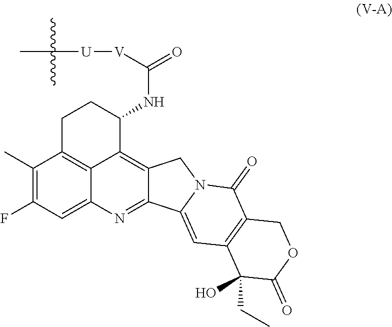

or a tautomer, mesomer, enantiomer, a diastereoisomer thereof, or a pharmaceutically acceptable salt thereof wherein,
U is O or S; and
V is (C1-6) alkylene, (C3-10) cycloalkylene, or (C1-6) alkylene-(C3-10) cycloalkylene.

26. The combination according to any one of embodiments 9-25, binding agent for use according to any one of embodiments 9-25, antibody-drug conjugate for use according to any one of embodiments 9-25, or antibody-drug conjugate and binding agent for use according to any one of embodiments 9-25, wherein the linker-payload moiety of the antibody-drug conjugate comprises a structure shown as formula (V-C):

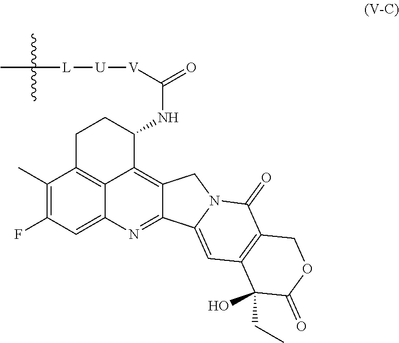

or a tautomer, mesomer, enantiomer a diastereoisomer thereof, or a pharmaceutically acceptable salt thereof wherein,
L is —La-Lb-Lc-;
-La- is selected from the group consisting of

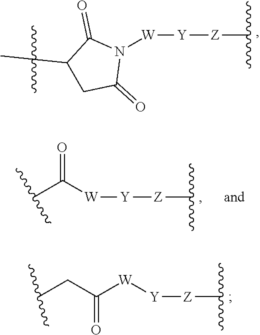

wherein W is —(C(Rwa)(Rwb))wn-, Y is —(OCH2CH2)yn-Oyp, and Z is —(C(Rza)(Rzb))zn; wherein wn is selected from the group consisting of integers ≥0, and
0 or at least 1 methylene unit of W is independently replaced by -Cyr-, —N(Rwx)C(O)—, —C(O)N(Rwx)-, —C(O)—, —OC(O)—, —C(O)O—, —NRwx-, —O—, —S—, —SO—, —SO2-, —P(Rwx)-, —P(═O)(Rwx)-, —N(Rwx)SO2-, —SO2N(Rwx)-, —C(═S)—, —C(═NRwx)-, —N═N—, —C═N—, —N═C— or —C(═N2)-;
wherein yn is selected from the group consisting of integers ≥0, and yp is 0 or 1;
wherein zn is selected from the group consisting of integers ≥0, and
0 or at least 1 methylene unit of Z is independently replaced by -Cyr-, —N(Rzx)C(O)—, —C(O)N(Rzx)-, —C(O)—, —OC(O)—, —C(O)O—, —NRzx-, —O—, —S—, —SO—, —SO2-, —P(Rzx)-, —P(═O)(Rzx)-, —N(Rzx)SO2-, —SO2N(Rzx)-, —C(═S)—, —C(═NRzx)-, —N═N—, —C═N—, —N═C— or —C(═N2)-;
Cyr- is selected from the group consisting of 6-10 membered arylene, 5-8 membered heteroarylene, 3-10 membered heterocyclylene and 3-10 membered saturated or partially unsaturated carbocyclylene, wherein -Cyr- is unsubstituted or independently substituted with at least 1 substituent Rcx;
wherein each Rwa, each Rwb, each Rza, each Rzb, each Rwx, each Rzx and each Rcx are each independently hydrogen, protium, deuterium, tritium, halogen, —NO2, —CN, —ORr, —SRr, —N(Rra)(Rrb), —C(O)Rr, —CO2Rr, —C(O)C(O)Rr, —C(O)CH2C(O)Rr, —S(O)Rr, —S(O)2Rr, —C(O)N(Rra)(Rrb), —SO2N(Rra)(Rrb), —OC(O)Rr, —N(R)SO2Rr, or a C1-6 aliphatic group optionally substituted with Rr;
wherein each Rr, each Rra and each Rrb are each independently hydrogen, protium, deuterium, tritium, halogen, —NO2, —CN, —OH, —SH, —NH2, —C(O)H, —CO2H, —C(O)C(O)H, —C(O)CH2C(O)H, —S(O)H, —S(O)2H, —C(O)NH2, —SO2NH2, —OC(O)H, —N(H)SO2H or a C1-6 aliphatic group;
Lb- represents a peptide residue consisting of 2 to 7 amino acids;
-Lc- is selected from the group consisting of

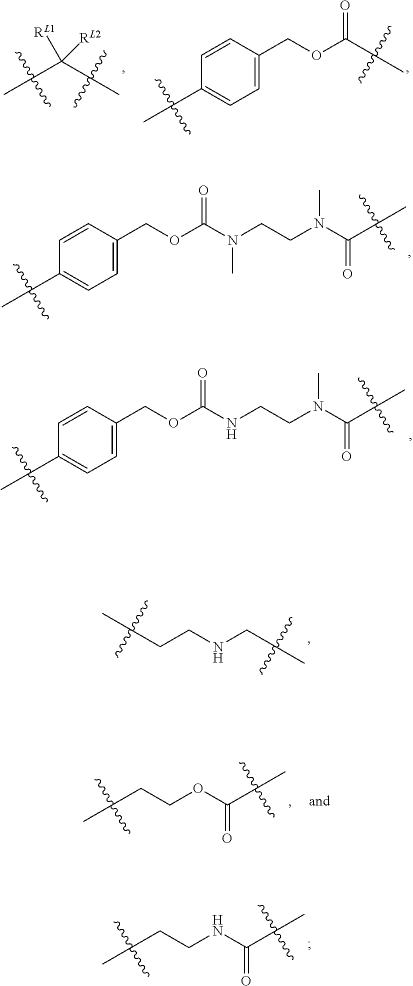

wherein RL1 and RL2 are each independently selected from the group consisting of hydrogen, protium, deuterium, tritium, halogen, —NO2, —CN, —OH, —SH, —NH2, —C(O)H, —CO2H, —C(O)C(O)H, —C(O)CH2C(O)H, —S(O)H, —S(O)2H, —C(O)NH2, —SO2NH2, —OC(O)H, —N(H)SO2H and a C1-6 aliphatic group;
U is O or S; and
V is (C1-6) alkylene, (C3-10) cycloalkylene, or (C1-6) alkylene-(C3-10) cycloalkylene.

27. The combination according to any one of embodiments 9-26, binding agent for use according to any one of embodiments 9-26, antibody-drug conjugate for use according to any one of embodiments 9-26, or antibody-drug conjugate and binding agent for use according to any one of embodiments 9-26, wherein the antibody-drug conjugate has a structure shown as formula (V-D):

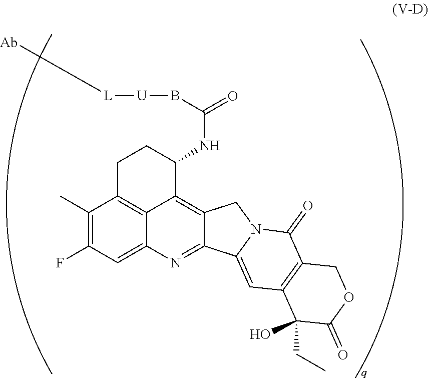

or a tautomer, mesomer, enantiomer or diastereoisomer thereof, or a pharmaceutically acceptable salt thereof wherein:
Ab is an antibody or fragment thereof that specifically binds to a target expressed on the surface of a tumour cell;
the connection number q is an integer from 1 to 16, and
L is -La-Lb-Lc-;
-La- is selected from the group consisting of

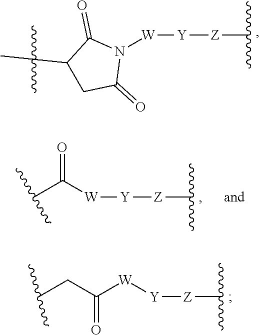

wherein W is —(C(Rwa)(Rwb))wn-, Y is —(OCH2CH2)yn-Oyp, and Z is —(C(Rza)(Rzb))zn; wherein wn is selected from the group consisting of integers ≥0, and 0 or at least 1 methylene unit of W is independently replaced by -Cyr-, —N(Rwx)C(O)—, —C(O)N(Rwx)-, —C(O)—, —OC(O)—, —C(O)O—, —NRwx-, —O—, —S—, —SO—, —SO2-, —P(Rwx)-, —P(═O)(Rwx)-, —N(Rwx)SO2-, —SO2N(Rwx)-, —C(═S)—, —C(═NRwx)-, —N═N—, —C═N—, —N═C— or —C(═N2)-;
wherein yn is selected from the group consisting of integers ≥0, and yp is 0 or 1; wherein zn is selected from the group consisting of integers ≥0, and
0 or at least 1 methylene unit of Z is independently replaced by -Cyr-, —N(Rzx)C(O)—, —C(O)N(Rzx)-, —C(O)—, —OC(O)—, —C(O)O—, —NRzx-, —O—, —S—, —SO—, —SO2-, —P(Rzx)-, —P(═O)(Rzx)-, —N(Rzx)SO2-, —SO2N(Rzx)-, —C(═S)—, —C(═NRzx)-, —N═N—, —C═N—, —N═C— or —C(═N2)-;
-Cyr- is selected from the group consisting of 6-10 membered arylene, 5-8 membered heteroarylene, 3-10 membered heterocyclylene and 3-10 membered saturated or partially unsaturated carbocyclylene, wherein -Cyr- is unsubstituted or independently substituted with at least 1 substituent Rcx;
wherein each Rwa, each Rwb, each Rza, each Rzb, each Rwx, each Rzx and each Rcx are each independently hydrogen, protium, deuterium, tritium, halogen, —NO2, —CN, —ORr, —SRr, —N(Rra)(Rrb), —C(O)Rr, —CO2Rr, —C(O)C(O)Rr, —C(O)CH2C(O)Rr, —S(O)Rr, —S(O)2Rr, —C(O)N(Rra)(Rrb), —SO2N(Rra)(Rrb), —OC(O)Rr, —N(R)SO2Rr, or a C1-6 aliphatic group optionally substituted with Rr;
wherein each Rr, each Rra and each Rrb are each independently hydrogen, protium, deuterium, tritium, halogen, —NO2, —CN, —OH, —SH, —NH2, —C(O)H, —CO2H, —C(O)C(O)H, —C(O)CH2C(O)H, —S(O)H, —S(O)2H, —C(O)NH2, —SO2NH2, —OC(O)H, —N(H)SO2H or a C1-6 aliphatic group;
-Lb- represents a peptide residue consisting of 2 to 7 amino acids;
-Lc- is selected from the group consisting of

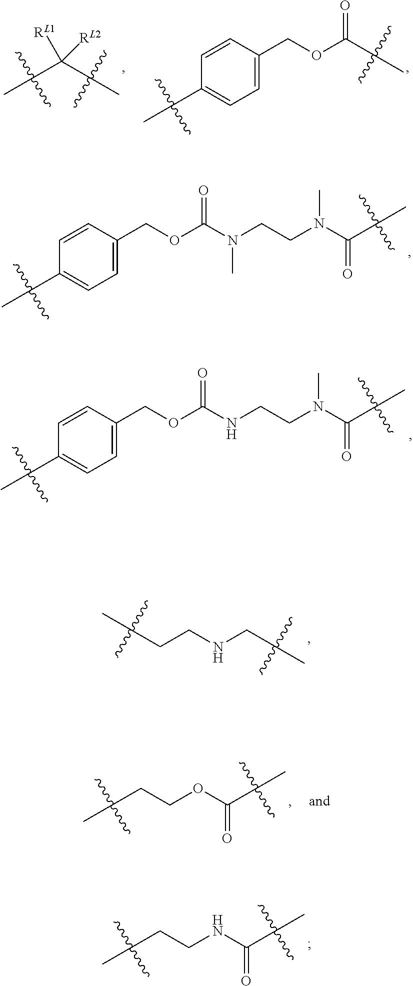

wherein RL1 and RL2 are each independently selected from the group consisting of hydrogen, protium, deuterium, tritium, halogen, —NO2, —CN, —OH, —SH, —NH2, —C(O)H, —CO2H, —C(O)C(O)H, —C(O)CH2C(O)H, —S(O)H, —S(O)2H, —C(O)NH2, —SO2NH2, —OC(O)H, —N(H)SO2H and a C1-6 aliphatic group;
U is O or S; and
V is (C1-6) alkylene, (C3-10) cycloalkylene, or (C1-6) alkylene-(C3-10) cycloalkylene.

28. The combination according to any of embodiments 9-27, binding agent for use according to any of embodiments 9-27, antibody-drug conjugate for use according to any of embodiments 9-27, or antibody-drug conjugate and binding agent for use according to any of embodiments 9-27, wherein the payload of the linker-payload moiety of the antibody-drug conjugate comprises a structure shown as formula (III-A):

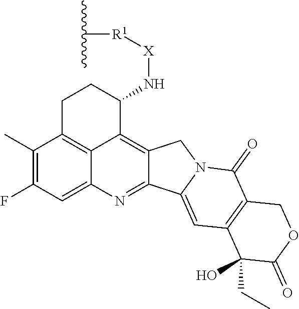

or a tautomer, mesomer, enantiomer thereof, or a pharmaceutically acceptable salt thereof,
wherein R¹ is selected from the group consisting of: —O—, —(R²)N—, —P(═O)(R²)— and —S—;
X is -L¹-CH₂—C(O)—;
L¹ is —(C(R^{3a})(R^{3b}))ₘ—, wherein 0 or at least 1 methylene unit of L¹ is independently replaced by —C(O)—, —C(═S)—, —C(═NR^{4b})— or —C(═N₂)—;
wherein each R², each R^{3a}, each R^{3b} and each R^{4b} are each independently hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group optionally substituted with R;
wherein each R, each R^{a} and each R^{b} are each independently hydrogen, protium, deuterium, tritium, halogen, —NO2, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C1-6 aliphatic group;
m is selected from the group consisting of integers ≥0,
when R¹ is —O— or —HN—, at least 1 methylene unit of L¹ is independently replaced by —C(O)—, —C(═S)—, —C(═NR^{4b})— or —C(═N₂)—, or each R^{3a} and each R^{3b} are not both hydrogen.

29. The combination according to any of embodiments 9-28, binding agent for use according to any of embodiments 9-28, antibody-drug conjugate for use according to any of embodiments 9-28, or antibody-drug conjugate and binding agent for use according to any of embodiments 9-28, wherein the linker-payload moiety of the antibody-drug conjugate comprises a structure shown as formula (III-C):

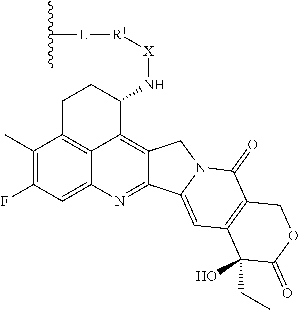

or a tautomer, mesomer, enantiomer thereof, or a pharmaceutically acceptable salt thereof wherein, L is -Lₐ-L_{b}-L_{c}-;
-Lₐ- is selected from the group consisting of:

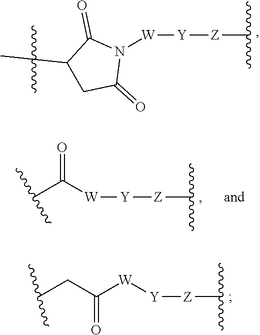

wherein W is —(C(R^{wa})(R^{wb}))_{wn}—, Y is —(OCH₂CH₂)_{yn}—O_{yp}, and Z is —(C(R^{za})(R^{zb}))_{zn};
wherein wn is selected from the group consisting of integers ≥0, and
0 or at least 1 methylene unit of W is independently replaced by -Cyr-, —N(R^{wx})C(O)—, —C(O)N(R^{wx})—, —C(O)—, —OC(O)—, —C(O)O—, —NR^{wx}—, —O—, —S—, —SO—, —SO₂—, —P(R^{wx})—, —P(═O)(R^{wx})—, —N(R^{wx})SO₂—, —SO₂N(R^{wx})—, —C(═S)—, —C(═NR^{wx})—, —N═N—, —C═N—, —N═C— or —C(═N₂)—;
wherein yn is selected from the group consisting of integers ≥0, and yp is 0 or 1;
wherein zn is selected from the group consisting of integers ≥0, and
0 or at least 1 methylene unit of Z is independently replaced by -Cyr-, —N(R^{zx})C(O)—, —C(O)N(R^{zx})—, —C(O)—, —OC(O)—, —C(O)O—, —NR^{x}—, —O—, —S—, —SO—, —SO₂—, —P(R^{zx})—, —P(═O)(R^{x})—N(R^{x})SO₂—, —SO₂N(R^{x})—, —C(═S)—, —C(═NR^{wx})—, —N═N—, —C═N—, —N═C— or —C(═N₂)—;
-Cyr- is selected from the group consisting of: 6-10 membered arylene, 5-8 membered heteroarylene, 3-10 membered heterocyclylene and 3-10 membered saturated or partially unsaturated carbocyclylene, wherein -Cyr- is unsubstituted or independently substituted with at least 1 substituent R^{cx};
wherein each R^{wa}, each R^{wb}, each R^{za}, each R^{zb}, each R^{wx}, each R^{zx} and each R^{cx} are each independently hydrogen, protium, deuterium, tritium, halogen, —NO₂, —CN, —OR^{r}, —SR^{r}, —N(R^{ra})(R^{rb}), —C(O)R^{r}, —C₂R^{r}, —C(O)C(O)R^{r}, —C(O)CH₂C(O)R^{r}, —S(O)R^{r}, —S(O)₂R^{r}, —C(O)N(R^{ra})(R^{rb}), —SO₂N(R^{ra})(R^{rb}), —OC(O)R^{r}, —N(R)SO₂R^{r}, or a C₁₋₆ aliphatic group optionally substituted with R^{r};
wherein each R^{r}, each R^{ra} and each R^{rb} are each independently hydrogen, protium, deuterium, tritium, halogen, —NO2, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
-L_{b}- represents a peptide residue consisting of 2 to 7 amino acids;
-L_{c}- is selected from the group consisting of:

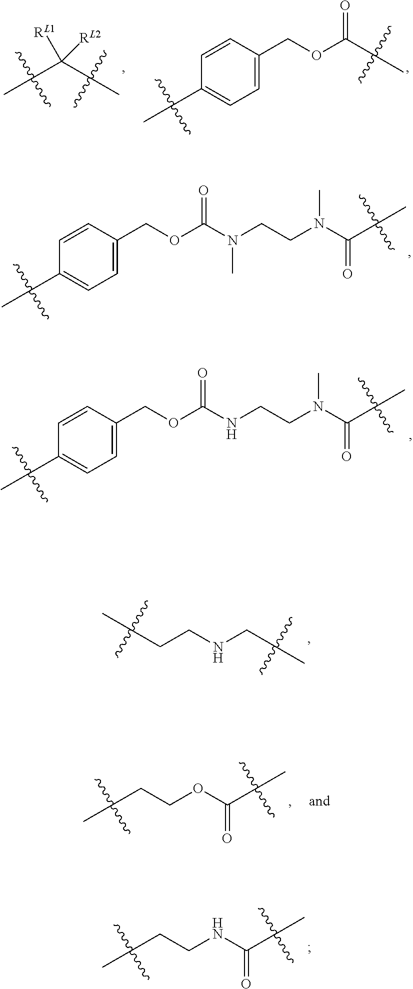

wherein R^{L1} and R^{L2} are each independently selected from the group consisting of hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H and a C₁₋₆ aliphatic group.

30. The combination according to embodiment 29, binding agent for use according to embodiment 29, antibody-drug conjugate for use according to embodiment 29, or antibody-drug conjugate and binding agent for use according to embodiment 29, wherein the antibody-drug conjugate comprises a structure shown as formula (III-D):

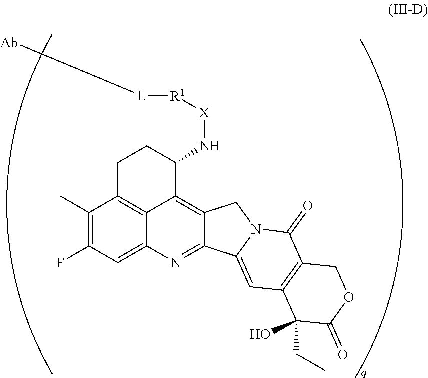

or a tautomer, mesomer, enantiomer thereof, or a pharmaceutically acceptable salt thereof wherein, Ab is an antibody or fragment thereof that specifically binds to a target expressed on the surface of a tumour cell, preferably an antibody or a fragment thereof that specifically binds to B7H3, and a connection number q is an integer from 1 to 10.

31. The combination according to embodiment 29, binding agent for use according to embodiment 29, antibody-drug conjugate for use according to embodiment 29, or antibody-drug conjugate and binding agent for use according to embodiment 29, wherein the antibody-drug conjugate comprises a structure shown as formula (III-D):

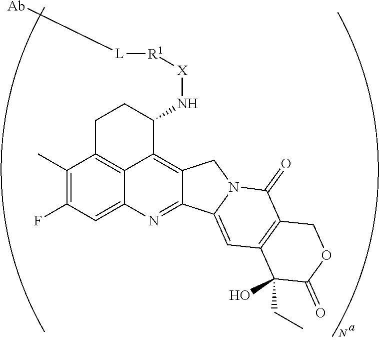

or a tautomer, mesomer, enantiomer thereof, or a pharmaceutically acceptable salt thereof wherein, Ab is an antibody or fragment thereof that specifically binds to a target expressed on the surface of a tumour cell, preferably an antibody or a fragment thereof that specifically binds to TROP2, and a connection number Na is an integer from 1 to 10.

32. The combination according to any of embodiments 26-27 or 29-31, binding agent for use according to any of embodiments 26-27 or 29-31, antibody-drug conjugate for use according to any of embodiments 26-27 or 29-31, or antibody-drug conjugate and binding agent for use according to any of embodiments 26-27 or 29-31, wherein,
-Lₐ- is

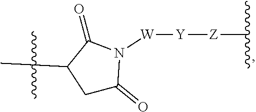

wherein W is —(C(R^{wa})(R^{wb}))_{wn}—, Y is —(OCH₂CH₂)_{yn}—O_{yp}—, and Z is —(C(R^{za})(R^{zb}))_{zn};
wherein wn is 1, 2, 3 or 6, and
1 methylene unit of W is independently replaced by -Cyr-, —N(R^{wx})C(O)—, —C(O)N(R^{wx})— or —C(O)—;
wherein yn is 0, 4 or 8, and yp is 0 or 1;
wherein zn is 1, 2 or 3, and
1 methylene unit of Z is independently replaced by -Cyr-, —N(R^{zx})C(O)—, —C(O)N(R^{zx})— or —C(O)—;
-Cyr- is 3-10 membered saturated carbocyclylene, wherein -Cyr- is unsubstituted or independently substituted with 1 to 3 substituent R^{cx};
wherein each R^{wa}, each R^{wb}, each R^{za}, each R^{zb}, each R^{wx}, each R^{zx} and each R^{cx} are each independently hydrogen, halogen, —OR^{r}, or a C₁₋₆ aliphatic group optionally substituted with R^{r};
wherein each R^{r} is independently hydrogen, halogen or a C₁₋₆ aliphatic group;
-L_{b}- is selected from the group consisting of

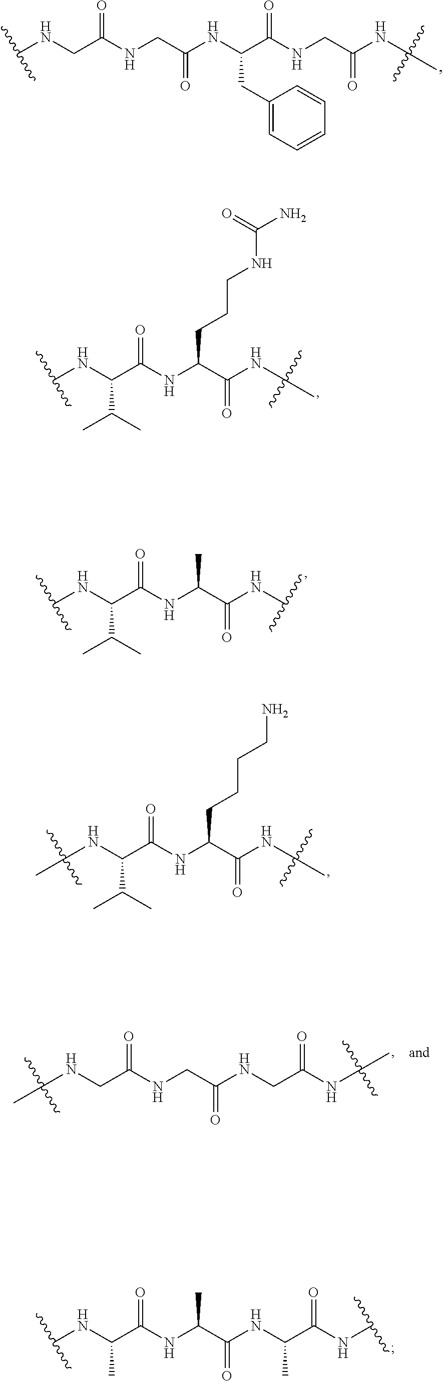 [SMILES: *NCC(=O)NCC(=O)N[C@@H](Cc1ccccc1)C(=O)NCC(=O)N* ; *NC(=O)[C@H](CCCNC(N)=O)NC(=O)[C@@H](N*)C(C)C ; *NC(=O)[C@H](C)NC(=O)[C@@H](N*)C(C)C ; *NC(=O)[C@H](CCCCN)NC(=O)[C@@H](N*)C(C)C ; *NCC(=O)NCC(=O)NCC(=O)N* ; *NC(=O)[C@H](C)NC(=O)[C@H](C)NC(=O)[C@H](C)N*]

-L_{c}- is

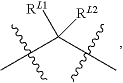

wherein R^{L1} and R^{L2} are each independently selected from the group consisting of hydrogen, halogen, —OH and a C1.6 aliphatic group.

33. The combination according to any of embodiments 26-27 or 29-32, binding agent for use according to any of embodiments 26-27 or 29-32, antibody-drug conjugate for use according to any of embodiments 26-27 or 29-32, or antibody-drug conjugate and binding agent for use according to any of embodiments 26-27 or 29-32, wherein,
-Lₐ- is

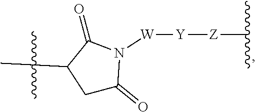

wherein W is —(C(R^{wa})(R^{wb}))_{wn}—, Y is —(OCH₂CH₂)_{yn}—Oyₚ-, and Z is —(C(R^{za})(R^{zb}))_{zn};
wherein wn is 1, 2, 3 or 6, and
1 methylene unit of W is independently replaced by -Cyr-, —N(R^{wx})C(O)—, —C(O)N(R^{wx})— or —C(O)—;
wherein yn is 0, 4 or 8, and yp is 0 or 1;
wherein zn is 1, 2 or 3, and
1 methylene unit of Z is independently replaced by -Cyr-, —N(R^{zx})C(O)—, —C(O)N(R^{zx})— or —C(O)—;
-Cyr- is 3-10 membered saturated carbocyclylene, wherein -Cyr- is unsubstituted or independently substituted with 1 to 3 substituent R^{cx};
wherein each R^{wa}, each R^{wb}, each R^{za}, each R^{zb}, each R^{wx}, each R^{zx} and each R^{cx} are each independently hydrogen, halogen, —OR^{r}, or a C₁₋₆ aliphatic group optionally substituted with R^{r};
wherein each R^{r} is independently hydrogen, halogen or a C₁₋₆ aliphatic group;
-L_{b}- is

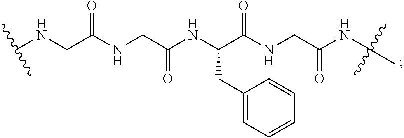 [SMILES: *NCC(=O)NCC(=O)N[C@@H](Cc1ccccc1)C(=O)NCC(=O)N*]

-L_{c}- is

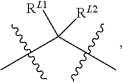

wherein R^{L1} and R^{L2} are each independently selected from the group consisting of hydrogen, halogen, —OH and a C₁₋₆ aliphatic group.

34. The combination according to any of embodiments 26-27 or 29-33, binding agent for use according to any of embodiments 26-27 or 29-33, antibody-drug conjugate for use according to any of embodiments 26-27 or 29-33, or antibody-drug conjugate and binding agent for use according to any of embodiments 26-27 or 29-33, wherein,
-Lₐ- is

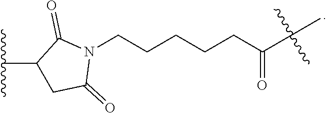 [SMILES: *C(=O)CCCCCN1C(=O)CC(*)C1=O]

35. The combination according to any of embodiments 26-27 or 29-34, binding agent for use according to any of embodiments 26-27 or 29-34, antibody-drug conjugate for use according to any of embodiments 26-27 or 29-34, or antibody-drug conjugate and binding agent for use according to any of embodiments 26-27 or 29-34, wherein,
-L_{b}- is

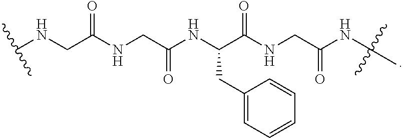 [SMILES: *NCC(=O)NCC(=O)N[C@@H](Cc1ccccc1)C(=O)NCC(=O)N*]

36. The combination according to any of embodiments 26-27 or 29-35, binding agent for use according to any of embodiments 26-27 or 29-35, antibody-drug conjugate for use according to any of embodiments 26-27 or 29-35, or antibody-drug conjugate and binding agent for use according to any of embodiments 26-27 or 29-35, wherein,
-L_{c}- is

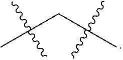 [SMILES: *C*]

37. The combination according to any of embodiments 26-27 or 29-36, binding agent for use according to any of embodiments 26-27 or 29-36, antibody-drug conjugate for use according to any of embodiments 26-27 or 29-36, or antibody-drug conjugate and binding agent for use according to any of embodiments 26-27 or 29-36, wherein,
-Lₐ-L_{b}-L_{c}- is

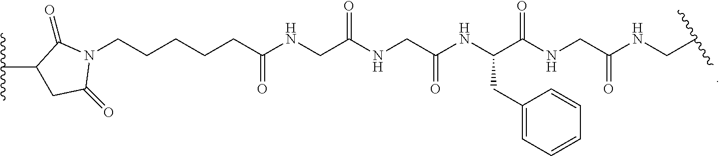 [SMILES: *CNC(=O)CNC(=O)[C@H](Cc1ccccc1)NC(=O)CNC(=O)CNC(=O)CCCCCN1C(=O)CC(*)C1=O]

38. The combination according to any of embodiments 9-37, binding agent for use according to any of embodiments 9-37, antibody-drug conjugate for use according to any of embodiments 9-37, or antibody-drug conjugate and binding agent for use according to any of embodiments 9-37, wherein the payload moiety of the antibody-drug conjugate comprises the following-group of structures:

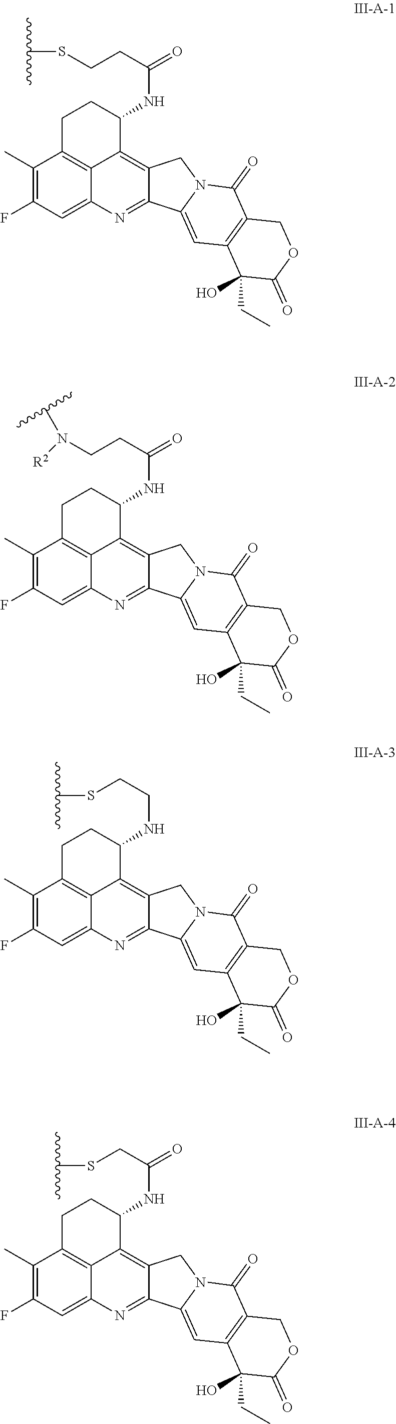

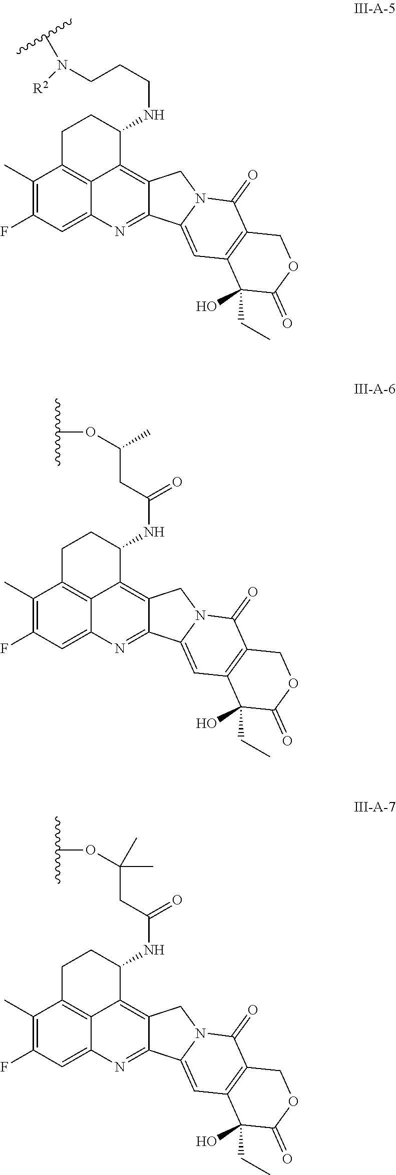

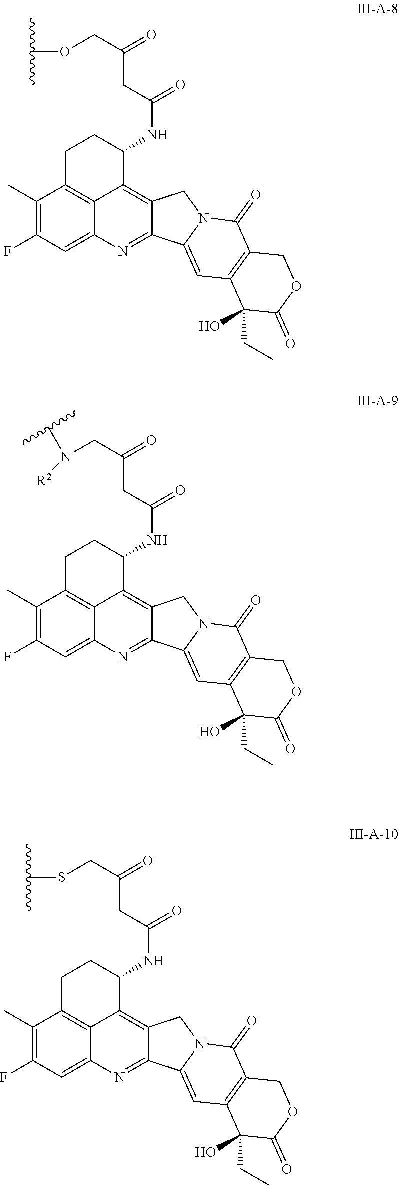

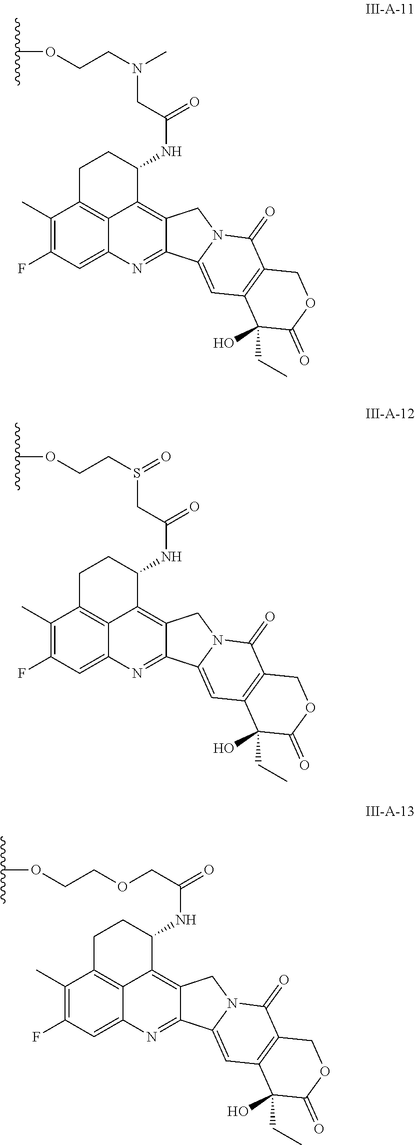 [SMILES: *OCCN(C)CC(=O)N[C@H]1CCc2c(C)c(F)cc3nc4c(c1c23)Cn1c-4cc2c(c1=O)COC(=O)[C@]2(O)CC ; *OCCS(=O)CC(=O)N[C@H]1CCc2c(C)c(F)cc3nc4c(c1c23)Cn1c-4cc2c(c1=O)COC(=O)[C@]2(O)CC ; *OCCOCC(=O)N[C@H]1CCc2c(C)c(F)cc3nc4c(c1c23)Cn1c-4cc2c(c1=O)COC(=O)[C@]2(O)CC]

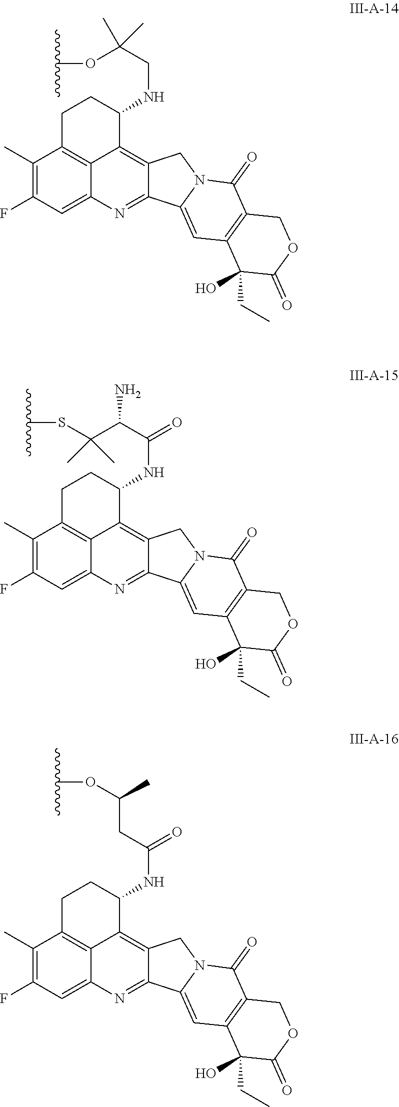 [SMILES: *OC(C)(C)CN[C@H]1CCc2c(C)c(F)cc3nc4c(c1c23)Cn1c-4cc2c(c1=O)COC(=O)[C@]2(O)CC ; *SC(C)(C)[C@H](N)C(=O)N[C@H]1CCc2c(C)c(F)cc3nc4c(c1c23)Cn1c-4cc2c(c1=O)COC(=O)[C@]2(O)CC ; *O[C@@H](C)CC(=O)N[C@H]1CCc2c(C)c(F)cc3nc4c(c1c23)Cn1c-4cc2c(c1=O)COC(=O)[C@]2(O)CC]

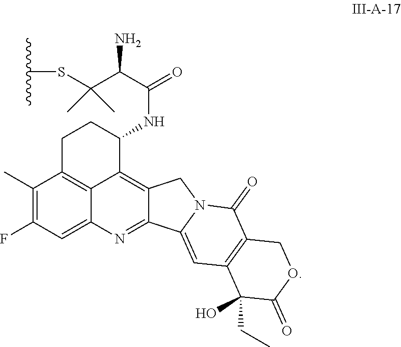 [SMILES: *SC(C)(C)[C@@H](N)C(=O)N[C@H]1CCc2c(C)c(F)cc3nc4c(c1c23)Cn1c-4cc2c(c1=O)COC(=O)[C@]2(O)CC]

39. The combination according to embodiment 38, binding agent for use according to embodiment 38, antibody-drug conjugate for use according to embodiment 38, or antibody-drug conjugate and binding agent for use according to embodiment 38, wherein the payload of the linker-payload moiety of the antibody-drug conjugate has the following structure:

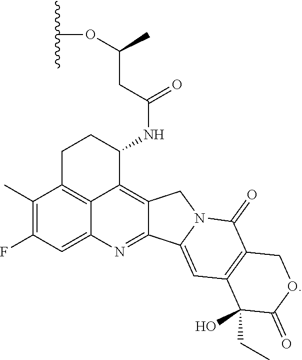 [SMILES: *O[C@@H](C)CC(=O)N[C@H]1CCc2c(C)c(F)cc3nc4c(c1c23)Cn1c-4cc2c(c1=O)COC(=O)[C@]2(O)CC]

40. The combination according to embodiment 39, binding agent for use according to embodiment 39, antibody-drug conjugate for use according to embodiment 39, or antibody-drug conjugate and binding agent for use according to embodiment 39, wherein the linker-payload moiety of the antibody-drug conjugate has the following structure:

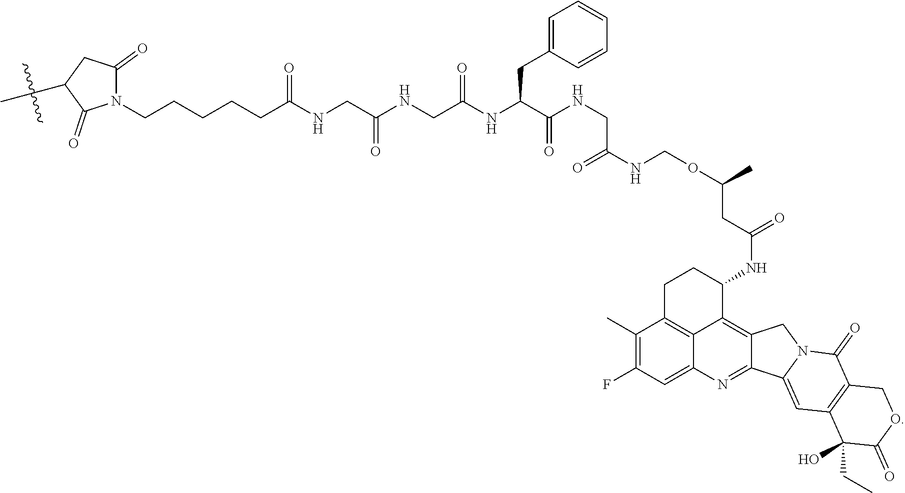 [SMILES: *C1CC(=O)N(CCCCCC(=O)NCC(=O)NCC(=O)N[C@@H](Cc2ccccc2)C(=O)NCC(=O)NCO[C@@H](C)CC(=O)N[C@H]2CCc3c(C)c(F)cc4nc5c(c2c34)Cn2c-5cc3c(c2=O)COC(=O)[C@]3(O)CC)C1=O]

41. The combination according to any one of embodiments 9-23, binding agent for use according to any one of embodiments 9-23, antibody-drug conjugate for use according to any one of embodiments 9-23, or antibody-drug conjugate and binding agent for use according to any one of embodiments 9-23, wherein the linker-payload moiety of the antibody-drug conjugate comprises a structure represented by formula:

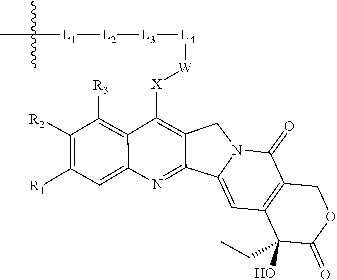

wherein,
L1 is selected from

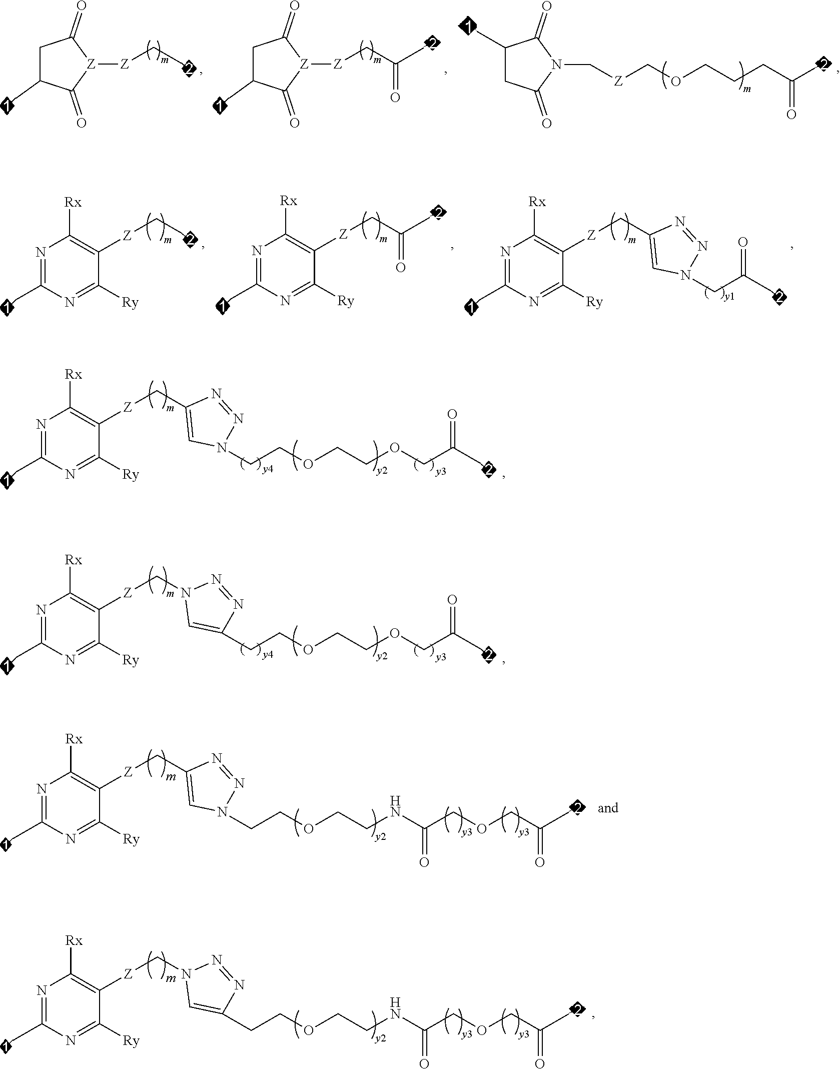

each Z is independently selected from a direct bond, a carbon-carbon triple bond, a carbon-carbon double bond, and amido (preferably selected from a direct bond, a carbon-carbon triple bond, and a carbon-carbon double bond);
Rx and Ry are each independently selected from H and C1-4 alkyl;
each m is independently selected from 0, 1, 2, 3, 4, 5 and 6;
y1 is selected from any integer between 1 and 6 (such as 4, 5, and 6);
each y2 is independently selected from any integer between 0 and 15 (such as 6-15);
each y3 is independently selected from 1, 2, and 3;
each y4 is independently selected from 0 and 1; position 1 is attached to Tb via an S atom, and position 2 is attached to L2 or L3;
L2 is absent or present, and when L2 is present, L2 is selected from:

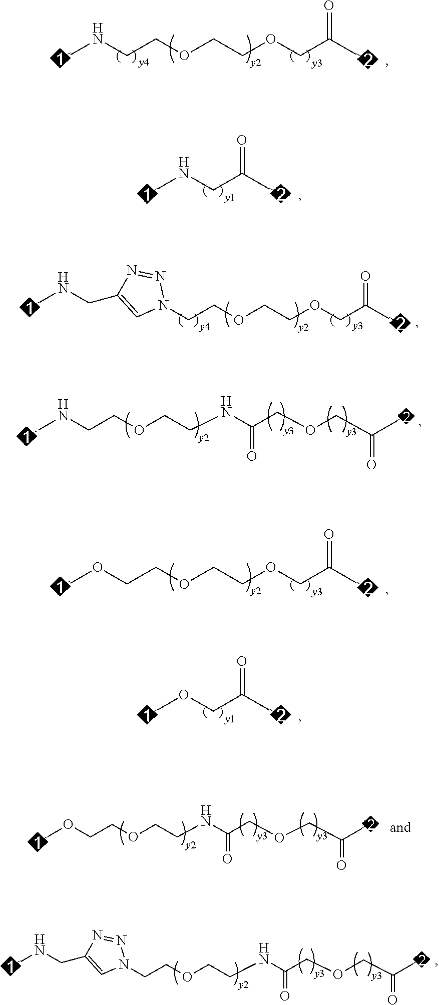

y1 is selected from any integer between 1 and 6 (such as 4, 5, and 6); each y2 is independently selected from any integer between 0 and 10 (such as 6-10); each y3 is independently selected from 1 or 2; each y4 is independently selected from 0 and 1, position 1 is attached to L1, and position 2 is attached to L3;
L3 is selected from an amino acid residue or a short peptide consisting of 2-10 amino acid residues; the amino acid residue is selected from natural amino acid residues, non-natural amino acid residues, or selected from amino acid residue represented by AA1 or stereoisomer thereof;

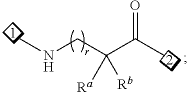

in the amino acid residue represented by AA1, any one of Ra and Rb is H, and the other is H

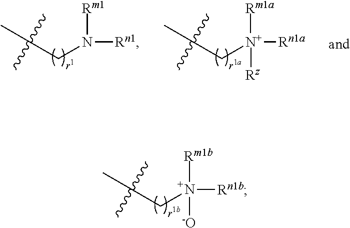

or, R^{a} and Rb, together with the carbon atom to which they are both attached, form a 5-6 membered heterocyclic ring, and said 5-6 membered heterocyclic ring is piperidine ring or piperazine ring.
r, r1, r1a and r1b are each independently 0, 1, 2, 3, 4 or 5;
Rm1, Rn1, Rm1a, Rn1a, Rm1b and Rn1b are each independently H, C1-6 alkyl or —COORx1, wherein, Rx1 is C1-6 alkyl;
or, Rm1 and Rn1, Rm1a and Rn1a, and Rm1b and Rn1b, together with the nitrogen atom to which they are both attached, form a 5-6 membered heterocyclic ring, and of said 5-6 membered heterocyclic ring, the heteroatom is selected from 1 or 2 N atoms; the said 5-6 membered heterocyclic ring is optionally substituted with one or more R0′;
Rz is selected from C1-6 alkyl;
R0 and R0′ are each independently selected from C1-6 alkyl, —NRm2Rn2 or 5-6 membered heterocyclyl optionally substituted with C1-6 alkyl; of said 5-6 membered heterocyclyl, the heteroatom is selected from 1 or 2 N atoms;
Rm2 and Rn2 are each independently selected from H and C1-6 alkyl;
L4 is absent or present, when L4 is present, L4 is selected from

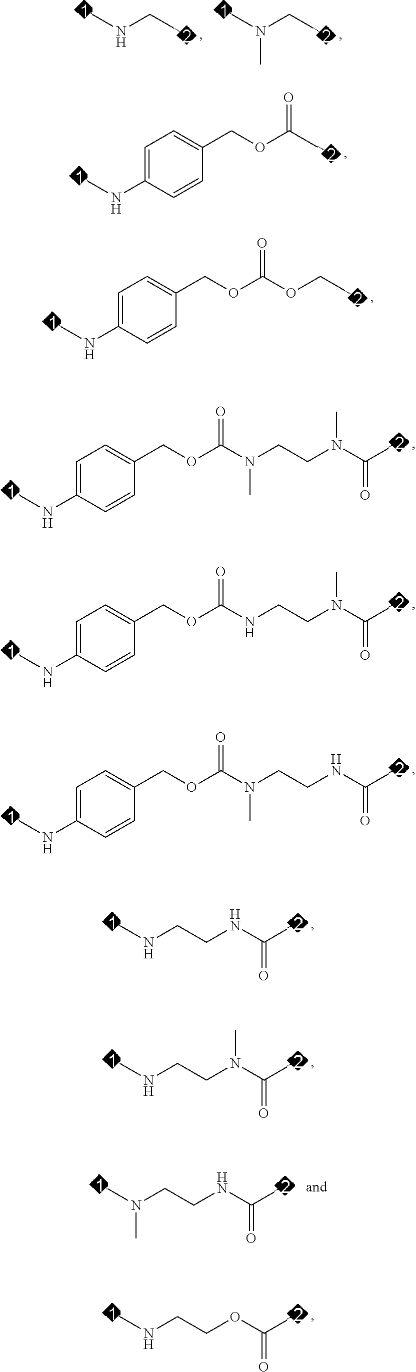

position 1 is attached to L3, and position 2 is attached to D;
R1 and R2 are each independently selected from H, halogens and C1-4 alkyl; or, R1 and R2, together with the carbon atom to which they are both attached form a 5-6 membered heterocyclic ring, the heterocyclic ring contains 1, 2, or 3 of O, S, or N or any combination thereof,
R3 is selected from H and C1-4 alkyl; or R3 and X, together with the carbon atom to which they are both attached, form a 5-6 membered carbon ring;
W is absent or present, when W is present, W is selected from —O—, —S—, —NR4-,

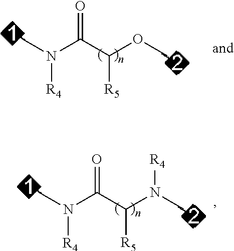

position 1 is attached to X, and position 2 is attached to L4 or L3;

X is selected from optionally substituted —(CH2)n1-,

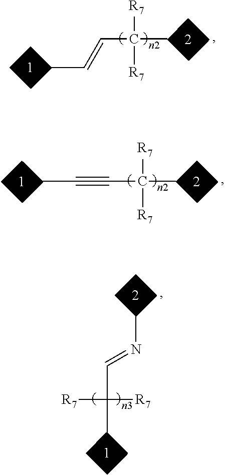

position 1 is attached to the parent ring and position 2 is attached to W or L4; the substituent is selected from one or two C1-4 alkyls;
R4, R5, and R7 are each independently selected from H and C1-4 alkyl;
n, n1, n2, n3 are each independently selected from any integer between 0 and 6.

42. The combination according to embodiment 41, binding agent for use according to embodiment 41, antibody-drug conjugate for use according to embodiment 41, or antibody-drug conjugate and binding agent for use according to embodiment 41, wherein the antibody-drug conjugate has a structure selected from:

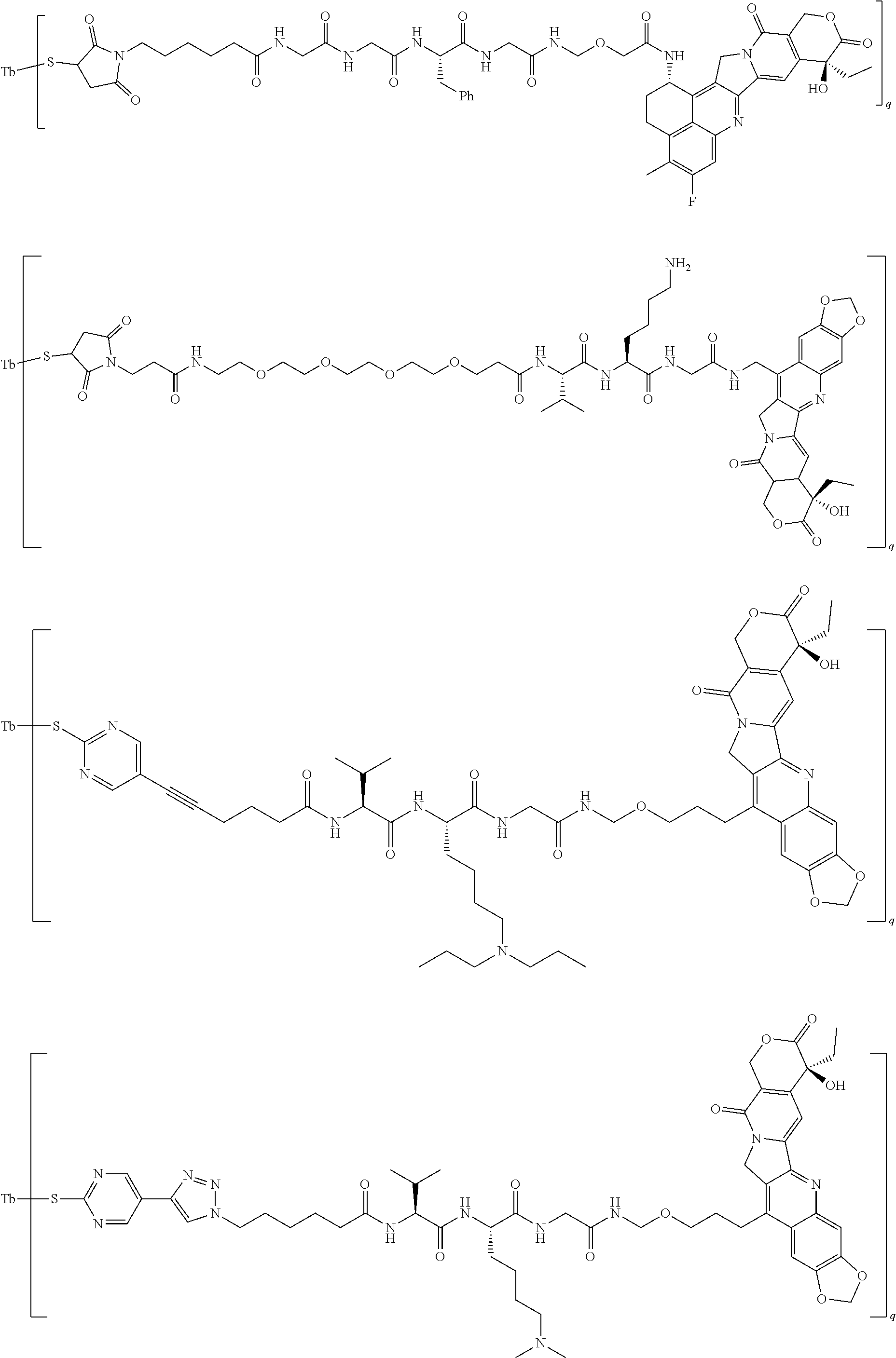

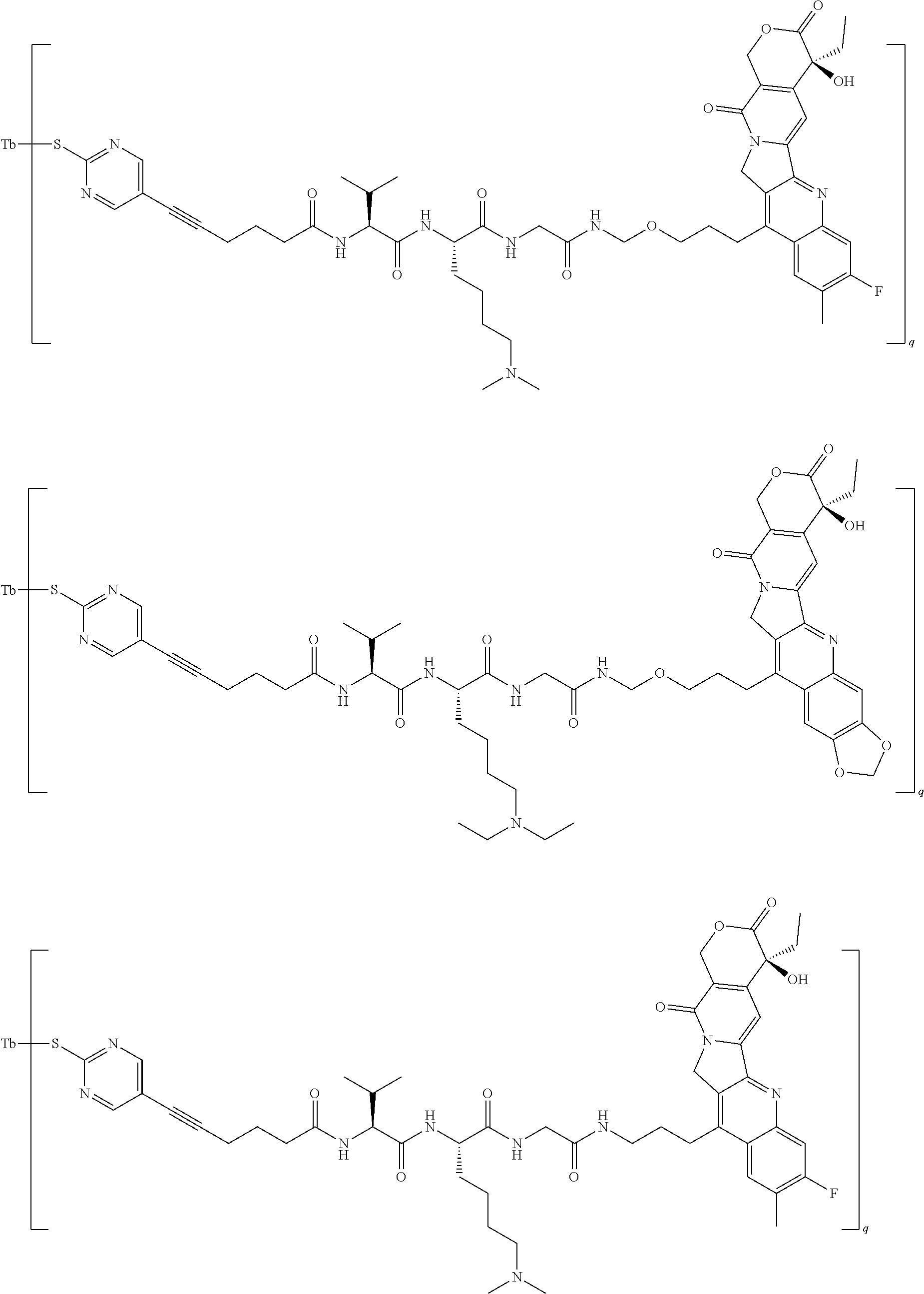

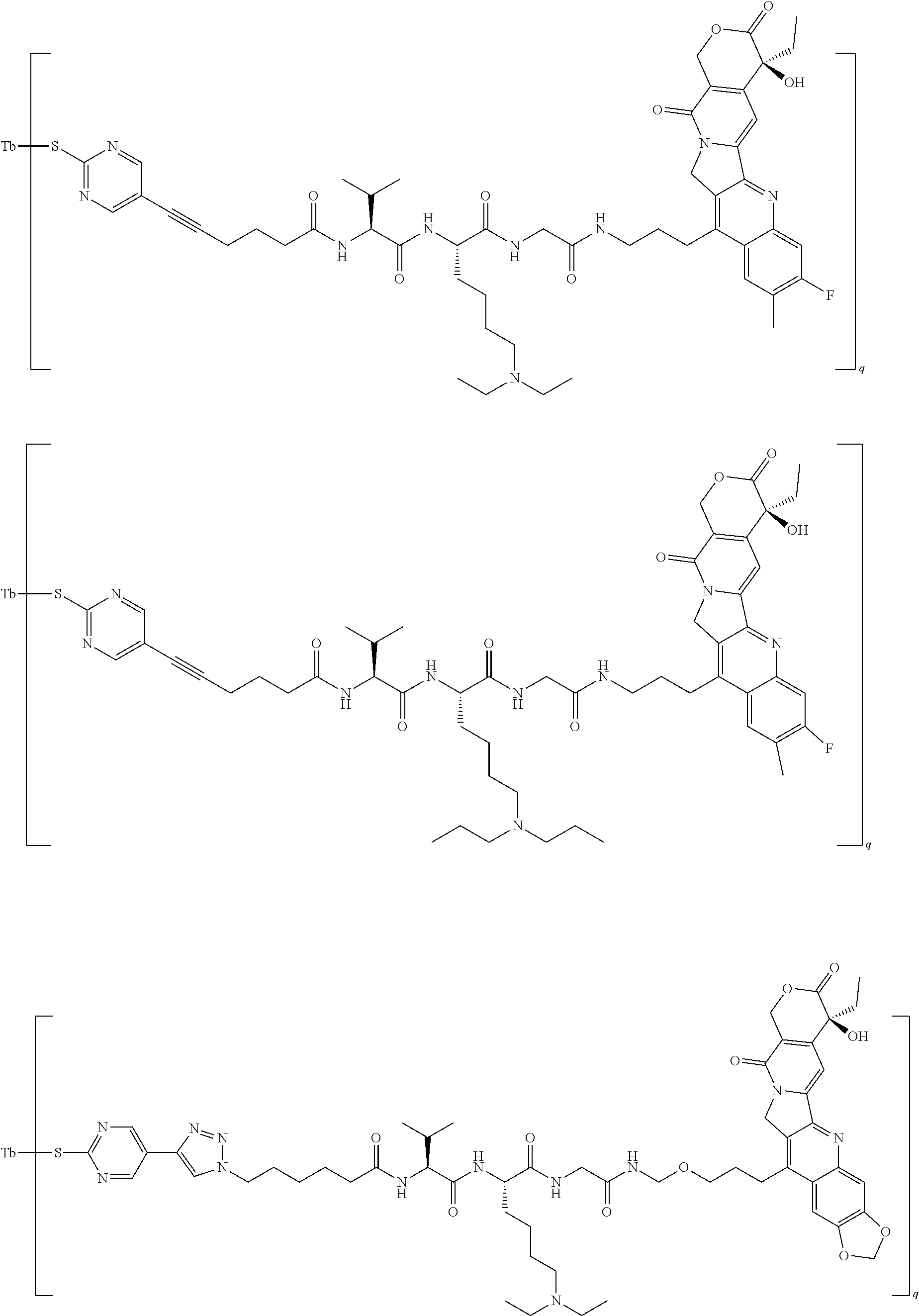

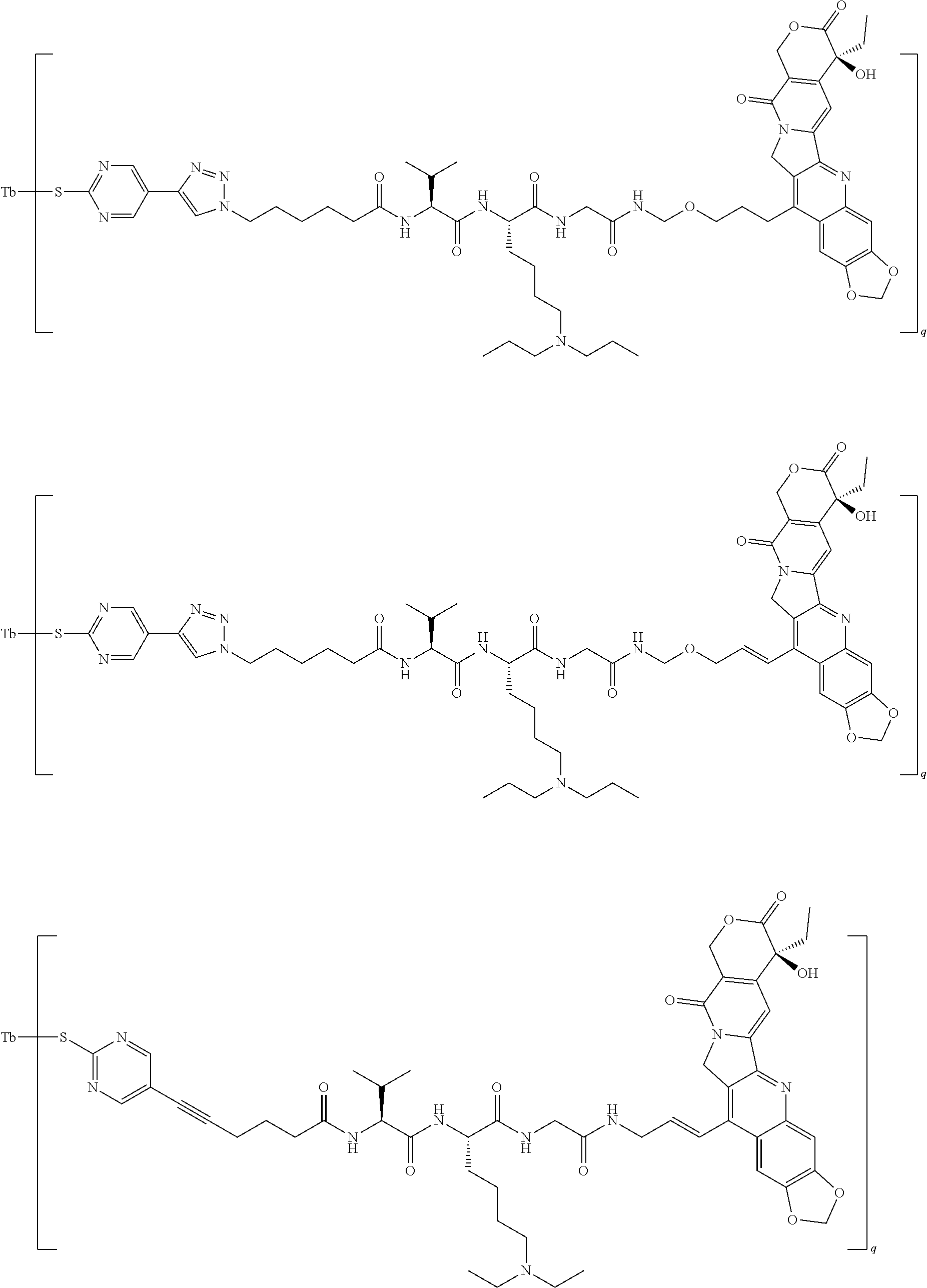

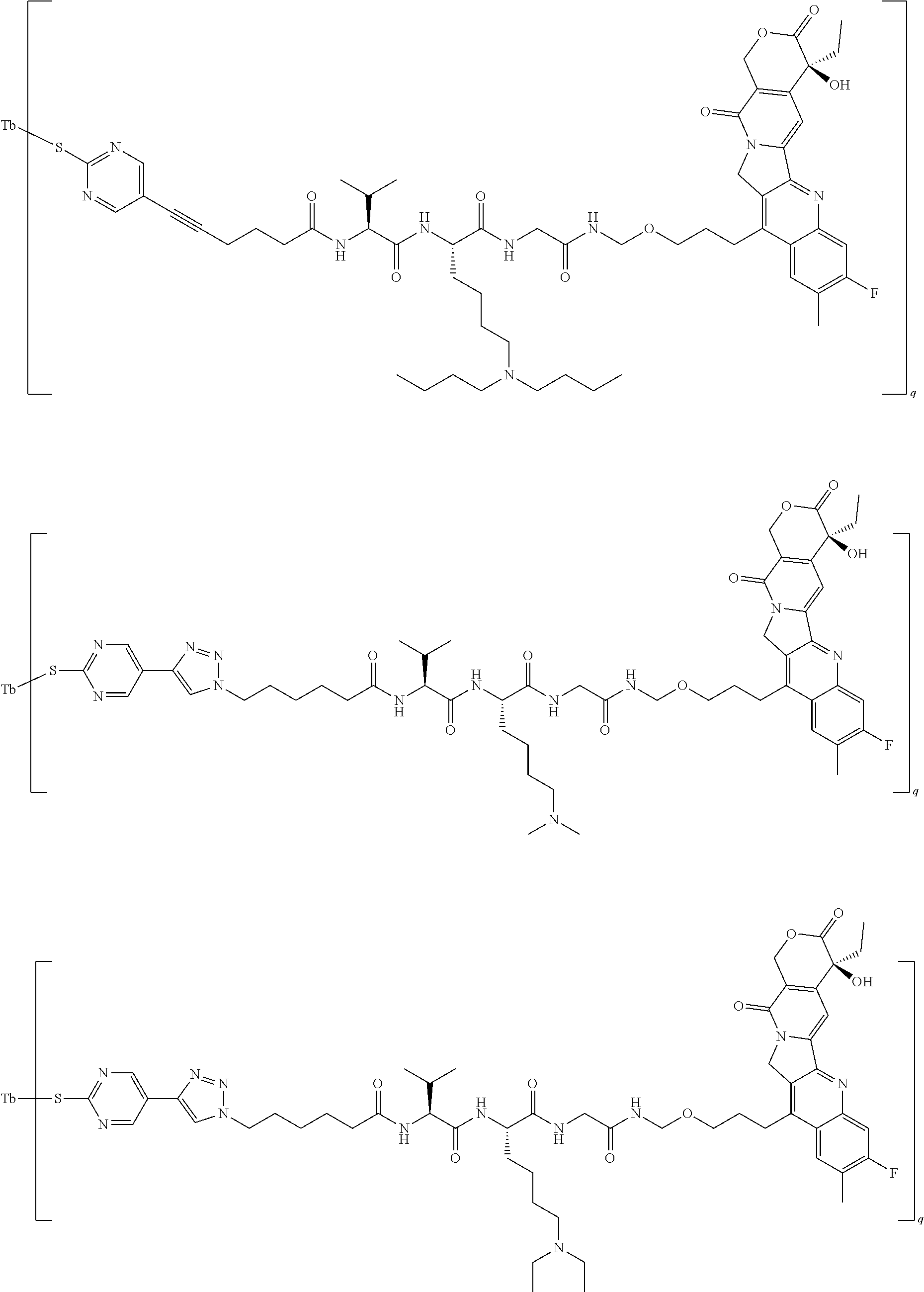

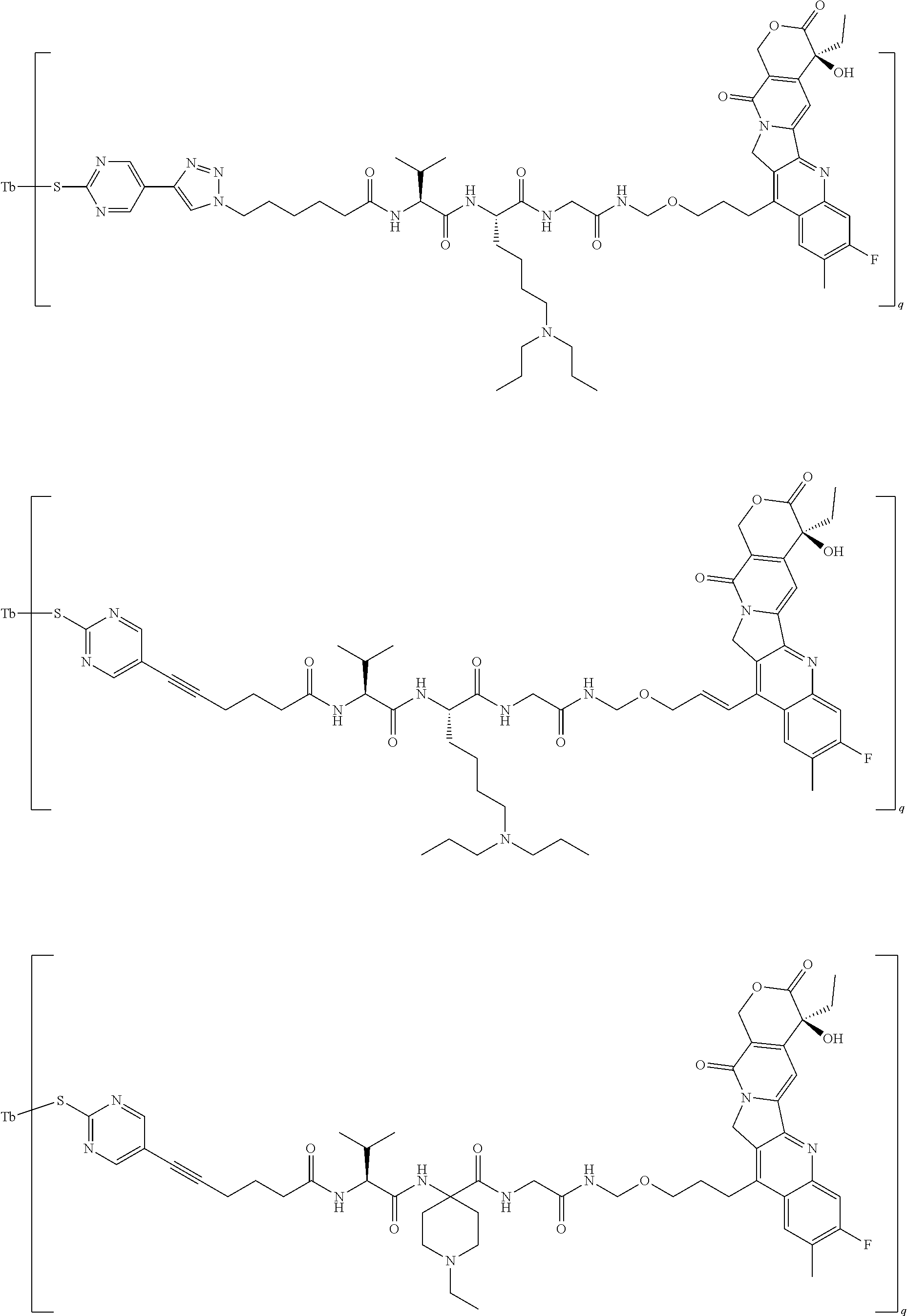

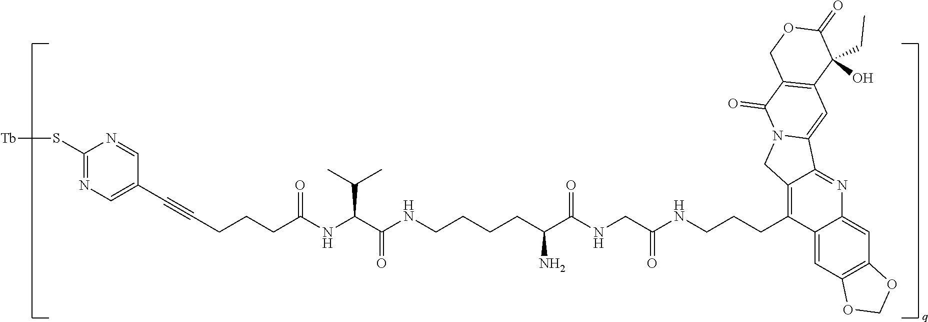

or a pharmaceutically acceptable salt thereof wherein:
Tb is an antibody or fragment thereof that specifically binds to a target expressed on the surface of a tumour cell; and
q is the connection number, and q is an integer from 1 to 16; preferably the connection number q is an integer from 4 to 12, more preferably q is an integer from 6 to 10; such as 7 to 9, such as 7, 8 or 9; preferably 8.

43. The combination according to embodiment 41, binding agent for use according to embodiment 41, antibody-drug conjugate for use according to embodiment 41, or antibody-drug conjugate and binding agent for use according to embodiment 41, wherein the antibody-drug conjugate has a structure selected from:

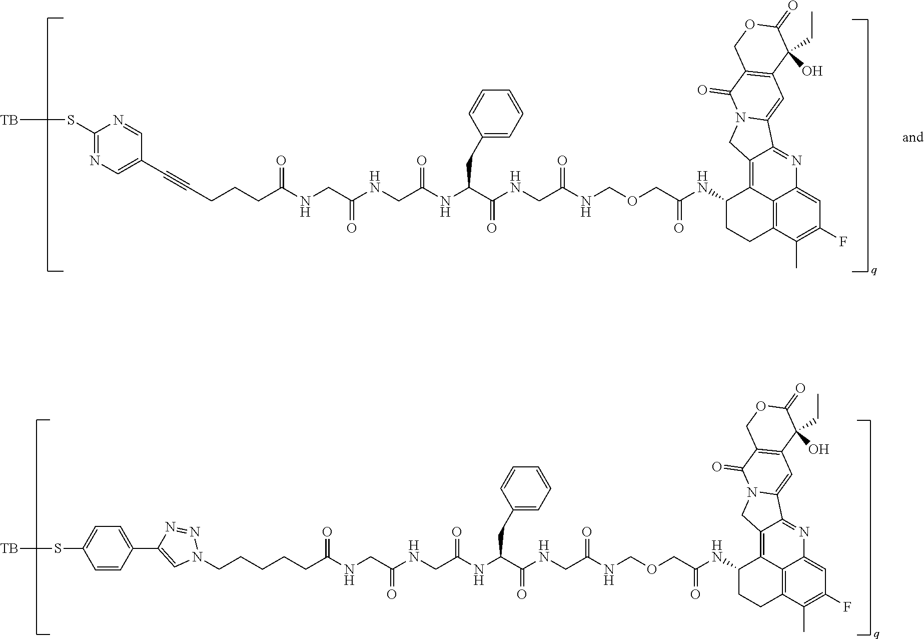

or a pharmaceutically acceptable salt thereof wherein:
Tb is an antibody or fragment thereof that specifically binds to a target expressed on the surface of a tumour cell; and
q is the connection number, and q is an integer from 1 to 16; preferably the connection number q is an integer from 4 to 12, more preferably q is an integer from 6 to 10; such as 7 to 9, such as 7, 8 or 9; preferably 8.

44. The combination according to embodiment 41, binding agent for use according to embodiment 41, antibody-drug conjugate for use according to embodiment 41, or antibody-drug conjugate and binding agent for use according to embodiment 41, wherein the antibody-drug conjugate has the structure (I-M)

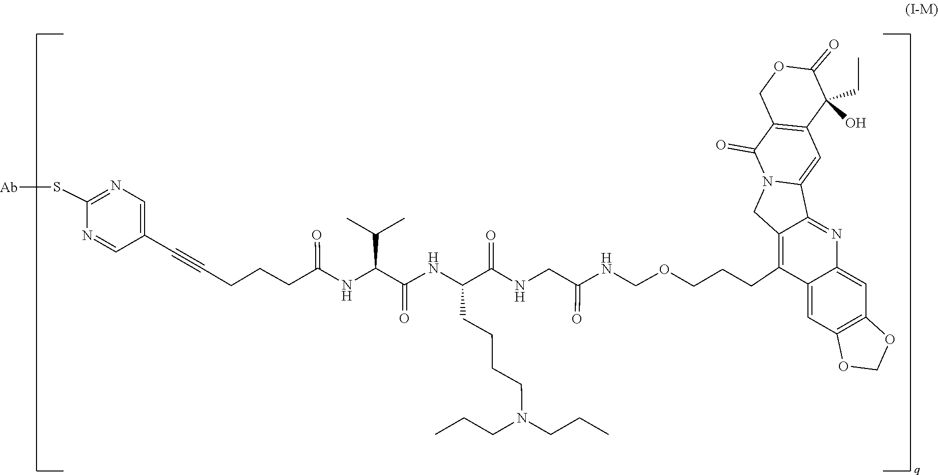

or a pharmaceutically acceptable salt thereof wherein:
Ab is an antibody or fragment thereof that specifically binds to a target expressed on the surface of a tumour cell; and
q is the connection number, and q is an integer from 1 to 16; preferably the connection number q is an integer from 4 to 12, more preferably q is an integer from 6 to 10; such as 7 to 9, such as 7, 8 or 9; preferably 8.

45. The combination according to any one of embodiments 1 or 5-44, binding agent for use according to any one of embodiments 2 or 5-44, antibody-drug conjugate for use according to any one of embodiments 3 or 5-44, or antibody-drug conjugate and binding agent for use according to any one of embodiments 4-44, wherein the antibody-drug conjugate comprises an anti-HER3 antibody or fragment thereof.

46. The combination according to embodiment 45, binding agent for use according to embodiment 45, antibody-drug conjugate for use according to embodiment 45, or antibody-drug conjugate and binding agent for use according to embodiment 45, wherein the antibody-drug conjugate comprises an anti-HER3 antibody or fragment thereof comprising:
(a) (i) a heavy chain variable region comprising: (i) a HCDR1 comprising or consisting of the amino acid sequence of SEQ ID NO: 190; (ii) a HCDR2 comprising or consisting of the amino acid sequence of SEQ ID NO: 191; and (iii) a HCDR3 comprising or consisting of the amino acid sequence of SEQ ID NO: 192; and a light chain variable region comprising: (i) a LCDR1 comprising or consisting of the amino acid sequence of SEQ ID NO: 193; (ii) a LCDR2 comprising or consisting of the amino acid sequence of SEQ ID NO: 194; and (iii) a LCDR3 comprising or consisting of the amino acid sequence of SEQ ID NO: 195, wherein the CDRs are defined according to the Kabat numbering system; and/or
(ii) a heavy chain variable region comprising: (i) a HCDR1 comprising or consisting of the amino acid sequence of SEQ ID NO: 196; (ii) a HCDR2 comprising or consisting of the amino acid sequence of SEQ ID NO: 197; and (iii) a HCDR3 comprising or consisting of the amino acid sequence of SEQ ID NO: 198; and a light chain variable region comprising: (i) a LCDR1 comprising or consisting of the amino acid sequence of SEQ ID NO: 199; (ii) a LCDR2 comprising or consisting of the amino acid sequence of SEQ ID NO: 200; and (iii) a LCDR3 comprising or consisting of the amino acid sequence of SEQ ID NO: 201, wherein the CDRs are defined according to the IMGT numbering system; or
(b) (i) a heavy chain variable region comprising: (i) a HCDR1 comprising or consisting of the amino acid sequence of SEQ ID NO: 206; (ii) a HCDR2 comprising or consisting of the amino acid sequence of SEQ ID NO: 207; and (iii) a HCDR3 comprising or consisting of the amino acid sequence of SEQ ID NO: 208; and a light chain variable region comprising: (i) a LCDR1 comprising or consisting of the amino acid sequence of SEQ ID NO: 209; (ii) a LCDR2 comprising or consisting of the amino acid sequence of SEQ ID NO: 210; and (iii) a LCDR3 comprising or consisting of the amino acid sequence of SEQ ID NO: 211, wherein the CDRs are defined according to the Kabat numbering system; and/or
(ii) a heavy chain variable region comprising: (i) a HCDR1 comprising or consisting of the amino acid sequence of SEQ ID NO: 212; (ii) a HCDR2 comprising or consisting of the amino acid sequence of SEQ ID NO: 213; and (iii) a HCDR3 comprising or consisting of the amino acid sequence of SEQ ID NO: 214; and a light chain variable region comprising: (i) a LCDR1 comprising or consisting of the amino acid sequence of SEQ ID NO: 215; (ii) a LCDR2 comprising or consisting of the amino acid sequence of SEQ ID NO: 216; and (iii) a LCDR3 comprising or consisting of the amino acid sequence of SEQ ID NO: 217, wherein the CDRs are defined according to the IMGT numbering system; or
(c) (i) a heavy chain variable region comprising: (i) a HCDR1 comprising or consisting of the amino acid sequence of SEQ ID NO: 220; (ii) a HCDR2 comprising or consisting of the amino acid sequence of SEQ ID NO: 221; and (iii) a HCDR3 comprising or consisting of the amino acid sequence of SEQ ID NO: 222; and a light chain variable region comprising: (i) a LCDR1 comprising or consisting of the amino acid sequence of SEQ ID NO: 223; (ii) a LCDR2 comprising or consisting of the amino acid sequence of SEQ ID NO: 224; and (iii) a LCDR3 comprising or consisting of the amino acid sequence of SEQ ID NO: 225, wherein the CDRs are defined according to the Kabat numbering system; and/or
(ii) a heavy chain variable region comprising: (i) a HCDR1 comprising or consisting of the amino acid sequence of SEQ ID NO: 226; (ii) a HCDR2 comprising or consisting of the amino acid sequence of SEQ ID NO: 227; and (iii) a HCDR3 comprising or consisting of the amino acid sequence of SEQ ID NO: 228; and a light chain variable region comprising: (i) a LCDR1 comprising or consisting of the amino acid sequence of SEQ ID NO: 229; (ii) a LCDR2 comprising or consisting of the amino acid sequence of SEQ ID NO: 230; and (iii) a LCDR3 comprising or consisting of the amino acid sequence of SEQ ID NO: 231, wherein the CDRs are defined according to the IMGT numbering system.

47. The combination according to embodiment 45 or 46, binding agent for use according to embodiment 45 or 46, antibody-drug conjugate for use according to embodiment 45 or 46, or antibody-drug conjugate and binding agent for use according to embodiment 45 or 46, wherein the antibody-drug conjugate comprises an anti-HER3 antibody or fragment thereof comprising:
(a) a heavy chain variable region comprising or consisting of the amino acid sequence of SEQ ID NO: 202 or an amino acid sequence having at least 80%, 85%, 90%, 95%, 96%, 97%, 98%, or 99% identity thereto; and a light chain variable region comprising or consisting of the amino acid sequence of SEQ ID NO: 203 or an amino acid sequence having at least 80%, 85%, 90%, 95%, 96%, 97%, 98%, or 99% identity thereto; or
(b) a heavy chain variable region comprising or consisting of the amino acid sequence of SEQ ID NO: 218 or an amino acid sequence having at least 80%, 85%, 90%, 95%, 96%, 97%, 98%, or 99% identity thereto; and a light chain variable region comprising or consisting of the amino acid sequence of SEQ ID NO: 219 or an amino acid sequence having at least 80%, 85%, 90%, 95%, 96%, 97%, 98%, or 99% identity thereto; or
(c) a heavy chain variable region comprising or consisting of the amino acid sequence of SEQ ID NO: 232 or an amino acid sequence having at least 80%, 85%, 90%, 95%, 96%, 97%, 98%, or 99% identity thereto; and a light chain variable region comprising or consisting of the amino acid sequence of SEQ ID NO: 233 or an amino acid sequence having at least 80%, 85%, 90%, 95%, 96%, 97%, 98%, or 99% identity thereto.

48. The combination according to any one of embodiments 45 to 47, binding agent for use according to any one of embodiments 45 to 47, antibody-drug conjugate for use according to any one of embodiments 45 to 47, or antibody-drug conjugate and binding agent for use according to any one of embodiments 45 to 47, wherein the antibody-drug conjugate comprises an anti-HER3 antibody or fragment thereof comprising a heavy chain comprising or consisting of the amino acid sequence of SEQ ID NO: 204 or an amino acid sequence having at least 80%, 85%, 90%, 95%, 96%, 97%, 98%, or 99% identity thereto; and a light chain comprising or consisting of the amino acid sequence of SEQ ID NO: 205 or an amino acid sequence having at least 80%, 85%, 90%, 95%, 96%, 97%, 98%, or 99% identity thereto.

49. The combination according to embodiment 48, binding agent for use according to embodiment 48, antibody-drug conjugate for use according to embodiment 48, or antibody-drug conjugate and binding agent for use according to embodiment 48, wherein the antibody-drug conjugate has the structure (I-M)

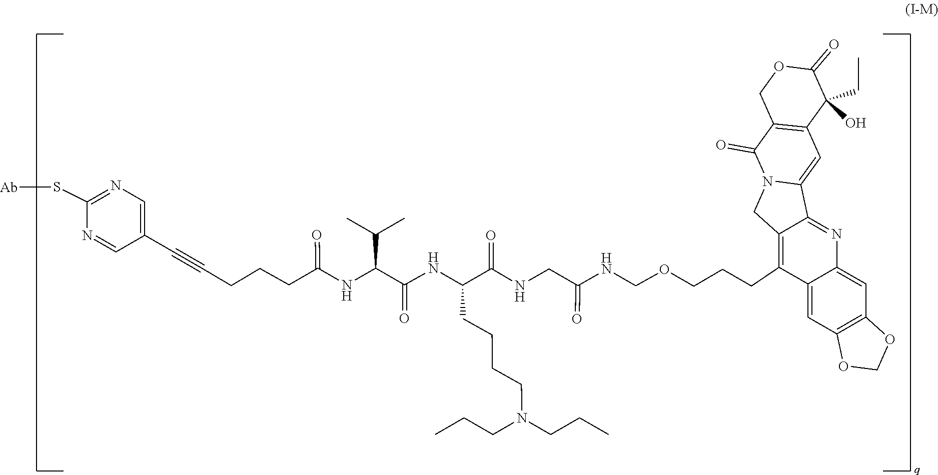

wherein,
Ab is an anti-HER3 antibody comprising a heavy chain amino acid sequence as set forth in SEQ ID NO: 204 and a light chain amino acid sequence as set forth in SEQ ID NO: 205; and q is the connection number, and q is an integer from 1 to 16; preferably the connection number q is an integer from 4 to 12, more preferably q is an integer from 6 to 10; such as 7 to 9, such as 7, 8 or 9; preferably 8.

50. The combination according to any of embodiments 1 or 5-43, binding agent for use according to any of embodiments 2 or 5-43, antibody-drug conjugate for use according to any of embodiments 3 or 5-43, or antibody-drug conjugate and binding agent for use according to any of embodiments 4 or 5-43, wherein the target expressed on the surface of a tumour cell is B7H3.

51. The combination according to any of embodiments 1 or 5-43, binding agent for use according to any of embodiments 2 or 5-43, antibody-drug conjugate for use according to any of embodiments 3 or 5-43, or antibody-drug conjugate and binding agent for use according to any of embodiments 4 or 5-43, wherein the antibody-drug conjugate comprises an anti-B7H3 antibody or fragment thereof.

52. The combination according to embodiment 50 or 51, binding agent for use according to embodiment 50 or 51, antibody-drug conjugate for use according to embodiment 50 or 51, or antibody-drug conjugate and binding agent for use according to embodiment 50 or 51, wherein the antibody-drug conjugate comprises an anti-B7H3 antibody or fragment thereof comprising:
(a) (i) a heavy chain variable region comprising: (i) a HCDR1 comprising or consisting of the amino acid sequence of SEQ ID NO: 1; (ii) a HCDR2 comprising or consisting of the amino acid sequence of SEQ ID NO: 2; and (iii) a HCDR3 comprising or consisting of the amino acid sequence of SEQ ID NO: 3; and a light chain variable region comprising: (i) a LCDR1 comprising or consisting of the amino acid sequence of SEQ ID NO: 4; (ii) a LCDR2 comprising or consisting of the amino acid sequence of SEQ ID NO: 5; and (iii) a LCDR3 comprising or consisting of the amino acid sequence of SEQ ID NO: 6, wherein the CDRs are defined according to the Kabat numbering system; and/or
(ii) a heavy chain variable region comprising: (i) a HCDR1 comprising or consisting of the amino acid sequence of SEQ ID NO: 7; (ii) a HCDR2 comprising or consisting of the amino acid sequence of SEQ ID NO: 8; and (iii) a HCDR3 comprising or consisting of the amino acid sequence of SEQ ID NO: 9; and a light chain variable region comprising: (i) a LCDR1 comprising or consisting of the amino acid sequence of SEQ ID NO: 10; (ii) a LCDR2 comprising or consisting of the amino acid sequence of SEQ ID NO: 11; and (iii) a LCDR3 comprising or consisting of the amino acid sequence of SEQ ID NO: 12, wherein the CDRs are defined according to the IMGT numbering system; or
(b) (i) a heavy chain variable region comprising: (i) a HCDR1 comprising or consisting of the amino acid sequence of SEQ ID NO: 17; (ii) a HCDR2 comprising or consisting of the amino acid sequence of SEQ ID NO: 18; and (iii) a HCDR3 comprising or consisting of the amino acid sequence of SEQ ID NO: 19; and a light chain variable region comprising: (i) a LCDR1 comprising or consisting of the amino acid sequence of SEQ ID NO: 20; (ii) a LCDR2 comprising or consisting of the amino acid sequence of SEQ ID NO: 21; and (iii) a LCDR3 comprising or consisting of the amino acid sequence of SEQ ID NO: 22, wherein the CDRs are defined according to the Kabat numbering system; and/or
(ii) a heavy chain variable region comprising: (i) a HCDR1 comprising or consisting of the amino acid sequence of SEQ ID NO: 23; (ii) a HCDR2 comprising or consisting of the amino acid sequence of SEQ ID NO: 24; and (iii) a HCDR3 comprising or consisting of the amino acid sequence of SEQ ID NO: 25; and a light chain variable region comprising: (i) a LCDR1 comprising or consisting of the amino acid sequence of SEQ ID NO: 26; (ii) a LCDR2 comprising or consisting of the amino acid sequence of SEQ ID NO: 27; and (iii) a LCDR3 comprising or consisting of the amino acid sequence of SEQ ID NO: 28, wherein the CDRs are defined according to the IMGT numbering system.

53. The combination according to any one of embodiments 50-52, binding agent for use according to any one of embodiments 50-52, antibody-drug conjugate for use according to any one of embodiments 50-52, or antibody-drug conjugate and binding agent for use according to any one of embodiments 50-52, wherein the antibody-drug conjugate comprises an anti-B7H3 antibody or fragment thereof comprising:
(a) a heavy chain variable region comprising or consisting of the amino acid sequence of SEQ ID NO: 13 or an amino acid sequence having at least 80%, 85%, 90%, 95%, 96%, 97%, 98%, or 99% identity thereto; and a light chain variable region comprising or consisting of the amino acid sequence of SEQ ID NO: 14 or an amino acid sequence having at least 80%, 85%, 90%, 95%, 96%, 97%, 98%, or 99% identity thereto; or
(b) a heavy chain variable region comprising or consisting of the amino acid sequence of SEQ ID NO: 29 or an amino acid sequence having at least 80%, 85%, 90%, 95%, 96%, 97%, 98%, or 99% identity thereto; and a light chain variable region comprising or consisting of the amino acid sequence of SEQ ID NO: 30 or an amino acid sequence having at least 80%, 85%, 90%, 95%, 96%, 97%, 98%, or 99% identity thereto. 54. The combination according to any of embodiments 50-53, binding agent for use according to any of embodiments 50-53, antibody-drug conjugate for use according to any of embodiments 50-53, or antibody-drug conjugate and binding agent for use according to any of embodiments 50-53, wherein the antibody-drug conjugate comprises an anti-B7H3 antibody or fragment thereof comprising:
(a) a heavy chain comprising or consisting of the amino acid sequence of SEQ ID NO: 15 or an amino acid sequence having at least 80%, 85%, 90%, 95%, 96%, 97%, 98%, or 99% identity thereto; and a light chain comprising or consisting of the amino acid sequence of SEQ ID NO: 16 or an amino acid sequence having at least 80%, 85%, 90%, 95%, 96%, 97%, 98%, or 99% identity thereto; or
(b) a heavy chain comprising or consisting of the amino acid sequence of SEQ ID NO: 31 or an amino acid sequence having at least 80%, 85%, 90%, 95%, 96%, 97%, 98%, or 99% identity thereto; and a light chain comprising or consisting of the amino acid sequence of SEQ ID NO: 32 or an amino acid sequence having at least 80%, 85%, 90%, 95%, 96%, 97%, 98%, or 99% identity thereto.

55. The combination according to embodiment 54, binding agent for use according to embodiment 54, antibody-drug conjugate for use according to embodiment 54, or antibody-drug conjugate and binding agent for use according to embodiment 54, wherein the antibody-drug conjugate has the structure (III-W):

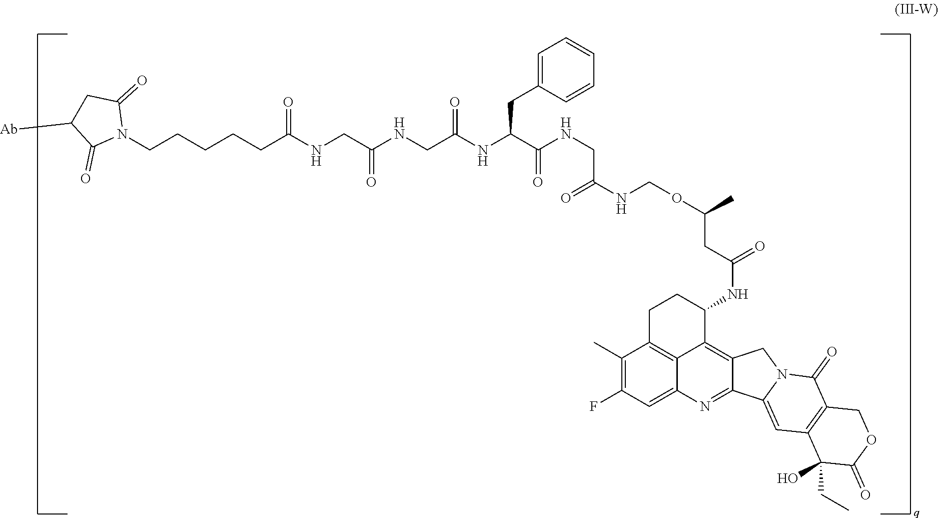

wherein,
q represents a connection number, and q is selected from the group consisting of integers from 1 to 10, preferably integers from 2 to 8; for example, q is an integer from 4 to 8, for example, q is an integer from 5 to 7, preferably 6; and
Ab is an anti-B7H3 antibody comprising a heavy chain amino acid sequence as set forth in SEQ ID NO: 31 and a light chain amino acid sequence as set forth in SEQ ID NO: 32.

56. The combination according to any one of embodiments 1 or 5-43, binding agent for use according to any one of embodiments 2 or 5-43, antibody-drug conjugate for use according to any one of embodiments 3 or 5-43, or antibody-drug conjugate and binding agent for use according to any one of embodiments 4 or 5-43, wherein the target expressed on the surface of a tumour cell is TROP2.

57. The combination according to any one of embodiments 1 or 5-43 or 56, binding agent for use according to any one of embodiments 2 or 5-43 or 56, antibody-drug conjugate for use according to any one of embodiments 3 or 5-43 or 56, or antibody-drug conjugate and binding agent for use according to any one of embodiments 4 or 5-43 or 56, wherein the antibody-drug conjugate comprises an anti-TROP2 antibody or fragment thereof.

58. The combination according to any one of embodiments 1 or 5-43 or 56-57, binding agent for use according to any one of embodiments 2 or 5-43 or 56-57, antibody-drug conjugate for use according to any one of embodiments 3 or 5-43 or 56-67, or antibody-drug conjugate and binding agent for use according to any one of embodiments 4-40 or 56-67, wherein the antibody-drug conjugate comprises Sacituzumab or a fragment thereof.

59. The combination according to any one of embodiments 56-58, binding agent for use according to any one of embodiments 56-58, antibody-drug conjugate for use according to any one of embodiments 56-58, or antibody-drug conjugate and binding agent for use according to any one of embodiments 56-58, wherein the antibody-drug conjugate comprises an anti-TROP2 antibody or fragment thereof comprising:
(i) a heavy chain variable region comprising: (i) a HCDR1 comprising or consisting of the amino acid sequence of SEQ ID NO: 33; (ii) a HCDR2 comprising or consisting of the amino acid sequence of SEQ ID NO: 34; and (iii) a HCDR3 comprising or consisting of the amino acid sequence of SEQ ID NO: 35; and a light chain variable region comprising: (i) a LCDR1 comprising or consisting of the amino acid sequence of SEQ ID NO: 36; (ii) a LCDR2 comprising or consisting of the amino acid sequence of SEQ ID NO: 37; and (iii) a LCDR3 comprising or consisting of the amino acid sequence of SEQ ID NO: 38, wherein the CDRs are defined according to the Kabat numbering system; and/or
(ii) a heavy chain variable region comprising: (i) a HCDR1 comprising or consisting of the amino acid sequence of SEQ ID NO: 39; (ii) a HCDR2 comprising or consisting of the amino acid sequence of SEQ ID NO: 40; and (iii) a HCDR3 comprising or consisting of the amino acid sequence of SEQ ID NO: 41; and a light chain variable region comprising: (i) a LCDR1 comprising or consisting of the amino acid sequence of SEQ ID NO: 42; (ii) a LCDR2 comprising or consisting of the amino acid sequence of SEQ ID NO: 43; and (iii) a LCDR3 comprising or consisting of the amino acid sequence of SEQ ID NO: 44, wherein the CDRs are defined according to the IMGT numbering system.

60. The combination according to any one of embodiments 56-59, binding agent for use according to any one of embodiments 56-59, antibody-drug conjugate for use according to any one of embodiments 56-59, or antibody-drug conjugate and binding agent for use according to any of embodiments 56-59, wherein the antibody-drug conjugate comprises an anti-TROP2 antibody or fragment thereof comprising a heavy chain variable region comprising or consisting of the amino acid sequence of SEQ ID NO: 45 or an amino acid sequence having at least 80%, 85%, 90%, 95%, 96%, 97%, 98%, or 99% identity thereto; and a light chain variable region comprising or consisting of the amino acid sequence of SEQ ID NO: 46 or an amino acid sequence having at least 80%, 85%, 90%, 95%, 96%, 97%, 98%, or 99% identity thereto.

61. The combination according to any one of embodiments 56-60, binding agent for use according to any one of embodiments 56-60, antibody-drug conjugate for use according to any one of embodiments 56-60, or antibody-drug conjugate and binding agent for use according to any one of embodiments 56-60, wherein the antibody-drug conjugate comprises an anti-TROP2 antibody or fragment thereof comprising a heavy chain comprising or consisting of the amino acid sequence of SEQ ID NO: 47 or an amino acid sequence having at least 80%, 85%, 90%, 95%, 96%, 97%, 98%, or 99% identity thereto; and a light chain comprising or consisting of the amino acid sequence of SEQ ID NO: 48 or an amino acid sequence having at least 80%, 85%, 90%, 95%, 96%, 97%, 98%, or 99% identity thereto.

62. The combination according to any one of embodiments 56-61, binding agent for use according to any one of embodiments 56-61, antibody-drug conjugate for use according to any one of embodiments 56-61-52, or antibody-drug conjugate and binding agent for use according to any one of embodiments 56-61, wherein the antibody-drug conjugate has the structure (III-S):

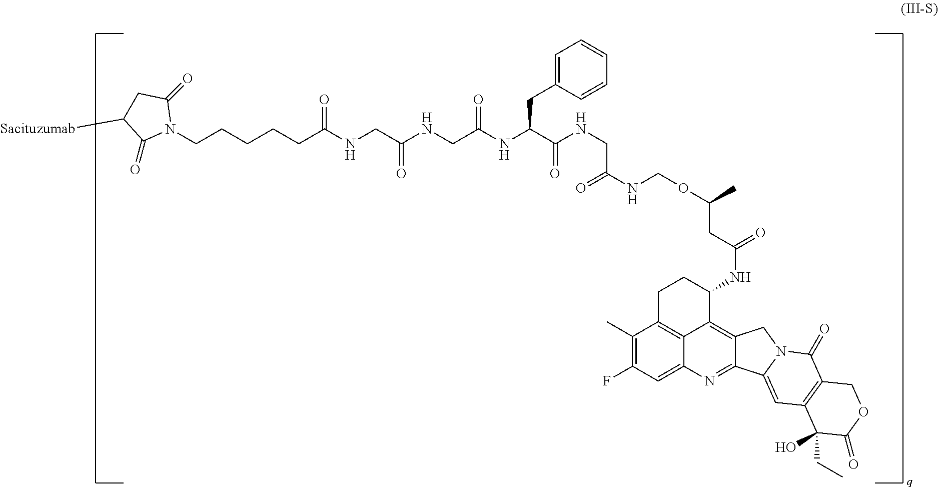

wherein,
q represents a connection number, and q is selected from the group consisting of integers from 1 to 8, preferably integers from 2 to 6, more preferably integers from 3 to 5, for example, preferably 3, 4 or 5; and
Sacituzumab is an anti-TROP2 antibody comprising a heavy chain amino acid sequence as set forth in SEQ ID NO: 47 and a light chain amino acid sequence as set forth in SEQ ID NO: 48.

63. The combination according to any one of embodiments 9-21, 45-48, 50-54 or 56-61, binding agent for use according to any one of embodiments 9-21, 45-48, 50-54 or 56-61, antibody-drug conjugate for use according to any one of embodiments 9-21, 45-48, 50-54 or 56-61, or antibody-drug conjugate and binding agent for use according to any one of embodiments 9-21, 45-48, 50-54 or 56-61, wherein the payload moiety of the antibody-drug conjugate has a structure shown as formula (II-A):

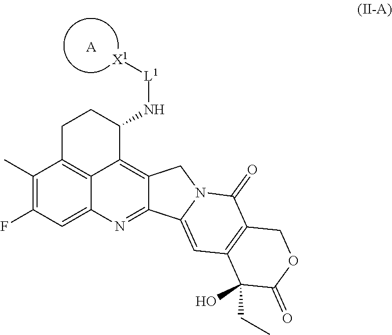

or a tautomer, mesomer, enantiomer thereof, or a pharmaceutically acceptable salt thereof wherein, X¹ is saturated C, and X¹ is substituted with Rⁿ;
ring A is selected from the group consisting of: 3-10 membered saturated or partially unsaturated heterocyclyl and 3-10 membered saturated or partially unsaturated carbocyclyl, wherein ring A is substituted with 0 or at least 1 substituent Ria;
when ring A is 3-10 membered saturated or partially unsaturated carbocyclyl, ring A is substituted with p L², and L² is not Rⁿ;
or, when ring A is 3-10 membered saturated or partially unsaturated heterocyclyl, ring A is substituted with p L2
L² is —R²-L³-, and R² is used for direct or indirect linking of a ligand;
L³ is —(C(R^{3a})(R^{3b}))ₘ—, wherein when L³ comprises a methylene unit, 0 or at least 1 methylene unit of L³ is independently replaced by —N(R⁴)C(O)—, —C(O)N(R⁴)—, —C(O)—, —OC(O)—, —C(O)O—, —NR⁴—, —O—, —S—, —SO—, —SO₂—, —P(R⁴)—, —P(═O)(R⁴)—, —N(R⁴)SO₂—, —SO₂N(R⁴)—, —C(═S)—, —C(═NR⁴)—, —N═N—, —C═N—, —N═C— or —C(═N₂)—;
R² is selected from the group consisting of: —O—, —(R^{2a})N—, —S— and —P(═O)(R^{2a})—;
L¹ is —(C(R^{5a})(R^{5b}))ₙ—, wherein when L¹ comprises a methylene unit, 0 or at least 1 methylene unit of L¹ is independently replaced by —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—;
wherein each Ria, each R^{2a}, each R^{3a}, each R^{3b}, each R⁴, each R^{5a}, each R^{5b}, each R⁶ and each Rⁿ are each independently hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group optionally substituted with R;
wherein each R, each R^{a} and each R^{b} are each independently hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C1.6 aliphatic group;
m and n are each independently selected from the group consisting of integers ≥0, and p is an integer ≥1.

64. The combination according to embodiment 63, binding agent for use according to embodiment 63, antibody-drug conjugate for use according to embodiment 63, or antibody-drug conjugate and binding agent for use according to embodiment 63, wherein the payload-linker moiety of the antibody-drug conjugate has a structure shown as formula (II-Cₓ):

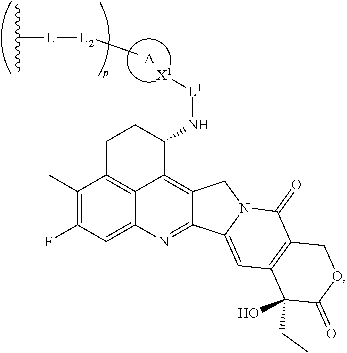

or a tautomer, mesomer, enantiomer thereof, or a pharmaceutically acceptable salt thereof wherein, L is -Lₐ-L_{b}-L_{c}-;
-Lₐ- is selected from the group consisting of:

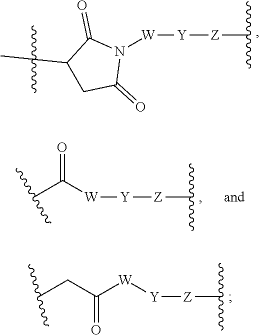

wherein W is —(C(R^{wa})(R^{wb}))_{wn}—, Y is —(OCH₂CH₂)_{yn}—O_{yp}—, and Z is —(C(R^{za})(R^{zb}))zn; wherein wn is selected from the group consisting of integers ≥0, and
0 or at least 1 methylene unit of W is independently replaced by -Cyr-, —N(R^{wx})C(O)—, —C(O)N(R^{wx})—, —C(O)—, —OC(O)—, —C(O)O—, —NR^{wx}—, —O—, —S—, —SO—, —SO2-, —P(R^{wx})—, —P(═O)(R^{wx})—, —N(R^{wx})SO₂—, —SO₂N(R^{wx})—, —C(═S)—, —C(═NR^{wx})—, —N═N—, —C═N—, —N═C— or —C(═N₂)—;
wherein yn is selected from the group consisting of integers ≥0, and yp is 0 or 1; wherein zn is selected from the group consisting of integers ≥0, and
0 or at least 1 methylene unit of Z is independently replaced by -Cyr-, —N(z^{x})C(O)—, —C(O)N(R^{zx})—, —C(O)—, —OC(O)—, —C(O)O—, —NR^{zx}—, —O—, —S—, —SO—, —SO₂—, —P(R^{zx})—, —P(═O)(R^{zx})—, —N(R^{x})SO₂—, —SO₂N(R^{zx})—, —C(═S)—, —C(═NR^{zx})—, —N═N—, —C═N—, —N═C— or —C(═N₂)—;
-Cyr- is selected from the group consisting of 6-10 membered arylene, 5-8 membered heteroarylene, 3-10 membered heterocyclylene and 3-10 membered saturated or partially unsaturated carbocyclylene, wherein -Cyr- is unsubstituted or independently substituted with at least 1 substituent R^{x};
wherein each R^{wa}, each R^{wb}, each R^{za}, each R^{zb}, each R^{wx}, each R^{cx} and each R^{cx} are each independently hydrogen, protium, deuterium, tritium, halogen, —NO₂, —CN, —OR, —SR, —N(R^{ra})(R^{rb}), —C(O)R, —C₂R^{r}, —C(O)C(O)R^{r}, —C(O)CH₂C(O)R^{r}, —S(O)R^{r}, —S(O)₂R^{r}, —C(O)N(R^{ra})(R^{rb}), —SO₂N(R^{ra})(R^{rb}), —OC(O)R^{r}, —N(R)SO₂R^{r}, or a C₁₋₆ aliphatic group optionally substituted with R;
wherein each R, each R^{ra} and each R^{rb} are each independently hydrogen, protium, deuterium, tritium, halogen, —NO2, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C1-6 aliphatic group;
-L_{b}- represents a peptide residue consisting of 2 to 7 amino acids;
-L_{c}- is selected from the group consisting of:

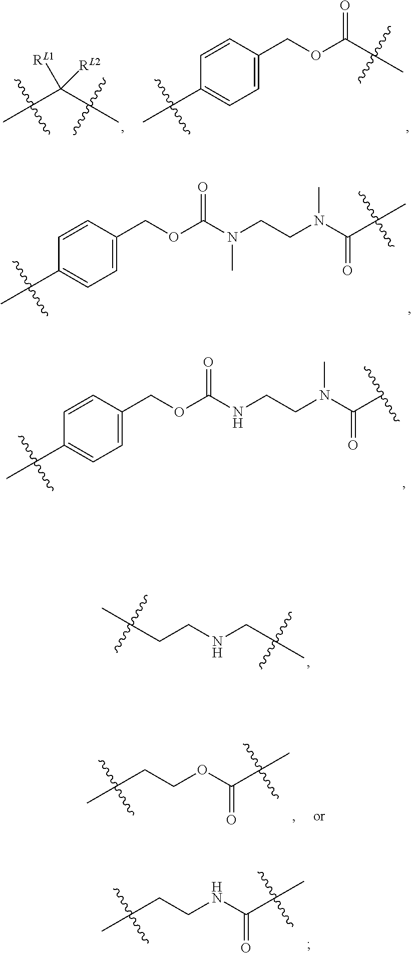

wherein R^{L1} and R^{L2} are each independently selected from the group consisting of hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H and a C1-6 aliphatic group.

65. The combination according to embodiment 64, binding agent for use according to embodiment 64, antibody-drug conjugate for use according to embodiment 64, or antibody-drug conjugate and binding agent for use according to embodiment 64, wherein the antibody-drug conjugate comprises a structure shown as formula (II-Dx):

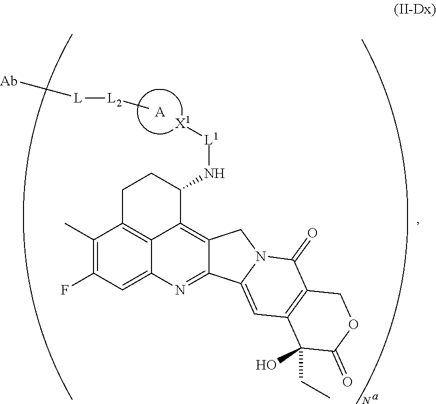

or a tautomer, mesomer, enantiomer thereof, or a pharmaceutically acceptable salt thereof wherein, Ab is an antibody or fragment thereof, and a connection number Na is an integer from 1 to 10.

66. The combination according to embodiment 64 or 65, binding agent for use according to embodiment 64 or 65, antibody-drug conjugate for use according to embodiment 64 or 65, or antibody-drug conjugate and binding agent for use according to embodiment 64 or 65, wherein -Lₐ- is selected from the group consisting of

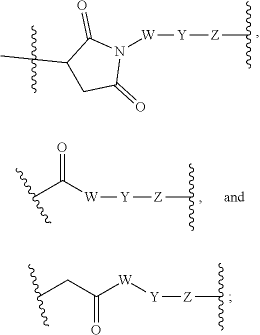

wherein W is —(C(R^{wa})(R^{wb}))_{wn}—, Y is —(OCH₂CH₂)_{yn}—O_{yp}—, and Z is —(C(R^{za})(R^{zb}))_{zn};
wherein wn is selected from the group consisting of integers from 2 to 6, and 0 or 1 methylene unit of W is independently replaced by -Cyr-, —N(R^{wx})C(O)—, —C(O)N(R^{wx})—, —C(O)—, —NR^{wx}— or —O—;
wherein yn is selected from the group consisting of integers from 0 to 12, and yp is 0 or 1;
wherein zn is selected from the group consisting of integers from 0 to 10, and 0 or 1 methylene unit of Z is independently replaced by -Cyr-, —N(R^{zx})C(O)—, —C(O)N(R^{zx})— or —C(O)—;
-Cyr- is selected from the group consisting of 6-10 membered arylene and 3-10 membered saturated or partially unsaturated carbocyclylene, wherein -Cyr- is unsubstituted or independently substituted with 1 to 3 substituent Rex;
wherein each R^{wa} each R^{wb}, each R^{za}, each R^{zb}, each R^{wx}, each R^{zx} and each R^{cx} are each independently hydrogen, protium, deuterium, tritium, halogen, —NO₂, —CN, —OR^{r}, —SR^{r}, —N(R^{ra})(R^{rb}), —C(O)R^{r}, —CO₂R^{r}, —C(O)C(O)R^{r}, —C(O)CH₂C(O)R^{r}, —S(O)R^{r}, —S(O)₂R^{r}, —C(O)N(R^{ra})(R^{rb}), —SO₂N(R^{ra})(R^{rb}), —OC(O)R^{r}, —N(R)SO₂R^{r}, or a C1.6 aliphatic group optionally substituted with R^{r};
wherein each R^{r}, each R^{a} and each R^{rb} are each independently hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO2H or a C1-6 aliphatic group;
-L_{b}- represents a peptide residue consisting of 2 to 7 amino acids, and the peptide residue of -L_{b}- is a peptide residue formed of amino acids selected from the group consisting of: phenylalanine, glycine, alanine, valine, citrulline, lysine, serine, glutamic acid and aspartic acid;
-L_{c}- is selected from the group consisting of

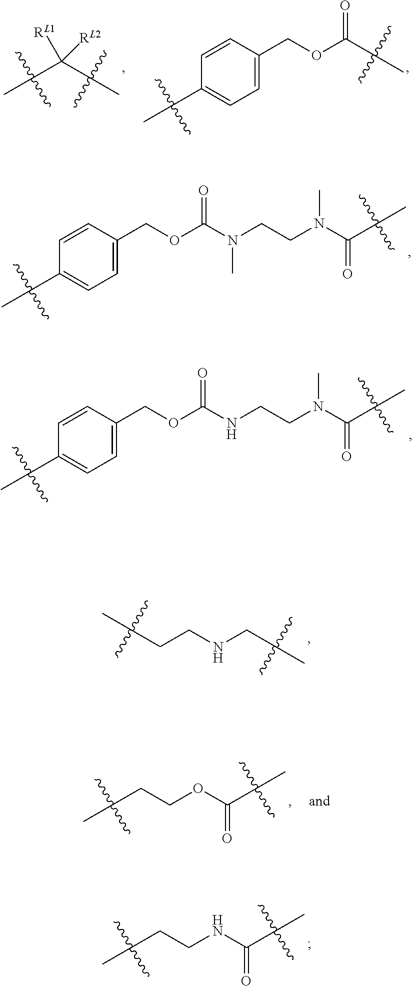

wherein R^{L1} and R^{L2} are each independently selected from the group consisting of hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H and a C₁₋₆ aliphatic group.

67. The combination according to any one of embodiments 64-66, binding agent for use according to any one of embodiments 64-66, antibody-drug conjugate for use according to any one of embodiments 64-66, or antibody-drug conjugate and binding agent for use according to any one of embodiments 64-66, wherein,
-Lₐ- is

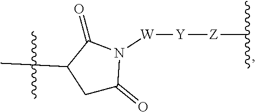

wherein W is —(C(R^{wa})(R^{wb}))_{wn}—, Y is —(OCH₂CH₂)_{yn}—O_{yp}—, and Z is —(C(R^{za})(R^{zb}))_{zn}; wherein wn is 1, 2, 3 or 6, and
1 methylene unit of W is independently replaced by -Cyr-, —N(R^{wx})C(O)—, —C(O)N(R^{wx})— or —C(O)—;
wherein yn is 0, 4 or 8, and yp is 0 or 1;
wherein zn is 1, 2 or 3, and
1 methylene unit of Z is independently replaced by -Cyr-, —N(R^{zx})C(O)—, —C(O)N(R^{zx})— or —C(O)—;
-Cyr- is 3-10 membered saturated carbocyclylene, wherein -Cyr- is unsubstituted or independently substituted with 1 to 3 substituent R^{cx};
wherein each R^{wa}, each R^{wb}, each R^{za}, each R^{zb}, each R^{wx}, each R^{zx} and each R^{cx} are each independently hydrogen, halogen, —OR^{r}, or a C₁₋₆ aliphatic group optionally substituted with R^{r};
wherein each R^{r} is independently hydrogen, halogen or a C₁₋₆ aliphatic group;
-L_{b}- represents a peptide residue consisting of 2 to 4 amino acids, and the peptide residue of -L_{b}- is a peptide residue formed of amino acids selected from the group consisting of: phenylalanine, glycine, alanine, valine, citrulline and lysine;
-L^{c}- is selected from the group consisting of:

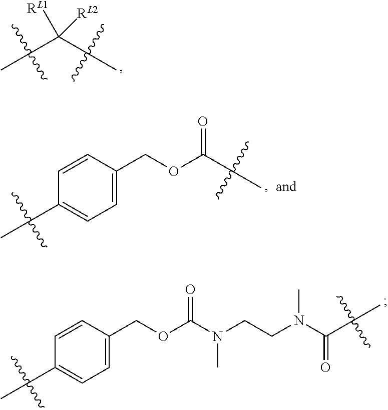

wherein R^{L1} and R^{L2} are each independently selected from the group consisting of hydrogen, halogen, —OH and a C₁₋₆ aliphatic group.

68. The combination according to any one of embodiments 64-67, binding agent for use according to any one of embodiments 64-67, antibody-drug conjugate for use according to any one of embodiments 64-67, or antibody-drug conjugate and binding agent for use according to any one of embodiments 64-67, wherein,
-Lₐ- is

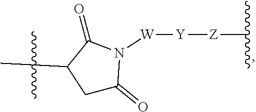

wherein W is —(C(R^{wa})(R^{wb}))_{wn}—, Y is —(OCH₂CH₂)_{yn}—O_{yp}—, and Z is —(C(R^{za})(R^{zb}))_{zn};
wherein wn is 1, 2, 3 or 6, and
1 methylene unit of W is independently replaced by -Cyr-, —N(R^{wx})C(O)—, —C(O)N(R^{wx})— or —C(O)—;
wherein yn is 0, 4 or 8, and yp is 0 or 1;
wherein zn is 1, 2 or 3, and
1 methylene unit of Z is independently replaced by -Cyr-, —N(R^{zx})C(O)—, —C(O)N(R^{zx})— or —C(O)—;
-Cyr- is 3-10 membered saturated carbocyclylene, wherein -Cyr- is unsubstituted or independently substituted with 1 to 3 substituent R^{cx};
wherein each R^{wa}, each R^{wb}, each R^{za}, each R^{zb}, each R^{wx}, each R^{zx} and each R^{cx} are each independently hydrogen, halogen, —OR^{r}, or a C₁₋₆ aliphatic group optionally substituted with R^{r};
wherein each R is independently hydrogen, halogen or a C₁₋₆ aliphatic group;
-L_{b}- is selected from the group consisting of

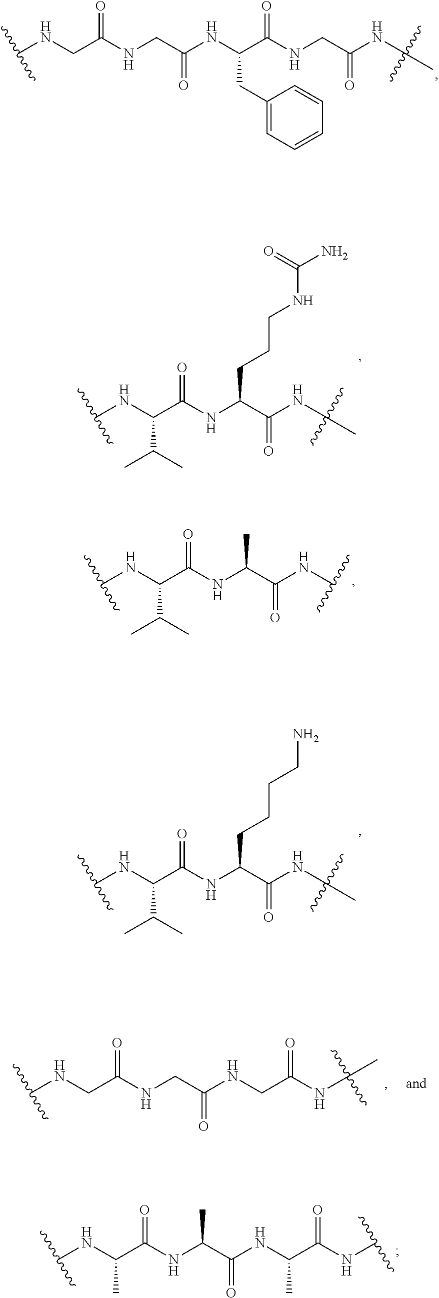 [SMILES: *NCC(=O)NCC(=O)N[C@@H](Cc1ccccc1)C(=O)NCC(=O)N* ; *NC(=O)[C@H](CCCNC(N)=O)NC(=O)[C@@H](N*)C(C)C ; *NC(=O)[C@H](C)NC(=O)[C@@H](N*)C(C)C ; *NC(=O)[C@H](CCCCN)NC(=O)[C@@H](N*)C(C)C ; *NCC(=O)NCC(=O)NCC(=O)N* ; *NC(=O)[C@H](C)NC(=O)[C@H](C)NC(=O)[C@H](C)N*]

### -L_{c}- is

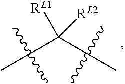

wherein R^{L1} and R^{L2} are each independently selected from the group consisting of hydrogen, halogen, —OH and a C₁₋₆ aliphatic group.

69. The combination according to any one of embodiments 64-68, binding agent for use according to any one of embodiments 64-68, antibody-drug conjugate for use according to any one of embodiments 64-68, or antibody-drug conjugate and binding agent for use according to any one of embodiments 64-68, wherein,
-Lₐ- is

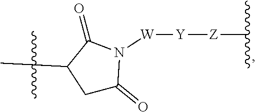

wherein W is —(C(R^{wa})(R^{wb}))_{wn}—, Y is —(OCH₂CH₂)_{yn}—O_{yp}—, and Z is —(C(R^{za})(R^{zb}))_{zn};
wherein wn is 1, 2, 3 or 6, and
1 methylene unit of W is independently replaced by -Cyr-, —N(R^{wx})C(O)—, —C(O)N(R^{wx})— or —C(O)—;
wherein yn is 0, 4 or 8, and yp is 0 or 1;
wherein zn is 1, 2 or 3, and
1 methylene unit of Z is independently replaced by -Cyr-, —N(R^{zx})C(O)—, —C(O)N(R^{zx})— or —C(O)—;
-Cyr- is 3-10 membered saturated carbocyclylene, wherein -Cyr- is unsubstituted or independently substituted with 1 to 3 substituent R^{cx};
wherein each R^{wa}, each R^{wb}, each R^{za}, each R^{zb}, each R^{wx}, each R^{zx} and each R^{cx} are each independently hydrogen, halogen, —OW, or a C₁₋₆ aliphatic group optionally substituted with R^{r};
wherein each R is independently hydrogen, halogen or a C₁₋₆ aliphatic group;
-L_{b}- is

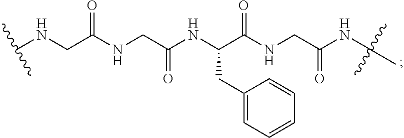 [SMILES: *NCC(=O)NCC(=O)N[C@@H](Cc1ccccc1)C(=O)NCC(=O)N*]

-L^{c}- is

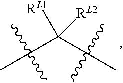

wherein R^{L1} and R^{L2} are each independently selected from the group consisting of hydrogen, halogen, —OH and a C1-6 aliphatic group.

70. The combination according to any one of embodiments 64-69, binding agent for use according to any one of embodiments 64-69, antibody-drug conjugate for use according to any one of embodiments 64-69, or antibody-drug conjugate and binding agent for use according to any one of embodiments 64-69, wherein,

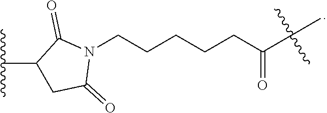 [SMILES: *C(=O)CCCCCN1C(=O)CC(*)C1=O]

71. The combination according to any one of embodiments 64-70, binding agent for use according to any one of embodiments 64-70, antibody-drug conjugate for use according to any one of embodiments 64-70, or antibody-drug conjugate and binding agent for use according to any one of embodiments 64-70, wherein,
-L_{b}- is:

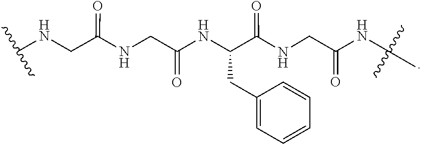 [SMILES: *NCC(=O)NCC(=O)N[C@@H](Cc1ccccc1)C(=O)NCC(=O)N*]

72. The combination according to any one of embodiments 64-71, binding agent for use according to any one of embodiments 64-71, antibody-drug conjugate for use according to any one of embodiments 64-71, or antibody-drug conjugate and binding agent for use according to any one of embodiments 64-71, wherein,
-L^{c}- is

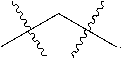 [SMILES: *C*]

73. The combination according to any one of embodiments 64-72, binding agent for use according to any one of embodiments 64-72, antibody-drug conjugate for use according to any one of embodiments 64-72, or antibody-drug conjugate and binding agent for use according to any one of embodiments 64-72, wherein,
-Lₐ-L_{b}-L_{c}- is

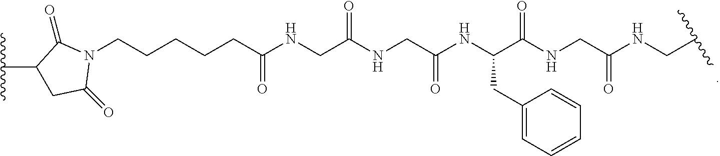 [SMILES: *CNC(=O)CNC(=O)[C@H](Cc1ccccc1)NC(=O)CNC(=O)CNC(=O)CCCCCN1C(=O)CC(*)C1=O]

74. The combination according to any one of embodiments 64-73, binding agent for use according to any one of embodiments 64-73, antibody-drug conjugate for use according to any one of embodiments 64-73, or antibody-drug conjugate and binding agent for use according to any one of embodiments 64-73, wherein ring A is selected from the group consisting of 3-10 membered saturated heterocyclyl and 3-10 membered saturated carbocyclyl.

75. The combination according to embodiment 74, binding agent for use according to embodiment 74, antibody-drug conjugate for use according to embodiment 74, or antibody-drug conjugate and binding agent for use according to embodiment 74, wherein ring A is 3-10 membered saturated carbocyclyl.

76. The combination according to embodiment 74, binding agent for use according to embodiment 74, antibody-drug conjugate for use according to embodiment 74, or antibody-drug conjugate and binding agent for use according to embodiment 74, wherein ring A is 3-6 membered saturated carbocyclyl, preferably wherein ring A is 4 membered saturated carbocyclyl.

77. The combination according to embodiment 74, binding agent for use according to embodiment 74, antibody-drug conjugate for use according to embodiment 74, or antibody-drug conjugate and binding agent for use according to embodiment 74, wherein ring A is 6 membered saturated carbocyclyl.

78. The combination according to embodiment 74, binding agent for use according to embodiment 74, antibody-drug conjugate for use according to embodiment 74, or antibody-drug conjugate and binding agent for use according to embodiment 74, wherein ring A is 3-10 membered saturated heterocyclyl, preferably wherein ring A is 3-6 membered saturated heterocyclyl.

79. The combination according to embodiment 74, binding agent for use according to a embodiment 74, antibody-drug conjugate for use according to embodiment 74, or antibody-drug conjugate and binding agent for use according to embodiment 74, wherein ring A is 3 membered saturated heterocyclyl.

80. The combination according to embodiment 78 or 79, binding agent for use according to embodiment 78 or 79, antibody-drug conjugate for use according to embodiment 78 or 79, or antibody-drug conjugate and binding agent for use according to embodiment 78 or 79, wherein ring A comprises 1 heteroatom, preferably wherein ring A comprises 1 nitrogen atom.

81. The combination according to any one of embodiments 63-80, binding agent for use according to any one of embodiments 63-80, antibody-drug conjugate for use according to any one of embodiments 63-80, or antibody-drug conjugate and binding agent for use according to any one of embodiments 63-80, wherein ring A is substituted with 0 substituent R^{1a}.

82. The combination according to embodiment 63, binding agent for use according to embodiment 63, antibody-drug conjugate for use according to embodiment 63, or antibody-drug conjugate and binding agent for use according to embodiment 63, wherein the payload-linker moiety of the antibody-drug conjugate has a structure shown as formula (II-Ax):

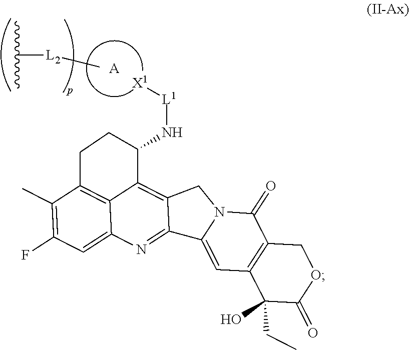

wherein, when ring A is 3-10 membered saturated or partially unsaturated carbocyclyl, ring A is substituted with p L², and L² is not Rn;
or, when ring A is 3-10 membered saturated or partially unsaturated heterocyclyl, ring A is substituted with p L².

83. The combination according to embodiment 82, binding agent for use according to embodiment 82, antibody-drug conjugate for use according to embodiment 82, or antibody-drug conjugate and binding agent for use according to embodiment 82, wherein ring A is substituted with 1 L².

84. The combination according to embodiment 82 or 83, binding agent for use according to embodiment 82 or 83, antibody-drug conjugate for use according to embodiment 82 or 83, or antibody-drug conjugate and binding agent for use according to embodiment 82 or 83, wherein m is 0, and L³ is a covalent bond.

85. The combination according to embodiment 82 or 83, binding agent for use according to embodiment 82 or 83, antibody-drug conjugate for use according to embodiment 82 or 83, or antibody-drug conjugate and binding agent for use according to embodiment 82 or 83, wherein m is 2, and L³ is —(C(R^{3a})(R^{3b}))₂—.

86. The combination according to any one of embodiments 82-85, binding agent for use according to any one of embodiments 82-85, antibody-drug conjugate for use according to any one of embodiments 82-85, or antibody-drug conjugate and binding agent for use according to any one of embodiments 82-85, wherein n is 0 or 1.

87. The combination according to embodiment 86, binding agent for use according to embodiment 86, antibody-drug conjugate for use according to embodiment 86, or antibody-drug conjugate and binding agent for use according to embodiment 86, wherein n is 0, and L¹ is a covalent bond.

88. The combination according to embodiment 86, binding agent for use according to embodiment 86, antibody-drug conjugate for use according to embodiment 86, or antibody-drug conjugate and binding agent for use according to embodiment 86, wherein n is 1, and L¹ is —C(R^{5a})(R^{5b})—.

89. The combination according to any one of embodiments 82-88, binding agent for use according to any one of embodiments 82-88, antibody-drug conjugate for use according to any one of embodiments 82-88, or antibody-drug conjugate and binding agent for use according to any one of embodiments 82-88, wherein 1 methylene unit of L¹ is replaced by —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—.

90. The combination according to embodiment 89, binding agent for use according to embodiment 89, antibody-drug conjugate for use according to embodiment 89, or antibody-drug conjugate and binding agent for use according to embodiment 89, wherein 1 methylene unit of L¹ is replaced by —C(O)—.

91. The combination according to any one of embodiments 82-90, binding agent for use according to any one of embodiments 82-90, antibody-drug conjugate for use according to any one of embodiments 82-90, or antibody-drug conjugate and binding agent for use according to any one of embodiments 82-90, wherein R² is selected from the group consistng of. —O—, —(R^{2a})N- and —S—.

92. The combination according to embodiment 91, binding agent for use according to embodiment 91, antibody-drug conjugate for use according to embodiment 91, or antibody-drug conjugate and binding agent for use according to embodiment 91, wherein R² is —O—.

93. The combination according to embodiment 91, binding agent for use according to embodiment 91, antibody-drug conjugate for use according to embodiment 91, or antibody-drug conjugate and binding agent for use according to embodiment 91, wherein R² is —(R^{2a})N—, preferably wherein R^{2a} is hydrogen.

94. The combination according to embodiment 91, binding agent for use according to embodiment 91, antibody-drug conjugate for use according to embodiment 91, or antibody-drug conjugate and binding agent for use according to embodiment 91, wherein R² is —HN—.

95. The combination according to any one of embodiments 82-94, binding agent for use according to any one of embodiments 82-94, antibody-drug conjugate for use according to any one of embodiments 82-94, or antibody-drug conjugate and binding agent for use according to any one of embodiments 82-94, wherein Ria is hydrogen.

96. The combination according to any one of embodiments 82-95, binding agent for use according to any one of embodiments 82-95, antibody-drug conjugate for use according to any one of embodiments 82-95, or antibody-drug conjugate and binding agent for use according to any one of embodiments 82-95, wherein R^{3a} and R^{3b} are each independently hydrogen.

97. The combination according to any one of embodiments 82-96, binding agent for use according to any one of embodiments 82-96, antibody-drug conjugate for use according to any one of embodiments 82-96, or antibody-drug conjugate and binding agent for use according to any one of embodiments 82-96, wherein R⁴ is hydrogen.

98. The combination according to any one of embodiments 82-97, binding agent for use according to any one of embodiments 82-97, antibody-drug conjugate for use according to any one of embodiments 82-97, or antibody-drug conjugate and binding agent for use according to any one of embodiments 82-97, wherein R⁶ is hydrogen.

99. The combination according to any one of embodiments 82-98, binding agent for use according to any one of embodiments 82-98, antibody-drug conjugate for use according to any one of embodiments 82-98, or antibody-drug conjugate and binding agent for use according to any one of embodiments 82-98, wherein R, R^{a} and R^{b} are each independently hydrogen.

100. The combination according to any one of embodiments 82-99, binding agent for use according to any one of embodiments 82-99, antibody-drug conjugate for use according to any one of embodiments 82-99, or antibody-drug conjugate and binding agent for use according to any one of embodiments 82-99, wherein the payload moiety of the antibody-drug conjugate comprises one of the following group of structures:

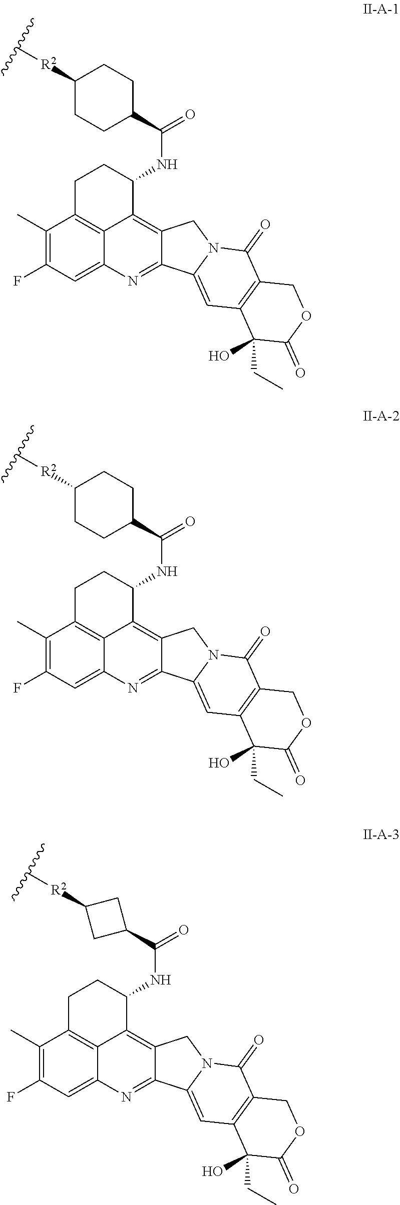

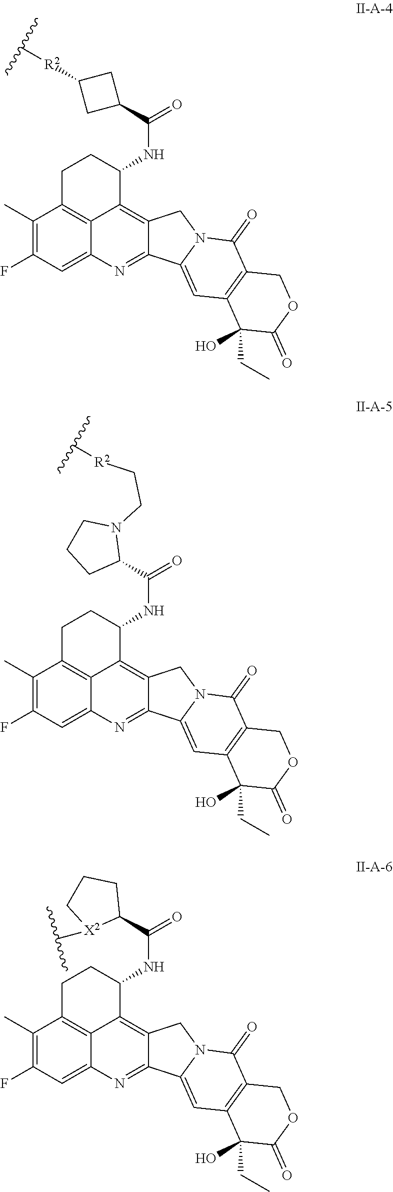

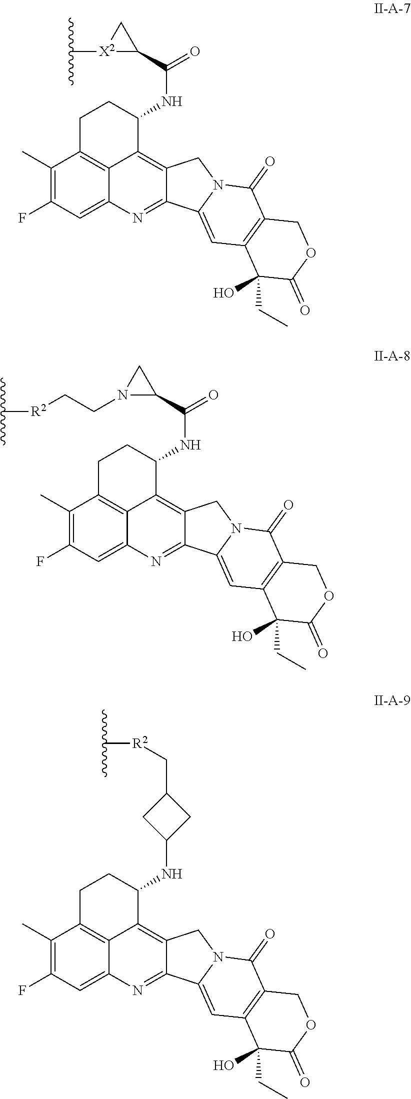

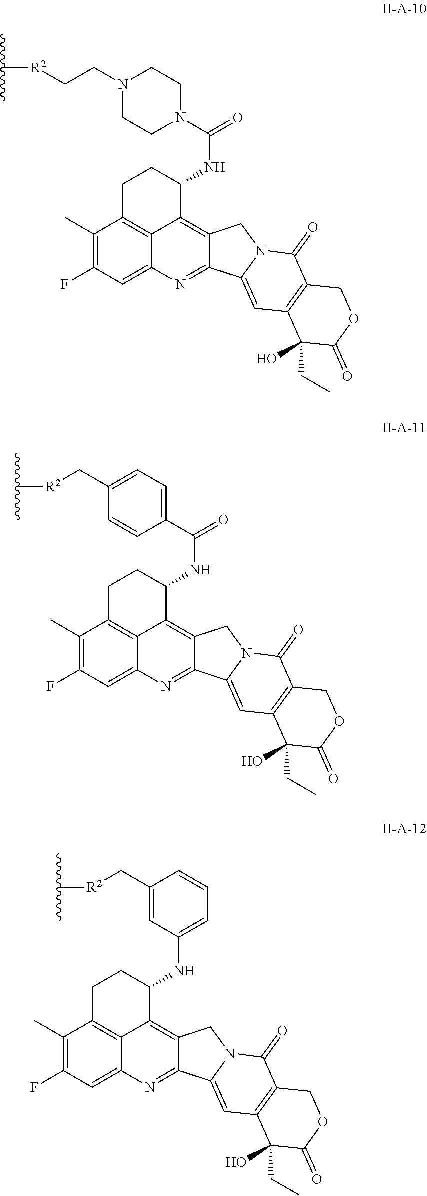

101. The combination according to embodiment 100, binding agent for use according to embodiment 100, antibody-drug conjugate for use according to embodiment 100, or antibody-drug conjugate and binding agent for use according to embodiment 100, wherein the payload moiety of the antibody-drug conjugate comprises the structure

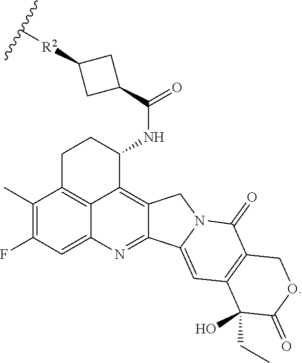

102. The combination according to embodiment 101, binding agent for use according to embodiment 101, antibody-drug conjugate for use according to embodiment 101, or antibody-drug conjugate and binding agent for use according to embodiment 101, wherein the payload of the linker-payload moiety of the antibody-drug conjugate has the structure

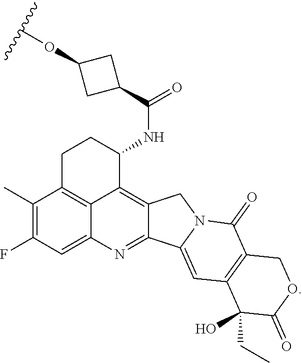 [SMILES: *O[C@H]1C[C@@H](C(=O)N[C@H]2CCc3c(C)c(F)cc4nc5c(c2c34)Cn2c-5cc3c(c2=O)COC(=O)[C@]3(O)CC)C1]

103. The combination according to embodiment 102, binding agent for use according to embodiment 102, antibody-drug conjugate for use according to embodiment 102, or antibody-drug conjugate and binding agent for use according to embodiment 102, wherein the linker-payload moiety of the antibody-drug conjugate has the structure

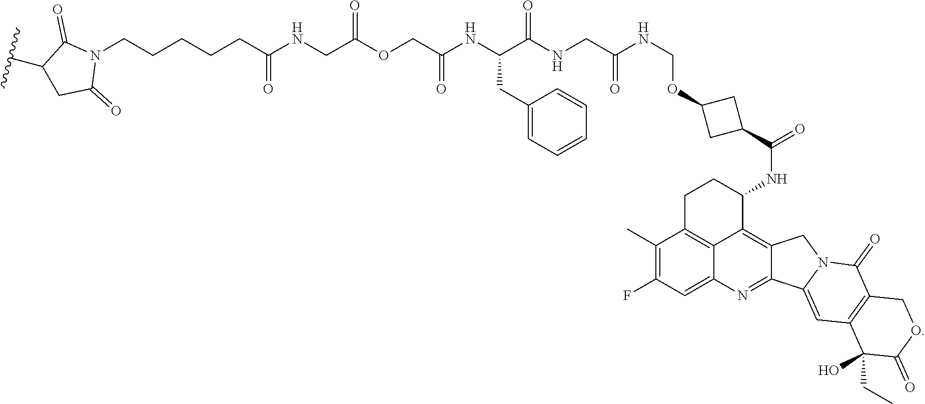 [SMILES: *C1CC(=O)N(CCCCCC(=O)NCC(=O)OCC(=O)N[C@@H](Cc2ccccc2)C(=O)NCC(=O)NCO[C@H]2C[C@@H](C(=O)N[C@H]3CCc4c(C)c(F)cc5nc6c(c3c45)Cn3c-6cc4c(c3=O)COC(=O)[C@]4(O)CC)C2)C1=O]

104. The combination according to any one of embodiments 1 or 5-40 or 54-95, binding agent for use according to any one of embodiments 2 or 5-40 or 54-95, antibody-drug conjugate for use according to any one of embodiments 3 or 5-40 or 54-95, or antibody-drug conjugate and binding agent for use according to any one of embodiments 4 or 5-40 or 54-95, wherein the target expressed on the surface of a tumour cell is HER2.

105. The combination according to any one of embodiments 1 or 5-44 or 63-104, binding agent for use according to any one of embodiments 2 or 5-44 or 63-104, antibody-drug conjugate for use according to any one of embodiments 3 or 5-44 or 63-104, or antibody-drug conjugate and binding agent for use according to any one of embodiments 4-44 or 63-104, the antibody-drug conjugate comprises an anti-HER2 antibody or fragment thereof.

106. The combination according to any one of embodiments 1 or 5-44 or 63-105, binding agent for use according to any one of embodiments 2 or 5-44 or 63-105, antibody-drug conjugate for use according to any one of embodiments 3 or 5-44 or 63-105, or antibody-drug conjugate and binding agent for use according to any one of embodiments 444 or 63-105, the antibody-drug conjugate comprises (a) trastuzumab or a fragment thereof; or (b) pertuzumab or a fragment thereof.

107. The combination according to any one of embodiments 104-106, binding agent for use according to any one of embodiments 104-106, antibody-drug conjugate for use according to any one of embodiments 104-106, or antibody-drug conjugate and binding agent for use according to any one of embodiments 104-106, wherein the antibody-drug conjugate comprises an anti-HER2 antibody or fragment thereof comprising:
(a) (i) a heavy chain variable region comprising: (i) a HCDR1 comprising or consisting of the amino acid sequence of SEQ ID NO: 49; (ii) a HCDR2 comprising or consisting of the amino acid sequence of SEQ ID NO: 50; and (iii) a HCDR3 comprising or consisting of the amino acid sequence of SEQ ID NO: 51; and a light chain variable region comprising: (i) a LCDR1 comprising or consisting of the amino acid sequence of SEQ ID NO: 52; (ii) a LCDR2 comprising or consisting of the amino acid sequence of SEQ ID NO: 53; and (iii) a LCDR3 comprising or consisting of the amino acid sequence of SEQ ID NO: 54, wherein the CDRs are defined according to the Kabat numbering system; and/or
(ii) a heavy chain variable region comprising: (i) a HCDR1 comprising or consisting of the amino acid sequence of SEQ ID NO: 55; (ii) a HCDR2 comprising or consisting of the amino acid sequence of SEQ ID NO: 56; and (iii) a HCDR3 comprising or consisting of the amino acid sequence of SEQ ID NO: 57; and a light chain variable region comprising: (i) a LCDR1 comprising or consisting of the amino acid sequence of SEQ ID NO: 58; (ii) a LCDR2 comprising or consisting of the amino acid sequence of SEQ ID NO: 59; and (iii) a LCDR3 comprising or consisting of the amino acid sequence of SEQ ID NO: 60, wherein the CDRs are defined according to the IMGT numbering system; or
(b) (i) a heavy chain variable region comprising: (i) a HCDR1 comprising or consisting of the amino acid sequence of SEQ ID NO: 65; (ii) a HCDR2 comprising or consisting of the amino acid sequence of SEQ ID NO: 66; and (iii) a HCDR3 comprising or consisting of the amino acid sequence of SEQ ID NO: 67; and a light chain variable region comprising: (i) a LCDR1 comprising or consisting of the amino acid sequence of SEQ ID NO: 68; (ii) a LCDR2 comprising or consisting of the amino acid sequence of SEQ ID NO: 69; and (iii) a LCDR3 comprising or consisting of the amino acid sequence of SEQ ID NO: 70, wherein the CDRs are defined according to the Kabat numbering system; and/or
(ii) a heavy chain variable region comprising: (i) a HCDR1 comprising or consisting of the amino acid sequence of SEQ ID NO: 71; (ii) a HCDR2 comprising or consisting of the amino acid sequence of SEQ ID NO: 72; and (iii) a HCDR3 comprising or consisting of the amino acid sequence of SEQ ID NO: 73; and a light chain variable region comprising: (i) a LCDR1 comprising or consisting of the amino acid sequence of SEQ ID NO: 74; (ii) a LCDR2 comprising or consisting of the amino acid sequence of SEQ ID NO: 75 and (iii) a LCDR3 comprising or consisting of the amino acid sequence of SEQ ID NO: 76, wherein the CDRs are defined according to the IMGT numbering system.

108. The combination according to any one of embodiments 104-107, binding agent for use according to any one of embodiments 104-107, antibody-drug conjugate for use according to any one of embodiments 104-107, or antibody-drug conjugate and binding agent for use according to any one of embodiments 104-107, wherein the antibody-drug conjugate comprises an anti-HER2 antibody or fragment thereof comprising:
(a) a heavy chain variable region comprising or consisting of the amino acid sequence of SEQ ID NO: 61 or an amino acid sequence having at least 80%, 85%, 90%, 95%, 96%, 97%, 98%, or 99% identity thereto; and a light chain variable region comprising or consisting of the amino acid sequence of SEQ ID NO: 62 or an amino acid sequence having at least 80%, 85%, 90%, 95%, 96%, 97%, 98%, or 99% identity thereto; or
(b) a heavy chain variable region comprising or consisting of the amino acid sequence of SEQ ID NO: 77 or an amino acid sequence having at least 80%, 85%, 90%, 95%, 96%, 97%, 98%, or 99% identity thereto; and a light chain variable region comprising or consisting of the amino acid sequence of SEQ ID NO: 78 or an amino acid sequence having at least 80%, 85%, 90%, 95%, 96%, 97%, 98%, or 99% identity thereto.

109. The combination according to any one of embodiments 104-108, binding agent for use according to any one of embodiments 104-108, antibody-drug conjugate for use according to any one of embodiments 104-108, or antibody-drug conjugate and binding agent for use according to any one of embodiments 104-108, wherein the antibody-drug conjugate comprises an anti-HER2 antibody or fragment thereof comprising:
(a) a heavy chain comprising or consisting of the amino acid sequence of SEQ ID NO: 63 or an amino acid sequence having at least 80%, 85%, 90%, 95%, 96%, 97%, 98%, or 99% identity thereto; and a light chain comprising or consisting of the amino acid sequence of SEQ ID NO: 64 or an amino acid sequence having at least 80%, 85%, 90%, 95%, 96%, 97%, 98%, or 99% identity thereto; or
(b) a heavy chain comprising or consisting of the amino acid sequence of SEQ ID NO: 79 or an amino acid sequence having at least 80%, 85%, 90%, 95%, 96%, 97%, 98%, or 99% identity thereto; and a light chain comprising or consisting of the amino acid sequence of SEQ ID NO: 80 or an amino acid sequence having at least 80%, 85%, 90%, 95%, 96%, 97%, 98%, or 99% identity thereto.

110. The combination according to any one of embodiments 104-109, binding agent for use according to any one of embodiments 104-109, antibody-drug conjugate for use according to any one of embodiments 104-109, or antibody-drug conjugate and binding agent for use according to any one of embodiments 104-109, wherein the antibody-drug conjugate has the structure (II-T):

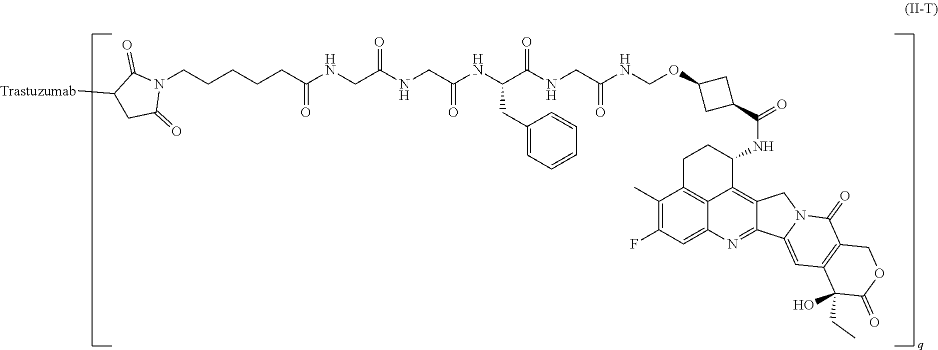

wherein,
q represents a connection number, and q is selected from the group consisting of integers from 1 to 16, preferably integers from 4 to 12, for example, q is an integer from 6 to 10, for example, q is an integer from 7 to 9, such as 7, 8 or 9, preferably 8; and
Trastuzumab is an anti-HER2 antibody comprising a heavy chain amino acid sequence as set forth in SEQ ID NO: 63 and a light chain amino acid sequence as set forth in SEQ ID NO: 64.

111. The combination according to any one of embodiments 1 or 5-110, binding agent for use according to any one of embodiments 2 or 5-110, antibody-drug conjugate for use according to any one of embodiments 3 or 5-110, or antibody-drug conjugate and binding agent for use according to any one of embodiments 4-110, wherein the first binding region which binds to PD-1, PD-L1, or both and which antagonizes the PD-1/PD-L1 interaction comprises an anti-PD-L1 antibody or a PD-L1 binding fragment thereof.

112. The combination according to any one of embodiments 1 or 5-111, binding agent for use according to any one of embodiments 2 or 5-111, antibody-drug conjugate for use according to any one of embodiments 3 or 5-111, or antibody-drug conjugate and binding agent for use according to any one of embodiments 4-111, wherein the first binding region which binds to PD-1, PD-L1, or both and which antagonizes the PD-1/PD-L1 interaction comprises:
(i-3a) an antigen-binding fragment from a camelid-derived heavy chain antibody (VHH) comprising the HCDR1, HCDR2, and HCDR3 sequences set forth in: SEQ ID NO: 120, 121, and 122, respectively;
(i-3b) an antigen-binding fragment from a camelid-derived heavy chain antibody (VHH) comprising the HCDR1, HCDR2, and HCDR3 sequences set forth in: SEQ ID NO: 123, 124, and 125, respectively; and/or
(i-3c) the amino acid sequence of SEQ ID NO: 126 or an amino acid sequence having at least 80%, at least 85%, at least 90%, at least 95%, at least 96%, at least 97%, at least 98%, at least 99%, or 100% sequence identity to the amino acid sequence SEQ ID NO: 126, wherein the amino acid sequence having at least 80%, at least 85%, at least 90%, at least 95%, at least 96%, at least 97%, at least 98%, at least 99%, or 100% sequence identity to the amino acid sequence SEQ ID NO: 126 preferably comprises the HCDR1, HCDR2, and HCDR3 sequences set forth in: SEQ ID NO: 120, 121, and 122, respectively, or comprises the HCDR1, HCDR2, and HCDR3 sequences set forth in: SEQ ID NO: 123, 124, and 125, respectively.

113. The combination according to any one of embodiments 1 or 5-112, binding agent for use according to any one of embodiments 2 or 5-112, antibody-drug conjugate for use according to any one of embodiments 3 or 5-112, or antibody-drug conjugate and binding agent for use according to any one of embodiments 4-112, wherein the first binding region which binds to PD-1, PD-L1, or both and which antagonizes the PD-1/PD-L1 interaction comprises a single-chain fragment (e.g., scFv) or a VHH.

114. The combination according to any one of embodiments 1 or 5-113, binding agent for use according to any one of embodiments 2 or 5-113, antibody-drug conjugate for use according to any one of embodiments 3 or 5-113, or antibody-drug conjugate and binding agent for use according to any one of embodiments 4-113, wherein the first binding region which binds to PD-1, PD-L1, or both and which antagonizes the PD-1/PD-L1 interaction comprises:
(i-4a) a heavy chain variable region (VH) comprising the HCDR1, HCDR2, and HCDR3 sequences of SEQ ID NO: 133, and/or a light chain variable region (VL) comprising the LCDR1, LCDR2, and LCDR3 sequences of SEQ ID NO: 134;
(i-4b) a heavy chain variable region (VH) comprising the HCDR1, HCDR2, and HCDR3 sequences set forth in: SEQ ID NO: 127, 128, and 129, respectively, and/or a light chain variable region (VL) comprising the LCDR1, LCDR2, and LCDR3 sequences set forth in: SEQ ID NO: 130, 131, and 132, respectively;
(i-4c) a heavy chain variable region (VH) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 133, and a light chain variable region (VL) comprising an amino acid sequence having at least 80%, at least 85%, at least 90%, at least 95%, at least 96%, at least 97%, at least 98%, at least 99%, or 100% sequence identity to the amino acid sequence SEQ ID NO: 134; or
(i-4d) a heavy chain variable region (VH) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 133, and a light chain variable region (VL) comprising an amino acid sequence having at least 80%, at least 85%, at least 90%, at least 95%, at least 96%, at least 97%, at least 98%, at least 99%, or 100% sequence identity to the amino acid sequence SEQ ID NO: 134, wherein the VH comprises the HCDR1, HCDR2, and HCDR3 sequences set forth in: SEQ ID NO: 127, 128, and 129, respectively, and the VL comprises the LCDR1, LCDR2, and LCDR3 sequences set forth in: SEQ ID NO: 130, 131, and 132, respectively

115. The combination according to embodiment 111, binding agent for use according to embodiment 111, antibody-drug conjugate for use according to embodiment 11, or antibody-drug conjugate and binding agent for use according to embodiment 111, wherein the anti-PD-L1 antibody is selected from the group consisting of atezolizumab, durvalumab, avelumab, and PD-L1 binding fragments thereof.

116. The combination according to any one of embodiments 1 or 5-110, binding agent for use according to any one of embodiments 2 or 5-110, antibody-drug conjugate for use according to any one of embodiments 3 or 5-115, or antibody-drug conjugate and binding agent for use according to any one of embodiments 4 or 5-110, wherein the first binding region which binds to PD-1, PD-L1, or both and which antagonizes the PD-1/PD-L1 interaction comprises an anti-PD-1 antibody or a PD-1 binding fragment thereof.

117. The combination according to any one of embodiments 1 or 5-110 or 116, binding agent for use according to any one of embodiments 2 or 5-110 or 116, antibody-drug conjugate for use according to any one of embodiments 3 or 5-110 or 116, or antibody-drug conjugate and binding agent for use according to any of embodiments 4-110 or 116, wherein the first binding region which binds to PD-1, PD-L1, or both and which antagonizes the PD-1/PD-L1 interaction comprises:
(i-1a) a heavy chain variable region (VH) comprising the HCDR1, HCDR2, and HCDR3 sequences set forth in: SEQ ID NO: 178, 179, and 180, respectively, and/or a light chain variable region (VL) comprising the LCDR1, LCDR2, and LCDR3 sequences set forth in: SEQ ID NO: 181, 182, and 183, respectively;
(i-1b) a heavy chain variable region (VH) comprising the HCDR1, HCDR2, and HCDR3 sequences set forth in: SEQ ID NO: 184, 185, and 186, respectively, and/or a light chain variable region (VL) comprising the LCDR1, LCDR2, and LCDR3 sequences set forth in: SEQ ID NO: 187, 188, and 183, respectively;
(i-1c) the amino acid sequence of SEQ ID NO: 189 or an amino acid sequence having at least 80%, at least 85%, at least 90%, at least 95%, at least 96%, at least 97%, at least 98%, at least 99%, or 100% sequence identity to the amino acid sequence SEQ ID NO: 189;
(i-1d) an amino acid sequence having at least 80%, at least 85%, at least 90%, at least 95%, at least 96%, at least 97%, at least 98%, at least 99%, or 100% sequence identity to the amino acid sequence SEQ ID NO: 189, and comprising the HCDR1, HCDR2, HCDR3, LCDR1, LCDR2, and LCDR3 sequences set forth in: SEQ ID NO: 178, 179, 180, 181, 182, and 183, respectively; or
(i-1e) an amino acid sequence having at least 80%, at least 85%, at least 90%, at least 95%, at least 96%, at least 97%, at least 98%, at least 99%, or 100% sequence identity to the amino acid sequence SEQ ID NO: 189, and comprising the HCDR1, HCDR2, HCDR3, LCDR1, LCDR2, and LCDR3 sequences set forth in: SEQ ID NO: 184, 185, 186, 187, 188, and 183, respectively.

118. The combination according to any of embodiments 1 or 5-110 or 116, binding agent for use according to any of embodiments 2 or 5-110 or 116, antibody-drug conjugate for use according to any of embodiments 3 or 5-110 or 116, or antibody-drug conjugate and binding agent for use according to any of embodiments 4-110 or 116, wherein the first binding region which binds to PD-1, PD-L1, or both and which antagonizes the PD-1/PD-L1 interaction comprises:
(i-2a) an antigen-binding fragment from a camelid-derived heavy chain antibody (VHH) comprising the HCDR1, HCDR2, and HCDR3 sequences set forth in: SEQ ID NO: 110, 111, and 112, respectively, or a VHH comprising the HCDR1, HCDR2, and HCDR3 sequences set forth in: SEQ ID NO: 110, 111, and 113, respectively;
(i-2b) an antigen-binding fragment from a camelid-derived heavy chain antibody (VHH) comprising the HCDR1, HCDR2, and HCDR3 sequences set forth in: SEQ ID NO: 114, 115, and 116, respectively, or a VHH comprising the HCDR1, HCDR2, and HCDR3 sequences set forth in: SEQ ID NO: 114, 115, and 117, respectively;
(i-2c) the amino acid sequence of SEQ ID NO: 118 or an amino acid sequence having at least 80%, at least 85%, at least 90%, at least 95%, at least 96%, at least 97%, at least 98%, at least 99%, or 100% sequence identity to the amino acid sequence SEQ ID NO: 118, wherein the amino acid sequence having at least 80%, at least 85%, at least 90%, at least 95%, at least 96%, at least 97%, at least 98%, at least 99%, or 100% sequence identity to the amino acid sequence SEQ ID NO: 118 preferably comprises the HCDR1, HCDR2, and HCDR3 sequences set forth in: SEQ ID NO: 110, 111, and 112, respectively, or comprises the HCDR1, HCDR2, and HCDR3 sequences set forth in: SEQ ID NO: 114, 115, and 116, respectively; or
(i-2d) the amino acid sequence of SEQ ID NO: 119 or an amino acid sequence having at least 80%, at least 85%, at least 90%, at least 95%, at least 96%, at least 97%, at least 98%, at least 99%, or 100% sequence identity to the amino acid sequence SEQ ID NO: 119, wherein the amino acid sequence having at least 80%, at least 85%, at least 90%, at least 95%, at least 96%, at least 97%, at least 98%, at least 99%, or 100% sequence identity to the amino acid sequence SEQ ID NO: 119 preferably comprises the HCDR1, HCDR2, and HCDR3 sequences set forth in: SEQ ID NO: 110, 111, and 113, respectively, or comprises the HCDR1, HCDR2, and HCDR3 sequences set forth in: SEQ ID NO: 114, 115, and 117, respectively.

119. The combination according to embodiment 116, binding agent for use according to embodiment 116, antibody-drug conjugate for use according to embodiment 116, or antibody-drug conjugate and binding agent for use according to embodiment 116, wherein the anti-PD-1 antibody is selected from the group consisting of pembrolizumab, nivolumab, cemiplimab, dostarlimab, toripalimab, retifanlimab, and PD-1 binding fragments thereof.

120. The combination according to any one of embodiments 1 or 5-119, binding agent for use according to any one of embodiments 2 or 5-119, antibody-drug conjugate for use according to any one of embodiments 3 or 5-119, or antibody-drug conjugate and binding agent for use according to any one of embodiments 4-119, wherein the second binding region which binds to VEGF, VEGF receptor (VEGFR), or both and which antagonizes the VEGF/VEGFR interaction comprises an anti-VEGF antibody (preferably an anti-VEGF-A antibody) or a VEGF binding fragment thereof.

121. The combination according to any one of embodiments 1 or 5-120, binding agent for use according to any one of embodiments 2 or 5-120, antibody-drug conjugate for use according to any one of embodiments 3 or 5-120, or antibody-drug conjugate and binding agent for use according to one of embodiments 4-120, wherein the second binding region which binds to VEGF, VEGF receptor (VEGFR), or both and which antagonizes the VEGF/VEGFR interaction comprises:
(ii-1a) a heavy chain variable region (VH) comprising the HCDR1, HCDR2, and HCDR3 sequences of SEQ ID NO: 146, and a light chain variable region (VL) comprising the LCDR1, LCDR2, and LCDR3 sequences of SEQ ID NO: 147;
(ii-1b) a heavy chain variable region (VH) comprising the HCDR1, HCDR2, and HCDR3 sequences set forth in: SEQ ID NO: 135, 136, and 137, respectively, and a light chain variable region (VL) comprising the LCDR1, LCDR2, and LCDR3 sequences set forth in: SEQ ID NO: 138, 139, and 140, respectively;
(ii-1c) a heavy chain variable region (VH) comprising the HCDR1, HCDR2, and HCDR3 sequences set forth in: SEQ ID NO: 141, 142, and 143, respectively, and a light chain variable region (VL) comprising the LCDR1, LCDR2, and LCDR3 sequences set forth in: SEQ ID NO: 144, 145, and 140, respectively;
(ii-1d) a heavy chain variable region (VH) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 146, and a light chain variable region (VL) comprising an amino acid sequence having at least 80%, at least 85%, at least 90%, at least 95%, at least 96%, at least 97%, at least 98%, at least 99%, or 100% sequence identity to the amino acid sequence SEQ ID NO: 147;
(ii-1e) a heavy chain variable region (VH) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 146, and a light chain variable region (VL) comprising an amino acid sequence having at least 80%, at least 85%, at least 90%, at least 95%, at least 96%, at least 97%, at least 98%, at least 99%, or 100% sequence identity to the amino acid sequence SEQ ID NO: 147, wherein the VH comprises the HCDR1, HCDR2, and HCDR3 sequences set forth in: SEQ ID NO: 135, 136, and 137, respectively, and the VL comprises the LCDR1, LCDR2, and LCDR3 sequences set forth in: SEQ ID NO: 138, 139, and 140, respectively;
(ii-1f) a heavy chain variable region (VH) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 146, and a light chain variable region (VL) comprising an amino acid sequence having at least 80%, at least 85%, at least 90%, at least 95%, at least 96%, at least 97%, at least 98%, at least 99%, or 100% sequence identity to the amino acid sequence SEQ ID NO: 147, wherein the VH comprises the HCDR1, HCDR2, and HCDR3 sequences set forth in: SEQ ID NO: 141, 142, and 143, respectively, and the VL comprises the LCDR1, LCDR2, and LCDR3 sequences set forth in: SEQ ID NO: 144, 145, and 140, respectively;
(ii-1g) a heavy chain (HC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 151 and a light chain (LC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 152;
(ii-1h) a heavy chain (HC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 151 and a light chain (LC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 152, wherein the HC comprises the HCDR1, HCDR2, and HCDR3 sequences set forth in: SEQ ID NO: 135, 136, and 137, respectively, and the LC comprises the LCDR1, LCDR2, and LCDR3 sequences set forth in: SEQ ID NO: 138, 139, and 140, respectively; or
(ii-1i) a heavy chain (HC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 151 and a light chain (LC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 152, wherein the HC comprises the HCDR1, HCDR2, and HCDR3 sequences set forth in: SEQ ID NO: 141, 142, and 143, respectively, and the LC comprises the LCDR1, LCDR2, and LCDR3 sequences set forth in: SEQ ID NO: 144, 145, and 140, respectively.

122. The combination according to any one of embodiments 1 or 5-121, binding agent for use according to any one of embodiments 2 or 5-121, antibody-drug conjugate for use according to any one of embodiments 3 or 5-121, or antibody-drug conjugate and binding agent for use according to any one of embodiments 4-121, wherein the second binding region which binds to VEGF, VEGF receptor (VEGFR), or both and which antagonizes the VEGF/VEGFR interaction is in the format of a full-length antibody or a VEGF binding antibody fragment thereof.

123. The combination according to embodiment 120 or 122, binding agent for use according to embodiment 120 or 122, antibody-drug conjugate for use according to embodiment 120 or 122, or antibody-drug conjugate and binding agent for use according to embodiment 120 or 122, wherein the anti-VEGF antibody is selected from bevacizumab, ranibizumab, and VEGF binding fragments thereof.

124. The combination according to any one of embodiments 1 or 5-119, binding agent for use according to any one of embodiments 2 or 5-119, antibody-drug conjugate for use according to any one of embodiments 3 or 5-119, or antibody-drug conjugate and binding agent for use according to any of embodiments 4 or 5-119, wherein the second binding region which binds to VEGF, VEGF receptor (VEGFR), or both and which antagonizes the VEGF/VEGFR interaction comprises a VEGFR domain, such as a VEGFR-1 domain comprising the amino acid sequence of SEQ ID NO: 153, a VEGFR-2 domain comprising the amino acid sequence of SEQ ID NO: 154, a VEGFR-3 domain comprising the amino acid sequence of SEQ ID NO: 155, or a combination thereof.

125. The combination according to any one of embodiments 1 or 5-119 or 124, binding agent for use according to any one of embodiments 2 or 5-119 or 124, antibody-drug conjugate for use according to any one of embodiments 3 or 5-119 or 124, or antibody-drug conjugate and binding agent for use according to any of embodiments 4-119 or 124, wherein the second binding region which binds to VEGF, VEGF receptor (VEGFR), or both and which antagonizes the VEGF/VEGFR interaction comprises at least one VEGFR-1 domain, or at least one VEGFR-2 domain, or at least one VEGFR-3 domain, or at least one VEGFR-1 domain and at least one VEGFR-2 domain, such as two VEGFR-1 domains, or two VEGFR-2 domains, or two VEGFR-1 domains and two VEGFR-2 domains, or two VEGFR-3 domains.

126. The combination according to any of embodiments 1, 5-119, 124 or 125 117-118, binding agent for use according to any of embodiments 2, 5-119, 124 or 125, antibody-drug conjugate for use according to any of embodiments 3, 5-119, 124 or 125, or antibody-drug conjugate and binding agent for use according to any of embodiments 4-119, 124 or 125, wherein the second binding region which binds to VEGF, VEGF receptor (VEGFR), or both and which antagonizes the VEGF/VEGFR interaction comprises:
(ii-2a) the amino acid sequence of SEQ ID NO: 153 or an amino acid sequence having at least 80%, at least 85%, at least 90%, at least 95%, at least 96%, at least 97%, at least 98%, at least 99%, or 100% sequence identity to the amino acid sequence of SEQ ID NO: 153;
(ii-2b) the amino acid sequence of SEQ ID NO: 154 or an amino acid sequence having at least 80%, at least 85%, at least 90%, at least 95%, at least 96%, at least 97%, at least 98%, at least 99%, or 100% sequence identity to the amino acid sequence of SE Q ID NO: 154;
(ii-2c) the amino acid sequence of SEQ ID NO: 155 or an amino acid sequence having at least 80%, at least 85%, at least 90%, at least 95%, at least 96%, at least 97%, at least 98%, at least 99%, or 100% sequence identity to the amino acid sequence of SEQ ID NO: 155;
(ii-2d) the amino acid sequence of SEQ ID NO: 156 or an amino acid sequence having at least 80%, at least 85%, at least 90%, at least 95%, at least 96%, at least 97%, at least 98%, at least 99%, or 100% sequence identity to the amino acid sequence of SEQ ID NO: 156; or
(ii-2e) the amino acid sequence of SEQ ID NO: 157 or an amino acid sequence having at least 80%, at least 85%, at least 90%, at least 95%, at least 96%, at least 97%, at least 98%, at least 99%, or 100% sequence identity to the amino acid sequence of SEQ ID NO: 157.

127. The combination according to any one of embodiments 1 or 5-126, binding agent for use according to any one of embodiments 2 or 5-126, antibody-drug conjugate for use according to any one of embodiments 3 or 5-126, or antibody-drug conjugate and binding agent for use according to any one of embodiments 4-126, wherein the binding agent is a multispecific antibody, such as a bispecific antibody.

128. The combination according to embodiment 127, binding agent for use according to embodiment 127, antibody-drug conjugate for use according to embodiment 127, or antibody-drug conjugate and binding agent for use according to embodiment 127, wherein the first binding region of the multispecific antibody binds to PD-L1 and the second binding region binds to VEGF, preferably to VEGF-A.

129. The combination according to embodiment 128, binding agent for use according to embodiment 128, antibody-drug conjugate for use according to embodiment 128, or antibody-drug conjugate and binding agent for use according to embodiment 128, wherein the multispecific antibody comprises an anti-VEGF antibody and at least one anti-PD-L1 single domain antibody, preferably an anti-VEGF antibody and two anti-PD-L1 single domain antibodies, more preferably an anti-VEGF antibody and two anti-PD-L1 VHHs.

130. The combination according to embodiment 129, binding agent for use according to embodiment 129, antibody-drug conjugate for use according to embodiment 129, or antibody-drug conjugate and binding agent for use according to embodiment 129, wherein each heavy chain of the anti-VEGF antibody is terminally fused to an anti-PD-L1 single domain antibody, preferably via a linker.

131. The combination according to any of embodiments 128-130, binding agent for use according to any one of embodiments 128-130, antibody-drug conjugate for use according to any one of embodiments 128-130, or antibody-drug conjugate and binding agent for use according to any one of embodiments 128-130, wherein the binding agent comprises: (A) (1) at least one anti-PD-L1 single domain antibody comprising the HCDR1, HCDR2, and HCDR3 sequences set forth in: SEQ ID NO: 120, 121, and 122, respectively, or comprising the HCDR1, HCDR2, and HCDR3 sequences set forth in: SEQ ID NO: 123, 124, and 125, respectively; and/or (2) an anti-VEGF antibody comprising (i) a heavy chain variable region (VH) comprising the HCDR1, HCDR2, and HCDR3 sequences set forth in: SEQ ID NO: 135, 136, and 137, respectively, and a light chain variable region (VL) comprising the LCDR1, LCDR2, and LCDR3 sequences set forth in: SEQ ID NO: 138, 139, and 140, respectively; or (ii) a VH comprising the HCDR1, HCDR2, and HCDR3 sequences set forth in: SEQ ID NO: 141, 142, and 143, respectively, and a VL comprising the LCDR1, LCDR2, and LCDR3 sequences set forth in: SEQ ID NO: 144, 145, and 140, respectively; or
(B) (1) at least one anti-PD-L1 single domain antibody comprising the amino acid sequence of SEQ ID NO: 126 or an amino acid sequence having at least 80%, at least 85%, at least 90%, at least 95%, at least 96%, at least 97%, at least 98%, at least 99%, or 100% sequence identity to the amino acid sequence SEQ ID NO: 126, wherein the amino acid sequence having at least 80%, at least 85%, at least 90%, at least 95%, at least 96%, at least 97%, at least 98%, at least 99%, or 100% sequence identity to the amino acid sequence SEQ ID NO: 126 preferably comprises the HCDR1, HCDR2, and HCDR3 sequences set forth in: SEQ ID NO: 120, 121, and 122, respectively, or the HCDR1, HCDR2, and HCDR3 sequences set forth in: 123, 124, and 125, respectively; and/or (2) an anti-VEGF antibody comprising (i) a heavy chain variable region (VH) comprising the HCDR1, HCDR2, and HCDR3 sequences set forth in: SEQ ID NO: 135, 136, and 137, respectively, and a light chain variable region (VL) comprising the LCDR1, LCDR2, and LCDR3 sequences set forth in: SEQ ID NO: 138, 139, and 140, respectively; or (ii) a VH comprising the HCDR1, HCDR2, and HCDR3 sequences set forth in: SEQ ID NO: 141, 142, and 143, respectively, and a VL comprising the LCDR1, LCDR2, and LCDR3 sequences set forth in: SEQ ID NO: 144, 145, and 140, respectively.

132. The combination according to any one of embodiments 128-131, binding agent for use according to any one of embodiments 128-131, antibody-drug conjugate for use according to any one of embodiments 128-131, or antibody-drug conjugate and binding agent for use according to any one of embodiments 128-131, wherein the anti-VEGF antibody comprises:
(ii-1d) a heavy chain variable region (VH) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 146, and a light chain variable region (VL) comprising an amino acid sequence having at least 80%, at least 85%, at least 90%, at least 95%, at least 96%, at least 97%, at least 98%, at least 99%, or 100% sequence identity to the amino acid sequence SEQ ID NO: 147;
(ii-1e) a heavy chain variable region (VH) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 146, and a light chain variable region (VL) comprising an amino acid sequence having at least 80%, at least 85%, at least 90%, at least 95%, at least 96%, at least 97%, at least 98%, at least 99%, or 100% sequence identity to the amino acid sequence SEQ ID NO: 147, wherein the VH comprises the HCDR1, HCDR2, and HCDR3 sequences set forth in: SEQ ID NO: 135, 136, and 137, respectively, and the VL comprises the LCDR1, LCDR2, and LCDR3 sequences set forth in: SEQ ID NO: 138, 139, and 140, respectively;
(ii-1f) a heavy chain variable region (VH) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 146, and a light chain variable region (VL) comprising an amino acid sequence having at least 80%, at least 85%, at least 90%, at least 95%, at least 96%, at least 97%, at least 98%, at least 99%, or 100% sequence identity to the amino acid sequence SEQ ID NO: 147, wherein the VH comprises the HCDR1, HCDR2, and HCDR3 sequences set forth in: SEQ ID NO: 141, 142, and 143, respectively, and the VL comprises the LCDR1, LCDR2, and LCDR3 sequences set forth in: SEQ ID NO: 144, 145, and 140, respectively;
(ii-1g) a heavy chain (HC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 151, and a light chain (LC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 152;
(ii-1h) a heavy chain (HC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 151, and a light chain (LC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 152, wherein the HC comprises the HCDR1, HCDR2, and HCDR3 sequences set forth in: SEQ ID NO: 135, 136, and 137, respectively, and the LC comprises the LCDR1, LCDR2, and LCDR3 sequences set forth in: SEQ ID NO: 138, 139, and 140, respectively; or
(ii-1i) a heavy chain (HC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 151, and a light chain (LC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 152, wherein the HC comprises the HCDR1, HCDR2, and HCDR3 sequences set forth in: SEQ ID NO: 141, 142, and 143, respectively, and the LC comprises the LCDR1, LCDR2, and LCDR3 sequences set forth in: SEQ ID NO: 144, 145, and 140, respectively.

133. The combination according to any one of embodiments 128-132, binding agent for use according to any one of embodiments 128-132, antibody-drug conjugate for use according to any one of embodiments 128-132, or antibody-drug conjugate and binding agent for use according to any one of embodiments 128-132, wherein the binding agent comprises (A) a heavy chain (HC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 158, and (B) a light chain (LC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 152, wherein (I) the HC comprises (i) the HCDR1, HCDR2, and HCDR3 sequences set forth in: SEQ ID NO: 135, 136, and 137, respectively, and (ii) the HCDR1, HCDR2, and HCDR3 sequences set forth in: SEQ ID NO: 120, 121, and 122, respectively, or the HC comprises (i′) the HCDR1, HCDR2, and HCDR3 sequences set forth in: SEQ ID NO: 141, 142, and 143, respectively, and (ii′) the HCDR1, HCDR2, and HCDR3 sequences set forth in: SEQ ID NO: 123, 124, and 125, respectively, and (II) the LC comprises the LCDR1, LCDR2, and LCDR3 sequences set forth in: SEQ ID NO: 138, 139, and 140, respectively, or the LCDR1, LCDR2, and LCDR3 sequences set forth in: SEQ ID NO: 144, 145, and 140, respectively.

134. The combination according to any one of embodiments 128-133, binding agent for use according to any one of embodiments 128-133, antibody-drug conjugate for use according to any one of embodiments 128-133, or antibody-drug conjugate and binding agent for use according to any one of embodiments 128-133, wherein the binding agent comprises (A) two heavy chains (HCs) each comprising the amino acid sequence of SEQ ID NO: 158, and (B) two light chains (LCs) each comprising the amino acid sequence of SEQ ID NO: 152.

135. The combination according to embodiment 127, binding agent for use according to embodiment 127, antibody-drug conjugate for use according to embodiment 127, or antibody-drug conjugate and binding agent for use according to embodiment 127, wherein the first binding region of the multispecific antibody binds to PD-1 and the second binding region binds to VEGF, preferably to VEGF-A.

136. The combination according to embodiment 135, binding agent for use according to embodiment 135, antibody-drug conjugate for use according to embodiment 135, or antibody-drug conjugate and binding agent for use according to embodiment 135, wherein the multispecific antibody comprises an anti-VEGF antibody and at least one anti-PD-1 scFv antibody, preferably an anti-VEGF antibody and two anti-PD-1 scFv antibodies.

137. The combination according to embodiment 136, binding agent for use according to embodiment 136, antibody-drug conjugate for use according to embodiment 136, or antibody-drug conjugate and binding agent for use according to embodiment 136, wherein each heavy chain of the anti-VEGF antibody is terminally fused to an anti-PD-1 scFv antibody, preferably via a linker.

138. The combination according to any one of embodiments 135-137, binding agent for use according to any one of embodiments 135-137, antibody-drug conjugate for use according to any one of embodiments 135-137, or antibody-drug conjugate and binding agent for use according to any one of embodiments 135-137, wherein the binding agent comprises:
(A) (1) at least one anti-PD-1 scFv antibody comprising a heavy chain variable region (VH) comprising the HCDR1, HCDR2, and HCDR3 sequences set forth in: SEQ ID NO: 178, 179, and 180, respectively, and a light chain variable region (VL) comprising the LCDR1, LCDR2, and LCDR3 sequences set forth in: SEQ ID NO: 181, 182, and 183, respectively, and (2) an anti-VEGF antibody comprising a heavy chain variable region (VH) comprising the HCDR1, HCDR2, and HCDR3 sequences set forth in: SEQ ID NO: 141, 142, and 143, respectively, and a light chain variable region (VL) comprising the CDR1, CDR2, and CDR3 sequences set forth in: SEQ ID NO: 144, 145, and 140, respectively;
(B) (1) at least one anti-PD-1 scFv antibody comprising a heavy chain variable region (VH) comprising the HCDR1, HCDR2, and HCDR3 sequences set forth in: SEQ ID NO: 184, 185, and 186, respectively, and a light chain variable region (VL) comprising the LCDR1, LCDR2, and LCDR3 sequences set forth in: SEQ ID NO: 187, 188, and 183, respectively, and (2) an anti-VEGF antibody comprising a heavy chain variable region (VH) comprising the HCDR1, HCDR2, and HCDR3 sequences set forth in: SEQ ID NO: 135, 136, and 137, respectively, and a light chain variable region (VL) comprising the LCDR1, LCDR2, and LCDR3 sequences set forth in: SEQ ID NO: 138, 139, and 140, respectively;
(C) (1) at least one anti-PD-1 scFv antibody comprising the amino acid sequence of SEQ ID NO: 189 or an amino acid sequence having at least 80%, at least 85%, at least 90%, at least 95%, at least 96%, at least 97%, at least 98%, at least 99%, or 100% sequence identity to the amino acid sequence SEQ ID NO: 189, wherein the amino acid sequence having at least 80%, at least 85%, at least 90%, at least 95%, at least 96%, at least 97%, at least 98%, at least 99%, or 100% sequence identity to the amino acid sequence SEQ ID NO: 189 preferably comprises the HCDR1, HCDR2, HCDR3, LCDR1, LCDR2, and LCDR3 sequences set forth in: SEQ ID NO: 178, 179, 180, 181, 182, and 183, respectively, and (2) an anti-VEGF antibody comprising a heavy chain variable region (VH) comprising the HCDR1, HCDR2, and HCDR3 sequences set forth in: SEQ ID NO: 141, 142, and 143, respectively, and a light chain variable region (VL) comprising the LCDR1, LCDR2, and LCDR3 sequences set forth in: SEQ ID NO: 144, 145, and 140, respectively; or
(D) (1) at least one anti-PD-1 scFv antibody comprising the amino acid sequence of SEQ ID NO: 189 or an amino acid sequence having at least 80%, at least 85%, at least 90%, at least 95%, at least 96%, at least 97%, at least 98%, at least 99%, or 100% sequence identity to the amino acid sequence SEQ ID NO: 189, wherein the amino acid sequence having at least 80%, at least 85%, at least 90%, at least 95%, at least 96%, at least 97%, at least 98%, at least 99%, or 100% sequence identity to the amino acid sequence SEQ ID NO: 189 preferably comprises the HCDR1, HCDR2, HCDR3, LCDR1, LCDR2, and LCDR3 sequences set forth in: SEQ ID NO: 184, 185, 186, 187, 188, and 183, respectively, and (2) an anti-VEGF antibody comprising a heavy chain variable region (VH) comprising the HCDR1, HCDR2, and HCDR3 sequences set forth in: SEQ ID NO: 135, 136, and 137, respectively, and a light chain variable region (VL) comprising the LCDR1, LCDR2, and LCDR3 sequences set forth in: SEQ ID NO: 138, 139, and 140, respectively.

139. The combination according to any one of embodiments 136-138, binding agent for use according to any one of embodiments 136-138, antibody-drug conjugate for use according to any one of embodiments 136-138, or antibody-drug conjugate and binding agent for use according to any one of embodiments 136-138, wherein the anti-VEGF antibody comprises:
(ii-1d) a heavy chain variable region (VH) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 146, and a light chain variable region (VL) comprising an amino acid sequence having at least 80%, at least 85%, at least 90%, at least 95%, at least 96%, at least 97%, at least 98%, at least 99%, or 100% sequence identity to the amino acid sequence SEQ ID NO: 147;
(ii-1e) a heavy chain variable region (VH) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 146, and a light chain variable region (VL) comprising an amino acid sequence having at least 80%, at least 85%, at least 90%, at least 95%, at least 96%, at least 97%, at least 98%, at least 99%, or 100% sequence identity to the amino acid sequence SEQ ID NO: 147, wherein the VH comprises the HCDR1, HCDR2, and HCDR3 sequences set forth in: SEQ ID NO: 135, 136, and 137, respectively, and the VL comprises the LCDR1, LCDR2, and LCDR3 sequences set forth in: SEQ ID NO: 138, 139, and 140, respectively;
(ii-1f) a heavy chain variable region (VH) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 146, and a light chain variable region (VL) comprising an amino acid sequence having at least 80%, at least 85%, at least 90%, at least 95%, at least 96%, at least 97%, at least 98%, at least 99%, or 100% sequence identity to the amino acid sequence SEQ ID NO: 147, wherein the VH comprises the HCDR1, HCDR2, and HCDR3 sequences set forth in: SEQ ID NO: 141, 142, and 143, respectively, and the VL comprises the LCDR1, LCDR2, and LCDR3 sequences set forth in: SEQ ID NO: 144, 145, and 140, respectively;
(ii-1g) a heavy chain (HC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 151, and a light chain (LC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 152;
(ii-1h) a heavy chain (HC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 151, and a light chain (LC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 152, wherein the HC comprises the HCDR1, HCDR2, and HCDR3 sequences set forth in: SEQ ID NO: 135, 136, and 137, respectively, and the LC comprises the LCDR1, LCDR2, and LCDR3 sequences set forth in: SEQ ID NO: 138, 139, and 140, respectively; or
(ii-1i) a heavy chain (HC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 151, and a light chain (LC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 152, wherein the HC comprises the HCDR1, HCDR2, and HCDR3 sequences set forth in: SEQ ID NO: 141, 142, and 143, respectively, and the LC comprises the LCDR1, LCDR2, and LCDR3 sequences set forth in: SEQ ID NO: 144, 145, and 140, respectively.

140. The combination according to any of embodiments 135-139, binding agent for use according to any one of embodiments 135-139, antibody-drug conjugate for use according to any one of embodiments 135-139, or antibody-drug conjugate and binding agent for use according to any one of embodiments 135-139, wherein the binding agent comprises (A) at least one heavy chain (HC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 159, and (B) at least one light chain (LC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 152, wherein (I) the HC comprises (i) the HCDR1, HCDR2, and HCDR3 sequences set forth in: SEQ ID NO: 135, 136, and 137, respectively, and (ii) the HCDR1, HCDR2, HCDR3, LCDR1, LCDR2, and LCDR3 sequences set forth in: SEQ ID NO: 184, 185, 186, 187, 188, and 183, respectively, or the HC comprises (i′) the HCDR1, HCDR2, and HCDR3 sequences set forth in: SEQ ID NO: 141, 142, and 143, respectively, and (ii′) the HCDR1, HCDR2, HCDR3, LCDR1, LCDR2, and LCDR3 sequences set forth in: SEQ ID NO: 178, 179, 180, 181, 182, and 183, respectively, and (II) the LC comprises the LCDR1, LCDR2, and LCDR3 sequences set forth in: SEQ ID NO: 138, 139, and 140, respectively, or the LCDR1, LCDR2, and LCDR3 sequences set forth in: SEQ ID NO: 144, 145, and 140, respectively.

141. The combination according to any one of embodiments 135-140, binding agent for use according to any one of embodiments 135-140, antibody-drug conjugate for use according to any one of embodiments 135-140, or antibody-drug conjugate and binding agent for use according to any one of embodiments 135-140, wherein the binding agent comprises (A) two heavy chains (HCs) each comprising the amino acid sequence of SEQ ID NO: 159, and (B) two light chains (LCs) each comprising the amino acid sequence of SEQ ID NO: 152.

142. The combination according to embodiment 135, binding agent for use according to embodiment 135, antibody-drug conjugate for use according to embodiment 135, or antibody-drug conjugate and binding agent for use according to embodiment 135, wherein the multispecific antibody comprises an anti-VEGF antibody and at least one anti-PD-1 single domain antibody, preferably an anti-VEGF antibody and at least two anti-PD-1 single domain antibodies, more preferably an anti-VEGF antibody and at least two anti-PD-1 VHHs.

143. The combination according to embodiment 142, binding agent for use according to embodiment 142, antibody-drug conjugate for use according to embodiment 142, or antibody-drug conjugate and binding agent for use according to embodiment 142, wherein each heavy chain of the anti-VEGF antibody is terminally fused to an anti-PD-1 single domain antibody, preferably via a linker.

144. The combination according to any one of embodiments 135, 142 or 143, binding agent for use according to any one of embodiments 135, 142 or 143, antibody-drug conjugate for use according to any one of embodiments 135, 142 or 143, or antibody-drug conjugate and binding agent for use according to any of embodiments 135, 142 or 143, wherein the binding agent comprises:
(A) (1) at least one anti-PD-1 single domain antibody comprising the HCDR1, HCDR2, and HCDR3 sequences set forth in: SEQ ID NO: 110, 111, and 112, respectively, or the HCDR1, HCDR2, and HCDR3 sequences set forth in: SEQ ID NO: 110, 111, and 113, respectively; and (2) an anti-VEGF antibody comprising a heavy chain variable region (VH) comprising the HCDR1, HCDR2, and HCDR3 sequences set forth in: SEQ ID NO: 135, 136, and 137, respectively, and a light chain variable region (VL) comprising the LCDR1, LCDR2, and LCDR3 sequences set forth in: SEQ ID NO: 138, 139, and 140, respectively;
(B) (1) at least one anti-PD-1 single domain antibody comprising a heavy chain variable region (VH) comprising the HCDR1, HCDR2, and HCDR3 sequences set forth in: SEQ ID NO: 114, 115, and 116, respectively, or VH comprising the HCDR1, HCDR2, and HCDR3 sequences set forth in: SEQ ID NO: 114, 115, and 117, respectively; and (2) an anti-VEGF antibody comprising a heavy chain variable region (VH) comprising the HCDR1, HCDR2, and HCDR3 sequences set forth in: SEQ ID NO: 141, 142, and 143, respectively, and a light chain variable region (VL) comprising the LCDR1, LCDR2, and LCDR3 sequences set forth in: SEQ ID NO: 144, 145, and 140, respectively;
(C) (1) at least one anti-PD-1 single domain antibody comprising the amino acid sequence of SEQ ID NO: 118 or an amino acid sequence having at least 80%, at least 85%, at least 90%, at least 95%, at least 96%, at least 97%, at least 98%, at least 99%, or 100% sequence identity to the amino acid sequence SEQ ID NO: 118, wherein the amino acid sequence having at least 80%, at least 85%, at least 90%, at least 95%, at least 96%, at least 97%, at least 98%, at least 99%, or 100% sequence identity to the amino acid sequence SEQ ID NO: 118 preferably comprises the HCDR1, HCDR2, and HCDR3 sequences set forth in: SEQ ID NO: 110, 111, and 112, respectively, or comprises the HCDR1, HCDR2, and HCDR3 sequences set forth in: SEQ ID NO: 114, 115, and 116, respectively, and (2) an anti-VEGF antibody comprising (i) a heavy chain variable region (VH) comprising the HCDR1, HCDR2, and HCDR3 sequences set forth in: SEQ ID NO: 135, 136, and 137, respectively, and a light chain variable region (VL) comprising the LCDR1, LCDR2, and LCDR3 sequences set forth in: SEQ ID NO: 138, 139, and 140, respectively, or (ii) a VH comprising the HCDR1, HCDR2, and HCDR3 sequences set forth in: SEQ ID NO: 141, 142, and 143, respectively, and a VL comprising the LCDR1, LCDR2, and LCDR3 sequences set forth in: SEQ ID NO: 144, 145, and 140, respectively; or
(D) (1) at least one anti-PD-1 single domain antibody comprising the amino acid sequence of SEQ ID NO: 119 or an amino acid sequence having at least 80%, at least 85%, at least 90%, at least 95%, at least 96%, at least 97%, at least 98%, at least 99%, or 100% sequence identity to the amino acid sequence SEQ ID NO: 119, wherein the amino acid sequence having at least 80%, at least 85%, at least 90%, at least 95%, at least 96%, at least 97%, at least 98%, at least 99%, or 100% sequence identity to the amino acid sequence SEQ ID NO: 119 preferably comprises the HCDR1, HCDR2, and HCDR3 sequences set forth in: SEQ ID NO: 110, 111, and 113, respectively, or the HCDR1, HCDR2, and HCDR3 sequences set forth in: SEQ ID NO: 114, 115, and 117, respectively, and (2) an anti-VEGF antibody comprising (i) a heavy chain variable region (VH) comprising the HCDR1, HCDR2, and HCDR3 sequences set forth in: SEQ ID NO: 135, 136, and 137, respectively, and a light chain variable region (VL) comprising the LCDR1, LCDR2, and LCDR3 sequences set forth in: SEQ ID NO: 138, 139, and 140, respectively, or (ii) a VH comprising the HCDR1, HCDR2, and HCDR3 sequences set forth in: SEQ ID NO: 141, 142, and 143, respectively, and a VL comprising the LCDR1, LCDR2, and LCDR3 sequences set forth in: SEQ ID NO: 144, 145, and 140, respectively.

145. The combination according to any one of embodiments 142-144, binding agent for use according to any of embodiments 142-144, antibody-drug conjugate for use according to any of embodiments 142-144, or antibody-drug conjugate and binding agent for use according to any of embodiments 142-144, wherein the anti-VEGF antibody comprises:
(ii-1d) a heavy chain variable region (VH) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 146, and a light chain variable region (VL) comprising an amino acid sequence having at least 80%, at least 85%, at least 90%, at least 95%, at least 96%, at least 97%, at least 98%, at least 99%, or 100% sequence identity to the amino acid sequence SEQ ID NO: 147;
(ii-1e) a heavy chain variable region (VH) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 146, and a light chain variable region (VL) comprising an amino acid sequence having at least 80%, at least 85%, at least 90%, at least 95%, at least 96%, at least 97%, at least 98%, at least 99%, or 100% sequence identity to the amino acid sequence SEQ ID NO: 147, wherein the VH comprises the HCDR1, HCDR2, and HCDR3 sequences set forth in: SEQ ID NO: 135, 136, and 137, respectively, and the VL comprises the LCDR1, LCDR2, and LCDR3 sequences set forth in: SEQ ID NO: 138, 139, and 140, respectively;
(ii-1f) a heavy chain variable region (VH) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 146, and a light chain variable region (VL) comprising an amino acid sequence having at least 80%, at least 85%, at least 90%, at least 95%, at least 96%, at least 97%, at least 98%, at least 99%, or 100% sequence identity to the amino acid sequence SEQ ID NO: 147, wherein the VH comprises the HCDR1, HCDR2, and HCDR3 sequences set forth in: SEQ ID NO: 141, 142, and 143, respectively, and the VL comprises the LCDR1, LCDR2, and LCDR3 sequences set forth in: SEQ ID NO: 144, 145, and 140, respectively;
(ii-1g) a heavy chain (HC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 151, and a light chain (LC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 152;
(ii-1h) a heavy chain (HC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 151, and a light chain (LC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 152, wherein the HC comprises the HCDR1, HCDR2, and HCDR3 sequences set forth in: SEQ ID NO: 135, 136, and 137, respectively, and the LC comprises the LCDR1, LCDR2, and LCDR3 sequences set forth in: SEQ ID NO: 138, 139, and 140, respectively; or (ii-1i) a heavy chain (HC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 151, and a light chain (LC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 152, wherein the HC comprises the HCDR1, HCDR2, and HCDR3 sequences set forth in: SEQ ID NO: 141, 142, and 143, respectively, and the LC comprises the LCDR1, LCDR2, and LCDR3 sequences set forth in: SEQ ID NO: 144, 145, and 140, respectively.

146. The combination according to any one of embodiments 135 or 142-145, binding agent for use according to any one of embodiments 135 or 142-145, antibody-drug conjugate for use according to any one of embodiments 135 or 142-145, or antibody-drug conjugate and binding agent for use according to any one of embodiments 135 or 142-145, wherein the binding agent comprises (A) at least one heavy chain (HC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 160, and (B) at least one light chain (LC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 152, wherein (I) the HC comprises (i) the HCDR1, HCDR2, and HCDR3 sequences set forth in: SEQ ID NO: 135, 136, and 137, respectively, and (ii) the HCDR1, HCDR2, and HCDR3 sequences set forth in: SEQ ID NO: 110, 111, and 112, respectively, or the HC comprises (i′) the HCDR1, HCDR2, and HCDR3 sequences set forth in: SEQ ID NO: 141, 142, and 143, respectively, and (ii′) the HCDR1, HCDR2, and HCDR3, sequences set forth in: SEQ ID NO: 114, 115, and 116, respectively, and (II) the LC comprises the LCDR1, LCDR2, and LCDR3 sequences set forth in: SEQ ID NO: 138, 139, and 140, respectively, or the LCDR1, LCDR2, and LCDR3 sequences set forth in: SEQ ID NO: 144, 145, and 140, respectively.

147. The combination according to any one of embodiments 135 or 142-145, binding agent for use according to any one of embodiments 135 or 142-145, antibody-drug conjugate for use according to any one of embodiments 135 or 142-145, or antibody-drug conjugate and binding agent for use according to any one of embodiments 135 or 142-145, wherein the binding agent comprises (A) at least one heavy chain (HC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 161, and (B) at least one light chain (LC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 152, wherein (I) the HC comprises (i) the HCDR1, HCDR2, and HCDR3 sequences set forth in: SEQ ID NO: 135, 136, and 137, respectively, and (ii) the HCDR1, HCDR2, and HCDR3 sequences set forth in: SEQ ID NO: 110, 111, and 112, respectively, or the HC comprises (i′) the HCDR1, HCDR2, and HCDR3 sequences set forth in: SEQ ID NO: 141, 142, and 143, respectively, and (ii′) the HCDR1, HCDR2, and HCDR3, sequences set forth in: SEQ ID NO: 114, 115, and 116, respectively, and (II) the LC comprises the LCDR1, LCDR2, and LCDR3 sequences set forth in: SEQ ID NO: 138, 139, and 140, respectively, or the LCDR1, LCDR2, and LCDR3 sequences set forth in: SEQ ID NO: 144, 145, and 140, respectively.

148. The combination according to any one of embodiments 135 or 142-147, binding agent for use according to any one of embodiments 135 or 142-147, antibody-drug conjugate for use according to any one of embodiments 135 or 142-147, or antibody-drug conjugate and binding agent for use according to any of embodiments 135 or 142-147, wherein the binding agent comprises (A) at least one heavy chain (HC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 162, and (B) at least one light chain (LC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 152, wherein (I) the HC comprises (i) the HCDR1, HCDR2, and HCDR3 sequences set forth in: SEQ ID NO: 135, 136, and 137, respectively, and (ii) the HCDR1, HCDR2, and HCDR3 sequences set forth in: SEQ ID NO: 110, 111, and 113, respectively, or the HC comprises (i′) the HCDR1, HCDR2, and HCDR3 sequences set forth in: SEQ ID NO: 141, 142, and 143, respectively, and (ii′) the HCDR1, HCDR2, and HCDR3, sequences set forth in: SEQ ID NO: 114, 115, and 117, respectively, and (II) the LC comprises the LCDR1, LCDR2, and LCDR3 sequences set forth in: SEQ ID NO: 138, 139, and 140, respectively, or the LCDR1, LCDR2, and LCDR3 sequences set forth in: SEQ ID NO: 144, 145, and 140, respectively.

149. The combination according to any one of embodiments 135 or 142-148, binding agent for use according to any one of embodiments 135 or 142-148, antibody-drug conjugate for use according to any one of embodiments 135 or 142-148, or antibody-drug conjugate and binding agent for use according to any one of embodiments 135 or 142-148, wherein the binding agent comprises (A) two heavy chains (HCs) each comprising the amino acid sequence of SEQ ID NO: 160, the amino acid sequence of SEQ ID NO: 161, or the amino acid sequence of SEQ ID NO: 162, and (B) two light chains (LCs) each comprising the amino acid sequence of SEQ ID NO: 152.

150. The combination according to embodiment 127, binding agent for use according to embodiment 127, antibody-drug conjugate for use according to embodiment 127, or antibody-drug conjugate and binding agent for use according to embodiment 127, wherein the first binding region of the multispecific antibody binds to PD-L1 and the second binding region comprises at least one VEGFR domain.

151. The combination according to embodiment 150, binding agent for use according to embodiment 150, antibody-drug conjugate for use according to embodiment 150, or antibody-drug conjugate and binding agent for use according to embodiment 150, wherein the multispecific antibody comprises a fusion protein comprising an anti-PD-L1 antibody and the at least one VEGFR domain.

152. The combination according to embodiment 151, binding agent for use according to embodiment 151, antibody-drug conjugate for use according to embodiment 151, or antibody-drug conjugate and binding agent for use according to embodiment 151, wherein:
(i) the at least one VEGR domain is terminally fused to the heavy chain of the anti-PD-L1 antibody, preferably via a linker;
(ii) the anti-PD-L1 antibody comprises a full-length anti-PD-L1 antibody; and/or
(iii) the anti-PD-L1 antibody is a humanized antibody.

153. The combination according to any one of embodiments 150-152, binding agent for use according to any one of embodiments 150-152, antibody-drug conjugate for use according to any one of embodiments 150-152, or antibody-drug conjugate and binding agent for use according to any one of embodiments 150-152, wherein the binding agent comprises:
(A) (1) a heavy chain variable region (VH) comprising the HCDR1, HCDR2, and HCDR3 sequences of SEQ ID NO: 133, and/or a light chain variable region (VL) comprising the LCDR1, LCDR2, and LCDR3 sequences of SEQ ID NO: 134; and (2) at least one VEGFR domain comprising the amino acid sequence of SEQ ID NO:155;
(B) (1) a heavy chain variable region (VH) comprising the HCDR1, HCDR2, and HCDR3 sequences set forth in: SEQ ID NO: 127, 128, and 129, respectively, and/or a light chain variable region (VL) comprising the LCDR1, LCDR2, and LCDR3 sequences set forth in: SEQ ID NO: 130, 131, and 132, respectively; and (2) at least one VEGFR domain comprising the amino acid sequence of SEQ ID NO:155; or
(C) (1) a heavy chain variable region (VH) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 133, and a light chain variable region (VL) comprising an amino acid sequence having at least 80%, at least 85%, at least 90%, at least 95%, at least 96%, at least 97%, at least 98%, at least 99%, or 100% sequence identity to the amino acid sequence SEQ ID NO: 134, wherein preferably the VH comprises the HCDR1, HCDR2, and HCDR3 sequences set forth in: SEQ ID NO: 127, 128, and 129, respectively, and the VL comprises the LCDR1, LCDR2, and LCDR3 sequences set forth in: SEQ ID NO: 130, 131, and 132, respectively; and (2) at least one VEGFR domain comprising the amino acid sequence of SEQ ID NO: 155.

154. The combination according to any one of embodiments 150-153, binding agent for use according to any one of embodiments 150-153, antibody-drug conjugate for use according to any one of embodiments 150-153, or antibody-drug conjugate and binding agent for use according to any one of embodiments 150-153, wherein the binding agent comprises (A) at least one heavy chain (HC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 163, and (B) at least one light chain (LC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 164, wherein the HC comprises the HCDR1, HCDR2, and HCDR3 sequences set forth in: SEQ ID NO: 127, 128, and 129, respectively, and the LC comprises the LCDR1, LCDR2, and LCDR3 sequences set forth in: SEQ ID NO: 130, 131, and 132, respectively.

155. The combination according to any one of embodiments 150-153, binding agent for use according to any one of embodiments 150-153, antibody-drug conjugate for use according to any one of embodiments 150-153, or antibody-drug conjugate and binding agent for use according to any one of embodiments 150-153, wherein the binding agent comprises (A) at least one heavy chain (HC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 165, and (B) at least one light chain (LC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 164, wherein the HC comprises the HCDR1, HCDR2, and HCDR3 sequences set forth in: SEQ ID NO: 127, 128, and 129, respectively, and the LC comprises the LCDR1, LCDR2, and LCDR3 sequences set forth in: SEQ ID NO: 130, 131, and 132, respectively.

156. The combination according to any one of embodiments 150-155, binding agent for use according to any one of embodiments 150-155, antibody-drug conjugate for use according to any one of embodiments 150-155, or antibody-drug conjugate and binding agent for use according to any one of embodiments 150-155, wherein the binding agent comprises (A) two heavy chains (HCs) each comprising the amino acid sequence of SEQ ID NO: 163 or the amino acid sequence of SEQ ID NO: 165, and (B) two light chains (LCs) each comprising the amino acid sequence of SEQ ID NO: 164.

157. The combination according to embodiment 127, binding agent for use according to embodiment 127, antibody-drug conjugate for use according to embodiment 127, or antibody-drug conjugate and binding agent for use according to embodiment 127, wherein the first binding region of the multispecific antibody binds to PD-1 and the second binding region of the multispecific antibody binds to VEGFR, preferably to VEGFR2, preferably wherein the multispecific antibody is an antibody or binding fragment thereof that specifically binds to both PD-1 and VEGFR, preferably VEGFR2.

158. The combination according to embodiment 157, binding agent for use according to embodiment 157, antibody-drug conjugate for use according to embodiment 157, or antibody-drug conjugate and binding agent for use according to embodiment 157, wherein the multispecific antibody comprises a VH region that comprises an IGHV3-7 human germline scaffold amino acid sequence into which a set of corresponding HCDR1, HCDR2 and HCDR3 amino acid sequences have been inserted and a VL region that comprises an IGKV1-human germline scaffold amino acid sequence into which a set of corresponding LCDR1, LCDR2 and LCDR3 amino acid sequences have been inserted.

159. The combination according to embodiment 157 or 158, binding agent for use according to embodiment 157 or 158, antibody-drug conjugate for use according to embodiment 157 or 158, or antibody-drug conjugate and binding agent for use according to embodiment 157 or 158, wherein the multispecific antibody comprises an IgG1, IgG2, IgG3, IgG4, IgAQ1 or IgA2 immunoglobulin constant region, preferably wherein the multispecific antibody comprises an immunologically inert immunoglobulin constant region.

160. The combination according to any one of embodiments 157-159, binding agent for use according to any one of embodiments 157-159, antibody-drug conjugate for use according to any one of embodiments 157-159, or antibody-drug conjugate and binding agent for use according to any one of embodiments 157-159, wherein the multispecific antibody comprises (I) a VH comprising: (i) a HCDR1 comprising or consisting of the amino acid sequence of SEQ ID NO: 330; (ii) a HCDR2 comprising or consisting of the amino acid sequence of SEQ ID NO: 331; and (iii) a HCDR3 comprising or consisting of the amino acid sequence of SEQ ID NO: 332, and/or (II) a VL comprising: (i) a LCDR1 comprising or consisting of the amino acid sequence of SEQ ID NO: 333; (ii) a LCDR2 comprising or consisting of the amino acid sequence of SEQ ID NO: 334, (ii′) a LCDR2 comprising or consisting of the amino acid sequence of SEQ ID NO: 338, (ii″) a LCDR2 comprising or consisting of the amino acid sequence of SEQ ID NO: 340, or (ii′») a LCDR2 comprising or consisting of the amino acid sequence of SEQ ID NO: 342; and (iii) a LCDR3 comprising or consisting of the amino acid sequence of SEQ ID NO: 335.

161. The combination according to any one of embodiments 157-160, binding agent for use according to any one of embodiments 157-160, antibody-drug conjugate for use according to any one of embodiments 157-160, or antibody-drug conjugate and binding agent for use according to any one of embodiments 157-160, wherein the multispecific antibody comprises a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 336; and/or a VL comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 337, 339, 341, or 343.

162. The combination according to any one of embodiments 157-161, binding agent for use according to any one of embodiments 157-161, antibody-drug conjugate for use according to any one of embodiments 157-161, or antibody-drug conjugate and binding agent for use according to any one of embodiments 157-161, wherein the multispecific antibody comprises a VH comprising or consisting of the amino acid sequence of SEQ ID NO: 336; and/or a VL comprising or consisting of the amino acid sequence of SEQ ID NO: 337, 339, 341, or 343.

163. The combination according to any one of embodiments 1 or 5-162, binding agent for use according to any one of embodiments 2 or 5-162, antibody-drug conjugate for use according to any one of embodiments 3 or 5-162, or antibody-drug conjugate and binding agent for use according to any one of embodiments 4-162, wherein the binding agent is a multispecific antibody, preferably a bispecific antibody that comprises (a) an antibody or fragment thereof that specifically binds to PD-L1 and (b) an antibody or fragment thereof that specifically binds to VEGF.

164. The combination according to embodiment 163, binding agent for use according to embodiment 163, antibody-drug conjugate for use according to embodiment 163, or antibody-drug conjugate and binding agent for use according to embodiment 163, wherein the anti-PD-L1 antibody or fragment thereof and/or the anti-VEGF antibody or fragment thereof comprises a single chain antibody, a humanized antibody, a chimeric antibody or diabody antibody, preferably a single chain antibody, more preferably a single chain antibody selected from an Fab, an Fab′, an F(ab′)2, an Fv, an ScFv, an Fab′-SH, an sdAb, and a VHH, most preferably a VHH.

165. The combination according to embodiment 163 or 164, binding agent for use according to embodiment 163 or 164, antibody-drug conjugate for use according to embodiment 163 or 164, or antibody-drug conjugate and binding agent for use according to embodiment 163 or 164, wherein the multispecific antibody, preferably the bispecific antibody comprises an anti-PD-L1 single domain antibody.

166. The combination according to any one of embodiments 163-165, binding agent for use according to any one of embodiments 163-165, antibody-drug conjugate for use according to any one of embodiments 163-165, or antibody-drug conjugate and binding agent for use according to any one of embodiments 163-165, wherein the multispecific antibody, preferably the bispecific antibody comprises two anti-PD-L1 single domain antibodies, preferably two VHHs, preferably wherein each VHH is fused to the C-terminus of an anti-VEGF antibody.

167. The combination according to any one of embodiments 163-166, binding agent for use according to any one of embodiments 163-166, antibody-drug conjugate for use according to any one of embodiments 163-166, or antibody-drug conjugate and binding agent for use according to any one of embodiments 163-166, wherein the multispecific antibody, preferably the bispecific antibody comprises an anti-PD-L1 single domain antibody comprising:
(i) (i) a HCDR1 comprising or consisting of the amino acid sequence shown in SEQ ID NO: 84, (ii) a HCDR2 comprising or consisting of the amino acid sequence shown in SEQ ID NO: 85, and (iii) a HCDR3 comprising or consisting of the amino acid sequence shown in SEQ ID NO: 86, wherein the CDRs are defined according to the IMGT numbering system; and/or
(ii) (i) a HCDR1 comprising or consisting of the amino acid sequence shown in SEQ ID NO: 81, (ii) a HCDR2 comprising or consisting of the amino acid sequence shown in SEQ ID NO: 82, and (iii) a HCDR3 comprising or consisting of the amino acid sequence shown in SEQ ID NO: 83, wherein the CDRs are defined according to the Kabat numbering system.

168. The combination according to any one of embodiments 163-167, binding agent for use according to any one of embodiments 163-167, antibody-drug conjugate for use according to any one of embodiments 163-167, or antibody-drug conjugate and binding agent for use according to any one of embodiments 163-167, wherein the multispecific antibody, preferably the bispecific antibody comprises an anti-PD-L1 single domain antibody comprising or consisting of the amino acid sequence of SEQ ID NO: 87 or an amino acid sequence having at least 80%, 85%, 90%, 95%, 96%, 97%, 98%, or 99% identity to SEQ ID NO: 87, preferably wherein the antibody or fragment thereof that specifically binds to PD-L1 comprises: (i) a HCDR1 comprising or consisting of the amino acid sequence shown in SEQ ID NO: 81, (ii) a HCDR2 comprising or consisting of the amino acid sequence shown in SEQ ID NO: 82, and (iii) a HCDR3 comprising or consisting of the amino acid sequence shown in SEQ ID NO: 83, wherein the CDRs are defined according to the Kabat numbering system; and/or (i) a HCDR1 comprising or consisting of the amino acid sequence shown in SEQ ID NO: 84, (ii) a HCDR2 comprising or consisting of the amino acid sequence shown in SEQ ID NO: 85, and (iii) a HCDR3 comprising or consisting of the amino acid sequence shown in SEQ ID NO: 86, wherein the CDRs are defined according to the IMGT numbering system.

169. The combination according to any one of embodiments 163-168, binding agent for use according to any one of embodiments 163-168, antibody-drug conjugate for use according to any one of embodiments 163-168, or antibody-drug conjugate and binding agent for use according to any one of embodiments 163-168, wherein the multispecific antibody, preferably the bispecific antibody comprises an anti-VEGF antibody or fragment thereof comprising a constant region derived from a human antibody, preferably the constant region is selected from the constant region of human IgG1, IgG2, IgG3 or IgG4.

170. The combination according to any one of embodiments 163-169, binding agent for use according to any one of embodiments 163-169, antibody-drug conjugate for use according to any one of embodiments 163-169, or antibody-drug conjugate and binding agent for use according to any one of embodiments 163-169, wherein the multispecific antibody, preferably the bispecific antibody comprises an anti-VEGF antibody or fragment thereof comprising an Fc region comprising one or more mutations which reduce or abolish immune effect functions, preferably wherein the anti-VEGF antibody or fragment thereof comprises an IgG1 Fc region comprising L234A and L235A mutations.

171. The combination according to any one of embodiments 163-170, binding agent for use according to any one of embodiments 163-170, antibody-drug conjugate for use according to any one of embodiments 163-170, or antibody-drug conjugate and binding agent for use according to any one of embodiments 163-170, wherein the multispecific antibody, preferably the bispecific antibody comprises an anti-VEGF antibody or fragment thereof comprising an IgG1 Fc region, preferably comprising or consisting of the amino acid sequence shown in SEQ ID NO: 103 or an amino acid sequence having at least 80%, 85%, 90%, 95%, 96%, 97%, 98%, or 99% identity to the amino acid sequence shown in SEQ ID NO: 103.

172. The combination according to any one of embodiments 163-171, binding agent for use according to any one of embodiments 163-171, antibody-drug conjugate for use according to any one of embodiments 163-171, or antibody-drug conjugate and binding agent for use according to any one of embodiments 163-171, wherein the multispecific antibody, preferably the bispecific antibody comprises an anti-VEGF antibody or fragment thereof that specifically binds to VEGF-A.

173. The combination according to any one of embodiments 163-172, binding agent for use according to any one of embodiments 163-172, antibody-drug conjugate for use according to any one of embodiments 163-172, or antibody-drug conjugate and binding agent for use according to any one of embodiments 163-172, wherein the multispecific antibody, preferably the bispecific antibody comprises an anti-VEGF antibody or fragment thereof comprising:
(i) a heavy chain variable region comprising: (i) a HCDR1 comprising or consisting of the amino acid sequence shown in SEQ ID NO: 88, (ii) a HCDR2 comprising or consisting of the amino acid sequence shown in SEQ ID NO: 89, and (iii) a HCDR3 comprising or consisting of the amino acid sequence shown in SEQ ID NO: 90; and a light chain variable region comprising: (i) a LCDR1 comprising or consisting of the amino acid sequence shown in SEQ ID NO: 91, (ii) a LCDR2 comprising or consisting of the amino acid sequence shown in SEQ ID NO: 92, and (iii) a LCDR3 comprising or consisting of the amino acid sequence shown in SEQ ID NO: 93, wherein the CDRs are defined according to the Kabat numbering system; and/or
(ii) a heavy chain variable region comprising: (i) a HCDR1 comprising or consisting of the amino acid sequence shown in SEQ ID NO: 94, (ii) a HCDR2 comprising or consisting of the amino acid sequence shown in SEQ ID NO: 95, and (iii) a HCDR3 comprising or consisting of the amino acid sequence shown in SEQ ID NO: 96; and a light chain variable region comprising: (i) a LCDR1 comprising or consisting of the amino acid sequence shown in SEQ ID NO: 97, (ii) a LCDR2 comprising or consisting of the amino acid sequence shown in SEQ ID NO: 98, and (iii) a LCDR3 comprising or consisting of the amino acid sequence shown in SEQ ID NO: 99, wherein the CDRs are defined according to the IMGT numbering system.

174. The combination according to any one of embodiments 163-173, binding agent for use according to any one of embodiments 163-173, antibody-drug conjugate for use according to any one of embodiments 163-173, or antibody-drug conjugate and binding agent for use according to any one of embodiments 163-173, wherein the multispecific antibody, preferably the bispecific antibody comprises an anti-VEGF antibody or fragment thereof comprising: a heavy chain variable region comprising or consisting of the amino acid sequence shown in SEQ ID NO: 100 or an amino acid sequence having at least 80%, 85%, 90%, 95%, 96%, 97%, 98%, or 99% identity thereto; and a light chain variable region comprising or consisting of the amino acid sequence shown in SEQ ID NO: 101 or an amino acid sequence having at least 80%, 85%, 90%, 95%, 96%, 97%, 98%, or 99% identity thereto, preferably wherein the antibody or fragment thereof that specifically binds to VEGF comprises: (i) a HCDR1 comprising or consisting of the amino acid sequence shown in SEQ ID NO: 88, (ii) a HCDR2 comprising or consisting of the amino acid sequence shown in SEQ ID NO: 89, (iii) a HCDR3 comprising or consisting of the amino acid sequence shown in SEQ ID NO: 90; (i) a LCDR1 comprising or consisting of the amino acid sequence shown in SEQ ID NO: 91, (ii) a LCDR2 comprising or consisting of the amino acid sequence shown in SEQ ID NO: 92, and (iii) a LCDR3 comprising or consisting of the amino acid sequence shown in SEQ ID NO: 93, wherein the CDRs are defined according to the Kabat numbering system; and/or (i) a HCDR1 comprising or consisting of the amino acid sequence shown in SEQ ID NO: 94, (ii) a HCDR2 comprising or consisting of the amino acid sequence shown in SEQ ID NO: 95, (iii) a HCDR3 comprising or consisting of the amino acid sequence shown in SEQ ID NO: 96; (i) a LCDR1 comprising or consisting of the amino acid sequence shown in SEQ ID NO: 97, (ii) a LCDR2 comprising or consisting of the amino acid sequence shown in SEQ ID NO: 98, and (iii) a LCDR3 comprising or consisting of the amino acid sequence shown in SEQ ID NO: 99, wherein the CDRs are defined according to the IMGT numbering system.

175. The combination according to any one of embodiments 163-174, binding agent for use according to any one of embodiments 163-174, antibody-drug conjugate for use according to any one of embodiments 163-174, or antibody-drug conjugate and binding agent for use according to any one of embodiments 163-174, wherein the multispecific antibody, preferably the bispecific antibody comprises an anti-VEGF antibody or fragment thereof comprising: a heavy chain comprising or consisting of the amino acid sequence shown in SEQ ID NO: 105 or an amino acid sequence having at least 80%, 85%, 90%, 95%, 96%, 97%, 98%, or 99% identity thereto; and a light chain comprising or consisting of the amino acid sequence shown in SEQ ID NO: 106 or an amino acid sequence having at least 80%, 85%, 90%, 95%, 96%, 97%, 98%, or 99% identity thereto, preferably wherein the antibody or fragment thereof that specifically binds to VEGF comprises: (i) a HCDR1 comprising or consisting of the amino acid sequence shown in SEQ ID NO: 88, (ii) a HCDR2 comprising or consisting of the amino acid sequence shown in SEQ ID NO: 89, (iii) a HCDR3 comprising or consisting of the amino acid sequence shown in SEQ ID NO: 90; (i) a LCDR1 comprising or consisting of the amino acid sequence shown in SEQ ID NO: 91, (ii) a LCDR2 comprising or consisting of the amino acid sequence shown in SEQ ID NO: 92, and (iii) a LCDR3 comprising or consisting of the amino acid sequence shown in SEQ ID NO: 93, wherein the CDRs are defined according to the Kabat numbering system; and/or (i) a HCDR1 comprising or consisting of the amino acid sequence shown in SEQ ID NO: 94, (ii) a HCDR2 comprising or consisting of the amino acid sequence shown in SEQ ID NO: 95, (iii) a HCDR3 comprising or consisting of the amino acid sequence shown in SEQ ID NO: 96; (i) a LCDR1 comprising or consisting of the amino acid sequence shown in SEQ ID NO: 97, (ii) a LCDR2 comprising or consisting of the amino acid sequence shown in SEQ ID NO: 98, and (iii) a LCDR3 comprising or consisting of the amino acid sequence shown in SEQ ID NO: 99, wherein the CDRs are defined according to the IMGT numbering system.

176. The combination according to any one of embodiments 163-175, binding agent for use according to any one of embodiments 163-175, antibody-drug conjugate for use according to any one of embodiments 163-175, or antibody-drug conjugate and binding agent for use according to any one of embodiments 163-175, wherein the multispecific antibody, preferably the bispecific antibody comprises bevacizumab or a fragment thereof.

177. The combination according to any one of embodiments 1 or 5-163, binding agent for use according to one of embodiments 2 or 5-163, antibody-drug conjugate for use according to any one of embodiments 3 or 5-163, or antibody-drug conjugate and binding agent for use according to any one of embodiments 4-163, wherein the binding agent comprises: a heavy chain comprising or consisting of the amino acid sequence of SEQ ID NO: 108 or an amino acid sequence having at least 80%, 85%, 90%, 95%, 96%, 97%, 98%, or 99% identity thereto; and a light chain comprising or consisting of the amino acid sequence of SEQ ID NO: 109 or an amino acid sequence having at least 80%, 85%, 90%, 95%, 96%, 97%, 98%, or 99% identity thereto, preferably wherein the binding agent comprises: (a) (i) a HCDR1 comprising or consisting of the amino acid sequence shown in SEQ ID NO: 81, (ii) a HCDR2 comprising or consisting of the amino acid sequence shown in SEQ ID NO: 82, and (iii) a HCDR3 comprising or consisting of the amino acid sequence shown in SEQ ID NO: 83, wherein the CDRs are defined according to the Kabat numbering system; and/or (i) a HCDR1 comprising or consisting of the amino acid sequence shown in SEQ ID NO: 84, (ii) a HCDR2 comprising or consisting of the amino acid sequence shown in SEQ ID NO: 85, and (iii) a HCDR3 comprising or consisting of the amino acid sequence shown in SEQ ID NO: 86, wherein the CDRs are defined according to the IMGT numbering system; and (b) (i) a HCDR1 comprising or consisting of the amino acid sequence shown in SEQ ID NO: 88, (ii) a HCDR2 comprising or consisting of the amino acid sequence shown in SEQ ID NO: 89, (iii) a HCDR3 comprising or consisting of the amino acid sequence shown in SEQ ID NO: 90; (i) a LCDR1 comprising or consisting of the amino acid sequence shown in SEQ ID NO: 91, (ii) a LCDR2 comprising or consisting of the amino acid sequence shown in SEQ ID NO: 92, and (iii) a LCDR3 comprising or consisting of the amino acid sequence shown in SEQ ID NO: 93, wherein the CDRs are defined according to the Kabat numbering system; and/or (i) a HCDR1 comprising or consisting of the amino acid sequence shown in SEQ ID NO: 94, (ii) a HCDR2 comprising or consisting of the amino acid sequence shown in SEQ ID NO: 95, (iii) a HCDR3 comprising or consisting of the amino acid sequence shown in SEQ ID NO: 96; (i) a LCDR1 comprising or consisting of the amino acid sequence shown in SEQ ID NO: 97, (ii) a LCDR2 comprising or consisting of the amino acid sequence shown in SEQ ID NO: 98, and (iii) a LCDR3 comprising or consisting of the amino acid sequence shown in SEQ ID NO: 99, wherein the CDRs are defined according to the IMGT numbering system.

178. The combination according to any one of embodiments 1 or 5-177, binding agent for use according to any one of embodiments 2 or 5-177, antibody-drug conjugate for use according to any one of embodiments 3 or 5-177, or antibody-drug conjugate and binding agent for use according to any one of embodiments 4-177, wherein the binding agent is formulated as a liquid, a solid, or a combination thereof, preferably as a liquid.

179. The combination according to any one of embodiments 1 or 5-178, binding agent for use according to any one of embodiments 2 or 5-178, antibody-drug conjugate for use according to any one of embodiments 3 or 5-178, or antibody-drug conjugate and binding agent for use according to any one of one of embodiments 4-178, wherein the binding agent is formulated for injection or infusion, preferably wherein the binding agent is formulated for intravenous administration.

180. The combination according to any one of embodiments 1 or 5-179, binding agent for use according to any one of embodiments 2 or 5-179, antibody-drug conjugate for use according to any one of embodiments 3 or 5-179, or antibody-drug conjugate and binding agent for use according to any one of embodiments 4-179, wherein the antibody-drug conjugate is formulated as a liquid, a solid, or a combination thereof, preferably as a liquid.

181. The combination according to any one of embodiments 1 or 5-180, binding agent for use according to any one of embodiments 2 or 5-180, antibody-drug conjugate for use according to any one of embodiments 3 or 5-180, or antibody-drug conjugate and binding agent for use according to any one of embodiments 4-180, wherein the antibody-drug conjugate is formulated for injection or infusion, preferably wherein the antibody-drug conjugate is formulated for intravenous administration.

182. The combination according to any one of embodiments 1 or 5-181, binding agent for use according to any one of embodiments 2 or 5-181, antibody-drug conjugate for use according to any one of embodiments 3 or 5-181, or antibody-drug conjugate and binding agent for use according to any one of embodiments 4-181, wherein the binding agent is administered in the form of one or several nucleic acids encoding the binding agent.

183 The binding agent for use according to any one of embodiments 2 or 5-182, antibody-drug conjugate for use according to any one of embodiments 3 or 5-182, or antibody-drug conjugate and binding agent for use according to any one of embodiments 4-182, wherein the binding agent and the antibody-drug conjugate are separately administered, preferably wherein the binding agent is to be administered or is administered prior to, simultaneously with, or after administration of the antibody-drug conjugate.

184. The binding agent for use according to any one of embodiments 2 or 5-183, antibody-drug conjugate for use according to any one of embodiments 3 or 5-183, or antibody-drug conjugate and binding agent for use according to any one of embodiments 4-183, wherein a dose of the binding agent and a dose of the antibody-drug conjugate are administered concurrently or consecutively.

185. The binding agent for use according to any one of embodiments 2 or 5-184, antibody-drug conjugate for use according to any one of embodiments 3 or 5-184, or antibody-drug conjugate and binding agent for use according to any one of embodiments 4-184, wherein the binding agent dose administered ranges from 0.1 mg/kg to 100 mg/kg body weight, preferably 1 mg/kg to 60 mg/kg body weight.

186. The binding agent for use according to any one of embodiments 2 or 5-185, antibody-drug conjugate for use according to any one of embodiments 3 or 5-185, or antibody-drug conjugate and binding agent for use according to any one of embodiments 4-185, wherein the antibody-drug conjugate dosage administered ranges from 0.5 mg/kg to 40 mg/kg body weight, preferably 1 mg/kg to 30 mg/kg body weight.

187. The binding agent for use according to any one of embodiments 2 or 5-186, antibody-drug conjugate for use according to any one of embodiments 3 or 5-186, or antibody-drug conjugate and binding agent for use according to any one of embodiments 4-186, wherein the binding agent and/or the antibody-drug conjugate is administered systemically, preferably intravenously, preferably wherein the binding agent and the antibody-drug conjugate are administered systemically, preferably intravenously.

188. The binding agent for use according to any one of embodiments 2 or 5-187, antibody-drug conjugate for use according to any one of embodiments 3 or 5-187, or antibody-drug conjugate and binding agent for use according to any one of embodiments 4-187, wherein the binding agent and/or the antibody-drug conjugate is administered via an IV injection or IV infusion.

189. The binding agent for use according to any one of embodiments 2 or 5-188, antibody-drug conjugate for use according to any one of embodiments 3 or 5-188, or antibody-drug conjugate and binding agent for use according to any one of embodiments 4-188, wherein the subject has not been previously treated for cancer.

190. The binding agent for use according to any one of embodiments 2 or 5-188, antibody-drug conjugate for use according to any one of embodiments 3 or 5-188, or antibody-drug conjugate and binding agent for use according to any one of embodiments 4-188, wherein the subject has been previously treated for cancer.

191. The binding agent for use according to any one of embodiments 2 or 5-190, antibody-drug conjugate for use according to any one of embodiments 3 or 5-190, or antibody-drug conjugate and binding agent for use according to any one of embodiments 4-190, wherein the binding agent is administered every 6 weeks, preferably every 4 weeks, more preferably every 3 weeks or every 2 weeks.

192. The binding agent for use according to any one of embodiments 2 or 5-191, antibody-drug conjugate for use according to any one of embodiments 3 or 5-191, or antibody-drug conjugate and binding agent for use according to any one of embodiments 4-191, wherein overall survival is increased in the subject compared to the antibody-drug conjugate or the binding agent treatment alone.

193. The binding agent for use according to any one of embodiments 2 or 5-192, antibody-drug conjugate for use according to any one of embodiments 3 or 5-192, or antibody-drug conjugate and binding agent for use according to any one of embodiments 4-192, wherein median progression-free survival is increased in the subject compared to the antibody-drug conjugate or the binding agent treatment alone.

194. The binding agent for use according to any one of embodiments 2 or 5-193, antibody-drug conjugate for use according to any one of embodiments 3 or 5-193, or antibody-drug conjugate and binding agent for use according to any one of embodiments 4-193, wherein the cancer is positive for one or more target(s) expressed on the surface of a tumour cell.

195. The binding agent for use according to any one of embodiments 2 or 5-194, antibody-drug conjugate for use according to any one of embodiments 3 or 5-194, or antibody-drug conjugate and binding agent for use according to any one of embodiments 4-194, wherein the cancer is a solid tumour.

196. The binding agent for use according to any one of embodiments 2 or 5-194, antibody-drug conjugate for use according to any one of embodiments 3 or 5-194, or antibody-drug conjugate and binding agent for use according to any one of embodiments 4-194, wherein the cancer is selected from esophageal cancer (e.g., esophageal adenocarcinoma and esophageal squamous cell carcinoma), brain tumour, lung cancer, small cell lung cancer (SCLC), non-small cell lung cancer (NSCLC) with or without actionable oncogenic alterations (AGA) (e.g., small cell lung cancer and non-small cell lung cancer), squamous cell carcinoma, bladder cancer, stomach cancer, ovarian cancer, peritoneal cancer, pancreatic cancer, breast cancer, triple-negative breast cancer (TNBC), head and neck cancer, cervical cancer, endometrial cancer, gastro-intestinal cancer, colorectal cancer, liver cancer, kidney cancer, urothelial cancer, solid tumour, non-Hodgkin lymphoma, central nervous system tumour (e.g., neuroglioma, glioblastoma multiforme, glioma or sarcoma), adenocarcinoma, prostate cancer, thyroid cancer, soft tissue sarcoma, vulvar cancer or gallbladder cancer.

197. The binding agent for use according to any one of embodiments 2 or 5-195, antibody-drug conjugate for use according to any one of embodiments 3 or 5-195, or antibody-drug conjugate and binding agent for use according to any one of embodiments 4-195, wherein the cancer is selected from colorectal cancer (e.g., colon cancer), lung cancer (e.g., non-small cell lung cancer), breast cancer, prostate cancer, soft tissue sarcoma, vulvar cancer, and gallbladder cancer.

198. The binding agent for use according to any one of embodiments 2 or 5-195, antibody-drug conjugate for use according to any one of embodiments 3 or 5-195, or antibody-drug conjugate and binding agent for use according to any one of embodiments 4-195, wherein the cancer is selected from lung cancer, kidney cancer, urinary tract carcinoma, colorectal cancer, prostatic cancer, glioblastoma multiforme, ovarian cancer, pancreatic cancer, breast cancer, melanoma, liver cancer, bladder cancer, stomach cancer, esophageal cancer, soft tissue sarcoma, vulvar cancer and gallbladder cancer.

199. The binding agent for use according to any one of embodiments 2 or 5-195, antibody-drug conjugate for use according to any one of embodiments 3 or 5-195, or antibody-drug conjugate and binding agent for use according to any one of embodiments 4-195, wherein the cancer is selected from breast cancer, lung cancer, gastro-intestinal cancer, and adenocarcinoma.

200. The binding agent for use according to any one of embodiments 2 or 5-195, antibody-drug conjugate for use according to any one of embodiments 3 or 5-195, or antibody-drug conjugate and binding agent for use according to any one of embodiments 4-195, wherein the cancer is selected from soft tissue sarcoma, vulvar cancer and gallbladder cancer, preferably wherein the target expressed on the surface of a tumour cell is TROP2.

201. The binding agent for use according to any one of embodiments 2 or 5-200, antibody-drug conjugate for use according to any of one embodiments 3 or 5-200, or antibody-drug conjugate and binding agent for use according to any one of embodiments 4-200, wherein, further comprising administering a further therapeutic agent, preferably selected from an immune checkpoint inhibitor, a chemotherapeutic agent, or a combination thereof.

202. A medical preparation or composition comprising
a. a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction, and
b. an antibody-drug conjugate comprising an antibody or fragment thereof that specifically binds to a target expressed on the surface of a tumour cell.

203. The medical preparation or composition according to embodiment 202, wherein the medical preparation or composition is a pharmaceutical composition, preferably wherein the pharmaceutical composition further comprises one or more pharmaceutically acceptable carriers, diluents and/or excipients.

204. The medical preparation or composition according to embodiment 202 or 203, wherein the binding agent and the antibody-drug conjugate are defined according to any of embodiments 5-177.

205. The medical preparation or composition according to embodiment 202 or 203, wherein the binding agent is a multispecific antibody, preferably a bispecific antibody that comprises (a) an antibody or fragment thereof that specifically binds to PD-L1 and (b) an antibody or fragment thereof that specifically binds to VEGF.

206. The medical preparation or composition according to embodiment 202 or 203, wherein the binding agent is a multispecific antibody, preferably a bispecific antibody that comprises (a) an antibody or fragment thereof that specifically binds to PD-1 and (b) an antibody or fragment thereof that specifically binds to VEGF.

207. The medical preparation or composition according to embodiment 202 or 203, wherein the binding agent is a multispecific antibody, preferably a bispecific antibody that comprises (a) an antibody or fragment thereof that specifically binds to PD-1 and (b) an antibody or fragment thereof that specifically binds to VEGFR, preferably to VEGFR2.

208. The medical preparation or composition according to any one of embodiments 202-207, wherein the medical preparation or composition further comprises a further therapeutic agent, preferably selected from an immune checkpoint inhibitor, a chemotherapeutic agent, or a combination thereof.

209. A kit-of-parts comprising
a. a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction, and
b. an antibody-drug conjugate comprising an antibody or fragment thereof that specifically binds to a target expressed on the surface of a tumour cell.

210. The kit-of-parts according to embodiment 209, wherein the binding agent and the antibody-drug conjugate are comprised in separate containers.

211. The kit-of-parts according to embodiment 209 or 210, further comprising instructions for use.

212. The kit-of-parts according to any one of embodiments 209-211, wherein the binding agent and the antibody-drug conjugate are defined according to any of embodiments 5-177.

213. The kit-of-parts according to any one of embodiments 209-212, wherein the binding agent is a multispecific antibody, preferably a bispecific antibody that comprises (a) an antibody or fragment thereof that specifically binds to PD-L1 and (b) an antibody or fragment thereof that specifically binds to VEGF.

214. The kit-of-parts according to any one of embodiments 209-212, wherein the binding agent is a multispecific antibody, preferably a bispecific antibody that comprises (a) an antibody or fragment thereof that specifically binds to PD-1 and (b) an antibody or fragment thereof that specifically binds to VEGF.

215. The kit-of-parts according to any one of embodiments 209-212, wherein the binding agent is a multispecific antibody, preferably a bispecific antibody that comprises (a) an antibody or fragment thereof that specifically binds to PD-1 and (b) an antibody or fragment thereof that specifically binds to VEGFR, preferably to VEGFR2.

216. The kit-of-parts according to any one of embodiments 209-215, wherein the kit-of-parts further comprises a further therapeutic agent, preferably selected from an immune checkpoint inhibitor, a chemotherapeutic agent, or a combination thereof.

217. The medical preparation or composition according to any one of embodiments 202-208 or the kit-of-parts according to any one of embodiments 209-216 for use as a medicament.

218. The medical preparation or composition or kit-of-parts for use according to embodiment 217, wherein the medicament is for administration to a subject.

219. The medical preparation or composition or kit-of-parts for use according to embodiment 217 or 218, wherein the use as a medicament comprises a therapeutic or prophylactic treatment of a disease or disorder.

220. The medical preparation or composition or kit-of-parts for use according to embodiment 219, wherein the therapeutic or prophylactic treatment of a disease or disorder comprises treating or preventing cancer.

221. The medical preparation or composition or kit-of-parts for use according to embodiment 220, wherein the therapeutic or prophylactic treatment of a disease or disorder further comprises administering radiotherapy, preferably local radiotherapy.

### Advantages

The combination therapy of the invention exhibits a number of advantages compared with the individual components when used alone. Without wishing to be bound by theory, the following advantages are expected to be conferred by the novel combination therapy compared with the individual components.

Firstly, the binding agent (e.g. bispecific antibody) component of the combination of the invention can specifically act as an immune amplifier. The first binding region of the binding agent (e.g. the PD-L1-targeting arm of a bispecific antibody) is capable of blocking the PD1-PDL1 interaction.

Moreover, the combination therapy of the invention may also benefit from the second binding region of the binding agent (e.g. the anti-angiogenic anti-VEGF-A arm of a bispecific antibody). Combining ADCs with such anti-angiogenic therapy, such as the binding agent (e.g. bispecific antibody) component may facilitate better tumour penetration and exposure to the ADC, resulting in more effective eradication of tumour cells.

The present inventors have surprisingly found that the effectiveness of an ADC can be increased, e.g. synergistically, by co-administration of a binding agent (e.g. bispecific antibody) which antagonizes the PD-1/PD-L1 interaction and the VEGF/VEGFR interaction. Thus, the present disclosure generally embraces the immunotherapeutic treatment of cancer in a subject comprising (1) the administration of an antibody-drug conjugate comprising an antibody or fragment thereof that specifically binds to a target expressed on the surface of a tumour cell combined with (2) the administration of a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction, thereby blocking the PD-1/PD-L1 inhibitory axis and inhibiting the VEGF/VEGFR pathway in cancer cells.

The two components of the combination therapy of the invention, namely the ADC as defined herein and the binding agent (e.g. bispecific antibody) as defined herein, are therefore considered to mutually amplify each other's effects, preferably synergistically. The ADC component can pave the way for the immunomodulatory effects of the binding agent (e.g. bispecific antibody), and the binding agent (e.g. bispecific antibody) can increase the efficacy of the ADCs in solid tumours.

### Definitions

The terms indicated for explanation of the invention and the disclosure have the following meaning, unless otherwise indicated in the description or the claims. Additional definitions are set forth throughout the detailed description.

The terms used herein may be defined as described in “A multilingual glossary of biotechnological terms: (IUPAC Recommendations)”, H. G. W. Leuenberger, B. Nagel, and H. Kolbl, Eds., Helvetica Chimica Acta, CH-4010 Basel, Switzerland, (1995).

The practice of the present disclosure may employ, unless otherwise indicated, conventional chemistry, biochemistry, cell biology, immunology, and recombinant DNA techniques which are explained in the literature in the field (cf, e.g., Organikum, Deutscher Verlag der Wissenschaften, Berlin 1990; Streitwieser/Heathcook, “Organische Chemie”, VCH, 1990; Beyer/Walter, “Lehrbuch der Organischen Chemie”, S. Hirzel Verlag Stuttgart, 1988; Carey/Sundberg, “Organische Chemie”, VCH, 1995; March, “Advanced Organic Chemistry”, John Wiley & Sons, 1985; Rompp Chemie Lexikon, Falbe/Regitz (Hrsg.), Georg Thieme Verlag Stuttgart, New York, 1989; Molecular Cloning: A Laboratory Manual, 2nd Edition, J. Sambrook et al. eds., Cold Spring Harbor Laboratory Press, Cold Spring Harbor 1989.

Terms “a” and “an” and “the” and similar reference used in the context of describing the invention (especially in the context of the claims) are to be construed to cover both the singular and the plural, unless otherwise indicated herein or clearly contradicted by context.

The terms “about” or “approximately” as used herein denotes a range of 10% of a reference value. For examples, “about 10” defines a range of 9 to 11. In general, those skilled in the art, familiar with the context, will appreciate the relevant degree of variance encompassed by “about” or “approximately” in that context.

The term “adjuvant” relates to a compound which prolongs, enhances or accelerates an immune response. Adjuvants comprise a heterogeneous group of compounds such as oil emulsions (e.g., Freund's adjuvants), mineral compounds (such as alum), bacterial products (such as Bordetella pertussis toxin), or immune-stimulating complexes. Examples of adjuvants include, without limitation, LPS, GP96, CpG oligodeoxynucleotides, growth factors, and cytokines, such as monokines, lymphokines, interleukins, chemokines. The chemokines may be IL-1, IL-2, IL-3, IL-4, IL-5, IL-6, IL-7, IL-8, IL-9, IL-10, IL-12, INFa, INF-γ, GM-CSF, LT-a. Further known adjuvants are aluminum hydroxide, Freund's adjuvant or oil such as Montanide® ISA51. Other suitable adjuvants for use in the present disclosure include lipopeptides, such as Pam3Cys, as well as lipophilic components, such as saponins, trehalose-6,6-dibehenate (TDB), monophosphoryl lipid-A (MPL), monomycoloyl glycerol (MMG), or glucopyranosyl lipid adjuvant (GLA).

The term “binding agent” in the context of the present disclosure refers to any agent capable of binding to desired antigens. In certain embodiments, the binding agent is an antibody, antibody fragment, or construct thereof. The binding agent may also comprise synthetic, modified or non-naturally occurring moieties, in particular non-peptide moieties. Such moieties may, for example, link desired antigen-binding functionalities or regions such as antibodies or antibody fragments. In one embodiment, the binding agent is a synthetic construct comprising antigen-binding CDRs or variable regions.

A binding agent of the present invention may comprise: (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction. The first binding region and the second binding region may be separate, or may overlap, partially or completely. In some embodiments, the first binding region and the second binding region are separate, e.g. the binding agent may comprise: (i) an anti-PD-1 antibody, a PD-1 binding fragment thereof, an anti-PD-L1 antibody, or a PD-L1 binding fragment thereof; and (ii) an anti-VEGF antibody, a VEGF binding fragment thereof, a protein comprising a VEGFR domain, an anti-VEGFR antibody, or a VEGF binding fragment thereof. In other embodiments, the first binding region and the second binding region overlap, partially or completely, e.g. the binding agent may be an antibody or binding fragment thereof that specifically binds to both PD-1 and VEGFR, preferably VEGFR2.

A binding region as defined herein (such as an antibody or a fragment thereof as defined herein) which “antagonizes the PD-1/PD-L1 interaction” means that the binding region is antagonistic towards the PD-1/PD-L1 signaling pathway. In particular, such binding region which antagonizes the PD-1/PD-L1 interaction totally or partially inhibits, reduces, interferes with or negatively modulates the PD-1/PD-L1 signaling pathway by binding to PD-1, PD-L1, or both. Preferably, the binding region which antagonizes the PD-1/PD-L1 interaction mediates its antagonistic activity in a manner that totally inhibits (i.e., blocks) or partially reduces the binding of PD-L1 to PD-1.

A binding region as defined herein (such as an antibody or a fragment thereof as defined herein) which “antagonizes the VEGF/VEGFR interaction” means that the binding region is antagonistic towards the VEGF/VEGFR signaling pathway. In particular, such binding region which antagonizes the VEGF/VEGFR interaction totally or partially inhibits, reduces, interferes with or negatively modulates the VEGF/VEGFR signaling pathway by binding to VEGF, VEGFR, or both. Preferably, the binding region which antagonizes the VEGF/VEGFR interaction mediates its antagonistic activity in a manner that totally inhibits (i.e., blocks) or partially reduces the binding of VEGF to VEGFR.

In the context of the present disclosure, a bispecific antibody that comprises
(a) an antibody or fragment thereof that specifically binds to PD-L1 and
(b) an antibody or fragment thereof that specifically binds to VEGF is a specific example of a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction as disclosed herein. All embodiments disclosed herein in the context of the binding agent likewise apply to the bispecific antibody.

“Antibodies” are glycoproteins belonging to the immunoglobulin superfamily. The term “full-length antibody” may refer to an immunoglobulin molecule that binds to a target molecule and contains four peptide chains: two heavy chains and two light chains which are connected to each other through disulfide bonds. Antibodies may comprise several “regions” or “domains” and the terms may be used interchangeably herein. An antibody may recognise an antigen via the fragment antigen-binding (Fab) variable region. The fragment crystallizable region (Fc region) is the tail region of an antibody that may allow antibodies to activate the immune system. The hinge region is a stretch of heavy chains linking the Fab and Fc regions. The heavy chain and light chain may each comprise a variable domain and one or more constant domains. For example, in IgG antibodies, a heavy chain comprises a variable domain (VH) and three constant domains (CH1, CH2, and CH3) and a light chain comprises a variable domain (VL) and one constant domain (CL). Examples antibodies include a human antibody, a mouse antibody, a camelid antibody, a humanized antibody, a chimeric antibody, a multispecific antibody, a monoclonal antibody, and a polyclonal antibody.

The term “antibody fragment” may refer to a fragment of an antibody, or a genetically engineered product of one of more fragments of an antibody, which fragment is involved in binding with the target molecule. Examples of antibody fragments include an antigen-binding fragment (Fab), a Fab′, a Fab′-SH, a fragment antibody (F(ab′)2), a variable region (Fv), a single chain antibody, such as a single chain variable fragment (scFv), a single-domain antibody (sdAb), a nanobody, and a VHH. The term “Antigen-binding fragment” or “Fab” refers to a region of an antibody that binds to antigens and is composed of one constant and one variable region of each of the heavy and the light chain. The term “fragment antibody” or “F(ab′)2” refers to a region of an antibody that remains following digestion of the Fc region while leaving intact some of the hinge region. The term “Fab′” refers to a fragment formed by the reduction of a F(ab′)2 fragment. The term “Fab′-SH” refers to a Fab′ fragment with a free sulfhydryl group. The term “Single chain variable fragment” or “scFv” refers to an engineered antibody consisting of a light chain variable region and a heavy chain variable region which are connected to each other.

The term “bispecific antibody” may refer to a molecule which binds to two different target molecules and which comprises or consists of one or more antibodies and/or antibody fragments. The one or more antibodies and/or antibody fragments may include any described herein, including a human antibody, a humanized antibody, a chimeric antibody, a multispecific antibody, a monoclonal antibody, a polyclonal antibody, an antigen-binding fragment (Fab), a Fab′, a Fab′-SH, a fragment antibody (F(ab′)2), a variable region (Fv), a single chain variable fragment (scFv), a single-domain antibody (sdAb), a nanobody, a VHH, and a camelid antibody. A bispecific antibody may comprise a single chain or two or more chains.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carriers include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers. In some embodiments, the pharmaceutical composition of the present disclosure includes isotonic saline.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985). Pharmaceutical carriers, excipients or diluents can be selected with regard to the intended route of administration and standard pharmaceutical practice.

“CDR” or “CDRs” means complementarity determining region(s) in an immunoglobulin variable region. The variable regions of the heavy and light chains each contain three CDRs, designated CDR1, CDR2 and CDR3. The precise boundaries of these CDRs can be defined according to various numbering systems known in the art (see e.g. Dondelinger, M., et al., 2018. Frontiers in immunology, 9, p.2278), such as the Kabat numbering system (Kabat et al., Sequences of Proteins of Immunological Interest, 5th Ed. Public Health Service, National Institutes of Health, Bethesda, Md., 1991; Kabat et al., 1992, Sequences of Proteins of Immunological Interest, DIANE Publishing: 2719), the Chothia numbering system (Chothia & Lesk (1987) J. Mol. Biol. 196:901-917; Chothia et al. (1989) Nature 342:878-883) or the IMGT numbering system (Lefranc et al., Dev. Comparat. Immunol.27:55-77, 2003; Ehrenmann F, Kaas Q, Lefranc M P. IMGT/3Dstructure-DB and IMGT/DomainGapAlign: a database and a tool for immunoglobulins or antibodies, T cell receptors, MHC, IgSF and MhcSF[J]. Nucleic acids research, 2009; 38(suppl_1):D301-D307). For a given antibody, those skilled in the art will readily identify the CDRs defined by each numbering system. Also, the correspondence between different numbering systems is well known to those skilled in the art (for example, see Lefranc et al., Dev. Comparat. Immunol. 27:55-77, 2003). “Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

Unless expressly specified otherwise, the term “comprising” is used in the context of the present disclosure to indicate that further members may optionally be present in addition to the members of the list introduced by “comprising”. It is, however, contemplated as specific embodiments of the present invention that each time the term “comprising” is used, this shall also encompass the possibility of no further members being present, i.e., for the purpose of this embodiment “comprising” can be understood as having the meaning of “consisting of”.

“Combination therapy” refers to the binding agent (e.g. bispecific antibody) disclosed herein that specifically binds to PD-L1 and VEGF in combination with an antibody-drug conjugate disclosed herein for use in a method of treating a subject with cancer. Each component of the combination therapy, i.e., the binding agent (e.g. bispecific antibody) and the antibody-drug conjugate may be administered separately. For example, the combination can be administered sequentially in this order, in reverse order, concomitantly, or combinations thereof.

The term “diluent” relates to a diluting and/or thinning agent. Moreover, the term “diluent” includes any one or more of fluid, liquid or solid suspension and/or mixing media. Examples of suitable diluents include ethanol, glycerol, and water.

Herein, the term “DNA” relates to a nucleic acid molecule which is entirely or at least substantially composed of deoxyribonucleotide residues. In preferred embodiments, the DNA contains all or a majority of deoxyribonucleotide residues. As used herein, “deoxyribonucleotide” refers to a nucleotide which lacks a hydroxyl group at the 2′-position of a β-D-ribofuranosyl group. DNA encompasses without limitation, double stranded DNA, single stranded DNA, isolated DNA such as partially purified DNA, essentially pure DNA, synthetic DNA, recombinantly produced DNA, as well as modified DNA that differs from naturally occurring DNA by the addition, deletion, substitution and/or alteration of one or more nucleotides. Such alterations may refer to addition of non-nucleotide material to internal DNA nucleotides or to the end(s) of DNA. It is also contemplated herein that nucleotides in DNA may be non-standard nucleotides, such as chemically synthesized nucleotides or ribonucleotides. For the present disclosure, these altered DNAs are considered analogs of naturally-occurring DNA. A molecule contains “a majority of deoxyribonucleotide residues” if the content of deoxyribonucleotide residues in the molecule is more than 50% (such as at least 55%, at least 60%, at least 65%, at least 70%, at least 75%, at least 80%, at least 85%, at least 90%, at least 95%, at least 96%, at least 97%, at least 98%, at least 99%), based on the total number of nucleotide residues in the molecule. The total number of nucleotide residues in a molecule is the sum of all nucleotide residues (irrespective of whether the nucleotide residues are standard (i.e., naturally occurring) nucleotide residues or analogs thereof). DNA may be recombinant DNA and may be obtained by cloning of a nucleic acid, in particular cDNA. The cDNA may be obtained by reverse transcription of RNA.

As used herein, the term “effective amount” refers to an amount of a given substance that is sufficient in quantity to produce a desired effect, including an improvement or remediation of the disease, disorder, or symptoms of the disease or condition. A therapeutically effective amount of a therapeutic agent may vary according to factors such as the disease state, age, sex, and weight of the individual, and the ability of the therapeutic agent to elicit a desired response in the individual. A therapeutically effective amount is also one in which any toxic or detrimental effects of the therapeutic agent are outweighed by the therapeutically beneficial effects. In the case that a reaction in a patient is insufficient with an initial dose, higher doses (or effectively higher doses achieved by a different, more localized route of administration) may be used. In case that unwanted side effects occur in a patient with a dose, lower doses (or effectively lower doses achieved by a different, more localized route of administration) may be used. The combination therapy described herein is to be administered to a patient in need therefore in an effective amount.

As used herein, the term “encode” or “encoding” refers to sequence information of a first molecule that guides production of a second molecule having a defined sequence of nucleotides (e.g., mRNA) or a defined sequence of amino acids. For example, a DNA molecule can encode an RNA molecule (e.g., by a transcription process that includes a DNA-dependent RNA polymerase enzyme). An RNA molecule can encode a polypeptide (e.g., by a translation process). Thus, a gene, a cDNA, or a single-stranded RNA (e.g., an mRNA) encodes a polypeptide if transcription and translation of mRNA corresponding to that gene produces the polypeptide in a cell or other biological system. In some embodiments, a coding region of a single-stranded RNA encoding a target polypeptide agent refers to a coding strand, the nucleotide sequence of which is identical to the mRNA sequence of such a target polypeptide agent. In some embodiments, a coding region of a single-stranded RNA encoding a target polypeptide agent refers to a non-coding strand of such a target polypeptide agent, which may be used as a template for transcription of a gene or cDNA. As is understood in the art, the phrase “nucleic acid encoding a peptide or protein” means that the polynucleotide, if present in the appropriate environment, for example within a cell and/or in a cell-free translation system, can direct the assembly of amino acids to produce the peptide or protein via a process of translation.

The term “epitope” refers to the part of an antigen that as used herein, refers to an agent that elicits an immune response; and/or (ii) an agent that binds to a T cell receptor (e.g., when presented by an MHC molecule) or to an antibody. For example, epitopes are the discrete, three-dimensional sites on an antigen, which are recognized by the immune system. Epitopes usually consist of chemically active surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents.

The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants.

As used herein, the term “gene” refers to a DNA sequence in a chromosome that codes for a protein. In some embodiments, a gene includes coding sequence (i.e., sequence that encodes a particular protein); in some embodiments, a gene includes non-coding sequence. In some particular embodiments, a gene may include both coding (e.g., exonic) and non-coding (e.g., intronic) sequences. In some embodiments, a gene may include one or more regulatory elements that, for example, may control or impact one or more aspects of gene expression (e.g., cell-type-specific expression, inducible expression, etc.).

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence.

The term “immune cell” means any cell of hematopoietic lineage involved in regulating an immune response against an antigen (e.g., a bacterial or viral infection or an auto-antigen). In typical embodiments, an immune cell is a leukocyte, such as a white blood cell. Immune cells include neutrophils, eosinophils, basophils, lymphocytes, and/or monocytes. Lymphocytes include T lymphocytes and B lymphocytes. Immune cells can also be dendritic cells, natural killer (NK) cells, and/or a mast cell.

As used herein, “immune checkpoint” refers to regulators of the immune system, and, in particular, co-stimulatory and inhibitory signals that regulate the amplitude and quality of T cell receptor recognition of an antigen. In certain embodiments, the immune checkpoint is an inhibitory signal. In certain embodiments, the inhibitory signal is the interaction between PD-1 and PD-L1 and/or PD-L2. The terms “checkpoint inhibitor” (CPI) and “immune checkpoint (ICP) inhibitor” are used herein synonymously. The terms refer to molecules, such as binding agents, which totally or partially reduce, inhibit, interfere with or negatively modulate one or more checkpoint proteins or that totally or partially reduce, inhibit, interfere with or negatively modulate expression of one or more checkpoint proteins, like molecules, such as binding agents, which inhibit an immune checkpoint, in particular, which inhibit the inhibitory signal of an immune checkpoint. In one embodiment, the immune checkpoint inhibitor binds to one or more checkpoint proteins. In one embodiment, the immune checkpoint inhibitor binds to one or more molecules regulating checkpoint proteins. In one embodiment, the immune checkpoint inhibitor binds to precursors of one or more checkpoint proteins e.g., on DNA- or RNA-level. Any agent that functions as a checkpoint inhibitor according to the present disclosure can be used. The term “partially” as used herein means at least 5%, 10%, 15%, 20%, 25%, 30%, 35%, 40%, 45%, 50%, 55%, 60%, 65%, 70%, 75%, 80%, 85%, 90%, 95%, 96%, 97%, 98% or 99% in the level, e.g., in the level of inhibition of a checkpoint protein.

In one embodiment, the checkpoint inhibitor can be any compound, such as any binding agent, which inhibits the inhibitory signal of an immune checkpoint. In one embodiment, the inhibitory signal is the interaction between PD-1 and PD-L1 and/or PD-L2 (such a checkpoint inhibitor which inhibits the interaction between PD-1 and PD-L1 and/or PD-L2 is also called a PD-1/PD-L1 checkpoint inhibitor herein). In one embodiment, the checkpoint inhibitor is at least one selected from the group consisting of PD-1 inhibitors, PD-L1 inhibitors, and PD-L2 inhibitors. In one embodiment, the checkpoint inhibitor may be a blocking antibody, such as a PD-1 blocking antibody, a PD-L1 blocking antibody, or a PD-L2 blocking antibody.

Examples of a PD-1 blocking antibody include pembrolizumab, nivolumab, cemiplimab, and spartalizumab. Examples of a PD-L1 blocking antibody include atezolizumab, durvalumab, and avelumab.

In one embodiment, the immune checkpoint inhibitor suitable for use in the aspects of the present disclosure is an antibody which targets PD-1 or PD-L1. An exemplary immune checkpoint inhibitor suitable for use in the aspects of the present disclosure is pembrolizumab. The “Programmed Death-1 (PD-1)” receptor refers to an immuno-inhibitory receptor belonging to the CD28 family. PD-1 (also known as CD279) is expressed predominantly on previously activated T cells in vivo, and binds to two ligands, PD-L1 (also known as B7-H1 or CD274) and PD-L2 (also known as B7-DC or CD273). The term “PD-1” as used herein includes human PD-1 (hPD-1), variants, isoforms, and species homologs of hPD-1, and analogs having at least one common epitope with hPD-1. “Programmed Death Ligand-1 (PD-L1)” is one of two cell surface glycoprotein ligands for PD-1 (the other being PD-L2) that downregulates T cell activation and cytokine secretion upon binding to PD-1. The term “PD-L1” as used herein includes human PD-L1 (hPD-L1), variants, isoforms, and species homologs of hPD-L1, such as macaque (cynomolgus monkey), African elephant, wild boar and mouse PD-L1 (cf, e.g., Genbank accession no. NP_054862.1, XP_005581836, XP_003413533, XP_005665023 and NP_068693, respectively), and analogs having at least one common epitope with hPD-L1. The term “PD-L2” as used herein includes human PD-L2 (hPD-L2), variants, isoforms, and species homologs of hPD-L2, and analogs having at least one common epitope with hPD-L2. The ligands of PD-1 (PD-L1 and PD-L2) are expressed on the surface of antigen-presenting cells, such as dendritic cells or macrophages, and other immune cells. Binding of PD-1 to PD-L1 or PD-L2 results in downregulation of T cell activation. Cancer cells expressing PD-L1 and/or PD-L2 are able to switch off T cells expressing PD-1 what results in suppression of the anticancer immune response. The interaction between PD-1 and its ligands results in a decrease in tumor infiltrating lymphocytes, a decrease in T cell receptor mediated proliferation, and immune evasion by the cancerous cells. Immune suppression can be reversed by inhibiting the local interaction of PD-1 with PD-L1, and the effect is additive when the interaction of PD-1 with PD-L2 is blocked as well.

Many of the immune checkpoints are regulated by interactions between specific receptor and ligand pairs, such as those described above. Thus, immune checkpoint proteins mediate immune checkpoint signaling. For example, checkpoint proteins directly or indirectly regulate T cell activation, T cell proliferation and/or T cell function. Cancer cells often exploit these checkpoint pathways to protect themselves from being attacked by the immune system. Hence, the function of checkpoint proteins is typically the regulation of T cell activation, T cell proliferation and/or T cell function. Immune checkpoint proteins thus regulate and maintain self-tolerance and the duration and amplitude of physiological immune responses. Many of the immune checkpoint proteins belong to the B7:CD28 family or to the tumor necrosis factor receptor (TNFR) super family and, by binding to specific ligands, activate signaling molecules that are recruited to the cytoplasmic domain (Suzuki et al., 2016, Jap J Clin Onc, 46:191-203).

The term “VEGF” as used herein refers to vascular endothelial growth factor (VEGF) which is a member of the platelet-derived growth factor family of cystine-knot growth factors, i.e., a group of signal proteins involved in vasculogenesis and angiogenesis. Upon binding to tyrosine kinase receptors (VEGF receptors (VEGFRs)), the VEGFR dimerizes and becomes activated through transphosphorylation. Overexpression of VEGF can cause vascular diseases. Drugs which target VEGF (e.g., aflibercept, bevacizumab, ranibizumab, and pegaptanib) can inhibit VEGF and, thus, slow such diseases. In one embodiment, VEGF is human VEGF, in particular human VEGF comprising the amino acid sequence of SEQ ID NO: 176. In some embodiments, VEGF is human VEGF-A, in particular human VEGF-A comprising the amino acid sequence of SEQ ID NO: 177.

The term “VEGFR” as used herein refers to a VEGF receptor, which is tyrosine kinase receptor. Upon binding of a VEGF to the VEGFR, the VEGFR dimerizes and becomes activated through transphosphorylation.

The term “VEGFR2” refers to Vascular Endothelial Growth Factor Receptor 2 (also known as KDR or FLK1) and variants thereof that retain at least part of the biological activity of VEGFR2. As used herein, VEGFR2 may include all species of native sequence VEGFR2, including human, rat, mouse and chicken. The term “VEGFR2” may be used to include variants, isoforms and species homologs of human VEGFR2. In some embodiments, “VEGFR2” refers to the wild-type human form of VEGFR2. Antibodies of the invention may cross-react with VEGFR2 from species other than human, in particular VEGFR2 from rhesus monkey (Macaca mulatta). Examples of human and rhesus VEGFR2 amino acid sequences are provided in Table 1. In certain embodiments, the antibodies may be completely specific for human VEGFR2 and may not exhibit non-human cross-reactivity. In one embodiment, VEGFR2 is human VEGFR2, in particular human VEGFR2 comprising the amino acid sequence of SEQ ID NO: 344. In some embodiments, VEGFR2 is monkey VEGFR2, in particular Macaca VEGFR2 comprising the amino acid sequence of SEQ ID NO: 345.

“Isolated” means altered or removed from the natural state. For example, a nucleic acid or a peptide naturally present in a living animal is not “isolated”, but the same nucleic acid or peptide partially or completely separated from the coexisting materials of its natural state is “isolated”. An isolated nucleic acid or protein can exist in substantially purified form, or can exist in a non-native environment such as, for example, a host cell. In a preferred embodiment, the combination partner used in the present disclosure is in substantially purified form.

The term “monoclonal antibody” refers to an antibody obtained from a substantially homogeneous population of antibodies. The individual antibodies composing the population may be identical except for possible naturally occurring mutations, which may be present in minor amounts. Monoclonal antibodies are highly specific and target a single antigenic epitope. In contrast, polyclonal antibody preparations typically include a large number of antibodies which are specific for different epitopes.

The term “multispecific antibody” refers to antibodies which recognise two or more epitopes located on the same or distinct targets. Formats of multispecific antibodies can be divided into two broad categories: IgG-like antibody formats, with an Fc domain, and non-IgG-like antibody formats, without an Fc domain (see e.g. Elshiaty, M., et al., 2021. International journal of molecular sciences, 22(11), p.5632). Examples of multispecific antibodies include bispecific and trispecific antibodies.

The term “pharmaceutical composition” relates to a composition comprising a therapeutically effective agent, preferably together with pharmaceutically acceptable carriers, diluents and/or excipients. Said pharmaceutical composition is useful for treating, preventing, or reducing the severity of a disease by administration of said pharmaceutical composition to a subject.

The term “pharmaceutically acceptable carrier” or “pharmaceutically acceptable excipient” means solvents, dispersion media, coatings, antibacterial agents and antifungal agents, isotonic agents, and absorption delaying agents, and the like, that are compatible with pharmaceutical administration. The use of such media and agents for pharmaceutically active substances is well known in the art. In certain embodiments, the pharmaceutically acceptable carrier or excipient is not naturally occurring.

The term “plurality” refers to the state of being plural.

The terms “polynucleotide” and “nucleic acid” can be used interchangeably herein to refer to polymers of nucleotides. The term “polynucleotide” comprises deoxyribonucleic acid (DNA), ribonucleic acid (RNA), combinations thereof, and modified forms thereof. The term comprises genomic DNA, cDNA, mRNA, recombinantly produced and chemically synthesized molecules. In some embodiments, a polynucleotide is DNA. In some embodiments, a polynucleotide is RNA. In some embodiments, a polynucleotide is a mixture of DNA and RNA. A polynucleotide may be present as a single-stranded or double-stranded and linear or covalently circularly closed molecule. A polynucleotide can be isolated. The term “isolated polynucleotide” means, according to the present disclosure, that the polynucleotide (i) was amplified in vitro, for example via polymerase chain reaction (PCR) for DNA or in vitro transcription (using, e.g., an RNA polymerase) for RNA, (ii) was produced recombinantly by cloning, (iii) was purified, for example, by cleavage and separation by gel electrophoresis, or (iv) was synthesized, for example, by chemical synthesis. The terms “polypeptide,” “peptide,” and “protein” are used interchangeably herein to refer to polymers of amino acids.

“Kabat,” as used herein, means an immunoglobulin alignment and numbering system pioneered by Elvin A. Kabat ((1991) Sequences of Proteins of Immunological Interest, 5th Ed. Public Health Service, National Institutes of Health, Bethesda, Md.).

The term “recombinant” when used in the context of a polynucleotide means a polynucleotide having nucleotide sequences that are not naturally joined together and can be made by artificially combining two otherwise separated segments of sequence. This artificial combination is often accomplished by chemical synthesis or, more commonly, by the artificial manipulation of isolated segments of nucleic acids, for example, by genetic engineering techniques. Recombinant polynucleotides include vectors comprising an amplified or assembled polynucleotide, which can be used to transform or transfect a suitable host cell. A host cell that comprises the recombinant polynucleotide is referred to as a “recombinant host cell.” The polynucleotide is then expressed in the recombinant host cell to produce a “recombinant polypeptide.” A recombinant polynucleotide can also comprise a non-coding function.

A “single domain antibody” (sdAb) is an antibody composed of a single variable domain (e.g., heavy chain variable region) composed of antibody fragments. Typically, a single domain antibody, domain antibody or nanobody consists of 4 framework regions and 3 complementarity determining regions, the 4 framework regions are respectively FR1-FR4, and the 3 complementarity determining regions are respectively CDR1-CDR3. In certain embodiments, the single domain antibody of the present application may have a structure of FR1-CDR1-FR2-CDR2-FR3-CDR3-FR4. These antibodies do not require light chain variable regions to bind antigens with high affinity and specificity. Examples of single-domain antibodies include, but are not limited to, VHH fragments, and VNAR fragments. Compared with antibodies composed of heavy chain and light chain, single domain antibodies have high solubility, high stability to heat, pH, protease and other deforming agents, and only need single-chain expression to facilitate large-scale production. As used herein, the term “framework region” or “FR” residues refers to those amino acid residues in an antibody variable region other than the CDR residues as defined above.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g., without producing undesired results such as false positives. Antibodies, or binding fragments thereof, useful in the present invention will bind to the target protein with an affinity that is at least two-fold greater, preferably at least ten times greater, more preferably at least 20-times greater, and most preferably at least 100-times greater than the affinity with non-target proteins. As used herein, an antibody is said to bind specifically to a polypeptide comprising a given amino acid sequence, e.g., the amino acid sequence of a mature human PD-L1 molecule, if it binds to polypeptides comprising that sequence but does not bind to proteins lacking that sequence.

As used herein, a “subject” is a human of either gender (a male or a female). The subject may be of any age. In one embodiment, the subject is female. In another embodiment, the subject is male. In some embodiments, the subject is a subject having cancer, in particular a female subject having cancer and/or a male subject having cancer. In some embodiments, the subject is naïve to PD-1/PD-L1 targeted treatment, VEGF targeted treatment or both. In some embodiments, the subject is naïve to PD-1/PD-L1 targeted treatment and VEGF targeted treatment.

The term “treating” or “treatment” when used in the context of a disease or disease condition means ameliorating, improving or remedying a disease, disorder, or symptom of a disease or condition associated with the disease, or can mean completely or partially stopping, on a molecular level, the biochemical basis of the disease, etc. It describes an act that leads to the elimination, reduction, alleviation, reversal, or prevention or delay of onset or recurrence of any symptom of a disease.

The term “triple-negative breast cancer” as used herein is used in the usually sense and refers to the cancer class that tests negative for estrogen receptors and progesterone receptors expression, and HER2 overexpression or gene amplification. TNBC is human epidermal growth factor receptor 2 (HER2) negative and has <1% expression of estrogen receptors (ER) and progesterone receptors (PR) by immunostaining. It is a biologically aggressive tumour, characterized by moderate/high grade and highly proliferative cancer cells, which, together with limited treatment options leads to the poorest prognosis among the breast cancer subtypes.

“Tumour” as it applies to a subject diagnosed with, or suspected of having, a cancer refers to a malignant or potentially malignant neoplasm or tissue mass of any size and includes primary tumours and secondary neoplasms. A solid tumour is an abnormal growth or mass of tissue that usually does not contain cysts or liquid areas. Different types of solid tumours are named for the type of cells that form them. Examples of solid tumours are sarcomas, carcinomas, and lymphomas. Leukemias (cancers of the blood) generally do not form solid tumours (National Cancer Institute, Dictionary of Cancer Terms).

“Variable regions” as used herein means a segment of an antibody which contains three CDRs, designated CDR1, CDR2 and CDR3. A “variable region” of an antibody refers to the variable region of the antibody light chain or the variable region of the antibody heavy chain, either alone or in combination. The variable region of the heavy chain may be referred to as “VH.” The variable region of the light chain may be referred to as “VL.” Typically, the variable regions of both the heavy and light chains comprise three hypervariable regions, the CDRs, which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3, and FR4.

The term “alkyl” refers to a monoradical of a saturated straight or branched hydrocarbon. Preferably, the alkyl group comprises from 1 to 40, i.e., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40, carbon atoms, such as 1 to 30, such as 1 to 20 carbon atoms, such as 1 to 12 carbon atoms, such as 1 to 10 carbon atoms, such as 1 to 8 carbon atoms, such as 1 to 6 or 1 to 4 carbon atoms. Exemplary alkyl groups include methyl, ethyl, propyl, iso-propyl (also called 2-propyl or 1 methylethyl), butyl, iso-butyl, tert-butyl, n-pentyl, iso-pentyl, sec-pentyl, neo-pentyl, 1,2-dimethylpropyl, iso-amyl, n-hexyl, iso-hexyl, sec-hexyl, n-heptyl, iso-heptyl, n-octyl, 2-ethyl-hexyl, n-nonyl, n-decyl, n-undecyl, n-dodecyl, n-undecyl, n-dodecyl, n-tridecyl, n-tetradecyl, n-pentadecyl, n-hexadecyl, n-heptadecyl, n-octadecyl, n-nonadecyl, n-icosyl, n-triacontyl, n-tetracontyl, and the like. A “substituted alkyl” means that one or more (such as 1 to the maximum number of hydrogen atoms bound to an alkyl group, e.g., 1, 2, 3, 4, 5, 6, 7, 8, 9, or up to 10, such as between 1 to 5, 1 to 4, or 1 to 3, or 1 or 2) hydrogen atoms of the alkyl group are replaced with a substituent other than hydrogen (when more than one hydrogen atom is replaced the substituents may be the same or different). In one embodiment, the alkyl is substituted with one or more, such as 1, 2 or 3, such as 1 or 2, such as 1 substituents selected from List A. Examples of a substituted alkyl include chloromethyl, dichloromethyl, fluoromethyl, and difluoromethyl.

The term “alkylene” refers to a diradical of a saturated straight or branched hydrocarbon. Preferably, the alkylene group comprises from 1 to 40, i.e., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40, carbon atoms, such as 1 to 30, such as 1 to 20 carbon atoms, such as 1 to 12 carbon atoms, such as 1 to 10 carbon atoms, such as 1 to 8 carbon atoms, such as 1 to 6 or 1 to 4 carbon atoms. Exemplary alkylene groups include methylene, ethylene (i.e., 1,1-ethylene, 1,2-ethylene), propylene (i.e., 1,1-propylene, 1,2-propylene (—CH(CH3)CH2-), 2,2-propylene (—C(CH3)2-), and 1,3-propylene), the butylene isomers (e.g., 1,1-butylene, 1,2-butylene, 2,2-butylene, 1,3-butylene, 2,3-butylene (cis or trans or a mixture thereof), 1,4-butylene, 1,1-iso-butylene, 1,2-iso-butylene, and 1,3-iso-butylene), the pentylene isomers (e.g., 1,1-pentylene, 1,2-pentylene, 1,3-pentylene, 1,4-pentylene, 1,5-pentylene, 1,1-iso-pentylene, 1,1-sec-pentyl, 1,1-neo-pentyl), the hexylene isomers (e.g., 1,1-hexylene, 1,2-hexylene, 1,3-hexylene, 1,4-hexylene, 1,5-hexylene, 1,6-hexylene, and 1,1-isohexylene), the heptylene isomers (e.g., 1,1-heptylene, 1,2-heptylene, 1,3-heptylene, 1,4-heptylene, 1,5-heptylene, 1,6-heptylene, 1,7-heptylene, and 1,1-isoheptylene), the octylene isomers (e.g., 1,1-octylene, 1,2-octylene, 1,3-octylene, 1,4-octylene, 1,5-octylene, 1,6-octylene, 1,7-octylene, 1,8-octylene, and 1,1-isooctylene), and the like. The straight alkylene moieties having at least 3 carbon atoms and a free valence at each end can also be designated as a multiple of methylene (e.g., 1,4-butylene can also be called tetramethylene). Generally, instead of using the ending “ylene” for alkylene moieties as specified above, one can also use the ending “diyl” (e.g., 1,2-butylene can also be called butan-1,2-diyl). A “substituted alkylene” means that one or more (such as 1 to the maximum number of hydrogen atoms bound to an alkylene group, e.g., 1, 2, 3, 4, 5, 6, 7, 8, 9, or up to 10, such as between 1 to 5, 1 to 4, or 1 to 3, or 1 or 2) hydrogen atoms of the alkylene group are replaced with a substituent other than hydrogen (when more than one hydrogen atom is replaced the substituent may be the same or different). In one embodiment, the alkylene is substituted with one or more, such as 1, 2 or 3, such as 1 or 2, such as 1 substituents selected from List A.

The term “alkenyl” refers to a monoradical of an unsaturated straight or branched hydrocarbon having at least one carbon-carbon double bond. Generally, the maximal number of carbon-carbon double bonds in the alkenyl group can be equal to the integer which is calculated by dividing the number of carbon atoms in the alkenyl group by 2 and, if the number of carbon atoms in the alkenyl group is uneven, rounding the result of the division down to the next integer. For example, for an alkenyl group having 9 carbon atoms, the maximum number of carbon-carbon double bonds is 4. Preferably, the alkenyl group has 1 to 6 (such as 1 to 4), i.e., 1, 2, 3, 4, 5, or 6, carbon-carbon double bonds. Preferably, the alkenyl group comprises from 2 to 40 carbon atoms, such as 2 to 30 carbon atoms, such as 2 to 20 carbon atoms, such as 2 to 12 carbon atoms, such as 2 to 10 carbon atoms, such as 2 to 8 carbon atoms, such as 2 to 6 carbon atoms or 2 to 4 carbon atoms. Thus, in a preferred embodiment, the alkenyl group comprises from 2 to 40, such as 2 to 30, such as 2 to 20, such as 2 to 12, such as 2 to 10 carbon atoms and 1, 2, 3, 4, 5, or 6 (e.g., 1, 2, 3, 4, or 5) carbon-carbon double bonds, such as comprises 2 to 8 carbon atoms and 1, 2, 3, or 4 carbon-carbon double bonds, such as 2 to 6 carbon atoms and 1, 2, or 3 carbon-carbon double bonds or 2 to 4 carbon atoms and 1 or 2 carbon-carbon double bonds. The carbon-carbon double bond(s) may be in cis (Z) or trans (E) configuration. Exemplary alkenyl groups include vinyl, 1-propenyl, 2-propenyl (i.e., allyl), 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, 11-dodecenyl, and the like. A “substituted alkenyl” means that one or more (such as 1 to the maximum number of hydrogen atoms bound to an alkenyl group, e.g., 1, 2, 3, 4, 5, 6, 7, 8, 9, or up to 10, such as between 1 to 5, 1 to 4, or 1 to 3, or 1 or 2) hydrogen atoms of the alkenyl group are replaced with a substituent other than hydrogen (when more than one hydrogen atom is replaced the substituents may be the same or different). In one embodiment, the alkenyl is substituted with one or more, such as 1, 2 or 3, such as 1 or 2, such as 1 substituents selected from List A.

The term “alkynyl” refers to a linear or branched monovalent hydrocarbon moiety having at least one carbon-carbon triple bond in which the total carbon atoms may be six to forty, such as six to thirty, typically six to twenty, such as six to eighteen. Alkynyl groups can optionally have one or more carbon-carbon triple bonds. Generally, the maximal number of carbon-carbon triple bonds in the alkynyl group can be equal to the integer which is calculated by dividing the number of carbon atoms in the alkynyl group by 2 and, if the number of carbon atoms in the alkynyl group is uneven, rounding the result of the division down to the next integer. For example, for an alkynyl group having 9 carbon atoms, the maximum number of carbon-carbon triple bonds is 4. Preferably, the alkynyl group has 1 to 6 (such as 1 to 4), i.e., 1, 2, 3, 4, 5, or 6, more preferably 1 or 2 carbon-carbon triple bonds. A “substituted alkynyl” means that one or more (such as 1 to the maximum number of hydrogen atoms bound to an alkynyl group, e.g., 1, 2, 3, 4, 5, 6, 7, 8, 9, or up to 10, such as between 1 to 5, 1 to 4, or 1 to 3, or 1 or 2) hydrogen atoms of the alkynyl group are replaced with a substituent other than hydrogen (when more than one hydrogen atom is replaced the substituents may be the same or different). In one embodiment, the alkynyl is substituted with one or more, such as 1, 2 or 3, such as 1 or 2, such as 1 substituents selected from List A.

The term “alkoxy” means “alkyl-O—” where alkyl is as defined above, either in its broadest aspect or any of the above preferred aspects.

The term “alkylthio” means “alkyl-S—” where alkyl is as defined above, either in its broadest aspect or any of the above preferred aspects.

The term “alkylamino” means “alkyl-NR—” where alkyl is as defined above, either in its broadest aspect or any of the above preferred aspects, and R is H or alkyl (as defined above, either in its broadest aspect or any of the above preferred aspects).

“Mercapto” means —SH.

“Hydroxy” means —OH.

“Nitro” means —NO2.

“Cyano” means —CN.

“Amido” means —CONR2, wherein each R is independently H or alkyl (as defined above, either in its broadest aspect or any of the above preferred aspects).

The terms “cycloalkyl” and “cycloalkenyl” represents cyclic non-aromatic versions of “alkyl” and “alkenyl” with preferably 3 to 40, such as 3 to 30, such as 3 to 20, such as 3 to 14 carbon atoms, such as 3 to 12 or 3 to 10 carbon atoms, i.e., 3, 4, 5, 6, 7, 8, 9, 10, 11, 12, 13, or 14 carbon atoms (such as 3, 4, 5, 6, 7, 8, 9, or 10 carbon atoms), more preferably 3 to 7 carbon atoms. Exemplary cycloalkyl groups include cyclopropyl, cyclobutyl, cyclopentyl, cyclohexyl, cycloheptyl, cyclooctyl, cyclononyl, cyclodecyl, and adamantyl. Exemplary cycloalkenyl groups include cyclopropenyl, cyclobutenyl, cyclopentenyl, cyclohexenyl, cycloheptenyl, cyclooctenyl, cyclononenyl, and cyclodecenyl. The cycloalkyl or cycloalkenyl group may consist of one ring (monocyclic), two rings (bicyclic), or more than two rings (polycyclic). A “substituted cycloalkyl” means that one or more (such as 1 to the maximum number of hydrogen atoms bound to a cycloalkyl group, e.g., 1, 2, 3, 4, 5, 6, 7, 8, 9, or up to 10, such as between 1 to 5, 1 to 4, or 1 to 3, or 1 or 2) hydrogen atoms of the cycloalkyl group are replaced with a substituent other than hydrogen (when more than one hydrogen atom is replaced the substituents may be the same or different). In one embodiment, the cycloalkyl or cycloalkenyl is substituted with one or more, such as 1, 2 or 3, such as 1 or 2, such as 1 substituents selected from List A.

The term “cycloalkoxy” means “cycloalkyl-O—” where cycloalkyl is as defined above, either in its broadest aspect or any of the above preferred aspects.

The term “cycloalkylthio” means “cycloalkyl-S—” where cycloalkyl is as defined above, either in its broadest aspect or any of the above preferred aspects.

The term “aryl” refers to a monoradical of an aromatic cyclic hydrocarbon. Preferably, the aryl group contains 3 to 14 (e.g., 5, 6, 7, 8, 9, or 10, such as 5, 6, or 10) carbon atoms which can be arranged in one ring (e.g., phenyl) or two or more condensed rings (e.g., naphthyl). Exemplary aryl groups include cyclopropenylium, cyclopentadienyl, phenyl, indenyl, naphthyl, azulenyl, fluorenyl, anthryl, and phenanthryl. Preferably, “aryl” refers to a monocyclic ring containing 6 carbon atoms or an aromatic bicyclic ring system containing 10 carbon atoms. Preferred examples are phenyl and naphthyl. Aryl does not encompass fullerenes. A “substituted aryl” means that one or more (such as 1 to the maximum number of hydrogen atoms bound to an aryl group, e.g., 1, 2, 3, 4, 5, 6, 7, 8, 9, 5 or up to 10, such as between 1 to 5, 1 to 4, or 1 to 3, or 1 or 2) hydrogen atoms of the aryl group are replaced with a substituent other than hydrogen (when more than one hydrogen atom is replaced the substituents may be the same or different). In one embodiment, the aryl is substituted with one or more, such as 1, 2 or 3, such as 1 or 2, such as 1 substituents selected from List A. Examples of a substituted aryl include biphenyl, 2-fluorophenyl, 2-chloro-6-methylphenyl, anilinyl, 4-hydroxyphenyl, and methoxyphenyl (i.e., 2-, 3-, or 4-methoxyphenyl).

The term “heteroaryl” or “heteroaromatic ring” means an aryl group as defined above in which one or more carbon atoms in the aryl group are replaced by heteroatoms of O, S, or N. Preferably, heteroaryl refers to a five or six-membered aromatic monocyclic ring wherein 1, 2, or 3 carbon atoms are replaced by the same or different heteroatoms of O, N, or S. Alternatively, it means an aromatic bicyclic or tricyclic ring system wherein 1, 2, 3, 4, or 5 carbon atoms are replaced with the same or different heteroatoms of O, N, or S. Preferably, in each ring of the heteroaryl group the maximum number of O atoms is 1, the maximum number of S atoms is 1, and the maximum total number of O and S atoms is 2. Exemplary heteroaryl groups include furanyl, thienyl, oxazolyl, isoxazolyl, oxadiazolyl, pyrrolyl, imidazolyl, pyrazolyl, triazolyl, tetrazolyl, thiazolyl, isothiazolyl, thiadiazolyl, pyridyl, pyrimidinyl, pyrazinyl, triazinyl, benzofuranyl, indolyl, isoindolyl, benzothienyl, 1H-indazolyl, benzimidazolyl, benzoxazolyl, indoxazinyl, benzisoxazolyl, benzothiazolyl, benzisothiazolyl, benzotriazolyl, quinolinyl, isoquinolinyl, benzodiazinyl, quinoxalinyl, quinazolinyl, benzotriazinyl, pyridazinyl, phenoxazinyl, thiazolopyridinyl, pyrrolothiazolyl, phenothiazinyl, isobenzofuranyl, chromenyl, xanthenyl, pyrrolizinyl, indolizinyl, indazolyl, purinyl, quinolizinyl, phthalazinyl, naphthyridinyl, cinnolinyl, pteridinyl, carbazolyl, phenanthridinyl, acridinyl, perimidinyl, phenanthrolinyl, and phenazinyl. Exemplary 5- or 6-memered heteroaryl groups include furanyl, thienyl, oxazolyl, isoxazolyl, oxadiazolyl, pyrrolyl, imidazolyl (e.g., 2-imidazolyl), pyrazolyl, triazolyl, tetrazolyl, thiazolyl, isothiazolyl, thiadiazolyl, pyridyl (e.g., 4-pyridyl), pyrimidinyl, pyrazinyl, triazinyl, and pyridazinyl. A “substituted heteroaryl” means that one or more (such as 1 to the maximum number of hydrogen atoms bound to a heteroaryl group, e.g., 1, 2, 3, 4, 5, 6, 7, 8, 9, or up to 10, such as between 1 to 5, 1 to 4, or 1 to 3, or 1 or 2) hydrogen atoms of the heteroaryl group are replaced with a substituent other than hydrogen (when more than one hydrogen atom is replaced the substituents may be the same or different). In one embodiment, the heteroaryl is substituted with one or more, such as 1, 2 or 3, such as 1 or 2, such as 1 substituents selected from List A.

The term “heterocyclyl” or “heterocyclic ring” or “heterocycloalkyl” means a cycloalkyl group as defined above in which from 1, 2, 3, or 4 carbon atoms in the cycloalkyl group are replaced by heteroatoms of oxygen, nitrogen, silicon, selenium, phosphorus, or sulfur, preferably O, S, or N. A heterocyclyl group has preferably 1 or 2 rings containing from 3 to 10, such as 3, 4, 5, 6, or 7, ring atoms. Preferably, in each ring of the heterocyclyl group the maximum number of O atoms is 1, the 5 maximum number of S atoms is 1, and the maximum total number of O and S atoms is 2. The term “heterocyclyl” is also meant to encompass partially or completely hydrogenated forms (such as dihydro, tetrahydro or perhydro forms) of the above-mentioned heteroaryl groups. Exemplary heterocyclyl groups include morpholinyl, pyrrolidinyl, imidazolidinyl, pyrazolidinyl, piperidinyl (also called piperidyl), piperazinyl, di- and tetrahydrofuranyl, di- and tetrahydrothienyl, di- and tetrahydropyranyl, urotropinyl, lactones, lactams, cyclic imides, and cyclic anhydrides. A “substituted heterocyclyl” means that one or more (such as 1 to the maximum number of hydrogen atoms bound to a heterocyclyl group, e.g., 1, 2, 3, 4, 5, 6, 7, 8, 9, or up to 10, such as between 1 to 5, 1 to 4, or 1 to 3, or 1 or 2) hydrogen atoms of the heterocyclyl group are replaced with a substituent other than hydrogen (when more than one hydrogen atom is replaced the substituents may be the same or different). In one embodiment, the heterocyclyl is substituted with one or more, such as 1, 2 or 3, such as 1 or 2, such as 1 substituents selected from List A.

The term “heterocycloalkoxy” means “heterocycloalkyl-O—” where heterocycloalkyl is as defined above, either in its broadest aspect or any of the above preferred aspects.

The term “heterocycloalkylthio” means “heterocycloalkyl-S—” where heterocycloalkyl is as defined above, either in its broadest aspect or any of the above preferred aspects.

The term “arylene” generally refers to a group having two residues derived by removal of two hydrogen atoms from the same carbon atom or two different carbon atoms of the aromatic ring. The term “aromatic ring” may refer to a 6-14 membered all-carbon monocyclic ring or fused polycyclic ring (i.e., rings which share adjacent pairs of carbon atoms) having a conjugated π-electron system, and it may be 6-10 membered, such as benzene and naphthalene. The aromatic ring can be fused to a heteroaryl, heterocyclyl or cycloalkyl ring, wherein the ring connected to the parent moiety is the aryl ring. Aryl may be substituted or unsubstituted, and when it is substituted, the substituent is preferably one or more of the following groups independently selected from the group consisting of alkyl, alkenyl, alkynyl, alkoxy, alkylthio, alkylamino, halogen, mercapto, hydroxy, nitro, cyano, cycloalkyl, heterocycloalkyl, aryl, heteroaryl, cycloalkoxy, heterocycloalkoxy, cycloalkylthio and heterocycloalkylthio.

In the present application, the term “heteroarylene” generally refers to a group having two residues derived by removal of two hydrogen atoms from the same carbon atom or two different carbon atoms of the heteroaromatic ring. The term “heteroaromatic ring” refers to a heteroaromatic system comprising 1 to 4 heteroatoms and 5 to 14 ring atoms, wherein the heteroatoms may be selected from the group consisting of oxygen, sulfur and nitrogen. Heteroaryl may be 5-10 membered and may be 5- or 6-membered, such as furanyl, thienyl, pyridinyl, pyrrolyl, N-alkylpyrrolyl, pyrimidinyl, pyrazinyl, imidazolyl and tetrazolyl. The heteroaromatic ring can be fused to an aryl, heterocyclyl or cycloalkyl ring, wherein the ring connected to the parent moiety is the heteroaromatic ring. Heteroarylene may be optionally substituted or unsubstituted, and when it is substituted, the substituent is preferably one or more of the following groups independently selected from the group consisting of alkyl, alkenyl, alkynyl, alkoxy, alkylthio, alkylamino, halogen, mercapto, hydroxy, nitro, cyano, cycloalkyl, heterocycloalkyl, aryl, heteroaryl, cycloalkoxy, heterocycloalkoxy, cycloalkylthio and heterocycloalkylthio.

In the present application, the term “heterocyclylene” generally refers to a 3-7 membered monocyclic structure, a fused 7-10 membered bicyclic heterocyclic structure or a bridged 6-10 membered bicyclic heterocyclic structure that is stable and non-aromatic. These cyclic structures may be saturated or partially saturated, and contain one or more heteroatoms in addition to carbon atoms, wherein the heteroatoms may be selected from the group consisting of oxygen, sulfur and nitrogen. For example, they contain 1 to 4 heteroatoms as defined above. When used to refer to atoms on a heterocyclic cyclic structure, the term “nitrogen” may include nitrogen that undergoes a substitution reaction. Heterocyclylene may be substituted or unsubstituted.

In the present application, the term “carbocyclylene” generally refers to a group having two residues derived by removal of two hydrogen atoms from the same carbon atom or two different carbon atoms of the carbon ring. The term “carbon ring” generally refers to a saturated or partially unsaturated monocyclic or polycyclic cyclic hydrocarbon, and it contains 3 to 20 carbon atoms, may contain 3 to 12 carbon atoms, may contain 3 to 10 carbon atoms, and may contain 3 to 8 carbon atoms. Non-limiting examples of monocyclic carbon ring include cyclopropane, cyclobutane, cyclopentane, cyclopentene, cyclohexane, cyclohexene, cyclohexadiene, cycloheptane, cycloheptatriene, cyclooctane, and the like; polycyclic carbon ring may include spiro, fused and bridged carbon rings. Carbocyclylene may be substituted or unsubstituted.

The term “partially unsaturated” generally means that the cyclic structure contains at least one double or triple bond between the ring molecules. The term “partially unsaturated” encompasses cyclic structures having multiple sites of unsaturation, but is not intended to include aromatic or heteroaromatic rings defined herein. The term “unsaturated” means that the moiety has one or more degrees of unsaturation.

The term “halogen” generally refers to fluorine, chlorine, bromine or iodine, and it may be, for example, fluorine or chlorine.

In the present application, the term “aliphatic group” generally refers to a linear hydrocarbon, branched hydrocarbon or cyclic hydrocarbon having 1 to 12 carbon atoms, and the hydrocarbon may be either a fully saturated hydrocarbon or a hydrocarbon with one or more unsaturated units, but the unsaturated units are not aromatic groups. For example, suitable aliphatic groups may include substituted or unsubstituted linear, branched or cyclic alkyl, alkenyl, alkynyl and mixtures thereof, such as (cycloalkyl)alkyl, (cycloalkenyl)alkyl or (cycloalkyl)alkenyl. For example, aliphatic groups have 1 to 12, 1 to 8, 1 to 6, 1 to 4 or 1 to 3 carbon atoms.

In the present application, the term “optional” or “optionally” generally means that the event or circumstance subsequently described may, but not necessarily, occur, and that the description includes instances where the event or circumstance occurs or does not occur. For example, “heterocyclyl group optionally substituted with alkyl” means that alkyl may be, but not necessarily, present, and that the description may include instances where the heterocyclyl group is or is not substituted with alkyl.

In the present application, the term “substituted” generally means that one or more hydrogen atoms in the group, for example, up to 5 (e.g., 1 to 3) hydrogen atoms, are each independently substituted with a corresponding number of substituents. A substituent is only in its possible chemical position, and those skilled in the art will be able to determine (by experiments or theories) possible or impossible substitution without undue efforts. For example, it may be unstable when amino or hydroxy having a free hydrogen is bound to a carbon atom having an unsaturated (such as olefin) bond.

In the present application, the term “0 or more (e.g., 0 or at least 1, 0 or 1, or 0) methylene units are replaced “generally means that when the structure comprises one or more methylene units, the one or more methylene units may be unsubstituted or replaced by one or more groups that are not methylene (e.g., —NHC(O)—, —C(O)NH—, —C(O)—, —OC(O)—, —C(O)O—, —NH—, —O—, —S—, —SO—, —SO2-, —PH—, —P(═O)H—, —NHSO2-, —SO2NH—, —C(═S)—, —C(═NH)—, —N═N—, —C═N—, —N═C— or —C(═N2)-).

One or more hydrogen atoms in the group, for example, up to 5 (e.g., 1 to 3) hydrogen atoms, are each independently substituted with a corresponding number of substituents. A substituent is only in its possible chemical position, and those skilled in the art will be able to determine (by experiments or theories) possible or impossible substitution without undue efforts. For example, it may be unstable when amino or hydroxy having a free hydrogen is bound to a carbon atom having an unsaturated (such as olefin) bond.

Preferably substituents are selected from “List A”, consisting of C1-6 alkyl, C2-6 alkenyl, C2-6 alkynyl, 6- to 14-membered (such as 6- to 10-membered) aryl, 3- to 14-membered (such as 5- or 6-membered) heteroaryl, 3- to 14-membered (such as 3- to 7-membered) cycloalkyl, 3- to 14-membered (such as 3- to 7-membered) heterocyclyl, halogen, —CN, azido, —NO2, —OR′, —N(R′)2, —S(O)0-2R′, —S(O)1-2OR′, —OS(O)1-2R′, —OS(O)1-2OR′, —S(O)1-2N(R′)2, —OS(O)1-2N(R′)2, —N(R′)S(O)1-2R′, —N(R′)S(O)1-2OR′, —C(═X1)R′, —C(═X1)X1R′, —X1C(═X1)R′, and —X1C(═X1)X1R′, wherein X1 is independently selected from O, S, NH and N(CH3); and each R′ is independently selected from the group consisting of H, C1-4 alkyl, C2-4 alkenyl, C2-4 alkynyl, 5- or 6-membered cycloalkyl, 5- or 6-membered aryl, 5- or 6-membered heteroaryl, and 5- or 6-membered heterocyclyl, wherein each of the alkyl, alkenyl, alkynyl, cycloalkyl, aryl, heteroaryl, and heterocyclyl groups is optionally substituted with one, two or three substituents independently selected from the group consisting of C1-3 alkyl, halogen, —CF3, —CN, azido, —NO2, —OH, —O(C1-3 alkyl), —S(C1-3 alkyl), —NH2, —NH(C1-3 alkyl), —N(C1-3 alkyl)2, —NHS(O)2(C1-3 alkyl), —S(O)2NH2-z(C1-3 alkyl)z, —C(═O)OH, —C(═O)O(C1-3 alkyl), —C(═O)NH2-z(C1-3 alkyl)z, —NHC(═O)(C1-3 alkyl), —NHC(═NH)NHz-2(C1-3 alkyl)z, and —N(C1-3 alkyl)C(═NH)NH2-z(C1-3 alkyl)z, wherein each z is independently 0, 1, or 2 and each C1-3 alkyl is independently methyl, ethyl, A1, consisting of C1-3 alkyl, phenyl, halogen, —CF3, —OH, —OCH3, —SCH3, —NH2-z(CH3)z, —C(═O)OH, and —C(═O)OCH3, wherein z is 0, 1, or 2 and C1-3 alkyl is methyl, ethyl, propyl or isopropyl. More preferably, List A substituents are selected from List A2, consisting of methyl, ethyl, propyl, isopropyl, halogen (such as F, C1, or Br), and —CF3.

The term “amino acid” and “amino acid residue” may herein be used interchangeably, and are not to be understood limiting. Amino acids are organic compounds containing amine (—NH₂) and carboxyl (—COOH) functional groups, along with a side chain (R group) specific to each amino acid. In the context of the present disclosure, amino acids may be classified based on structure and chemical characteristics. Thus, classes of amino acids may be reflected in one or both of the following tables:

TABLE A
Main classification based on structure and general
chemical characterization of R group

| Class | Amino acid |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |

TABLE B
Alternative Physical and Functional Classifications
of Amino Acid Residues

| Class | Amino acid |
|---|---|
| Hydroxyl group containing residues | S and T |
| Aliphatic residues | I, L, V, and M |
| Cycloalkenyl-associated residues | F, H, W, and Y |
| Hydrophobic residues | A, C, F, G, H, I, L, M, |
| | R, T, V, W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved | A, C, D, E, G, H, K, |
| in turn formation | N, Q, R, S, P, and T |
| Flexible residues | Q, T, K, S, G, P, D, E, and R |

Unless indicated to the contrary, an “amino acid” or “amino acid residue” as used herein preferably refers to one of the 22 natural amino acids, 20 of which are listed in Table A (and are encoded by the universal genetic code) and the remaining 2 of which are selenocysteine and pyrrolysine (these 2 amino acids are incorporated into proteins by unique synthesis mechanisms). These 22 amino acids are also called “proteinogenic amino acids” or “natural amino acids”. The term “unnatural amino acid” as used herein refers to any amino acid which is not a natural amino acid (e.g., citrulline and homocysteine are examples of unnatural amino acids).

For the purposes of the present disclosure, “variants” of an amino acid sequence (peptide, protein or polypeptide) comprise amino acid insertion variants, amino acid addition variants, amino acid deletion variants and/or amino acid substitution variants. The term “variant” includes all mutants, splice variants, post-translationally modified variants, conformations, isoforms, allelic variants, species variants, and species homologs, in particular those which are naturally occurring. The term “variant” includes, in particular, fragments of an amino acid sequence.

Amino acid insertion variants comprise insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible.

Amino acid addition variants comprise amino- and/or carboxy-terminal fusions of one or more amino acids, such as 1, 2, 3, 5, 10, 20, 30, 50, or more amino acids.

Amino acid deletion variants are characterized by the removal of one or more amino acids from the sequence, such as by removal of 1, 2, 3, 5, 10, 20, 30, 50, or more amino acids. The deletions may be in any position of the protein. Amino acid deletion variants that comprise the deletion at the N-terminal and/or C-terminal end of the protein are also called N-terminal and/or C-terminal truncation variants.

Amino acid substitution variants are characterized by at least one residue in the sequence being removed and another residue being inserted in its place. Substitution of one amino acid for another may be classified as a conservative or non-conservative substitution. Preference is given to the modifications being in positions in the amino acid sequence which are not conserved between homologous proteins or peptides and/or to replacing amino acids with other ones having similar properties. Preferably, amino acid changes in peptide and protein variants are conservative amino acid changes, i.e., substitutions of similarly charged or uncharged amino acids. A conservative amino acid change involves substitution of one of a family of amino acids which are related in their side chains. In the context of the present disclosure, a “conservative substitution” is a substitution of one amino acid with another amino acid having similar structural and/or chemical characteristics, such substitution of one amino acid residue for another amino acid residue of the same class as defined in any of the two tables above: for example, leucine may be substituted with isoleucine as they are both aliphatic, branched hydrophobes. Similarly, aspartic acid may be substituted with glutamic acid since they are both small, negatively charged residues. Naturally occurring amino acids may also be generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In one embodiment, conservative amino acid substitutions include substitutions within the following groups: glycine, alanine; valine, isoleucine, leucine; aspartic acid, glutamic acid; asparagine, glutamine; serine, threonine; lysine, arginine; and phenylalanine, tyrosine.

Exemplary variants include those which differ from the VH and/or VL and/or CDRs of the parent sequences mainly by conservative substitutions; for example, 12, such as 11, 10, 9, 8, 7, 6, 5, 4, 3, 2 or 1 of the substitutions in the variant are conservative amino acid residue replacements. In the context of the present disclosure, conservative substitutions may be defined by substitutions within the classes of amino acids as defined in Tables A and B.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The percent identity between two nucleotide or amino acid sequences may e.g., be determined using the algorithm of E. Meyers and W. Miller, Comput. Appl. Biosci 4, 11-17 (1988) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch, J. Mol. Biol. 48, 444-453 (1970) algorithm.

Preferably the degree of similarity, preferably identity between a given amino acid sequence and an amino acid sequence which is a variant of said given amino acid sequence will be at least about 60%, 70%, 80%, 81%, 82%, 83%, 84%, 85%, 86%, 87%, 88%, 89%, 90%, 91%, 92%, 93%, 94%, 95%, 96%, 97%, 98%, or 99%. The degree of similarity or identity is given preferably for an amino acid region which is at least about 10%, at least about 20%, at least about 30%, at least about 40%, at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given preferably for at least about 20, at least about 40, at least about 60, at least about 80, at least about 100, at least about 120, at least about 140, at least about 160, at least about 180, or about 200 amino acids, in some embodiments continuous amino acids. In some embodiments, the degree of similarity or identity is given for the entire length of the reference amino acid sequence. The alignment for determining sequence similarity, preferably sequence identity can be done with art known tools, preferably using the best sequence alignment, for example, using Align, using standard settings, preferably EMBOSS:needle, Matrix: Blosum62, Gap Open 10.0, Gap Extend 0.5.

The disclosure describes nucleic acid sequences and amino acid sequences having a certain degree of similarity or identity to a given nucleic acid sequence or amino acid sequence, respectively (a reference sequence). “Sequence similarity” indicates the percentage of amino acids that either are identical or that represent conservative amino acid substitutions. “Sequence identity” between two amino acid sequences indicates the percentage of amino acids that are identical between the sequences. “Sequence identity” between two nucleic acid sequences indicates the percentage of nucleotides that are identical between the sequences.

The terms “% identical” and “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.). In some embodiments, percent identity of two sequences is determined using the BLASTN or BLASTP algorithm, as available on the United States National Center for Biotechnology Information (NCBI) website (e.g., at blast.ncbi.nlm.nih.gov/Blast.cgi). In some embodiments, the algorithm parameters used for BLASTN algorithm on the NCBI website include: (i) Expect Threshold set to 10; (ii) Word Size set to 28; (iii) Max matches in a query range set to 0; (iv) Match/Mismatch Scores set to 1, −2; (v) Gap Costs set to Linear; and (vi) the filter for low complexity regions being used. In some embodiments, the algorithm parameters used for BLASTP algorithm on the NCBI website include: (i) Expect Threshold set to 10; (ii) Word Size set to 3; (iii) Max matches in a query range set to 0; (iv) Matrix set to BLOSUM62; (v) Gap Costs set to Existence: 11 Extension: 1; and (vi) conditional compositional score matrix adjustment. Percentage identity may be obtained by determining the number of identical positions at which the sequences to be compared correspond, dividing this number by the number of positions compared (e.g., the number of positions in the reference sequence) and multiplying this result by 100.

In some embodiments, the degree of similarity or identity is given for a region which is at least about 50%, at least about 60%, at least about 70%, at least about 80%, at least about 90% or about 100% of the entire length of the reference sequence. For example, if the reference amino acid sequence consists of 200 amino acid residues, the degree of identity is given for at least about 100, at least about 120, at least about 140, at least about 160, at least about 180, or about 200 amino acid residues, in some embodiments continuous amino acid residues. In some embodiments, the degree of similarity or identity is given for the entire length of the reference sequence.

Nucleic acid sequences or amino acid sequences having a particular degree of similarity or identity to a given nucleic acid sequence or amino acid sequence, respectively, may have at least one functional property of said given sequence, e.g., and in some instances, are functionally equivalent to said given sequence. One important property includes an immunogenic property, in particular when administered to a subject. In some embodiments, a nucleic acid sequence or amino acid sequence having a particular degree of similarity or identity to a given nucleic acid sequence or amino acid sequence is functionally equivalent to the given sequence.

Homologous amino acid sequences exhibit according to the present disclosure at least 40%, in particular at least 50%, at least 60%, at least 70%, at least 80%, at least 90% and preferably at least 95%, at least 98 or at least 99% identity of the amino acid residues.

The resistance to, failure to respond to and/or relapse from treatment with a therapeutic agent (e.g., the anti-PD-1/PD-L1xVEGF/VEGFR binding agent or ADC) of the present disclosure may be determined according to the Response Evaluation Criteria in Solid Tumors; version 1.1 (RECIST Criteria v1.1). The RECIST Criteria are set forth in the Table C below (LD: longest dimension).

TABLE C
Definition of Response (RECIST Criteria v1.1)

| | Category | Criteria |
|---|---|---|
| Based on target | Complete | Disappearance of all target lesions. Any pathological |
| lesions | Response (CR) | lymph nodes must have reduction in short axis to <10 |
| | | mm. |
| | Partial Response | ≥30% decrease in the sum of the LD of target lesions, |
| | (PR) | taking as reference the baseline sum LD. |
| | Stable Disease | Neither sufficient shrinkage to qualify for PR nor |
| | (SD) | sufficient increase to qualify for PD, taking as reference |
| | | the smallest sum of LDs since the treatment started. |
| | Progressive | ≥20% increase in the sum of the LDs of target lesions, |
| | Disease (PD) | taking as reference the smallest sum of the LDs |
| | | recorded since the treatment started or the appearance |
| | | of one or more new lesions. |
| Based on non- | CR | Disappearance of all non-target lesions and |
| target lesions | | normalization of tumor marker level. All lymph nodes |
| | | must be non-pathological in size (<10 mm short axis). |
| | SD | Persistence of one or more non-target lesion(s) or/and |
| | | maintenance of tumor marker level above the normal |
| | | limits. |
| | PD | Appearance of one or more new lesions and/or |
| | | unequivocal progression of existing non-target lesions. |

The “best overall response” is the best response recorded from the start of the treatment until disease progression/recurrence (the smallest measurements recorded since the treatment started will be used as the reference for PD). Subjects with CR or PR are considered to be objective response. Subjects with CR, PR or SD are considered to be in disease control.

Subjects with NE are counted as non-responders. The best overall response is the best response recorded from the start of the treatment until disease progression/recurrence (the smallest measurements recorded since the treatment started will be used as the reference for PD). Subjects with CR, PR or SD are considered to be in disease control. Subjects with NE are counted as non-responders.

The objective response rate (ORR) is the percentage of all subjects in a study or treatment group who have either a partial or complete response to the treatment. The ORR can be calculated by adding the numbers of subjects having CR and the numbers of subjects having PR and dividing the resulting sum by the total number of subjects in the treatment group. The ORRₑᵥₐₗ, i.e., the ORR of all evaluable subjects in a study or treatment group, is the percentage of all evaluable subjects in a study or treatment group who have either a partial or complete response to the treatment.

The disease control rate (DCR) is the percentage of all subjects in a study or treatment group who have either a complete response, a partial response, or a stable disease to the treatment (CR, PR or SD). The DCR can be calculated by adding the numbers of subjects having CR, the numbers of subjects having PR, and the numbers of subjects having SD and dividing the resulting sum by the total number of subjects in the treatment group. The DCRₑᵥₐₗ, i.e., the DCR of all evaluable subjects in a study or treatment group, is the percentage of all evaluable subjects in a study or treatment group who have either a complete response, a partial response, or a stable disease to the treatment (CR, PR or SD).

“Duration of response (DOR)” only applies to subjects whose confirmed best overall response is CR or PR and is defined as the time from the first documentation of objective tumor response (CR or PR) to the date of first PD or death due to underlying cancer.

“Progression-free survival (PFS)” is defined as the number of days from Day 1 in Cycle 1 to the first documented progression or death due to any cause.

“Overall survival (OS)” is defined as the number of days from Day 1 in Cycle 1 to death due to any cause. If a subject is not known to have died, then OS will be censored at the latest date the subject was known to be alive (on or before the cut-off date).

Recitation of ranges of values herein is merely intended to serve as a shorthand method of referring individually to each separate value falling within the range. Unless otherwise indicated herein, each individual value is incorporated into the specification as if it was individually recited herein. All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

All patents, patent applications, and other publications cited in this application are incorporated by reference in the entirety for all purposes. Nothing herein is to be construed as an admission that the invention is not entitled to antedate such disclosure by virtue of prior invention.

### The Combination Therapy of the Invention

In some aspects of the invention, there is provided a combination of
a. a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction; and
b. an antibody-drug conjugate comprising an antibody or fragment thereof that specifically binds to a target expressed on the surface of a tumour cell.

In some aspects of the invention, there is provided a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction;
for use in treating cancer in a subject, the use comprising administering to the subject:
a. the binding agent; and
b. an antibody-drug conjugate comprising an antibody or fragment thereof that specifically binds to a target expressed on the surface of a tumour cell.

In some aspects of the invention, there is provided an antibody-drug conjugate comprising an antibody or fragment thereof that specifically binds to a target expressed on the surface of a tumour cell for use in treating cancer in a subject, the use comprising administering to the subject:
a. a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction; and
b. the antibody-drug conjugate.

In some aspects of the invention, there is provided an antibody-drug conjugate comprising an antibody or fragment thereof that specifically binds to a target expressed on the surface of a tumour cell and a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction,
for use in therapy, preferably for treating cancer in a subject, preferably the use comprising administering to the subject:
a. the binding agent; and
b. the antibody-drug conjugate.

In some aspects of the invention, there is provided a combination of
a. a bispecific antibody that comprises
(a) an antibody or fragment thereof that specifically binds to PD-L1 and
(b) an antibody or fragment thereof that specifically binds to VEGF; and
b. an antibody-drug conjugate comprising an antibody or fragment thereof that specifically binds to a target expressed on the surface of a tumour cell.

In some aspects of the invention, there is provided a bispecific antibody that comprises
(a) an antibody or fragment thereof that specifically binds to PD-L1, and
(b) an antibody or fragment thereof that specifically binds to VEGF;
for use in treating cancer in a subject, the use comprising administering to the subject:
a. the bispecific antibody; and
b. an antibody-drug conjugate comprising an antibody or fragment thereof that specifically binds to a target expressed on the surface of a tumour cell.

In some aspects of the invention, there is provided an antibody-drug conjugate comprising an antibody or fragment thereof that specifically binds to a target expressed on the surface of a tumour cell for use in treating cancer in a subject, the use comprising administering to the subject:
a. a bispecific antibody that comprises
(a) an antibody or fragment thereof that specifically binds to PD-L1, and
(b) an antibody or fragment thereof that specifically binds to VEGF; and
b. the antibody-drug conjugate.

In some aspects of the invention, there is provided, an antibody-drug conjugate comprising an antibody or fragment thereof that specifically binds to a target expressed on the surface of a tumour cell and a bispecific antibody that comprises
(a) an antibody or fragment thereof that specifically binds to PD-L1, and
(b) an antibody or fragment thereof that specifically binds to VEGF, for use in therapy, preferably for treating cancer in a subject, preferably the use comprising administering to the subject:
a. the bispecific antibody; and
b. the antibody-drug conjugate.

### Antibody-Drug Conjugate Components

The combination therapy of the present invention comprises an antibody-drug conjugate comprising an antibody or fragment thereof that specifically binds to a target expressed on the surface of a tumour cell. It will be understood by the skilled person that the term “antibody-drug conjugate” encompasses both specific antibody-drug conjugates having a DAR (as defined below) which is integral, and compositions (as defined below) containing a mixture of antibody-drug conjugates having different DARs, resulting in an average DAR (as defined below) which can be non-integral.

Targets which are expressed on the surface of a tumour cell may be identified by means of various molecular biological and immunological methods known in the art (see e.g. Carter, P, et al., 2004. Endocrine-related cancer, 11(4), pp. 659-687). An ideal expression profile is abundant and homogeneous antigen expression on the external surface of all tumour cells for multiple tumour types with the majority of patients for each tumour type, and absent from normal tissue. In some embodiments, the target is expressed on the surface of solid tumour cells.

In some embodiments, the target is expressed with higher expression on the surface of tumour cells as compared to healthy cells. In some embodiments, the target is expressed at a level sufficient to distinguish a tumour cell from healthy cells. Evaluation of antigen expression on the surface may be determined by any suitable method, for example by immunohistochemistry e.g. tissue microarray immunohistochemistry (see e.g. Hoos, A. and Cordon-Cardo, C, 2001. Laboratory investigation, 81(10), pp. 1331-1338).

Example targets may include B7H3, TROP2, HER2, HER3, ALK receptor (pleiotrophin receptor); ovarian carcinoma antigen (CA125); prostate-specific antigen (PSA); melanoma-associated antigen p97; melanoma antigen gp75; high molecular weight melanoma antigen (HMW-MAA); prostate-specific membrane antigen (PSMA); carcinoembryonic antigen (CEA); polymorphic epithelial mucin antigen; CD19; human B-lymphoma antigen-CD20; CD33; melanoma-specific antigens such as ganglioside GD2, ganglioside GD3, ganglioside GM2, and ganglioside GM3; epidermal growth factor receptor (EGFR); NY—BR-16; NY—BR-16; polymorphic epithelial mucin (PEM); malignant human lymphocyte antigen-APO-1; SSEA-1; SCP-1; SSEA-3; SSEA-4; sialyl Tn (STn); colon cancer antigen NY—CO-45; lung cancer antigen NY-LU-12 variant A; adenocarcinoma antigen ART1; Neuro-oncological ventral antigen 2 (NOVA2); tumor-associated antigens MAGE-C1 (cancer/testis antigen CT7), MAGE-B1 (MAGE-XP antigen), MAGE-B2 (DAM6), MAGE-2, MAGE-4a, MAGE-4b and MAGE-X2; Cancer-Testis Antigen (NY-EOS-1); YKL-40; EpCAM; EREG; CA15-3; sialyl SSEA-1(SLX); CLDN6; CLDN18.2 and Mesothelin. Other example targets may include CD30, Nectin-4, TF, B7-H4 and ITGB6. In some embodiments, the target is selected from B7H3, TROP2, HER2, and HER3.

In some embodiments, the target expressed on the surface of a tumour cell is HER2, B7H3, TROP2, HER3, CD30, Nectin-4, TF, B7-H4, and/or ITGB6.

In some preferred embodiments, the target expressed on the surface of a tumour cell is HER2, B7H3, TROP2 and/or HER3. In some preferred embodiments, the target expressed on the surface of a tumour cell is HER2, B7H3 or TROP2. In some preferred embodiments, the target expressed on the surface of a tumour cell is HER3.

In other embodiments, the target expressed on the surface of a tumour cell is CD30, Nectin-4, TF, B7-H4, ITGB6, and/or HER2. In some embodiments, the target expressed on the surface of a tumour cell is CD30, Nectin-4, TF, B7-H4, ITGB6, or HER2.

The antibody or fragment thereof that specifically binds to a target expressed on the surface of a tumour cell may be selected from the group consisting of a human antibody, a humanized antibody, a chimeric antibody, a multispecific antibody, a monoclonal antibody, a polyclonal antibody, and fragments thereof. It will be understood by the skilled person that the term “antibody-drug conjugate” applies irrespective of whether the binding moiety is an antibody or a fragment thereof.

In some embodiments, the antibody or fragment thereof that specifically binds to a target expressed on the surface of a tumour cell is a full-length antibody, such as a human antibody, a humanized antibody, a chimeric antibody, a multispecific antibody, a monoclonal antibody, or a polyclonal antibody. In some embodiments, the antibody or fragment thereof that specifically binds to a target expressed on the surface of a tumour cell is a human antibody, a humanized antibody, or a chimeric antibody. In some embodiments, the antibody or fragment thereof that specifically binds to a target expressed on the surface of a tumour cell is a monoclonal antibody, for example a monoclonal human antibody, a monoclonal humanized antibody, or a monoclonal chimeric antibody.

In other embodiments, the antibody or fragment thereof that specifically binds to a target expressed on the surface of a tumour cell is an antibody fragment, such as a Fab, a Fab′, a F(ab′)2, an Fv, an scFv, a Fab′-SH, an sdAb, or a VHH.

In some embodiments, the antibody or fragment thereof comprises an immunoglobulin constant region. In some embodiments, the immunoglobulin constant region is a human IgG constant region, for example a human IgG1, IgG2, IgG3 or IgG4 constant region. In some embodiments, the immunoglobulin constant region is a human IgG1 constant region.

In some embodiments, the antibody or fragment thereof comprises an Fc region. In some embodiments, the Fc region is a human Fc region, for example a human IgG1, IgG2, IgG3 or IgG4 Fc region. In some embodiments, the antibody or fragment thereof comprises a human IgG1 Fc region.

In some embodiments, the antibody or fragment thereof comprises an Fc region comprising one or more mutations which reduce or abolish immune effect functions. Suitable mutations are known in the art and may include one or more amino acid substitutions (see e.g. Wilkinson, I., et al., 2021. FcPLoS One, 16(12), p.e0260954; and Liu, R., et al., 2020. Antibodies, 9(4), p.64). In some embodiments, the antibody or fragment thereof comprises an IgG1 Fc region comprising a L234A and/or a L235A mutation. In some embodiments, the antibody or fragment thereof comprises an IgG1 Fc region comprising L234A and L235A mutations. Amino acids are numbered according to the Eu numbering (Eu-index) as set forth in Kabat (Kabat, E. A.; National Institutes of Health (U.S.) Office of the Director. Sequences of Proteins of Immunological Interest, 5th ed.; DIANE Publishing: Collingdale, PA, USA, 1991).

The antibody-drug conjugate may be formulated for administration to a subject. The antibody-drug conjugate may be formulated as a liquid, a solid, or a combination thereof. In some embodiments, the antibody-drug conjugate is formulated as a liquid. The antibody-drug conjugate may be formulated for injection or infusion. In some embodiments, antibody-drug conjugate is formulated for intravenous administration.

### Anti-B7H3 Antibodies and Fragments Thereof.

In some embodiments, the target expressed on the surface of a tumour cell is B7H3. B7H3 is a protein that in humans is encoded by the CD276 gene and which may also be known as Cluster of Differentiation 276 (CD276) and B7 Homolog 3 (B7-H3).

In some embodiments, the antibody-drug conjugate comprises an anti-B7H3 antibody or fragment thereof.

The anti-B7H3 antibody or fragment thereof may specifically bind B7H3. The anti-B7H3 antibody may be a monoclonal antibody. In some embodiments, the anti-B7H3 antibody or fragment thereof is a humanized antibody. In some embodiments, the anti-B7H3 antibody or fragment thereof is a monoclonal humanized antibody. The fragment thereof may be any antigen-binding fragment thereof, for example a Fab, a Fab′, a F(ab′)2, a Fv, a scFv, a Fab′-SH, an sdAb, or a VHH. In some embodiments, the anti-B7H3 antibody or fragment thereof is a full-length anti-B7H3 antibody.

The anti-B7H3 antibody or fragment thereof may comprise a variable region that specifically binds B7H3. In some embodiments, the anti-B7H3 antibody or fragment thereof comprises a heavy chain variable region and/or a light chain variable domain. In some embodiments, the anti-B7H3 antibody or fragment thereof comprises a heavy chain variable region and a light chain variable domain. In some embodiments, the anti-B7H3 antibody or fragment thereof comprises a constant region, preferably derived from a human antibody, preferably the constant region is selected from the constant region of human IgG1, IgG2, IgG3 or IgG4. In some embodiments, the anti-B7H3 antibody or fragment thereof comprises a heavy chain and/or a light chain. In some embodiments, the anti-B7H3 antibody or fragment thereof comprises a heavy chain and a light chain. In some embodiments, the anti-B7H3 antibody or fragment thereof comprises or consists of two heavy chains and two light chains.

Example anti-B7H3 antibody CDRs, variable region sequences, and heavy and light chain sequences are provided below.

Example anti-B7H3 Antibody Sequences 1

| | |
|---|---|
| Kabat HCDR1 | GDYAWN (SEQ ID NO: 1) |
| Kabat HCDR2 | YISYSGSTSYNPSLQS (SEQ ID NO: 2) |
| Kabat HCDR3 | SLGRRWYFVV (SEQ ID NO: 3) |
| Kabat LCDR1 | KSSQSLLNSSNQKNYLA (SEQ ID NO: 4) |
| Kabat LCDR2 | FASTRES (SEQ ID NO: 5) |
| Kabat LCDR3 | QQHYSAPWT (SEQ ID NO: 6) |
| IMGT HCDR1 | DYSITGDYA (SEQ ID NO: 7) |
| IMGT HCDR2 | ISYSGST (SEQ ID NO: 8) |
| IMGT HCDR3 | ARSLGRRWYFVV (SEQ ID NO: 9) |
| IMGT LCDR1 | QSLLNSSNQKNY (SEQ ID NO: 10) |
| IMGT LCDR2 | FAS (SEQ ID NO: 11) |
| IMGT LCDR3 | QQHYSAPWT (SEQ ID NO: 12) |
| VH | DVQLQESGPGLVKPSQSLSLTCTVTDYSITGDYAWNWIRQFPGNKLE |
| | WMGYISYSGSTSYNPSLQSRISITRDTSKNQFFLQLNSVTSEDTATYF |
| | CARSLGRRWYFVVWGAGTTVTVSA (SEQ ID NO: 13) |
| VL | DIVMTQSPSSLAMSVGQKVTMSCKSSQSLLNSSNQKNYLAWYQQKP |
| | GQSPKLLIYFASTRESGVPDRFIGSGSGTDFTLTISSVQAEDLTDYFCQ |
| | QHYSAPWTFGGGTKLEIK (SEQ ID NO: 14) |
| Heavy chain | DVQLQESGPGLVKPSQSLSLTCTVTDYSITGDYAWNWIRQFPGNKLE |
| | WMGYISYSGSTSYNPSLQSRISITRDTSKNQFFLQLNSVTSEDTATYF |
| | CARSLGRRWYFVVWGAGTTVTVSAASTKGPSVFPLAPSSKSTSGGT |
| | AALGCLVKDYFPEPVTVSWNSGALTSGVHTFPAVLQSSGLYSLSSVV |
| | TVPSSSLGTQTYICNVNHKPSNTKVDKRVEPKSCDKTHTCPPCPAPE |
| | AAGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSN |
| | KALPAPIEKTISKAKGQPREPQVYTLPPSREEMTKNQVSLTCLVKGFY |
| | PSDIAVEWESNGQPENNYKTTPPVLDSDGSFFLYSKLTVDKSRWQQG |
| | NVFSCSVMHEALHNHYTQKSLSLSPG (SEQ ID NO: 15) |
| Light chain | DIVMTQSPSSLAMSVGQKVTMSCKSSQSLLNSSNQKNYLAWYQQKP |
| | GQSPKLLIYFASTRESGVPDRFIGSGSGTDFTLTISSVQAEDLTDYFCQ |
| | QHYSAPWTFGGGTKLEIKRTVAAPSVFIFPPSDEQLKSGTASVVCLLN |
| | NFYPREAKVQWKVDNALQSGNSQESVTEQDSKDSTYSLSSTLTLSK |
| | ADYEKHKVYACEVTHQGLSSPVTKSFNRGEC (SEQ ID NO: 16) |

In some embodiments, a heavy chain variable region of the anti-B7H3 antibody or fragment thereof comprises: (i) a HCDR1 comprising or consisting of the amino acid sequence of SEQ ID NO: 1, or a variant thereof having up to three amino acid substitutions, additions or deletions; (ii) a HCDR2 comprising or consisting of the amino acid sequence of SEQ ID NO: 2, or a variant thereof having up to three amino acid substitutions, additions or deletions; and (iii) a HCDR3 comprising or consisting of the amino acid sequence of SEQ ID NO: 3, or a variant thereof having up to three amino acid substitutions, additions or deletions, wherein the CDRs are defined according to the Kabat numbering system.

In some embodiments, a light chain variable region of the anti-B7H3 antibody or fragment thereof comprises: (i) a LCDR1 comprising or consisting of the amino acid sequence of SEQ ID NO: 4, or a variant thereof having up to three amino acid substitutions, additions or deletions; (ii) a LCDR2 comprising or consisting of the amino acid sequence of SEQ ID NO: 5, or a variant thereof having up to three amino acid substitutions, additions or deletions; and (iii) a LCDR3 comprising or consisting of the amino acid sequence of SEQ ID NO: 6, or a variant thereof having up to three amino acid substitutions, additions or deletions, wherein the CDRs are defined according to the Kabat numbering system.

In some embodiments, a heavy chain variable region of the anti-B7H3 antibody or fragment thereof comprises: (i) a HCDR1 comprising or consisting of the amino acid sequence of SEQ ID NO: 1, or a variant thereof having up to three amino acid substitutions, additions or deletions; (ii) a HCDR2 comprising or consisting of the amino acid sequence of SEQ ID NO: 2, or a variant thereof having up to three amino acid substitutions, additions or deletions; and (iii) a HCDR3 comprising or consisting of the amino acid sequence of SEQ ID NO: 3, or a variant thereof having up to three amino acid substitutions, additions or deletions; and a light chain variable region of the anti-B7H3 antibody or fragment thereof comprises: (i) a LCDR1 comprising or consisting of the amino acid sequence of SEQ ID NO: 4, or a variant thereof having up to three amino acid substitutions, additions or deletions; (ii) a LCDR2 comprising or consisting of the amino acid sequence of SEQ ID NO: 5, or a variant thereof having up to three amino acid substitutions, additions or deletions; and (iii) a LCDR3 comprising or consisting of the amino acid sequence of SEQ ID NO: 6, or a variant thereof having up to three amino acid substitutions, additions or deletions, wherein the CDRs are defined according to the Kabat numbering system.

In some embodiments, a heavy chain variable region of the anti-B7H3 antibody or fragment thereof comprises: (i) a HCDR1 comprising or consisting of the amino acid sequence of SEQ ID NO: 7, or a variant thereof having up to three amino acid substitutions, additions or deletions; (ii) a HCDR2 comprising or consisting of the amino acid sequence of SEQ ID NO: 8, or a variant thereof having up to three amino acid substitutions, additions or deletions; and (iii) a HCDR3 comprising or consisting of the amino acid sequence of SEQ ID NO: 9, or a variant thereof having up to three amino acid substitutions, additions or deletions, wherein the CDRs are defined according to the IMGT numbering system.

In some embodiments, a light chain variable region of the anti-B7H3 antibody or fragment thereof comprises: (i) a LCDR1 comprising or consisting of the amino acid sequence of SEQ ID NO: 10, or a variant thereof having up to three amino acid substitutions, additions or deletions; (ii) a LCDR2 comprising or consisting of the amino acid sequence of SEQ ID NO: 11; and (iii) a LCDR3 comprising or consisting of the amino acid sequence of SEQ ID NO: 12, or a variant thereof having up to three amino acid substitutions, additions or deletions, wherein the CDRs are defined according to the IMGT numbering system.

In some embodiments, a heavy chain variable region of the anti-B7H3 antibody or fragment thereof comprises: (i) a HCDR1 comprising or consisting of the amino acid sequence of SEQ ID NO: 7, or a variant thereof having up to three amino acid substitutions, additions or deletions; (ii) a HCDR2 comprising or consisting of the amino acid sequence of SEQ ID NO: 8, or a variant thereof having up to three amino acid substitutions, additions or deletions; and (iii) a HCDR3 comprising or consisting of the amino acid sequence of SEQ ID NO: 9, or a variant thereof having up to three amino acid substitutions, additions or deletions; and a light chain variable region of the anti-B7H3 antibody or fragment thereof comprises: (i) a LCDR1 comprising or consisting of the amino acid sequence of SEQ ID NO: 10, or a variant thereof having up to three amino acid substitutions, additions or deletions; (ii) a LCDR2 comprising or consisting of the amino acid sequence of SEQ ID NO: 11, or a variant thereof having up to three amino acid substitutions, additions or deletions; and (iii) a LCDR3 comprising or consisting of the amino acid sequence of SEQ ID NO: 12, or a variant thereof having up to three amino acid substitutions, additions or deletions, wherein the CDRs are defined according to the IMGT numbering system.

In some embodiments, the CDR variants have up to two amino acid substitutions, additions or deletions. In some embodiments, the CDR variants have up to one amino acid substitution, addition or deletion. In some embodiments, the CDR variants have up to three amino acid substitutions. In some embodiments, the CDR variants have up to two amino acid substitutions. In some embodiments, the CDR variants have up to one amino acid substitution.

In some embodiments, a heavy chain variable region of the anti-B7H3 antibody or fragment thereof comprises or consists of the amino acid sequence of SEQ ID NO: 13, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof. In some embodiments, a heavy chain variable region of the anti-B7H3 antibody or fragment thereof comprises or consists of the amino acid sequence of SEQ ID NO: 13.

In some embodiments, a light chain variable region of the anti-B7H3 antibody or fragment thereof comprises or consists of the amino acid sequence of SEQ ID NO: 14 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof. In some embodiments, a light chain variable region of the anti-B7H3 antibody or fragment thereof comprises or consists of the amino acid sequence of SEQ ID NO: 14.

In some embodiments, a heavy chain variable region of the anti-B7H3 antibody or fragment thereof comprises or consists of the amino acid sequence of SEQ ID NO: 13, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, and a light chain variable region of the anti-B7H3 antibody or fragment thereof comprises or consists of the amino acid sequence of SEQ ID NO: 14 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof.

In some embodiments, a heavy chain variable region of the anti-B7H3 antibody or fragment thereof comprises or consists of the amino acid sequence of SEQ ID NO: 13, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the heavy chain variable region comprises: a HCDR1 comprising or consisting of the amino acid sequence of SEQ ID NO: 1; a HCDR2 comprising or consisting of the amino acid sequence of SEQ ID NO: 2; and a HCDR3 comprising or consisting of the amino acid sequence of SEQ ID NO: 3; and a light chain variable region of the anti-B7H3 antibody or fragment thereof comprises or consists of the amino acid sequence of SEQ ID NO: 14 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the light chain variable region comprises: a LCDR1 comprising or consisting of the amino acid sequence of SEQ ID NO: 4; a LCDR2 comprising or consisting of the amino acid sequence of SEQ ID NO: 5; and a LCDR3 comprising or consisting of the amino acid sequence of SEQ ID NO: 6.

In some embodiments, a heavy chain variable region of the anti-B7H3 antibody or fragment thereof comprises or consists of the amino acid sequence of SEQ ID NO: 13, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the heavy chain variable region comprises: a HCDR1 comprising or consisting of the amino acid sequence of SEQ ID NO: 7; a HCDR2 comprising or consisting of the amino acid sequence of SEQ ID NO: 8; and a HCDR3 comprising or consisting of the amino acid sequence of SEQ ID NO: 9; and a light chain variable region of the anti-B7H3 antibody or fragment thereof comprises or consists of the amino acid sequence of SEQ ID NO: 14 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the light chain variable region comprises: a LCDR1 comprising or consisting of the amino acid sequence of SEQ ID NO: 10; a LCDR2 comprising or consisting of the amino acid sequence of SEQ ID NO: 11; and a LCDR3 comprising or consisting of the amino acid sequence of SEQ ID NO: 12.

In some embodiments, a heavy chain variable region of the anti-B7H3 antibody or fragment thereof comprises or consists of the amino acid sequence of SEQ ID NO: 13; and a light chain variable region of the anti-B7H3 antibody or fragment thereof comprises or consists of the amino acid sequence of SEQ ID NO: 14. In some embodiments, a heavy chain variable region of the anti-B7H3 antibody or fragment thereof consists of the amino acid sequence of SEQ ID NO: 13; and a light chain variable region of the anti-B7H3 antibody or fragment thereof consists of the amino acid sequence of SEQ ID NO: 14.

In some embodiments, a heavy chain of the anti-B7H3 antibody or fragment thereof comprises or consists of the amino acid sequence of SEQ ID NO: 15, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof. In some embodiments, a heavy chain of the anti-B7H3 antibody or fragment thereof comprises or consists of the amino acid sequence of SEQ ID NO: 15.

In some embodiments, a light chain of the anti-B7H3 antibody or fragment thereof comprises or consists of the amino acid sequence of SEQ ID NO: 16 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof. In some embodiments, a light chain of the anti-B7H3 antibody or fragment thereof comprises or consists of the amino acid sequence of the amino acid sequence of SEQ ID NO: 16.

In some embodiments, a heavy chain of the anti-B7H3 antibody or fragment thereof comprises or consists of the amino acid sequence of the amino acid sequence of SEQ ID NO: 15, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof; and a light chain of the anti-B7H3 antibody or fragment thereof comprises or consists of the amino acid sequence of SEQ ID NO: 16 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof.

In some embodiments, a heavy chain of the anti-B7H3 antibody or fragment thereof comprises or consists of the amino acid sequence of SEQ ID NO: 15, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the heavy chain variable region comprises: a HCDR1 comprising or consisting of the amino acid sequence of SEQ ID NO: 1; a HCDR2 comprising or consisting of the amino acid sequence of SEQ ID NO: 2; and a HCDR3 comprising or consisting of the amino acid sequence of SEQ ID NO: 3; and a light chain of the anti-B7H3 antibody or fragment thereof comprises or consists of the amino acid sequence of SEQ ID NO: 16 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the light chain variable region comprises: a LCDR1 comprising or consisting of the amino acid sequence of SEQ ID NO: 4; a LCDR2 comprising or consisting of the amino acid sequence of SEQ ID NO: 5; and a LCDR3 comprising or consisting of the amino acid sequence of SEQ ID NO: 6.

In some embodiments, a heavy chain of the anti-B7H3 antibody or fragment thereof comprises or consists of the amino acid sequence of SEQ ID NO: 15, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the heavy chain variable region comprises: a HCDR1 comprising or consisting of the amino acid sequence of SEQ ID NO: 7; a HCDR2 comprising or consisting of the amino acid sequence of SEQ ID NO: 8; and a HCDR3 comprising or consisting of the amino acid sequence of SEQ ID NO: 9; and a light chain of the anti-B7H3 antibody or fragment thereof comprises or consists of the amino acid sequence of SEQ ID NO: 16 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the light chain variable region comprises: a LCDR1 comprising or consisting of the amino acid sequence of SEQ ID NO: 10; a LCDR2 comprising or consisting of the amino acid sequence of SEQ ID NO: 11; and a LCDR3 comprising or consisting of the amino acid sequence of SEQ ID NO: 12.

In some embodiments, a heavy chain of the anti-B7H3 antibody or fragment thereof comprises or consists of the amino acid sequence of SEQ ID NO: 15; and a light chain of the anti-B7H3 antibody or fragment thereof comprises or consists of the amino acid sequence of SEQ ID NO: 16. In some embodiments, a heavy chain of the anti-B7H3 antibody or fragment thereof consists of the amino acid sequence of SEQ ID NO: 15; and a light chain of the anti-B7H3 antibody or fragment thereof consists of the amino acid sequence of SEQ ID NO: 16.

In some embodiments, the anti-B7H3 antibody or fragment thereof comprises or consists of two heavy chains comprising or consisting of the amino acid sequence of SEQ ID NO: 15; and two light chain comprising or consisting of the amino acid sequence of SEQ ID NO: 16. In some embodiments, the anti-B7H3 antibody or fragment thereof comprises or consists of two heavy chains consisting of the amino acid sequence of SEQ ID NO: 15; and two light chains consisting of the amino acid sequence of SEQ ID NO: 16.

### Example Anti-B7H3 Antibody Sequences 2

| | |
|---|---|
| Kabat HCDR1 | GDYAWN (SEQ ID NO: 17) |
| Kabat HCDR2 | YISYSGSTSYNPSLQS (SEQ ID NO: 18) |
| Kabat HCDR3 | SLGRRWYFVV (SEQ ID NO: 19) |
| Kabat LCDR1 | KSSQSLLQSSNQKNYLA (SEQ ID NO: 20) |
| Kabat LCDR2 | FASTRES (SEQ ID NO: 21) |
| Kabat LCDR3 | QQHYSAPWT (SEQ ID NO: 22) |
| IMGT HCDR1 | DYSITGDYA (SEQ ID NO: 23) |
| IMGT HCDR2 | ISYSGST (SEQ ID NO: 24) |
| IMGT HCDR3 | ARSLGRRWYFVV (SEQ ID NO: 25) |
| IMGT LCDR1 | QSLLQSSNQKNY (SEQ ID NO: 26) |
| IMGT LCDR2 | FAS (SEQ ID NO: 27) |
| IMGT LCDR3 | QQHYSAPWT (SEQ ID NO: 28) |
| VH | QVQLQESGPGLVKPSQTLSLTCTVTDYSITGDYAWNWIRQHPGKGL |
| | EWIGYISYSGSTSYNPSLQSRVTISRDTSKNQFSLKLSSVTAADTAVY |
| | FCARSLGRRWYFVVWGQGTTVTVSS (SEQ ID NO: 29) |
| VL | DIVMTQSPDSLAVSLGERATINCKSSQSLLQSSNQKNYLAWYQQKPG |
| | QPPKLLIYFASTRESGVPDRFSGSGSGTDFTLTISSLQAEDVAVYYCQ |
| | QHYSAPWTFGGGTKVEIK (SEQ ID NO: 30) |
| Heavy chain | QVQLQESGPGLVKPSQTLSLTCTVTDYSITGDYAWNWIRQHPGKGL |
| | EWIGYISYSGSTSYNPSLQSRVTISRDTSKNQFSLKLSSVTAADTAVY |
| | FCARSLGRRWYFVVWGQGTTVTVSSASTKGPSVFPLAPSSKSTSGGT |
| | AALGCLVKDYFPEPVTVSWNSGALTSGVHTFPAVLQSSGLYSLSSVV |
| | TVPSSSLGTQTYICNVNHKPSNTKVDKRVEPKSCDKTHTCPPCPAPE |
| | AAGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSN |
| | KALPAPIEKTISKAKGQPREPQVYTLPPSREEMTKNQVSLTCLVKGFY |
| | PSDIAVEWESNGQPENNYKTTPPVLDSDGSFFLYSKLTVDKSRWQQG |
| | NVFSCSVMHEALHNHYTQKSLSLSPGK (SEQ ID NO: 31) |
| Light chain | DIVMTQSPDSLAVSLGERATINCKSSQSLLQSSNQKNYLAWYQQKPG |
| | QPPKLLIYFASTRESGVPDRFSGSGSGTDFTLTISSLQAEDVAVYYCQ |
| | QHYSAPWTFGGGTKVEIKRTVAAPSVFIFPPSDEQLKSGTASVVCLL |
| | NNFYPREAKVQWKVDNALQSGNSQESVTEQDSKDSTYSLSSTLTLS |
| | KADYEKHKVYACEVTHQGLSSPVTKSFNRGEC (SEQ ID NO: 32) |

In some embodiments, a heavy chain variable region of the anti-B7H3 antibody or fragment thereof comprises: (i) a HCDR1 comprising or consisting of the amino acid sequence of SEQ ID NO: 17, or a variant thereof having up to three amino acid substitutions, additions or deletions; (ii) a HCDR2 comprising or consisting of the amino acid sequence of SEQ ID NO: 18, or a variant thereof having up to three amino acid substitutions, additions or deletions; and (iii) a HCDR3 comprising or consisting of the amino acid sequence of SEQ ID NO: 19, or a variant thereof having up to three amino acid substitutions, additions or deletions, wherein the CDRs are defined according to the Kabat numbering system.

In some embodiments, a light chain variable region of the anti-B7H3 antibody or fragment thereof comprises: (i) a LCDR1 comprising or consisting of the amino acid sequence of SEQ ID NO: 20, or a variant thereof having up to three amino acid substitutions, additions or deletions; (ii) a LCDR2 comprising or consisting of the amino acid sequence of SEQ ID NO: 21, or a variant thereof having up to three amino acid substitutions, additions or deletions; and (iii) a LCDR3 comprising or consisting of the amino acid sequence of SEQ ID NO: 22, or a variant thereof having up to three amino acid substitutions, additions or deletions, wherein the CDRs are defined according to the Kabat numbering system.

In some embodiments, a heavy chain variable region of the anti-B7H3 antibody or fragment thereof comprises: (i) a HCDR1 comprising or consisting of the amino acid sequence of SEQ ID NO: 17, or a variant thereof having up to three amino acid substitutions, additions or deletions; (ii) a HCDR2 comprising or consisting of the amino acid sequence of SEQ ID NO: 18, or a variant thereof having up to three amino acid substitutions, additions or deletions; and (iii) a HCDR3 comprising or consisting of the amino acid sequence of SEQ ID NO: 19, or a variant thereof having up to three amino acid substitutions, additions or deletions; and a light chain variable region of the anti-B7H3 antibody or fragment thereof comprises: (i) a LCDR1 comprising or consisting of the amino acid sequence of SEQ ID NO: 20, or a variant thereof having up to three amino acid substitutions, additions or deletions; (ii) a LCDR2 comprising or consisting of the amino acid sequence of SEQ ID NO: 21, or a variant thereof having up to three amino acid substitutions, additions or deletions; and (iii) a LCDR3 comprising or consisting of the amino acid sequence of SEQ ID NO: 22, or a variant thereof having up to three amino acid substitutions, additions or deletions, wherein the CDRs are defined according to the Kabat numbering system.

In some embodiments, a heavy chain variable region of the anti-B7H3 antibody or fragment thereof comprises: (i) a HCDR1 comprising or consisting of the amino acid sequence of SEQ ID NO: 23, or a variant thereof having up to three amino acid substitutions, additions or deletions; (ii) a HCDR2 comprising or consisting of the amino acid sequence of SEQ ID NO: 24, or a variant thereof having up to three amino acid substitutions, additions or deletions; and (iii) a HCDR3 comprising or consisting of the amino acid sequence of SEQ ID NO: 25, or a variant thereof having up to three amino acid substitutions, additions or deletions, wherein the CDRs are defined according to the IMGT numbering system.

In some embodiments, a light chain variable region of the anti-B7H3 antibody or fragment thereof comprises: (i) a LCDR1 comprising or consisting of the amino acid sequence of SEQ ID NO: 26, or a variant thereof having up to three amino acid substitutions, additions or deletions; (ii) a LCDR2 comprising or consisting of the amino acid sequence of SEQ ID NO: 27; and (iii) a LCDR3 comprising or consisting of the amino acid sequence of SEQ ID NO: 28, or a variant thereof having up to three amino acid substitutions, additions or deletions, wherein the CDRs are defined according to the IMGT numbering system.

In some embodiments, a heavy chain variable region of the anti-B7H3 antibody or fragment thereof comprises: (i) a HCDR1 comprising or consisting of the amino acid sequence of SEQ ID NO: 23, or a variant thereof having up to three amino acid substitutions, additions or deletions; (ii) a HCDR2 comprising or consisting of the amino acid sequence of SEQ ID NO: 24, or a variant thereof having up to three amino acid substitutions, additions or deletions; and (iii) a HCDR3 comprising or consisting of the amino acid sequence of SEQ ID NO: 25, or a variant thereof having up to three amino acid substitutions, additions or deletions; and a light chain variable region of the anti-B7H3 antibody or fragment thereof comprises: (i) a LCDR1 comprising or consisting of the amino acid sequence of SEQ ID NO: 26, or a variant thereof having up to three amino acid substitutions, additions or deletions; (ii) a LCDR2 comprising or consisting of the amino acid sequence of SEQ ID NO: 27, or a variant thereof having up to three amino acid substitutions, additions or deletions; and (iii) a LCDR3 comprising or consisting of the amino acid sequence of SEQ ID NO: 28, or a variant thereof having up to three amino acid substitutions, additions or deletions, wherein the CDRs are defined according to the IMGT numbering system.

In some embodiments, the CDR variants have up to two amino acid substitutions, additions or deletions. In some embodiments, the CDR variants have up to one amino acid substitution, addition or deletion. In some embodiments, the CDR variants have up to three amino acid substitutions. In some embodiments, the CDR variants have up to two amino acid substitutions. In some embodiments, the CDR variants have up to one amino acid substitution. In some embodiments, a heavy chain variable region of the anti-B7H3 antibody or fragment thereof comprises or consists of the amino acid sequence of SEQ ID NO: 29, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof. In some embodiments, a heavy chain variable region of the anti-B7H3 antibody or fragment thereof comprises or consists of the amino acid sequence of SEQ ID NO: 29.

In some embodiments, a light chain variable region of the anti-B7H3 antibody or fragment thereof comprises or consists of the amino acid sequence of SEQ ID NO: 30 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof. In some embodiments, a light chain variable region of the anti-B7H3 antibody or fragment thereof comprises or consists of the amino acid sequence of SEQ ID NO: 30.

In some embodiments, a heavy chain variable region of the anti-B7H3 antibody or fragment thereof comprises or consists of the amino acid sequence of SEQ ID NO: 29, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, and a light chain variable region of the anti-B7H3 antibody or fragment thereof comprises or consists of the amino acid sequence of SEQ ID NO: 30 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof.

In some embodiments, a heavy chain variable region of the anti-B7H3 antibody or fragment thereof comprises or consists of the amino acid sequence of SEQ ID NO: 29, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the heavy chain variable region comprises: a HCDR1 comprising or consisting of the amino acid sequence of SEQ ID NO: 17; a HCDR2 comprising or consisting of the amino acid sequence of SEQ ID NO: 18; and a HCDR3 comprising or consisting of the amino acid sequence of SEQ ID NO: 19; and a light chain variable region of the anti-B7H3 antibody or fragment thereof comprises or consists of the amino acid sequence of SEQ ID NO: 30 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the light chain variable region comprises: a LCDR1 comprising or consisting of the amino acid sequence of SEQ ID NO: 20; a LCDR2 comprising or consisting of the amino acid sequence of SEQ ID NO: 21; and a LCDR3 comprising or consisting of the amino acid sequence of SEQ ID NO: 22.

In some embodiments, a heavy chain variable region of the anti-B7H3 antibody or fragment thereof comprises or consists of the amino acid sequence of SEQ ID NO: 29, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the heavy chain variable region comprises: a HCDR1 comprising or consisting of the amino acid sequence of SEQ ID NO: 23; a HCDR2 comprising or consisting of the amino acid sequence of SEQ ID NO: 24; and a HCDR3 comprising or consisting of the amino acid sequence of SEQ ID NO: 25; and a light chain variable region of the anti-B7H3 antibody or fragment thereof comprises or consists of the amino acid sequence of SEQ ID NO: 30 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the light chain variable region comprises: a LCDR1 comprising or consisting of the amino acid sequence of SEQ ID NO: 26; a LCDR2 comprising or consisting of the amino acid sequence of SEQ ID NO: 27; and a LCDR3 comprising or consisting of the amino acid sequence of SEQ ID NO: 28.

In some embodiments, a heavy chain variable region of the anti-B7H3 antibody or fragment thereof comprises or consists of the amino acid sequence of SEQ ID NO: 29; and a light chain variable region of the anti-B7H3 antibody or fragment thereof comprises or consists of the amino acid sequence of SEQ ID NO: 30. In some embodiments, a heavy chain variable region of the anti-B7H3 antibody or fragment thereof consists of the amino acid sequence of SEQ ID NO: 29; and a light chain variable region of the anti-B7H3 antibody or fragment thereof consists of the amino acid sequence of SEQ ID NO: 30.

In some embodiments, a heavy chain of the anti-B7H3 antibody or fragment thereof comprises or consists of the amino acid sequence of SEQ ID NO: 31, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof. In some embodiments, a heavy chain of the anti-B7H3 antibody or fragment thereof comprises or consists of the amino acid sequence of SEQ ID NO: 31.

In some embodiments, a light chain of the anti-B7H3 antibody or fragment thereof comprises or consists of the amino acid sequence of SEQ ID NO: 32 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof. In some embodiments, a light chain of the anti-B7H3 antibody or fragment thereof comprises or consists of the amino acid sequence of the amino acid sequence of SEQ ID NO: 32.

In some embodiments, a heavy chain of the anti-B7H3 antibody or fragment thereof comprises or consists of the amino acid sequence of the amino acid sequence of SEQ ID NO: 31, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof; and a light chain of the anti-B7H3 antibody or fragment thereof comprises or consists of the amino acid sequence of SEQ ID NO: 32 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof.

In some embodiments, a heavy chain of the anti-B7H3 antibody or fragment thereof comprises or consists of the amino acid sequence of SEQ ID NO: 31, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the heavy chain variable region comprises: a HCDR1 comprising or consisting of the amino acid sequence of SEQ ID NO: 17; a HCDR2 comprising or consisting of the amino acid sequence of SEQ ID NO: 18; and a HCDR3 comprising or consisting of the amino acid sequence of SEQ ID NO: 19; and a light chain of the anti-B7H3 antibody or fragment thereof comprises or consists of the amino acid sequence of SEQ ID NO: 32 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the light chain variable region comprises: a LCDR1 comprising or consisting of the amino acid sequence of SEQ ID NO: 20; a LCDR2 comprising or consisting of the amino acid sequence of SEQ ID NO: 21; and a LCDR3 comprising or consisting of the amino acid sequence of SEQ ID NO: 22.

In some embodiments, a heavy chain of the anti-B7H3 antibody or fragment thereof comprises or consists of the amino acid sequence of SEQ ID NO: 31, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the heavy chain variable region comprises: a HCDR1 comprising or consisting of the amino acid sequence of SEQ ID NO: 23; a HCDR2 comprising or consisting of the amino acid sequence of SEQ ID NO: 24; and a HCDR3 comprising or consisting of the amino acid sequence of SEQ ID NO: 25; and a light chain of the anti-B7H3 antibody or fragment thereof comprises or consists of the amino acid sequence of SEQ ID NO: 32 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the light chain variable region comprises: a LCDR1 comprising or consisting of the amino acid sequence of SEQ ID NO: 26; a LCDR2 comprising or consisting of the amino acid sequence of SEQ ID NO: 27; and a LCDR3 comprising or consisting of the amino acid sequence of SEQ ID NO: 28.

In some embodiments, a heavy chain of the anti-B7H3 antibody or fragment thereof comprises or consists of the amino acid sequence of SEQ ID NO: 31; and a light chain of the anti-B7H3 antibody or fragment thereof comprises or consists of the amino acid sequence of SEQ ID NO: 32. In some embodiments, a heavy chain of the anti-B7H3 antibody or fragment thereof consists of the amino acid sequence of SEQ ID NO: 31; and a light chain of the anti-B7H3 antibody or fragment thereof consists of the amino acid sequence of SEQ ID NO: 32.

In some embodiments, the anti-B7H3 antibody or fragment thereof comprises or consists of two heavy chains comprising or consisting of the amino acid sequence of SEQ ID NO: 31; and two light chain comprising or consisting of the amino acid sequence of SEQ ID NO: 32. In some embodiments, the anti-B7H3 antibody or fragment thereof comprises or consists of two heavy chains consisting of the amino acid sequence of SEQ ID NO: 31; and two light chains consisting of the amino acid sequence of SEQ ID NO: 32.

### Anti-TROP2 Antibodies and Fragments Thereof.

In some embodiments, the target expressed on the surface of a tumour cell is TROP2. TROP2 is a protein that in humans is encoded by the TACSTD2 and which may also be known as Tumour-associated calcium signal transducer 2, epithelial glycoprotein-1 antigen (EGP-1), or Trop-2.

In some embodiments, the antibody-drug conjugate comprises an anti-TROP2 antibody or fragment thereof.

The anti-TROP2 antibody or fragment thereof may specifically bind TROP2. The anti-TROP2 antibody may be a monoclonal antibody. In some embodiments, the anti-TROP2 antibody or fragment thereof is a humanized antibody. In some embodiments, the anti-TROP2 antibody or fragment thereof is a monoclonal humanized antibody. The fragment thereof may be any antigen-binding fragment thereof, for example a Fab, a Fab′, a F(ab′)2, a Fv, a scFv, a Fab′-SH, an sdAb, or a VHH. In some embodiments, the anti-TROP2 antibody or fragment thereof is a full-length anti-TROP2 antibody.

The anti-TROP2 antibody or fragment thereof may comprise a variable region that specifically binds TROP2. In some embodiments, the anti-TROP2 antibody or fragment thereof comprises a heavy chain variable region and/or a light chain variable domain. In some embodiments, the anti-TROP2 antibody or fragment thereof comprises a heavy chain variable region and a light chain variable domain. In some embodiments, the anti-TROP2 antibody or fragment thereof comprises a constant region, preferably derived from a human antibody, preferably the constant region is selected from the constant region of human IgG1, IgG2, IgG3 or IgG4. In some embodiments, the anti-TROP2 antibody or fragment thereof comprises a heavy chain and/or a light chain. In some embodiments, the antd-TROP2 antibody or fragment thereof comprises a heavy chain and a light chain. In some embodiments, the antd-TROP2 antibody or fragment thereof comprises or consists of two heavy chains and two light chains.

### Example Anti-TRP2 Antibody Sequences

Example anti-TROP2 antibody CDRs, variable region sequences, and heavy and light chain sequences are provided below.

| | |
|---|---|
| Kabat HCDR1 | NYGMN (SEQ ID NO: 33) |
| Kabat HCDR2 | WINTYTGEPTYTDDFKG (SEQ ID NO: 34) |
| Kabat HCDR3 | GGFGSSYWYFDV (SEQ ID NO: 35) |
| Kabat LCDR1 | KASQDVSIAVA (SEQ ID NO: 36) |
| Kabat LCDR2 | SASYRYT (SEQ ID NO: 37) |
| Kabat LCDR3 | QQHYITPLT (SEQ ID NO: 38) |
| IMGT HCDR1 | GYTFTNYG (SEQ ID NO: 39) |
| IMGT HCDR2 | INTYTGEP (SEQ ID NO: 40) |
| IMGT HCDR3 | ARGGFGSSYWYFDV (SEQ ID NO: 41) |
| IMGT LCDR1 | QDVSIA (SEQ ID NO: 42) |
| IMGT LCDR2 | SAS (SEQ ID NO: 43) |
| IMGT LCDR3 | QQHYITPLT (SEQ ID NO: 44) |
| VH | QVQLQQSGSELKKPGASVKVSCKASGYTFTNYGMNWVKQAPGQGL |
| | KWMGWINTYTGEPTYTDDFKGRFAFSLDTSVSTAYLQISSLKADDT |
| | AVYFCARGGFGSSYWYFDVWGQGSLVTVSS (SEQ ID NO: 45) |
| VL | DIQLTQSPSSLSASVGDRVSITCKASQDVSIAVAWYQQKPGKAPKLLI |
| | YSASYRYTGVPDRFSGSGSGTDFTLTISSLQPEDFAVYYCQQHYITPL |
| | TFGAGTKVEIK (SEQ ID NO: 46) |
| Heavy chain | QVQLQQSGSELKKPGASVKVSCKASGYTFTNYGMNWVKQAPGQGL |
| | KWMGWINTYTGEPTYTDDFKGRFAFSLDTSVSTAYLQISSLKADDT |
| | AVYFCARGGFGSSYWYFDVWGQGSLVTVSSASTKGPSVFPLAPSSK |
| | STSGGTAALGCLVKDYFPEPVTVSWNSGALTSGVHTFPAVLQSSGLY |
| | SLSSVVTVPSSSLGTQTYICNVNHKPSNTKVDKRVEPKSCDKTHTCPP |
| | CPAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYK |
| | CKVSNKALPAPIEKTISKAKGQPREPQVYTLPPSREEMTKNQVSLTCL |
| | VKGFYPSDIAVEWESNGQPENNYKTTPPVLDSDGSFFLYSKLTVDKS |
| | RWQQGNVFSCSVMHEALHNHYTQKSLSLSPGK (SEQ ID NO: 47) |
| Light chain | DIQLTQSPSSLSASVGDRVSITCKASQDVSIAVAWYQQKPGKAPKLLI |
| | YSASYRYTGVPDRFSGSGSGTDFTLTISSLQPEDFAVYYCQQHYITPL |
| | TFGAGTKVEIKRTVAAPSVFIFPPSDEQLKSGTASVVCLLNNFYPREA |
| | KVQWKVDNALQSGNSQESVTEQDSKDSTYSLSSTLTLSKADYEKHK |
| | VYACEVTHQGLSSPVTKSFNRGEC (SEQ ID NO: 48) |

In some embodiments, a heavy chain variable region of the anti-TROP2 antibody or fragment thereof comprises: (i) a HCDR1 comprising or consisting of the amino acid sequence of SEQ ID NO: 33, or a variant thereof having up to three amino acid substitutions, additions or deletions; (ii) a HCDR2 comprising or consisting of the amino acid sequence of SEQ ID NO: 34, or a variant thereof having up to three amino acid substitutions, additions or deletions; and (iii) a HCDR3 comprising or consisting of the amino acid sequence of SEQ ID NO: 35, or a variant thereof having up to three amino acid substitutions, additions or deletions, wherein the CDRs are defined according to the Kabat numbering system.

In some embodiments, a light chain variable region of the anti-TROP2 antibody or fragment thereof comprises: (i) a LCDR1 comprising or consisting of the amino acid sequence of SEQ ID NO: 36, or a variant thereof having up to three amino acid substitutions, additions or deletions; (ii) a LCDR2 comprising or consisting of the amino acid sequence of SEQ ID NO: 37, or a variant thereof having up to three amino acid substitutions, additions or deletions; and (iii) a LCDR3 comprising or consisting of the amino acid sequence of SEQ ID NO: 38, or a variant thereof having up to three amino acid substitutions, additions or deletions, wherein the CDRs are defined according to the Kabat numbering system.

In some embodiments, a heavy chain variable region of the anti-TROP2 antibody or fragment thereof comprises: (i) a HCDR1 comprising or consisting of the amino acid sequence of SEQ ID NO: 33, or a variant thereof having up to three amino acid substitutions, additions or deletions; (ii) a HCDR2 comprising or consisting of the amino acid sequence of SEQ ID NO: 34, or a variant thereof having up to three amino acid substitutions, additions or deletions; and (iii) a HCDR3 comprising or consisting of the amino acid sequence of SEQ ID NO: 35, or a variant thereof having up to three amino acid substitutions, additions or deletions; and a light chain variable region of the anti-TROP2 antibody or fragment thereof comprises: (i) a LCDR1 comprising or consisting of the amino acid sequence of SEQ ID NO: 36, or a variant thereof having up to three amino acid substitutions, additions or deletions; (ii) a LCDR2 comprising or consisting of the amino acid sequence of SEQ ID NO: 37, or a variant thereof having up to three amino acid substitutions, additions or deletions; and (iii) a LCDR3 comprising or consisting of the amino acid sequence of SEQ ID NO: 38, or a variant thereof having up to three amino acid substitutions, additions or deletions, wherein the CDRs are defined according to the Kabat numbering system.

In some embodiments, a heavy chain variable region of the anti-TROP2 antibody or fragment thereof comprises: (i) a HCDR1 comprising or consisting of the amino acid sequence of SEQ ID NO: 39, or a variant thereof having up to three amino acid substitutions, additions or deletions; (ii) a HCDR2 comprising or consisting of the amino acid sequence of SEQ ID NO: 40, or a variant thereof having up to three amino acid substitutions, additions or deletions; and (iii) a HCDR3 comprising or consisting of the amino acid sequence of SEQ ID NO: 41, or a variant thereof having up to three amino acid substitutions, additions or deletions, wherein the CDRs are defined according to the IMGT numbering system.

In some embodiments, a light chain variable region of the anti-TROP2 antibody or fragment thereof comprises: (i) a LCDR1 comprising or consisting of the amino acid sequence of SEQ ID NO: 42, or a variant thereof having up to three amino acid substitutions, additions or deletions; (ii) a LCDR2 comprising or consisting of the amino acid sequence of SEQ ID NO: 43; and (iii) a LCDR3 comprising or consisting of the amino acid sequence of SEQ ID NO: 44, or a variant thereof having up to three amino acid substitutions, additions or deletions, wherein the CDRs are defined according to the IMGT numbering system.

In some embodiments, a heavy chain variable region of the anti-TROP2 antibody or fragment thereof comprises: (i) a HCDR1 comprising or consisting of the amino acid sequence of SEQ ID NO: 39, or a variant thereof having up to three amino acid substitutions, additions or deletions; (ii) a HCDR2 comprising or consisting of the amino acid sequence of SEQ ID NO: 40, or a variant thereof having up to three amino acid substitutions, additions or deletions; and (iii) a HCDR3 comprising or consisting of the amino acid sequence of SEQ ID NO: 41, or a variant thereof having up to three amino acid substitutions, additions or deletions; and a light chain variable region of the anti-TROP2 antibody or fragment thereof comprises: (i) a LCDR1 comprising or consisting of the amino acid sequence of SEQ ID NO: 42, or a variant thereof having up to three amino acid substitutions, additions or deletions; (ii) a LCDR2 comprising or consisting of the amino acid sequence of SEQ ID NO: 43, or a variant thereof having up to three amino acid substitutions, additions or deletions; and (iii) a LCDR3 comprising or consisting of the amino acid sequence of SEQ ID NO: 44, or a variant thereof having up to three amino acid substitutions, additions or deletions, wherein the CDRs are defined according to the IMGT numbering system.

In some embodiments, the CDR variants have up to two amino acid substitutions, additions or deletions. In some embodiments, the CDR variants have up to one amino acid substitution, addition or deletion. In some embodiments, the CDR variants have up to three amino acid substitutions. In some embodiments, the CDR variants have up to two amino acid substitutions. In some embodiments, the CDR variants have up to one amino acid substitution.

In some embodiments, a heavy chain variable region of the anti-TROP2 antibody or fragment thereof comprises or consists of the amino acid sequence of SEQ ID NO: 45, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof. In some embodiments, a heavy chain variable region of the anti-TROP2 antibody or fragment thereof comprises or consists of the amino acid sequence of SEQ ID NO: 45.

In some embodiments, a light chain variable region of the anti-TROP2 antibody or fragment thereof comprises or consists of the amino acid sequence of SEQ ID NO: 46 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof. In some embodiments, a light chain variable region of the anti-TROP2 antibody or fragment thereof comprises or consists of the amino acid sequence of SEQ ID NO: 46.

In some embodiments, a heavy chain variable region of the anti-TROP2 antibody or fragment thereof comprises or consists of the amino acid sequence of SEQ ID NO: 45, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, and a light chain variable region of the anti-TROP2 antibody or fragment thereof comprises or consists of the amino acid sequence of SEQ ID NO: 46 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof.

In some embodiments, a heavy chain variable region of the anti-TROP2 antibody or fragment thereof comprises or consists of the amino acid sequence of SEQ ID NO: 45, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the heavy chain variable region comprises: a HCDR1 comprising or consisting of the amino acid sequence of SEQ ID NO: 33; a HCDR2 comprising or consisting of the amino acid sequence of SEQ ID NO: 34; and a HCDR3 comprising or consisting of the amino acid sequence of SEQ ID NO: 35; and a light chain variable region of the anti-TROP2 antibody or fragment thereof comprises or consists of the amino acid sequence of SEQ ID NO: 46 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the light chain variable region comprises: a LCDR1 comprising or consisting of the amino acid sequence of SEQ ID NO: 36; a LCDR2 comprising or consisting of the amino acid sequence of SEQ ID NO: 37; and a LCDR3 comprising or consisting of the amino acid sequence of SEQ ID NO: 38.

In some embodiments, a heavy chain variable region of the anti-TROP2 antibody or fragment thereof comprises or consists of the amino acid sequence of SEQ ID NO: 45, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the heavy chain variable region comprises: a HCDR1 comprising or consisting of the amino acid sequence of SEQ ID NO: 39; a HCDR2 comprising or consisting of the amino acid sequence of SEQ ID NO: 40; and a HCDR3 comprising or consisting of the amino acid sequence of SEQ ID NO: 41; and a light chain variable region of the anti-TROP2 antibody or fragment thereof comprises or consists of the amino acid sequence of SEQ ID NO: 46 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the light chain variable region comprises: a LCDR1 comprising or consisting of the amino acid sequence of SEQ ID NO: 42; a LCDR2 comprising or consisting of the amino acid sequence of SEQ ID NO: 43; and a LCDR3 comprising or consisting of the amino acid sequence of SEQ ID NO: 44.

In some embodiments, a heavy chain variable region of the anti-TROP2 antibody or fragment thereof comprises or consists of the amino acid sequence of SEQ ID NO: 45; and a light chain variable region of the anti-TROP2 antibody or fragment thereof comprises or consists of the amino acid sequence of SEQ ID NO: 46. In some embodiments, a heavy chain variable region of the anti-TROP2 antibody or fragment thereof consists of the amino acid sequence of SEQ ID NO: 45; and a light chain variable region of the anti-TROP2 antibody or fragment thereof consists of the amino acid sequence of SEQ ID NO: 46.

In some embodiments, a heavy chain of the anti-TROP2 antibody or fragment thereof comprises or consists of the amino acid sequence of SEQ ID NO: 47, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof. In some embodiments, a heavy chain of the anti-TROP2 antibody or fragment thereof comprises or consists of the amino acid sequence of SEQ ID NO: 47.

In some embodiments, a light chain of the anti-TROP2 antibody or fragment thereof comprises or consists of the amino acid sequence of SEQ ID NO: 48 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof. In some embodiments, a light chain of the anti-TROP2 antibody or fragment thereof comprises or consists of the amino acid sequence of the amino acid sequence of SEQ ID NO: 48.

In some embodiments, a heavy chain of the anti-TROP2 antibody or fragment thereof comprises or consists of the amino acid sequence of the amino acid sequence of SEQ ID NO: 47, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof; and a light chain of the anti-TROP2 antibody or fragment thereof comprises or consists of the amino acid sequence of SEQ ID NO: 48 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof.

In some embodiments, a heavy chain of the anti-TROP2 antibody or fragment thereof comprises or consists of the amino acid sequence of SEQ ID NO: 47, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the heavy chain variable region comprises: a HCDR1 comprising or consisting of the amino acid sequence of SEQ ID NO: 33; a HCDR2 comprising or consisting of the amino acid sequence of SEQ ID NO: 34; and a HCDR3 comprising or consisting of the amino acid sequence of SEQ ID NO: 35; and a light chain of the anti-TROP2 antibody or fragment thereof comprises or consists of the amino acid sequence of SEQ ID NO: 48 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the light chain variable region comprises: a LCDR1 comprising or consisting of the amino acid sequence of SEQ ID NO: 36; a LCDR2 comprising or consisting of the amino acid sequence of SEQ ID NO: 37; and a LCDR3 comprising or consisting of the amino acid sequence of SEQ ID NO: 38.

In some embodiments, a heavy chain of the anti-TROP2 antibody or fragment thereof comprises or consists of the amino acid sequence of SEQ ID NO: 47, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the heavy chain variable region comprises: a HCDR1 comprising or consisting of the amino acid sequence of SEQ ID NO: 39; a HCDR2 comprising or consisting of the amino acid sequence of SEQ ID NO: 40; and a HCDR3 comprising or consisting of the amino acid sequence of SEQ ID NO: 41; and a light chain of the anti-TROP2 antibody or fragment thereof comprises or consists of the amino acid sequence of SEQ ID NO: 48 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the light chain variable region comprises: a LCDR1 comprising or consisting of the amino acid sequence of SEQ ID NO: 42; a LCDR2 comprising or consisting of the amino acid sequence of SEQ ID NO: 43; and a LCDR3 comprising or consisting of the amino acid sequence of SEQ ID NO: 44.

In some embodiments, a heavy chain of the anti-TROP2 antibody or fragment thereof comprises or consists of the amino acid sequence of SEQ ID NO: 47; and a light chain of the anti-TROP2 antibody or fragment thereof comprises or consists of the amino acid sequence of SEQ ID NO: 48. In some embodiments, a heavy chain of the anti-TROP2 antibody or fragment thereof consists of the amino acid sequence of SEQ ID NO: 47; and a light chain of the anti-TROP2 antibody or fragment thereof consists of the amino acid sequence of SEQ ID NO: 48.

In some embodiments, the anti-TROP2 antibody or fragment thereof comprises or consists of two heavy chains comprising or consisting of the amino acid sequence of SEQ ID NO: 47; and two light chain comprising or consisting of the amino acid sequence of SEQ ID NO: 48. In some embodiments, the anti-TROP2 antibody or fragment thereof comprises or consists of two heavy chains consisting of the amino acid sequence of SEQ ID NO: 47; and two light chains consisting of the amino acid sequence of SEQ ID NO: 48.

In some embodiments, the anti-TROP2 antibody or fragment thereof is Sacituzumab or a fragment thereof. In some embodiments, the anti-TROP2 antibody or fragment thereof is Sacituzumab.

### Anti-HER2 Antibodies and Fragments Thereof.

In some embodiments, the target expressed on the surface of a tumour cell is HER2. HER2 is a protein that in humans is encoded by the ERBB2 gene and which may also be known as human epidermal growth factor receptor 2 (HER-2), Receptor tyrosine-protein kinase erbB-2 and CD340 (Cluster of Differentiation 340).

In some embodiments, the antibody-drug conjugate comprises an anti-HER2 antibody or fragment thereof.

The anti-HER2 antibody or fragment thereof may specifically bind HER2. The anti-HER2 antibody may be a monoclonal antibody. In some embodiments, the anti-HER2 antibody or fragment thereof is a humanized antibody. In some embodiments, the anti-HER2 antibody or fragment thereof is a monoclonal humanized antibody. The fragment thereof may be any antigen-binding fragment thereof, for example a Fab, a Fab′, a F(ab′)2, a Fv, an ScFv, a Fab′-SH, an sdAb, or a VHH. In some embodiments, the anti-HER2 antibody or fragment thereof is a full-length anti-HER2 antibody.

The anti-HER2 antibody or fragment thereof may comprise a variable region that specifically binds HER2. In some embodiments, the anti-HER2 antibody or fragment thereof comprises a heavy chain variable region and/or a light chain variable domain. In some embodiments, the anti-HER2 antibody or fragment thereof comprises a heavy chain variable region and a light chain variable domain. In some embodiments, the anti-HER2 antibody or fragment thereof comprises a constant region, preferably derived from a human antibody, preferably the constant region is selected from the constant region of human IgG1, IgG2, IgG3 or IgG4. In some embodiments, the anti-HER2 antibody or fragment thereof comprises a heavy chain and/or a light chain. In some embodiments, the anti-HER2 antibody or fragment thereof comprises a heavy chain and a light chain. In some embodiments, the anti-HER2 antibody or fragment thereof comprises or consists of two heavy chains and two light chains.

Example anti-HER2 antibody CDRs, variable region sequences, and heavy and light chain sequences are provided below.

### Example Anti-HER2 Antibody Sequences 1—Trastuzumab

| | |
|---|---|
| Kabat HCDR1 | DTYIH (SEQ ID NO: 49) |
| Kabat HCDR2 | RIYPTNGYTRYADSVKG (SEQ ID NO: 50) |
| Kabat HCDR3 | WGGDGFYAMDY (SEQ ID NO: 51) |
| Kabat LCDR1 | RASQDVNTAVA (SEQ ID NO: 52) |
| Kabat LCDR2 | SASFLYS (SEQ ID NO: 53) |
| Kabat LCDR3 | QQHYTTPPT (SEQ ID NO: 54) |
| IMGT HCDR1 | GFNIKDTY (SEQ ID NO: 55) |
| IMGT HCDR2 | IYPTNGYT (SEQ ID NO: 56) |
| IMGT HCDR3 | SRWGGDGFY AMDY (SEQ ID NO: 57) |
| IMGT LCDR1 | QDVNTA (SEQ ID NO: 58) |
| IMGT LCDR2 | SAS (SEQ ID NO: 59) |
| IMGT LCDR3 | QQHYTTPPT (SEQ ID NO: 60) |
| VH | EVQLVESGGGLVQPGGSLRLSCAASGFNIKDTYIHWVRQAPGKGLE |
| | WVARIYPTNGYTRYADSVKGRFTISADTSKNTAYLQMNSLRAEDTA |
| | VYYCSRWGGDGFYAMDYWGQGTLVTVSS (SEQ ID NO: 61) |
| VL | DIQMTQSPSSLSASVGDRVTITCRASQDVNTAVAWYQQKPGKAPKL |
| | LIYSASFLYSGVPSRFSGSRSGTDFTLTISSLQPEDFATYYCQQHYTTP |
| | PTFGQGTKVEIK (SEQ ID NO: 62) |
| Heavy chain | EVQLVESGGGLVQPGGSLRLSCAASGFNIKDTYIHWVRQAPGKGLE |
| | WVARIYPTNGYTRYADSVKGRFTISADTSKNTAYLQMNSLRAEDTA |
| | VYYCSRWGGDGFY AMDYWGQGTLVTVSSASTKGPSVFPLAPSSKST |
| | SGGTAALGCLVKDYFPEPVTVSWNSGALTSGVHTFPAVLQSSGLYSL |
| | SSVVTVPSSSLGTQTYICNVNHKPSNTKVDKKVEPKSCDKTHTCPPC |
| | PAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYK |
| | CKVSNKALPAPIEKTISKAKGQPREPQVYTLPPSREEMTKNQVSLTCL |
| | VKGFYPSDIAVEWESNGQPENNYKTTPPVLDSDGSFFLYSKLTVDKS |
| | RWQQGNVFSCSVMHEALHNHYTQKSLSLSPG (SEQ ID NO: 63) |
| Light chain | DIQMTQSPSSLSASVGDRVTITCRASQDVNTAVAWYQQKPGKAPKL |
| | LIYSASFLYSGVPSRFSGSRSGTDFTLTISSLQPEDFATYYCQQHYTTP |
| | PTFGQGTKVEIKRTVAAPSVFIFPPSDEQLKSGTASVVCLLNNFYPRE |
| | AKVQWKVDNALQSGNSQESVTEQDSKDSTYSLSSTLTLSKADYEKH |
| | KVYACEVTHQGLSSPVTKSFNRGEC (SEQ ID NO: 64) |

In some embodiments, a heavy chain variable region of the anti-HER2 antibody or fragment thereof comprises: (i) a HCDR1 comprising or consisting of the amino acid sequence of SEQ ID NO: 49, or a variant thereof having up to three amino acid substitutions, additions or deletions; (ii) a HCDR2 comprising or consisting of the amino acid sequence of SEQ ID NO: 50, or a variant thereof having up to three amino acid substitutions, additions or deletions; and (iii) a HCDR3 comprising or consisting of the amino acid sequence of SEQ ID NO: 51, or a variant thereof having up to three amino acid substitutions, additions or deletions, wherein the CDRs are defined according to the Kabat numbering system.

In some embodiments, a light chain variable region of the anti-HER2 antibody or fragment thereof comprises: (i) a LCDR1 comprising or consisting of the amino acid sequence of SEQ ID NO: 52, or a variant thereof having up to three amino acid substitutions, additions or deletions; (ii) a LCDR2 comprising or consisting of the amino acid sequence of SEQ ID NO: 53, or a variant thereof having up to three amino acid substitutions, additions or deletions; and (iii) a LCDR3 comprising or consisting of the amino acid sequence of SEQ ID NO: 54, or a variant thereof having up to three amino acid substitutions, additions or deletions, wherein the CDRs are defined according to the Kabat numbering system.

In some embodiments, a heavy chain variable region of the anti-HER2 antibody or fragment thereof comprises: (i) a HCDR1 comprising or consisting of the amino acid sequence of SEQ ID NO: 49, or a variant thereof having up to three amino acid substitutions, additions or deletions; (ii) a HCDR2 comprising or consisting of the amino acid sequence of SEQ ID NO: 50, or a variant thereof having up to three amino acid substitutions, additions or deletions; and (iii) a HCDR3 comprising or consisting of the amino acid sequence of SEQ ID NO: 51, or a variant thereof having up to three amino acid substitutions, additions or deletions; and a light chain variable region of the anti-HER2 antibody or fragment thereof comprises: (i) a LCDR1 comprising or consisting of the amino acid sequence of SEQ ID NO: 52, or a variant thereof having up to three amino acid substitutions, additions or deletions; (ii) a LCDR2 comprising or consisting of the amino acid sequence of SEQ ID NO: 53, or a variant thereof having up to three amino acid substitutions, additions or deletions; and (iii) a LCDR3 comprising or consisting of the amino acid sequence of SEQ ID NO: 54, or a variant thereof having up to three amino acid substitutions, additions or deletions, wherein the CDRs are defined according to the Kabat numbering system.

In some embodiments, a heavy chain variable region of the anti-HER2 antibody or fragment thereof comprises: (i) a HCDR1 comprising or consisting of the amino acid sequence of SEQ ID NO: 55, or a variant thereof having up to three amino acid substitutions, additions or deletions; (ii) a HCDR2 comprising or consisting of the amino acid sequence of SEQ ID NO: 56, or a variant thereof having up to three amino acid substitutions, additions or deletions; and (iii) a HCDR3 comprising or consisting of the amino acid sequence of SEQ ID NO: 57, or a variant thereof having up to three amino acid substitutions, additions or deletions, wherein the CDRs are defined according to the IMGT numbering system.

In some embodiments, a light chain variable region of the anti-HER2 antibody or fragment thereof comprises: (i) a LCDR1 comprising or consisting of the amino acid sequence of SEQ ID NO: 58, or a variant thereof having up to three amino acid substitutions, additions or deletions; (ii) a LCDR2 comprising or consisting of the amino acid sequence of SEQ ID NO: 59; and (iii) a LCDR3 comprising or consisting of the amino acid sequence of SEQ ID NO: 60, or a variant thereof having up to three amino acid substitutions, additions or deletions, wherein the CDRs are defined according to the IMGT numbering system.

In some embodiments, a heavy chain variable region of the anti-HER2 antibody or fragment thereof comprises: (i) a HCDR1 comprising or consisting of the amino acid sequence of SEQ ID NO: 55, or a variant thereof having up to three amino acid substitutions, additions or deletions; (ii) a HCDR2 comprising or consisting of the amino acid sequence of SEQ ID NO: 56, or a variant thereof having up to three amino acid substitutions, additions or deletions; and (iii) a HCDR3 comprising or consisting of the amino acid sequence of SEQ ID NO: 57, or a variant thereof having up to three amino acid substitutions, additions or deletions; and a light chain variable region of the anti-HER2 antibody or fragment thereof comprises: (i) a LCDR1 comprising or consisting of the amino acid sequence of SEQ ID NO: 58, or a variant thereof having up to three amino acid substitutions, additions or deletions; (ii) a LCDR2 comprising or consisting of the amino acid sequence of SEQ ID NO: 59, or a variant thereof having up to three amino acid substitutions, additions or deletions; and (iii) a LCDR3 comprising or consisting of the amino acid sequence of SEQ ID NO: 60, or a variant thereof having up to three amino acid substitutions, additions or deletions, wherein the CDRs are defined according to the IMGT numbering system.

In some embodiments, the CDR variants have up to two amino acid substitutions, additions or deletions. In some embodiments, the CDR variants have up to one amino acid substitution, addition or deletion. In some embodiments, the CDR variants have up to three amino acid substitutions. In some embodiments, the CDR variants have up to two amino acid substitutions. In some embodiments, the CDR variants have up to one amino acid substitution.

In some embodiments, a heavy chain variable region of the anti-HER2 antibody or fragment thereof comprises or consists of the amino acid sequence of SEQ ID NO: 61, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof. In some embodiments, a heavy chain variable region of the anti-HER2 antibody or fragment thereof comprises or consists of the amino acid sequence of SEQ ID NO: 61.

In some embodiments, a light chain variable region of the anti-HER2 antibody or fragment thereof comprises or consists of the amino acid sequence of SEQ ID NO: 62 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof. In some embodiments, a light chain variable region of the anti-HER2 antibody or fragment thereof comprises or consists of the amino acid sequence of SEQ ID NO: 62.

In some embodiments, a heavy chain variable region of the anti-HER2 antibody or fragment thereof comprises or consists of the amino acid sequence of SEQ ID NO: 61, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, and a light chain variable region of the anti-HER2 antibody or fragment thereof comprises or consists of the amino acid sequence of SEQ ID NO: 62 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof.

In some embodiments, a heavy chain variable region of the anti-HER2 antibody or fragment thereof comprises or consists of the amino acid sequence of SEQ ID NO: 61, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the heavy chain variable region comprises: a HCDR1 comprising or consisting of the amino acid sequence of SEQ ID NO: 49; a HCDR2 comprising or consisting of the amino acid sequence of SEQ ID NO: 50; and a HCDR3 comprising or consisting of the amino acid sequence of SEQ ID NO: 51; and a light chain variable region of the anti-HER2 antibody or fragment thereof comprises or consists of the amino acid sequence of SEQ ID NO: 62 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the light chain variable region comprises: a LCDR1 comprising or consisting of the amino acid sequence of SEQ ID NO: 52; a LCDR2 comprising or consisting of the amino acid sequence of SEQ ID NO: 53; and a LCDR3 comprising or consisting of the amino acid sequence of SEQ ID NO: 54.

In some embodiments, a heavy chain variable region of the anti-HER2 antibody or fragment thereof comprises or consists of the amino acid sequence of SEQ ID NO: 61, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the heavy chain variable region comprises: a HCDR1 comprising or consisting of the amino acid sequence of SEQ ID NO: 55; a HCDR2 comprising or consisting of the amino acid sequence of SEQ ID NO: 56; and a HCDR3 comprising or consisting of the amino acid sequence of SEQ ID NO: 57; and a light chain variable region of the anti-HER2 antibody or fragment thereof comprises or consists of the amino acid sequence of SEQ ID NO: 62 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the light chain variable region comprises: a LCDR1 comprising or consisting of the amino acid sequence of SEQ ID NO: 58; a LCDR2 comprising or consisting of the amino acid sequence of SEQ ID NO: 59; and a LCDR3 comprising or consisting of the amino acid sequence of SEQ ID NO: 60.

In some embodiments, a heavy chain variable region of the anti-HER2 antibody or fragment thereof comprises or consists of the amino acid sequence of SEQ ID NO: 61; and a light chain variable region of the anti-HER2 antibody or fragment thereof comprises or consists of the amino acid sequence of SEQ ID NO: 62. In some embodiments, a heavy chain variable region of the anti-HER2 antibody or fragment thereof consists of the amino acid sequence of SEQ ID NO: 61; and a light chain variable region of the anti-HER2 antibody or fragment thereof consists of the amino acid sequence of SEQ ID NO: 62.

In some embodiments, a heavy chain of the anti-HER2 antibody or fragment thereof comprises or consists of the amino acid sequence of SEQ ID NO: 63, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof. In some embodiments, a heavy chain of the anti-HER2 antibody or fragment thereof comprises or consists of the amino acid sequence of SEQ ID NO: 63.

In some embodiments, a light chain of the anti-HER2 antibody or fragment thereof comprises or consists of the amino acid sequence of SEQ ID NO: 64 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof. In some embodiments, a light chain of the anti-HER2 antibody or fragment thereof comprises or consists of the amino acid sequence of the amino acid sequence of SEQ ID NO: 64.

In some embodiments, a heavy chain of the anti-HER2 antibody or fragment thereof comprises or consists of the amino acid sequence of the amino acid sequence of SEQ ID NO: 63, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof; and a light chain of the anti-HER2 antibody or fragment thereof comprises or consists of the amino acid sequence of SEQ ID NO: 64 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof.

In some embodiments, a heavy chain of the anti-HER2 antibody or fragment thereof comprises or consists of the amino acid sequence of SEQ ID NO: 63, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the heavy chain variable region comprises: a HCDR1 comprising or consisting of the amino acid sequence of SEQ ID NO: 49; a HCDR2 comprising or consisting of the amino acid sequence of SEQ ID NO: 50; and a HCDR3 comprising or consisting of the amino acid sequence of SEQ ID NO: 51; and a light chain of the anti-HER2 antibody or fragment thereof comprises or consists of the amino acid sequence of SEQ ID NO: 64 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the light chain variable region comprises: a LCDR1 comprising or consisting of the amino acid sequence of SEQ ID NO: 52; a LCDR2 comprising or consisting of the amino acid sequence of SEQ ID NO: 53; and a LCDR3 comprising or consisting of the amino acid sequence of SEQ ID NO: 54.

In some embodiments, a heavy chain of the anti-HER2 antibody or fragment thereof comprises or consists of the amino acid sequence of SEQ ID NO: 63, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the heavy chain variable region comprises: a HCDR1 comprising or consisting of the amino acid sequence of SEQ ID NO: 55; a HCDR2 comprising or consisting of the amino acid sequence of SEQ ID NO: 56; and a HCDR3 comprising or consisting of the amino acid sequence of SEQ ID NO: 57; and a light chain of the anti-HER2 antibody or fragment thereof comprises or consists of the amino acid sequence of SEQ ID NO: 64 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the light chain variable region comprises: a LCDR1 comprising or consisting of the amino acid sequence of SEQ ID NO: 58; a LCDR2 comprising or consisting of the amino acid sequence of SEQ ID NO: 59; and a LCDR3 comprising or consisting of the amino acid sequence of SEQ ID NO: 60.

In some embodiments, a heavy chain of the anti-HER2 antibody or fragment thereof comprises or consists of the amino acid sequence of SEQ ID NO: 63; and a light chain of the anti-HER2 antibody or fragment thereof comprises or consists of the amino acid sequence of SEQ ID NO: 64. In some embodiments, a heavy chain of the anti-HER2 antibody or fragment thereof consists of the amino acid sequence of SEQ ID NO: 63; and a light chain of the anti-HER2 antibody or fragment thereof consists of the amino acid sequence of SEQ ID NO: 64.

In some embodiments, the anti-HER2 antibody or fragment thereof comprises or consists of two heavy chains comprising or consisting of the amino acid sequence of SEQ ID NO: 63; and two light chain comprising or consisting of the amino acid sequence of SEQ ID NO: 64. In some embodiments, the anti-HER2 antibody or fragment thereof comprises or consists of two heavy chains consisting of the amino acid sequence of SEQ ID NO: 63; and two light chains consisting of the amino acid sequence of SEQ ID NO: 64.

In some embodiments, the anti-HER2 antibody or fragment thereof is Trastuzumab or a fragment thereof. In some embodiments, the anti-HER2 antibody or fragment thereof is Trastuzumab.

### Example Anti-HER2 Antibody Sequences 2—Pertuzumab

| | |
|---|---|
| Kabat HCDR1 | DYTMD (SEQ ID NO: 65) |
| Kabat HCDR2 | DVNPNSGGSIYNQRFKG (SEQ ID NO: 66) |
| Kabat HCDR3 | NLGPSFYFDY (SEQ ID NO: 67) |
| Kabat LCDR1 | KASQDVSIGVA (SEQ ID NO: 68) |
| Kabat LCDR2 | SASYRYT (SEQ ID NO: 69) |
| Kabat LCDR3 | QQYYIYPYT (SEQ ID NO: 70) |
| IMGT HCDR1 | GFTFTDYT (SEQ ID NO: 71) |
| IMGT HCDR2 | VNPNSGGS (SEQ ID NO: 72) |
| IMGT HCDR3 | ARNLGPSFYFDY (SEQ ID NO: 73) |
| IMGT LCDR1 | QDVSIG (SEQ ID NO: 74) |
| IMGT LCDR2 | SAS (SEQ ID NO: 75) |
| IMGT LCDR3 | QQYYIYPYT (SEQ ID NO: 76) |
| VH | EVQLVESGGGLVQPGGSLRLSCAASGFTFTDYTMDWVRQAPGKGLE |
| | WVADVNPNSGGSIYNQRFKGRFTLSVDRSKNTLYLQMNSLRAEDTA |
| | VYYCARNLGPSFYFDYWGQGTLVTVSS (SEQ ID NO: 77) |
| VL | DIQMTQSPSSLSASVGDRVTITCKASQDVSIGVAWYQQKPGKAPKLL |
| | IYSASYRYTGVPSRFSGSGSGTDFTLTISSLQPEDFATYYCQQYYIY |
| | PYTFGQGTKVEIK (SEQ ID NO: 78) |
| Heavy chain | EVQLVESGGGLVQPGGSLRLSCAASGFTFTDYTMDWVRQAPGKGLE |
| | WVADVNPNSGGSIYNQRFKGRFTLSVDRSKNTLYLQMNSLRAEDTA |
| | VYYCARNLGPSFYFDYWGQGTLVTVSSASTKGPSVFPLAPSSKSTSG |
| | GTAALGCLVKDYFPEPVTVSWNSGALTSGVHTFPAVLQSSGLYSLSS |
| | VVTVPSSSLGTQTYICNVNHKPSNTKVDKKVEPKSCDKTHTCPPCPA |
| | PELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKVS |
| | NKALPAPIEKTISKAKGQPREPQVYTLPPSREEMTKNQVSLTCLVKG |
| | FYPSDIAVEWESNGQPENNYKTTPPVLDSDGSFFLYSKLTVDKSRWQ |
| | QGNVFSCSVMHEALHNHYTQKSLSLSPG (SEQ ID NO: 79) |
| Light chain | DIQMTQSPSSLSASVGDRVTITCKASQDVSIGVAWYQQKPGKAPKLL |
| | IYSASYRYTGVPSRFSGSGSGTDFTLTISSLQPEDFATYYCQQYYIYPY |
| | TFGQGTKVEIKRTVAAPSVFIFPPSDEQLKSGTASVVCLLNNFYPREA |
| | KVQWKVDNALQSGNSQESVTEQDSKDSTYSLSSTLTLSKADYEKHK |
| | VYACEVTHQGLSSPVTKSFNRGEC (SEQ ID NO: 80) |

In some embodiments, a heavy chain variable region of the anti-HER2 antibody or fragment thereof comprises: (i) a HCDR1 comprising or consisting of the amino acid sequence of SEQ ID NO: 65, or a variant thereof having up to three amino acid substitutions, additions or deletions; (ii) a HCDR2 comprising or consisting of the amino acid sequence of SEQ ID NO: 66, or a variant thereof having up to three amino acid substitutions, additions or deletions; and (iii) a HCDR3 comprising or consisting of the amino acid sequence of SEQ ID NO: 67, or a variant thereof having up to three amino acid substitutions, additions or deletions, wherein the CDRs are defined according to the Kabat numbering system.

In some embodiments, a light chain variable region of the anti-HER2 antibody or fragment thereof comprises: (i) a LCDR1 comprising or consisting of the amino acid sequence of SEQ ID NO: 68, or a variant thereof having up to three amino acid substitutions, additions or deletions; (ii) a LCDR2 comprising or consisting of the amino acid sequence of SEQ ID NO: 69, or a variant thereof having up to three amino acid substitutions, additions or deletions; and (iii) a LCDR3 comprising or consisting of the amino acid sequence of SEQ ID NO: 70, or a variant thereof having up to three amino acid substitutions, additions or deletions, wherein the CDRs are defined according to the Kabat numbering system.

In some embodiments, a heavy chain variable region of the anti-HER2 antibody or fragment thereof comprises: (i) a HCDR1 comprising or consisting of the amino acid sequence of SEQ ID NO: 65, or a variant thereof having up to three amino acid substitutions, additions or deletions; (ii) a HCDR2 comprising or consisting of the amino acid sequence of SEQ ID NO: 66, or a variant thereof having up to three amino acid substitutions, additions or deletions; and (iii) a HCDR3 comprising or consisting of the amino acid sequence of SEQ ID NO: 67, or a variant thereof having up to three amino acid substitutions, additions or deletions; and a light chain variable region of the anti-HER2 antibody or fragment thereof comprises: (i) a LCDR1 comprising or consisting of the amino acid sequence of SEQ ID NO: 68, or a variant thereof having up to three amino acid substitutions, additions or deletions; (ii) a LCDR2 comprising or consisting of the amino acid sequence of SEQ ID NO: 69, or a variant thereof having up to three amino acid substitutions, additions or deletions; and (iii) a LCDR3 comprising or consisting of the amino acid sequence of SEQ ID NO: 70, or a variant thereof having up to three amino acid substitutions, additions or deletions, wherein the CDRs are defined according to the Kabat numbering system.

In some embodiments, a heavy chain variable region of the anti-HER2 antibody or fragment thereof comprises: (i) a HCDR1 comprising or consisting of the amino acid sequence of SEQ ID NO: 71, or a variant thereof having up to three amino acid substitutions, additions or deletions; (ii) a HCDR2 comprising or consisting of the amino acid sequence of SEQ ID NO: 72, or a variant thereof having up to three amino acid substitutions, additions or deletions; and (iii) a HCDR3 comprising or consisting of the amino acid sequence of SEQ ID NO: 73, or a variant thereof having up to three amino acid substitutions, additions or deletions, wherein the CDRs are defined according to the IMGT numbering system.

In some embodiments, a light chain variable region of the anti-HER2 antibody or fragment thereof comprises: (i) a LCDR1 comprising or consisting of the amino acid sequence of SEQ ID NO: 74, or a variant thereof having up to three amino acid substitutions, additions or deletions; (ii) a LCDR2 comprising or consisting of the amino acid sequence of SEQ ID NO: 75; and (iii) a LCDR3 comprising or consisting of the amino acid sequence of SEQ ID NO: 76, or a variant thereof having up to three amino acid substitutions, additions or deletions, wherein the CDRs are defined according to the IMGT numbering system.

In some embodiments, a heavy chain variable region of the anti-HER2 antibody or fragment thereof comprises: (i) a HCDR1 comprising or consisting of the amino acid sequence of SEQ ID NO: 71, or a variant thereof having up to three amino acid substitutions, additions or deletions; (ii) a HCDR2 comprising or consisting of the amino acid sequence of SEQ ID NO: 72, or a variant thereof having up to three amino acid substitutions, additions or deletions; and (iii) a HCDR3 comprising or consisting of the amino acid sequence of SEQ ID NO: 73, or a variant thereof having up to three amino acid substitutions, additions or deletions; and a light chain variable region of the anti-HER2 antibody or fragment thereof comprises: (i) a LCDR1 comprising or consisting of the amino acid sequence of SEQ ID NO: 74, or a variant thereof having up to three amino acid substitutions, additions or deletions; (ii) a LCDR2 comprising or consisting of the amino acid sequence of SEQ ID NO: 75, or a variant thereof having up to three amino acid substitutions, additions or deletions; and (iii) a LCDR3 comprising or consisting of the amino acid sequence of SEQ ID NO: 76, or a variant thereof having up to three amino acid substitutions, additions or deletions, wherein the CDRs are defined according to the IMGT numbering system.

In some embodiments, the CDR variants have up to two amino acid substitutions, additions or deletions. In some embodiments, the CDR variants have up to one amino acid substitution, addition or deletion. In some embodiments, the CDR variants have up to three amino acid substitutions. In some embodiments, the CDR variants have up to two amino acid substitutions. In some embodiments, the CDR variants have up to one amino acid substitution.

In some embodiments, a heavy chain variable region of the anti-HER2 antibody or fragment thereof comprises or consists of the amino acid sequence of SEQ ID NO: 77, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof. In some embodiments, a heavy chain variable region of the anti-HER2 antibody or fragment thereof comprises or consists of the amino acid sequence of SEQ ID NO: 77.

In some embodiments, a light chain variable region of the anti-HER2 antibody or fragment thereof comprises or consists of the amino acid sequence of SEQ ID NO: 78 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof. In some embodiments, a light chain variable region of the anti-HER2 antibody or fragment thereof comprises or consists of the amino acid sequence of SEQ ID NO: 78.

In some embodiments, a heavy chain variable region of the anti-HER2 antibody or fragment thereof comprises or consists of the amino acid sequence of SEQ ID NO: 77, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, and a light chain variable region of the anti-HER2 antibody or fragment thereof comprises or consists of the amino acid sequence of SEQ ID NO: 78 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof.

In some embodiments, a heavy chain variable region of the anti-HER2 antibody or fragment thereof comprises or consists of the amino acid sequence of SEQ ID NO: 77, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the heavy chain variable region comprises: a HCDR1 comprising or consisting of the amino acid sequence of SEQ ID NO: 65; a HCDR2 comprising or consisting of the amino acid sequence of SEQ ID NO: 66; and a HCDR3 comprising or consisting of the amino acid sequence of SEQ ID NO: 67; and a light chain variable region of the anti-HER2 antibody or fragment thereof comprises or consists of the amino acid sequence of SEQ ID NO: 78 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the light chain variable region comprises: a LCDR1 comprising or consisting of the amino acid sequence of SEQ ID NO: 68; a LCDR2 comprising or consisting of the amino acid sequence of SEQ ID NO: 69; and a LCDR3 comprising or consisting of the amino acid sequence of SEQ ID NO: 70.

In some embodiments, a heavy chain variable region of the anti-HER2 antibody or fragment thereof comprises or consists of the amino acid sequence of SEQ ID NO: 77, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the heavy chain variable region comprises: a HCDR1 comprising or consisting of the amino acid sequence of SEQ ID NO: 71; a HCDR2 comprising or consisting of the amino acid sequence of SEQ ID NO: 72; and a HCDR3 comprising or consisting of the amino acid sequence of SEQ ID NO: 73; and a light chain variable region of the anti-HER2 antibody or fragment thereof comprises or consists of the amino acid sequence of SEQ ID NO: 78 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the light chain variable region comprises: a LCDR1 comprising or consisting of the amino acid sequence of SEQ ID NO: 74; a LCDR2 comprising or consisting of the amino acid sequence of SEQ ID NO: 75; and a LCDR3 comprising or consisting of the amino acid sequence of SEQ ID NO: 76.

In some embodiments, a heavy chain variable region of the anti-HER2 antibody or fragment thereof comprises or consists of the amino acid sequence of SEQ ID NO: 77; and a light chain variable region of the anti-HER2 antibody or fragment thereof comprises or consists of the amino acid sequence of SEQ ID NO: 78. In some embodiments, a heavy chain variable region of the anti-HER2 antibody or fragment thereof consists of the amino acid sequence of SEQ ID NO: 77; and a light chain variable region of the anti-HER2 antibody or fragment thereof consists of the amino acid sequence of SEQ ID NO: 78.

In some embodiments, a heavy chain of the anti-HER2 antibody or fragment thereof comprises or consists of the amino acid sequence of SEQ ID NO: 79, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof. In some embodiments, a heavy chain of the anti-HER2 antibody or fragment thereof comprises or consists of the amino acid sequence of SEQ ID NO: 79.

In some embodiments, a light chain of the anti-HER2 antibody or fragment thereof comprises or consists of the amino acid sequence of SEQ ID NO: 80 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof. In some embodiments, a light chain of the anti-HER2 antibody or fragment thereof comprises or consists of the amino acid sequence of the amino acid sequence of SEQ ID NO: 80.

In some embodiments, a heavy chain of the anti-HER2 antibody or fragment thereof comprises or consists of the amino acid sequence of the amino acid sequence of SEQ ID NO: 79, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof; and a light chain of the anti-HER2 antibody or fragment thereof comprises or consists of the amino acid sequence of SEQ ID NO: 80 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof.

In some embodiments, a heavy chain of the anti-HER2 antibody or fragment thereof comprises or consists of the amino acid sequence of SEQ ID NO: 79, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the heavy chain variable region comprises: a HCDR1 comprising or consisting of the amino acid sequence of SEQ ID NO: 65; a HCDR2 comprising or consisting of the amino acid sequence of SEQ ID NO: 66; and a HCDR3 comprising or consisting of the amino acid sequence of SEQ ID NO: 67; and a light chain of the anti-HER2 antibody or fragment thereof comprises or consists of the amino acid sequence of SEQ ID NO: 80 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the light chain variable region comprises: a LCDR1 comprising or consisting of the amino acid sequence of SEQ ID NO: 68; a LCDR2 comprising or consisting of the amino acid sequence of SEQ ID NO: 69; and a LCDR3 comprising or consisting of the amino acid sequence of SEQ ID NO: 70.

In some embodiments, a heavy chain of the anti-HER2 antibody or fragment thereof comprises or consists of the amino acid sequence of SEQ ID NO: 79, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the heavy chain variable region comprises: a HCDR1 comprising or consisting of the amino acid sequence of SEQ ID NO: 71; a HCDR2 comprising or consisting of the amino acid sequence of SEQ ID NO: 72; and a HCDR3 comprising or consisting of the amino acid sequence of SEQ ID NO: 73; and a light chain of the anti-HER2 antibody or fragment thereof comprises or consists of the amino acid sequence of SEQ ID NO: 80 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the light chain variable region comprises: a LCDR1 comprising or consisting of the amino acid sequence of SEQ ID NO: 74; a LCDR2 comprising or consisting of the amino acid sequence of SEQ ID NO: 75; and a LCDR3 comprising or consisting of the amino acid sequence of SEQ ID NO: 76.

In some embodiments, a heavy chain of the anti-HER2 antibody or fragment thereof comprises or consists of the amino acid sequence of SEQ ID NO: 79; and a light chain of the anti-HER2 antibody or fragment thereof comprises or consists of the amino acid sequence of SEQ ID NO: 80. In some embodiments, a heavy chain of the anti-HER2 antibody or fragment thereof consists of the amino acid sequence of SEQ ID NO: 79; and a light chain of the anti-HER2 antibody or fragment thereof consists of the amino acid sequence of SEQ ID NO: 80.

In some embodiments, the anti-HER2 antibody or fragment thereof comprises or consists of two heavy chains comprising or consisting of the amino acid sequence of SEQ ID NO: 79; and two light chain comprising or consisting of the amino acid sequence of SEQ ID NO: 80. In some embodiments, the anti-HER2 antibody or fragment thereof comprises or consists of two heavy chains consisting of the amino acid sequence of SEQ ID NO: 79; and two light chains consisting of the amino acid sequence of SEQ ID NO: 80.

In some embodiments, the anti-HER2 antibody or fragment thereof is Pertuzumab or a fragment thereof. In some embodiments, the anti-HER2 antibody or fragment thereof is Pertuzumab.

### Anti-HER3 Antibodies and Fragments Thereof.

In some embodiments, the target expressed on the surface of a tumour cell is HER3. HER3 is a protein that in humans is encoded by the ERBB3 gene and which may also be known as human epidermal growth factor receptor 3 (HER-3) and Receptor tyrosine-protein kinase erbB-3.

In some embodiments, the antibody-drug conjugate comprises an anti-HER3 antibody or fragment thereof.

The anti-HER3 antibody or fragment thereof may specifically bind HER3. The anti-HER3 antibody or fragment thereof may block HER3 ligand-dependent and HER3 ligand-independent signalling. The anti-HER3 antibody or fragment thereof may inhibit HER3 ligand-dependent and Her3 ligand-independent AKT phosphorylation.

The anti-HER3 antibody may be a monoclonal antibody. In some embodiments, the anti-HER3 antibody or fragment thereof is a humanized antibody. In some embodiments, the anti-HER3 antibody or fragment thereof is a monoclonal humanized antibody. In some embodiments, the anti-HER3 antibody or fragment thereof is a human antibody. In some embodiments, the anti-HER3 antibody or fragment thereof is a monoclonal human antibody.

The fragment thereof may be any antigen-binding fragment thereof, for example a Fab, a Fab′, a F(ab′)2, a Fv, a scFv, a Fab′-SH, an sdAb, or a VHH. In some embodiments, the anti-HER3 antibody or fragment thereof is a full-length anti-HER3 antibody.

The anti-HER3 antibody or fragment thereof may comprise a variable region that specifically binds HER3. In some embodiments, the anti-HER3 antibody or fragment thereof comprises a heavy chain variable region and/or a light chain variable domain. In some embodiments, the anti-HER3 antibody or fragment thereof comprises a heavy chain variable region and a light chain variable domain. In some embodiments, the anti-HER3 antibody or fragment thereof comprises a constant region, preferably derived from a human antibody, preferably the constant region is selected from the constant region of human IgG1, IgG2, IgG3 or IgG4. In some embodiments, the anti-HER3 antibody or fragment thereof comprises a heavy chain and/or a light chain. In some embodiments, the anti-HER3 antibody or fragment thereof comprises a heavy chain and a light chain. In some embodiments, the anti-HER3 antibody or fragment thereof comprises or consists of two heavy chains and two light chains.

Example anti-HER3 antibody CDRs, variable region sequences, and heavy and light chain sequences are provided below.

In some embodiments, the anti-HER3 antibody or fragment thereof competes with any of the example anti-HER3 antibodies described herein for binding. In some embodiments, the anti-HER3 antibody or fragment thereof binds to domain 3 of human Her3 competitively with any of the example anti-HER3 antibodies described herein, and preferably, binds to amino acids from position 328 to position 499 of human HER3. Preferably, the anti-HER3 antibody or fragment thereof binds to the following spatial epitopes competitively with any of the example anti-HER3 antibodies described herein: Histidine at position 466, Tryptophan at position 470, Threonine at position 471, Threonine at position 478, (Aspartic acid-Isoleucine-lysine-Histidine-Asparagine) at positions 483 to 487, Isoleucine at position 484, Arginine at position 490 and Arginine at position 491 of human HER3.

### Example Anti-HER3 Antibody Sequences 1

| | |
|---|---|
| Kabat HCDR1 | TYGMH (SEQ ID NO: 190) |
| Kabat HCDR2 | VIWYDVSHKYYADSVKG (SEQ ID NO: 191) |
| Kabat HCDR3 | DWGDPDAFDI (SEQ ID NO: 192) |
| Kabat LCDR1 | RASQGISSYLA (SEQ ID NO: 193) |
| Kabat LCDR2 | AASTLOS (SEQ ID NO: 194) |
| Kabat LCDR3 | QQLNSYPLT (SEQ ID NO: 195) |
| IMGT HCDR1 | GFTFSTYG (SEQ ID NO: 196) |
| IMGT HCDR2 | IWYDVSHK (SEQ ID NO: 197) |
| IMGT HCDR3 | ARDWGDPDAFDI (SEQ ID NO: 198) |
| IMGT LCDR1 | QGISSY (SEQ ID NO: 199) |
| IMGT LCDR2 | AAS (SEQ ID NO: 200) |
| IMGT LCDR3 | QQLNSYPLT (SEQ ID NO: 201) |
| VH | EVQLVESGGGVVQPGRSLRLSCAASGFTFSTYGMHWVRQAPGKGLE |
| | WVAVIWYDVSHKYY ADSVKGRFTISRDNSKNTLSLQMNSLRAEDTA |
| | VYYCARDWGDPDAFDIWGQGTMVTVSS (SEQ ID NO: 202) |
| VL | DIQLTQSPSFLSASVGDRVTITCRASQGISSYLAWYQQKPGKAPKLLI |
| | YAASTLQSGVPSRFSGSGSGTEFTLTISSLQPEDFATYYCQQLNSYPLT |
| | FGGGTKVEIK (SEQ ID NO: 203) |
| Heavy chain | EVQLVESGGGVVQPGRSLRLSCAASGFTFSTYGMHWVRQAPGKGLE |
| | WVAVIWYDVSHKYYADSVKGRFTISRDNSKNTLSLQMNSLRAEDTA |
| | VYYCARDWGDPDAFDIWGQGTMVTVSSASTKGPSVFPLAPSSKSTS |
| | GGTAALGCLVKDYFPEPVTVSWNSGALTSGVHTFPAVLQSSGLYSLS |
| | SVVTVPSSSLGTQTYICNVNHKPSNTKVDKKVEPKSCDKTHTCPPCP |
| | APELLGGPSVFLFPPKPKDTLMISRTPEVTCVVVDVSHEDPEVKFNW |
| | YVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVK |
| | GFYPSDIAVEWESNGQPENNYKTTPPVLDSDGSFFLYSKLTVDKSRW |
| | QQGNVFSCSVMHEALHNHYTQKSLSLSPGK (SEQ ID NO: 204) |
| Light chain | DIQLTQSPSFLSASVGDRVTITCRASQGISSYLAWYQQKPGKAPKLLI |
| | YAASTLQSGVPSRFSGSGSGTEFTLTISSLQPEDFATYYCQQLNSYPLT |
| | FGGGTKVEIKRTVAAPSVFIFPPSDEQLKSGTASVVCLLNNFYPREAK |
| | VQWKVDNALQSGNSQESVTEQDSKDSTYSLSSTLTLSKADYEKHKV |
| | YACEVTHQGLSSPVTKSFNRGEC (SEQ ID NO: 205) |

In some embodiments, a heavy chain variable region of the anti-HER3 antibody or fragment thereof comprises: (i) a HCDR1 comprising or consisting of the amino acid sequence of SEQ ID NO: 190, or a variant thereof having up to three amino acid substitutions, additions or deletions; (ii) a HCDR2 comprising or consisting of the amino acid sequence of SEQ ID NO: 191, or a variant thereof having up to three amino acid substitutions, additions or deletions; and (iii) a HCDR3 comprising or consisting of the amino acid sequence of SEQ ID NO: 192, or a variant thereof having up to three amino acid substitutions, additions or deletions, wherein the CDRs are defined according to the Kabat numbering system.

In some embodiments, a light chain variable region of the anti-HER3 antibody or fragment thereof comprises: (i) a LCDR1 comprising or consisting of the amino acid sequence of SEQ ID NO: 193, or a variant thereof having up to three amino acid substitutions, additions or deletions; (ii) a LCDR2 comprising or consisting of the amino acid sequence of SEQ ID NO: 194, or a variant thereof having up to three amino acid substitutions, additions or deletions; and (iii) a LCDR3 comprising or consisting of the amino acid sequence of SEQ ID NO: 195, or a variant thereof having up to three amino acid substitutions, additions or deletions, wherein the CDRs are defined according to the Kabat numbering system.

In some embodiments, a heavy chain variable region of the anti-HER3 antibody or fragment thereof comprises: (i) a HCDR1 comprising or consisting of the amino acid sequence of SEQ ID NO: 190, or a variant thereof having up to three amino acid substitutions, additions or deletions; (ii) a HCDR2 comprising or consisting of the amino acid sequence of SEQ ID NO: 191, or a variant thereof having up to three amino acid substitutions, additions or deletions; and (iii) a HCDR3 comprising or consisting of the amino acid sequence of SEQ ID NO: 192, or a variant thereof having up to three amino acid substitutions, additions or deletions; and a light chain variable region of the anti-HER3 antibody or fragment thereof comprises: (i) a LCDR1 comprising or consisting of the amino acid sequence of SEQ ID NO: 193, or a variant thereof having up to three amino acid substitutions, additions or deletions; (ii) a LCDR2 comprising or consisting of the amino acid sequence of SEQ ID NO: 194, or a variant thereof having up to three amino acid substitutions, additions or deletions; and (iii) a LCDR3 comprising or consisting of the amino acid sequence of SEQ ID NO: 195, or a variant thereof having up to three amino acid substitutions, additions or deletions, wherein the CDRs are defined according to the Kabat numbering system.

In some embodiments, a heavy chain variable region of the anti-HER3 antibody or fragment thereof comprises: (i) a HCDR1 comprising or consisting of the amino acid sequence of SEQ ID NO: 196, or a variant thereof having up to three amino acid substitutions, additions or deletions; (ii) a HCDR2 comprising or consisting of the amino acid sequence of SEQ ID NO: 197, or a variant thereof having up to three amino acid substitutions, additions or deletions; and (iii) a HCDR3 comprising or consisting of the amino acid sequence of SEQ ID NO: 198, or a variant thereof having up to three amino acid substitutions, additions or deletions, wherein the CDRs are defined according to the IMGT numbering system.

In some embodiments, a light chain variable region of the anti-HER3 antibody or fragment thereof comprises: (i) a LCDR1 comprising or consisting of the amino acid sequence of SEQ ID NO: 199, or a variant thereof having up to three amino acid substitutions, additions or deletions; (ii) a LCDR2 comprising or consisting of the amino acid sequence of SEQ ID NO: 200; and (iii) a LCDR3 comprising or consisting of the amino acid sequence of SEQ ID NO: 201, or a variant thereof having up to three amino acid substitutions, additions or deletions, wherein the CDRs are defined according to the IMGT numbering system.

In some embodiments, a heavy chain variable region of the anti-HER3 antibody or fragment thereof comprises: (i) a HCDR1 comprising or consisting of the amino acid sequence of SEQ ID NO: 196, or a variant thereof having up to three amino acid substitutions, additions or deletions; (ii) a HCDR2 comprising or consisting of the amino acid sequence of SEQ ID NO: 197, or a variant thereof having up to three amino acid substitutions, additions or deletions; and (iii) a HCDR3 comprising or consisting of the amino acid sequence of SEQ ID NO: 198, or a variant thereof having up to three amino acid substitutions, additions or deletions; and a light chain variable region of the anti-HER3 antibody or fragment thereof comprises: (i) a LCDR1 comprising or consisting of the amino acid sequence of SEQ ID NO: 199, or a variant thereof having up to three amino acid substitutions, additions or deletions; (ii) a LCDR2 comprising or consisting of the amino acid sequence of SEQ ID NO: 200, or a variant thereof having up to three amino acid substitutions, additions or deletions; and (iii) a LCDR3 comprising or consisting of the amino acid sequence of SEQ ID NO: 201, or a variant thereof having up to three amino acid substitutions, additions or deletions, wherein the CDRs are defined according to the IMGT numbering system.

In some embodiments, the CDR variants have up to two amino acid substitutions, additions or deletions. In some embodiments, the CDR variants have up to one amino acid substitution, addition or deletion. In some embodiments, the CDR variants have up to three amino acid substitutions. In some embodiments, the CDR variants have up to two amino acid substitutions. In some embodiments, the CDR variants have up to one amino acid substitution.

In some embodiments, a heavy chain variable region of the anti-HER3 antibody or fragment thereof comprises or consists of the amino acid sequence of SEQ ID NO: 202, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof. In some embodiments, a heavy chain variable region of the anti-HER3 antibody or fragment thereof comprises or consists of the amino acid sequence of SEQ ID NO: 202.

In some embodiments, a light chain variable region of the anti-HER3 antibody or fragment thereof comprises or consists of the amino acid sequence of SEQ ID NO: 203 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof. In some embodiments, a light chain variable region of the anti-HER3 antibody or fragment thereof comprises or consists of the amino acid sequence of SEQ ID NO: 203.

In some embodiments, a heavy chain variable region of the anti-HER3 antibody or fragment thereof comprises or consists of the amino acid sequence of SEQ ID NO: 202, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, and a light chain variable region of the anti-HER3 antibody or fragment thereof comprises or consists of the amino acid sequence of SEQ ID NO: 203 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof.

In some embodiments, a heavy chain variable region of the anti-HER3 antibody or fragment thereof comprises or consists of the amino acid sequence of SEQ ID NO: 202, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the heavy chain variable region comprises: a HCDR1 comprising or consisting of the amino acid sequence of SEQ ID NO: 190; a HCDR2 comprising or consisting of the amino acid sequence of SEQ ID NO: 191; and a HCDR3 comprising or consisting of the amino acid sequence of SEQ ID NO: 192; and a light chain variable region of the anti-HER3 antibody or fragment thereof comprises or consists of the amino acid sequence of SEQ ID NO: 203 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the light chain variable region comprises: a LCDR1 comprising or consisting of the amino acid sequence of SEQ ID NO: 193; a LCDR2 comprising or consisting of the amino acid sequence of SEQ ID NO: 194; and a LCDR3 comprising or consisting of the amino acid sequence of SEQ ID NO: 195.

In some embodiments, a heavy chain variable region of the anti-HER3 antibody or fragment thereof comprises or consists of the amino acid sequence of SEQ ID NO: 202, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the heavy chain variable region comprises: a HCDR1 comprising or consisting of the amino acid sequence of SEQ ID NO: 196; a HCDR2 comprising or consisting of the amino acid sequence of SEQ ID NO: 197; and a HCDR3 comprising or consisting of the amino acid sequence of SEQ ID NO: 198; and a light chain variable region of the anti-HER3 antibody or fragment thereof comprises or consists of the amino acid sequence of SEQ ID NO: 203 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the light chain variable region comprises: a LCDR1 comprising or consisting of the amino acid sequence of SEQ ID NO: 199; a LCDR2 comprising or consisting of the amino acid sequence of SEQ ID NO: 200; and a LCDR3 comprising or consisting of the amino acid sequence of SEQ ID NO: 201.

In some embodiments, a heavy chain variable region of the anti-HER3 antibody or fragment thereof comprises or consists of the amino acid sequence of SEQ ID NO: 202; and a light chain variable region of the anti-HER3 antibody or fragment thereof comprises or consists of the amino acid sequence of SEQ ID NO: 203. In some embodiments, a heavy chain variable region of the anti-HER3 antibody or fragment thereof consists of the amino acid sequence of SEQ ID NO: 202; and a light chain variable region of the anti-HER3 antibody or fragment thereof consists of the amino acid sequence of SEQ ID NO: 203.

In some embodiments, a heavy chain of the anti-HER3 antibody or fragment thereof comprises or consists of the amino acid sequence of SEQ ID NO: 204, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof. In some embodiments, a heavy chain of the anti-HER3 antibody or fragment thereof comprises or consists of the amino acid sequence of SEQ ID NO: 204.

In some embodiments, a light chain of the anti-HER3 antibody or fragment thereof comprises or consists of the amino acid sequence of SEQ ID NO: 205 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof. In some embodiments, a light chain of the anti-HER3 antibody or fragment thereof comprises or consists of the amino acid sequence of the amino acid sequence of SEQ ID NO: 205.

In some embodiments, a heavy chain of the anti-HER3 antibody or fragment thereof comprises or consists of the amino acid sequence of the amino acid sequence of SEQ ID NO: 204, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof; and a light chain of the anti-HER3 antibody or fragment thereof comprises or consists of the amino acid sequence of SEQ ID NO: 205 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof.

In some embodiments, a heavy chain of the anti-HER3 antibody or fragment thereof comprises or consists of the amino acid sequence of SEQ ID NO: 204, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the heavy chain variable region comprises: a HCDR1 comprising or consisting of the amino acid sequence of SEQ ID NO: 190; a HCDR2 comprising or consisting of the amino acid sequence of SEQ ID NO: 191; and a HCDR3 comprising or consisting of the amino acid sequence of SEQ ID NO: 192; and a light chain of the anti-HER3 antibody or fragment thereof comprises or consists of the amino acid sequence of SEQ ID NO: 205 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the light chain variable region comprises: a LCDR1 comprising or consisting of the amino acid sequence of SEQ ID NO: 193; a LCDR2 comprising or consisting of the amino acid sequence of SEQ ID NO: 194; and a LCDR3 comprising or consisting of the amino acid sequence of SEQ ID NO: 195.

In some embodiments, a heavy chain of the anti-HER3 antibody or fragment thereof comprises or consists of the amino acid sequence of SEQ ID NO: 204, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the heavy chain variable region comprises: a HCDR1 comprising or consisting of the amino acid sequence of SEQ ID NO: 196; a HCDR2 comprising or consisting of the amino acid sequence of SEQ ID NO: 197; and a HCDR3 comprising or consisting of the amino acid sequence of SEQ ID NO: 198; and a light chain of the anti-HER3 antibody or fragment thereof comprises or consists of the amino acid sequence of SEQ ID NO: 205 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the light chain variable region comprises: a LCDR1 comprising or consisting of the amino acid sequence of SEQ ID NO: 199; a LCDR2 comprising or consisting of the amino acid sequence of SEQ ID NO: 200; and a LCDR3 comprising or consisting of the amino acid sequence of SEQ ID NO: 201.

In some embodiments, a heavy chain of the anti-HER3 antibody or fragment thereof comprises or consists of the amino acid sequence of SEQ ID NO: 204; and a light chain of the anti-HER3 antibody or fragment thereof comprises or consists of the amino acid sequence of SEQ ID NO: 205. In some embodiments, a heavy chain of the anti-HER3 antibody or fragment thereof consists of the amino acid sequence of SEQ ID NO: 204; and a light chain of the anti-HER3 antibody or fragment thereof consists of the amino acid sequence of SEQ ID NO: 205.

In some embodiments, the anti-HER3 antibody or fragment thereof comprises or consists of two heavy chains comprising or consisting of the amino acid sequence of SEQ ID NO: 204; and two light chain comprising or consisting of the amino acid sequence of SEQ ID NO: 205. In some embodiments, the anti-HER3 antibody or fragment thereof comprises or consists of two heavy chains consisting of the amino acid sequence of SEQ ID NO: 204; and two light chains consisting of the amino acid sequence of SEQ ID NO: 205.

In some embodiments, the anti-HER3 antibody or fragment thereof competes with an antibody comprising a heavy chain amino acid sequence as set forth in SEQ ID NO: 204 and a light chain amino acid sequence as set forth in SEQ ID NO: 205 for binding. In some embodiments, the anti-HER3 antibody or fragment thereof binds to domain 3 of human Her3 competitively with an antibody comprising a heavy chain amino acid sequence as set forth in SEQ ID NO: 204 and a light chain amino acid sequence as set forth in SEQ ID NO: 205, and preferably, binds to amino acids from position 328 to position 499 of human HER3. Preferably, the anti-HER3 antibody or fragment thereof binds to the following spatial epitopes competitively with an antibody comprising a heavy chain amino acid sequence as set forth in SEQ ID NO: 204 and a light chain amino acid sequence as set forth in SEQ ID NO: 205: Histidine at position 466, Tryptophan at position 470, Threonine at position 471, Threonine at position 478, (Aspartic acid-Isoleucine-lysine-Histidine-Asparagine) at positions 483 to 487, Isoleucine at position 484, Arginine at position 490 and Arginine at position 491 of human HER3.

| | |
|---|---|
| Kabat HCDR1 | GYFWS (SEQ ID NO: 206) |
| Kabat HCDR2 | YIHYSGRTNYNPSLKS (SEQ ID NO: 207) |
| Kabat HCDR3 | ETGGSGHWYFDL (SEQ ID NO: 208) |
| Kabat LCDR1 | RASQSISSYLI (SEQ ID NO: 209) |
| Kabat LCDR2 | AASSLOS (SEQ ID NO: 210) |
| Kabat LCDR3 | HQSYSTPWT (SEQ ID NO: 211) |
| IMGT HCDR1 | GGSISGYF (SEQ ID NO: 212) |
| IMGT HCDR2 | IHYSGRT (SEQ ID NO: 213) |
| IMGT HCDR3 | ARETGGSGHWYFDL (SEQ ID NO: 214) |
| IMGT LCDR1 | QSISSY (SEQ ID NO: 215) |
| IMGT LCDR2 | AAS (SEQ ID NO: 216) |
| IMGT LCDR3 | HQSYSTPWT (SEQ ID NO: 217) |
| VH | QVQLQESGPGLVKPSETLSLTCTVSGGSISGYFWSWIRQSPGKGLEWI |
| | GYIHYSGRTNYNPSLKSRVTISVDTSKNQFSLKLSSVTAADTAVYYC |
| | ARETGGSGHWYFDLWGRGNLVTVSS (SEQ ID NO: 218) |
| VL | DIQMTQSPSSLSASVGDRVTITCRASQSISSYLIWYQQKPGKAPKLLIY |
| | AASSLQSGVPSRFSGSGSGTDFTLTISSLQPEDFATYYCHQSYSTPWT |
| | FGQGTKVEIK (SEQ ID NO: 219) |

In some embodiments, a heavy chain variable region of the anti-HER3 antibody or fragment thereof comprises: (i) a HCDR1 comprising or consisting of the amino acid sequence of SEQ ID NO: 206, or a variant thereof having up to three amino acid substitutions, additions or deletions; (ii) a HCDR2 comprising or consisting of the amino acid sequence of SEQ ID NO: 207, or a variant thereof having up to three amino acid substitutions, additions or deletions; and (iii) a HCDR3 comprising or consisting of the amino acid sequence of SEQ ID NO: 208, or a variant thereof having up to three amino acid substitutions, additions or deletions, wherein the CDRs are defined according to the Kabat numbering system.

In some embodiments, a light chain variable region of the anti-HER3 antibody or fragment thereof comprises: (i) a LCDR1 comprising or consisting of the amino acid sequence of SEQ ID NO: 209, or a variant thereof having up to three amino acid substitutions, additions or deletions; (ii) a LCDR2 comprising or consisting of the amino acid sequence of SEQ ID NO: 210, or a variant thereof having up to three amino acid substitutions, additions or deletions; and (iii) a LCDR3 comprising or consisting of the amino acid sequence of SEQ ID NO: 211, or a variant thereof having up to three amino acid substitutions, additions or deletions, wherein the CDRs are defined according to the Kabat numbering system.

In some embodiments, a heavy chain variable region of the anti-HER3 antibody or fragment thereof comprises: (i) a HCDR1 comprising or consisting of the amino acid sequence of SEQ ID NO: 206, or a variant thereof having up to three amino acid substitutions, additions or deletions; (ii) a HCDR2 comprising or consisting of the amino acid sequence of SEQ ID NO: 207, or a variant thereof having up to three amino acid substitutions, additions or deletions; and (iii) a HCDR3 comprising or consisting of the amino acid sequence of SEQ ID NO: 208, or a variant thereof having up to three amino acid substitutions, additions or deletions; and a light chain variable region of the anti-HER3 antibody or fragment thereof comprises: (i) a LCDR1 comprising or consisting of the amino acid sequence of SEQ ID NO: 209, or a variant thereof having up to three amino acid substitutions, additions or deletions; (ii) a LCDR2 comprising or consisting of the amino acid sequence of SEQ ID NO: 210, or a variant thereof having up to three amino acid substitutions, additions or deletions; and (iii) a LCDR3 comprising or consisting of the amino acid sequence of SEQ ID NO: 211, or a variant thereof having up to three amino acid substitutions, additions or deletions, wherein the CDRs are defined according to the Kabat numbering system.

In some embodiments, a heavy chain variable region of the anti-HER3 antibody or fragment thereof comprises: (i) a HCDR1 comprising or consisting of the amino acid sequence of SEQ ID NO: 212, or a variant thereof having up to three amino acid substitutions, additions or deletions; (ii) a HCDR2 comprising or consisting of the amino acid sequence of SEQ ID NO: 213, or a variant thereof having up to three amino acid substitutions, additions or deletions; and (iii) a HCDR3 comprising or consisting of the amino acid sequence of SEQ ID NO: 214, or a variant thereof having up to three amino acid substitutions, additions or deletions, wherein the CDRs are defined according to the IMGT numbering system.

In some embodiments, a light chain variable region of the anti-HER3 antibody or fragment thereof comprises: (i) a LCDR1 comprising or consisting of the amino acid sequence of SEQ ID NO: 215, or a variant thereof having up to three amino acid substitutions, additions or deletions; (ii) a LCDR2 comprising or consisting of the amino acid sequence of SEQ ID NO: 216; and (iii) a LCDR3 comprising or consisting of the amino acid sequence of SEQ ID NO: 217, or a variant thereof having up to three amino acid substitutions, additions or deletions, wherein the CDRs are defined according to the IMGT numbering system.

In some embodiments, a heavy chain variable region of the anti-HER3 antibody or fragment thereof comprises: (i) a HCDR1 comprising or consisting of the amino acid sequence of SEQ ID NO: 212, or a variant thereof having up to three amino acid substitutions, additions or deletions; (ii) a HCDR2 comprising or consisting of the amino acid sequence of SEQ ID NO: 213, or a variant thereof having up to three amino acid substitutions, additions or deletions; and (iii) a HCDR3 comprising or consisting of the amino acid sequence of SEQ ID NO: 214, or a variant thereof having up to three amino acid substitutions, additions or deletions; and a light chain variable region of the anti-HER3 antibody or fragment thereof comprises: (i) a LCDR1 comprising or consisting of the amino acid sequence of SEQ ID NO: 215, or a variant thereof having up to three amino acid substitutions, additions or deletions; (ii) a LCDR2 comprising or consisting of the amino acid sequence of SEQ ID NO: 216, or a variant thereof having up to three amino acid substitutions, additions or deletions; and (iii) a LCDR3 comprising or consisting of the amino acid sequence of SEQ ID NO: 217, or a variant thereof having up to three amino acid substitutions, additions or deletions, wherein the CDRs are defined according to the IMGT numbering system.

In some embodiments, the CDR variants have up to two amino acid substitutions, additions or deletions. In some embodiments, the CDR variants have up to one amino acid substitution, addition or deletion. In some embodiments, the CDR variants have up to three amino acid substitutions. In some embodiments, the CDR variants have up to two amino acid substitutions. In some embodiments, the CDR variants have up to one amino acid substitution.

In some embodiments, a heavy chain variable region of the anti-HER3 antibody or fragment thereof comprises or consists of the amino acid sequence of SEQ ID NO: 218, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof. In some embodiments, a heavy chain variable region of the anti-HER3 antibody or fragment thereof comprises or consists of the amino acid sequence of SEQ ID NO: 218.

In some embodiments, a light chain variable region of the anti-HER3 antibody or fragment thereof comprises or consists of the amino acid sequence of SEQ ID NO: 219 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof. In some embodiments, a light chain variable region of the anti-HER3 antibody or fragment thereof comprises or consists of the amino acid sequence of SEQ ID NO: 219.

In some embodiments, a heavy chain variable region of the anti-HER3 antibody or fragment thereof comprises or consists of the amino acid sequence of SEQ ID NO: 218, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, and a light chain variable region of the anti-HER3 antibody or fragment thereof comprises or consists of the amino acid sequence of SEQ ID NO: 219 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof.

In some embodiments, a heavy chain variable region of the anti-HER3 antibody or fragment thereof comprises or consists of the amino acid sequence of SEQ ID NO: 218, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the heavy chain variable region comprises: a HCDR1 comprising or consisting of the amino acid sequence of SEQ ID NO: 206; a HCDR2 comprising or consisting of the amino acid sequence of SEQ ID NO: 207; and a HCDR3 comprising or consisting of the amino acid sequence of SEQ ID NO: 208; and a light chain variable region of the anti-HER3 antibody or fragment thereof comprises or consists of the amino acid sequence of SEQ ID NO: 219 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the light chain variable region comprises: a LCDR1 comprising or consisting of the amino acid sequence of SEQ ID NO: 209; a LCDR2 comprising or consisting of the amino acid sequence of SEQ ID NO: 210; and a LCDR3 comprising or consisting of the amino acid sequence of SEQ ID NO: 211.

In some embodiments, a heavy chain variable region of the anti-HER3 antibody or fragment thereof comprises or consists of the amino acid sequence of SEQ ID NO: 218, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the heavy chain variable region comprises: a HCDR1 comprising or consisting of the amino acid sequence of SEQ ID NO: 212; a HCDR2 comprising or consisting of the amino acid sequence of SEQ ID NO: 213; and a HCDR3 comprising or consisting of the amino acid sequence of SEQ ID NO: 214; and a light chain variable region of the anti-HER3 antibody or fragment thereof comprises or consists of the amino acid sequence of SEQ ID NO: 219 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the light chain variable region comprises: a LCDR1 comprising or consisting of the amino acid sequence of SEQ ID NO: 215; a LCDR2 comprising or consisting of the amino acid sequence of SEQ ID NO: 216; and a LCDR3 comprising or consisting of the amino acid sequence of SEQ ID NO: 217.

In some embodiments, a heavy chain variable region of the anti-HER3 antibody or fragment thereof comprises or consists of the amino acid sequence of SEQ ID NO: 218; and a light chain variable region of the anti-HER3 antibody or fragment thereof comprises or consists of the amino acid sequence of SEQ ID NO: 219. In some embodiments, a heavy chain variable region of the anti-HER3 antibody or fragment thereof consists of the amino acid sequence of SEQ ID NO: 218; and a light chain variable region of the anti-HER3 antibody or fragment thereof consists of the amino acid sequence of SEQ ID NO: 219.

| | |
|---|---|
| Kabat HCDR1 | GYFWS (SEQ ID NO: 220) |
| Kabat HCDR2 | YIHYSGRTNYNPSLKS (SEQ ID NO: 221) |
| Kabat HCDR3 | ETGGSGHWYFDL (SEQ ID NO: 222) |
| Kabat LCDR1 | RASQSISSYLI (SEQ ID NO: 223) |
| Kabat LCDR2 | AASSLOS (SEQ ID NO: 224) |
| Kabat LCDR3 | HQSYSTPWT (SEQ ID NO: 225) |
| IMGT HCDR1 | GGSISGYF (SEQ ID NO: 226) |
| IMGT HCDR2 | IHYSGRT (SEQ ID NO: 227) |
| IMGT HCDR3 | ARETGGSGHWYFDL (SEQ ID NO: 228) |
| IMGT LCDR1 | QSISSY (SEQ ID NO: 229) |
| IMGT LCDR2 | AAS (SEQ ID NO: 230) |
| IMGT LCDR3 | HQSYSTPWT (SEQ ID NO: 231) |
| VH | QVQLQESGPGLVKPSETLSLTCTVSGGSISGYFWSWIRQSPGQGLEWI |
| | GYIHYSGRTNYNPSLKSRVTISVDTSKNQFSLQLSSVTAADTAVYYC |
| | ARETGGSGHWYFDLWGRGNLVTVSS (SEQ ID NO: 232) |
| VL | DIQMTQSPSSLSASVGDRVTITCRASQSISSYLIWYQQKPGQAPRLLIY |
| | AASSLQSGVPSRFSGSGSGTDFTLTISSLQPEDFATYYCHQSYSTPWT |
| | FGQGTKVEIK (SEQ ID NO: 233) |

In some embodiments, a heavy chain variable region of the anti-HER3 antibody or fragment thereof comprises: (i) a HCDR1 comprising or consisting of the amino acid sequence of SEQ ID NO: 220, or a variant thereof having up to three amino acid substitutions, additions or deletions; (ii) a HCDR2 comprising or consisting of the amino acid sequence of SEQ ID NO: 221, or a variant thereof having up to three amino acid substitutions, additions or deletions; and (iii) a HCDR3 comprising or consisting of the amino acid sequence of SEQ ID NO: 222, or a variant thereof having up to three amino acid substitutions, additions or deletions, wherein the CDRs are defined according to the Kabat numbering system.

In some embodiments, a light chain variable region of the anti-HER3 antibody or fragment thereof comprises: (i) a LCDR1 comprising or consisting of the amino acid sequence of SEQ ID NO: 223, or a variant thereof having up to three amino acid substitutions, additions or deletions; (ii) a LCDR2 comprising or consisting of the amino acid sequence of SEQ ID NO: 224, or a variant thereof having up to three amino acid substitutions, additions or deletions; and (iii) a LCDR3 comprising or consisting of the amino acid sequence of SEQ ID NO: 225, or a variant thereof having up to three amino acid substitutions, additions or deletions, wherein the CDRs are defined according to the Kabat numbering system.

In some embodiments, a heavy chain variable region of the anti-HER3 antibody or fragment thereof comprises: (i) a HCDR1 comprising or consisting of the amino acid sequence of SEQ ID NO: 220, or a variant thereof having up to three amino acid substitutions, additions or deletions; (ii) a HCDR2 comprising or consisting of the amino acid sequence of SEQ ID NO: 221, or a variant thereof having up to three amino acid substitutions, additions or deletions; and (iii) a HCDR3 comprising or consisting of the amino acid sequence of SEQ ID NO: 222, or a variant thereof having up to three amino acid substitutions, additions or deletions; and a light chain variable region of the anti-HER3 antibody or fragment thereof comprises: (i) a LCDR1 comprising or consisting of the amino acid sequence of SEQ ID NO: 223, or a variant thereof having up to three amino acid substitutions, additions or deletions; (ii) a LCDR2 comprising or consisting of the amino acid sequence of SEQ ID NO: 224, or a variant thereof having up to three amino acid substitutions, additions or deletions; and (iii) a LCDR3 comprising or consisting of the amino acid sequence of SEQ ID NO: 225, or a variant thereof having up to three amino acid substitutions, additions or deletions, wherein the CDRs are defined according to the Kabat numbering system.

In some embodiments, a heavy chain variable region of the anti-HER3 antibody or fragment thereof comprises: (i) a HCDR1 comprising or consisting of the amino acid sequence of SEQ ID NO: 226, or a variant thereof having up to three amino acid substitutions, additions or deletions; (ii) a HCDR2 comprising or consisting of the amino acid sequence of SEQ ID NO: 227, or a variant thereof having up to three amino acid substitutions, additions or deletions; and (iii) a HCDR3 comprising or consisting of the amino acid sequence of SEQ ID NO: 228, or a variant thereof having up to three amino acid substitutions, additions or deletions, wherein the CDRs are defined according to the IMGT numbering system.

In some embodiments, a light chain variable region of the anti-HER3 antibody or fragment thereof comprises: (i) a LCDR1 comprising or consisting of the amino acid sequence of SEQ ID NO: 229, or a variant thereof having up to three amino acid substitutions, additions or deletions; (ii) a LCDR2 comprising or consisting of the amino acid sequence of SEQ ID NO: 230; and (iii) a LCDR3 comprising or consisting of the amino acid sequence of SEQ ID NO: 231, or a variant thereof having up to three amino acid substitutions, additions or deletions, wherein the CDRs are defined according to the IMGT numbering system.

In some embodiments, a heavy chain variable region of the anti-HER3 antibody or fragment thereof comprises: (i) a HCDR1 comprising or consisting of the amino acid sequence of SEQ ID NO: 226, or a variant thereof having up to three amino acid substitutions, additions or deletions; (ii) a HCDR2 comprising or consisting of the amino acid sequence of SEQ ID NO: 227, or a variant thereof having up to three amino acid substitutions, additions or deletions; and (iii) a HCDR3 comprising or consisting of the amino acid sequence of SEQ ID NO: 228, or a variant thereof having up to three amino acid substitutions, additions or deletions; and a light chain variable region of the anti-HER3 antibody or fragment thereof comprises: (i) a LCDR1 comprising or consisting of the amino acid sequence of SEQ ID NO: 229, or a variant thereof having up to three amino acid substitutions, additions or deletions; (ii) a LCDR2 comprising or consisting of the amino acid sequence of SEQ ID NO: 230, or a variant thereof having up to three amino acid substitutions, additions or deletions; and (iii) a LCDR3 comprising or consisting of the amino acid sequence of SEQ ID NO: 231, or a variant thereof having up to three amino acid substitutions, additions or deletions, wherein the CDRs are defined according to the IMGT numbering system.

In some embodiments, the CDR variants have up to two amino acid substitutions, additions or deletions. In some embodiments, the CDR variants have up to one amino acid substitution, addition or deletion. In some embodiments, the CDR variants have up to three amino acid substitutions. In some embodiments, the CDR variants have up to two amino acid substitutions. In some embodiments, the CDR variants have up to one amino acid substitution.

In some embodiments, a heavy chain variable region of the anti-HER3 antibody or fragment thereof comprises or consists of the amino acid sequence of SEQ ID NO: 232, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof. In some embodiments, a heavy chain variable region of the anti-HER3 antibody or fragment thereof comprises or consists of the amino acid sequence of SEQ ID NO: 232.

In some embodiments, a light chain variable region of the anti-HER3 antibody or fragment thereof comprises or consists of the amino acid sequence of SEQ ID NO: 233 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof. In some embodiments, a light chain variable region of the anti-HER3 antibody or fragment thereof comprises or consists of the amino acid sequence of SEQ ID NO: 233.

In some embodiments, a heavy chain variable region of the anti-HER3 antibody or fragment thereof comprises or consists of the amino acid sequence of SEQ ID NO: 232, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, and a light chain variable region of the anti-HER3 antibody or fragment thereof comprises or consists of the amino acid sequence of SEQ ID NO: 233 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof.

In some embodiments, a heavy chain variable region of the anti-HER3 antibody or fragment thereof comprises or consists of the amino acid sequence of SEQ ID NO: 232, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the heavy chain variable region comprises: a HCDR1 comprising or consisting of the amino acid sequence of SEQ ID NO: 220; a HCDR2 comprising or consisting of the amino acid sequence of SEQ ID NO: 221; and a HCDR3 comprising or consisting of the amino acid sequence of SEQ ID NO: 222; and a light chain variable region of the anti-HER3 antibody or fragment thereof comprises or consists of the amino acid sequence of SEQ ID NO: 233 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the light chain variable region comprises: a LCDR1 comprising or consisting of the amino acid sequence of SEQ ID NO: 223; a LCDR2 comprising or consisting of the amino acid sequence of SEQ ID NO: 224; and a LCDR3 comprising or consisting of the amino acid sequence of SEQ ID NO: 225.

In some embodiments, a heavy chain variable region of the anti-HER3 antibody or fragment thereof comprises or consists of the amino acid sequence of SEQ ID NO: 232, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the heavy chain variable region comprises: a HCDR1 comprising or consisting of the amino acid sequence of SEQ ID NO: 226; a HCDR2 comprising or consisting of the amino acid sequence of SEQ ID NO: 227; and a HCDR3 comprising or consisting of the amino acid sequence of SEQ ID NO: 228; and a light chain variable region of the anti-HER3 antibody or fragment thereof comprises or consists of the amino acid sequence of SEQ ID NO: 233 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the light chain variable region comprises: a LCDR1 comprising or consisting of the amino acid sequence of SEQ ID NO: 229; a LCDR2 comprising or consisting of the amino acid sequence of SEQ ID NO: 230; and a LCDR3 comprising or consisting of the amino acid sequence of SEQ ID NO: 231.

In some embodiments, a heavy chain variable region of the anti-HER3 antibody or fragment thereof comprises or consists of the amino acid sequence of SEQ ID NO: 232; and a light chain variable region of the anti-HER3 antibody or fragment thereof comprises or consists of the amino acid sequence of SEQ ID NO: 233. In some embodiments, a heavy chain variable region of the anti-HER3 antibody or fragment thereof consists of the amino acid sequence of SEQ ID NO: 232; and a light chain variable region of the anti-HER3 antibody or fragment thereof consists of the amino acid sequence of SEQ ID NO: 233.

### Further Antibodies and Fragments Thereof.

In some embodiments, the target expressed on the surface of a tumour cell is selected from CD30, Nectin-4, TF, B7-H4, ITGB6, and HER2.

In some embodiments, the antibody-drug conjugate comprises an anti-CD30 antibody or fragment thereof, an anti-Nectin-4 antibody or fragment thereof, an anti-TF antibody or fragment thereof, an anti-B7-H4 antibody or fragment thereof, an anti-ITGB6 antibody or fragment thereof, or an anti-HER2 antibody or fragment thereof.

In some embodiments, the antibody-drug conjugate comprises an anti-CD30 antibody, an anti-Nectin-4 antibody, an anti-TF antibody, an anti-B7-H4 antibody, an anti-ITGB6 antibody, or an anti-HER2 antibody.

In some embodiments, the antibody-drug conjugate comprises Brentuximab or a fragment thereof, Enfortumab or a fragment thereof, Tisotumab or a fragment thereof, Felmetatug or a fragment thereof, Sigvotatug or a fragment thereof, or Disitamab or a fragment thereof.

In some embodiments, the antibody-drug conjugate comprises Brentuximab, Enfortumab, Tisotumab, Felmetatug, Sigvotatug, or Disitamab.

Example CDRs, variable region sequences, and heavy and light chain sequences are provided below.

### Anti-CD30 Antibodies and Fragments Thereof.

In some embodiments, the target expressed on the surface of a tumour cell is CD30. CD30 is a protein that in humans is encoded by the TNFRSF8 gene and which may also be known as TNFRSF8 (TNF receptor superfamily member 8).

In some embodiments, the antibody-drug conjugate comprises an anti-CD30 antibody or fragment thereof.

The anti-CD30 antibody or fragment thereof may specifically bind CD30. The anti-CD30 antibody may be a monoclonal antibody. In some embodiments, the anti-CD30 antibody or fragment thereof is a humanized antibody. In some embodiments, the anti-CD30 antibody or fragment thereof is a monoclonal humanized antibody. The fragment thereof may be any antigen-binding fragment thereof, for example a Fab, a Fab′, a F(ab′)2, a Fv, an ScFv, a Fab′-SH, an sdAb, or a VHH. In some embodiments, the anti-CD30 antibody or fragment thereof is a full-length anti-CD30 antibody.

The anti-CD30 antibody or fragment thereof may comprise a variable region that specifically binds CD30. In some embodiments, the anti-CD30 antibody or fragment thereof comprises a heavy chain variable region and/or a light chain variable domain. In some embodiments, the anti-CD30 antibody or fragment thereof comprises a heavy chain variable region and a light chain variable domain. In some embodiments, the anti-CD30 antibody or fragment thereof comprises a constant region, preferably derived from a human antibody, preferably the constant region is selected from the constant region of human IgG1, IgG2, IgG3 or IgG4 (e.g. IgG1). In some embodiments, the anti-CD30 antibody or fragment thereof comprises a heavy chain and/or a light chain. In some embodiments, the anti-CD30 antibody or fragment thereof comprises a heavy chain and a light chain. In some embodiments, the anti-CD30 antibody or fragment thereof comprises or consists of two heavy chains and two light chains.

Example anti-CD30 antibody CDRs, variable region sequences, and heavy and light chain sequences are provided below.

| | |
|---|---|
| Kabat HCDR1 | DYYIT (SEQ ID NO: 234) |
| Kabat HCDR2 | WIYPGSGNTKYNEKFKG (SEQ ID NO: 235) |
| Kabat HCDR3 | YGNYWFAY (SEQ ID NO: 236) |
| Kabat LCDR1 | KASQSVDFDGDSYMN (SEQ ID NO: 237) |
| Kabat LCDR2 | AASNLES (SEQ ID NO: 238) |
| Kabat LCDR3 | QQSNEDPWT (SEQ ID NO: 239) |
| IMGT HCDR1 | GYTFTDYY (SEQ ID NO: 240) |
| IMGT HCDR2 | IYPGSGNT (SEQ ID NO: 241) |
| IMGT HCDR3 | ANYGNYWFAY (SEQ ID NO: 242) |
| IMGT LCDR1 | QSVDFDGDSY (SEQ ID NO: 243) |
| IMGT LCDR2 | AAS (SEQ ID NO: 244) |
| IMGT LCDR3 | QQSNEDPWT (SEQ ID NO: 245) |
| VH | QIQLQQSGPEVVKPGASVKISCKASGYTFTDYYITWVKQKPGQGLE |
| | WIGWIYPGSGNTKYNEKFKGKATLTVDTSSSTAFMQLSSLTSEDTAV |
| | YFCANYGNYWFAYWGQGTQVTVSA (SEQ ID NO: 246) |
| VL | DIVLTQSPASLAVSLGQRATISCKASQSVDFDGDSYMNWYQQKPGQ |
| | PPKVLIYAASNLESGIPARFSGSGSGTDFTLNIHPVEEEDAATYYCQQ |
| | SNEDPWTFGGGTKLEIK (SEQ ID NO: 247) |
| Heavy chain | QIQLQQSGPEVVKPGASVKISCKASGYTFTDYYITWVKQKPGQGLE |
| | WIGWIYPGSGNTKYNEKFKGKATLTVDTSSSTAFMQLSSLTSEDTAV |
| | YFCANYGNYWFAYWGQGTQVTVSAASTKGPSVFPLAPSSKSTSGGT |
| | AALGCLVKDYFPEPVTVSWNSGALTSGVHTFPAVLQSSGLYSLSSVV |
| | TVPSSSLGTQTYICNVNHKPSNTKVDKKVEPKSCDKTHTCPPCPAPEL |
| | LGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNK |
| | ALPAPIEKTISKAKGQPREPQVYTLPPSRDELTKNQVSLTCLVKGFYP |
| | SDIAVEWESNGQPENNYKTTPPVLDSDGSFFLYSKLTVDKSRWQQG |
| | NVFSCSVMHEALHNHYTQKSLSLSPG (SEQ ID NO: 248) |
| Light chain | DIVLTQSPASLAVSLGQRATISCKASQSVDFDGDSYMNWYQQKPGQ |
| | PPKVLIYAASNLESGIPARFSGSGSGTDFTLNIHPVEEEDAATYYCQQ |
| | SNEDPWTFGGGTKLEIKRTVAAPSVFIFPPSDEQLKSGTASVVCLLNN |
| | FYPREAKVQWKVDNALQSGNSQESVTEQDSKDSTYSLSSTLTLSKA |
| | DYEKHKVYACEVTHQGLSSPVTKSFNRGEC (SEQ ID NO: 249) |

In some embodiments, a heavy chain variable region of the anti-CD30 antibody or fragment thereof comprises: (i) a HCDR1 comprising or consisting of the amino acid sequence of SEQ ID NO: 234; (ii) a HCDR2 comprising or consisting of the amino acid sequence of SEQ ID NO: 235, and (iii) a HCDR3 comprising or consisting of the amino acid sequence of SEQ ID NO: 236; and a light chain variable region of the anti-CD30 antibody or fragment thereof comprises: (i) a LCDR1 comprising or consisting of the amino acid sequence of SEQ ID NO: 237; (ii) a LCDR2 comprising or consisting of the amino acid sequence of SEQ ID NO: 238; and (iii) a LCDR3 comprising or consisting of the amino acid sequence of SEQ ID NO: 239, wherein the CDRs are defined according to the Kabat numbering system.

In some embodiments, a heavy chain variable region of the anti-CD30 antibody or fragment thereof comprises: (i) a HCDR1 comprising or consisting of the amino acid sequence of SEQ ID NO: 240; (ii) a HCDR2 comprising or consisting of the amino acid sequence of SEQ ID NO: 241; and (iii) a HCDR3 comprising or consisting of the amino acid sequence of SEQ ID NO: 242; and a light chain variable region of the anti-CD30 antibody or fragment thereof comprises: (i) a LCDR1 comprising or consisting of the amino acid sequence of SEQ ID NO: 243; (ii) a LCDR2 comprising or consisting of the amino acid sequence of SEQ ID NO: 244; and (iii) a LCDR3 comprising or consisting of the amino acid sequence of SEQ ID NO: 245, wherein the CDRs are defined according to the IMGT numbering system.

In some embodiments, a heavy chain variable region of the anti-CD30 antibody or fragment thereof comprises or consists of the amino acid sequence of SEQ ID NO: 246, or an amino acid sequence having at least 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof; and a light chain variable region of the anti-CD30 antibody or fragment thereof comprises or consists of the amino acid sequence of SEQ ID NO: 247 or an amino acid sequence having at least 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof.

In some embodiments, a heavy chain variable region of the anti-CD30 antibody or fragment thereof comprises or consists of the amino acid sequence of SEQ ID NO: 246; and a light chain variable region of the anti-CD30 antibody or fragment thereof comprises or consists of the amino acid sequence of SEQ ID NO: 247. In some embodiments, a heavy chain variable region of the anti-CD30 antibody or fragment thereof consists of the amino acid sequence of SEQ ID NO: 246; and a light chain variable region of the anti-CD30 antibody or fragment thereof consists of the amino acid sequence of SEQ ID NO: 247.

In some embodiments, a heavy chain of the anti-CD30 antibody or fragment thereof comprises or consists of the amino acid sequence of the amino acid sequence of SEQ ID NO: 248, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof; and a light chain of the anti-CD30 antibody or fragment thereof comprises or consists of the amino acid sequence of SEQ ID NO: 249 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof.

In some embodiments, a heavy chain of the anti-CD30 antibody or fragment thereof comprises or consists of the amino acid sequence of SEQ ID NO: 248; and a light chain of the anti-CD30 antibody or fragment thereof comprises or consists of the amino acid sequence of SEQ ID NO: 249. In some embodiments, a heavy chain of the anti-CD30 antibody or fragment thereof consists of the amino acid sequence of SEQ ID NO: 248; and a light chain of the anti-CD30 antibody or fragment thereof consists of the amino acid sequence of SEQ ID NO: 249.

In some embodiments, the anti-CD30 antibody or fragment thereof comprises or consists of two heavy chains comprising or consisting of the amino acid sequence of SEQ ID NO: 248; and two light chain comprising or consisting of the amino acid sequence of SEQ ID NO: 249.

In some embodiments, the anti-CD30 antibody or fragment thereof comprises or consists of two heavy chains consisting of the amino acid sequence of SEQ ID NO: 248; and two light chains consisting of the amino acid sequence of SEQ ID NO: 249.

In some embodiments, the anti-CD30 antibody or fragment thereof is Brentuximab or a fragment thereof. In some embodiments, the anti-CD30 antibody or fragment thereof is Brentuximab.

### Anti-Nectin-4 Antibodies and Fragments Thereof.

In some embodiments, the target expressed on the surface of a tumour cell is Nectin-4.

Nectin-4 is a protein that in humans is encoded by the NECTIN4 gene and which may also be known as Nectin Cell Adhesion Molecule 4.

In some embodiments, the antibody-drug conjugate comprises an anti-Nectin-4 antibody or fragment thereof.

The anti-Nectin-4 antibody or fragment thereof may specifically bind Nectin-4. The anti-Nectin-4 antibody may be a monoclonal antibody. In some embodiments, the anti-Nectin-4 antibody or fragment thereof is a human antibody. In some embodiments, the anti-Nectin-4 antibody or fragment thereof is a monoclonal human antibody. The fragment thereof may be any antigen-binding fragment thereof, for example a Fab, a Fab′, a F(ab′)2, a Fv, an ScFv, a Fab′-SH, an sdAb, or a VHH. In some embodiments, the anti-Nectin-4 antibody or fragment thereof is a full-length anti-Nectin-4 antibody.

The anti-Nectin-4 antibody or fragment thereof may comprise a variable region that specifically binds Nectin-4. In some embodiments, the anti-Nectin-4 antibody or fragment thereof comprises a heavy chain variable region and/or a light chain variable domain. In some embodiments, the anti-Nectin-4 antibody or fragment thereof comprises a heavy chain variable region and a light chain variable domain. In some embodiments, the anti-Nectin-4 antibody or fragment thereof comprises a constant region, preferably derived from a human antibody, preferably the constant region is selected from the constant region of human IgG1, IgG2, IgG3 or IgG4 (e.g. IgG1). In some embodiments, the anti-Nectin-4 antibody or fragment thereof comprises a heavy chain and/or a light chain. In some embodiments, the anti-Nectin-4 antibody or fragment thereof comprises a heavy chain and a light chain. In some embodiments, the anti-Nectin-4 antibody or fragment thereof comprises or consists of two heavy chains and two light chains.

Example anti-Nectin-4 antibody CDRs, variable region sequences, and heavy and light chain sequences are provided below.

| | |
|---|---|
| Kabat HCDR1 | SYNMN (SEQ ID NO: 250) |
| Kabat HCDR2 | YISSSSSTIYYADSVKG (SEQ ID NO: 251) |
| Kabat HCDR3 | AYYYGMDV (SEQ ID NO: 252) |
| Kabat LCDR1 | RASQGISGWLA (SEQ ID NO: 253) |
| Kabat LCDR2 | AASTLOS (SEQ ID NO: 254) |
| Kabat LCDR3 | QQANSFPPT (SEQ ID NO: 255) |
| IMGT HCDR1 | GFTFSSYN (SEQ ID NO: 256) |
| IMGT HCDR2 | ISSSSSTI (SEQ ID NO: 257) |
| IMGT HCDR3 | ARAYYYGMDV (SEQ ID NO: 258) |
| IMGT LCDR1 | QGISGW (SEQ ID NO: 259) |
| IMGT LCDR2 | AAS (SEQ ID NO: 260) |
| IMGT LCDR3 | QQANSFPPT (SEQ ID NO: 261) |
| VH | EVQLVESGGGLVQPGGSLRLSCAASGFTFSSYNMNWVRQAPGKGLE |
| | WVSYISSSSSTIYYADSVKGRFTISRDNAKNSLSLQMNSLRDEDTAVY |
| | YCARAYYYGMDVWGQGTTVTVSS (SEQ ID NO: 262) |
| VL | DIQMTQSPSSVSASVGDRVTITCRASQGISGWLAWYQQKPGKAPKFL |
| | IYAASTLQSGVPSRFSGSGSGTDFTLTISSLQPEDFATYYCQQANSFPP |
| | TFGGGTKVEIK (SEQ ID NO: 263) |
| Heavy chain | EVQLVESGGGLVQPGGSLRLSCAASGFTFSSYNMNWVRQAPGKGLE |
| | WVSYISSSSSTIYYADSVKGRFTISRDNAKNSLSLQMNSLRDEDTAVY |
| | YCARAYYYGMDVWGQGTTVTVSSASTKGPSVFPLAPSSKSTSGGTA |
| | ALGCLVKDYFPEPVTVSWNSGALTSGVHTFPAVLQSSGLYSLSSVVT |
| | VPSSSLGTQTYICNVNHKPSNTKVDKRVEPKSCDKTHTCPPCPAPELL |
| | GGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKA |
| | LPAPIEKTISKAKGQPREPQVYTLPPSREEMTKNQVSLTCLVKGFYPS |
| | DIAVEWESNGQPENNYKTTPPVLDSDGSFFLYSKLTVDKSRWQQGN |
| | VFSCSVMHEALHNHYTQKSLSLSPGK, |
| | wherein the terminal lysine |
| | residue is present or absent (SEQ ID NO: 264) |
| Light chain | DIQMTQSPSSVSASVGDRVTITCRASQGISGWLAWYQQKPGKAPKFL |
| | IYAASTLQSGVPSRFSGSGSGTDFTLTISSLQPEDFATYYCQQANSFPP |
| | TFGGGTKVEIKRTVAAPSVFIFPPSDEQLKSGTASVVCLLNNFYPREA |
| | KVQWKVDNALQSGNSQESVTEQDSKDSTYSLSSTLTLSKADYEKHK |
| | VYACEVTHQGLSSPVTKSFNRGEC (SEQ ID NO: 265) |

In some embodiments, a heavy chain variable region of the anti-Nectin-4 antibody or fragment thereof comprises: (i) a HCDR1 comprising or consisting of the amino acid sequence of SEQ ID NO: 250; (ii) a HCDR2 comprising or consisting of the amino acid sequence of SEQ ID NO: 251; and (iii) a HCDR3 comprising or consisting of the amino acid sequence of SEQ ID NO: 252; and a light chain variable region of the anti-Nectin-4 antibody or fragment thereof comprises: (i) a LCDR1 comprising or consisting of the amino acid sequence of SEQ ID NO: 253; (ii) a LCDR2 comprising or consisting of the amino acid sequence of SEQ ID NO: 254; and (iii) a LCDR3 comprising or consisting of the amino acid sequence of SEQ ID NO: 255, wherein the CDRs are defined according to the Kabat numbering system.

In some embodiments, a heavy chain variable region of the anti-Nectin-4 antibody or fragment thereof comprises: (i) a HCDR1 comprising or consisting of the amino acid sequence of SEQ ID NO: 256; (ii) a HCDR2 comprising or consisting of the amino acid sequence of SEQ ID NO: 257; and (iii) a HCDR3 comprising or consisting of the amino acid sequence of SEQ ID NO: 258; and a light chain variable region of the anti-Nectin-4 antibody or fragment thereof comprises: (i) a LCDR1 comprising or consisting of the amino acid sequence of SEQ ID NO: 259; (ii) a LCDR2 comprising or consisting of the amino acid sequence of SEQ ID NO: 260; and (iii) a LCDR3 comprising or consisting of the amino acid sequence of SEQ ID NO: 261, wherein the CDRs are defined according to the IMGT numbering system.

In some embodiments, a heavy chain variable region of the anti-Nectin-4 antibody or fragment thereof comprises or consists of the amino acid sequence of SEQ ID NO: 262, or an amino acid sequence having at least 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, and a light chain variable region of the anti-Nectin-4 antibody or fragment thereof comprises or consists of the amino acid sequence of SEQ ID NO: 263 or an amino acid sequence having at least 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof.

In some embodiments, a heavy chain variable region of the anti-Nectin-4 antibody or fragment thereof comprises or consists of the amino acid sequence of SEQ ID NO: 262; and a light chain variable region of the anti-Nectin-4 antibody or fragment thereof comprises or consists of the amino acid sequence of SEQ ID NO: 263. In some embodiments, a heavy chain variable region of the anti-Nectin-4 antibody or fragment thereof consists of the amino acid sequence of SEQ ID NO: 262; and a light chain variable region of the anti-Nectin-4 antibody or fragment thereof consists of the amino acid sequence of SEQ ID NO: 263.

In some embodiments, a heavy chain of the anti-Nectin-4 antibody or fragment thereof comprises or consists of the amino acid sequence of the amino acid sequence of SEQ ID NO: 264, or an amino acid sequence having at least 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, and a light chain of the anti-Nectin-4 antibody or fragment thereof comprises or consists of the amino acid sequence of SEQ ID NO: 265 or an amino acid sequence having at least 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof.

In some embodiments, a heavy chain of the anti-Nectin-4 antibody or fragment thereof comprises or consists of the amino acid sequence of SEQ ID NO: 264; and a light chain of the anti-Nectin-4 antibody or fragment thereof comprises or consists of the amino acid sequence of SEQ ID NO: 265. In some embodiments, a heavy chain of the anti-Nectin-4 antibody or fragment thereof consists of the amino acid sequence of SEQ ID NO: 264; and a light chain of the anti-Nectin-4 antibody or fragment thereof consists of the amino acid sequence of SEQ ID NO: 265.

In some embodiments, the anti-Nectin-4 antibody or fragment thereof comprises or consists of two heavy chains comprising or consisting of the amino acid sequence of SEQ ID NO: 264; and two light chain comprising or consisting of the amino acid sequence of SEQ ID NO: 265. In some embodiments, the anti-Nectin-4 antibody or fragment thereof comprises or consists of two heavy chains consisting of the amino acid sequence of SEQ ID NO: 264; and two light chains consisting of the amino acid sequence of SEQ ID NO: 265.

In some embodiments, the anti-Nectin-4 antibody or fragment thereof is Enfortumab or a fragment thereof. In some embodiments, the anti-Nectin-4 antibody or fragment thereof is Enfortumab.

### Anti-TF Antibodies and Fragments Thereof.

In some embodiments, the target expressed on the surface of a tumour cell is Tissue factor (TF). TF is a protein that in humans is encoded by the F3 gene and which may also be known as platelet tissue factor or Coagulation factor III.

In some embodiments, the antibody-drug conjugate comprises an anti-TF antibody or fragment thereof.

The anti-TF antibody or fragment thereof may specifically bind TF. The anti-TF antibody may be a monoclonal antibody. In some embodiments, the anti-TF antibody or fragment thereof is a human antibody. In some embodiments, the anti-TF antibody or fragment thereof is a monoclonal human antibody. The fragment thereof may be any antigen-binding fragment thereof, for example a Fab, a Fab′, a F(ab′)2, a Fv, an ScFv, a Fab′-SH, an sdAb, or a VHH. In some embodiments, the anti-TF antibody or fragment thereof is a full-length anti-TF antibody.

The anti-TF antibody or fragment thereof may comprise a variable region that specifically binds TF. In some embodiments, the anti-TF antibody or fragment thereof comprises a heavy chain variable region and/or a light chain variable domain. In some embodiments, the anti-TF antibody or fragment thereof comprises a heavy chain variable region and a light chain variable domain. In some embodiments, the anti-TF antibody or fragment thereof comprises a constant region, preferably derived from a human antibody, preferably the constant region is selected from the constant region of human IgG1, IgG2, IgG3 or IgG4 (e.g. IgG1). In some embodiments, the anti-TF antibody or fragment thereof comprises a heavy chain and/or a light chain. In some embodiments, the anti-TF antibody or fragment thereof comprises a heavy chain and a light chain. In some embodiments, the anti-TF antibody or fragment thereof comprises or consists of two heavy chains and two light chains.

Example anti-TF antibody CDRs, variable region sequences, and heavy and light chain sequences are provided below.

| | |
|---|---|
| Kabat HCDR1 | NYAMS (SEQ ID NO: 266) |
| Kabat HCDR2 | SISGSGDYTYYTDSVKG (SEQ ID NO: 267) |
| Kabat HCDR3 | SPWGYYLDS (SEQ ID NO: 268) |
| Kabat LCDR1 | RASQGISSRLA (SEQ ID NO: 269) |
| Kabat LCDR2 | AASSLOS (SEQ ID NO: 270) |
| Kabat LCDR3 | QQYNSYPYT (SEQ ID NO: 271) |
| IMGT HCDR1 | GFTFSNYA (SEQ ID NO: 272) |
| IMGT HCDR2 | ISGSGDYT (SEQ ID NO: 273) |
| IMGT HCDR3 | ARSPWGYYLDS (SEQ ID NO: 274) |
| IMGT LCDRI | QGISSR (SEQ ID NO: 275) |
| IMGT LCDR2 | AAS (SEQ ID NO: 276) |
| IMGT LCDR3 | QQYNSYPYT (SEQ ID NO: 277) |
| VH | EVQLLESGGGLVQPGGSLRLSCAASGFTFSNYAMSWVRQAPGKGLE |
| | WVSSISGSGDYTYYTDSVKGRFTISRDNSKNTLYLQMNSLRAEDTAV |
| | YYCARSPWGYYLDSWGQGTLVTVSS (SEQ ID NO: 278) |
| VL | DIQMTQSPPSLSASAGDRVTITCRASQGISSRLAWYQQKPEKAPKSLI |
| | YAASSLQSGVPSRFSGSGSGTDFTLTISSLQPEDFATYYCQQYNSYPY |
| | TFGQGTKLEIK (SEQ ID NO: 279) |
| Heavy chain | EVQLLESGGGLVQPGGSLRLSCAASGFTFSNYAMSWVRQAPGKGLE |
| | WVSSISGSGDYTYYTDSVKGRFTISRDNSKNTLYLQMNSLRAEDTAV |
| | YYCARSPWGYYLDSWGQGTLVTVSSASTKGPSVFPLAPSSKSTSGGT |
| | AALGCLVKDYFPEPVTVSWNSGALTSGVHTFPAVLQSSGLYSLSSVV |
| | TVPSSSLGTQTYICNVNHKPSNTKVDKRVEPKSCDKTHTCPPCPAPEL |
| | LGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNK |
| | ALPAPIEKTISKAKGQPREPQVYTLPPSREEMTKNQVSLTCLVKGFYP |
| | SDIAVEWESNGQPENNYKTTPPVLDSDGSFFLYSKLTVDKSRWQQG |
| | NVFSCSVMHEALHNHYTQKSLSLSPGK (SEQ ID NO: 280) |
| Light chain | DIQMTQSPPSLSASAGDRVTITCRASQGISSRLAWYQQKPEKAPKSLI |
| | YAASSLQSGVPSRFSGSGSGTDFTLTISSLQPEDFATYYCQQYNSYPY |
| | TFGQGTKLEIKRTVAAPSVFIFPPSDEQLKSGTASVVCLLNNFYPREA |
| | KVQWKVDNALQSGNSQESVTEQDSKDSTYSLSSTLTLSKADYEKHK |
| | VYACEVTHQGLSSPVTKSFNRGEC (SEQ ID NO: 281) |

In some embodiments, a heavy chain variable region of the anti-TF antibody or fragment thereof comprises: (i) a HCDR1 comprising or consisting of the amino acid sequence of SEQ ID NO: 266; (ii) a HCDR2 comprising or consisting of the amino acid sequence of SEQ ID NO: 267; and (iii) a HCDR3 comprising or consisting of the amino acid sequence of SEQ ID NO: 268; and a light chain variable region of the anti-TF antibody or fragment thereof comprises: (i) a LCDR1 comprising or consisting of the amino acid sequence of SEQ ID NO: 269; (ii) a LCDR2 comprising or consisting of the amino acid sequence of SEQ ID NO: 270; and (iii) a LCDR3 comprising or consisting of the amino acid sequence of SEQ ID NO: 271, wherein the CDRs are defined according to the Kabat numbering system.

In some embodiments, a heavy chain variable region of the anti-TF antibody or fragment thereof comprises: (i) a HCDR1 comprising or consisting of the amino acid sequence of SEQ ID NO: 272; (ii) a HCDR2 comprising or consisting of the amino acid sequence of SEQ ID NO: 273; and (iii) a HCDR3 comprising or consisting of the amino acid sequence of SEQ ID NO: 274; and a light chain variable region of the anti-TF antibody or fragment thereof comprises: (i) a LCDR1 comprising or consisting of the amino acid sequence of SEQ ID NO: 275; (ii) a LCDR2 comprising or consisting of the amino acid sequence of SEQ ID NO: 276; and (iii) a LCDR3 comprising or consisting of the amino acid sequence of SEQ ID NO: 277, wherein the CDRs are defined according to the IMGT numbering system.

In some embodiments, a heavy chain variable region of the anti-TF antibody or fragment thereof comprises or consists of the amino acid sequence of SEQ ID NO: 278, or an amino acid sequence having at least 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, and a light chain variable region of the anti-TF antibody or fragment thereof comprises or consists of the amino acid sequence of SEQ ID NO: 279 or an amino acid sequence having at least 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof.

In some embodiments, a heavy chain variable region of the anti-TF antibody or fragment thereof comprises or consists of the amino acid sequence of SEQ ID NO: 278; and a light chain variable region of the anti-TF antibody or fragment thereof comprises or consists of the amino acid sequence of SEQ ID NO: 279. In some embodiments, a heavy chain variable region of the anti-TF antibody or fragment thereof consists of the amino acid sequence of SEQ ID NO: 278; and a light chain variable region of the anti-TF antibody or fragment thereof consists of the amino acid sequence of SEQ ID NO: 279.

In some embodiments, a heavy chain of the anti-TF antibody or fragment thereof comprises or consists of the amino acid sequence of the amino acid sequence of SEQ ID NO: 280, or an amino acid sequence having at least 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, and a light chain of the anti-TF antibody or fragment thereof comprises or consists of the amino acid sequence of SEQ ID NO: 281 or an amino acid sequence having at least 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof.

In some embodiments, a heavy chain of the anti-TF antibody or fragment thereof comprises or consists of the amino acid sequence of SEQ ID NO: 280; and a light chain of the anti-TF antibody or fragment thereof comprises or consists of the amino acid sequence of SEQ ID NO: 281. In some embodiments, a heavy chain of the anti-TF antibody or fragment thereof consists of the amino acid sequence of SEQ ID NO: 280; and a light chain of the anti-TF antibody or fragment thereof consists of the amino acid sequence of SEQ ID NO: 281.

In some embodiments, the anti-TF antibody or fragment thereof comprises or consists of two heavy chains comprising or consisting of the amino acid sequence of SEQ ID NO: 280; and two light chain comprising or consisting of the amino acid sequence of SEQ ID NO: 281. In some embodiments, the anti-TF antibody or fragment thereof comprises or consists of two heavy chains consisting of the amino acid sequence of SEQ ID NO: 280; and two light chains consisting of the amino acid sequence of SEQ ID NO: 281.

In some embodiments, the anti-TF antibody or fragment thereof is Tisotumab or a fragment thereof. In some embodiments, the anti-TF antibody or fragment thereof is Tisotumab.

### Anti-B7-H4 Antibodies and Fragments Thereof.

In some embodiments, the target expressed on the surface of a tumour cell is B7-H4. B7-H4 is a protein that in humans is encoded by the VTCN1 gene and which may also be known as V-Set Domain Containing T Cell Activation Inhibitor 1.

In some embodiments, the antibody-drug conjugate comprises an anti-B7-H4 antibody or fragment thereof.

The anti-B7-H4 antibody or fragment thereof may specifically bind B7-H4. The anti-B7-H4 antibody may be a monoclonal antibody. In some embodiments, the anti-B7-H4 antibody or fragment thereof is a human antibody. In some embodiments, the anti-B7-H4 antibody or fragment thereof is a monoclonal human antibody. The fragment thereof may be any antigen-binding fragment thereof, for example a Fab, a Fab′, a F(ab′)2, a Fv, an ScFv, a Fab′-SH, an sdAb, or a VHH. In some embodiments, the anti-B7-H4 antibody or fragment thereof is a full-length anti-B7-H4 antibody.

The anti-B7-H4 antibody or fragment thereof may comprise a variable region that specifically binds B7-H4. In some embodiments, the anti-B7-H4 antibody or fragment thereof comprises a heavy chain variable region and/or a light chain variable domain. In some embodiments, the anti-B7-H4 antibody or fragment thereof comprises a heavy chain variable region and a light chain variable domain. In some embodiments, the anti-B7-H4 antibody or fragment thereof comprises a constant region, preferably derived from a human antibody, preferably the constant region is selected from the constant region of human IgG1, IgG2, IgG3 or IgG4 (e.g. IgG1). In some embodiments, the anti-B7-H4 antibody or fragment thereof comprises a heavy chain and/or a light chain. In some embodiments, the anti-B7-H4 antibody or fragment thereof comprises a heavy chain and a light chain. In some embodiments, the anti-B7-H4 antibody or fragment thereof comprises or consists of two heavy chains and two light chains.

Example anti-B7-H4 antibody CDRs, variable region sequences, and heavy and light chain sequences are provided below.

| | |
|---|---|
| Kabat HCDR1 | SGSYYWG (SEQ ID NO: 282) |
| Kabat HCDR2 | NIYYSGSTYYNPSLRS (SEQ ID NO: 283) |
| Kabat HCDR3 | EGSYPNQFDP (SEQ ID NO: 284) |
| Kabat LCDR1 | RASQSVSSNLA (SEQ ID NO: 285) |
| Kabat LCDR2 | GASTRAT (SEQ ID NO: 286) |
| Kabat LCDR3 | QQYHSFPFT (SEQ ID NO: 287) |
| IMGT HCDR1 | GGSIKSGSYY (SEQ ID NO: 288) |
| IMGT HCDR2 | IYYSGST (SEQ ID NO: 289) |
| IMGT HCDR3 | AREGSYPNQFDP (SEQ ID NO: 290) |
| IMGT LCDR1 | QSVSSN (SEQ ID NO: 291) |
| IMGT LCDR2 | GAS (SEQ ID NO: 292) |
| IMGT LCDR3 | QQYHSFPFT (SEQ ID NO: 293) |
| VH | QLQLQESGPGLVKPSETLSLTCTVSGGSIKSGSYYWGWIRQPPGKGL |
| | EWIGNIYYSGSTYYNPSLRSRVTISVDTSKNQFSLKLSSVTAADTAVY |
| | YCAREGSYPNQFDPWGQGTLVTVSS (SEQ ID NO: 294) |
| VL | EIVMTQSPATLSVSPGERATLSCRASQSVSSNLAWYQQKPGQAPRLLI |
| | YGASTRATGIPARFSGSGSGTEFTLTISSLQSEDFAVYYCQQYHSFPFT |
| | FGGGTKVEIK (SEQ ID NO: 295) |
| Heavy chain | QLQLQESGPGLVKPSETLSLTCTVSGGSIKSGSYYWGWIRQPPGKGL |
| | EWIGNIYYSGSTYYNPSLRSRVTISVDTSKNQFSLKLSSVTAADTAVY |
| | YCAREGSYPNQFDPWGQGTLVTVSSASTKGPSVFPLAPSSKSTSGGT |
| | AALGCLVKDYFPEPVTVSWNSGALTSGVHTFPAVLQSSGLYSLSSVV |
| | TVPSSSLGTQTYICNVNHKPSNTKVDKKVEPKSCDKTHTCPPCPAPEL |
| | LGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNK |
| | ALPAPIEKTISKAKGQPREPQVYTLPPSRDELTKNQVSLTCLVKGFYP |
| | SDIAVEWESNGQPENNYKTTPPVLDSDGSFFLYSKLTVDKSRWQQG |
| | NVFSCSVMHEALHNHYTQKSLSLSPGK (SEQ ID NO: 296) |
| Light chain | EIVMTQSPATLSVSPGERATLSCRASQSVSSNLAWYQQKPGQAPRLLI |
| | YGASTRATGIPARFSGSGSGTEFTLTISSLQSEDFAVYYCQQYHSFPFT |
| | FGGGTKVEIKRTVAAPSVFIFPPSDEQLKSGTASVVCLLNNFYPREAK |
| | VQWKVDNALQSGNSQESVTEQDSKDSTYSLSSTLTLSKADYEKHKV |
| | YACEVTHQGLSSPVTKSFNRGEC (SEQ ID NO: 297) |

In some embodiments, a heavy chain variable region of the anti-B7-H4 antibody or fragment thereof comprises: (i) a HCDR1 comprising or consisting of the amino acid sequence of SEQ ID NO: 282; (ii) a HCDR2 comprising or consisting of the amino acid sequence of SEQ ID NO: 283; and (iii) a HCDR3 comprising or consisting of the amino acid sequence of SEQ ID NO: 284; and a light chain variable region of the anti-B7-H4 antibody or fragment thereof comprises: (i) a LCDR1 comprising or consisting of the amino acid sequence of SEQ ID NO: 285; (ii) a LCDR2 comprising or consisting of the amino acid sequence of SEQ ID NO: 286; and (iii) a LCDR3 comprising or consisting of the amino acid sequence of SEQ ID NO: 287, wherein the CDRs are defined according to the Kabat numbering system.

In some embodiments, a heavy chain variable region of the anti-B7-H4 antibody or fragment thereof comprises: (i) a HCDR1 comprising or consisting of the amino acid sequence of SEQ ID NO: 288; (ii) a HCDR2 comprising or consisting of the amino acid sequence of SEQ ID NO: 289; and (iii) a HCDR3 comprising or consisting of the amino acid sequence of SEQ ID NO: 290; and a light chain variable region of the anti-B7-H4 antibody or fragment thereof comprises: (i) a LCDR1 comprising or consisting of the amino acid sequence of SEQ ID NO: 291; (ii) a LCDR2 comprising or consisting of the amino acid sequence of SEQ ID NO: 292; and (iii) a LCDR3 comprising or consisting of the amino acid sequence of SEQ ID NO: 293, wherein the CDRs are defined according to the IMGT numbering system.

In some embodiments, a heavy chain variable region of the anti-B7-H4 antibody or fragment thereof comprises or consists of the amino acid sequence of SEQ ID NO: 294, or an amino acid sequence having at least 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof; and a light chain variable region of the anti-B7-H4 antibody or fragment thereof comprises or consists of the amino acid sequence of SEQ ID NO: 295 or an amino acid sequence having at least 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof.

In some embodiments, a heavy chain variable region of the anti-B7-H4 antibody or fragment thereof comprises or consists of the amino acid sequence of SEQ ID NO: 294; and a light chain variable region of the anti-B7-H4 antibody or fragment thereof comprises or consists of the amino acid sequence of SEQ ID NO: 295. In some embodiments, a heavy chain variable region of the anti-B7-H4 antibody or fragment thereof consists of the amino acid sequence of SEQ ID NO: 294; and a light chain variable region of the anti-B7-H4 antibody or fragment thereof consists of the amino acid sequence of SEQ ID NO: 295.

In some embodiments, a heavy chain of the anti-B7-H4 antibody or fragment thereof comprises or consists of the amino acid sequence of the amino acid sequence of SEQ ID NO: 296, or an amino acid sequence having at least 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof; and a light chain of the anti-B7-H4 antibody or fragment thereof comprises or consists of the amino acid sequence of SEQ ID NO: 297 or an amino acid sequence having at least 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof.

In some embodiments, a heavy chain of the anti-B7-H4 antibody or fragment thereof comprises or consists of the amino acid sequence of SEQ ID NO: 296; and a light chain of the anti-B7-H4 antibody or fragment thereof comprises or consists of the amino acid sequence of SEQ ID NO: 297. In some embodiments, a heavy chain of the anti-B7-H4 antibody or fragment thereof consists of the amino acid sequence of SEQ ID NO: 296; and a light chain of the anti-B7-H4 antibody or fragment thereof consists of the amino acid sequence of SEQ ID NO: 297.

In some embodiments, the anti-B7-H4 antibody or fragment thereof comprises or consists of two heavy chains comprising or consisting of the amino acid sequence of SEQ ID NO: 296; and two light chain comprising or consisting of the amino acid sequence of SEQ ID NO: 297. In some embodiments, the anti-B7-H4 antibody or fragment thereof comprises or consists of two heavy chains consisting of the amino acid sequence of SEQ ID NO: 296; and two light chains consisting of the amino acid sequence of SEQ ID NO: 297.

In some embodiments, the anti-B7-H4 antibody or fragment thereof is Felmetatug or a fragment thereof. In some embodiments, the anti-B7-H4 antibody or fragment thereof is Felmetatug.

In some embodiments, the anti-B7-H4 antibody or fragment thereof is Alsevalimab or a fragment thereof. In some embodiments, the anti-B7-H4 antibody or fragment thereof is Alsevalimab.

### Anti-ITGB6 Antibodies and Fragments Thereof.

In some embodiments, the target expressed on the surface of a tumour cell is integrin beta-6 (ITGB6). ITGB6 is a protein that in humans is encoded by the ITGB6 gene.

In some embodiments, the antibody-drug conjugate comprises an anti-ITGB6 antibody or fragment thereof.

The anti-ITGB6 antibody or fragment thereof may specifically bind ITGB6. The anti-ITGB6 antibody may be a monoclonal antibody. In some embodiments, the anti-ITGB6 antibody or fragment thereof is a human antibody. In some embodiments, the anti-ITGB6 antibody or fragment thereof is a monoclonal human antibody. The fragment thereof may be any antigen-binding fragment thereof, for example a Fab, a Fab′, a F(ab′)2, a Fv, an ScFv, a Fab′-SH, an sdAb, or a VHH. In some embodiments, the anti-ITGB6 antibody or fragment thereof is a full-length anti-ITGB6 antibody.

The anti-ITGB6 antibody or fragment thereof may comprise a variable region that specifically binds ITGB6. In some embodiments, the anti-ITGB6 antibody or fragment thereof comprises a heavy chain variable region and/or a light chain variable domain. In some embodiments, the anti-ITGB6 antibody or fragment thereof comprises a heavy chain variable region and a light chain variable domain. In some embodiments, the anti-ITGB6 antibody or fragment thereof comprises a constant region, preferably derived from a human antibody, preferably the constant region is selected from the constant region of human IgG1, IgG2, IgG3 or IgG4 (e.g. IgG1). In some embodiments, the anti-ITGB6 antibody or fragment thereof comprises a heavy chain and/or a light chain. In some embodiments, the anti-ITGB6 antibody or fragment thereof comprises a heavy chain and a light chain. In some embodiments, the anti-ITGB6 antibody or fragment thereof comprises or consists of two heavy chains and two light chains. Example anti-ITGB6 antibody CDRs, variable region sequences, and heavy and light chain sequences are provided below.

| | |
|---|---|
| Kabat HCDR1 | DYNVN (SEQ ID NO: 298) |
| Kabat HCDR2 | VINPKYGTTRYNQKFKG (SEQ ID NO: 299) |
| Kabat HCDR3 | GLNAWDY (SEQ ID NO: 300) |
| Kabat LCDR1 | GASENIYGALN (SEQ ID NO: 301) |
| Kabat LCDR2 | GATNLED (SEQ ID NO: 302) |
| Kabat LCDR3 | QNVLTTPYT (SEQ ID NO: 303) |
| IMGT HCDR1 | GYSFTDYN (SEQ ID NO: 304) |
| IMGT HCDR2 | INPKYGTT (SEQ ID NO: 305) |
| IMGT HCDR3 | TRGLNAWDY (SEQ ID NO: 306) |
| IMGT LCDR1 | ENIYGA (SEQ ID NO: 307) |
| IMGT LCDR2 | GAT (SEQ ID NO: 308) |
| IMGT LCDR3 | QNVLTTPYT (SEQ ID NO: 309) |
| VH | QFQLVQSGAEVKKPGASVKVSCKASGYSFTDYNVNWVRQAPGQGL |
| | EWIGVINPKYGTTRYNQKFKGRATLTVDKSTSTAYMELSSLRSEDTA |
| | VYYCTRGLNAWDYWGQGTLVTVSS (SEQ ID NO: 310) |
| VL | DIQMTQSPSSLSASVGDRVTITCGASENIYGALNWYQQKPGKAPKLL |
| | IYGATNLEDGVPSRFSGSGSGRDYTFTISSLQPEDIATYYCQNVLTTP |
| | YTFGQGTKLEIK (SEQ ID NO: 311) |
| Heavy chain | QFQLVQSGAEVKKPGASVKVSCKASGYSFTDYNVNWVRQAPGQGL |
| | EWIGVINPKYGTTRYNQKFKGRATLTVDKSTSTAYMELSSLRSEDTA |
| | VYYCTRGLNAWDYWGQGTLVTVSSASTKGPSVFPLAPSSKSTSGGT |
| | AALGCLVKDYFPEPVTVSWNSGALTSGVHTFPAVLQSSGLYSLSSVV |
| | TVPSSSLGTQTYICNVNHKPSNTKVDKKVEPKSCDKTHTCPPCPAPEL |
| | LGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNK |
| | ALPAPIEKTISKAKGQPREPQVYTLPPSRDELTKNQVSLTCLVKGFYP |
| | SDIAVEWESNGQPENNYKTTPPVLDSDGSFFLYSKLTVDKSRWQQG |
| | NVFSCSVMHEALHNHYTQKSLSLSPGK (SEQ ID NO: 312) |
| Light chain | DIQMTQSPSSLSASVGDRVTITCGASENIYGALNWYQQKPGKAPKLL |
| | IYGATNLEDGVPSRFSGSGSGRDYTFTISSLQPEDIATYYCQNVLTTP |
| | YTFGQGTKLEIKRTVAAPSVFIFPPSDEQLKSGTASVVCLLNNFYPRE |
| | AKVQWKVDNALQSGNSQESVTEQDSKDSTYSLSSTLTLSKADYEKH |
| | KVYACEVTHQGLSSPVTKSFNRGEC (SEQ ID NO: 313) |

In some embodiments, a heavy chain variable region of the anti-ITGB6 antibody or fragment thereof comprises: (i) a HCDR1 comprising or consisting of the amino acid sequence of SEQ ID NO: 298; (ii) a HCDR2 comprising or consisting of the amino acid sequence of SEQ ID NO: 299; and (iii) a HCDR3 comprising or consisting of the amino acid sequence of SEQ ID NO: 300; and a light chain variable region of the anti-ITGB6 antibody or fragment thereof comprises: (i) a LCDR1 comprising or consisting of the amino acid sequence of SEQ ID NO: 301; (ii) a LCDR2 comprising or consisting of the amino acid sequence of SEQ ID NO: 302; and (iii) a LCDR3 comprising or consisting of the amino acid sequence of SEQ ID NO: 303, wherein the CDRs are defined according to the Kabat numbering system.

In some embodiments, a heavy chain variable region of the anti-ITGB6 antibody or fragment thereof comprises: (i) a HCDR1 comprising or consisting of the amino acid sequence of SEQ ID NO: 304; (ii) a HCDR2 comprising or consisting of the amino acid sequence of SEQ ID NO: 305; and (iii) a HCDR3 comprising or consisting of the amino acid sequence of SEQ ID NO: 306; and a light chain variable region of the anti-ITGB6 antibody or fragment thereof comprises: (i) a LCDR1 comprising or consisting of the amino acid sequence of SEQ ID NO: 307; (ii) a LCDR2 comprising or consisting of the amino acid sequence of SEQ ID NO: 308; and (iii) a LCDR3 comprising or consisting of the amino acid sequence of SEQ ID NO: 309, wherein the CDRs are defined according to the IMGT numbering system.

In some embodiments, a heavy chain variable region of the anti-ITGB6 antibody or fragment thereof comprises or consists of the amino acid sequence of SEQ ID NO: 310, or an amino acid sequence having at least 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof; and a light chain variable region of the anti-ITGB6 antibody or fragment thereof comprises or consists of the amino acid sequence of SEQ ID NO: 311 or an amino acid sequence having at least 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof.

In some embodiments, a heavy chain variable region of the anti-ITGB6 antibody or fragment thereof comprises or consists of the amino acid sequence of SEQ ID NO: 310; and a light chain variable region of the anti-ITGB6 antibody or fragment thereof comprises or consists of the amino acid sequence of SEQ ID NO: 311. In some embodiments, a heavy chain variable region of the anti-ITGB6 antibody or fragment thereof consists of the amino acid sequence of SEQ ID NO: 310; and a light chain variable region of the anti-ITGB6 antibody or fragment thereof consists of the amino acid sequence of SEQ ID NO: 311.

In some embodiments, a heavy chain of the anti-ITGB6 antibody or fragment thereof comprises or consists of the amino acid sequence of the amino acid sequence of SEQ ID NO: 312, or an amino acid sequence having at least 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof; and a light chain of the anti-ITGB6 antibody or fragment thereof comprises or consists of the amino acid sequence of SEQ ID NO: 313 or an amino acid sequence having at least 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof.

In some embodiments, a heavy chain of the anti-ITGB6 antibody or fragment thereof comprises or consists of the amino acid sequence of SEQ ID NO: 312; and a light chain of the anti-ITGB6 antibody or fragment thereof comprises or consists of the amino acid sequence of SEQ ID NO: 313. In some embodiments, a heavy chain of the anti-ITGB6 antibody or fragment thereof consists of the amino acid sequence of SEQ ID NO: 312; and a light chain of the anti-ITGB6 antibody or fragment thereof consists of the amino acid sequence of SEQ ID NO: 313.

In some embodiments, the anti-ITGB6 antibody or fragment thereof comprises or consists of two heavy chains comprising or consisting of the amino acid sequence of SEQ ID NO: 312; and two light chain comprising or consisting of the amino acid sequence of SEQ ID NO: 313.

In some embodiments, the anti-ITGB6 antibody or fragment thereof comprises or consists of two heavy chains consisting of the amino acid sequence of SEQ ID NO: 312; and two light chains consisting of the amino acid sequence of SEQ ID NO: 313.

In some embodiments, the anti-ITGB6 antibody or fragment thereof is Sigvotatug or a fragment thereof. In some embodiments, the anti-ITGB6 antibody or fragment thereof is Sigvotatug.

### Anti-HER2 Antibodies and Fragments Thereof.

In some embodiments, the target expressed on the surface of a tumour cell is HER2. In some embodiments, the antibody-drug conjugate comprises an anti-HER2 antibody or fragment thereof.

The anti-HER2 antibody or fragment thereof may specifically bind HER2. The anti-HER2 antibody may be a monoclonal antibody. In some embodiments, the anti-HER2 antibody or fragment thereof is a humanized antibody. In some embodiments, the anti-HER2 antibody or fragment thereof is a monoclonal humanized antibody. The fragment thereof may be any antigen-binding fragment thereof, for example a Fab, a Fab′, a F(ab′)2, a Fv, an ScFv, a Fab′-SH, an sdAb, or a VHH. In some embodiments, the anti-HER2 antibody or fragment thereof is a full-length anti-HER2 antibody.

The anti-HER2 antibody or fragment thereof may comprise a variable region that specifically binds HER2. In some embodiments, the anti-HER2 antibody or fragment thereof comprises a heavy chain variable region and/or a light chain variable domain. In some embodiments, the anti-HER2 antibody or fragment thereof comprises a heavy chain variable region and a light chain variable domain. In some embodiments, the anti-HER2 antibody or fragment thereof comprises a constant region, preferably derived from a human antibody, preferably the constant region is selected from the constant region of human IgG1, IgG2, IgG3 or IgG4 (e.g. IgG1). In some embodiments, the anti-HER2 antibody or fragment thereof comprises a heavy chain and/or a light chain. In some embodiments, the anti-HER2 antibody or fragment thereof comprises a heavy chain and a light chain. In some embodiments, the anti-HER2 antibody or fragment thereof comprises or consists of two heavy chains and two light chains.

Example anti-HER2 antibody CDRs, variable region sequences, and heavy and light chain sequences are provided below.

| | |
|---|---|
| Kabat HCDR1 | DYYIH (SEQ ID NO: 314) |
| Kabat HCDR2 | RVNPDHGDSYYNQKFKD (SEQ ID NO: 315) |
| Kabat HCDR3 | NYLFDH (SEQ ID NO: 316) |
| Kabat LCDR1 | KASQDVGTAVA (SEQ ID NO: 317) |
| Kabat LCDR2 | WASIRHT (SEQ ID NO: 318) |
| Kabat LCDR3 | HQFATYT (SEQ ID NO: 319) |
| IMGT HCDR1 | GYTFTDYY (SEQ ID NO: 320) |
| IMGT HCDR2 | VNPDHGDS (SEQ ID NO: 321) |
| IMGT HCDR3 | ARNYLFDH (SEQ ID NO: 322) |
| IMGT LCDR1 | QDVGTA (SEQ ID NO: 323) |
| IMGT LCDR2 | WAS (SEQ ID NO: 324) |
| IMGT LCDR3 | HQFATYT (SEQ ID NO: 325) |
| VH | EVQLVQSGAEVKKPGATVKISCKVSGYTFTDYYIHWVQQAPGKGLE |
| | WMGRVNPDHGDSYYNQKFKDKATITADKSTDTAYMELSSLRSEDT |
| | AVYFCARNYLFDHWGQGTLVTVSS (SEQ ID NO: 326) |
| VL | DIQMTQSPSSVSASVGDRVTITCKASQDVGTAVAWYQQKPGKAPKL |
| | LIYWASIRHTGVPSRFSGSGSGTDFTLTISSLQPEDFATYYCHQFATYT |
| | FGGGTKVEIK (SEQ ID NO: 327) |
| Heavy chain | EVQLVQSGAEVKKPGATVKISCKVSGYTFTDYYIHWVQQAPGKGLE |
| | WMGRVNPDHGDSYYNQKFKDKATITADKSTDTAYMELSSLRSEDT |
| | AVYFCARNYLFDHWGQGTLVTVSSASTKGPSVFPLAPSSKSTSGGTA |
| | ALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPELL |
| | GGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKA |
| | LPAPIEKTISKAKGQPREPQVYTLPPSREEMTKNQVSLTCLVKGFYPS |
| | DIAVEWESNGQPENNYKTTPPVLDSDGSFFLYSKLTVDKSRWQQGN |
| | VFSCSVMHEALHNHYTQKSLSLSPGK (SEQ ID NO: 328) |
| Light chain | DIQMTQSPSSVSASVGDRVTITCKASQDVGTAVAWYQQKPGKAPKL |
| | LIYWASIRHTGVPSRFSGSGSGTDFTLTISSLQPEDFATYYCHQFATYT |
| | FGGGTKVEIKRTVAAPSVFIFPPSDEQLKSGTASVVCLLNNFYPREAK |
| | VQWKVDNALQSGNSQESVTEQDSKDSTYSLSSTLTLSKADYEKHKV |
| | YACEVTHQGLSSPVTKSFNRGEC (SEQ ID NO: 329) |

In some embodiments, a heavy chain variable region of the anti-HER2 antibody or fragment thereof comprises: (i) a HCDR1 comprising or consisting of the amino acid sequence of SEQ ID NO: 314; (ii) a HCDR2 comprising or consisting of the amino acid sequence of SEQ ID NO: 315; and (iii) a HCDR3 comprising or consisting of the amino acid sequence of SEQ ID NO: 316; and a light chain variable region of the anti-HER2 antibody or fragment thereof comprises: (i) a LCDR1 comprising or consisting of the amino acid sequence of SEQ ID NO: 317; (ii) a LCDR2 comprising or consisting of the amino acid sequence of SEQ ID NO: 318; and (iii) a LCDR3 comprising or consisting of the amino acid sequence of SEQ ID NO: 319, wherein the CDRs are defined according to the Kabat numbering system.

In some embodiments, a heavy chain variable region of the anti-HER2 antibody or fragment thereof comprises: (i) a HCDR1 comprising or consisting of the amino acid sequence of SEQ ID NO: 320; (ii) a HCDR2 comprising or consisting of the amino acid sequence of SEQ ID NO: 321; and (iii) a HCDR3 comprising or consisting of the amino acid sequence of SEQ ID NO: 322; and a light chain variable region of the anti-HER2 antibody or fragment thereof comprises: (i) a LCDR1 comprising or consisting of the amino acid sequence of SEQ ID NO: 323; (ii) a LCDR2 comprising or consisting of the amino acid sequence of SEQ ID NO: 324; and (iii) a LCDR3 comprising or consisting of the amino acid sequence of SEQ ID NO: 325, wherein the CDRs are defined according to the IMGT numbering system.

In some embodiments, a heavy chain variable region of the anti-HER2 antibody or fragment thereof comprises or consists of the amino acid sequence of SEQ ID NO: 326, or an amino acid sequence having at least 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof; and a light chain variable region of the anti-HER2 antibody or fragment thereof comprises or consists of the amino acid sequence of SEQ ID NO: 327 or an amino acid sequence having at least 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof.

In some embodiments, a heavy chain variable region of the anti-HER2 antibody or fragment thereof comprises or consists of the amino acid sequence of SEQ ID NO: 326; and a light chain variable region of the anti-HER2 antibody or fragment thereof comprises or consists of the amino acid sequence of SEQ ID NO: 327. In some embodiments, a heavy chain variable region of the anti-HER2 antibody or fragment thereof consists of the amino acid sequence of SEQ ID NO: 326; and a light chain variable region of the anti-HER2 antibody or fragment thereof consists of the amino acid sequence of SEQ ID NO: 327.

In some embodiments, a heavy chain of the anti-HER2 antibody or fragment thereof comprises or consists of the amino acid sequence of the amino acid sequence of SEQ ID NO: 328, or an amino acid sequence having at least 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof; and a light chain of the anti-HER2 antibody or fragment thereof comprises or consists of the amino acid sequence of SEQ ID NO: 329 or an amino acid sequence having at least 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof.

In some embodiments, a heavy chain of the anti-HER2 antibody or fragment thereof comprises or consists of the amino acid sequence of SEQ ID NO: 328; and a light chain of the anti-HER2 antibody or fragment thereof comprises or consists of the amino acid sequence of SEQ ID NO: 329. In some embodiments, a heavy chain of the anti-HER2 antibody or fragment thereof consists of the amino acid sequence of SEQ ID NO: 328; and a light chain of the anti-HER2 antibody or fragment thereof consists of the amino acid sequence of SEQ ID NO: 329.

In some embodiments, the anti-HER2 antibody or fragment thereof comprises or consists of two heavy chains comprising or consisting of the amino acid sequence of SEQ ID NO: 328; and two light chain comprising or consisting of the amino acid sequence of SEQ ID NO: 329. In some embodiments, the anti-HER2 antibody or fragment thereof comprises or consists of two heavy chains consisting of the amino acid sequence of SEQ ID NO: 328; and two light chains consisting of the amino acid sequence of SEQ ID NO: 329.

In some embodiments, the anti-HER2 antibody or fragment thereof is Disitamab or a fragment thereof. In some embodiments, the anti-HER2 antibody or fragment thereof is Disitamab.

### Linker and Payload Moieties

Typically, in the antibody-drug conjugates of the present invention, one or more payloads or payload moieties are linked, preferably covalently linked to the antibody or fragment thereof via a linker to form the antibody-drug conjugate. The moiety linked to the antibody or fragment thereof is generally referred to herein as the “linker-payload moiety”. The linker-payload moiety comprises a linker, as defined herein, and a payload, as defined herein.

Typically, when the antibody-drug conjugate is administered to the subject, the linker-payload moiety is released by breakdown of the antibody-drug conjugate in vivo. The linker-payload moiety then typically breaks down to release the payload in vivo.

The drug-antibody ratio (DAR) is the number of linker-payload molecules attached to each antibody or fragment thereof (it can be understood that the term “drug-antibody ratio” applies equally whether the linker-payload molecule is attached to an antibody or to a fragment thereof). As indicated above, the drug-antibody ratio (DAR) of the antibody-drug conjugates used in the combination of the invention may vary. In this specification the terms “drug-antibody ratio”, “DAR” and “connection number” are synonymous.

In some embodiments, the DAR is an integer from 1 to 16. In some embodiments, the DAR is an integer of 1, 2, 3, 4, 5, 6, 7, 8, 9, 10, 11, 12, 13, 14, 15 or 16. In some embodiments, the DAR is an integer from 4 to 12. In some embodiments, the DAR is an integer from 4 to 10.

In some embodiments, the DAR is an integer from 6 to 10. In some embodiments, the DAR is an integer from 3 to 9. In some embodiments, the DAR is an integer from 4 to 8. In some embodiments, the DAR is an integer from 5 to 7. In some embodiments, the DAR is an integer from 7 to 9. In some embodiments, the DAR is 6. In some embodiments, the DAR is 7. In some embodiments, the DAR is 8. In some embodiments, the DAR is 9. In some embodiments, the DAR is 10.

In some embodiments, the DAR is an integer from 2 to 8. In some embodiments, the DAR is an integer from 3 to 6. In some embodiments, the DAR is 4 or 5. In some embodiments, the DAR is 4. In some embodiments, the DAR is 5.

In some embodiments, the linker is a cleavable linker. In some embodiments, the linker is a linker cleavable by a lysosomal peptidase.

In some embodiments, the linker comprises a peptide linker. In some embodiments, the linker consists essentially of a peptide linker. In some embodiments, the linker consists of a peptide linker. Typically, the peptide linker comprises, essentially of or consist of 1 to 10 amino acid residues. The peptide linker may comprise, consist essentially of or consist of 1, 2, 3, 4, 5, 6, 7, 8, 9 or 10 amino acid residues. In some embodiments, the peptide linker comprises, consists essentially of or consists of 1 to 6 amino acid residues. In some embodiments, the peptide linker comprises, consists essentially of or consists of 1 to 5 amino acid residues. In some embodiments, the peptide linker comprises, consists essentially of or consists of 2 amino acid residues. In some embodiments, the peptide linker comprises, consists essentially of or consists of 3 amino acid residues. In some embodiments, the peptide linker comprises, consists essentially of or consists of 4 amino acid residues. In some embodiments, the peptide linker comprises, consists essentially of or consists of 5 amino acid residues.

In one embodiment, the linker is selected from the group consisting of AA1, AA1-Gly, Val-Cit, Val-Ala, Val-AA1, Val-AA1-Gly, AA1-Ala-Asn, Ala-Ala-Ala, Ala-Ala-Asn and Gly-Gly-Phe-Gly.

In one embodiment, the amino acid residue represented by AA1 is selected from the group consisting of

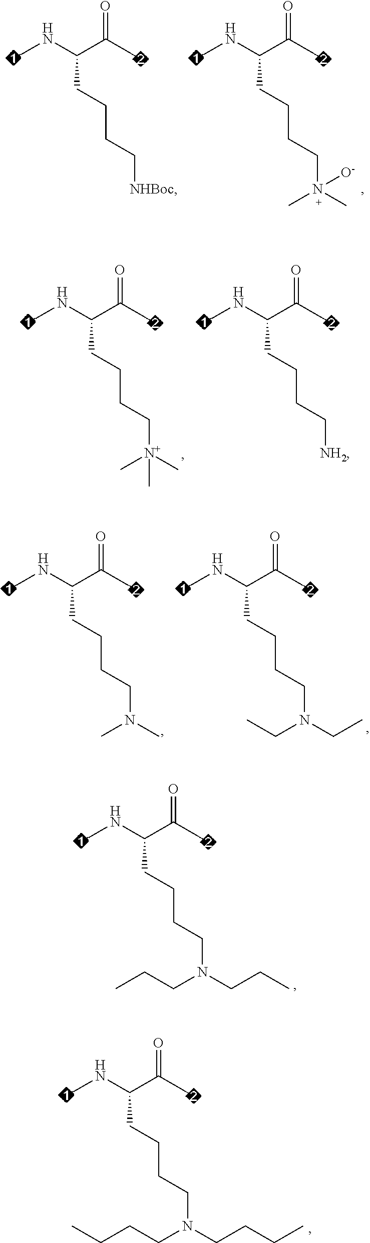

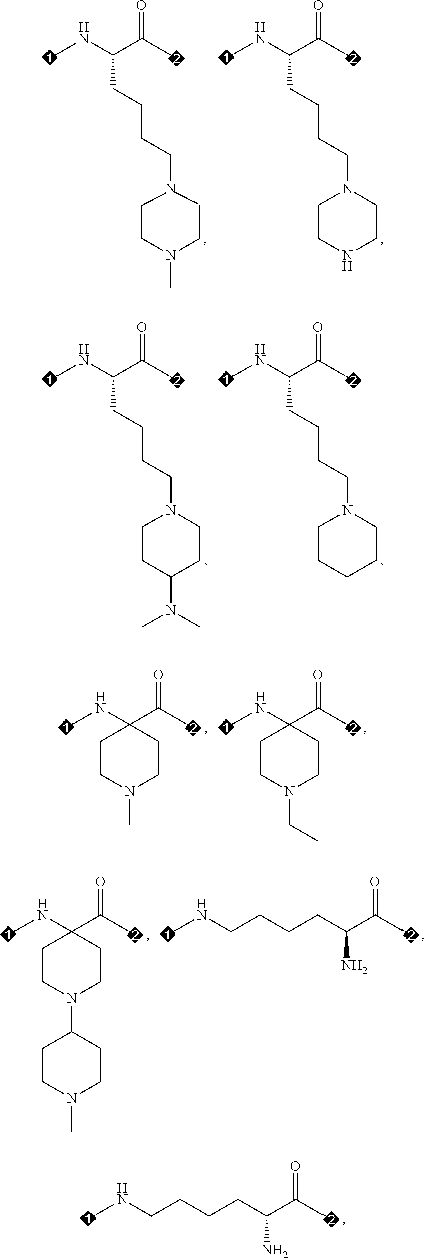

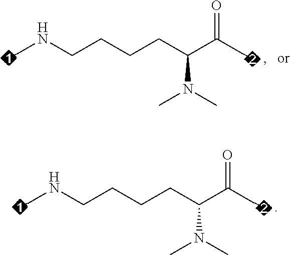

where position 1 is connected, optionally via a further conjugation moiety, to the portion of the molecule bearing the antibody or fragment thereof and position 2 is connected, optionally via a further conjugation moiety, to the portion of the molecule bearing the payload compound.

In some embodiments, the peptide linker comprises, consists essentially of, or consists of a Gly-Gly-Phe-Gly (GGFG) peptide moiety. In some embodiments, the peptide linker comprises a Gly-Gly-Phe-Gly (GGFG) peptide moiety.

In some embodiments, the peptide linker comprises, consists essentially of, or consists of a Val-Cit peptide moiety.

In some embodiments, the linker comprises a first conjugation moiety for coupling with the antibody or fragment thereof.

In one embodiment, the first conjugation moiety comprises a pyrimidine-sulfone moiety. In one embodiment, the pyrimidine-sulfone moiety is linked to the peptide moiety via a C1-10 alkynoyl group.

In one embodiment, the first conjugation moiety comprises a maleimide moiety. In one embodiment, the maleimide moiety is linked to the peptide moiety via a C1-10 alkanoyl group.

In one embodiment, the linker comprises a second conjugation moiety for coupling with the payload.

In one embodiment, the linker comprises a spacer moiety. In one embodiment, the linker comprises a peptide moiety and the spacer moiety is conjugated to the C-terminus of the peptide moiety. In one embodiment, the linker-payload moiety comprises a payload moiety, a spacer moiety and a peptide moiety, and the spacer moiety is present between the peptide moiety and the payload moiety. In one embodiment, the linker-payload moiety comprises a payload moiety, a spacer moiety and a peptide moiety, and the spacer moiety is present between the peptide moiety and the payload moiety.

In one embodiment, the spacer moiety has the structure:

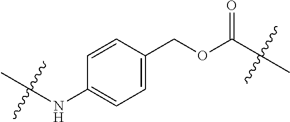 [SMILES: *Nc1ccc(COC(*)=O)cc1]

In some embodiments, the peptide linker comprises, consists essentially of, or consists of a Val-Cit moiety, the spacer moiety has the structure:

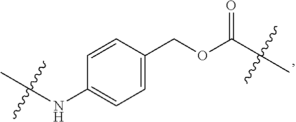 [SMILES: *Nc1ccc(COC(*)=O)cc1]

the —NH— moiety thereof being conjugated to the C-terminus of the Cit residue of the peptide moiety.

In some embodiments, the peptide moiety comprises, consists essentially of, or consists of a Val-Cit moiety, the spacer moiety has the structure:

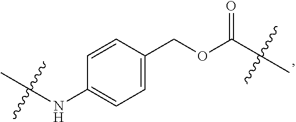 [SMILES: *Nc1ccc(COC(*)=O)cc1]

the —NH— moiety thereof being conjugated to the C-terminus of the Cit residue of the peptide moiety and the C(═O)— moiety thereof being conjugated to the payload moiety.

In some embodiments, the peptide moiety comprises, consists essentially of, or consists of a Val-Cit moiety, the spacer moiety has the structure:

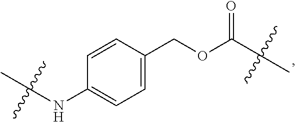 [SMILES: *Nc1ccc(COC(*)=O)cc1]

the —NH— moiety thereof being conjugated to the C-terminus of the Cit residue of the peptide moiety and the C(═O)— moiety thereof being conjugated to an N atom on the payload moiety.

In one embodiment, the payload is a cytotoxic drug. In one embodiment, the payload is an immunomodulator. In one embodiment, the cytotoxic drug is a tubulin polymerisation inhibitor, a DNA damaging agent, a TOPO1 inhibitor, an auristatin, a maytansinoid, a maytansine, or a calicheamicin. In one embodiment, the cytotoxic drug is a TOPO1 inhibitor. In one embodiment, the TOPO1 inhibitor is a camptothecin or an exatecan, or a derivative thereof.

In one embodiment, the payload is a topoisomerase inhibitor, preferably a topoisomerase 1 (TOPO1) inhibitor.

In one embodiment, the TOPO1 inhibitor is deruxtecan.

In one embodiment, the payload is an auristatin.

In one embodiment the binding agent (e.g. bispecific antibody) and/or the antibody drug conjugate is covalently bound to a diagnostic marker, e.g. selected from a radionuclide suitable for radio imaging. For diagnostic purposes, the payload of the antibody drug conjugate as defined herein is a radionuclide suitable for radio imaging.

### Linker-Payload Structures Containing a 6-Ring Exatecan Moiety Linked Via an Open-Chain Group

In one embodiment, the payload comprises a structure shown as formula (V-A):

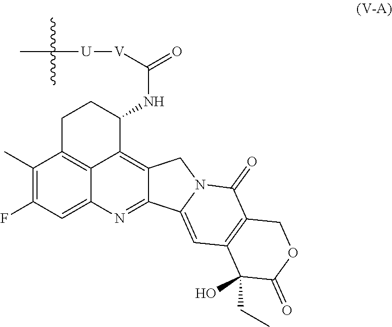

or a tautomer, mesomer, enantiomer, a diastereoisomer thereof, or a pharmaceutically acceptable salt thereof wherein,
U is O or S; and
V is (C1-6) alkylene, (C3-10) cycloalkylene, or (C1-6) alkylene-(C3-10) cycloalkylene.

In one embodiment, U is O. In one embodiment, U is S.

In one embodiment, V is (C1-6) alkylene. In one embodiment, V is (C1-4) alkylene. In one embodiment, V is (C1-3) alkylene. In one embodiment, V is methylene. In one embodiment, V is ethylene. In one embodiment, V is methylmethylene. In one embodiment, V is trimethylene. In one embodiment, V is (S)-methylethylene. In one embodiment, V is (R)-methylethylene.

In one embodiment, V is (C3-10) cycloalkylene. In one embodiment, V is (C3-6) cycloalkylene. In one embodiment, V is (C3-5) cycloalkylene. In one embodiment, V is cyclopropylene. In one embodiment, V is 1,1-cyclopropylene. In one embodiment, V is cis-1,2-cyclopropylene. In one embodiment, V is trans-1,2-cyclopropylene. In one embodiment, V is cyclobutylene. In one embodiment, V is 1,1-cyclobutylene. In one embodiment, V is cis-1,2-cyclobutylene. In one embodiment, V is trans-1,2-cyclobutylene. In one embodiment, V is cis-1,3-cyclobutylene. In one embodiment, V is trans-1,3-cyclobutylene.

In one embodiment, the linker-payload moiety comprises a structure shown as formula (V-C):

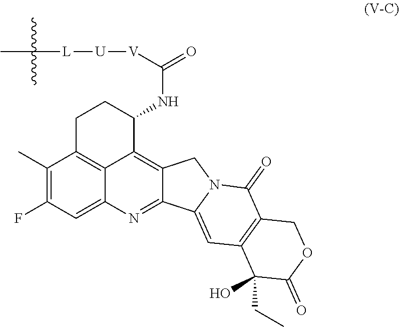

or a tautomer, mesomer, enantiomer a diastereoisomer thereof, or a pharmaceutically acceptable salt thereof wherein,
L is -La-Lb-Lc-;
-La- is selected from the group consisting of

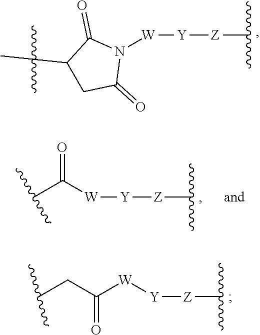

wherein W is —(C(Rwa)(Rwb))wn-, Y is —(OCH2CH2)yn-Oyp, and Z is —(C(Rza)(Rzb))zn; wherein wn is selected from the group consisting of integers ≥0, and 0 or at least 1 methylene unit of W is independently replaced by -Cyr-, —N(Rwx)C(O)—, —C(O)N(Rwx)-, —C(O)—, —OC(O)—, —C(O)O—, —NRwx-, —O—, —S—, —SO—, —SO2-, —P(Rwx)-, —P(═O)(Rwx)-, —N(Rwx)SO2-, —SO2N(Rwx)-, —C(═S)—, —C(═NRwx)-, —N═N—, —C═N—, —N═C— or —C(═N2)-;
wherein yn is selected from the group consisting of integers ≥0, and yp is 0 or 1;
wherein zn is selected from the group consisting of integers ≥0, and
0 or at least 1 methylene unit of Z is independently replaced by -Cyr-, —N(Rzx)C(O)—, —C(O)N(Rzx)-, —C(O)—, —OC(O)—, —C(O)O—, —NRzx-, —O—, —S—, —SO—, —SO2-, —P(Rzx)-, —P(═O)(Rzx)-, —N(Rzx)SO2-, —SO2N(Rzx)-, —C(═S)—, —C(═NRzx)-, —N═N—, —C═N—, —N═C— or —C(═N2)-;
-Cyr- is selected from the group consisting of 6-10 membered arylene, 5-8 membered heteroarylene, 3-10 membered heterocyclylene and 3-10 membered saturated or partially unsaturated carbocyclylene, wherein -Cyr- is unsubstituted or independently substituted with at least 1 substituent Rcx;
wherein each Rwa, each Rwb, each Rza, each Rzb, each Rwx, each Rzx and each Rcx are each independently hydrogen, protium, deuterium, tritium, halogen, —NO2, —CN, —ORr, —SRr, —N(Rra)(Rrb), —C(O)Rr, —CO2Rr, —C(O)C(O)Rr, —C(O)CH2C(O)Rr, —S(O)Rr, —S(O)2Rr, —C(O)N(Rra)(Rrb), —SO2N(Rra)(R^{rb}), —OC(O)Rr, —N(R)SO2Rr, or a C1-6 aliphatic group optionally substituted with Rr;
wherein each Rr, each Rra and each R^{rb} are each independently hydrogen, protium, deuterium, tritium, halogen, —NO2, —CN, —OH, —SH, —NH2, —C(O)H, —CO2H, —C(O)C(O)H, —C(O)CH2C(O)H, —S(O)H, —S(O)2H, —C(O)NH2, —SO2NH2, —OC(O)H, —N(H)SO2H or a C1-6 aliphatic group;
-Lb- represents a peptide residue consisting of 2 to 7 amino acids;
-Lc- is selected from the group consisting of

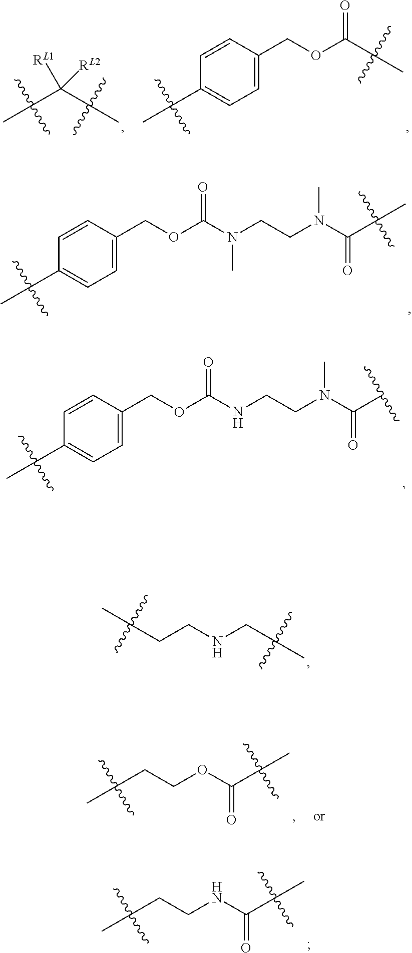

wherein RL1 and RL2 are each independently selected from the group consisting of hydrogen, protium, deuterium, tritium, halogen, —NO2, —CN, —OH, —SH, —NH2, —C(O)H, —CO2H, —C(O)C(O)H, —C(O)CH2C(O)H, —S(O)H, —S(O)2H, —C(O)NH2, —SO2NH2, —OC(O)H, —N(H)SO2H and a C1-6 aliphatic group;
U is O or S; and
V is (C1-6) alkylene, (C3-10) cycloalkylene, or (C1-6) alkylene-(C3-10) cycloalkylene.

In one embodiment, La- is

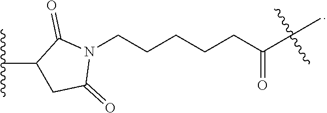 [SMILES: *C(=O)CCCCCN1C(=O)CC(*)C1=O]

In one embodiment, -Lb- is:

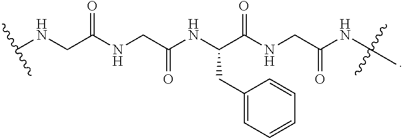 [SMILES: *NCC(=O)NCC(=O)N[C@@H](Cc1ccccc1)C(=O)NCC(=O)N*]

In one embodiment, -Lc- is

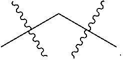 [SMILES: *C*]

In one embodiment, -La-Lb-Lc- is

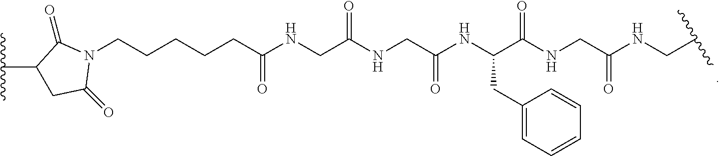 [SMILES: *CNC(=O)CNC(=O)[C@H](Cc1ccccc1)NC(=O)CNC(=O)CNC(=O)CCCCCN1C(=O)CC(*)C1=O]

In one embodiment, U is O. In one embodiment, U is S.

In one embodiment, V is (C1-6) alkylene. In one embodiment, V is (C1-4) alkylene. In one embodiment, V is (C1-3) alkylene. In one embodiment, V is methylene. In one embodiment, V is ethylene. In one embodiment, V is methylmethylene. In one embodiment, V is trimethylene. In one embodiment, V is (S)-methylethylene. In one embodiment, V is (R)-methylethylene.

In one embodiment, V is (C3-10) cycloalkylene. In one embodiment, V is (C3-6) cycloalkylene. In one embodiment, V is (C3-5) cycloalkylene. In one embodiment, V is cyclopropylene. In one embodiment, V is 1,1-cyclopropylene. In one embodiment, V is cis-1,2-cyclopropylene. In one embodiment, V is trans-1,2-cyclopropylene. In one embodiment, V is cyclobutylene. In one embodiment, V is 1,1-cyclobutylene. In one embodiment, V is cis-1,2-cyclobutylene. In one embodiment, V is trans-1,2-cyclobutylene. In one embodiment, V is cis-1,3-cyclobutylene. In one embodiment, V is trans-1,3-cyclobutylene.

In one embodiment, the payload comprises a structure shown as formula (III-A):

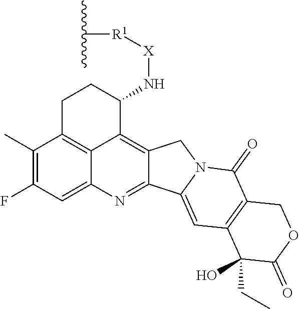

or a tautomer, mesomer, enantiomer, a diastereoisomer thereof, or a pharmaceutically acceptable salt thereof wherein,
R¹ is selected from the group consisting of: —O—, —(R²)N—, —P(═O)(R²)— and —S—;
X is -L¹-CH₂—C(O)—;
L¹ is —(C(R^{3a})(R^{3b}))ₘ—, wherein 0 or at least 1 methylene unit of L¹ is independently replaced by —C(O)—, —C(═S)—, —C(═NR^{4b})— or —C(═N₂)—;
wherein each R², each R^{3a}, each R^{3b} and each R^{4b} are each independently hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group optionally substituted with R;
wherein each R, each R^{a} and each R^{b} are each independently hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C1-6 aliphatic group;
m is selected from the group consisting of integers ≥0,
when R1 is —O— or —HN—, at least 1 methylene unit of L1 is independently replaced by —C(O)—, —C(═S)—, —C(═NR4b)- or —C(═N2)-, or each R3a and each R3b are not both hydrogen.

In one embodiment, the linker-payload moiety comprises a structure shown as formula (III-C):

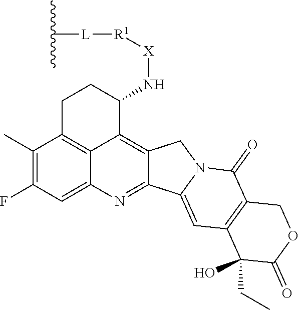

or a tautomer, mesomer, enantiomer a diastereoisomer thereof, or a pharmaceutically acceptable salt thereof wherein,
L is -La-Lb-Lc-;
-Lₐ- is selected from the group consisting of

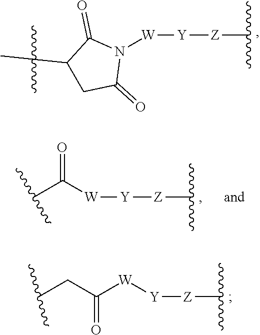

wherein W is —(C(Rwa)(Rwb))wn-, Y is —(OCH2CH2)yn-Oyp, and Z is —(C(Rza)(Rzb))zn;
wherein wn is selected from the group consisting of integers ≥0, and
0 or at least 1 methylene unit of W is independently replaced by -Cyr-, —N(Rwx)C(O)—, —C(O)N(Rwx)-, —C(O)—, —OC(O)—, —C(O)O—, —NRwx-, —O—, —S—, —SO—, —SO2-, —P(Rwx)-, —P(═O)(Rwx)-, —N(Rwx)SO2-, —SO2N(Rwx)-, —C(═S)—, —C(═NRwx)-, —N═N—, —C═N—, —N═C— or —C(═N2)-;
wherein yn is selected from the group consisting of integers ≥0, and yp is 0 or 1;
wherein zn is selected from the group consisting of integers ≥0, and
0 or at least 1 methylene unit of Z is independently replaced by -Cyr-, —N(Rzx)C(O)—, —C(O)N(Rzx)-, —C(O)—, —OC(O)—, —C(O)O—, —NRzx-, —O—, —S—, —SO—, —SO2-, —P(Rzx)-, —P(═O)(Rzx)-, —N(Rzx)SO2-, —SO2N(Rzx)-, —C(═S)—, —C(═NRzx)-, —N═N—, —C═N—, —N═C— or —C(═N2)-;
-Cyr- is selected from the group consisting of: 6-10 membered arylene, 5-8 membered heteroarylene, 3-10 membered heterocyclylene and 3-10 membered saturated or partially unsaturated carbocyclylene, wherein -Cyr- is unsubstituted or independently substituted with at least 1 substituent Rcx;
wherein each Rwa, each Rwb, each Rza, each Rzb, each Rwx, each Rzx and each Rcx are each independently hydrogen, protium, deuterium, tritium, halogen, —NO2, —CN, —ORr, —SRr, —N(Rra)(R^{rb}), —C(O)Rr, —CO2Rr, —C(O)C(O)Rr, —C(O)CH2C(O)Rr, —S(O)Rr, —S(O)2Rr, —C(O)N(Rra)(R^{rb}), —SO2N(Rra)(R^{rb}), —OC(O)Rr, —N(R)SO2Rr, or a C1-6 aliphatic group optionally substituted with Rr;
wherein each Rr, each Rra and each Rrb are each independently hydrogen, protium, deuterium, tritium, halogen, —NO2, —CN, —OH, —SH, —NH2, —C(O)H, —CO2H, —C(O)C(O)H, —C(O)CH2C(O)H, —S(O)H, —S(O)2H, —C(O)NH2, —SO2NH2, —OC(O)H, —N(H)SO2H or a C1-6 aliphatic group;
-L_{b}- represents a peptide residue consisting of 2 to 7 amino acids;
-Lc- is selected from the group consisting of

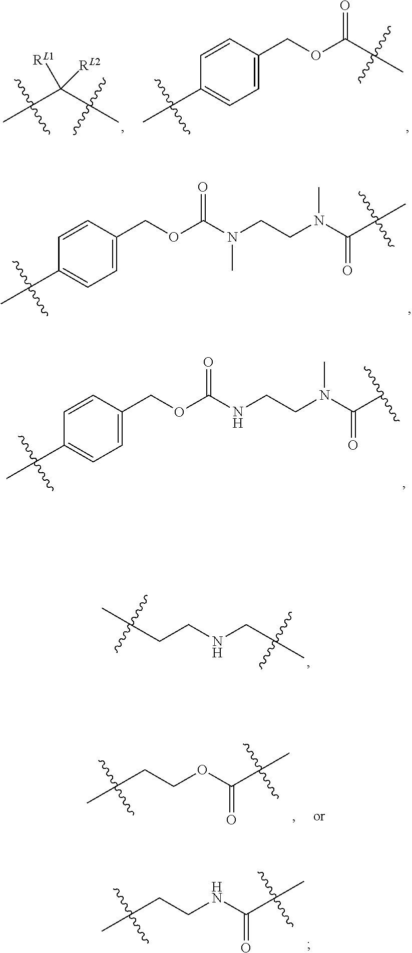

wherein RL1 and RL2 are each independently selected from the group consisting of hydrogen, protium, deuterium, tritium, halogen, —NO2, —CN, —OH, —SH, —NH2, —C(O)H, —CO2H, —C(O)C(O)H, —C(O)CH2C(O)H, —S(O)H, —S(O)2H, —C(O)NH2, —SO2NH2, —OC(O)H, —N(H)SO2H and a C1-6 aliphatic group.

In one embodiment, La- is

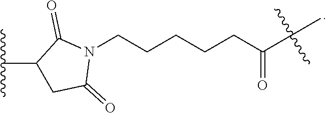 [SMILES: *C(=O)CCCCCN1C(=O)CC(*)C1=O]

In one embodiment, -Lb- is:

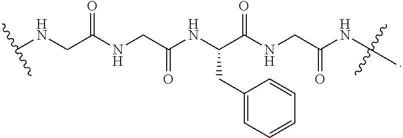 [SMILES: *NCC(=O)NCC(=O)N[C@@H](Cc1ccccc1)C(=O)NCC(=O)N*]

In one embodiment, -Lc- is

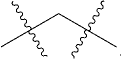 [SMILES: *C*]

In one embodiment -La-Lb-Lc- is

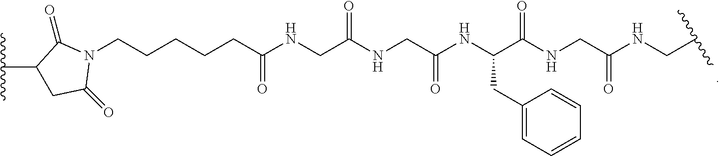 [SMILES: *CNC(=O)CNC(=O)[C@H](Cc1ccccc1)NC(=O)CNC(=O)CNC(=O)CCCCCN1C(=O)CC(*)C1=O]

In one embodiment, the payload has a structure selected from the group consisting of:

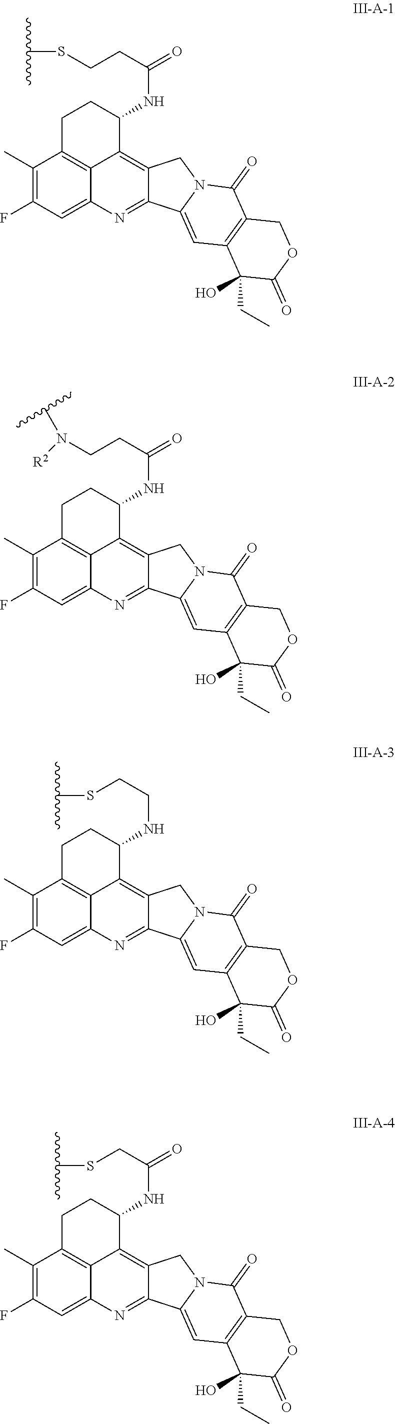

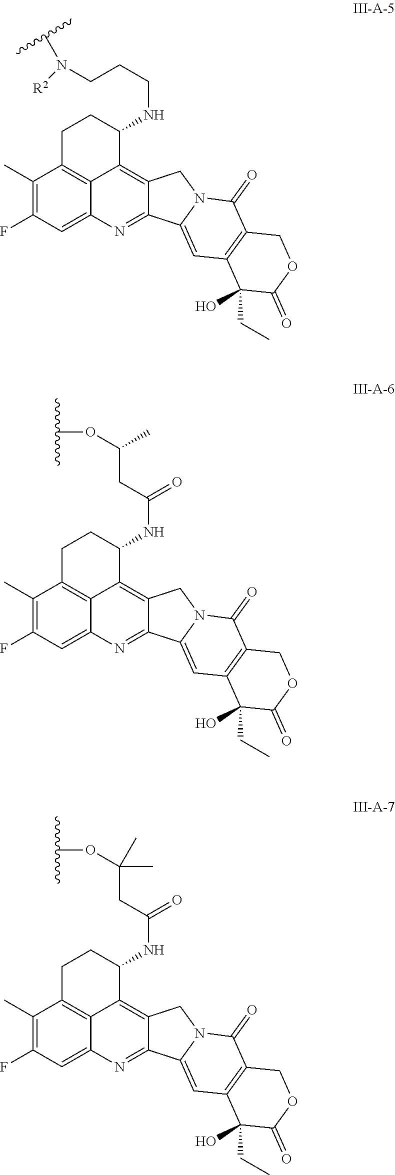

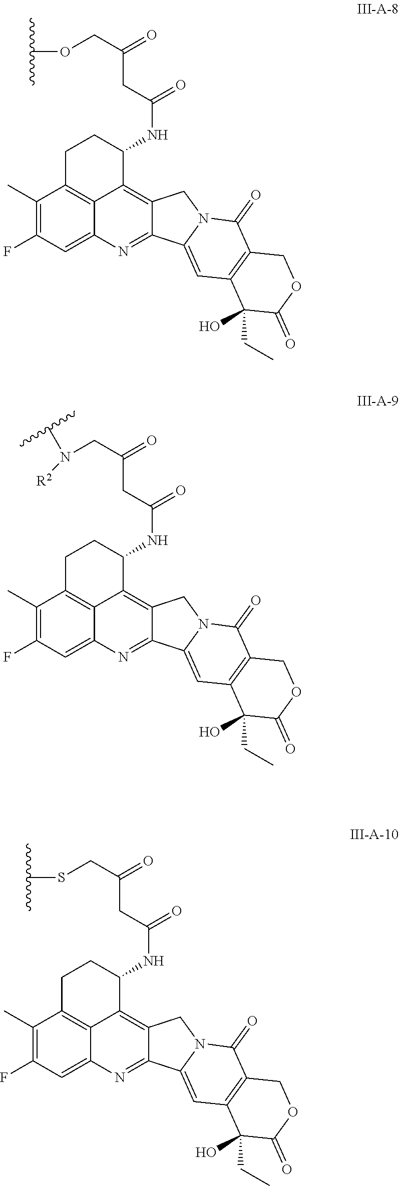

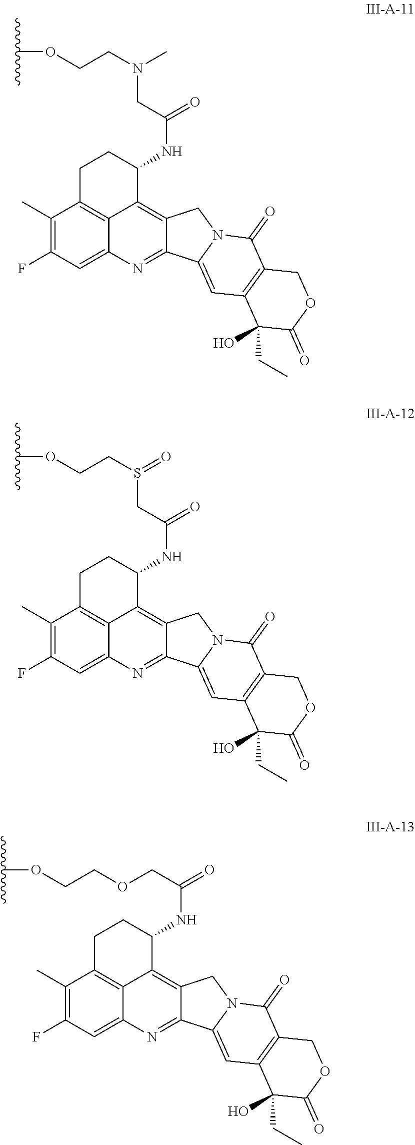 [SMILES: *OCCN(C)CC(=O)N[C@H]1CCc2c(C)c(F)cc3nc4c(c1c23)Cn1c-4cc2c(c1=O)COC(=O)[C@]2(O)CC ; *OCCS(=O)CC(=O)N[C@H]1CCc2c(C)c(F)cc3nc4c(c1c23)Cn1c-4cc2c(c1=O)COC(=O)[C@]2(O)CC ; *OCCOCC(=O)N[C@H]1CCc2c(C)c(F)cc3nc4c(c1c23)Cn1c-4cc2c(c1=O)COC(=O)[C@]2(O)CC]

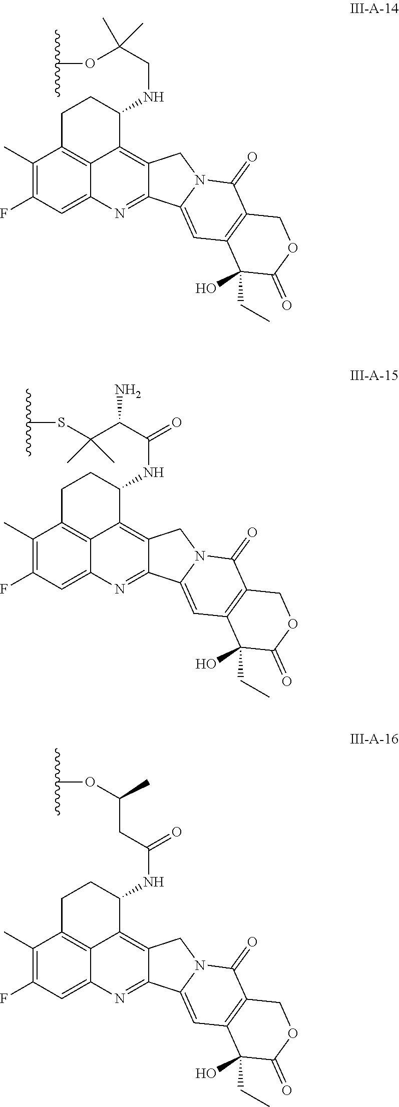 [SMILES: *OC(C)(C)CN[C@H]1CCc2c(C)c(F)cc3nc4c(c1c23)Cn1c-4cc2c(c1=O)COC(=O)[C@]2(O)CC ; *SC(C)(C)[C@H](N)C(=O)N[C@H]1CCc2c(C)c(F)cc3nc4c(c1c23)Cn1c-4cc2c(c1=O)COC(=O)[C@]2(O)CC ; *O[C@@H](C)CC(=O)N[C@H]1CCc2c(C)c(F)cc3nc4c(c1c23)Cn1c-4cc2c(c1=O)COC(=O)[C@]2(O)CC]

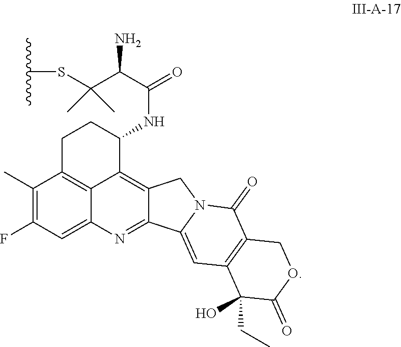 [SMILES: *SC(C)(C)[C@@H](N)C(=O)N[C@H]1CCc2c(C)c(F)cc3nc4c(c1c23)Cn1c-4cc2c(c1=O)COC(=O)[C@]2(O)CC]

In one embodiment, the payload has the following structure:

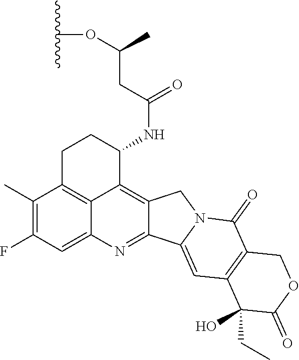 [SMILES: *O[C@@H](C)CC(=O)N[C@H]1CCc2c(C)c(F)cc3nc4c(c1c23)Cn1c-4cc2c(c1=O)COC(=O)[C@]2(O)CC]

In one embodiment, the linker-payload moiety has the following structure:

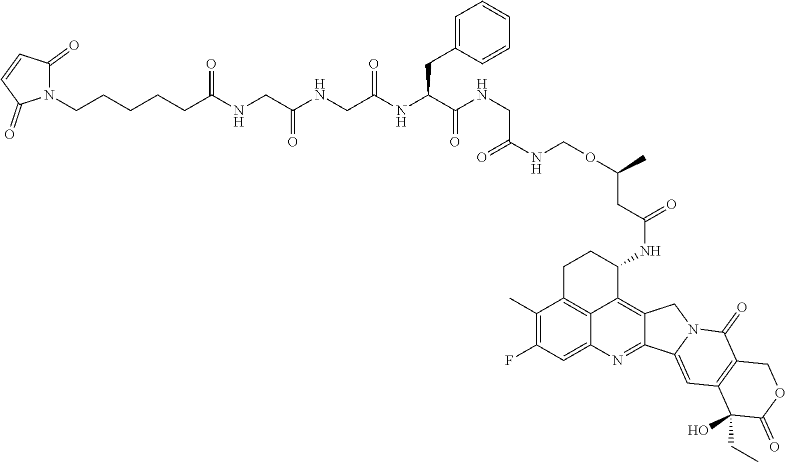 [SMILES: CC[C@@]1(O)C(=O)OCc2c1cc1n(c2=O)Cc2c-1nc1cc(F)c(C)c3c1c2[C@@H](NC(=O)C[C@H](C)OCNC(=O)CNC(=O)[C@H](Cc1ccccc1)NC(=O)CNC(=O)CNC(=O)CCCCCN1C(=O)C=CC1=O)CC3]

Payload and linker-payload moieties having the above structures are described in WO2022/068878 and national applications deriving therefrom.

### Linker Payload Structures Containing a 6-Ring Exatecan Moiety Linked Via a Cyclic Group

In one embodiment, the payload has a structure shown as formula (I-A):

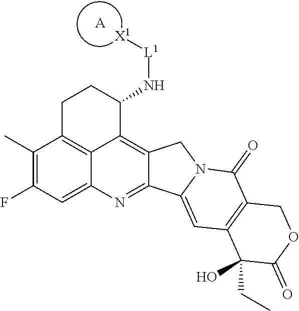

or a tautomer, a mesomer, an enantiomer or a diastereoisomer thereof, or a pharmaceutically acceptable salt thereof, wherein
X¹ is saturated C, and X¹ is substituted with Rⁿ;
ring A is selected from the group consisting of: 3-10 membered saturated or partially unsaturated heterocyclyl and 3-10 membered saturated or partially unsaturated carbocyclyl,
wherein ring A is substituted with 0 or at least 1 substituent R^{1a};
when ring A is 3-10 membered saturated or partially unsaturated carbocyclyl, ring A is substituted with p L², and L² is not Rⁿ;
or, when ring A is 3-10 membered saturated or partially unsaturated heterocyclyl, ring A is substituted with p L²;
L² is —R²-L³-, and R² is used for direct or indirect linking of a ligand;
L³ is —(C(R^{3a})(R^{3b}))ₘ—, wherein when L3 comprises a methylene unit, 0 or at least 1 methylene unit of L³ is independently replaced by —N(R⁴)C(O)—, —C(O)N(R⁴)—, —C(O)—, —OC(O)—, —C(O)O—, —NR⁴—, —O—, —S—, —SO—, —SO2-, —P(R⁴)—, —P(═O)(R⁴)—, —N(R⁴)SO₂—, —SO₂N(R⁴)—, —C(═S)—, —C(═NR⁴)—, —N═N—, —C═N—, —N═C— or —C(═N₂)—;
R² is selected from the group consisting of: —O—, —(R^{2a})N—, —S— and —P(═O)(R^{2a})—;
L¹ is —(C(R^{5a})(R^{5b}))n-, wherein when L¹ comprises a methylene unit, 0 or at least 1 methylene unit of L¹ is independently replaced by —N(R⁶)C(O)—, —C(O)N(R⁶)—, —C(O)—, —OC(O)—, —C(O)O—, —NR⁶—, —O—, —S—, —SO—, —SO2-, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N2)-;
wherein each R^{1a}, each R^{2a}, each R^{3a}, each R^{3b}, each R⁴, each R^{5a}, each R^{5b}, each R⁶ and each ^{Rn} are each independently hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C1-6 aliphatic group optionally substituted with R;
wherein each R, each R^{a} and each R^{b} are each independently hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C1-6 aliphatic group;
m and n are each independently selected from the group consisting of integers ≥0, and p is an integer ≥1.

In one embodiment, the linker-payload has a structure shown as formula (II-Cx):

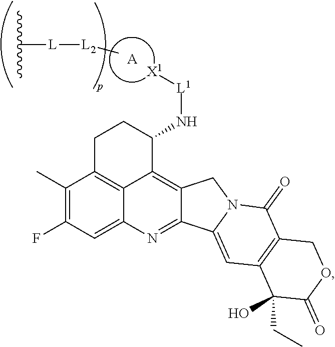

or a tautomer, a mesomer, an enantiomer or a diastereoisomer thereof, or a pharmaceutically acceptable salt thereof wherein, L is -La-Lb-Lc-;
-La- is selected from the group consisting of

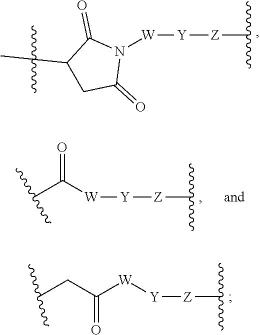

wherein W is —(C(R^{wa})(R^{wb}))_{wn}—, Y is —(OCH₂CH₂)_{yn}—O_{yp}—, and Z is —(C(R^{za})(R^{zb}))_{zn};
wherein wn is selected from the group consisting of integers ≥0, and
0 or at least 1 methylene unit of W is independently replaced by -Cyr-, —N(R^{wx})C(O)—, —C(O)N(R^{wx})—, —C(O)—, —OC(O)—, —C(O)O—, —NR^{wx}—, —O—, —S—, —SO—, —SO₂—, —P(R^{wx})—, —P(═O)(R^{wx})—, —N(R^{wx})SO₂—, —SO₂N(R^{wx})—, —C(═S)—, —C(═NRWX)—, —N═N—, —C═N—, —N═C— or —C(═N₂)—;
wherein yn is selected from the group consisting of integers ≥0, and yp is 0 or 1;
wherein zn is selected from the group consisting of integers ≥0, and
0 or at least 1 methylene unit of Z is independently replaced by -Cyr-, —N(R^{zx})C(O)—, —C(O)N(R^{zx})—, —C(O)—, —OC(O)—, —C(O)O—, —NR—, —O—, —S—, —SO—, —SO₂—, —P(R^{wx})—, —P(═O)(R^{zx})—, —N(R)SO₂—, —SO₂N(R^{zx})—, —C(═S)—, —C(═NRW)—, —N═N—, —C═N—, —N═C— or —C(═N₂)—;
-Cyr- is selected from the group consisting of 6-10 membered arylene, 5-8 membered heteroarylene, 3-10 membered heterocyclylene and 3-10 membered saturated or partially unsaturated carbocyclylene, wherein -Cyr- is unsubstituted or independently substituted with at least 1 substituent R^{cx};
wherein each R^{wa}, each R^{wb}, each R^{za}, each R^{zb}, each R^{wx}, each R^{zx} and each R^{cx} are each independently hydrogen, protium, deuterium, tritium, halogen, —NO₂, —CN, —OR^{r}, —SR^{r}, —N(R^{ra})(R^{rb}), —C(O)R^{r}, —CO₂R^{r}, —C(O)C(O)R^{r}, —C(O)CH₂C(O)R^{r}, —S(O)R^{r}, —S(O)₂R^{r}, —C(O)N(R^{ra})(R^{rb}), —SO₂N(R^{ra})(R^{rb}), —OC(O)R^{r}, —N(R)SO₂R^{r}, or a C₁₋₆ aliphatic group optionally substituted with R^{r};
wherein each R^{r}, each R^{ra} and each R^{rb} are each independently hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
-L_{b}- represents a peptide residue consisting of 2 to 7 amino acids;
-Lc- is selected from the group consisting of

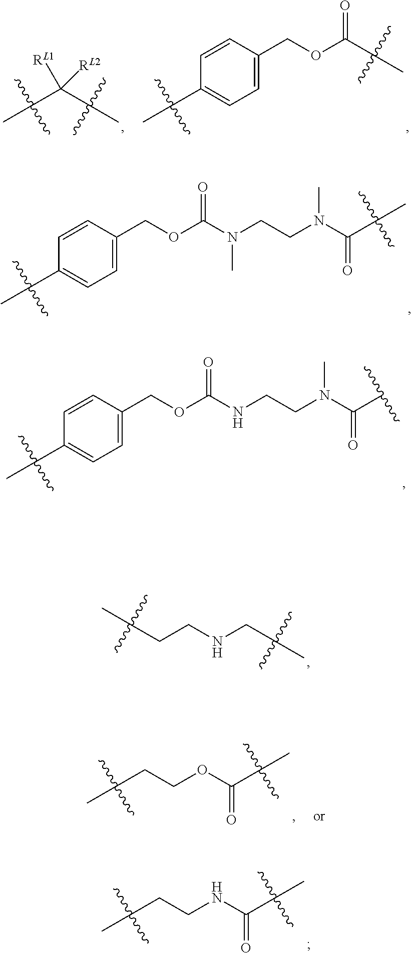

wherein RL1 and RL2 are each independently selected from the group consisting of hydrogen, protium, deuterium, tritium, halogen, —NO2, —CN, —OH, —SH, —NH2, —C(O)H, —CO2H, —C(O)C(O)H, —C(O)CH2C(O)H, —S(O)H, —S(O)2H, —C(O)NH2, —SO2NH2, —OC(O)H, —N(H)SO2H and a C1-6 aliphatic group.

In one embodiment, -La- is selected from the group consisting of:

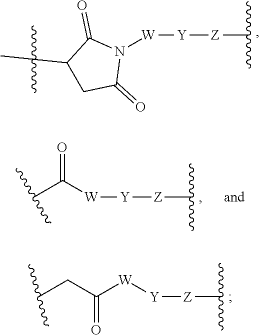

wherein W is —(C(R^{wa})(R^{wb}))_{wn}—, Y is —(OCH₂CH₂)_{yn}—O_{yp}—, and Z is —(C(R^{za})(R^{zb}))_{zn};
wherein wn is selected from the group consisting of integers from 2 to 6, and
0 or 1 methylene unit of W is independently replaced by -Cyr-, —N(R^{wx})C(O)—, —C(O)N(R^{wx})—, —C(O)—, —NR^{wx}— or —O—;
wherein yn is selected from the group consisting of integers from 0 to 12, and yp is 0 or 1;
wherein zn is selected from the group consisting of integers from 0 to 10, and 0 or 1 methylene unit of Z is independently replaced by -Cyr-, —N(Rzx)C(O)—, —C(O)N(Rx)- or —C(O)—;
-Cyr- is selected from the group consisting of 6-10 membered arylene and 3-10 membered saturated or partially unsaturated carbocyclylene, wherein -Cyr- is unsubstituted or independently substituted with 1 to 3 substituent R^{cx};
wherein each R^{wa}, each R^{wb}, each R^{za}, each Rzb, each R^{wx}, each R^{zx} and each R^{cx} are each independently hydrogen, protium, deuterium, tritium, halogen, —NO₂, —CN, —OR^{r}, —SR^{r}, —N(R^{ra})(R^{rb}), —C(O)R^{r}, —CO₂R^{r}, —C(O)C(O)R^{r}, —C(O)CH₂C(O)R^{r}, —S(O)R^{r}, —S(O)₂R^{r}, —C(O)N(R^{ra})(R^{rb}), —SO₂N(R^{ra})(R^{rb}), —OC(O)R^{r}, —N(R)SO₂R^{r}, or a C1-6 aliphatic group optionally substituted with R^{r};
wherein each R^{r}, each R^{ra} and each R^{rb} are each independently hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
-L_{b}- represents a peptide residue consisting of 2 to 7 amino acids, and the peptide residue of -L_{b}- is a peptide residue formed of amino acids selected from the group consisting of: phenylalanine, glycine, alanine, valine, citrulline, lysine, serine, glutamic acid and aspartic acid;
-L_{c}- is selected from the group consisting of:

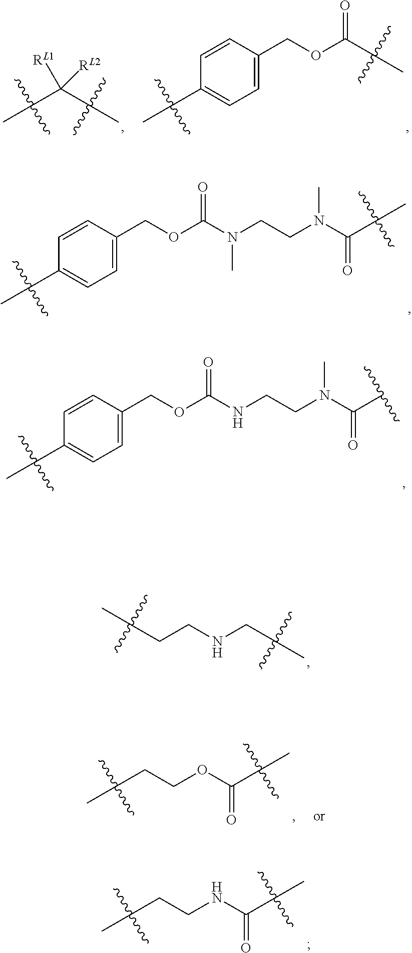

wherein R^{L1} and R^{L2} are each independently selected from the group consisting of hydrogen, protium, deuterium, tritium, halogen, —NO2, —CN, —OH, —SH, —NH2, —C(O)H, —CO2H, —C(O)C(O)H, —C(O)CH2C(O)H, —S(O)H, —S(O)2H, —C(O)NH2, —SO2NH2, —OC(O)H, —N(H)SO2H and a C1-6 aliphatic group.

In one embodiment, -Lₐ- is

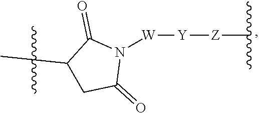

wherein W is —(C(R^{wa})(R^{wb}))_{wn}—, Y is —(OCH₂CH₂)_{yn}—O_{yp}—, and Z is —(C(R^{za})(R^{zb}))_{zn};
wherein wn is 1, 2, 3 or 6, and
1 methylene unit of W is independently replaced by -Cyr-, —N(R^{wx})C(O)—, —C(O)N(R^{wx})— or —C(O)—;
wherein yn is 0, 4 or 8, and yp is 0 or 1;
wherein zn is 1, 2 or 3, and
1 methylene unit of Z is independently replaced by -Cyr-, —N(R^{zx})C(O)—, —C(O)N(R^{zx})— or —C(O)—;
-Cyr- is 3-10 membered saturated carbocyclylene, wherein -Cyr- is unsubstituted or independently substituted with 1 to 3 substituent R^{cx};
wherein each Rwa, each Rwb, each Rza, each Rzb, each Rwx, each Rzx and each Rcx are each independently hydrogen, halogen, —ORr, or a C1-6 aliphatic group optionally substituted with Rr;
wherein each Rr is independently hydrogen, halogen or a C1-6 aliphatic group.

In one embodiment, -La- is

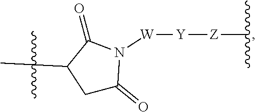

wherein W is —(C(Rwa)(Rwb))wn-, Y is —(OCH2CH2)yn-Oyp-, and Z is —(C(Rza)(Rzb))zn;
wherein wn is 1, 2, 3 or 6, and
1 methylene unit of W is independently replaced by -Cyr-, —N(Rwx)C(O)—, —C(O)N(Rwx)- or —C(O)—;
wherein yn is 0, 4 or 8, and yp is 0 or 1;
wherein zn is 1, 2 or 3, and
1 methylene unit of Z is independently replaced by -Cyr-, —N(Rzx)C(O)—, —C(O)N(Rzx)- or —C(O)—;
-Cyr- is 3-10 membered saturated carbocyclylene, wherein -Cyr- is unsubstituted or independently substituted with 1 to 3 substituent Rcx;
wherein each Rwa, each Rwb, each Rza, each Rzb, each Rwx, each Rzx and each Rcx are each independently hydrogen, halogen, —ORr, or a C1-6 aliphatic group optionally substituted with Rr;
wherein each Rr is independently hydrogen, halogen or a C1-6 aliphatic group.

In one embodiment, -La- is

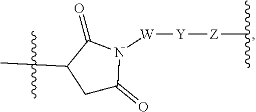

wherein W is —(C(Rwa)(Rwb))wn-, Y is —(OCH2CH2)yn-Oyp-, and Z is —(C(Rza)(Rzb))zn; wherein wn is 1, 2, 3 or 6, and
1 methylene unit of W is independently replaced by -Cyr-, —N(Rwx)C(O)—, —C(O)N(Rwx)- or —C(O)—;
wherein yn is 0, 4 or 8, and yp is 0 or 1;
wherein zn is 1, 2 or 3, and
1 methylene unit of Z is independently replaced by -Cyr-, —N(Rzx)C(O)—, —C(O)N(Rzx)- or —C(O)—;
-Cyr- is 3-10 membered saturated carbocyclylene, wherein -Cyr- is unsubstituted or independently substituted with 1 to 3 substituent Rcx;
wherein each Rwa, each Rwb, each Rza, each Rzb, each Rwx, each Rzx and each Rcx are each independently hydrogen, halogen, —ORr, or a C1-6 aliphatic group optionally substituted with Rr;
wherein each Rr is independently hydrogen, halogen or a C1-6 aliphatic group.

In one embodiment, -La- is

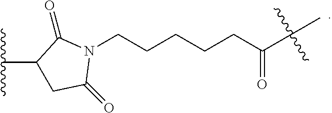 [SMILES: *C(=O)CCCCCN1C(=O)CC(*)C1=O]

In one embodiment, -Lb- represents a peptide residue consisting of 2 to 4 amino acids, and the peptide residue of -Lb- is a peptide residue formed of amino acids selected from the group consisting of phenylalanine, glycine, alanine, valine, citrulline and lysine.

In one embodiment, -Lb- is selected from the group consisting of:

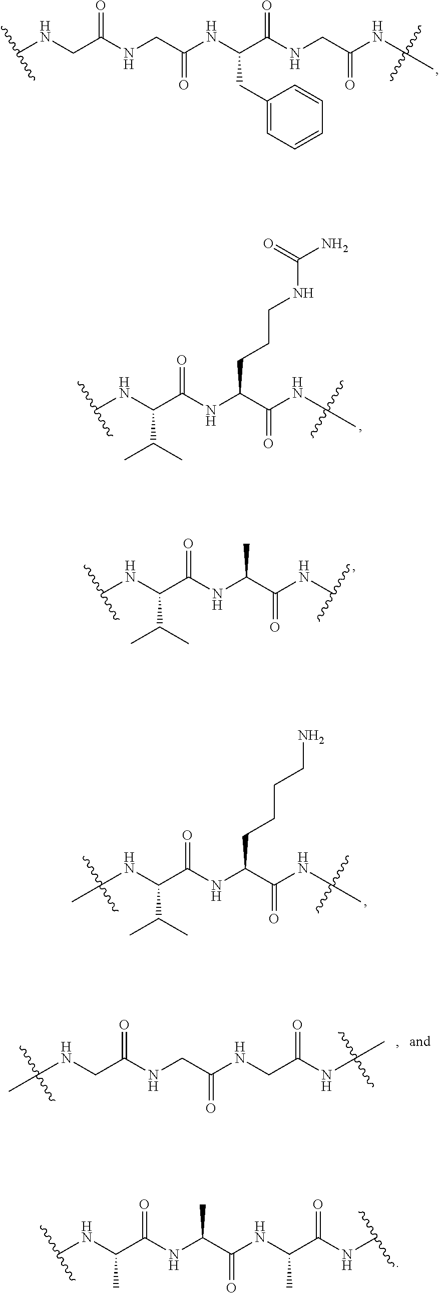 [SMILES: *NCC(=O)NCC(=O)N[C@@H](Cc1ccccc1)C(=O)NCC(=O)N* ; *NC(=O)[C@H](CCCNC(N)=O)NC(=O)[C@@H](N*)C(C)C ; *NC(=O)[C@H](C)NC(=O)[C@@H](N*)C(C)C ; *NC(=O)[C@H](CCCCN)NC(=O)[C@@H](N*)C(C)C ; *NCC(=O)NCC(=O)NCC(=O)N* ; *NC(=O)[C@H](C)NC(=O)[C@H](C)NC(=O)[C@H](C)N*]

In one embodiment, -Lb- is

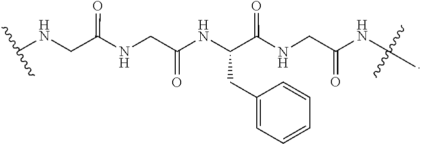 [SMILES: *NCC(=O)NCC(=O)N[C@@H](Cc1ccccc1)C(=O)NCC(=O)N*]

In one embodiment, -Lc- is selected from the group consisting of;

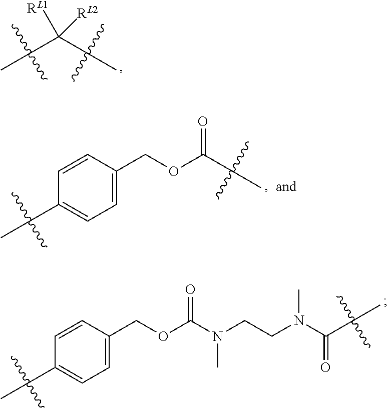

wherein RL1 and RL2 are each independently selected from the group consisting of: hydrogen, halogen, —OH and a C1-6 aliphatic group.

In one embodiment, -Lc- is

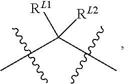

wherein RL1 and RL2 are each independently selected from the group consisting of hydrogen, halogen, —OH and a C1-6 aliphatic group.

In one embodiment, -Lc- is.

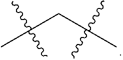 [SMILES: *C*]

In one embodiment, -La-Lb-Lc- is

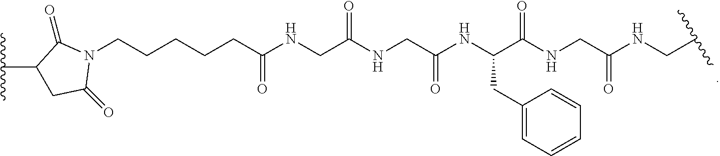 [SMILES: *CNC(=O)CNC(=O)[C@H](Cc1ccccc1)NC(=O)CNC(=O)CNC(=O)CCCCCN1C(=O)CC(*)C1=O]

In one embodiment, ring A is selected from the group consisting of: 3-10 membered saturated heterocyclyl and 3-10 membered saturated carbocyclyl.

In one embodiment, ring A is 3-10 membered saturated carbocyclyl.

In one embodiment, ring A is 3-6 membered saturated carbocyclyl, preferably wherein ring A is 4 membered saturated carbocyclyl. In one embodiment, ring A is 6 membered saturated carbocyclyl.

In one embodiment, ring A is 3-10 membered saturated heterocyclyl. In one embodiment, ring A is 3-6 membered saturated heterocyclyl. In one embodiment, ring A is 3 membered saturated heterocyclyl.

In one embodiment, ring A comprises 1 heteroatom. In one embodiment, ring A comprises 1 nitrogen atom.

In one embodiment, ring A is substituted with 0 substituent R^{1a}In one embodiment, the compound comprises a structure shown as formula (II-Ax):

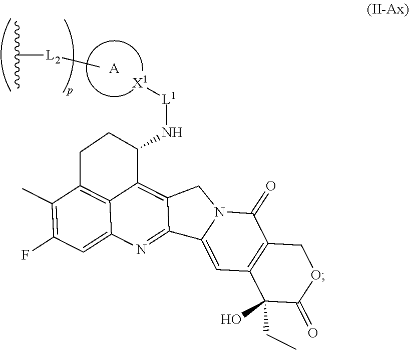

or a tautomer, a mesomer, an enantiomer or a diastereoisomer thereof, or a pharmaceutically acceptable salt thereof wherein, when ring A is 3-10 membered saturated or partially unsaturated carbocyclyl, ring A is substituted with p L², and L² is not Rⁿ;
or, when ring A is 3-10 membered saturated or partially unsaturated heterocyclyl, ring A is substituted with p L².

In one embodiment, ring A is substituted with 1 L².

In one embodiment, m is 0, and L³ is a covalent bond.

In one embodiment, m is 2, and L³ is —(C(R^{3a})(R^{3b}))₂—.

In one embodiment, n is 0 or 1.

In one embodiment, n is 0, and L¹ is a covalent bond.

In one embodiment, n is 1, and L¹ is —C(R^{5a})(R^{5b})—.

In one embodiment, 1 methylene unit of L¹ is replaced by —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—.

In one embodiment, 1 methylene unit of L¹ is replaced by —C(O)—.

In one embodiment, R² is selected from the group consisting of: —O—, —(R^{2a})N- and —S—.

In one embodiment, R² is —O—.

In one embodiment, R² is —(R^{2a})N—.

In one embodiment, R^{2a} is hydrogen.

In one embodiment, R² is —HN—.

In one embodiment, R^{1a} is hydrogen.

In one embodiment, R^{3a} and R^{3b} are each independently hydrogen.

In one embodiment, R⁴ is hydrogen.

In one embodiment, R⁶ is hydrogen.

In one embodiment, R, R^{a} and R^{b} are each independently hydrogen. In one embodiment, the payload comprises the following group of structures:

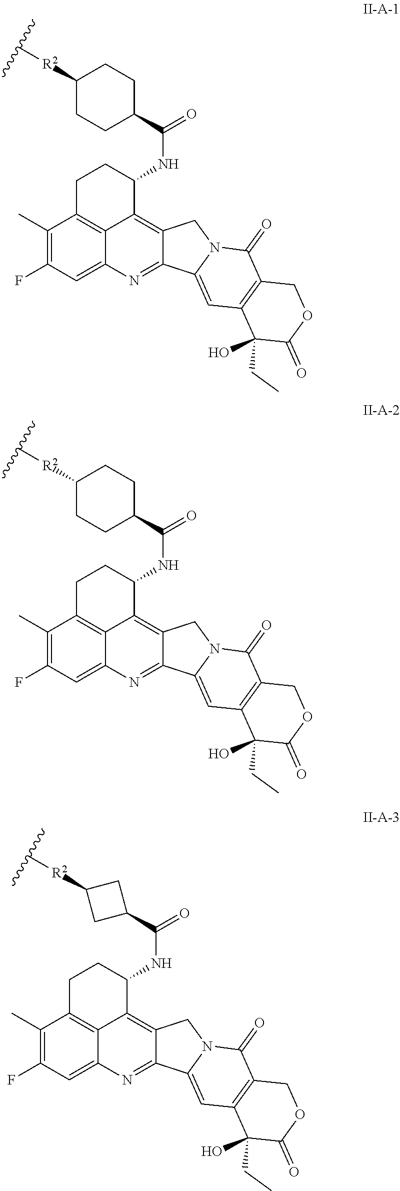

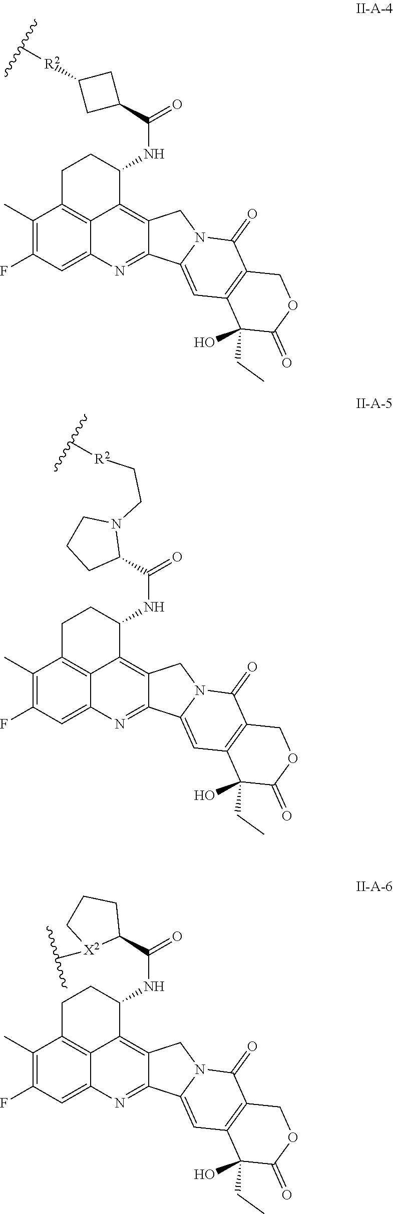

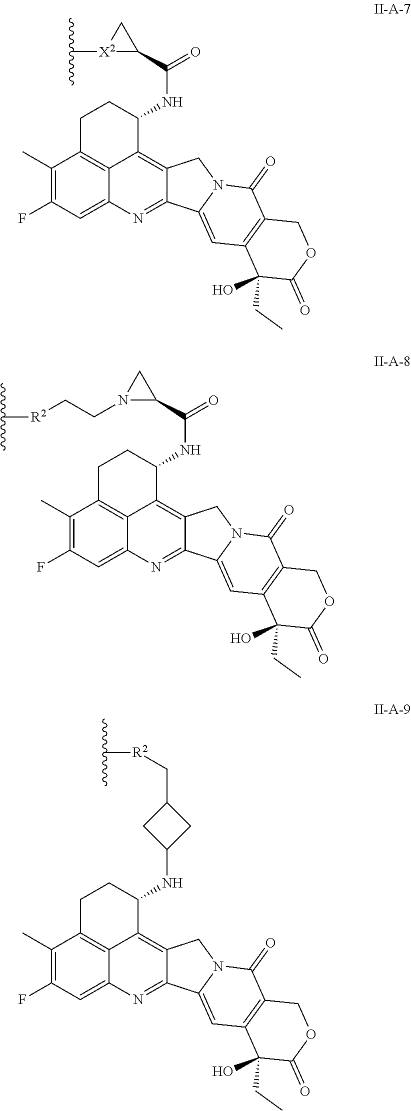

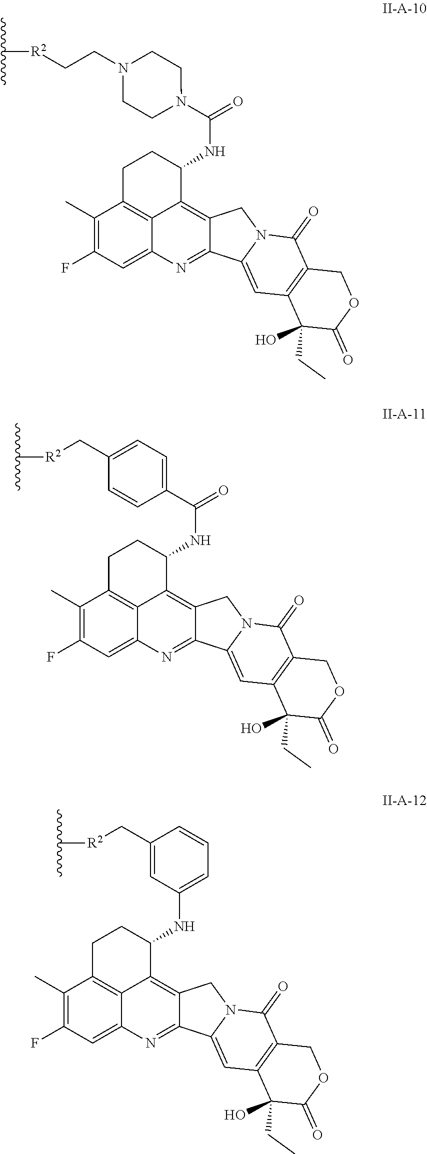

In one embodiment, the payload moiety comprises the structure

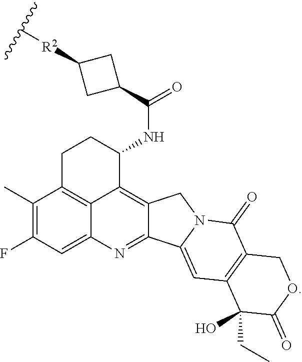

In some embodiments, the payload moiety has the structure

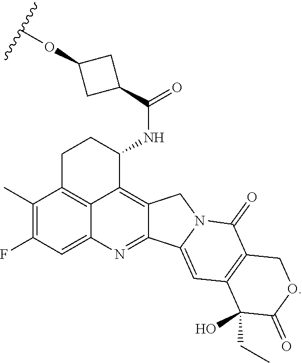 [SMILES: *O[C@H]1C[C@@H](C(=O)N[C@H]2CCc3c(C)c(F)cc4nc5c(c2c34)Cn2c-5cc3c(c2=O)COC(=O)[C@]3(O)CC)C1]

In some embodiments, the linker-payload moiety has the structure

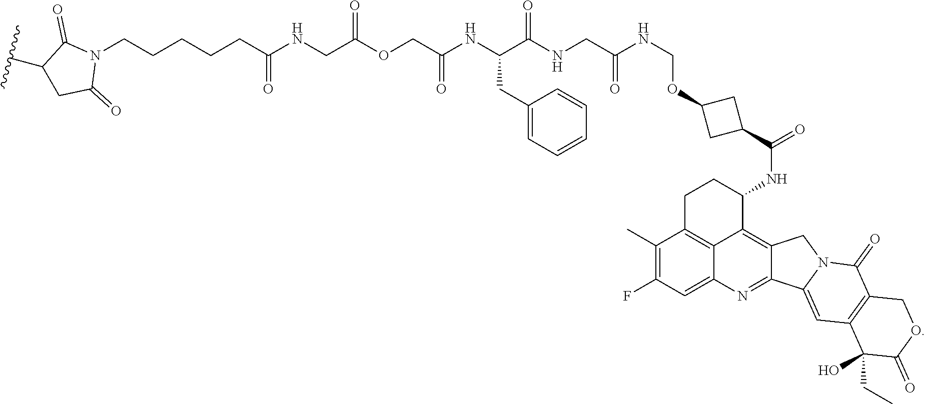 [SMILES: *C1CC(=O)N(CCCCCC(=O)NCC(=O)OCC(=O)N[C@@H](Cc2ccccc2)C(=O)NCC(=O)NCO[C@H]2C[C@@H](C(=O)N[C@H]3CCc4c(C)c(F)cc5nc6c(c3c45)Cn3c-6cc4c(c3=O)COC(=O)[C@]4(O)CC)C2)C1=O]

Payload and linker-payload moieties having the above structures are described in WO2022/068878 and national applications deriving therefrom.

### Linker-Payload Structures Containing a 5- or 6-Ring Camptothecin or Exatecan Moiety Linked Using Alternative Open-Chain or Cyclic Structures

In one embodiment, the linker-payload moiety has the structure represented by formula:

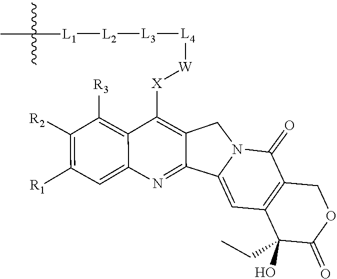

wherein,
L1 is selected from

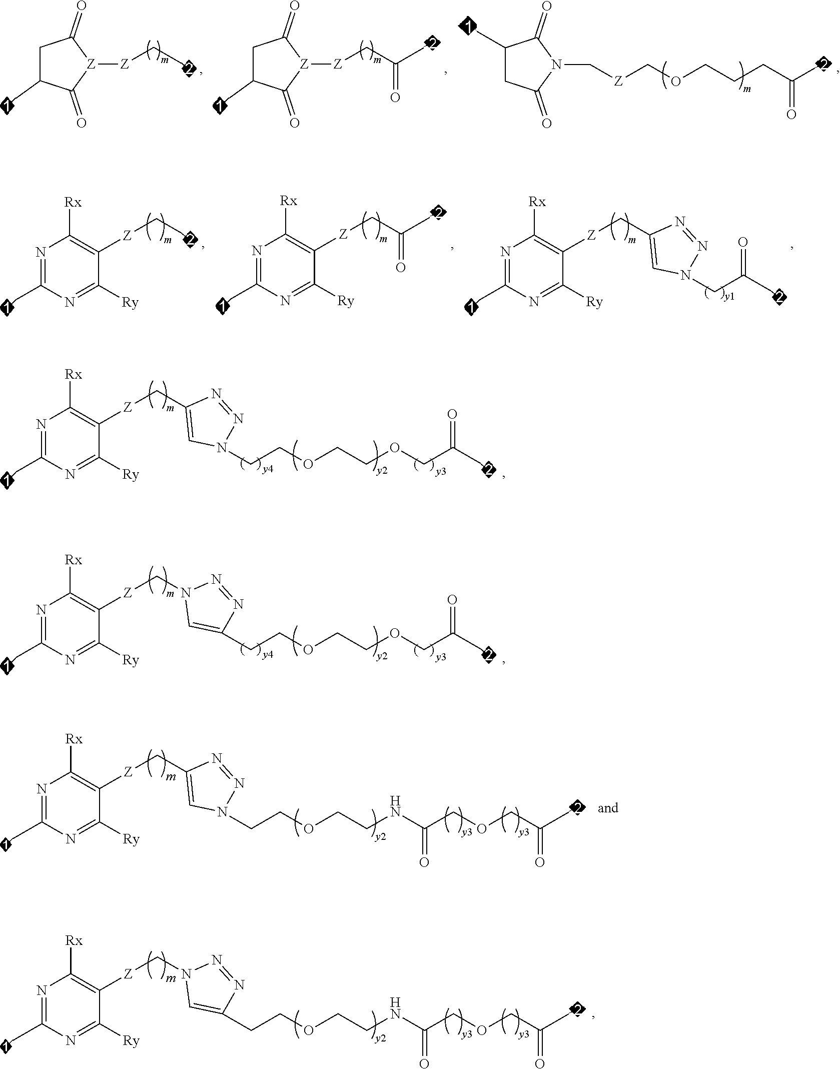

each Z is independently selected from a direct bond, a carbon-carbon triple bond, a carbon-carbon double bond, and amido (preferably selected from a direct bond, a carbon-carbon triple bond, and a carbon-carbon double bond);
Rx and Ry are each independently selected from H and C1-4 alkyl;
each m is independently selected from 0, 1, 2, 3, 4, 5 and 6;
y1 is selected from any integer between 1 and 6 (such as 4, 5, and 6);
each y2 is independently selected from any integer between 0 and 15 (such as 6-15);
each y3 is independently selected from 1, 2, and 3;
each y4 is independently selected from 0 and 1; position 1 is attached to Tb via an S atom, and position 2 is attached to L2 or L3;
L2 is absent or present, and when L2 is present, L2 is selected from:

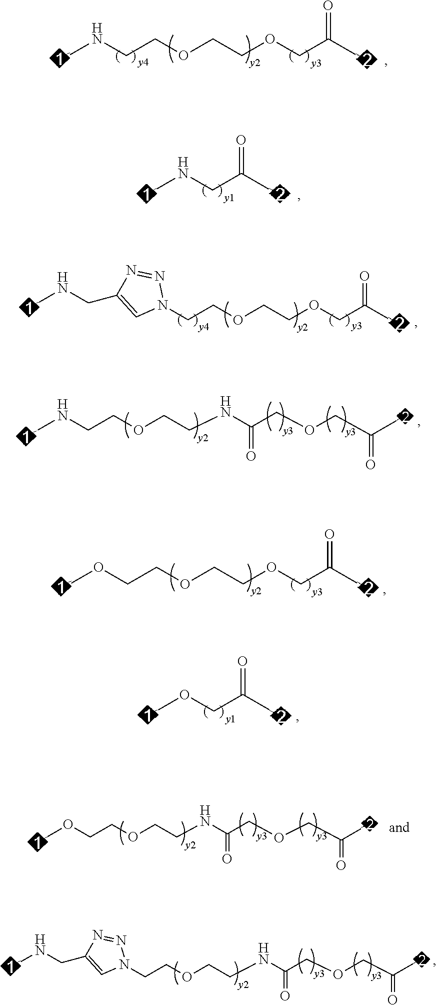

y1 is selected from any integer between 1 and 6 (such as 4, 5, and 6); each y2 is independently selected from any integer between 0 and 10 (such as 6-10); each y3 is independently selected from 1 or 2; each y4 is independently selected from 0 and 1, position 1 is attached to L1, and position 2 is attached to L3;
L3 is selected from an amino acid residue or a short peptide consisting of 2-10 amino acid residues; the amino acid residue is selected from natural amino acid residues, non-natural amino acid residues, or selected from amino acid residue represented by AA1 or stereoisomer thereof;

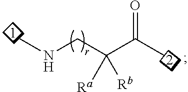

in the amino acid residue represented by AAT, any one of Ra and Rb is H, and the other is H,

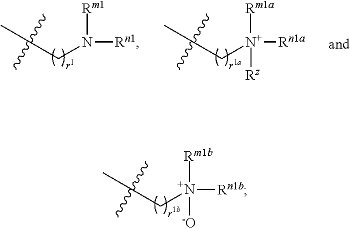

or, R^{a} and R^{b}, together with the carbon atom to which they are both attached, form a 5-6 membered heterocyclic ring, and said 5-6 membered heterocyclic ring is piperidine ring or piperazine ring.
r, r1, r1a and r1b are each independently 0, 1, 2, 3, 4 or 5;
Rm1, Rn1, Rm1a, Rn1a, Rm1b and Rn1b are each independently H, C1-6 alkyl or —COORx1, wherein, Rx1 is C1-6 alkyl;
or, Rm1 and Rn1, Rm1a and Rn1a, and Rm1b and Rn1b, together with the nitrogen atom to which they are both attached, form a 5-6 membered heterocyclic ring, and of said 5-6 membered heterocyclic ring, the heteroatom is selected from 1 or 2 N atoms; the said 5-6 membered heterocyclic ring is optionally substituted with one or more R0′;
Rz is selected from C1-6 alkyl;
R0 and R0′ are each independently selected from C1-6 alkyl, —NRm2Rn2 or 5-6 membered heterocyclyl optionally substituted with C1-6 alkyl; of said 5-6 membered heterocyclyl, the heteroatom is selected from 1 or 2 N atoms;
Rm2 and Rn2 are each independently selected from H and C1-6 alkyl;
L4 is absent or present, when L4 is present, L4 is selected from

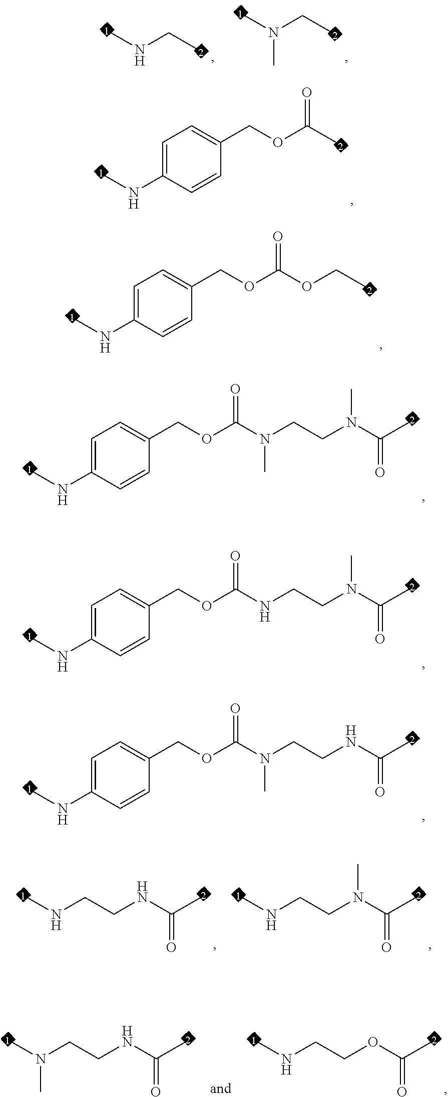

position 1 is attached to L3, and position 2 is attached to D;
R1 and R2 are each independently selected from H, halogens and C1-4 alkyl; or, R1 and R2, together with the carbon atom to which they are both attached form a 5-6 membered heterocyclic ring, the heterocyclic ring contains 1, 2, or 3 of 0, S, or N or any combination thereof,
R3 is selected from H and C1-4 alkyl; or R3 and X, together with the carbon atom to which they are both attached, form a 5-6 membered carbon ring;
W is absent or present, when W is present, W is selected from —O—, —S—, —NR4-,

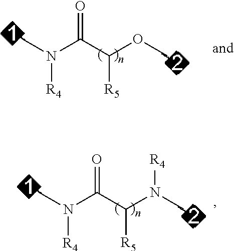

position 1 is attached to X, and position 2 is attached to L4 or L3;
X is selected from optionally substituted —(CH2)n1-,

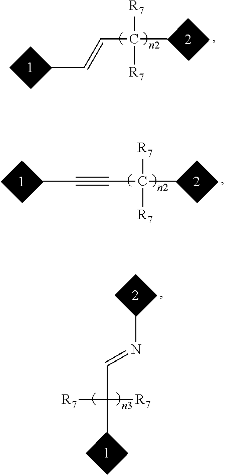

position 1 is attached to the parent ring and position 2 is attached to W or L4; the substituent is selected from one or two C1-4 alkyls;
R4, R5, and R7 are each independently selected from H and C1-4 alkyl;
n, n1, n2, n3 are each independently selected from any integer between 0 and 6.

In one embodiment, L1 is selected from

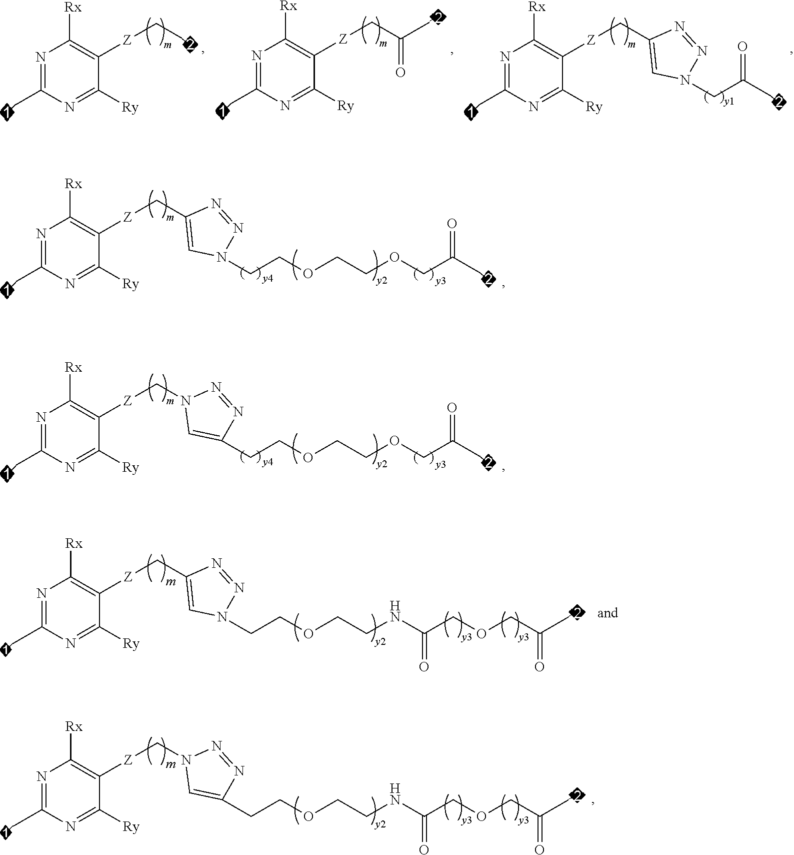

In one embodiment, L1 is

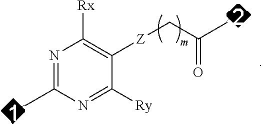

In one embodiment, Z is selected from a direct bond, a carbon-carbon triple bond, and a carbon-carbon double bond.

In one embodiment, Z is a carbon-carbon triple bond.

In one embodiment, Rx is H or methyl.

In one embodiment, Rx is H.

In one embodiment, Ry is H or methyl.

In one embodiment, Ry is H.

In one embodiment, m is 2, 3 or 4. In one embodiment, m is 3.

In one embodiment, L2 is absent.

In one embodiment, L3 is selected from AA1, AA1-Gly, Val-Cit, Val-Ala, Val-AA1, Val-AA1-Gly, AA1-Ala-Asn, Ala-Ala-Ala, Ala-Ala-Asn and Gly-Gly-Phe-Gly.

In one embodiment, L3 is selected from AA1, AA1-Gly, Val-Cit, Val-AA1-Gly, AA1-Ala-Asn and Gly-Gly-Phe-Gly.

In one embodiment, L3 is Val-AA1-Gly.

In one embodiment, the amino acid residue represented by AA1 is selected from

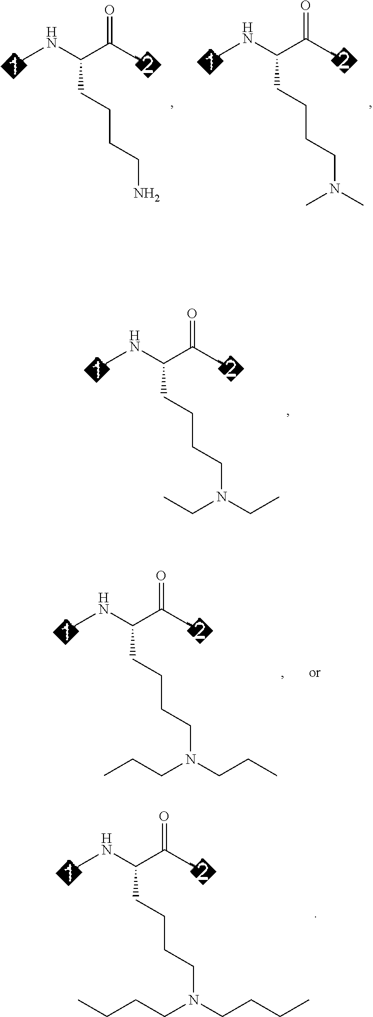

In one embodiment, the amino acid residue represented by AA1 is

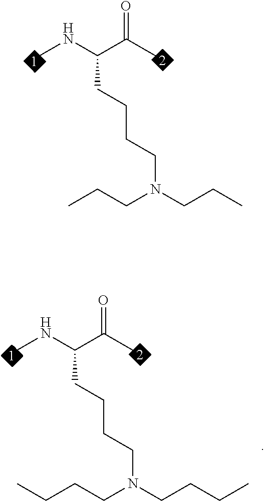

In one embodiment, L4 is selected from

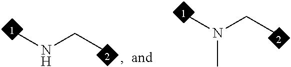

In one embodiment, L4 is

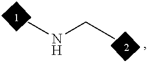

In one embodiment, W is O.

In one embodiment, X is —(CH2)n1-.

In one embodiment, n1 is 2, 3 or 4. In one embodiment, n1 is 3.

In one embodiment, the structure

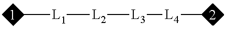

is:

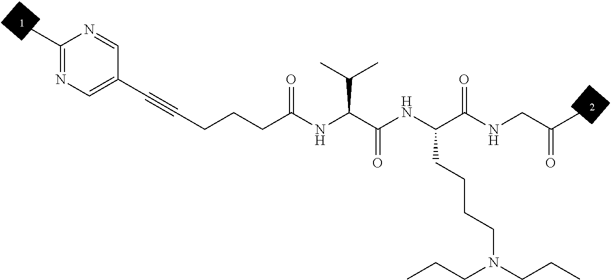

wherein position 1 is attached to the connecting atom on the binding molecule and position 2 is attached to W.

In one embodiment, the structural fragment represented by

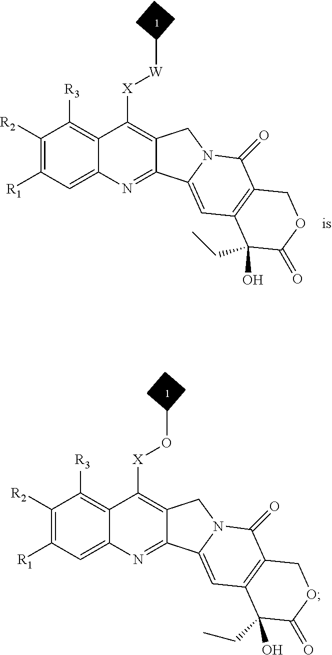

and position 1 is attached to L4.

In one embodiment, the structural fragment

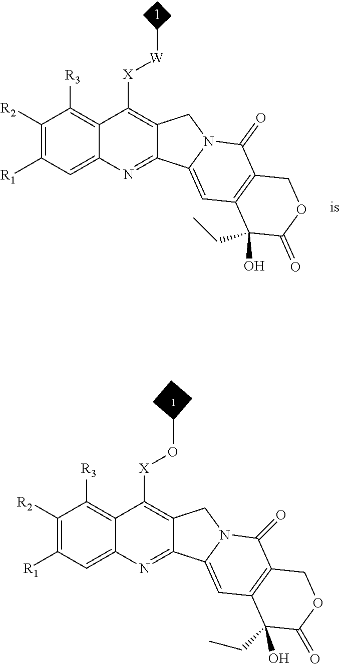

wherein X is —(CH2)3-.

In one embodiment, the linker-payload moiety has the structure:

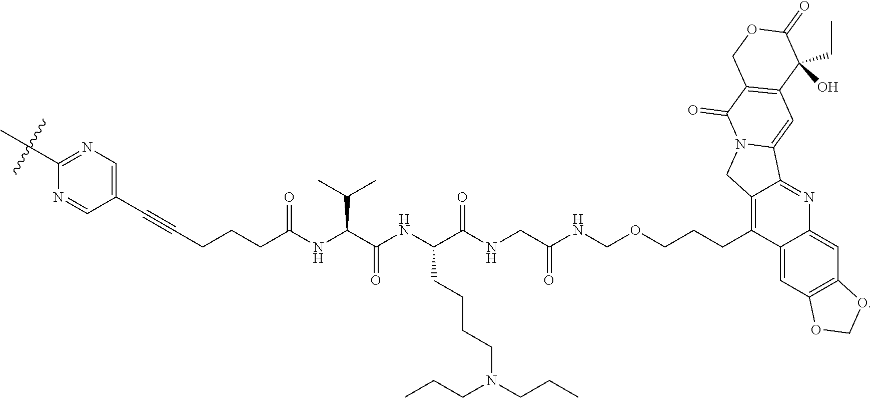 [SMILES: *c1ncc(C#CCCCC(=O)N[C@H](C(=O)N[C@@H](CCCCN(CCC)CCC)C(=O)NCC(=O)NCOCCCc2c3c(nc4cc5c(cc24)OCO5)-c2cc4c(c(=O)n2C3)COC(=O)[C@]4(O)CC)C(C)C)cn1]

In some embodiments, the connection number q is an integer from 4 to 12. In some embodiments, the connection number q is an integer from 6 to 10. In some embodiments, the connection number q is an integer from 7 to 9. In some embodiments, the connection number q is 6. In some embodiments, the connection number q is 7. In some embodiments, the connection number q is 8. In some embodiments, the connection number q is 9. In some embodiments, the connection number q is 10.

Payload and linker-payload moieties having the above structures are described generally in WO2022/170971 and national applications deriving therefrom.

### Linker-Payload Structures Containing Auristatin Moieties

In one embodiment, the payload is monomethyl auristatin E (MMAE). MMAE has the IUPAC name (S)—N-((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide and has the following structure:

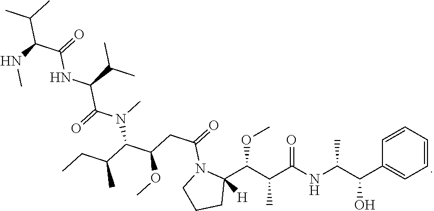 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

In one embodiment, the payload is MMAE and the linker-payload moiety is formed by the conjugation of a linker, as defined herein, to the N-terminal of the MMAE moiety.

In one embodiment, the payload is monomethyl auristatin F (MMAF). MMAF has the IUPAC name (S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)pyrrolidin-2-yl)-3-methoxy-2-methylpropanamido)-3-phenylpropanoic acid and has the following structure:

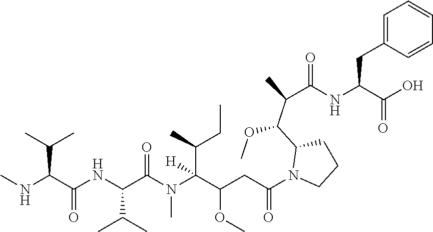 [SMILES: CC[C@H](C)[C@@H](C(CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H](NC)C(C)C)C(C)C]

In one embodiment, the payload is MMAF and the linker-payload moiety is formed by the conjugation of a linker, as defined herein, to the N-terminal of the MMAF moiety. In one embodiment, the payload moiety is MMAE or MMAF and the linker comprises a spacer moiety. In one embodiment, the payload is MMAE or MMAF, the linker comprises a peptide moiety and the spacer moiety is conjugated to the C-terminus of the peptide moiety. In one embodiment, the linker-payload moiety comprises a payload moiety, a spacer moiety and a peptide moiety, the payload moiety is MMAE or MMAF, and the spacer moiety is present between the peptide moiety and the payload moiety. In one embodiment, the linker-payload moiety comprises a payload moiety, a spacer moiety and a peptide moiety, the payload is MMAE or MMAF, and the spacer moiety is present between the peptide moiety and the payload moiety.

In some embodiments, the payload is MMAE or MMAF, the peptide linker comprises, consists essentially of, or consists of a Val-Cit moiety, the spacer moiety has the structure:

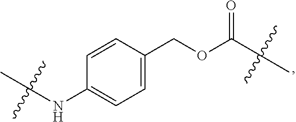 [SMILES: *Nc1ccc(COC(*)=O)cc1]

the —NH— moiety of the spacer moiety being conjugated by an amide bond to the C-terminus of the Cit residue of the peptide moiety and the C(═O)— moiety of the spacer moiety being conjugated by an amide bond to the N-terminus of the MMAE or MMAF payload moiety.

In some embodiments, the payload is MMAE or MMAF, the linker comprises a maleimide moiety, the peptide moiety is a Val-Cit moiety, the spacer moiety has the structure:

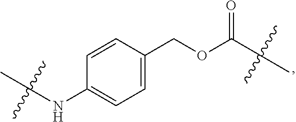 [SMILES: *Nc1ccc(COC(*)=O)cc1]

the —NH— moiety of the spacer moiety being conjugated by an amide bond to the C-terminus of the Cit residue of the peptide moiety and the C(═O)— moiety of the spacer moiety being conjugated by an amide bond to the N-terminus of the MMAE or MMAF payload moiety.

In one embodiment, the linker-payload moiety has the structure of formula (VIIa):

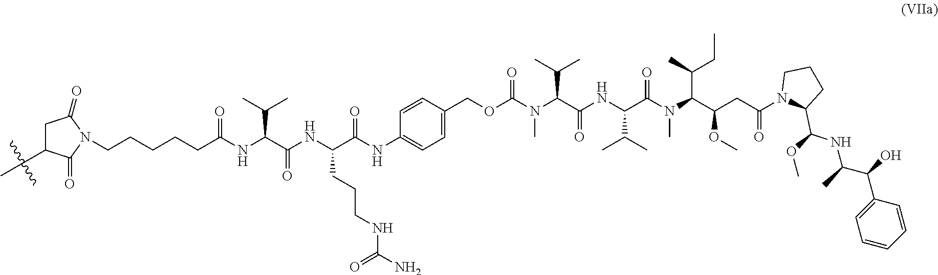 [SMILES: *C1CC(=O)N(CCCCCC(=O)N[C@H](C(=O)N[C@@H](CCCNC(N)=O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@@H](N[C@H](C)[C@@H](O)c3ccccc3)OC)OC)C(C)C)C(C)C)cc2)C(C)C)C1=O]

in which the succinimidyl moiety is formed by conjugation of a suitable group on an amino acid residue (typically a cysteine residue) of the binding molecule with a maleimide group on the linker moiety.

The linker-payload moiety of formula (VII) is also known as vedotin. The preparation of this linker-payload moiety is described in WO2004/010957 and national applications based thereon.

### Antibody-Drug Conjugates

In one embodiment, the antibody-drug conjugate has a structure shown as formula (V-D):

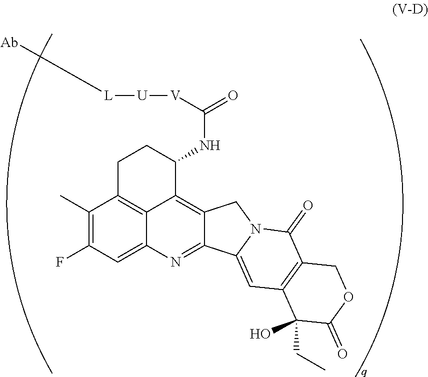

or a tautomer, mesomer, enantiomer or diastereoisomer thereof, or a pharmaceutically acceptable salt thereof wherein:
Ab is an antibody or fragment thereof that specifically binds to a target expressed on the surface of a tumour cell;
the connection number q is an integer from 1 to 16, and
L is -La-Lb-Lc-;
-La- is selected from the group consisting of

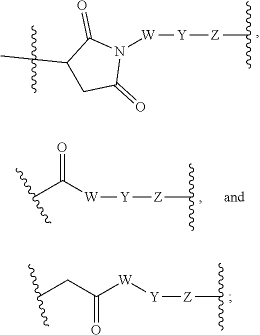

wherein W is —(C(Rwa)(Rwb))wn-, Y is —(OCH2CH2)yn-Oyp, and Z is —(C(Rza)(Rzb))zn;
wherein wn is selected from the group consisting of integers ≥0, and
0 or at least 1 methylene unit of W is independently replaced by -Cyr-, —N(Rwx)C(O)—, —C(O)N(Rwx)-, —C(O)—, —OC(O)—, —C(O)O—, —NRwx-, —O—, —S—, —SO—, —SO2-, —P(Rwx)-, —P(═O)(Rwx)-, —N(Rwx)SO2-, —SO2N(Rwx)-, —C(═S)—, —C(═NRwx)-, —N═N—, —C═N—, —N═C— or —C(═N2)-;
wherein yn is selected from the group consisting of integers ≥0, and yp is 0 or 1;
wherein zn is selected from the group consisting of integers ≥0, and
0 or at least 1 methylene unit of Z is independently replaced by -Cyr-, —N(Rzx)C(O)—, —C(O)N(Rzx)-, —C(O)—, —OC(O)—, —C(O)O—, —NRzx-, —O—, —S—, —SO—, —SO2-, —P(Rzx)-, —P(═O)(Rzx)-, —N(Rzx)SO2-, —SO2N(Rzx)-, —C(═S)—, —C(═NRzx)-, —N═N—, —C═N—, —N═C— or —C(═N2)-;
-Cyr- is selected from the group consisting of 6-10 membered arylene, 5-8 membered heteroarylene, 3-10 membered heterocyclylene and 3-10 membered saturated or partially unsaturated carbocyclylene, wherein -Cyr- is unsubstituted or independently substituted with at least 1 substituent Rcx;
wherein each Rwa, each Rwb, each Rza, each Rzb, each Rwx, each Rzx and each Rcx are each independently hydrogen, protium, deuterium, tritium, halogen, —NO2, —CN, —ORr, —SRr, —N(Rra)(R^{rb}), —C(O)Rr, —CO2Rr, —C(O)C(O)Rr, —C(O)CH2C(O)Rr, —S(O)Rr, —S(O)2Rr, —C(O)N(Rra)(R^{rb}), —SO2N(Rra)(R^{rb}), —OC(O)Rr, —N(R)SO2Rr, or a C1-6 aliphatic group optionally substituted with Rr;
wherein each Rr, each Rra and each R^{rb} are each independently hydrogen, protium, deuterium, tritium, halogen, —NO2, —CN, —OH, —SH, —NH2, —C(O)H, —CO2H, —C(O)C(O)H, —C(O)CH2C(O)H, —S(O)H, —S(O)2H, —C(O)NH2, —SO2NH2, —OC(O)H, —N(H)SO2H or a C1-6 aliphatic group;
-Lb- represents a peptide residue consisting of 2 to 7 amino acids;
-Lc- is selected from the group consisting of:

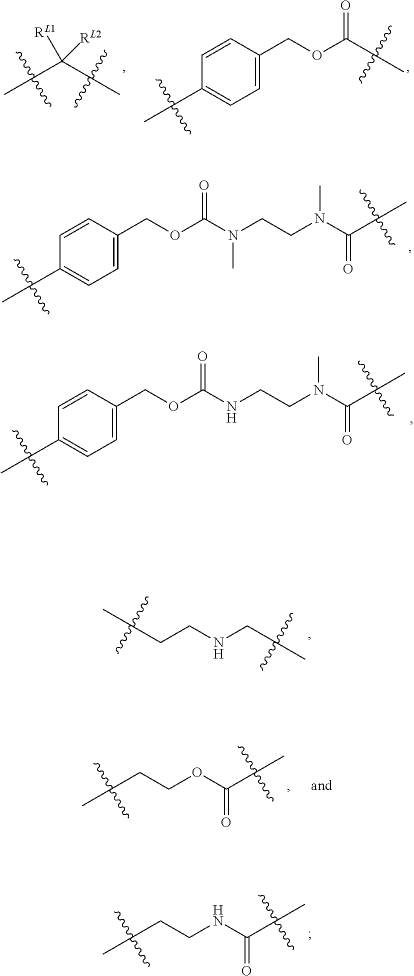

wherein RL1 and RL2 are each independently selected from the group consisting of hydrogen, protium, deuterium, tritium, halogen, —NO2, —CN, —OH, —SH, —NH2, —C(O)H, —CO2H, —C(O)C(O)H, —C(O)CH2C(O)H, —S(O)H, —S(O)2H, —C(O)NH2, —SO2NH2, —OC(O)H, —N(H)SO2H and a C1-6 aliphatic group;
U is O or S; and
V is (C1-6) alkylene, (C3-10) cycloalkylene, or (C1-6) alkylene-(C3-10) cycloalkylene.

In one embodiment, the antibody-drug conjugate has a structure shown as formula (III-D):

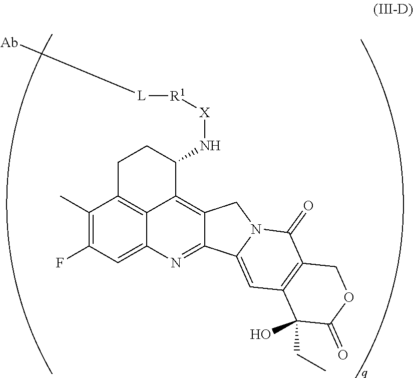

or a tautomer, mesomer, enantiomer or diastereoisomer thereof, or a pharmaceutically acceptable salt thereof wherein:
Ab is an antibody or fragment thereof that specifically binds to a target expressed on the surface of a tumour cell, preferably an antibody or a fragment thereof that specifically binds to B7H3, the connection number q is an integer from 1 to 16, and L, R1 and X is as defined above for the linker-payload.

In one embodiment, L is -La-Lb-Lc- wherein:
-La- is

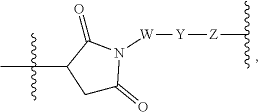

wherein W is —(C(Rwa)(Rwb))wn-, Y is —(OCH2CH2)yn-Oyp-, and Z is —(C(Rza)(Rzb))zn; wherein wn is 1, 2, 3 or 6, and
1 methylene unit of W is independently replaced by -Cyr-, —N(Rwx)C(O)—, —C(O)N(Rwx)- or —C(O)—;
wherein yn is 0, 4 or 8, and yp is 0 or 1;
wherein zn is 1, 2 or 3, and
1 methylene unit of Z is independently replaced by -Cyr-, —N(Rzx)C(O)—, —C(O)N(Rzx)- or —C(O)—;
-Cyr- is 3-10 membered saturated carbocyclylene, wherein -Cyr- is unsubstituted or independently substituted with 1 to 3 substituent Rex;
wherein each R^{wa}, each Rwb, each Rza, each Rzb, each Rwx, each Rzx and each Rcx are
each independently hydrogen, halogen, —ORr, or a C1-6 aliphatic group optionally substituted with Rr;
wherein each Rr is independently hydrogen, halogen or a C1-6 aliphatic group;
-Lb- is selected from the group consisting of:

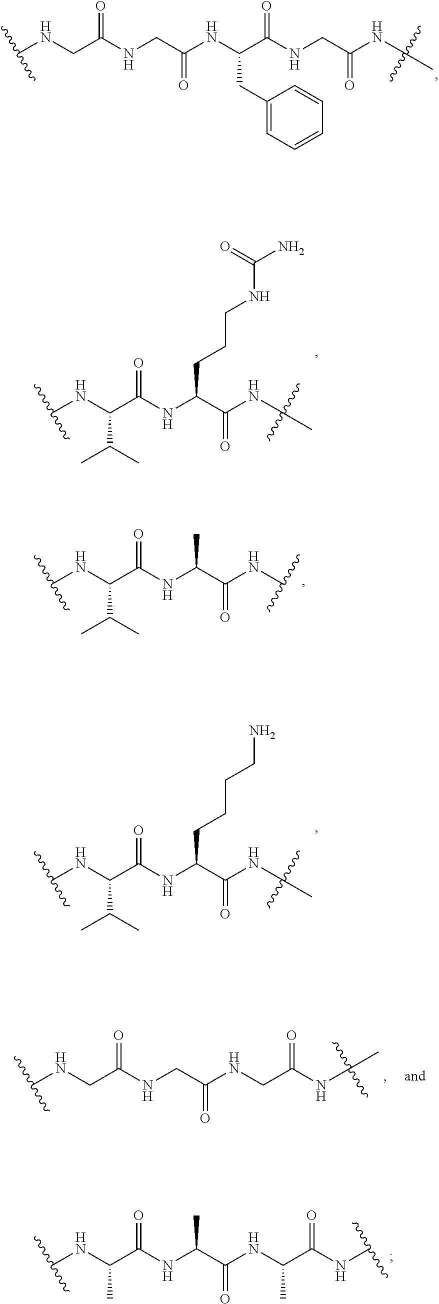 [SMILES: *NCC(=O)NCC(=O)N[C@@H](Cc1ccccc1)C(=O)NCC(=O)N* ; *NC(=O)[C@H](CCCNC(N)=O)NC(=O)[C@@H](N*)C(C)C ; *NC(=O)[C@H](C)NC(=O)[C@@H](N*)C(C)C ; *NC(=O)[C@H](CCCCN)NC(=O)[C@@H](N*)C(C)C ; *NCC(=O)NCC(=O)NCC(=O)N* ; *NC(=O)[C@H](C)NC(=O)[C@H](C)NC(=O)[C@H](C)N*]

-Lc- is

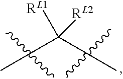

wherein RL1 and RL2 are each independently selected from the group consisting of hydrogen, halogen, —OH and a C1-6 aliphatic group.

In one embodiment, -La- is

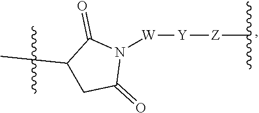

wherein W is —(C(Rwa)(Rwb))wn-, Y is —(OCH2CH2)yn-Oyp-, and Z is —(C(Rza)(Rzb))zn;
wherein wn is 1, 2, 3 or 6, and
1 methylene unit of W is independently replaced by -Cyr-, —N(Rwx)C(O)—, —C(O)N(Rwx)- or —C(O)—;
wherein yn is 0, 4 or 8, and yp is 0 or 1;
wherein zn is 1, 2 or 3, and
1 methylene unit of Z is independently replaced by -Cyr-, —N(Rzx)C(O)—, —C(O)N(Rzx)- or —C(O)—;
-Cyr- is 3-10 membered saturated carbocyclylene, wherein -Cyr- is unsubstituted or independently substituted with 1 to 3 substituent Rcx;
wherein each Rwa, each Rwb, each Rza, each Rzb, each Rwx, each Rzx and each Rcx are each independently hydrogen, halogen, —ORr, or a C1-6 aliphatic group optionally substituted with Rr;
wherein each Rr is independently hydrogen, halogen or a C1-6 aliphatic group;
-Lb- is

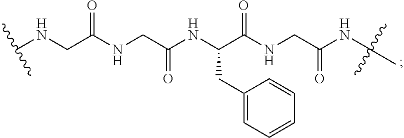 [SMILES: *NCC(=O)NCC(=O)N[C@@H](Cc1ccccc1)C(=O)NCC(=O)N*]

-Lc- is

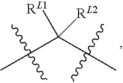

wherein RL1 and RL2 are each independently selected from the group consisting of hydrogen, halogen, —OH and a C1-6 aliphatic group.

Antibody-drug conjugates having the above structures are described in WO2022/068878 and national applications deriving therefrom.

In one embodiment, the antibody-drug conjugate has the formula (III-X):

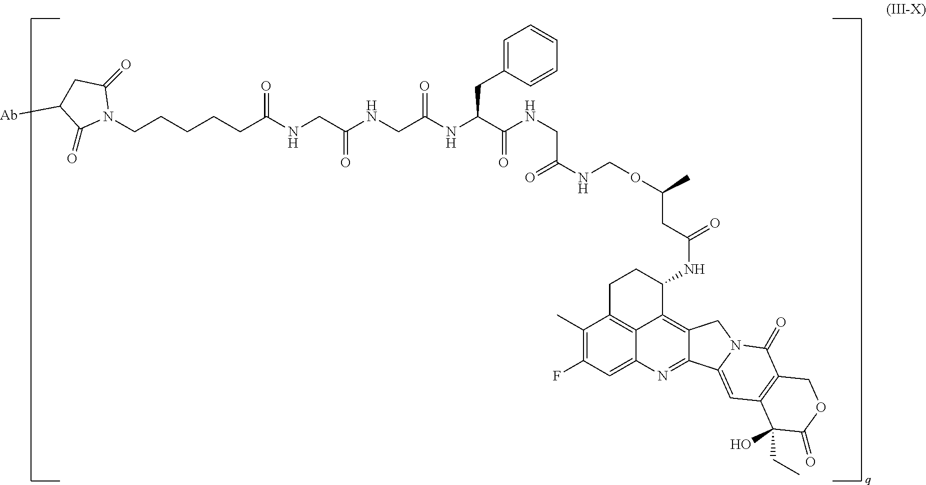

or a pharmaceutically acceptable salt thereof, wherein,
q represents a connection number, and q is selected from the group consisting of integers from 1 to 12, preferably integers from 2 to 8, more preferably integers from 3 to 7, for example, p is an integer from 4 to 6 or from 6 to 8, preferably 4, 5 or 6; and
Ab is an antibody or fragment thereof as defined herein.

Antibody-drug conjugates having the above structures are described in WO2022/068878 and national applications deriving therefrom.

### Example Anti-TROP2 and Anti-B7H3 Antibody-Drug Conjugates

In one embodiment, the antibody-drug conjugate has the following structure:

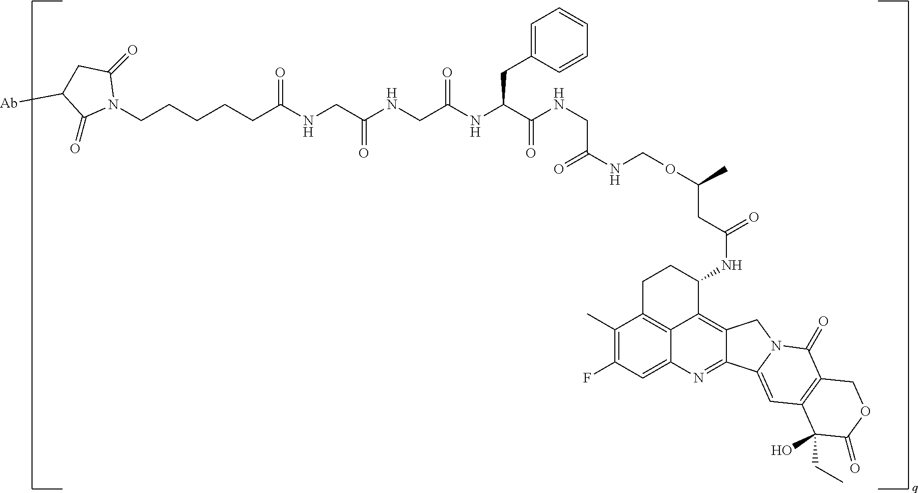

or a pharmaceutically acceptable salt thereof, wherein,
q represents a connection number, and q is selected from the group consisting of 1 to 12, preferably integers from 2 to 8, more preferably integers from 3 to 7, for example, q is an integer from 4 to 6 or from 6 to 8, preferably 4, 5 or 6; and
Ab is an anti-B7H3 antibody or fragment thereof, or an anti-TROP2 antibody or fragment thereof, as defined herein.

### Example Anti-TROP2 Antibody-Drug Conjugates

In one embodiment, the antibody-drug conjugate comprises an antibody or fragment thereof that specifically binds to TROP2.

In some embodiments, the antibody-drug conjugate comprises an anti-TROP2 antibody or fragment thereof comprising:
a heavy chain variable region comprising: (i) a HCDR1 comprising or consisting of the amino acid sequence of SEQ ID NO: 33; (ii) a HCDR2 comprising or consisting of the amino acid sequence of SEQ ID NO: 34; and (iii) a HCDR3 comprising or consisting of the amino acid sequence of SEQ ID NO: 35; and a light chain variable region comprising: (i) a LCDR1 comprising or consisting of the amino acid sequence of SEQ ID NO: 36; (ii) a LCDR2 comprising or consisting of the amino acid sequence of SEQ ID NO: 37; and (iii) a LCDR3 comprising or consisting of the amino acid sequence of SEQ ID NO: 38, wherein the CDRs are defined according to the Kabat numbering system; and/or
a heavy chain variable region comprising: (i) a HCDR1 comprising or consisting of the amino acid sequence of SEQ ID NO: 39; (ii) a HCDR2 comprising or consisting of the amino acid sequence of SEQ ID NO: 40; and (iii) a HCDR3 comprising or consisting of the amino acid sequence of SEQ ID NO: 41; and a light chain variable region comprising: (i) a LCDR1 comprising or consisting of the amino acid sequence of SEQ ID NO: 42; (ii) a LCDR2 comprising or consisting of the amino acid sequence of SEQ ID NO: 43; and (iii) a LCDR3 comprising or consisting of the amino acid sequence of SEQ ID NO: 44, wherein the CDRs are defined according to the IMGT numbering system.

In some embodiments, the antibody-drug conjugate comprises an anti-TROP2 antibody or fragment thereof comprising a heavy chain variable region comprising or consisting of the amino acid sequence of SEQ ID NO: 45 or an amino acid sequence having at least 80%, 85%, 90%, 95%, 96%, 97%, 98%, or 99% identity thereto; and a light chain variable region comprising or consisting of the amino acid sequence of SEQ ID NO: 46 or an amino acid sequence having at least 80%, 85%, 90%, 95%, 96%, 97%, 98%, or 99% identity thereto.

In some embodiments, the antibody-drug conjugate comprises an anti-TROP2 antibody or fragment thereof comprising a heavy chain comprising or consisting of the amino acid sequence of SEQ ID NO: 47 or an amino acid sequence having at least 80%, 85%, 90%, 95%, 96%, 97%, 98%, or 99% identity thereto; and a light chain comprising or consisting of the amino acid sequence of SEQ ID NO: 48 or an amino acid sequence having at least 80%, 85%, 90%, 95%, 96%, 97%, 98%, or 99% identity thereto.

In some embodiments, the antibody-drug conjugate comprises an anti-TROP2 antibody or fragment thereof comprising a heavy chain comprising or consisting of the amino acid sequence of SEQ ID NO: 47; and a light chain comprising or consisting of the amino acid sequence of SEQ ID NO: 48.

In one embodiment, the antibody-drug conjugate comprises sacituzumab or a fragment thereof.

In one embodiment, the antibody-drug conjugate has the structural formula (III-S):

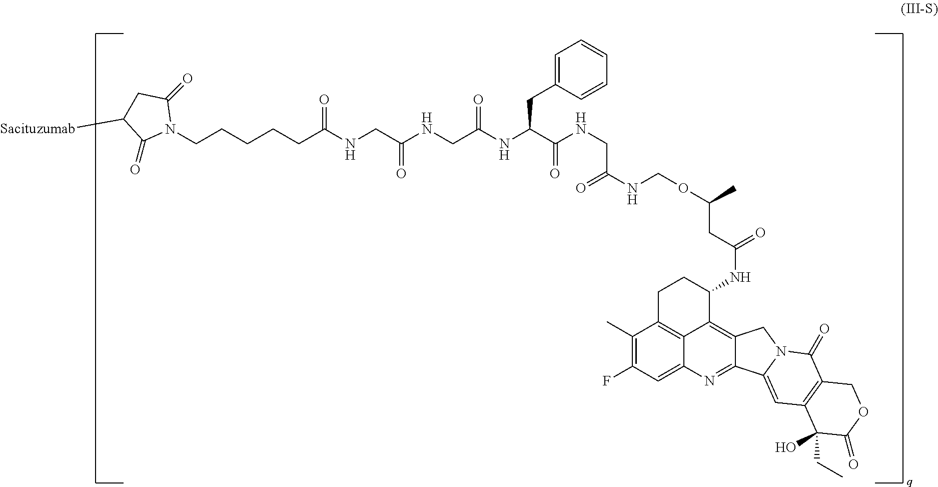

wherein q represents a connection number, and q is selected from the group consisting of 1 to 8, preferably integers from 2 to 6, more preferably integers from 3 to 5, for example, q is an integer of 3, 4 or 5. “Sacituzumab” may refer to an anti-TROP2 antibody comprising a heavy chain amino acid sequence as set forth in SEQ ID NO: 47 and a light chain amino acid sequence as set forth in SEQ ID NO: 48.

An antibody-drug conjugate of formula (III-S) where q is 4 is referred to herein as “Compound 3”. It is disclosed as compound ADC-III-28 in WO2022/068878 and national applications deriving therefrom.

### Example Anti-B7H3 Antibody-Drug Conjugates

In one embodiment, the antibody-drug conjugate comprises an antibody or fragment thereof that specifically binds to B7H3.

In one embodiment, the antibody-drug conjugate comprises an anti-B7H3 antibody or fragment thereof comprising:
a heavy chain variable region comprising: (i) a HCDR1 comprising or consisting of the amino acid sequence of SEQ ID NO: 17; (ii) a HCDR2 comprising or consisting of the amino acid sequence of SEQ ID NO: 18; and (iii) a HCDR3 comprising or consisting of the amino acid sequence of SEQ ID NO: 19; and a light chain variable region comprising: (i) a LCDR1 comprising or consisting of the amino acid sequence of SEQ ID NO: 20; (ii) a LCDR2 comprising or consisting of the amino acid sequence of SEQ ID NO: 21; and (iii) a LCDR3 comprising or consisting of the amino acid sequence of SEQ ID NO: 22, wherein the CDRs are defined according to the Kabat numbering system; and/or
a heavy chain variable region comprising: (i) a HCDR1 comprising or consisting of the amino acid sequence of SEQ ID NO: 23; (ii) a HCDR2 comprising or consisting of the amino acid sequence of SEQ ID NO: 24; and (iii) a HCDR3 comprising or consisting of the amino acid sequence of SEQ ID NO: 25; and a light chain variable region comprising: (i) a LCDR1 comprising or consisting of the amino acid sequence of SEQ ID NO: 26; (ii) a LCDR2 comprising or consisting of the amino acid sequence of SEQ ID NO: 27; and (iii) a LCDR3 comprising or consisting of the amino acid sequence of SEQ ID NO: 28, wherein the CDRs are defined according to the IMGT numbering system.

In one embodiment, the antibody-drug conjugate comprises an anti-B7H3 antibody or fragment thereof comprising a heavy chain variable region comprising or consisting of the amino acid sequence of SEQ ID NO: 29, or an amino acid sequence having at least 80%, 85%, 90%, 95%, 96%, 97%, 98%, or 99% identity thereto; and a light chain variable region comprising or consisting of the amino acid sequence of SEQ ID NO: 30 or an amino acid sequence having at least 80%, 85%, 90%, 95%, 96%, 97%, 98%, or 99% identity thereto.

In one embodiment, the antibody-drug conjugate comprises an anti-B7H3 antibody or fragment thereof comprising a heavy chain comprising or consisting of the amino acid sequence of the amino acid sequence of SEQ ID NO: 31, or an amino acid sequence having at least 80%, 85%, 90%, 95%, 96%, 97%, 98%, or 99% identity thereto; and a light chain comprising or consisting of the amino acid sequence of SEQ ID NO: 32 or an amino acid sequence having at least 80%, 85%, 90%, 95%, 96%, 97%, 98%, or 99% identity thereto.

In one embodiment, the antibody-drug conjugate comprises an anti-B7H3 antibody or fragment thereof comprising a heavy chain comprising or consisting of the amino acid sequence of the amino acid sequence of SEQ ID NO: 31; and a light chain comprising or consisting of the amino acid sequence of SEQ ID NO: 32.

In one embodiment, the antibody-drug conjugate has the structural formula (III-W):
wherein,

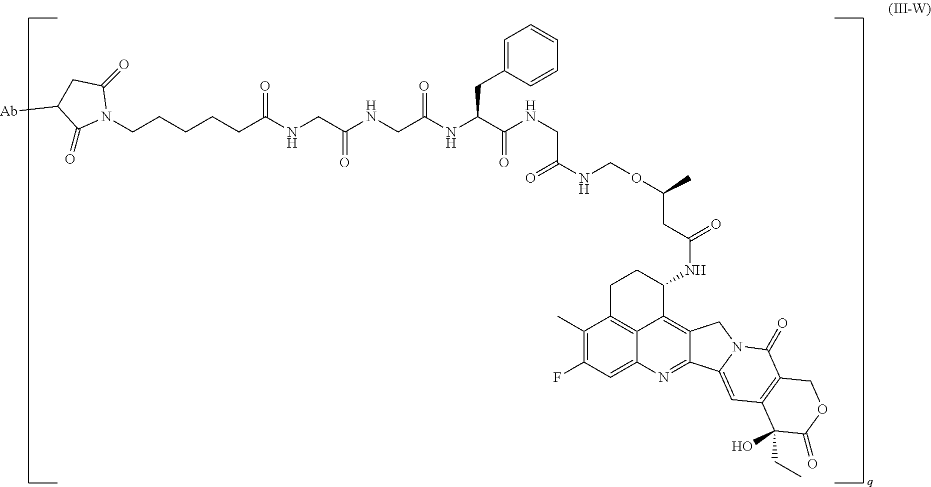

q represents a connection number, and q is selected from the group consisting of integers from 1 to 10, preferably integers from 2 to 8; for example, q is an integer from 4 to 8, for example, q is an integer from 5 to 7, preferably 6; and
Ab is an anti-B7H3 antibody comprising: a heavy chain amino acid sequence as set forth in SEQ ID NO: 31 and a light chain amino acid sequence as set forth in SEQ ID NO: 32.

An antibody-drug conjugate of formula (III-W) where q is 6 is referred to herein as “Compound 2”. It is disclosed as compound WBP301088-X2 in WO2023/236949 and national applications deriving therefrom.

### Example Anti-HER2 Antibody-Drug Conjugates

In one embodiment, the antibody-drug conjugate comprises a structure shown as formula (II-Dₓ):

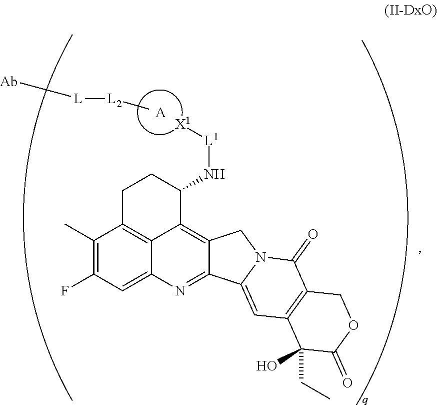

or a tautomer, a mesomer, an enantiomer or a diastereoisomer thereof, or a pharmaceutically acceptable salt thereof wherein, Ab is an antibody or fragment thereof, and a connection number q is an integer from 1 to 10, and L, L², A, X¹ and L¹ are as defined above.

In one embodiment, the antibody-drug conjugate comprises an antibody or fragment thereof that specifically binds to HER2.

In one embodiment, the antibody-drug conjugate comprises trastuzumab or a fragment thereof.

In one embodiment, the antibody-drug conjugate comprises pertuzumab or a fragment thereof.

In some embodiments, the antibody-drug conjugate comprises an anti-HER2 antibody or fragment thereof comprising:
(a) (i) a heavy chain variable region comprising: (i) a HCDR1 comprising or consisting of the amino acid sequence of SEQ ID NO: 49; (ii) a HCDR2 comprising or consisting of the amino acid sequence of SEQ ID NO: 50; and (iii) a HCDR3 comprising or consisting of the amino acid sequence of SEQ ID NO: 51; and a light chain variable region comprising: (i) a LCDR1 comprising or consisting of the amino acid sequence of SEQ ID NO: 52; (ii) a LCDR2 comprising or consisting of the amino acid sequence of SEQ ID NO: 53; and (iii) a LCDR3 comprising or consisting of the amino acid sequence of SEQ ID NO: 54, wherein the CDRs are defined according to the Kabat numbering system; and/or
(ii) a heavy chain variable region comprising: (i) a HCDR1 comprising or consisting of the amino acid sequence of SEQ ID NO: 55; (ii) a HCDR2 comprising or consisting of the amino acid sequence of SEQ ID NO: 56; and (iii) a HCDR3 comprising or consisting of the amino acid sequence of SEQ ID NO: 57; and a light chain variable region comprising: (i) a LCDR1 comprising or consisting of the amino acid sequence of SEQ ID NO: 58; (ii) a LCDR2 comprising or consisting of the amino acid sequence of SEQ ID NO: 59; and (iii) a LCDR3 comprising or consisting of the amino acid sequence of SEQ ID NO: 60, wherein the CDRs are defined according to the IMGT numbering system; or
(b) (i) a heavy chain variable region comprising: (i) a HCDR1 comprising or consisting of the amino acid sequence of SEQ ID NO: 65; (ii) a HCDR2 comprising or consisting of the amino acid sequence of SEQ ID NO: 66; and (iii) a HCDR3 comprising or consisting of the amino acid sequence of SEQ ID NO: 67; and a light chain variable region comprising: (i) a LCDR1 comprising or consisting of the amino acid sequence of SEQ ID NO: 68; (ii) a LCDR2 comprising or consisting of the amino acid sequence of SEQ ID NO: 69; and (iii) a LCDR3 comprising or consisting of the amino acid sequence of SEQ ID NO: 70, wherein the CDRs are defined according to the Kabat numbering system; and/or
(ii) a heavy chain variable region comprising: (i) a HCDR1 comprising or consisting of the amino acid sequence of SEQ ID NO: 71; (ii) a HCDR2 comprising or consisting of the amino acid sequence of SEQ ID NO: 72; and (iii) a HCDR3 comprising or consisting of the amino acid sequence of SEQ ID NO: 73; and a light chain variable region comprising: (i) a LCDR1 comprising or consisting of the amino acid sequence of SEQ ID NO: 74; (ii) a LCDR2 comprising or consisting of the amino acid sequence of SEQ ID NO: 75; and (iii) a LCDR3 comprising or consisting of the amino acid sequence of SEQ ID NO: 76, wherein the CDRs are defined according to the IMGT numbering system.

In some embodiments, the antibody-drug conjugate comprises an anti-HER2 antibody or fragment thereof comprising:
(a) a heavy chain variable region comprising or consisting of the amino acid sequence of SEQ ID NO: 61 or an amino acid sequence having at least 80%, 85%, 90%, 95%, 96%, 97%, 98%, or 99% identity thereto; and a light chain variable region comprising or consisting of the amino acid sequence of SEQ ID NO: 62 or an amino acid sequence having at least 80%, 85%, 90%, 95%, 96%, 97%, 98%, or 99% identity thereto; or
(b) a heavy chain variable region comprising or consisting of the amino acid sequence of SEQ ID NO: 77 or an amino acid sequence having at least 80%, 85%, 90%, 95%, 96%, 97%, 98%, or 99% identity thereto; and a light chain variable region comprising or consisting of the amino acid sequence of SEQ ID NO: 78 or an amino acid sequence having at least 80%, 85%, 90%, 95%, 96%, 97%, 98%, or 99% identity thereto.

In some embodiments, the antibody-drug conjugate comprises an anti-HER2 antibody or fragment thereof comprising:
(a) a heavy chain comprising or consisting of the amino acid sequence of SEQ ID NO: 63 or an amino acid sequence having at least 80%, 85%, 90%, 95%, 96%, 97%, 98%, or 99% identity thereto; and a light chain comprising or consisting of the amino acid sequence of SEQ ID NO: 64 or an amino acid sequence having at least 80%, 85%, 90%, 95%, 96%, 97%, 98%, or 99% identity thereto; or
(b) a heavy chain comprising or consisting of the amino acid sequence of SEQ ID NO: 79 or an amino acid sequence having at least 80%, 85%, 90%, 95%, 96%, 97%, 98%, or 99% identity thereto; and a light chain comprising or consisting of the amino acid sequence of SEQ ID NO: 80 or an amino acid sequence having at least 80%, 85%, 90%, 95%, 96%, 97%, 98%, or 99% identity thereto.

In some embodiments, the antibody-drug conjugate comprises an anti-HER2 antibody or fragment thereof comprising:
(a) a heavy chain comprising or consisting of the amino acid sequence of SEQ ID NO: 63; and a light chain comprising or consisting of the amino acid sequence of SEQ ID NO: 64; or
(b) a heavy chain comprising or consisting of the amino acid sequence of SEQ ID NO: 79; and a light chain comprising or consisting of the amino acid sequence of SEQ ID NO: 80.

In one embodiment, the antibody-drug conjugate has the following structure:

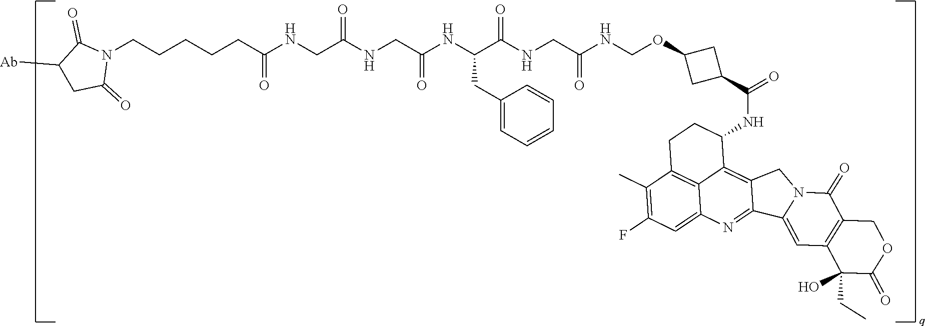

wherein,
q represents a connection number, and q is selected from the group consisting of integers from 1 to 16, preferably integers from 4 to 12, for example, q is an integer from 6 to 10, for example, q is an integer from 7 to 9, such as 7, 8 or 9, preferably 8; and
Ab is an anti-HER2 antibody or fragment thereof as defined herein.

In one embodiment, the antibody-drug conjugate has the structural formula (II-T):

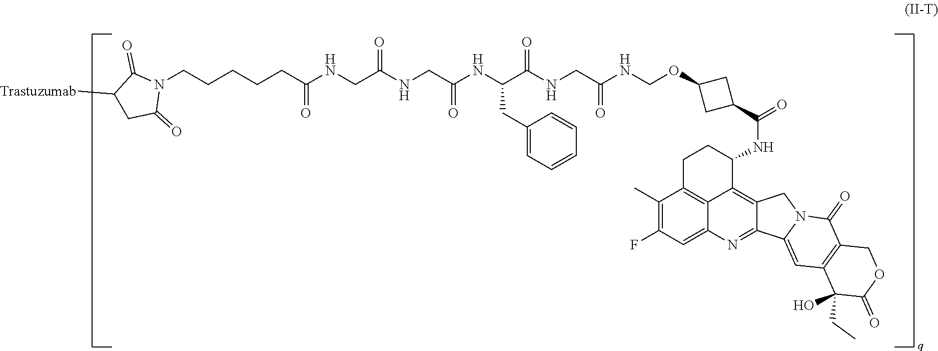

wherein, q is a connection number and q is selected from the group consisting of integers from 1 to 16, preferably integers from 4 to 12, for example, q is an integer from 6 to 10, for example, n is an integer from 7 to 9, such as 7, 8 or 9, preferably 8. “Trastuzumab” may refer to an anti-HER2 antibody comprising a heavy chain amino acid sequence as set forth in SEQ ID NO: 63 and a light chain amino acid sequence as set forth in SEQ ID NO: 64.

An antibody-drug conjugate of formula (II-T) where q is 8 is referred to herein as “Compound 1”. It is disclosed as compound ADC-II-9 in WO2022/068878 and national applications deriving therefrom.

In one embodiment, the antibody-drug conjugate has a structure shown as formula (VI-A)

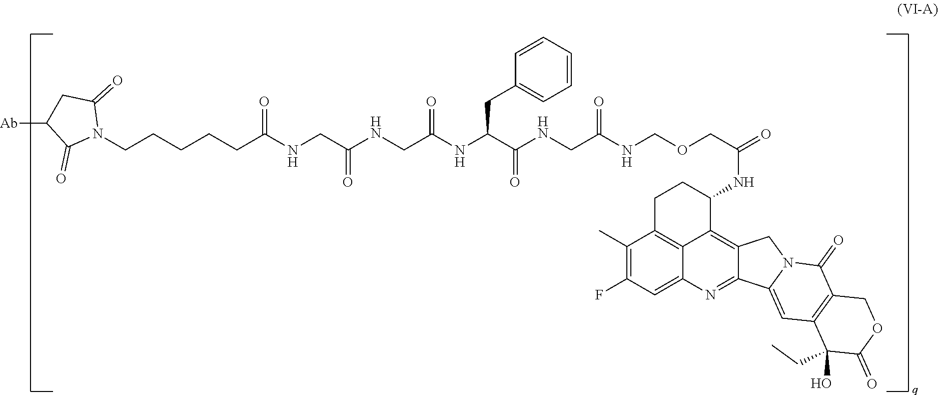

or a pharmaceutically acceptable salt thereof wherein:
Ab is the antibody or fragment thereof as defined herein; and
q is the connection number, and q is an integer from 1 to 16; preferably the connection number q is an integer from 4 to 12, more preferably q is an integer from 6 to 10; such as 7 to 9, such as 7, 8 or 9; preferably 8.

In this embodiment, the antibody or fragment thereof is preferably an anti-HER2 antibody or fragment thereof as defined herein. In one embodiment, the antibody-drug conjugate comprises trastuzumab or a fragment thereof. In one embodiment, the antibody-drug conjugate comprises trastuzumab.

In one embodiment, the antibody-drug conjugate has a structure shown as formula (VI-B)

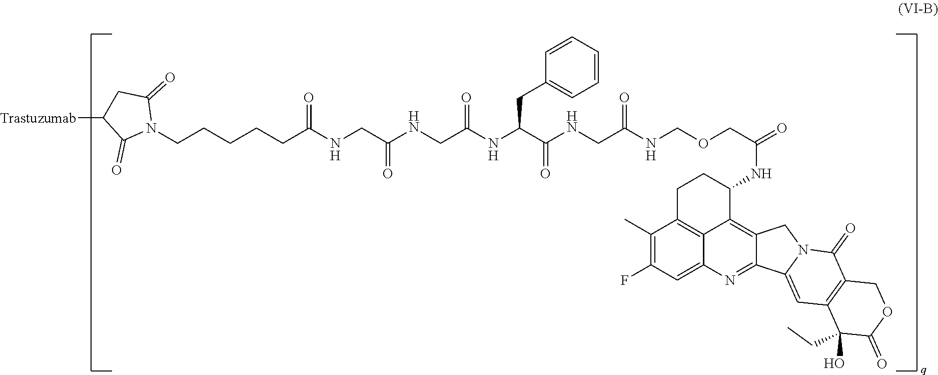

wherein, q is a connection number and q is selected from the group consisting of integers from 1 to 16, preferably integers from 4 to 12, for example, q is an integer from 6 to 10, for example, n is an integer from 7 to 9, such as 7, 8 or 9, preferably 8.

An antibody-drug conjugate of formula (VI-B) where q is 8 is referred to herein as “Compound 6” and may also be known as trastuzumab deruxtecan, Enhertu®, or DS-8201 (see e.g. Modi, S., et al., 2020. New England Journal of Medicine, 382(7), pp. 610-621). In one embodiment, the antibody-drug conjugate is trastuzumab deruxtecan.

### Example Anti-HER3 Antibody-Drug Conjugates

In one embodiment, the antibody-drug conjugate has the structure (I-M)

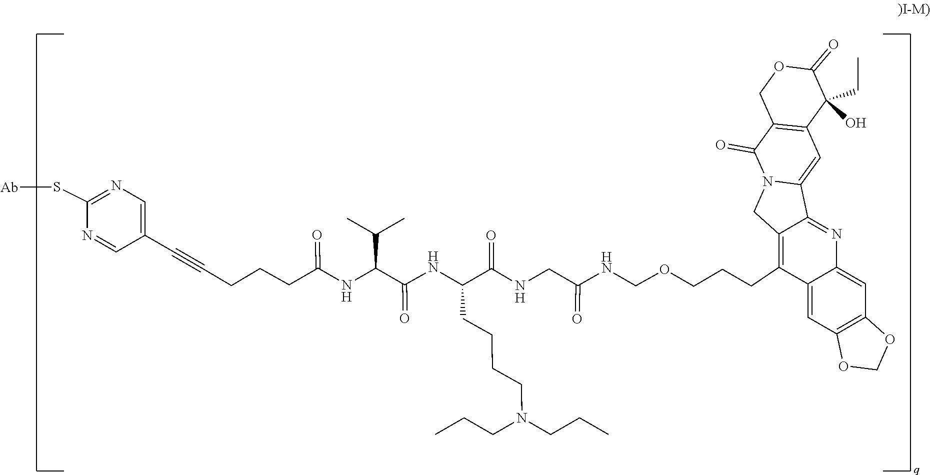

or a pharmaceutically acceptable salt thereof wherein:
Ab is the antibody or fragment thereof as defined herein; and
q is the connection number, and q is an integer from 1 to 16; preferably the connection number q is an integer from 4 to 12, more preferably q is an integer from 6 to 10; such as 7 to 9, such as 7, 8 or 9; preferably 8.

In this embodiment, the antibody or fragment thereof is preferably an anti-HER3 antibody or fragment thereof.

In one embodiment, the antibody-drug conjugate comprises an anti-HER3 antibody or fragment thereof comprising:
a heavy chain variable region comprising: (i) a HCDR1 comprising or consisting of the amino acid sequence of SEQ ID NO: 190; (ii) a HCDR2 comprising or consisting of the amino acid sequence of SEQ ID NO: 191; and (iii) a HCDR3 comprising or consisting of the amino acid sequence of SEQ ID NO: 192; and a light chain variable region comprising: (i) a LCDR1 comprising or consisting of the amino acid sequence of SEQ ID NO: 193; (ii) a LCDR2 comprising or consisting of the amino acid sequence of SEQ ID NO: 194; and (iii) a LCDR3 comprising or consisting of the amino acid sequence of SEQ ID NO: 195, wherein the CDRs are defined according to the Kabat numbering system; and/or
a heavy chain variable region comprising: (i) a HCDR1 comprising or consisting of the amino acid sequence of SEQ ID NO: 196; (ii) a HCDR2 comprising or consisting of the amino acid sequence of SEQ ID NO: 197; and (iii) a HCDR3 comprising or consisting of the amino acid sequence of SEQ ID NO: 198; and a light chain variable region comprising: (i) a LCDR1 comprising or consisting of the amino acid sequence of SEQ ID NO: 199; (ii) a LCDR2 comprising or consisting of the amino acid sequence of SEQ ID NO: 200; and (iii) a LCDR3 comprising or consisting of the amino acid sequence of SEQ ID NO: 201, wherein the CDRs are defined according to the IMGT numbering system.

In one embodiment, the antibody-drug conjugate comprises an anti-HER3 antibody or fragment thereof comprising a heavy chain variable region comprising or consisting of the amino acid sequence of SEQ ID NO: 202, or an amino acid sequence having at least 80%, 85%, 90%, 95%, 96%, 97%, 98%, or 99% identity thereto; and a light chain variable region comprising or consisting of the amino acid sequence of SEQ ID NO: 203 or an amino acid sequence having at least 80%, 85%, 90%, 95%, 96%, 97%, 98%, or 99% identity thereto.

In one embodiment, the antibody-drug conjugate comprises an anti-HER3 antibody or fragment thereof comprising a heavy chain comprising or consisting of the amino acid sequence of the amino acid sequence of SEQ ID NO: 204, or an amino acid sequence having at least 80%, 85%, 90%, 95%, 96%, 97%, 98%, or 99% identity thereto; and a light chain comprising or consisting of the amino acid sequence of SEQ ID NO: 205 or an amino acid sequence having at least 80%, 85%, 90%, 95%, 96%, 97%, 98%, or 99% identity thereto.

In one embodiment, the antibody-drug conjugate comprises an anti-HER3 antibody or fragment thereof comprising a heavy chain comprising or consisting of the amino acid sequence of the amino acid sequence of SEQ ID NO: 204; and a light chain comprising or consisting of the amino acid sequence of SEQ ID NO: 205.

An antibody-drug conjugate of formula (I-M) where the antibody portion is an anti-HER3 antibody comprising a heavy chain amino acid sequence as set forth in SEQ ID NO: 204 and a light chain amino acid sequence as set forth in SEQ ID NO: 205 and q is 8 is referred to herein as “Compound 4”. It is disclosed as compound HER3-ADC-07 in WO2022/170971 and national applications deriving therefrom.

### Example Vedotin-Based Antibody Drug Conjugates

In one embodiment, the antibody-drug conjugate is of the formula (VIIb):

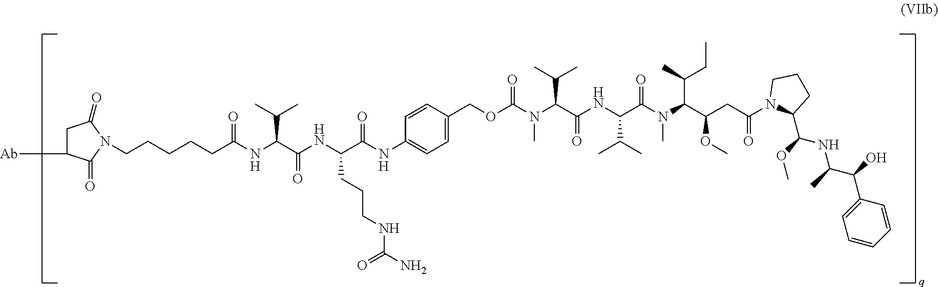

or a pharmaceutically acceptable salt thereof, wherein,
q represents a connection number, and q is selected from the group consisting of 1 to 16, and Ab is an antibody or fragment thereof, as defined herein.

In some embodiments, Ab is an anti-CD30 antibody or fragment thereof, an anti-Nectin-4 antibody or fragment thereof, an anti-TF antibody or fragment thereof, an anti-B7-H4 antibody or fragment thereof, an anti-ITGB6 antibody or fragment thereof, or an anti-HER2 antibody or fragment thereof. In some embodiments, Ab is an anti-CD30 antibody, an anti-Nectin-4 antibody, an anti-TF antibody, an anti-B7-H4 antibody, an anti-ITGB6 antibody, or an anti-HER2 antibody.

In some embodiments, Ab is Brentuximab or a fragment thereof, Enfortumab or a fragment thereof, Tisotumab or a fragment thereof, Felmetatug or a fragment thereof, Sigvotatug or a fragment thereof, or Disitamab or a fragment thereof. In some embodiments, Ab is Brentuximab, Enfortumab, Tisotumab, Felmetatug, Sigvotatug, or Disitamab.

### Example Anti-CD30 Antibody Drug Conjugates

In one embodiment, the antibody-drug conjugate is of the formula (VIIb):

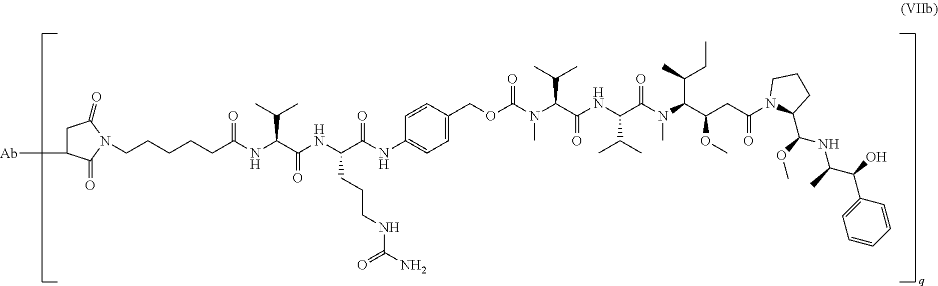

or a pharmaceutically acceptable salt thereof, wherein,
q represents a connection number, and q is selected from the group consisting of 1 to 16 preferably integers from 2 to 8; for example, q is an integer from 3 to 5, preferably 4, and
Ab is an anti-CD30 antibody or fragment thereof, as defined herein.

In one embodiment, the antibody-drug conjugate comprises an anti-CD30 antibody or fragment thereof comprising: a heavy chain variable region comprising: (i) a HCDR1 comprising or consisting of the amino acid sequence of SEQ ID NO: 234; (ii) a HCDR2 comprising or consisting of the amino acid sequence of SEQ ID NO: 235; and (iii) a HCDR3 comprising or consisting of the amino acid sequence of SEQ ID NO: 236; and a light chain variable region comprising: (i) a LCDR1 comprising or consisting of the amino acid sequence of SEQ ID NO: 237; (ii) a LCDR2 comprising or consisting of the amino acid sequence of SEQ ID NO: 238; and (iii) a LCDR3 comprising or consisting of the amino acid sequence of SEQ ID NO: 239, wherein the CDRs are defined according to the Kabat numbering system; and/or a heavy chain variable region comprising: (i) a HCDR1 comprising or consisting of the amino acid sequence of SEQ ID NO: 240; (ii) a HCDR2 comprising or consisting of the amino acid sequence of SEQ ID NO: 241; and (iii) a HCDR3 comprising or consisting of the amino acid sequence of SEQ ID NO: 242; and a light chain variable region comprising: (i) a LCDR1 comprising or consisting of the amino acid sequence of SEQ ID NO: 243; (ii) a LCDR2 comprising or consisting of the amino acid sequence of SEQ ID NO: 244; and (iii) a LCDR3 comprising or consisting of the amino acid sequence of SEQ ID NO: 245, wherein the CDRs are defined according to the IMGT numbering system.

In one embodiment, the antibody-drug conjugate comprises an anti-CD30 antibody or fragment thereof comprising a heavy chain variable region comprising or consisting of the amino acid sequence of SEQ ID NO: 246, or an amino acid sequence having at least 80%, 85%, 90%, 95%, 96%, 97%, 98%, or 99% identity thereto; and a light chain variable region comprising or consisting of the amino acid sequence of SEQ ID NO: 247 or an amino acid sequence having at least 80%, 85%, 90%, 95%, 96%, 97%, 98%, or 99% identity thereto.

In one embodiment, the antibody-drug conjugate comprises an anti-CD30 antibody or fragment thereof comprising a heavy chain comprising or consisting of the amino acid sequence of the amino acid sequence of SEQ ID NO: 248, or an amino acid sequence having at least 80%, 85%, 90%, 95%, 96%, 97%, 98%, or 99% identity thereto; and a light chain comprising or consisting of the amino acid sequence of SEQ ID NO: 249 or an amino acid sequence having at least 80%, 85%, 90%, 95%, 96%, 97%, 98%, or 99% identity thereto.

In one embodiment, the antibody-drug conjugate comprises an anti-CD30 antibody or fragment thereof comprising a heavy chain comprising or consisting of the amino acid sequence of the amino acid sequence of SEQ ID NO: 248; and a light chain comprising or consisting of the amino acid sequence of SEQ ID NO: 249.

In one embodiment, the antibody-drug conjugate comprises Brentuximab or a fragment thereof. In one embodiment, the antibody-drug conjugate comprises Brentuximab.

An antibody-drug conjugate of formula (VIIb) where q is 4 and Ab is Brentuximab is referred to herein as “Compound 7” and may also be known as Brentuximab vedotin. In one embodiment, the antibody-drug conjugate is Brentuximab vedotin.

### Example Anti-Nectin-4 Antibody Drug Conjugates

In one embodiment, the antibody-drug conjugate is of the formula (VIIb):

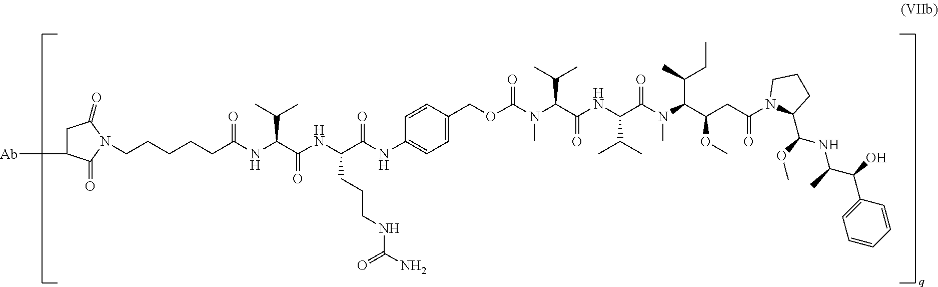

or a pharmaceutically acceptable salt thereof, wherein,
q represents a connection number, and q is selected from the group consisting of 1 to 16 preferably integers from 2 to 8; for example, q is an integer from 3 to 5, preferably 4, and
Ab is an anti-Nectin-4 antibody or fragment thereof, as defined herein.

In one embodiment, the antibody-drug conjugate comprises an anti-Nectin-4 antibody or fragment thereof comprising: a heavy chain variable region comprising: (i) a HCDR1 comprising or consisting of the amino acid sequence of SEQ ID NO: 250; (ii) a HCDR2 comprising or consisting of the amino acid sequence of SEQ ID NO: 251, and (iii) a HCDR3 comprising or consisting of the amino acid sequence of SEQ ID NO: 252, and a light chain variable region comprising: (i) a LCDR1 comprising or consisting of the amino acid sequence of SEQ ID NO: 253; (ii) a LCDR2 comprising or consisting of the amino acid sequence of SEQ ID NO: 254; and (iii) a LCDR3 comprising or consisting of the amino acid sequence of SEQ ID NO: 255, wherein the CDRs are defined according to the Kabat numbering system; and/or a heavy chain variable region comprising: (i) a HCDR1 comprising or consisting of the amino acid sequence of SEQ ID NO: 256; (ii) a HCDR2 comprising or consisting of the amino acid sequence of SEQ ID NO: 257; and (iii) a HCDR3 comprising or consisting of the amino acid sequence of SEQ ID NO: 258; and a light chain variable region comprising: (i) a LCDR1 comprising or consisting of the amino acid sequence of SEQ ID NO: 259; (ii) a LCDR2 comprising or consisting of the amino acid sequence of SEQ ID NO: 260; and (iii) a LCDR3 comprising or consisting of the amino acid sequence of SEQ ID NO: 261, wherein the CDRs are defined according to the IMGT numbering system.

In one embodiment, the antibody-drug conjugate comprises an anti-Nectin-4 antibody or fragment thereof comprising a heavy chain variable region comprising or consisting of the amino acid sequence of SEQ ID NO: 262, or an amino acid sequence having at least 80%, 85%, 90%, 95%, 96%, 97%, 98%, or 99% identity thereto; and a light chain variable region comprising or consisting of the amino acid sequence of SEQ ID NO: 263 or an amino acid sequence having at least 80%, 85%, 90%, 95%, 96%, 97%, 98%, or 99% identity thereto.

In one embodiment, the antibody-drug conjugate comprises an anti-Nectin-4 antibody or fragment thereof comprising a heavy chain comprising or consisting of the amino acid sequence of the amino acid sequence of SEQ ID NO: 264, or an amino acid sequence having at least 80%, 85%, 90%, 95%, 96%, 97%, 98%, or 99% identity thereto; and a light chain comprising or consisting of the amino acid sequence of SEQ ID NO: 265 or an amino acid sequence having at least 80%, 85%, 90%, 95%, 96%, 97%, 98%, or 99% identity thereto.

In one embodiment, the antibody-drug conjugate comprises an anti-Nectin-4 antibody or fragment thereof comprising a heavy chain comprising or consisting of the amino acid sequence of the amino acid sequence of SEQ ID NO: 264; and a light chain comprising or consisting of the amino acid sequence of SEQ ID NO: 265.

In one embodiment, the antibody-drug conjugate comprises Enfortumab or a fragment thereof. In one embodiment, the antibody-drug conjugate comprises Enfortumab.

An antibody-drug conjugate of formula (VIIb) where q is 4 and Ab is Enfortumab is referred to herein as “Compound 8” and may also be known as Enfortumab vedotin. In one embodiment, the antibody-drug conjugate is Enfortumab vedotin.

### Example Anti-TF Antibody Drug Conjugates

In one embodiment, the antibody-drug conjugate is of the formula (VIIb):

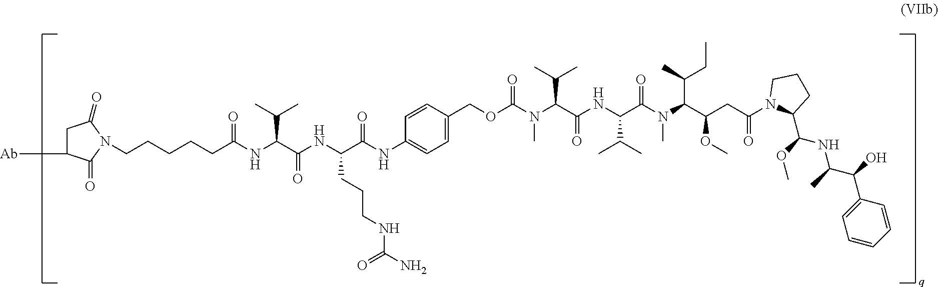

or a pharmaceutically acceptable salt thereof, wherein,
q represents a connection number, and q is selected from the group consisting of 1 to 16 preferably integers from 2 to 8; for example, q is an integer from 3 to 5, preferably 4, and
Ab is an anti-TF antibody or fragment thereof, as defined herein.

In one embodiment, the antibody-drug conjugate comprises an anti-TF antibody or fragment thereof comprising: a heavy chain variable region comprising: (i) a HCDR1 comprising or consisting of the amino acid sequence of SEQ ID NO: 266; (ii) a HCDR2 comprising or consisting of the amino acid sequence of SEQ ID NO: 267, and (iii) a HCDR3 comprising or consisting of the amino acid sequence of SEQ ID NO: 268, and a light chain variable region comprising: (i) a LCDR1 comprising or consisting of the amino acid sequence of SEQ ID NO: 269; (ii) a LCDR2 comprising or consisting of the amino acid sequence of SEQ ID NO: 270; and (iii) a LCDR3 comprising or consisting of the amino acid sequence of SEQ ID NO: 271, wherein the CDRs are defined according to the Kabat numbering system; and/or a heavy chain variable region comprising: (i) a HCDR1 comprising or consisting of the amino acid sequence of SEQ ID NO: 272; (ii) a HCDR2 comprising or consisting of the amino acid sequence of SEQ ID NO: 273; and (iii) a HCDR3 comprising or consisting of the amino acid sequence of SEQ ID NO: 274; and a light chain variable region comprising: (i) a L CDR1 comprising or consisting of the amino acid sequence of SEQ ID NO: 275; (ii) a LCDR2 comprising or consisting of the amino acid sequence of SEQ ID NO: 276; and (iii) a LCDR3 comprising or consisting of the amino acid sequence of SEQ ID NO: 277, wherein the CDRs are defined according to the IMGT numbering system.

In one embodiment, the antibody-drug conjugate comprises an anti-TF antibody or fragment thereof comprising a heavy chain variable region comprising or consisting of the amino acid sequence of SEQ ID NO: 278, or an amino acid sequence having at least 80%, 85%, 90%, 95%, 96%, 97%, 98%, or 99% identity thereto; and a light chain variable region comprising or consisting of the amino acid sequence of SEQ ID NO: 279 or an amino acid sequence having at least 80%, 85%, 90%, 95%, 96%, 97%, 98%, or 99% identity thereto.

In one embodiment, the antibody-drug conjugate comprises an anti-TF antibody or fragment thereof comprising a heavy chain comprising or consisting of the amino acid sequence of the amino acid sequence of SEQ ID NO: 280, or an amino acid sequence having at least 80%, 85%, 90%, 95%, 96%, 97%, 98%, or 99% identity thereto; and a light chain comprising or consisting of the amino acid sequence of SEQ ID NO: 281 or an amino acid sequence having at least 80%, 85%, 90%, 95%, 96%, 97%, 98%, or 99% identity thereto.

In one embodiment, the antibody-drug conjugate comprises an anti-TF antibody or fragment thereof comprising a heavy chain comprising or consisting of the amino acid sequence of the amino acid sequence of SEQ ID NO: 280; and a light chain comprising or consisting of the amino acid sequence of SEQ ID NO: 281.

In one embodiment, the antibody-drug conjugate comprises Tisotumab or a fragment thereof. In one embodiment, the antibody-drug conjugate comprises Tisotumab.

An antibody-drug conjugate of formula (VIIb) where q is 4 and Ab is Tisotumab is referred to herein as “Compound 9” and may also be known as Tisotumab vedotin. In one embodiment, the antibody-drug conjugate is Tisotumab vedotin.

### Example Anti-B7-H4 Antibody Drug Conjugates

In one embodiment, the antibody-drug conjugate is of the formula (VIIb):

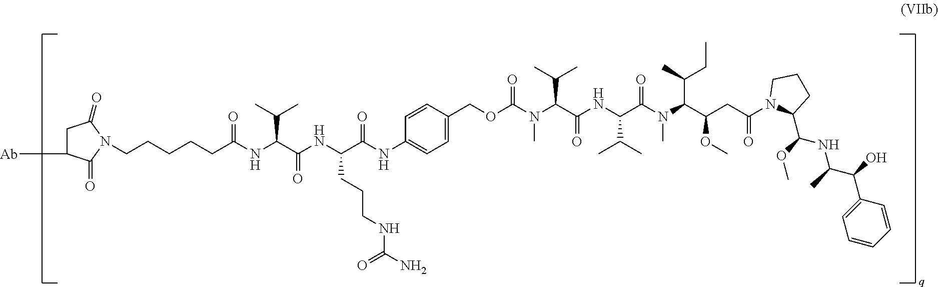

or a pharmaceutically acceptable salt thereof, wherein,
q represents a connection number, and q is selected from the group consisting of 1 to 16 preferably integers from 2 to 8; for example, q is an integer from 3 to 5, preferably 4, and
Ab is an anti-B7-H4 antibody or fragment thereof, as defined herein.

In one embodiment, the antibody-drug conjugate comprises an anti-B7-H4 antibody or fragment thereof comprising: a heavy chain variable region comprising: (i) a HCDR1 comprising or consisting of the amino acid sequence of SEQ ID NO: 282; (ii) a HCDR2 comprising or consisting of the amino acid sequence of SEQ ID NO: 283, and (iii) a HCDR3 comprising or consisting of the amino acid sequence of SEQ ID NO: 284; and a light chain variable region comprising: (i) a LCDR1 comprising or consisting of the amino acid sequence of SEQ ID NO: 285; (ii) a LCDR2 comprising or consisting of the amino acid sequence of SEQ ID NO: 286; and (iii) a LCDR3 comprising or consisting of the amino acid sequence of SEQ ID NO: 287, wherein the CDRs are defined according to the Kabat numbering system; and/or a heavy chain variable region comprising: (i) a HCDR1 comprising or consisting of the amino acid sequence of SEQ ID NO: 288; (ii) a HCDR2 comprising or consisting of the amino acid sequence of SEQ ID NO: 289, and (iii) a HCDR3 comprising or consisting of the amino acid sequence of SEQ ID NO: 290, and a light chain variable region comprising: (i) a LCDR1 comprising or consisting of the amino acid sequence of SEQ ID NO: 291; (ii) a LCDR2 comprising or consisting of the amino acid sequence of SEQ ID NO: 292; and (iii) a LCDR3 comprising or consisting of the amino acid sequence of SEQ ID NO: 293, wherein the CDRs are defined according to the IMGT numbering system.

In one embodiment, the antibody-drug conjugate comprises an anti-B7-H4 antibody or fragment thereof comprising a heavy chain variable region comprising or consisting of the amino acid sequence of SEQ ID NO: 294, or an amino acid sequence having at least 80%, 85%, 90%, 95%, 96%, 97%, 98%, or 99% identity thereto; and a light chain variable region comprising or consisting of the amino acid sequence of SEQ ID NO: 295 or an amino acid sequence having at least 80%, 85%, 90%, 95%, 96%, 97%, 98%, or 99% identity thereto.

In one embodiment, the antibody-drug conjugate comprises an anti-B7-H4 antibody or fragment thereof comprising a heavy chain comprising or consisting of the amino acid sequence of the amino acid sequence of SEQ ID NO: 296, or an amino acid sequence having at least 80%, 85%, 90%, 95%, 96%, 97%, 98%, or 99% identity thereto; and a light chain comprising or consisting of the amino acid sequence of SEQ ID NO: 297 or an amino acid sequence having at least 80%, 85%, 90%, 95%, 96%, 97%, 98%, or 99% identity thereto.

In one embodiment, the antibody-drug conjugate comprises an anti-B7-H4 antibody or fragment thereof comprising a heavy chain comprising or consisting of the amino acid sequence of the amino acid sequence of SEQ ID NO: 296; and a light chain comprising or consisting of the amino acid sequence of SEQ ID NO: 297.

In one embodiment, the antibody-drug conjugate comprises Felmetatug or a fragment thereof.

In one embodiment, the antibody-drug conjugate comprises Felmetatug.

An antibody-drug conjugate of formula (VIIb) where q is 4 and Ab is Felmetatug is referred to herein as “Compound 10” and may also be known as Felmetatug vedotin. In one embodiment, the antibody-drug conjugate is Felmetatug vedotin.

### Example Anti-ITGB6 Antibody Drug Conjugates

In one embodiment, the antibody-drug conjugate is of the formula (VIIb):

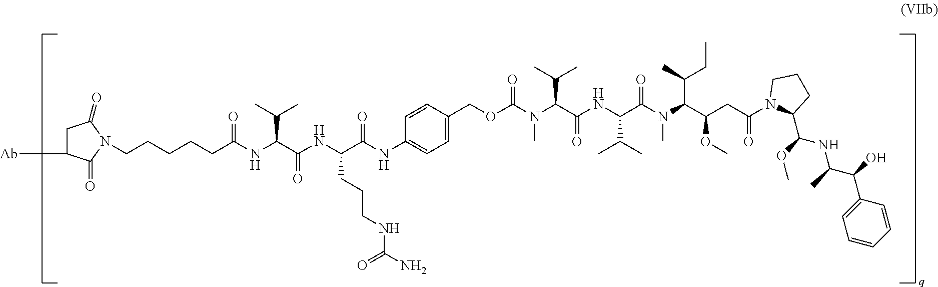

or a pharmaceutically acceptable salt thereof, wherein,
q represents a connection number, and q is selected from the group consisting of 1 to 16 preferably integers from 2 to 8; for example, q is an integer from 3 to 5, preferably 4, and
Ab is an anti-ITGB6 antibody or fragment thereof, as defined herein.

In one embodiment, the antibody-drug conjugate comprises an anti-ITGB6 antibody or fragment thereof comprising: a heavy chain variable region comprising: (i) a HCDR1 comprising or consisting of the amino acid sequence of SEQ ID NO: 298; (ii) a HCDR2 comprising or consisting of the amino acid sequence of SEQ ID NO: 299; and (iii) a HCDR3 comprising or consisting of the amino acid sequence of SEQ ID NO: 300; and a light chain variable region comprising: (i) a LCDR1 comprising or consisting of the amino acid sequence of SEQ ID NO: 301; (ii) a LCDR2 comprising or consisting of the amino acid sequence of SEQ ID NO: 302; and (iii) a LCDR3 comprising or consisting of the amino acid sequence of SEQ ID NO: 303, wherein the CDRs are defined according to the Kabat numbering system; and/or a heavy chain variable region comprising: (i) a HCDR1 comprising or consisting of the amino acid sequence of SEQ ID NO: 304; (ii) a HCDR2 comprising or consisting of the amino acid sequence of SEQ ID NO: 305; and (iii) a HCDR3 comprising or consisting of the amino acid sequence of SEQ ID NO: 306; and a light chain variable region comprising: (i) a LCDR1 comprising or consisting of the amino acid sequence of SEQ ID NO: 307; (ii) a LCDR2 comprising or consisting of the amino acid sequence of SEQ ID NO: 308; and (iii) a LCDR3 comprising or consisting of the amino acid sequence of SEQ ID NO: 309, wherein the CDRs are defined according to the IMGT numbering system.

In one embodiment, the antibody-drug conjugate comprises an anti-ITGB6 antibody or fragment thereof comprising a heavy chain variable region comprising or consisting of the amino acid sequence of SEQ ID NO: 310, or an amino acid sequence having at least 80%, 85%, 90%, 95%, 96%, 97%, 98%, or 99% identity thereto; and a light chain variable region comprising or consisting of the amino acid sequence of SEQ ID NO: 311 or an amino acid sequence having at least 80%, 85%, 90%, 95%, 96%, 97%, 98%, or 99% identity thereto.

In one embodiment, the antibody-drug conjugate comprises an anti-ITGB6 antibody or fragment thereof comprising a heavy chain comprising or consisting of the amino acid sequence of the amino acid sequence of SEQ ID NO: 312, or an amino acid sequence having at least 80%, 85%, 90%, 95%, 96%, 97%, 98%, or 99% identity thereto; and a light chain comprising or consisting of the amino acid sequence of SEQ ID NO: 313 or an amino acid sequence having at least 80%, 85%, 90%, 95%, 96%, 97%, 98%, or 99% identity thereto.

In one embodiment, the antibody-drug conjugate comprises an anti-ITGB6 antibody or fragment thereof comprising a heavy chain comprising or consisting of the amino acid sequence of the amino acid sequence of SEQ ID NO: 312; and a light chain comprising or consisting of the amino acid sequence of SEQ ID NO: 313.

In one embodiment, the antibody-drug conjugate comprises Sigvotatug or a fragment thereof.

In one embodiment, the antibody-drug conjugate comprises Sigvotatug.

An antibody-drug conjugate of formula (VIIb) where q is 4 and Ab is Sigvotatug is referred to herein as “Compound 11” and may also be known as Sigvotatug vedotin. In one embodiment, the antibody-drug conjugate is Sigvotatug vedotin.

### Example Anti-HER2 Antibody Drug Conjugates

In one embodiment, the antibody-drug conjugate is of the formula (VIIb):

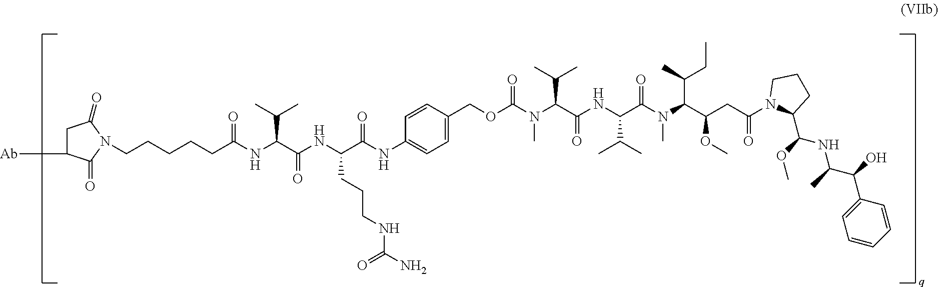

or a pharmaceutically acceptable salt thereof, wherein,
q represents a connection number, and q is selected from the group consisting of 1 to 16 preferably integers from 2 to 8; for example, q is an integer from 3 to 5, preferably 4, and
Ab is an anti-HER2 antibody or fragment thereof, as defined herein.

In one embodiment, the antibody-drug conjugate comprises an anti-HER2 antibody or fragment thereof comprising: a heavy chain variable region comprising: (i) a HCDR1 comprising or consisting of the amino acid sequence of SEQ ID NO: 314; (ii) a HCDR2 comprising or consisting of the amino acid sequence of SEQ ID NO: 315, and (iii) a HCDR3 comprising or consisting of the amino acid sequence of SEQ ID NO 316, and a light chain variable region comprising: (i) a LCDR1 comprising or consisting of the amino acid sequence of SEQ ID NO: 317; (ii) a LCDR2 comprising or consisting of the amino acid sequence of SEQ ID NO: 318; and (iii) a LCDR3 comprising or consisting of the amino acid sequence of SEQ ID NO: 319, wherein the CDRs are defied according to the Kabat numbering system; and/or a heavy chain variable region comprising: (i) a HCDR1 comprising or consisting of the amino acid sequence of SEQ ID NO: 320; (ii) a HCDR2 comprising or consisting of the amino acid sequence of SEQ ID NO: 321 and (iii) a HCDR3 comprising or consisting of the amino acid sequence of SEQ ID NO: 322 and a light chain variable region comprising: (i) a LCDR1 comprising or consisting of the amino acid sequence of SEQ ID NO: 323; (ii) a LCDR2 comprising or consisting of the amino acid sequence of SEQ ID NO: 324; and (iii) a LCDR3 comprising or consisting of the amino acid sequence of SEQ ID NO: 325, wherein the CDRs are defined according to the IMGT numbering system.

In one embodiment, the antibody-drug conjugate comprises an anti-HER2 antibody or fragment thereof comprising a heavy chain variable region comprising or consisting of the amino acid sequence of SEQ ID NO: 326, or an amino acid sequence having at least 80%, 85%, 90%, 95%, 96%, 97%, 98%, or 99% identity thereto; and a light chain variable region comprising or consisting of the amino acid sequence of SEQ ID NO: 327 or an amino acid sequence having at least 80%, 85%, 90%, 95%, 96%, 97%, 98%, or 99% identity thereto.

In one embodiment, the antibody-drug conjugate comprises an anti-HER2 antibody or fragment thereof comprising a heavy chain comprising or consisting of the amino acid sequence of the amino acid sequence of SEQ ID NO: 328, or an amino acid sequence having at least 80%, 85%, 90%, 95%, 96%, 97%, 98%, or 99% identity thereto; and a light chain comprising or consisting of the amino acid sequence of SEQ ID NO: 329 or an amino acid sequence having at least 80%, 85%, 90%, 95%, 96%, 97%, 98%, or 99% identity thereto.

In one embodiment, the antibody-drug conjugate comprises an anti-HER2 antibody or fragment thereof comprising a heavy chain comprising or consisting of the amino acid sequence of the amino acid sequence of SEQ ID NO: 328; and a light chain comprising or consisting of the amino acid sequence of SEQ ID NO: 329.

In one embodiment, the antibody-drug conjugate comprises Disitamab or a fragment thereof.

In one embodiment, the antibody-drug conjugate comprises Disitamab.

An antibody-drug conjugate of formula (VIIb) where q is 4 and Ab is Disitamab is referred to herein as “Compound 12” and may also be known as Disitamab vedotin. In one embodiment, the antibody-drug conjugate is Disitamab vedotin.

### The Binding Agent

Without wishing to be bound by theory, the combination of ADCs and the binding agent (e.g. an anti PD-L1 anti-VEGF bispecific antibody) beneficially, e.g. synergistically, increases the treatment efficacy in a variety of solid tumour types such as lung cancer, cervical cancer, ovarian cancer, without inducing relevant overlapping toxicity.

The binding agent component of the combination of the invention is a binding agent comprising (i) a first binding region which binds to PD-1, PD-L1, or both and which antagonizes the PD-1/PD-L1 interaction; and (ii) a second binding region which binds to VEGF, VEGFR, or both and which antagonizes the VEGF/VEGFR interaction (this binding agent is also referred to herein as “anti-PD-1/PD-L1xVEGF/VEGFR binding agent”). Upon administration, the anti-PD-1/PD-L1xVEGF/VEGFR binding agent binds to both (i) PD-1/PD-L1 and (ii) VEGF/VEGFR and therefore blocks or partially reduces the PD-1/PD-L1 and VEGF/VEGFR signaling pathways. This results in a decreased immunosuppression in the tumor microenvironment and reduced neo-angiogenesis. Thus, the co-administration of an anti-PD-1/PD-L1xVEGF/VEGFR binding agent as described herein can also reduce immunosuppression in the tumor microenvironment and/or reduce neo-angiogenesis and, thus, can enhance the tumor-antigen specific immune response of the ADC, thereby resulting in an increased effectiveness of the treatment of tumors.

The second binding region of the binding agent (e.g. the antiangiogenic part of a bispecific antibody) is contemplated to facilitate ADC penetration and exposure of tumour cells, allowing for ADC dose reduction and resulting in a lower ADC toxicity. For the immunotherapy component, it is suggested that the ADCs can increase the efficacy of immunotherapeutic agents. The implicated mode of action of this combination strategy includes induction of immunogenic cell death, dendritic cell maturation, increase of T-cell infiltration, as well as potentiation of immunological memory and expression of immune-regulatory proteins such as PD-L1 and MHC.

The binding agent may be formulated for administration to a subject. The binding agent may be formulated as a liquid, a solid, or a combination thereof. In some embodiments, the binding agent is formulated as a liquid. The binding agent may be formulated for injection or infusion. In some embodiments, the binding agent is formulated for intravenous administration.

First Binding Region which Binds to PD-1, PD-L1, or Both and which Antagonizes the PD-1/PD-L1 Interaction

One component of the anti-PD-1/PD-L1xVEGF/VEGFR binding agent is a first binding region which binds to PD-1, PD-L1, or both and which antagonizes the PD-1/PD-L1 interaction. Thus, this first binding region comprises an antagonist of the PD-1/PD-L1 interaction, such as an anti-PD-1 antibody, a PD-1 binding fragment thereof, an anti-PD-L1 antibody, or a PD-L1 binding fragment thereof.

### A1. Anti-PD-1 Antibodies or PD-1 Binding Fragments Thereof

In some embodiments, the first binding region which binds to PD-1, PD-L1, or both and which antagonizes the PD-1/PD-L1 interaction comprises an anti-PD-1 antibody or a PD-1 binding fragment thereof.

In some embodiments, PD-1 is human PD-1, in particular human PD-1 comprising the sequence set forth in SEQ ID NO: 169, 170, or 171.

In some embodiments, the anti-PD-1 antibody or a PD-1 binding fragment thereof (anti-PD-1 antibody or fragment thereof) specifically binds to PD-1.

In some embodiments, the anti-PD-1 antibody or fragment thereof is selected from the group consisting of pembrolizumab, nivolumab, cemiplimab, dostarlimab, toripalimab, retifanlimab, and PD-1 binding fragments thereof.

In some embodiments, the anti-PD-1 antibody or fragment thereof is a monoclonal antibody or a fragment thereof.

In some embodiments, the anti-PD-1 antibody or fragment thereof is a humanized antibody. In some embodiments, the anti-PD-1 antibody or fragment thereof is a monoclonal humanized antibody. In some embodiments, the fragment of the anti-PD-1 antibody may be any antigen-binding fragment of the anti-PD-1 antibody, for example a Fab, a Fab′, a F(ab′)2, a Fv, a scFv, a Fab′-SH, an sdAb, or a VHH. In some embodiments, the anti-PD-1 antibody or fragment thereof is selected from a full-length anti-PD-1 antibody, an scFv of an anti-PD-1 antibody or a VHH of an anti-PD-1 antibody. In some embodiments, the anti-PD-1 antibody or fragment thereof is an scFv of an anti-PD-1 antibody. In some embodiments, the anti-PD-1 antibody or fragment thereof is a VHH of an anti-PD-1 antibody.

In some embodiments, the anti-PD-1 antibody or fragment thereof may comprise a variable region that specifically binds to PD-1. In some embodiments, the anti-PD-1 antibody or fragment thereof comprises a heavy chain variable region and/or a light chain variable domain. In some embodiments, the anti-PD-1 antibody or fragment thereof comprises a heavy chain variable region and a light chain variable domain. In some embodiments, the anti-PD-1 antibody or fragment thereof comprises a constant region, preferably derived from a human antibody, preferably the constant region is selected from the constant region of human IgG1, IgG2, IgG3 or IgG4. In some embodiments, the anti-PD-1 antibody or fragment thereof comprises a heavy chain and/or a light chain. In some embodiments, the anti-PD-1 antibody or fragment thereof comprises a heavy chain and a light chain. In some embodiments, the anti-PD-1 antibody or fragment thereof comprises or consists of a heavy chain and a light chain as a single chain (scFv).

Exemplary anti-PD-1 antibody CDRs, variable region sequences, and heavy and light chain sequences are provided in Table 1, e.g., for anti-PD-1 antibodies A1 and A2, respectively.

In some embodiments, the anti-PD-1 antibody or fragment thereof is defined by one or more sequences of the anti-PD-1 antibody A1 shown in Table 1.

In some embodiments, the CDRs are defined according to the IMGT numbering system.

Thus, in some embodiments, (I) a VH of the anti-PD-1 antibody or fragment thereof comprises: (i) a CDR1 comprising or consisting of the amino acid sequence of SEQ ID NO: 178, or a variant thereof having up to three amino acid substitutions, additions or deletions;
(ii) a CDR2 comprising or consisting of the amino acid sequence of SEQ ID NO: 179, or a variant thereof having up to three amino acid substitutions, additions or deletions; and (iii) a CDR3 comprising or consisting of the amino acid sequence of SEQ ID NO: 180, or a variant thereof having up to three amino acid substitutions, additions or deletions, and/or (II) a VL of the anti-PD-1 antibody or fragment thereof comprises: (i) a CDR1 comprising or consisting of the amino acid sequence of SEQ ID NO: 181, or a variant thereof having up to three amino acid substitutions, additions or deletions; (ii) a CDR2 comprising or consisting of the amino acid sequence of SEQ ID NO: 182, or a variant thereof having up to three amino acid substitutions, additions or deletions; and (iii) a CDR3 comprising or consisting of the amino acid sequence of SEQ ID NO: 183, or a variant thereof having up to three amino acid substitutions, additions or deletions, wherein the CDRs are defined according to the IMGT numbering system. In some embodiments, the CDR variants have up to two amino acid substitutions, additions or deletions. In some embodiments, the CDR variants have up to one amino acid substitution, addition or deletion. In some embodiments, the CDR variants have up to three amino acid substitutions. In some embodiments, the CDR variants have up to two amino acid substitutions. In some embodiments, the CDR variants have up to one amino acid substitution.

In some embodiments, the CDRs are defined according to the Kabat numbering system. Thus, in some embodiments, (I) a VH of the anti-PD-1 antibody or fragment thereof comprises: (i) a CDR1 comprising or consisting of the amino acid sequence of SEQ ID NO: 184, or a variant thereof having up to three amino acid substitutions, additions or deletions; (ii) a CDR2 comprising or consisting of the amino acid sequence of SEQ ID NO: 185, or a variant thereof having up to three amino acid substitutions, additions or deletions; and (iii) a CDR3 comprising or consisting of the amino acid sequence of SEQ ID NO: 186, or a variant thereof having up to three amino acid substitutions, additions or deletions, and/or (II) a VL of the anti-PD-1 antibody or fragment thereof comprises: (i) a CDR1 comprising or consisting of the amino acid sequence of SEQ ID NO: 187, or a variant thereof having up to three amino acid substitutions, additions or deletions; (ii) a CDR2 comprising or consisting of the amino acid sequence of SEQ ID NO: 188, or a variant thereof having up to three amino acid substitutions, additions or deletions; and (iii) a CDR3 comprising or consisting of the amino acid sequence of SEQ ID NO: 183, or a variant thereof having up to three amino acid substitutions, additions or deletions, wherein the CDRs are defined according to the Kabat numbering system. In some embodiments, the CDR variants have up to two amino acid substitutions, additions or deletions. In some embodiments, the CDR variants have up to one amino acid substitution, addition or deletion. In some embodiments, the CDR variants have up to three amino acid substitutions. In some embodiments, the CDR variants have up to two amino acid substitutions. In some embodiments, the CDR variants have up to one amino acid substitution.

In some embodiments, the anti-PD-1 antibody or fragment thereof comprises or consists of an amino acid sequence having at least 80%, at least 85%, at least 90%, at least 95%, at least 96%, at least 97%, at least 98%, at least 99%, or 100% sequence identity to the amino acid sequence of SEQ ID NO: 189, and/or one or more (e.g., 1, 2, 3, 4, 5, 6, 7, 8, 9, or 10) amino acid additions, deletions and/or substitutions in a framework region thereof. In some embodiments, the anti-PD-1 antibody or fragment thereof comprises or consists of an amino acid sequence having at least 80%, at least 85%, at least 90%, at least 95%, at least 96%, at least 97%, at least 98%, at least 99%, or 100% sequence identity to the amino acid sequence of SEQ ID NO: 189, and/or one or more (e.g., 1, 2, 3, 4, 5, 6, 7, 8, 9, or 10) amino acid additions, deletions and/or substitutions in a framework region thereof, wherein the amino acid sequence comprises (i) the HCDR1, HCDR2, HCDR3, LCDR1, LCDR2, and LCDR3 sequences set forth in: SEQ ID NO: 178, 179, 180, 181, 182, and 183, respectively, or (ii) the HCDR1, HCDR2, HCDR3, LCDR1, LCDR2, and LCDR3 sequences set forth in: SEQ ID NO: 184, 185, 186, 187, 188, and 183, respectively. In some embodiments, the anti-PD-1 antibody or fragment thereof comprises or consists of an amino acid sequence having at least 80%, at least 85%, at least 90%, at least 95%, at least 96%, at least 97%, at least 98%, at least 99%, or 100% sequence identity to the amino acid sequence of SEQ ID NO: 189, wherein the amino acid sequence comprises (i) the HCDR1, HCDR2, HCDR3, LCDR1, LCDR2, and LCDR3 sequences set forth in: SEQ ID NO: 178, 179, 180, 181, 182, and 183, respectively, or (ii) the HCDR1, HCDR2, HCDR3, LCDR1, LCDR2, and LCDR3 sequences set forth in: SEQ ID NO: 184, 185, 186, 187, 188, and 183, respectively. In some embodiments, the anti-PD-1 antibody or fragment thereof comprises or consists of the amino acid sequence of SEQ ID NO: 189.

In some embodiments, the anti-PD-1 antibody or fragment thereof is an scFv comprising:
(i-1a) a heavy chain variable region (VH) comprising the HCDR1, HCDR2, and HCDR3 sequences set forth in: SEQ ID NO: 178, 179, and 180, respectively, and a light chain variable region (VL) comprising the LCDR1, LCDR2, and LCDR3 sequences set forth in: SEQ ID NO: 181, 182, and 183, respectively;
(i-1b) a heavy chain variable region (VH) comprising the HCDR1, HCDR2, and HCDR3 sequences set forth in: SEQ ID NO: 184, 185, and 186, respectively, and a light chain variable region (VL) comprising the LCDR1, LCDR2, and LCDR3 sequences set forth in: SEQ ID NO: 187, 188, and 183, respectively;
(i-1c) the amino acid sequence of SEQ ID NO: 189 or an amino acid sequence having at least 80%, at least 85%, at least 90%, at least 95%, at least 96%, at least 97%, at least 98%, at least 99%, or 100% sequence identity to the amino acid sequence SEQ ID NO: 189;
(i-1d) an amino acid sequence having at least 80%, at least 85%, at least 90%, at least 95%, at least 96%, at least 97%, at least 98%, at least 99%, or 100% sequence identity to the amino acid sequence SEQ ID NO: 189, and comprising the HCDR1, HCDR2, HCDR3, LCDR1, LCDR2, and LCDR3 sequences set forth in: SEQ ID NO: 178, 179, 180, 181, 182, and 183, respectively; or
(i-1e) an amino acid sequence having at least 80%, at least 85%, at least 90%, at least 95%, at least 96%, at least 97%, at least 98%, at least 99%, or 100% sequence identity to the amino acid sequence SEQ ID NO: 189, and comprising the HCDR1, HCDR2, HCDR3, LCDR1, LCDR2, and LCDR3 sequences set forth in: SEQ ID NO: 184, 185, 186, 187, 188, and 183, respectively.

In some alternative embodiments, the anti-PD-1 antibody or fragment thereof is defined by one or more sequences of the anti-PD-1 antibody A2 shown in Table 1.

In some embodiments, the CDRs are defined according to the Kabat numbering system. Thus, in some embodiments, the anti-PD-1 antibody or fragment thereof comprises: (i) a HCDR1 comprising or consisting of the amino acid sequence of SEQ ID NO: 110, or a variant thereof having up to three amino acid substitutions, additions or deletions; (ii) a HCDR2 comprising or consisting of the amino acid sequence of SEQ ID NO: 111, or a variant thereof having up to three amino acid substitutions, additions or deletions; and (iii) a HCDR3 comprising or consisting of the amino acid sequence of SEQ ID NO: 112 or 113, or a variant thereof having up to three amino acid substitutions, additions or deletions, wherein the CDRs are defined according to the Kabat numbering system. In some embodiments, the CDR variants have up to two amino acid substitutions, additions or deletions. In some embodiments, the CDR variants have up to one amino acid substitution, addition or deletion. In some embodiments, the CDR variants have up to three amino acid substitutions. In some embodiments, the CDR variants have up to two amino acid substitutions. In some embodiments, the CDR variants have up to one amino acid substitution.

In some embodiments, the CDRs are defined according to the IMGT numbering system. Thus, in some embodiments, the anti-PD-1 antibody or fragment thereof comprises: (i) a HCDR1 comprising or consisting of the amino acid sequence of SEQ ID NO: 114, or a variant thereof having up to three amino acid substitutions, additions or deletions; (ii) a HCDR2 comprising or consisting of the amino acid sequence of SEQ ID NO: 115, or a variant thereof having up to three amino acid substitutions, additions or deletions; and (iii) a HCDR3 comprising or consisting of the amino acid sequence of SEQ ID NO: 116 or 117, or a variant thereof having up to three amino acid substitutions, additions or deletions, wherein the CDRs are defined according to the IMGT numbering system. In some embodiments, the CDR variants have up to two amino acid substitutions, additions or deletions. In some embodiments, the CDR variants have up to one amino acid substitution, addition or deletion. In some embodiments, the CDR variants have up to three amino acid substitutions. In some embodiments, the CDR variants have up to two amino acid substitutions. In some embodiments, the CDR variants have up to one amino acid substitution.

In some embodiments, the anti-PD-1 antibody or fragment thereof comprises or consists of an amino acid sequence having at least 80%, at least 85%, at least 90%, at least 95%, at least 96%, at least 97%, at least 98%, at least 99%, or 100% sequence identity to the amino acid sequence of SEQ ID NO: 118, and/or one or more (e.g., 1, 2, 3, 4, 5, 6, 7, 8, 9, or 10) amino acid additions, deletions and/or substitutions in a framework region thereof. In some embodiments, the anti-PD-1 antibody or fragment thereof comprises or consists of an amino acid sequence having at least 80%, at least 85%, at least 90%, at least 95%, at least 96%, at least 97%, at least 98%, at least 99%, or 100% sequence identity to the amino acid sequence of SEQ ID NO: 118, and/or one or more (e.g., 1, 2, 3, 4, 5, 6, 7, 8, 9, or 10) amino acid additions, deletions and/or substitutions in a framework region thereof, wherein the amino acid sequence comprises (i) the HCDR1, HCDR2, and HCDR3 sequences set forth in: SEQ ID NO: 110, 111, and 112, respectively, or (ii) the HCDR1, HCDR2, and HCDR3 sequences set forth in: SEQ ID NO: 114, 115, and 116, respectively. In some embodiments, the anti-PD-1 antibody or fragment thereof comprises or consists of an amino acid sequence having at least 80%, at least 85%, at least 90%, at least 95%, at least 96%, at least 97%, at least 98%, at least 99%, or 100% sequence identity to the amino acid sequence of SEQ ID NO: 118, wherein the amino acid sequence comprises (i) the HCDR1, HCDR2, and HCDR3 sequences set forth in: SEQ ID NO: 110, 111, and 112, respectively, or (ii) the HCDR1, HCDR2, and HCDR3 sequences set forth in: SEQ ID NO: 114, 115, and 116, respectively. In some embodiments, the anti-PD-1 antibody or fragment thereof comprises or consists of the amino acid sequence of SEQ ID NO: 118.

In some embodiments, the anti-PD-1 antibody or fragment thereof comprises or consists of an amino acid sequence having at least 80%, at least 85%, at least 90%, at least 95%, at least 96%, at least 97%, at least 98%, at least 99%, or 100% sequence identity to the amino acid sequence of SEQ ID NO: 119, and/or one or more (e.g., 1, 2, 3, 4, 5, 6, 7, 8, 9, or 10) amino acid additions, deletions and/or substitutions in a framework region thereof. In some embodiments, the anti-PD-1 antibody or fragment thereof comprises or consists of an amino acid sequence having at least 80%, at least 85%, at least 90%, at least 95%, at least 96%, at least 97%, at least 98%, at least 99%, or 100% sequence identity to the amino acid sequence of SEQ ID NO: 119, and/or one or more (e.g., 1, 2, 3, 4, 5, 6, 7, 8, 9, or 10) amino acid additions, deletions and/or substitutions in a framework region thereof, wherein the amino acid sequence comprises (i) the HCDR1, HCDR2, and HCDR3 sequences set forth in: SEQ ID NO: 110, 111, and 113, respectively, or (ii) the HCDR1, HCDR2, and HCDR3 sequences set forth in: SEQ ID NO: 114, 115, and 117, respectively. In some embodiments, the anti-PD-1 antibody or fragment thereof comprises or consists of an amino acid sequence having at least 80%, at least 85%, at least 90%, at least 95%, at least 96%, at least 97%, at least 98%, at least 99%, or 100% sequence identity to the amino acid sequence of SEQ ID NO: 119, wherein the amino acid sequence comprises (i) the HCDR1, HCDR2, and HCDR3 sequences set forth in: SEQ ID NO: 110, 111, and 113, respectively, or (ii) the HCDR1, HCDR2, and HCDR3 sequences set forth in: SEQ ID NO: 114, 115, and 117, respectively. In some embodiments, the anti-PD-1 antibody or fragment thereof comprises or consists of the amino acid sequence of SEQ ID NO: 119.

Thus, in some embodiments, the anti-PD-1 antibody or fragment thereof is a VHH comprising:
(i-2a) the HCDR1, HCDR2, and HCDR3 sequences set forth in: SEQ ID NO: 110, 111, and 112, respectively, or a VH comprising the HCDR1, HCDR2, and HCDR3 sequences set forth in: SEQ ID NO: 110, 111, and 113, respectively;
(i-2b) the HCDR1, HCDR2, and HCDR3 sequences set forth in: SEQ ID NO: 114, 115, and 116, respectively, or a VH comprising the HCDR1, HCDR2, and HCDR3 sequences set forth in: SEQ ID NO: 114, 115, and 117, respectively;
(i-2c) the amino acid sequence of SEQ ID NO: 118 or an amino acid sequence having at least 80%, at least 85%, at least 90%, at least 95%, at least 96%, at least 97%, at least 98%, at least 99%, or 100% sequence identity to the amino acid sequence SEQ ID NO: 118, wherein the amino acid sequence having at least 80%, at least 85%, at least 90%, at least 95%, at least 96%, at least 97%, at least 98%, at least 99%, or 100% sequence identity to the amino acid sequence SEQ ID NO: 118 preferably comprises the HCDR1, HCDR2, and HCDR3 sequences set forth in: SEQ ID NO: 110, 111, and 112, respectively, or comprises the HCDR1, HCDR2, and HCDR3 sequences set forth in: SEQ ID NO: 114, 115, and 116, respectively; or
(i-2d) the amino acid sequence of SEQ ID NO: 119 or an amino acid sequence having at least 80%, at least 85%, at least 90%, at least 95%, at least 96%, at least 97%, at least 98%, at least 99%, or 100% sequence identity to the amino acid sequence SEQ ID NO: 119, wherein the amino acid sequence having at least 80%, at least 85%, at least 90%, at least 95%, at least 96%, at least 97%, at least 98%, at least 99%, or 100% sequence identity to the amino acid sequence SEQ ID NO: 119 preferably comprises the HCDR1, HCDR2, and HCDR3 sequences set forth in: SEQ ID NO: 110, 111, and 113, respectively, or comprises the HCDR1, HCDR2, and HCDR3 sequences set forth in: SEQ ID NO: 114, 115, and 117, respectively.

In some embodiments, the anti-PD-1 antibody or fragment thereof is defined by one or more sequences of the anti-PD-1 antibody A5, A6, A7 or A8 shown in Table 1.

In some embodiments, (I) a VH of the anti-PD-1 antibody or fragment thereof comprises: (i) a HCDR1 comprising or consisting of the amino acid sequence of SEQ ID NO: 330, or a variant thereof having up to three amino acid substitutions, additions or deletions; (ii) a HCDR2 comprising or consisting of the amino acid sequence of SEQ ID NO: 331, or a variant thereof having up to three amino acid substitutions, additions or deletions; and (iii) a HCDR3 comprising or consisting of the amino acid sequence of SEQ ID NO: 332, or a variant thereof having up to three amino acid substitutions, additions or deletions, and/or (II) a VL of the anti-PD-1 antibody or fragment thereof comprises: (i) a LCDR1 comprising or consisting of the amino acid sequence of SEQ ID NO: 333, or a variant thereof having up to three amino acid substitutions, additions or deletions; (ii) a LCDR2 comprising or consisting of the amino acid sequence of SEQ ID NO: 334, or a variant thereof having up to three amino acid substitutions, additions or deletions, (ii′) a LCDR2 comprising or consisting of the amino acid sequence of SEQ ID NO: 338, or a variant thereof having up to three amino acid substitutions, additions or deletions, (ii″) a LCDR2 comprising or consisting of the amino acid sequence of SEQ ID NO: 340, or a variant thereof having up to three amino acid substitutions, additions or deletions, or (ii′″) a LCDR2 comprising or consisting of the amino acid sequence of SEQ ID NO: 342, or a variant thereof having up to three amino acid substitutions, additions or deletions; and (iii) a LCDR3 comprising or consisting of the amino acid sequence of SEQ ID NO: 335, or a variant thereof having up to three amino acid substitutions, additions or deletions. In some embodiments, the CDR variants have up to two amino acid substitutions, additions or deletions. In some embodiments, the CDR variants have up to one amino acid substitution, addition or deletion. In some embodiments, the CDR variants have up to three amino acid substitutions. In some embodiments, the CDR variants have up to two amino acid substitutions. In some embodiments, the CDR variants have up to one amino acid substitution.

In some embodiments, (I) a VH of the anti-PD-1 antibody or fragment thereof comprises: (i) a HCDR1 comprising or consisting of the amino acid sequence of SEQ ID NO: 330, or a variant thereof having up to three amino acid substitutions, additions or deletions; (ii) a HCDR2 comprising or consisting of the amino acid sequence of SEQ ID NO: 331, or a variant thereof having up to three amino acid substitutions, additions or deletions; and (iii) a HCDR3 comprising or consisting of the amino acid sequence of SEQ ID NO: 332, or a variant thereof having up to three amino acid substitutions, additions or deletions, and/or (II) a VL of the anti-PD-1 antibody or fragment thereof comprises: (i) a LCDR1 comprising or consisting of the amino acid sequence of SEQ ID NO: 333, or a variant thereof having up to three amino acid substitutions, additions or deletions; (ii) a LCDR2 comprising or consisting of the amino acid sequence of SEQ ID NO: 334, or a variant thereof having up to three amino acid substitutions, additions or deletions; and (iii) a LCDR3 comprising or consisting of the amino acid sequence of SEQ ID NO: 335, or a variant thereof having up to three amino acid substitutions, additions or deletions. In some embodiments, the CDR variants have up to two amino acid substitutions, additions or deletions. In some embodiments, the CDR variants have up to one amino acid substitution, addition or deletion. In some embodiments, the CDR variants have up to three amino acid substitutions. In some embodiments, the CDR variants have up to two amino acid substitutions. In some embodiments, the CDR variants have up to one amino acid substitution.

In some embodiments, (I) a VH of the anti-PD-1 antibody or fragment thereof comprises: (i) a HCDR1 comprising or consisting of the amino acid sequence of SEQ ID NO: 330, or a variant thereof having up to three amino acid substitutions, additions or deletions; (ii) a HCDR2 comprising or consisting of the amino acid sequence of SEQ ID NO: 331, or a variant thereof having up to three amino acid substitutions, additions or deletions; and (iii) a HCDR3 comprising or consisting of the amino acid sequence of SEQ ID NO: 332, or a variant thereof having up to three amino acid substitutions, additions or deletions, and/or (II) a VL of the anti-PD-1 antibody or fragment thereof comprises: (i) a LCDR1 comprising or consisting of the amino acid sequence of SEQ ID NO: 333, or a variant thereof having up to three amino acid substitutions, additions or deletions; (ii) a LCDR2 comprising or consisting of the amino acid sequence of SEQ ID NO: 338, or a variant thereof having up to three amino acid substitutions, additions or deletions; and (iii) a LCDR3 comprising or consisting of the amino acid sequence of SEQ ID NO: 335, or a variant thereof having up to three amino acid substitutions, additions or deletions. In some embodiments, the CDR variants have up to two amino acid substitutions, additions or deletions. In some embodiments, the CDR variants have up to one amino acid substitution, addition or deletion. In some embodiments, the CDR variants have up to three amino acid substitutions. In some embodiments, the CDR variants have up to two amino acid substitutions. In some embodiments, the CDR variants have up to one amino acid substitution.

In some embodiments, (I) a VH of the anti-PD-1 antibody or fragment thereof comprises: (i) a HCDR1 comprising or consisting of the amino acid sequence of SEQ ID NO: 330, or a variant thereof having up to three amino acid substitutions, additions or deletions; (ii) a HCDR2 comprising or consisting of the amino acid sequence of SEQ ID NO: 331, or a variant thereof having up to three amino acid substitutions, additions or deletions; and (iii) a HCDR3 comprising or consisting of the amino acid sequence of SEQ ID NO: 332, or a variant thereof having up to three amino acid substitutions, additions or deletions, and/or (II) a VL of the anti-PD-1 antibody or fragment thereof comprises: (i) a LCDR1 comprising or consisting of the amino acid sequence of SEQ ID NO: 333, or a variant thereof having up to three amino acid substitutions, additions or deletions; (ii) a LCDR2 comprising or consisting of the amino acid sequence of SEQ ID NO: 340, or a variant thereof having up to three amino acid substitutions, additions or deletions; and (iii) a LCDR3 comprising or consisting of the amino acid sequence of SEQ ID NO: 335, or a variant thereof having up to three amino acid substitutions, additions or deletions. In some embodiments, the CDR variants have up to two amino acid substitutions, additions or deletions. In some embodiments, the CDR variants have up to one amino acid substitution, addition or deletion. In some embodiments, the CDR variants have up to three amino acid substitutions. In some embodiments, the CDR variants have up to two amino acid substitutions. In some embodiments, the CDR variants have up to one amino acid substitution.

In some embodiments, (I) a VH of the anti-PD-1 antibody or fragment thereof comprises: (i) a HCDR1 comprising or consisting of the amino acid sequence of SEQ ID NO: 330, or a variant thereof having up to three amino acid substitutions, additions or deletions; (ii) a HCDR2 comprising or consisting of the amino acid sequence of SEQ ID NO: 331, or a variant thereof having up to three amino acid substitutions, additions or deletions; and (iii) a HCDR3 comprising or consisting of the amino acid sequence of SEQ ID NO: 332, or a variant thereof having up to three amino acid substitutions, additions or deletions, and/or (II) a VL of the anti-PD-1 antibody or fragment thereof comprises: (i) a LCDR1 comprising or consisting of the amino acid sequence of SEQ ID NO: 333, or a variant thereof having up to three amino acid substitutions, additions or deletions; (ii) a LCDR2 comprising or consisting of the amino acid sequence of SEQ ID NO: 342, or a variant thereof having up to three amino acid substitutions, additions or deletions; and (iii) a LCDR3 comprising or consisting of the amino acid sequence of SEQ ID NO: 335, or a variant thereof having up to three amino acid substitutions, additions or deletions. In some embodiments, the CDR variants have up to two amino acid substitutions, additions or deletions. In some embodiments, the CDR variants have up to one amino acid substitution, addition or deletion. In some embodiments, the CDR variants have up to three amino acid substitutions. In some embodiments, the CDR variants have up to two amino acid substitutions. In some embodiments, the CDR variants have up to one amino acid substitution.

In some embodiments, the anti-PD-1 antibody or fragment thereof comprises a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 336, and/or one or more (e.g., 1, 2, 3, 4, 5, 6, 7, 8, 9, or 10) amino acid additions, deletions and/or substitutions in a framework region thereof. In some embodiments, the anti-PD-1 antibody or fragment thereof comprises a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 336, and/or one or more (e.g., 1, 2, 3, 4, 5, 6, 7, 8, 9, or 10) amino acid additions, deletions and/or substitutions in a framework region thereof, wherein the VH comprises the CDR1, CDR2, and CDR3 sequences set forth in: SEQ ID NO: 330, 331, and 332, respectively. In some embodiments, the anti-PD-1 antibody or fragment thereof comprises a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 336. In some embodiments, the anti-PD-1 antibody or fragment thereof comprises a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 336, wherein the VH comprises the CDR1, CDR2, and CDR3 sequences set forth in: SEQ ID NO: 330, 331, and 332, respectively. In some embodiments, the anti-PD-1 antibody or fragment thereof comprises a VH comprising or consisting of the amino acid sequence of SEQ ID NO: 336.

In some embodiments, the anti-PD-1 antibody or fragment thereof comprises a VL comprising or consisting of an amino acid sequence having at least 80%, at least 85%, at least 90%, at least 95%, at least 96%, at least 97%, at least 98%, at least 99%, or 100% sequence identity to the amino acid sequence SEQ ID NO: 337 and/or one or more (e.g., 1, 2, 3, 4, 5, 6, 7, 8, 9, or 10) amino acid additions, deletions and/or substitutions in a framework region thereof. In some embodiments, the anti-PD-1 antibody or fragment thereof comprises a VL comprising or consisting of an amino acid sequence having at least 80%, at least 85%, at least 90%, at least 95%, at least 96%, at least 97%, at least 98%, at least 99%, or 100% sequence identity to the amino acid sequence SEQ ID NO: 337 and/or one or more (e.g., 1, 2, 3, 4, 5, 6, 7, 8, 9, or 10) amino acid additions, deletions and/or substitutions in a framework region thereof, wherein the VL comprises the CDR1, CDR2, and CDR3 sequences set forth in: SEQ ID NO: 333, 334, and 335, respectively. In some embodiments, the anti-PD-1 antibody or fragment thereof comprises a VL comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 337. In some embodiments, the anti-PD-1 antibody or fragment thereof comprises a VL comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 337, wherein the VL comprises the CDR1, CDR2, and CDR3 sequences set forth in: SEQ ID NO: 333, 334, and 335, respectively. In some embodiments, the anti-PD-1 antibody or fragment thereof comprises a VL comprising or consisting of the amino acid sequence of SEQ ID NO: 337.

In some embodiments, the anti-PD-1 antibody or fragment thereof comprises a VL comprising or consisting of an amino acid sequence having at least 80%, at least 85%, at least 90%, at least 95%, at least 96%, at least 97%, at least 98%, at least 99%, or 100% sequence identity to the amino acid sequence SEQ ID NO: 339 and/or one or more (e.g., 1, 2, 3, 4, 5, 6, 7, 8, 9, or 10) amino acid additions, deletions and/or substitutions in a framework region thereof. In some embodiments, the anti-PD-1 antibody or fragment thereof comprises a VL comprising or consisting of an amino acid sequence having at least 80%, at least 85%, at least 90%, at least 95%, at least 96%, at least 97%, at least 98%, at least 99%, or 100% sequence identity to the amino acid sequence SEQ ID NO: 339 and/or one or more (e.g., 1, 2, 3, 4, 5, 6, 7, 8, 9, or 10) amino acid additions, deletions and/or substitutions in a framework region thereof, wherein the VL comprises the CDR1, CDR2, and CDR3 sequences set forth in: SEQ ID NO: 333, 338, and 335, respectively. In some embodiments, the anti-PD-1 antibody or fragment thereof comprises a VL comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 339. In some embodiments, the anti-PD-1 antibody or fragment thereof comprises a VL comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 339, wherein the VL comprises the CDR1, CDR2, and CDR3 sequences set forth in: SEQ ID NO: 333, 338, and 335, respectively. In some embodiments, the anti-PD-1 antibody or fragment thereof comprises a VL comprising or consisting of the amino acid sequence of SEQ ID NO: 339.

In some embodiments, the anti-PD-1 antibody or fragment thereof comprises a VL comprising or consisting of an amino acid sequence having at least 80%, at least 85%, at least 90%, at least 95%, at least 96%, at least 97%, at least 98%, at least 99%, or 100% sequence identity to the amino acid sequence SEQ ID NO: 341 and/or one or more (e.g., 1, 2, 3, 4, 5, 6, 7, 8, 9, or 10) amino acid additions, deletions and/or substitutions in a framework region thereof. In some embodiments, the anti-PD-1 antibody or fragment thereof comprises a VL comprising or consisting of an amino acid sequence having at least 80%, at least 85%, at least 90%, at least 95%, at least 96%, at least 97%, at least 98%, at least 99%, or 100% sequence identity to the amino acid sequence SEQ ID NO: 341 and/or one or more (e.g., 1, 2, 3, 4, 5, 6, 7, 8, 9, or 10) amino acid additions, deletions and/or substitutions in a framework region thereof, wherein the VL comprises the CDR1, CDR2, and CDR3 sequences set forth in: SEQ ID NO: 333, 340, and 335, respectively. In some embodiments, the anti-PD-1 antibody or fragment thereof comprises a VL comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 341. In some embodiments, the anti-PD-1 antibody or fragment thereof comprises a VL comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 341, wherein the VL comprises the CDR1, CDR2, and CDR3 sequences set forth in: SEQ ID NO: 333, 340, and 335, respectively. In some embodiments, the anti-PD-1 antibody or fragment thereof comprises a VL comprising or consisting of the amino acid sequence of SEQ ID NO: 341.

In some embodiments, the anti-PD-1 antibody or fragment thereof comprises a VL comprising or consisting of an amino acid sequence having at least 80%, at least 85%, at least 90%, at least 95%, at least 96%, at least 97%, at least 98%, at least 99%, or 100% sequence identity to the amino acid sequence SEQ ID NO: 343 and/or one or more (e.g., 1, 2, 3, 4, 5, 6, 7, 8, 9, or 10) amino acid additions, deletions and/or substitutions in a framework region thereof. In some embodiments, the anti-PD-1 antibody or fragment thereof comprises a VL comprising or consisting of an amino acid sequence having at least 80%, at least 85%, at least 90%, at least 95%, at least 96%, at least 97%, at least 98%, at least 99%, or 100% sequence identity to the amino acid sequence SEQ ID NO: 343 and/or one or more (e.g., 1, 2, 3, 4, 5, 6, 7, 8, 9, or 10) amino acid additions, deletions and/or substitutions in a framework region thereof, wherein the VL comprises the CDR1, CDR2, and CDR3 sequences set forth in: SEQ ID NO: 333, 342, and 335, respectively. In some embodiments, the anti-PD-1 antibody or fragment thereof comprises a VL comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 343. In some embodiments, the anti-PD-1 antibody or fragment thereof comprises a VL comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 343, wherein the VL comprises the CDR1, CDR2, and CDR3 sequences set forth in: SEQ ID NO: 333, 342, and 335, respectively. In some embodiments, the anti-PD-1 antibody or fragment thereof comprises a VL comprising or consisting of the amino acid sequence of SEQ ID NO: 343.

In some embodiments, the anti-PD-1 antibody or fragment thereof comprises (I) a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 336, and/or one or more (e.g., 1, 2, 3, 4, 5, 6, 7, 8, 9, or 10) amino acid additions, deletions and/or substitutions in a framework region thereof, and (II) a VL comprising or consisting of an amino acid sequence having at least 80%, at least 85%, at least 90%, at least 95%, at least 96%, at least 97%, at least 98%, at least 99%, or 100% sequence identity to the amino acid sequence SEQ ID NO: 337 and/or one or more (e.g., 1, 2, 3, 4, 5, 6, 7, 8, 9, or 10) amino acid additions, deletions and/or substitutions in a framework region thereof. In some embodiments, the anti-PD-1 antibody or fragment thereof comprises a (I) a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 336, and/or one or more (e.g., 1, 2, 3, 4, 5, 6, 7, 8, 9, or 10) amino acid additions, deletions and/or substitutions in a framework region thereof, and (II) a VL comprising or consisting of an amino acid sequence having at least 80%, at least 85%, at least 90%, at least 95%, at least 96%, at least 97%, at least 98%, at least 99%, or 100% sequence identity to the amino acid sequence SEQ ID NO: 337 and/or one or more (e.g., 1, 2, 3, 4, 5, 6, 7, 8, 9, or 10) amino acid additions, deletions and/or substitutions in a framework region thereof, wherein the VH comprises the CDR1, CDR2, and CDR3 sequences set forth in: SEQ ID NO: 330, 331, and 332, respectively, and the VL comprises the CDR1, CDR2, and CDR3 sequences set forth in: SEQ ID NO: 333, 334, and 335, respectively. In some embodiments, the anti-PD-1 antibody or fragment thereof comprises (I) a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 336, and (II) a VL comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 337. In some embodiments, the anti-PD-1 antibody or fragment thereof comprises (I) a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 336, and (II) a VL comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 337, wherein the VH comprises the CDR1, CDR2, and CDR3 sequences set forth in: SEQ ID NO: 330, 331, and 332, respectively, and the VL comprises the CDR1, CDR2, and CDR3 sequences set forth in: SEQ ID NO: 333, 334, and 335, respectively. In some embodiments, the anti-PD-1 antibody or fragment thereof comprises (I) a VH comprising or consisting of the amino acid sequence of SEQ ID NO: 336, and (II) comprises a VL comprising or consisting of the amino acid sequence of SEQ ID NO: 337.

In some embodiments, the anti-PD-1 antibody or fragment thereof comprises (I) a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 336, and/or one or more (e.g., 1, 2, 3, 4, 5, 6, 7, 8, 9, or 10) amino acid additions, deletions and/or substitutions in a framework region thereof, and (II) a VL comprising or consisting of an amino acid sequence having at least 80%, at least 85%, at least 90%, at least 95%, at least 96%, at least 97%, at least 98%, at least 99%, or 100% sequence identity to the amino acid sequence SEQ ID NO: 339 and/or one or more (e.g., 1, 2, 3, 4, 5, 6, 7, 8, 9, or 10) amino acid additions, deletions and/or substitutions in a framework region thereof. In some embodiments, the anti-PD-1 antibody or fragment thereof comprises a (I) a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 336, and/or one or more (e.g., 1, 2, 3, 4, 5, 6, 7, 8, 9, or 10) amino acid additions, deletions and/or substitutions in a framework region thereof, and (II) a VL comprising or consisting of an amino acid sequence having at least 80%, at least 85%, at least 90%, at least 95%, at least 96%, at least 97%, at least 98%, at least 99%, or 100% sequence identity to the amino acid sequence SEQ ID NO: 339 and/or one or more (e.g., 1, 2, 3, 4, 5, 6, 7, 8, 9, or 10) amino acid additions, deletions and/or substitutions in a framework region thereof, wherein the VH comprises the CDR1, CDR2, and CDR3 sequences set forth in: SEQ ID NO: 330, 331, and 332, respectively, and the VL comprises the CDR1, CDR2, and CDR3 sequences set forth in: SEQ ID NO: 333, 338, and 335, respectively. In some embodiments, the anti-PD-1 antibody or fragment thereof comprises (I) a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 336, and (II) a VL comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 339. In some embodiments, the anti-PD-1 antibody or fragment thereof comprises (I) a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 336, and (II) a VL comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 339, wherein the VH comprises the CDR1, CDR2, and CDR3 sequences set forth in: SEQ ID NO: 330, 331, and 332, respectively, and the VL comprises the CDR1, CDR2, and CDR3 sequences set forth in: SEQ ID NO: 333, 338, and 335, respectively. In some embodiments, the anti-PD-1 antibody or fragment thereof comprises (I) a VH comprising or consisting of the amino acid sequence of SEQ ID NO: 336, and (II) comprises a VL comprising or consisting of the amino acid sequence of SEQ ID NO: 339.

In some embodiments, the anti-PD-1 antibody or fragment thereof comprises (I) a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 336, and/or one or more (e.g., 1, 2, 3, 4, 5, 6, 7, 8, 9, or 10) amino acid additions, deletions and/or substitutions in a framework region thereof, and (II) a VL comprising or consisting of an amino acid sequence having at least 80%, at least 85%, at least 90%, at least 95%, at least 96%, at least 97%, at least 98%, at least 99%, or 100% sequence identity to the amino acid sequence SEQ ID NO: 341 and/or one or more (e.g., 1, 2, 3, 4, 5, 6, 7, 8, 9, or 10) amino acid additions, deletions and/or substitutions in a framework region thereof. In some embodiments, the anti-PD-1 antibody or fragment thereof comprises a (I) a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 336, and/or one or more (e.g., 1, 2, 3, 4, 5, 6, 7, 8, 9, or 10) amino acid additions, deletions and/or substitutions in a framework region thereof, and (II) a VL comprising or consisting of an amino acid sequence having at least 80%, at least 85%, at least 90%, at least 95%, at least 96%, at least 97%, at least 98%, at least 99%, or 100% sequence identity to the amino acid sequence SEQ ID NO: 341 and/or one or more (e.g., 1, 2, 3, 4, 5, 6, 7, 8, 9, or 10) amino acid additions, deletions and/or substitutions in a framework region thereof, wherein the VH comprises the CDR1, CDR2, and CDR3 sequences set forth in: SEQ ID NO: 330, 331, and 332, respectively, and the VL comprises the CDR1, CDR2, and CDR3 sequences set forth in: SEQ ID NO: 333, 340, and 335, respectively. In some embodiments, the anti-PD-1 antibody or fragment thereof comprises (I) a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 336, and (II) a VL comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 341. In some embodiments, the anti-PD-1 antibody or fragment thereof comprises (I) a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 336, and (II) a VL comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 341, wherein the VH comprises the CDR1, CDR2, and CDR3 sequences set forth in: SEQ ID NO: 330, 331, and 332, respectively, and the VL comprises the CDR1, CDR2, and CDR3 sequences set forth in: SEQ ID NO: 333, 340, and 335, respectively. In some embodiments, the anti-PD-1 antibody or fragment thereof comprises (I) a VH comprising or consisting of the amino acid sequence of SEQ ID NO: 336, and (II) comprises a VL comprising or consisting of the amino acid sequence of SEQ ID NO: 341.

In some embodiments, the anti-PD-1 antibody or fragment thereof comprises (I) a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 336, and/or one or more (e.g., 1, 2, 3, 4, 5, 6, 7, 8, 9, or 10) amino acid additions, deletions and/or substitutions in a framework region thereof, and (II) a VL comprising or consisting of an amino acid sequence having at least 80%, at least 85%, at least 90%, at least 95%, at least 96%, at least 97%, at least 98%, at least 99%, or 100% sequence identity to the amino acid sequence SEQ ID NO: 343 and/or one or more (e.g., 1, 2, 3, 4, 5, 6, 7, 8, 9, or 10) amino acid additions, deletions and/or substitutions in a framework region thereof. In some embodiments, the anti-PD-1 antibody or fragment thereof comprises a (I) a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 336, and/or one or more (e.g., 1, 2, 3, 4, 5, 6, 7, 8, 9, or 10) amino acid additions, deletions and/or substitutions in a framework region thereof, and (II) a VL comprising or consisting of an amino acid sequence having at least 80%, at least 85%, at least 90%, at least 95%, at least 96%, at least 97%, at least 98%, at least 99%, or 100% sequence identity to the amino acid sequence SEQ ID NO: 343 and/or one or more (e.g., 1, 2, 3, 4, 5, 6, 7, 8, 9, or 10) amino acid additions, deletions and/or substitutions in a framework region thereof, wherein the VH comprises the CDR1, CDR2, and CDR3 sequences set forth in: SEQ ID NO: 330, 331, and 332, respectively, and the VL comprises the CDR1, CDR2, and CDR3 sequences set forth in: SEQ ID NO: 333, 342, and 335, respectively. In some embodiments, the anti-PD-1 antibody or fragment thereof comprises (I) a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 336, and (II) a VL comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 343. In some embodiments, the anti-PD-1 antibody or fragment thereof comprises (I) a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 336, and (II) a VL comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 343, wherein the VH comprises the CDR1, CDR2, and CDR3 sequences set forth in: SEQ ID NO: 330, 331, and 332, respectively, and the VL comprises the CDR1, CDR2, and CDR3 sequences set forth in: SEQ ID NO: 333, 342, and 335, respectively. In some embodiments, the anti-PD-1 antibody or fragment thereof comprises (I) a VH comprising or consisting of the amino acid sequence of SEQ ID NO: 336, and (II) comprises a VL comprising or consisting of the amino acid sequence of SEQ ID NO: 343.

Thus, in some embodiments, the anti-PD-1 antibody or fragment thereof is a fragment of a humanized anti-PD-1 antibody comprising:
(i-4a) a heavy chain variable region (VH) comprising the HCDR1, HCDR2, and HCDR3 sequences of SEQ ID NO: 336, and a light chain variable region (VL) comprising the LCDR1, LCDR2, and LCDR3 sequences of SEQ ID NO: 337;
(i-4b) a heavy chain variable region (VH) comprising the HCDR1, HCDR2, and HCDR3 sequences set forth in: SEQ ID NO: 330, 331, and 332, respectively, and a light chain variable region (VL) comprising the LCDR1, LCDR2, and LCDR3 sequences set forth in: SEQ ID NO: 333, 334, and 335, respectively;
(i-4c) a heavy chain variable region (VH) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 336, and a light chain variable region (VL) comprising an amino acid sequence having at least 80%, at least 85%, at least 90%, at least 95%, at least 96%, at least 97%, at least 98%, at least 99%, or 100% sequence identity to the amino acid sequence SEQ ID NO: 337; or
(i-4d) a heavy chain variable region (VH) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 336, and a light chain variable region (VL) comprising an amino acid sequence having at least 80%, at least 85%, at least 90%, at least 95%, at least 96%, at least 97%, at least 98%, at least 99%, or 100% sequence identity to the amino acid sequence SEQ ID NO: 337, wherein the VH comprises the HCDR1, HCDR2, and HCDR3 sequences set forth in: SEQ ID NO: 330, 331, and 332, respectively, and the VL comprises the LCDR1, LCDR2, and LCDR3 sequences set forth in: SEQ ID NO: 333, 334, and 335, respectively.

Thus, in some embodiments, the anti-PD-1 antibody or fragment thereof is a fragment of a humanized anti-PD-1 antibody comprising:
(i-4a) a heavy chain variable region (VH) comprising the HCDR1, HCDR2, and HCDR3 sequences of SEQ ID NO: 336, and a light chain variable region (VL) comprising the LCDR1, LCDR2, and LCDR3 sequences of SEQ ID NO: 339;
(i-4b) a heavy chain variable region (VH) comprising the HCDR1, HCDR2, and HCDR3 sequences set forth in: SEQ ID NO: 330, 331, and 332, respectively, and a light chain variable region (VL) comprising the LCDR1, LCDR2, and LCDR3 sequences set forth in: SEQ ID NO: 333, 338, and 335, respectively;
(i-4c) a heavy chain variable region (VH) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 336, and a light chain variable region (VL) comprising an amino acid sequence having at least 80%, at least 85%, at least 90%, at least 95%, at least 96%, at least 97%, at least 98%, at least 99%, or 100% sequence identity to the amino acid sequence SEQ ID NO: 339; or
(i-4d) a heavy chain variable region (VH) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 336, and a light chain variable region (VL) comprising an amino acid sequence having at least 80%, at least 85%, at least 90%, at least 95%, at least 96%, at least 97%, at least 98%, at least 99%, or 100% sequence identity to the amino acid sequence SEQ ID NO: 339, wherein the VH comprises the HCDR1, HCDR2, and HCDR3 sequences set forth in: SEQ ID NO: 330, 331, and 332, respectively, and the VL comprises the LCDR1, LCDR2, and LCDR3 sequences set forth in: SEQ ID NO: 333, 338, and 335, respectively.

Thus, in some embodiments, the anti-PD-1 antibody or fragment thereof is a fragment of a humanized anti-PD-1 antibody comprising:
(i-4a) a heavy chain variable region (VH) comprising the HCDR1, HCDR2, and HCDR3 sequences of SEQ ID NO: 336, and a light chain variable region (VL) comprising the LCDR1, LCDR2, and LCDR3 sequences of SEQ ID NO: 341;
(i-4b) a heavy chain variable region (VH) comprising the HCDR1, HCDR2, and HCDR3 sequences set forth in: SEQ ID NO: 330, 331, and 332, respectively, and a light chain variable region (VL) comprising the LCDR1, LCDR2, and LCDR3 sequences set forth in: SEQ ID NO: 333, 340, and 335, respectively;
(i-4c) a heavy chain variable region (VH) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 336, and a light chain variable region (VL) comprising an amino acid sequence having at least 80%, at least 85%, at least 90%, at least 95%, at least 96%, at least 97%, at least 98%, at least 99%, or 100% sequence identity to the amino acid sequence SEQ ID NO: 341; or
(i-4d) a heavy chain variable region (VH) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 336, and a light chain variable region (VL) comprising an amino acid sequence having at least 80%, at least 85%, at least 90%, at least 95%, at least 96%, at least 97%, at least 98%, at least 99%, or 100% sequence identity to the amino acid sequence SEQ ID NO: 341, wherein the VH comprises the HCDR1, HCDR2, and HCDR3 sequences set forth in: SEQ ID NO: 330, 331, and 332, respectively, and the VL comprises the LCDR1, LCDR2, and LCDR3 sequences set forth in: SEQ ID NO: 333, 340, and 335, respectively.

Thus, in some embodiments, the anti-PD-1 antibody or fragment thereof is a fragment of a humanized anti-PD-1 antibody comprising:
(i-4a) a heavy chain variable region (VH) comprising the HCDR1, HCDR2, and HCDR3 sequences of SEQ ID NO: 336, and a light chain variable region (VL) comprising the LCDR1, LCDR2, and LCDR3 sequences of SEQ ID NO: 343;
(i-4b) a heavy chain variable region (VH) comprising the HCDR1, HCDR2, and HCDR3 sequences set forth in: SEQ ID NO: 330, 331, and 332, respectively, and a light chain variable region (VL) comprising the LCDR1, LCDR2, and LCDR3 sequences set forth in: SEQ ID NO: 333, 342, and 335, respectively;
(i-4c) a heavy chain variable region (VH) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 336, and a light chain variable region (VL) comprising an amino acid sequence having at least 80%, at least 85%, at least 90%, at least 95%, at least 96%, at least 97%, at least 98%, at least 99%, or 100% sequence identity to the amino acid sequence SEQ ID NO: 343; or
(i-4d) a heavy chain variable region (VH) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 336, and a light chain variable region (VL) comprising an amino acid sequence having at least 80%, at least 85%, at least 90%, at least 95%, at least 96%, at least 97%, at least 98%, at least 99%, or 100% sequence identity to the amino acid sequence SEQ ID NO: 343, wherein the VH comprises the HCDR1, HCDR2, and HCDR3 sequences set forth in: SEQ ID NO: 330, 331, and 332, respectively, and the VL comprises the LCDR1, LCDR2, and LCDR3 sequences set forth in: SEQ ID NO: 333, 342, and 335, respectively.

### A2. Anti-PD-L1 Antibodies or PD-L1 Binding Fragments Thereof

In some embodiments, the first binding region which binds to PD-1, PD-L1, or both and which antagonizes the PD-1/PD-L1 interaction comprises an anti-PD-L1 antibody or a PD-L1 binding fragment thereof.

In some embodiments, PD-L1 is human PD-L1, in particular human PD-L1 comprising the sequence set forth in SEQ ID NO: 172, 173, 174, or 175.

In some embodiments, the anti-PD-L1 antibody or a PD-L1 binding fragment thereof (anti-PD-L1 antibody or fragment thereof) specifically binds to PD-L1.

In some embodiments, the anti-PD-L1 antibody or fragment thereof is selected from the group consisting of atezolizumab, durvalumab, avelumab, and PD-L1 binding fragments thereof.

In some embodiments, the anti-PD-L1 antibody or fragment thereof is a monoclonal antibody or a fragment thereof.

In some embodiments, the anti-PD-L1 antibody or fragment thereof is a humanized antibody. In some embodiments, the anti-PD-L1 antibody or fragment thereof is a monoclonal humanized antibody. In some embodiments, the fragment of the anti-PD-L1 antibody may be any antigen-binding fragment of the anti-PD-L1 antibody, for example a Fab, a Fab′, a F(ab′)2, a Fv, a scFv, a Fab′-SH, an sdAb, or a VHH. In some embodiments, the anti-PD-L1 antibody or fragment thereof is selected from a full-length anti-PD-L1 antibody, an scFv of an anti-PD-L1 antibody or a VHH of an anti-PD-L1 antibody. In some embodiments, the anti-PD-L1 antibody or fragment thereof is a VHH of an anti-PD-L1 antibody.

In some embodiments, the anti-PD-L1 antibody or fragment thereof may comprise a variable region that specifically binds to PD-L1. In some embodiments, the anti-PD-L1 antibody or fragment thereof comprises a heavy chain variable region and/or a light chain variable domain. In some embodiments, the anti-PD-L1 antibody or fragment thereof comprises a heavy chain variable region and a light chain variable domain. In some embodiments, the anti-PD-L1 antibody or fragment thereof comprises a constant region, preferably derived from a human antibody, preferably the constant region is selected from the constant region of human IgG1, IgG2, IgG3 or IgG4. In some embodiments, the anti-PD-L1 antibody or fragment thereof comprises a heavy chain and/or a light chain. In some embodiments, the anti-PD-L1 antibody or fragment thereof comprises a heavy chain and a light chain. In some embodiments, the anti-PD-L1 antibody or fragment thereof comprises of a heavy chain and a light chain as a single chain (scFv). In some embodiments, the anti-PD-L1 antibody or fragment thereof is a VHH of an anti-PD-L1 antibody.

Exemplary anti-PD-L1 antibody CDRs, variable region sequences, and heavy and light chain sequences are provided in Table 1, e.g., for anti-PD-L1 antibodies A3 and A4, respectively.

In some embodiments, the anti-PD-L1 antibody or fragment thereof is defined by one or more sequences of the anti-PD-L1 antibody A3 shown in Table 1.

In some embodiments, the CDRs are defined according to the Kabat numbering system. Thus, in some embodiments, the anti-PD-L1 antibody or fragment thereof comprises: (i) a HCDR1 comprising or consisting of the amino acid sequence of SEQ ID NO: 120, or a variant thereof having up to three amino acid substitutions, additions or deletions; (ii) a HCDR2 comprising or consisting of the amino acid sequence of SEQ ID NO: 121, or a variant thereof having up to three amino acid substitutions, additions or deletions; and (iii) a HCDR3 comprising or consisting of the amino acid sequence of SEQ ID NO: 122, or a variant thereof having up to three amino acid substitutions, additions or deletions, wherein the CDRs are defined according to the Kabat numbering system. In some embodiments, the CDR variants have up to two amino acid substitutions, additions or deletions. In some embodiments, the CDR variants have up to one amino acid substitution, addition or deletion. In some embodiments, the CDR variants have up to three amino acid substitutions. In some embodiments, the CDR variants have up to two amino acid substitutions. In some embodiments, the CDR variants have up to one amino acid substitution.

In some embodiments, the CDRs are defined according to the IMGT numbering system.

Thus, in some embodiments, the anti-PD-L1 antibody or fragment thereof comprises: (i) a HCDR1 comprising or consisting of the amino acid sequence of SEQ ID NO: 123, or a variant thereof having up to three amino acid substitutions, additions or deletions; (ii) a HCDR2 comprising or consisting of the amino acid sequence of SEQ ID NO: 124, or a variant thereof having up to three amino acid substitutions, additions or deletions; and (iii) a HCDR3 comprising or consisting of the amino acid sequence of SEQ ID NO: 125, or a variant thereof having up to three amino acid substitutions, additions or deletions, wherein the CDRs are defined according to the IMGT numbering system. In some embodiments, the CDR variants have up to two amino acid substitutions, additions or deletions. In some embodiments, the CDR variants have up to one amino acid substitution, addition or deletion. In some embodiments, the CDR variants have up to three amino acid substitutions. In some embodiments, the CDR variants have up to two amino acid substitutions. In some embodiments, the CDR variants have up to one amino acid substitution.

In some embodiments, the anti-PD-L1 antibody or fragment thereof comprises or consists of an amino acid sequence having at least 80%, at least 85%, at least 90%, at least 95%, at least 96%, at least 97%, at least 98%, at least 99%, or 100% sequence identity to the amino acid sequence of SEQ ID NO: 126, and/or one or more (e.g., 1, 2, 3, 4, 5, 6, 7, 8, 9, or 10) amino acid additions, deletions and/or substitutions in a framework region thereof. In some embodiments, the anti-PD-L1 antibody or fragment thereof comprises or consists of an amino acid sequence having at least 80%, at least 85%, at least 90%, at least 95%, at least 96%, at least 97%, at least 98%, at least 99%, or 100% sequence identity to the amino acid sequence of SEQ ID NO: 126, and/or one or more (e.g., 1, 2, 3, 4, 5, 6, 7, 8, 9, or 10) amino acid additions, deletions and/or substitutions in a framework region thereof, wherein the amino acid sequence comprises (i) the HCDR1, HCDR2, and HCDR3 sequences set forth in: SEQ ID NO: 120, 121, and 122, respectively, or (ii) the HCDR1, HCDR2, and HCDR3 sequences set forth in: SEQ ID NO: 123, 124, and 125, respectively. In some embodiments, the anti-PD-L1 antibody or fragment thereof comprises or consists of an amino acid sequence having at least 80%, at least 85%, at least 90%, at least 95%, at least 96%, at least 97%, at least 98%, at least 99%, or 100% sequence identity to the amino acid sequence of SEQ ID NO: 126, wherein the amino acid sequence comprises (i) the HCDR1, HCDR2, and HCDR3 sequences set forth in: SEQ ID NO: 120, 121, and 122, respectively, or (ii) the HCDR1, HCDR2, and HCDR3 sequences set forth in: SEQ ID NO: 123, 124, and 125, respectively. In some embodiments, the anti-PD-L1 antibody or fragment thereof comprises or consists of the amino acid sequence of SEQ ID NO: 126.

Thus, in some embodiments, the anti-PD-L1 antibody or fragment thereof is a VHH comprising:
(i-3a) the HCDR1, HCDR2, and HCDR3 sequences set forth in: SEQ ID NO: 120, 121, and 122, respectively;
(i-3b) the HCDR1, HCDR2, and HCDR3 sequences set forth in: SEQ ID NO: 123, 124, and 125, respectively; or
(i-3c) the amino acid sequence of SEQ ID NO: 126 or an amino acid sequence having at least 80%, at least 85%, at least 90%, at least 95%, at least 96%, at least 97%, at least 98%, at least 99%, or 100% sequence identity to the amino acid sequence SEQ ID NO: 126, wherein the amino acid sequence having at least 80%, at least 85%, at least 90%, at least 95%, at least 96%, at least 97%, at least 98%, at least 99%, or 100% sequence identity to the amino acid sequence SEQ ID NO: 126 preferably comprises the HCDR1, HCDR2, and HCDR3 sequences set forth in: SEQ ID NO: 120, 121, and 122, respectively, or comprises the HCDR1, HCDR2, and HCDR3 sequences set forth in: SEQ ID NO: 123, 124, and 125, respectively.

In some alternative embodiments, the anti-PD-L1 antibody or fragment thereof is defined by one or more sequences of the anti-PD-L1 antibody A4 shown in Table 1.

In some embodiments, the anti-PD-L1 antibody or fragment thereof comprises (a) a heavy chain variable region (VH) comprising the CDR1, CDR2, and CDR3 sequences of SEQ ID NO: 133, and/or a light chain variable region (VL) comprising the CDR1, CDR2, and CDR3 sequences of SEQ ID NO: 134.

In some embodiments, (I) a VH of the anti-PD-L1 antibody or fragment thereof comprises: (i) a CDR1 comprising or consisting of the amino acid sequence of SEQ ID NO: 127, or a variant thereof having up to three amino acid substitutions, additions or deletions; (ii) a CDR2 comprising or consisting of the amino acid sequence of SEQ ID NO: 128, or a variant thereof having up to three amino acid substitutions, additions or deletions; and (iii) a CDR3 comprising or consisting of the amino acid sequence of SEQ ID NO: 129, or a variant thereof having up to three amino acid substitutions, additions or deletions, and/or (II) a VL of the anti-PD-1 antibody or fragment thereof comprises: (i) a CDR1 comprising or consisting of the amino acid sequence of SEQ ID NO: 130, or a variant thereof having up to three amino acid substitutions, additions or deletions; (ii) a CDR2 comprising or consisting of the amino acid sequence of SEQ ID NO: 131, or a variant thereof having up to three amino acid substitutions, additions or deletions; and (iii) a CDR3 comprising or consisting of the amino acid sequence of SEQ ID NO: 132, or a variant thereof having up to three amino acid substitutions, additions or deletions. In some embodiments, the CDR variants have up to two amino acid substitutions, additions or deletions. In some embodiments, the CDR variants have up to one amino acid substitution, addition or deletion. In some embodiments, the CDR variants have up to three amino acid substitutions. In some embodiments, the CDR variants have up to two amino acid substitutions. In some embodiments, the CDR variants have up to one amino acid substitution.

In some embodiments, the anti-PD-L1 antibody or fragment thereof comprises a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 133, and/or one or more (e.g., 1, 2, 3, 4, 5, 6, 7, 8, 9, or 10) amino acid additions, deletions and/or substitutions in a framework region thereof. In some embodiments, the anti-PD-L1 antibody or fragment thereof comprises a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 133, and/or one or more (e.g., 1, 2, 3, 4, 5, 6, 7, 8, 9, or 10) amino acid additions, deletions and/or substitutions in a framework region thereof, wherein the VH comprises the CDR1, CDR2, and CDR3 sequences set forth in: SEQ ID NO: 127, 128, and 129, respectively. In some embodiments, the anti-PD-L1 antibody or fragment thereof comprises a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 133. In some embodiments, the anti-PD-L1 antibody or fragment thereof comprises a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 133, wherein the VH comprises the CDR1, CDR2, and CDR3 sequences set forth in: SEQ ID NO: 127, 128, and 129, respectively. In some embodiments, the anti-PD-L1 antibody or fragment thereof comprises a VH comprising or consisting of the amino acid sequence of SEQ ID NO: 133.

In some embodiments, the anti-PD-L1 antibody or fragment thereof comprises a VL comprising or consisting of an amino acid sequence having at least 80%, at least 85%, at least 90%, at least 95%, at least 96%, at least 97%, at least 98%, at least 99%, or 100% sequence identity to the amino acid sequence SEQ ID NO: 134 and/or one or more (e.g., 1, 2, 3, 4, 5, 6, 7, 8, 9, or 10) amino acid additions, deletions and/or substitutions in a framework region thereof. In some embodiments, the anti-PD-L1 antibody or fragment thereof comprises a VL comprising or consisting of an amino acid sequence having at least 80%, at least 85%, at least 90%, at least 95%, at least 96%, at least 97%, at least 98%, at least 99%, or 100% sequence identity to the amino acid sequence SEQ ID NO: 134 and/or one or more (e.g., 1, 2, 3, 4, 5, 6, 7, 8, 9, or 10) amino acid additions, deletions and/or substitutions in a framework region thereof, wherein the VL comprises the CDR1, CDR2, and CDR3 sequences set forth in: SEQ ID NO: 130, 131, and 132, respectively. In some embodiments, the anti-PD-L1 antibody or fragment thereof comprises a VL comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 134. In some embodiments, the anti-PD-L1 antibody or fragment thereof comprises a VL comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 134, wherein the VL comprises the CDR1, CDR2, and CDR3 sequences set forth in: SEQ ID NO: 130, 131, and 132, respectively. In some embodiments, the anti-PD-L1 antibody or fragment thereof comprises a VL comprising or consisting of the amino acid sequence of SEQ ID NO: 134.

In some embodiments, the anti-PD-L1 antibody or fragment thereof comprises (I) a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 133, and/or one or more (e.g., 1, 2, 3, 4, 5, 6, 7, 8, 9, or 10) amino acid additions, deletions and/or substitutions in a framework region thereof, and (II) a VL comprising or consisting of an amino acid sequence having at least 80%, at least 85%, at least 90%, at least 95%, at least 96%, at least 97%, at least 98%, at least 99%, or 100% sequence identity to the amino acid sequence SEQ ID NO: 134 and/or one or more (e.g., 1, 2, 3, 4, 5, 6, 7, 8, 9, or 10) amino acid additions, deletions and/or substitutions in a framework region thereof. In some embodiments, the anti-PD-L1 antibody or fragment thereof comprises a (I) a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 133, and/or one or more (e.g., 1, 2, 3, 4, 5, 6, 7, 8, 9, or 10) amino acid additions, deletions and/or substitutions in a framework region thereof, and (II) a VL comprising or consisting of an amino acid sequence having at least 80%, at least 85%, at least 90%, at least 95%, at least 96%, at least 97%, at least 98%, at least 99%, or 100% sequence identity to the amino acid sequence SEQ ID NO: 134 and/or one or more (e.g., 1, 2, 3, 4, 5, 6, 7, 8, 9, or 10) amino acid additions, deletions and/or substitutions in a framework region thereof, wherein the VH comprises the CDR1, CDR2, and CDR3 sequences set forth in: SEQ ID NO: 127, 128, and 129, respectively, and the VL comprises the CDR1, CDR2, and CDR3 sequences set forth in: SEQ ID NO: 130, 131, and 132, respectively. In some embodiments, the anti-PD-L1 antibody or fragment thereof comprises (I) a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 133, and (II) a VL comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 134. In some embodiments, the anti-PD-L1 antibody or fragment thereof comprises (I) a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 133, and (II) a VL comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 134, wherein the VH comprises the CDR1, CDR2, and CDR3 sequences set forth in: SEQ ID NO: 127, 128, and 129, respectively, and the VL comprises the CDR1, CDR2, and CDR3 sequences set forth in: SEQ ID NO: 130, 131, and 132, respectively. In some embodiments, the anti-PD-L1 antibody or fragment thereof comprises (I) a VH comprising or consisting of the amino acid sequence of SEQ ID NO: 133, and (II) comprises a VL comprising or consisting of the amino acid sequence of SEQ ID NO: 134.

Thus, in some embodiments, the anti-PD-L1 antibody or fragment thereof is a fragment of a humanized anti-PD-L1 antibody comprising:
(i-4a) a heavy chain variable region (VH) comprising the HCDR1, HCDR2, and HCDR3 sequences of SEQ ID NO: 133, and a light chain variable region (VL) comprising the LCDR1, LCDR2, and LCDR3 sequences of SEQ ID NO: 134;
(i-4b) a heavy chain variable region (VH) comprising the HCDR1, HCDR2, and HCDR3 sequences set forth in: SEQ ID NO: 127, 128, and 129, respectively, and a light chain variable region (VL) comprising the LCDR1, LCDR2, and LCDR3 sequences set forth in: SEQ ID NO: 130, 131, and 132, respectively;
(i-4c) a heavy chain variable region (VH) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 133, and a light chain variable region (VL) comprising an amino acid sequence having at least 80%, at least 85%, at least 90%, at least 95%, at least 96%, at least 97%, at least 98%, at least 99%, or 100% sequence identity to the amino acid sequence SEQ ID NO: 134; or
(i-4d) a heavy chain variable region (VH) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 133, and a light chain variable region (VL) comprising an amino acid sequence having at least 80%, at least 85%, at least 90%, at least 95%, at least 96%, at least 97%, at least 98%, at least 99%, or 100% sequence identity to the amino acid sequence SEQ ID NO: 134, wherein the VH comprises the HCDR1, HCDR2, and HCDR3 sequences set forth in: SEQ ID NO: 127, 128, and 129, respectively, and the VL comprises the LCDR1, LCDR2, and LCDR3 sequences set forth in: SEQ ID NO: 130, 131, and 132, respectively.
   Second Binding Region which Binds to VEGF, VEGFR, or Both and which Antagonizes the VEGF/VEGFR Interaction

Another component of the anti-PD-1/PD-L1xVEGF/VEGFR binding agent is a second binding region which binds to VEGF, VEGFR, or both and which antagonizes the VEGF/VEGFR interaction. Thus, this second binding region comprises an antagonist of the VEGF/VEGFR interaction, such as an anti-VEGF antibody, a VEGF binding fragment thereof, a protein comprising a VEGFR domain, an anti-VEGFR antibody, or a VEGF binding fragment thereof.

### B1. Anti-VEGF Antibodies or VEGF Binding Fragments Thereof.

In some embodiments, the second binding region which binds to VEGF, VEGF receptor (VEGFR), or both and which antagonizes the VEGF/VEGFR interaction comprises an anti-VEGF antibody or a VEGF binding fragment thereof, preferably an anti-VEGF-A antibody or a VEGF-A binding fragment thereof.

In some embodiments, VEGF is human VEGF, in particular human VEGF comprising the sequence set forth in SEQ ID NO: 176 or human VEGF-A comprising the sequence set forth in SEQ ID NO: 177.

In some embodiments, the anti-VEGF antibody or a VEGF binding fragment thereof (anti-VEGF antibody or fragment thereof) specifically binds to VEGF, in particular to VEGF-A.

In some embodiments, the anti-VEGF antibody or fragment thereof is selected from the group consisting of bevacizumab, ranibizumab, and VEGF binding fragments thereof.

In some embodiments, the anti-VEGF antibody or fragment thereof is a monoclonal antibody or a fragment thereof.

In some embodiments, the anti-VEGF antibody or fragment thereof is a humanized antibody. In some embodiments, the anti-VEGF antibody or fragment thereof is a monoclonal humanized antibody. In some embodiments, the fragment of the anti-VEGF antibody may be any antigen-binding fragment of the anti-VEGF antibody, for example a Fab, a Fab′, a F(ab′)2, a Fv, a scFv, a Fab′-SH, an sdAb, or a VHH. In some embodiments, the anti-VEGF antibody or fragment thereof is selected from a full-length anti-VEGF antibody, an scFv of an anti-VEGF antibody, or a VHH of an anti-VEGF antibody. In some embodiments, the anti-VEGF antibody or fragment thereof is a full-length anti-VEGF antibody.

In some embodiments, the anti-VEGF antibody or fragment thereof may comprise a variable region that specifically binds to VEGF, in particular VEGF-A. In some embodiments, the anti-VEGF antibody or fragment thereof comprises a heavy chain variable region and/or a light chain variable domain. In some embodiments, the anti-VEGF antibody or fragment thereof comprises a heavy chain variable region and a light chain variable domain. In some embodiments, the anti-VEGF antibody or fragment thereof comprises a constant region, preferably derived from a human antibody, preferably the constant region is selected from the constant region of human IgG1, IgG2, IgG3 or IgG4. In some embodiments, the anti-VEGF antibody or fragment thereof comprises a heavy chain and/or a light chain. In some embodiments, the anti-VEGF antibody or fragment thereof comprises a heavy chain and a light chain. In some embodiments, the anti-VEGF antibody or fragment thereof comprises two heavy chains and two light chains.

Exemplary anti-VEGF antibody CDRs, variable region sequences, and heavy and light chain sequences are provided in Table 1; cf, e.g., SEQ ID NOs: 135 to 152.

In some embodiments, the anti-VEGF antibody or fragment thereof comprises a constant region, preferably derived from a human antibody, preferably the constant region is selected from the constant region of human IgG1, IgG2, IgG3 or IgG4.

In some embodiments, the anti-VEGF antibody or fragment thereof comprises (a) a heavy chain variable region (VH) comprising the CDR1, CDR2, and CDR3 sequences of SEQ ID NO: 146, and/or a light chain variable region (VL) comprising the CDR1, CDR2, and CDR3 sequences of SEQ ID NO: 147.

In some embodiments, the CDRs are defined according to the Kabat numbering system. Thus, in some embodiments, (I) a VH of the anti-VEGF antibody or fragment thereof comprises: (i) a CDR1 comprising or consisting of the amino acid sequence of SEQ ID NO: 135, or a variant thereof having up to three amino acid substitutions, additions or deletions; (ii) a CDR2 comprising or consisting of the amino acid sequence of SEQ ID NO: 136, or a variant thereof having up to three amino acid substitutions, additions or deletions; and (iii) a CDR3 comprising or consisting of the amino acid sequence of SEQ ID NO: 137, or a variant thereof having up to three amino acid substitutions, additions or deletions, and/or (II) a VL of the anti-VEGF antibody or fragment thereof comprises: (i) a CDR1 comprising or consisting of the amino acid sequence of SEQ ID NO: 138, or a variant thereof having up to three amino acid substitutions, additions or deletions; (ii) a CDR2 comprising or consisting of the amino acid sequence of SEQ ID NO: 139, or a variant thereof having up to three amino acid substitutions, additions or deletions; and (iii) a CDR3 comprising or consisting of the amino acid sequence of SEQ ID NO: 140, or a variant thereof having up to three amino acid substitutions, additions or deletions, wherein the CDRs are defined according to the Kabat numbering system. In some embodiments, the CDR variants have up to two amino acid substitutions, additions or deletions. In some embodiments, the CDR variants have up to one amino acid substitution, addition or deletion. In some embodiments, the CDR variants have up to three amino acid substitutions. In some embodiments, the CDR variants have up to two amino acid substitutions. In some embodiments, the CDR variants have up to one amino acid substitution.

In some embodiments, the CDRs are defined according to the IMGT numbering system. Thus, in some embodiments, (I) a VH of the anti-VEGF antibody or fragment thereof comprises: (i) a CDR1 comprising or consisting of the amino acid sequence of SEQ ID NO: 141, or a variant thereof having up to three amino acid substitutions, additions or deletions;
(ii) a CDR2 comprising or consisting of the amino acid sequence of SEQ ID NO: 142, or a variant thereof having up to three amino acid substitutions, additions or deletions; and (iii) a CDR3 comprising or consisting of the amino acid sequence of SEQ ID NO: 143, or a variant thereof having up to three amino acid substitutions, additions or deletions, and/or (II) a VL of the anti-VEGF antibody or fragment thereof comprises: (i) a CDR1 comprising or consisting of the amino acid sequence of SEQ ID NO: 144, or a variant thereof having up to three amino acid substitutions, additions or deletions; (ii) a CDR2 comprising or consisting of the amino acid sequence of SEQ ID NO: 145, or a variant thereof having up to one amino acid substitution, addition or deletion; and (iii) a CDR3 comprising or consisting of the amino acid sequence of SEQ ID NO: 140, or a variant thereof having up to three amino acid substitutions, additions or deletions, wherein the CDRs are defined according to the IMGT numbering system. In some embodiments, the CDR variants have up to two amino acid substitutions, additions or deletions. In some embodiments, the CDR variants have up to one amino acid substitution, addition or deletion. In some embodiments, the CDR variants have up to three amino acid substitutions. In some embodiments, the CDR variants have up to two amino acid substitutions. In some embodiments, the CDR variants have up to one amino acid substitution.

In some embodiments, the anti-VEGF antibody or fragment thereof comprises a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 146, and/or one or more (e.g., 1, 2, 3, 4, 5, 6, 7, 8, 9, or 10) amino acid additions, deletions and/or substitutions in a framework region thereof. In some embodiments, the anti-VEGF antibody or fragment thereof comprises a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 146, and/or one or more (e.g., 1, 2, 3, 4, 5, 6, 7, 8, 9, or 10) amino acid additions, deletions and/or substitutions in a framework region thereof, wherein the VH comprises the CDR1, CDR2, and CDR3 sequences set forth in: SEQ ID NO: 135, 136, and 137, respectively, or the VH comprises the CDR1, CDR2, and CDR3 sequences set forth in: SEQ ID NO: 141, 142, and 143, respectively. In some embodiments, the anti-VEGF antibody or fragment thereof comprises a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 146. In some embodiments, the anti-VEGF antibody or fragment thereof comprises a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 146, wherein the VH comprises the CDR1, CDR2, and CDR3 sequences set forth in: SEQ ID NO: 135, 136, and 137, respectively, or the VH comprises the CDR1, CDR2, and CDR3 sequences set forth in: SEQ ID NO: 141, 142, and 143, respectively. In some embodiments, the anti-VEGF antibody or fragment thereof comprises a VH comprising or consisting of the amino acid sequence of SEQ ID NO: 146.

In some embodiment, the anti-VEGF antibody or fragment thereof comprises a VL comprising or consisting of an amino acid sequence having at least 80%, at least 85%, at least 90%, at least 95%, at least 96%, at least 97%, at least 98%, at least 99%, or 100% sequence identity to the amino acid sequence SEQ ID NO: 147 and/or one or more (e.g., 1, 2, 3, 4, 5, 6, 7, 8, 9, or 10) amino acid additions, deletions and/or substitutions in a framework region thereof. In some embodiments, the anti-VEGF antibody or fragment thereof comprises a VL comprising or consisting of an amino acid sequence having at least 80%, at least 85%, at least 90%, at least 95%, at least 96%, at least 97%, at least 98%, at least 99%, or 100% sequence identity to the amino acid sequence SEQ ID NO: 147 and/or one or more (e.g., 1, 2, 3, 4, 5, 6, 7, 8, 9, or 10) amino acid additions, deletions and/or substitutions in a framework region thereof, wherein the VL comprises the CDR1, CDR2, and CDR3 sequences set forth in: SEQ ID NO: 138, 139, and 140, respectively, or the VL comprises the CDR1, CDR2, and CDR3 sequences set forth in: SEQ ID NO: 144, 145, and 140, respectively. In some embodiments, the anti-VEGF antibody or fragment thereof comprises a VL comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 147. In some embodiments, the anti-VEGF antibody or fragment thereof comprises a VL comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 147, wherein the VL comprises the CDR1, CDR2, and CDR3 sequences set forth in: SEQ ID NO: 138, 139, and 140, respectively, or the VL comprises the CDR1, CDR2, and CDR3 sequences set forth in: SEQ ID NO: 144, 145, and 140, respectively. In some embodiments, the anti-VEGF antibody or fragment thereof comprises a VL comprising or consisting of the amino acid sequence of SEQ ID NO: 147.

In some embodiments, the anti-VEGF antibody or fragment thereof comprises (I) a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 146, and/or one or more (e.g., 1, 2, 3, 4, 5, 6, 7, 8, 9, or 10) amino acid additions, deletions and/or substitutions in a framework region thereof, and (II) a VL comprising or consisting of an amino acid sequence having at least 80%, at least 85%, at least 90%, at least 95%, at least 96%, at least 97%, at least 98%, at least 99%, or 100% sequence identity to the amino acid sequence SEQ ID NO: 147 and/or one or more (e.g., 1, 2, 3, 4, 5, 6, 7, 8, 9, or 10) amino acid additions, deletions and/or substitutions in a framework region thereof. In some embodiments, the anti-VEGF antibody or fragment thereof comprisesa (I) a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 146, and/or one or more (e.g., 1, 2, 3, 4, 5, 6, 7, 8, 9, or 10) amino acid additions, deletions and/or substitutions in a framework region thereof, and (II) a VL comprising or consisting of an amino acid sequence having at least 80%, at least 85%, at least 90%, at least 95%, at least 96%, at least 97%, at least 98%, at least 99%, or 100% sequence identity to the amino acid sequence SEQ ID NO: 147 and/or one or more (e.g., 1, 2, 3, 4, 5, 6, 7, 8, 9, or 10) amino acid additions, deletions and/or substitutions in a framework region thereof, wherein either (i) the VH comprises the CDR1, CDR2, and CDR3 sequences set forth in: SEQ ID NO: 135, 136, and 137, respectively, and the VL comprises the CDR1, CDR2, and CDR3 sequences set forth in: SEQ ID NO: 138, 139, and 140, respectively, or (ii) the VH comprises the CDR1, CDR2, and CDR3 sequences set forth in: SEQ ID NO: 141, 142, and 143, respectively, and the VL comprises the CDR1, CDR2, and CDR3 sequences set forth in: SEQ ID NO: 144, 145, and 140, respectively. In some embodiments, the anti-VEGF antibody or fragment thereof comprises (I) a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 146, and (II) a VL comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 147. In some embodiments, the anti-VEGF antibody or fragment thereof comprises (I) a VH comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 146, and (II) a VL comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 147, wherein either (i) the VH comprises the CDR1, CDR2, and CDR3 sequences set forth in: SEQ ID NO: 135, 136, and 137, respectively, and the VL comprises the CDR1, CDR2, and CDR3 sequences set forth in: SEQ ID NO: 138, 139, and 140, respectively, or (ii) the VH comprises the CDR1, CDR2, and CDR3 sequences set forth in: SEQ ID NO: 141, 142, and 143, respectively, and the VL comprises the CDR1, CDR2, and CDR3 sequences set forth in: SEQ ID NO: 144, 145, and 140, respectively. In some embodiments, the anti-VEGF antibody or fragment thereof comprises (I) a VH comprising or consisting of the amino acid sequence of SEQ ID NO: 146, and (II) comprises a VL comprising or consisting of the amino acid sequence of SEQ ID NO: 147.

In some embodiments, the anti-VEGF antibody or fragment thereof comprises a CH1 domain, preferably comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 148.

In some embodiments, the anti-VEGF antibody or fragment thereof comprises an Fc region comprising one or more mutations which reduce or abolish immune effect functions. Suitable mutations are known in the art and may include one or more amino acid substitutions (cf, e.g., Wilkinson, I., et al., 2021. FcPLoS One, 16(12), p.e0260954; and Liu, R., et al., 2020. Antibodies, 9(4), p.64). In some embodiments, the anti-VEGF antibody or fragment thereof comprises an IgG1 Fc region comprising a L234A and/or a L235A mutation. In some embodiments, the anti-VEGF antibody or fragment thereof comprises an IgG1 Fc region comprising L234A and L235A mutations. Amino acids are numbered according to the EU numbering (EU-index) as set forth in Kabat (Kabat, E. A.; National Institutes of Health (U.S.) Office of the Director. Sequences of Proteins of Immunological Interest, 5th ed.; DIANE Publishing: Collingdale, PA, USA, 1991). For example, these may correspond to L240A and L241A in SEQ ID NO: 151. In some embodiments, the anti-VEGF antibody or fragment thereof comprises a Fc domain comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 149.

In some embodiments, the anti-VEGF antibody or fragment thereof comprises a CL domain, preferably comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 150.

In some embodiments, the anti-VEGF antibody or fragment thereof comprises a heavy chain (HC) comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 151, and/or one or more (e.g., 1, 2, 3, 4, 5, 6, 7, 8, 9, or 10) amino acid additions, deletions and/or substitutions in a framework region thereof. In some embodiments, the anti-VEGF antibody or fragment thereof comprises an HC comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 151, and/or one or more (e.g., 1, 2, 3, 4, 5, 6, 7, 8, 9, or 10) amino acid additions, deletions and/or substitutions in a framework region thereof, wherein the HC comprises the CDR1, CDR2, and CDR3 sequences set forth in: SEQ ID NO: 135, 136, and 137, respectively, or the HC comprises the CDR1, CDR2, and CDR3 sequences set forth in: SEQ ID NO: 141, 142, and 143, respectively. In some embodiments, the anti-VEGF antibody or fragment thereof comprises an HC comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 151. In some embodiments, the anti-VEGF antibody or fragment thereof comprises an HC comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 151, wherein the HC comprises the CDR1, CDR2, and CDR3 sequences set forth in: SEQ ID NO: 135, 136, and 137, respectively, or the HC comprises the CDR1, CDR2, and CDR3 sequences set forth in: SEQ ID NO: 141, 142, and 143, respectively. In some embodiments, the anti-VEGF antibody or fragment thereof comprises an HC comprising or consisting of the amino acid sequence of SEQ ID NO: 151.

In some embodiments, the anti-VEGF antibody or fragment thereof comprises a light chain (LC) comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 152, and/or one or more (e.g., 1, 2, 3, 4, 5, 6, 7, 8, 9, or 10) amino acid additions, deletions and/or substitutions in a framework region thereof. In some embodiments, the anti-VEGF antibody or fragment thereof comprises an LC comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 152, and/or one or more (e.g., 1, 2, 3, 4, 5, 6, 7, 8, 9, or 10) amino acid additions, deletions and/or substitutions in a framework region thereof, wherein the LC comprises the CDR1, CDR2, and CDR3 sequences set forth in: SEQ ID NO: 138, 139, and 140, respectively, or the LC comprises the CDR1, CDR2, and CDR3 sequences set forth in: SEQ ID NO: 144, 145, and 140, respectively. In some embodiments, the anti-VEGF antibody or fragment thereof comprises an LC comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 152. In some embodiments, the anti-VEGF antibody or fragment thereof comprises an LC comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 152, wherein the LC comprises the CDR1, CDR2, and CDR3 sequences set forth in: SEQ ID NO: 138, 139, and 140, respectively, or the LC comprises the CDR1, CDR2, and CDR3 sequences set forth in: SEQ ID NO: 144, 145, and 140, respectively. In some embodiments, the anti-VEGF antibody or fragment thereof comprises an LC comprising or consisting of the amino acid sequence of SEQ ID NO: 152.

In some embodiments, the anti-VEGF antibody or fragment thereof comprises (I) a heavy chain (HC) comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 151, and/or one or more (e.g., 1, 2, 3, 4, 5, 6, 7, 8, 9, or 10) amino acid additions, deletions and/or substitutions in a framework region thereof, and (II) a light chain (LC) comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 152, and/or one or more (e.g., 1, 2, 3, 4, 5, 6, 7, 8, 9, or 10) amino acid additions, deletions and/or substitutions in a framework region thereof. In some embodiments, the anti-VEGF antibody or fragment thereof comprises (I) an HC comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 151, and/or one or more (e.g., 1, 2, 3, 4, 5, 6, 7, 8, 9, or 10) amino acid additions, deletions and/or substitutions in a framework region thereof, and (II) an LC comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 152, and/or one or more (e.g., 1, 2, 3, 4, 5, 6, 7, 8, 9, or 10) amino acid additions, deletions and/or substitutions in a framework region thereof, wherein either (i) the HC comprises the CDR1, CDR2, and CDR3 sequences set forth in: SEQ ID NO: 135, 136, and 137, respectively, and the LC comprises the CDR1, CDR2, and CDR3 sequences set forth in: SEQ ID NO:138 139, and 140, respectively, or (ii) the HC comprises the CDR1, CDR2, and CDR3 sequences set forth in: SEQ ID NO: 141, 142, and 143, respectively, and the LC comprises the CDR1, CDR2, and CDR3 sequences set forth in: SEQ ID NO: 144, 145, and 140, respectively. In some embodiments, the anti-VEGF antibody or fragment thereof comprises (I) an HC comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 151, and (II) an LC comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 152. In some embodiments, the anti-VEGF antibody or fragment thereof comprises (I) an HC comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 151, and (II) an LC comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 152, wherein either (i) the HC comprises the CDR1, CDR2, and CDR3 sequences set forth in: SEQ ID NO: 135, 136, and 137, respectively, and the LC comprises the CDR1, CDR2, and CDR3 sequences set forth in: SEQ ID NO: 138, 139, and 140, respectively, or (ii) the HC comprises the CDR1, CDR2, and CDR3 sequences set forth in: SEQ ID NO: 141, 142, and 143, respectively, and the LC comprises the CDR1, CDR2, and CDR3 sequences set forth in: SEQ ID NO: 144, 145, and 140, respectively. In some embodiments, the anti-VEGF antibody or fragment thereof comprises (I) an HC comprising or consisting of the amino acid sequence of SEQ ID NO: 151, and (II) an LC comprising or consisting of the amino acid sequence of SEQ ID NO: 152.

In some embodiments, the anti-VEGF antibody comprises or consists of two HCs each comprising the amino acid sequence of SEQ ID NO: 151; and two LCs each comprising the amino acid sequence of SEQ ID NO: 152.

Thus, in some embodiments, the anti-VEGF antibody or fragment thereof is a full-length humanized anti-VEGF antibody comprising:
(ii-1a) a heavy chain variable region (VH) comprising the HCDR1, HCDR2, and HCDR3 sequences of SEQ ID NO: 146, and a light chain variable region (VL) comprising the LCDR1, LCDR2, and LCDR3 sequences of SEQ ID NO: 147;
(ii-1b) a heavy chain variable region (VH) comprising the HCDR1, HCDR2, and HCDR3 sequences set forth in: SEQ ID NO: 135, 136, and 137, respectively, and a light chain variable region (VL) comprising the CDR1, CDR2, and CDR3 sequences set forth in: SEQ ID NO: 138, 139, and 140, respectively;
(ii-1c) a heavy chain variable region (VH) comprising the HCDR1, HCDR2, and HCDR3 sequences set forth in: SEQ ID NO: 141, 142, and 143, respectively, and a light chain variable region (VL) comprising the LCDR1, LCDR2, and LCDR3 sequences set forth in: SEQ ID NO: 144, 145, and 140, respectively;
(ii-1d) a heavy chain variable region (VH) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 146, and a light chain variable region (VL) comprising an amino acid sequence having at least 80%, at least 85%, at least 90%, at least 95%, at least 96%, at least 97%, at least 98%, at least 99%, or 100% sequence identity to the amino acid sequence SEQ ID NO: 147;
(ii-1e) a heavy chain variable region (VH) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 146, and a light chain variable region (VL) comprising an amino acid sequence having at least 80%, at least 85%, at least 90%, at least 95%, at least 96%, at least 97%, at least 98%, at least 99%, or 100% sequence identity to the amino acid sequence SEQ ID NO: 147, wherein the VH comprises the HCDR1, HCDR2, and HCDR3 sequences set forth in: SEQ ID NO: 135, 136, and 137, respectively, and the VL comprises the LCDR1, LCDR2, and LCDR3 sequences set forth in: SEQ ID NO: 138, 139, and 140, respectively;
(ii-1f) a heavy chain variable region (VH) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 146, and a light chain variable region (VL) comprising an amino acid sequence having at least 80%, at least 85%, at least 90%, at least 95%, at least 96%, at least 97%, at least 98%, at least 99%, or 100% sequence identity to the amino acid sequence SEQ ID NO: 147, wherein the VH comprises the HCDR1, HCDR2, and HCDR3 sequences set forth in: SEQ ID NO: 141, 142, and 143, respectively, and the VL comprises the LCDR1, LCDR2, and LCDR3 sequences set forth in: SEQ ID NO: 144, 145, and 140, respectively;
(ii-1g) a heavy chain (HC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 151 and a light chain (LC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 152;
(ii-1h) a heavy chain (HC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 151 and a light chain (LC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 152, wherein the HC comprises the HCDR1, HCDR2, and HCDR3 sequences set forth in: SEQ ID NO: 135, 136, and 137, respectively, and the LC comprises the LCDR1, LCDR2, and LCDR3 sequences set forth in: SEQ ID NO: 138, 139, and 140, respectively; or
(ii-1i) a heavy chain (HC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 151 and a light chain (LC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 152, wherein the HC comprises the HCDR1, HCDR2, and HCDR3 sequences set forth in: SEQ ID NO: 141, 142, and 143, respectively, and the LC comprises the LCDR1, LCDR2, and LCDR3 sequences set forth in: SEQ ID NO: 144, 145, and 140, respectively.

### B2. VEGFR Domains

In some embodiments, the second binding region which binds to VEGF, VEGF receptor (VEGFR), or both and which antagonizes the VEGF/VEGFR interaction comprises a VEGFR domain. In some embodiments, a protein which comprises a VEGFR domain is Aflibercept. Exemplary sequences of VEGFR domains are provided in Table 1.

In some embodiments, the second binding region which binds to VEGF, VEGF receptor (VEGFR), or both and which antagonizes the VEGF/VEGFR interaction comprises at least one VEGFR-1 domain, or at least one VEGFR-2 domain, or at least one VEGFR-3 domain, or at least one VEGFR-1 domain and at least one VEGFR-2 domain, such as two VEGFR-1 domains, or two VEGFR-2 domains, or two VEGFR-1 domains and two VEGFR-2 domains, or two VEGFR-3 domains.

In some embodiments, the VEGFR domain is VEGFR-1 comprising or consisting of the amino acid sequence set forth in SEQ ID NO: 153, or is an amino acid sequence having at least 80%, at least 85%, at least 90%, at least 95%, at least 96%, at least 97%, at least 98%, at least 99%, or 100% sequence identity to the amino acid sequence of SEQ ID NO: 153.

In some embodiments, the VEGFR domain is VEGFR-2 comprising or consisting of the amino acid sequence set forth in: SEQ ID NO: 154, or is an amino acid sequence having at least 80%, at least 85%, at least 90%, at least 95%, at least 96%, at least 97%, at least 98%, at least 99%, or 100% sequence identity to the amino acid sequence of SEQ ID NO: 154.

In some embodiments, the VEGFR domain is VEGFR-3 comprising or consisting of the amino acid sequence set forth in SEQ ID NO: 155, or is an amino acid sequence having at least 80%, at least 85%, at least 90%, at least 95%, at least 96%, at least 97%, at least 98%, at least 99%, or 100% sequence identity to the amino acid sequence of SEQ ID NO: 155.

In some embodiments, the second binding region comprises at least one VEGFR-1 domain comprising or consisting of at least one amino acid sequence having at least 80%, at least 85%, at least 90%, at least 95%, at least 96%, at least 97%, at least 98%, at least 99%, or 100% sequence identity to the amino acid sequence of SEQ ID NO: 153.

In some embodiments, the second binding region comprises at least one VEGFR-2 domain comprising or consisting of at least one amino acid sequence having at least 80%, at least 85%, at least 90%, at least 95%, at least 96%, at least 97%, at least 98%, at least 99%, or 100% sequence identity to the amino acid sequence of SEQ ID NO: 154.

In some embodiments, the second binding region comprises at least one VEGFR-3 domain comprising or consisting of at least one amino acid sequence having at least 80%, at least 85%, at least 90%, at least 95%, at least 96%, at least 97%, at least 98%, at least 99%, or 100% sequence identity to the amino acid sequence of SEQ ID NO: 155.

In some preferred embodiments, the second binding region comprises (i) at least one VEGFR-1 domain or at least one amino acid sequence having at least 80%, at least 85%, at least 90%, at least 95%, at least 96%, at least 97%, at least 98%, at least 99%, or 100% sequence identity to the amino acid sequence of SEQ ID NO: 153, or at least one VEGFR-3 domain or at least one amino acid sequence having at least 80%, at least 85%, at least 90%, at least 95%, at least 96%, at least 97%, at least 98%, at least 99%, or 100% sequence identity to the amino acid sequence of SEQ ID NO: 155, and (ii) at least one VEGFR-2 domain or at least one amino acid sequence having at least 80%, at least 85%, at least 90%, at least 95%, at least 96%, at least 97%, at least 98%, at least 99%, or 100% sequence identity to the amino acid sequence of SEQ ID NO: 154. In particularly preferred embodiments, the second binding region comprises (i) two VEGFR-1 domains or two amino acid sequences each having at least 80%, at least 85%, at least 90%, at least 95%, at least 96%, at least 97%, at least 98%, at least 99%, or 100% sequence identity to the amino acid sequence of SEQ ID NO: 153, (ii) two VEGFR-2 domains or two amino acid sequences having at least 80%, at least 85%, at least 90%, at least 95%, at least 96%, at least 97%, at least 98%, at least 99%, or 100% sequence identity to the amino acid sequence of SEQ ID NO: 154, (iii) two VEGFR-3 domains or two amino acid sequences each having at least 80%, at least 85%, at least 90%, at least 95%, at least 96%, at least 97%, at least 98%, at least 99%, or 100% sequence identity to the amino acid sequence of SEQ ID NO: 155, (iv) a combination of (i) and (ii), or (v) a combination of (ii) and (iii).

In some preferred embodiments, the second binding region comprises or consists of the amino acid sequence of SEQ ID NO: 156 or an amino acid sequence having at least 80%, at least 85%, at least 90%, at least 95%, at least 96%, at least 97%, at least 98%, at least 99%, or 100% sequence identity to the amino acid sequence of SEQ ID NO: 156.

In some embodiments, the second binding region comprises or consists of (i) at least one VEGFR-1 or VEGFR-3 domain or at least one amino acid sequence having at least 80%, at least 85%, at least 90%, at least 95%, at least 96%, at least 97%, at least 98%, at least 99%, or 100% sequence identity to the amino acid sequence of SEQ ID NO: 153 or 155; (ii) at least one VEGFR-2 domain or at least one amino acid sequence having at least 80%, at least 85%, at least 90%, at least 95%, at least 96%, at least 97%, at least 98%, at least 99%, or 100% sequence identity to the amino acid sequence of SEQ ID NO: 154; and (iii) an Fc part. For example, the second binding region may comprise or consist of the amino acid sequence of SEQ ID NO: 157 or an amino acid sequence having at least 80%, at least 85%, at least 90%, at least 95%, at least 96%, at least 97%, at least 98%, at least 99%, or 100% sequence identity to the amino acid sequence of SEQ ID NO: 157.

In some embodiments, the second binding region comprises:
(ii-2a) the amino acid sequence of SEQ ID NO: 153 or an amino acid sequence having at least 80%, at least 85%, at least 90%, at least 95%, at least 96%, at least 97%, at least 98%, at least 99%, or 100% sequence identity to the amino acid sequence of SEQ ID NO: 153;
(ii-2b) the amino acid sequence of SEQ ID NO: 154 or an amino acid sequence having at least 80%, at least 85%, at least 90%, at least 95%, at least 96%, at least 97%, at least 98%, at least 99%, or 100% sequence identity to the amino acid sequence of SE Q ID NO: 154;
(ii-2c) the amino acid sequence of SEQ ID NO: 155 or an amino acid sequence having at least 80%, at least 85%, at least 90%, at least 95%, at least 96%, at least 97%, at least 98%, at least 99%, or 100% sequence identity to the amino acid sequence of SEQ ID NO: 155;
(ii-2d) the amino acid sequence of SEQ ID NO: 156 or an amino acid sequence having at least 80%, at least 85%, at least 90%, at least 95%, at least 96%, at least 97%, at least 98%, at least 99%, or 100% sequence identity to the amino acid sequence of SEQ ID NO: 156; or
(ii-2e) the amino acid sequence of SEQ ID NO: 157 or an amino acid sequence having at least 80%, at least 85%, at least 90%, at least 95%, at least 96%, at least 97%, at least 98%, at least 99%, or 100% sequence identity to the amino acid sequence of SEQ ID NO: 157.

In some embodiments, the second binding region comprises two amino acid sequences each comprising or consisting of the amino acid sequence of SEQ ID NO: 157. The resulting second binding region may have the setup of an antibody when it comes to the Fc parts, i.e., the two Fc parts form an Fc domain via at least one disulfide bond, and the two N-terminal regions each comprise or consist of two VEGFR domains. Accordingly, the resulting second binding region may have an Fc domain and two VEGFR-containing arms, wherein each arm comprises two VEGFR domains such that the resulting second binding region overall comprises four VEGFR domains.

### B3. Anti-VEGFR Antibodies or VEGFR Binding Fragments Thereof

In some embodiments, the second binding region which binds to VEGF, VEGF receptor (VEGFR), or both and which antagonizes the VEGF/VEGFR interaction comprises an anti-VEGFR antibody or a VEGFR binding fragment thereof, preferably an anti-VEGFR2 antibody or a VEGFR2 binding fragment thereof.

In some embodiments, VEGFR is human VEGFR2, in particular human VEGFR2 comprising the sequence set forth in SEQ ID NO: 344 or monkey VEGFR2 comprising the sequence set forth in SEQ ID NO: 345.

In some embodiments, the anti-VEGFR antibody or a VEGFR binding fragment thereof (anti-VEGFR antibody or fragment thereof) specifically binds to a VEGFR, in particular to VEGFR2.

In some embodiments, the anti-VEGFR antibody or fragment thereof is selected from the group consisting of Ramucirumab (Cyramza®, Eli Lilly), AK-109 (AkesoBio), Gentuximab (Changchun Genescience Pharmaceuticals Co, Ltd.), BC-001 (Buchang Pharmaceuticals Co Ltd.), JY-025 (Beijing Dongfang Baitai Biotechnology Co Ltd), KD-035 (Sanofi), Olinvacimab (PharmAbcine Inc), and VEGFR binding fragments thereof.

In some embodiments, the anti-VEGFR antibody or fragment thereof is a monoclonal antibody or a fragment thereof.

In some embodiments, the anti-VEGFR antibody or fragment thereof is a humanized antibody. In some embodiments, the anti-VEGFR antibody or fragment thereof is a monoclonal humanized antibody. In some embodiments, the fragment of the anti-VEGFR antibody may be any antigen-binding fragment of the anti-VEGF antibody, for example a Fab, a Fab′, a F(ab′)2, a Fv, a scFv, a Fab′-SH, an sdAb, or a VHH. In some embodiments, the anti-VEGFR antibody or fragment thereof is selected from a full-length anti-VEGFR antibody, an scFv of an anti-VEGFR antibody, or a VHH of an anti-VEGFR antibody. In some embodiments, the anti-VEGFR antibody or fragment thereof is a full-length anti-VEGFR antibody.

In some embodiments, the anti-VEGFR antibody or fragment thereof may comprise a variable region that specifically binds to VEGFR, in particular VEGFR2. In some embodiments, the anti-VEGFR antibody or fragment thereof comprises a heavy chain variable region and/or a light chain variable domain. In some embodiments, the anti-VEGFR antibody or fragment thereof comprises a heavy chain variable region and a light chain variable domain. In some embodiments, the anti-VEGFR antibody or fragment thereof comprises a constant region, preferably derived from a human antibody, preferably the constant region is selected from the constant region of human IgG1, IgG2, IgG3 or IgG4. In some embodiments, the anti-VEGFR antibody or fragment thereof comprises a heavy chain and/or a light chain. In some embodiments, the anti-VEGFR antibody or fragment thereof comprises a heavy chain and a light chain. In some embodiments, the anti-VEGFR antibody or fragment thereof comprises two heavy chains and two light chains.

### Specific Embodiments of the Anti-PD-1 PD-L1xVEGF VEGFR Binding Agent

In some embodiments, the anti-PD-1/PD-L1xVEGF/VEGFR binding agent comprises a multispecific antibody, preferably a bispecific antibody.

The anti-PD-1/PD-L1xVEGF/VEGFR binding agent can comprise any of the first and second binding regions as outlined above, in any combination. Thus, the anti-PD-1/PD-L1xVEGF/VEGFR binding agent may comprise (i) a PD-1 binding antibody or PD-1 binding fragment thereof, such as pembrolizumab, nivolumab, cemiplimab, dostarlimab, toripalimab, and retifanlimab, or a fragment of any of the afore-mentioned antibodies, or any of the PD-1 binding sequences as disclosed above; and (ii) a VEGF binding antibody or VEGFR-binding fragment thereof, such as bevacizumab, ramucirumab, and ranibizumab, or a fragment of any of the afore-mentioned antibodies, or any of the VEGF-binding sequences as disclosed above; or a VEGFR domain or a protein comprising the same, such as aflibercept, or any of the VEGFR sequences as disclosed above. Alternatively, the anti-PD-1/PD-L1xVEGF/VEGFR binding agent may comprise (i) a PD-L1 binding antibody or PD-L1 binding fragment thereof, such as atezolizumab, durvalumab, and avelumab, or a fragment of any of the afore-mentioned antibodies, or any of the PD-L1 binding sequences as disclosed above; and (ii) a VEGF binding antibody or VEGFR-binding fragment thereof, such as bevacizumab, ramucirumab, and ranibizumab, or a fragment of any of the afore-mentioned antibodies, or any of the VEGF-binding sequences as disclosed above; or a VEGFR domain or a protein comprising the same, such as Aflibercept, or any of the VEGFR sequences as disclosed above.

In a particularly preferred embodiment, the anti-PD-1/PD-L1xVEGF/VEGFR binding agent comprises (i) the PD-L1 binding sequences, such as the VHH sequences and the anti-PD-L1 HCDRs thereof as disclosed above; and (ii) a VEGF binding antibody or VEGFR-binding fragment thereof as disclosed above, such as bevacizumab.

Exemplary bispecific anti-PD-1/PD-L1xVEGF/VEGFR binding agents include the following: the anti-PD-1/PD-L1xVEGF/VEGFR binding agents C1, C2a, C2b, C2c, C2d, C3a, and C3b provided in Table 1, Ivonescimab/AK112 (AkesoBio/Summit Therapeutics), AI-081 (OncoC4), SSGJ-707 (3SBio), LM-299 (LaNova Medicines/Merck), SG1408 (Hangzhou Sumgen), B1962 (AP Biosciences/Tasly Pharma), JS-207 (Junshi Biosciences), SCTB14 (Sinocelltech), CVL006 (Convalife), and MHB039A (Minghui Pharma). In one embodiment, the bispecific antibody is selected from the group consisting of the anti-PD-1/PD-L1xVEGF/VEGFR binding agents C1, C2a, C2b, C2c, C2d, C3a, and C3b provided in Table 1, ivonescimab/AK112 (AkesoBio/Summit Therapeutics), SSGJ-707 (3SBio), LM-299 (LaNova Medicines/Merck), SG1408 (Hangzhou Sumgen), B1962 (AP Biosciences/Tasly Pharma), JS-207 (Junshi Biosciences), SCTB14 (Sinocelltech), CVL006 (Convalife), and MHB039A (Minghui Pharma). Preferred anti-PD-1/PD-L1xVEGF/VEGFR binding agents are the anti-PD-1/PD-L1xVEGF/VEGFR binding agents C1, C2a, C2b, C2c, C2d, C3a, and C3b, in particular, anti-PD-1/PD-L1xVEGF/VEGFR binding agent C1.

In some embodiments, the antibody comprises any combination of one or more full-length antibodies, such as a human antibody, a humanized antibody, a chimeric antibody, a monoclonal antibody, or a polyclonal antibody, and/or one or more antibody fragments, such as a Fab, a Fab′, a F(ab′)2, an Fv, an scFv, a Fab′-SH, an sdAb, or a VHH. In some embodiments, the antibody comprises a full-length antibody and an antibody fragment, such as a Fab, a Fab′, a F(ab′)2, an Fv, an scFv, a Fab′-SH, an sdAb, or a VHH.

In some embodiments, the first binding region comprises an anti-PD-1 antibody or fragment thereof, or comprises an antibody fragment selected from an Fab, an Fab′, an F(ab′)2, an Fv, an scFv, an Fab′-SH, an sdAb, and a VHH, preferably an scFv or a VHH. In some embodiments, the anti-PD-1 antibody or fragment thereof is an anti-PD-1 single-chain variable fragment. In some embodiments, the anti-PD-1 antibody or fragment thereof is an anti-PD-1 VHH.

In some embodiments, the first binding region comprises an anti-PD-L1 antibody or fragment thereof, or comprises an antibody fragment selected from an Fab, an Fab′, an F(ab′)2, an Fv, an scFv, an Fab′-SH, an sdAb, and a VHH, preferably a sdAb, most preferably a VHH. In some embodiments, the anti-PD-L1 antibody or fragment thereof is an anti-PD-L1 single domain antibody. In some embodiments, the anti-PD-L1 antibody or fragment thereof is an anti-PD-L1 VHH.

In some embodiments, the second binding region comprises an anti-VEGF antibody or fragment thereof, or comprises a human antibody, a humanized antibody, or a chimeric antibody, preferably a humanized antibody. In some embodiments, the anti-VEGF antibody or fragment thereof is a monoclonal antibody, for example a monoclonal human antibody, a monoclonal humanized antibody, or a monoclonal chimeric antibody. In some embodiments, the anti-VEGF antibody or fragment thereof is a monoclonal humanized antibody.

In some embodiments, the multispecific (preferably bispecific) anti-PD-1/PD-L1xVEGF/VEGFR binding agent comprises an anti-VEGF antibody (e.g., anti-VEGF-A antibody) and/or an anti-PD-L1 single domain antibody. In some embodiments, the anti-PD-1/PD-L1xVEGF/VEGFR binding agent comprises an anti-VEGF antibody (e.g., anti-VEGF-A antibody) and/or an anti-PD-1 single-chain variable fragment.

In some embodiments, the multispecific (preferably bispecific) anti-PD-1/PD-L1xVEGF/VEGFR binding agent has monovalent binding to VEGF and/or to PD-L1. In some embodiments, the bispecific antibody has monovalent binding to VEGF and/or to PD-1. In some embodiment the multispecific (preferably bispecific) anti-PD-1/PD-L1xVEGF/VEGFR binding agent has bivalent binding to VEGF and/or to PD-L1. In some embodiment the multispecific (preferably bispecific) anti-PD-1/PD-L1xVEGF/VEGFR binding agent has bivalent binding to VEGF and/or to PD-1. In some preferred embodiments, the multispecific (preferably bispecific) anti-PD-1/PD-L1xVEGF/VEGFR binding agent has bivalent binding to VEGF and to PD-L1. In some preferred embodiments, the multispecific (preferably bispecific) anti-PD-1/PD-L1xVEGF/VEGFR binding agent has bivalent binding to VEGF and to PD-1.

In some embodiments, the anti-VEGF antibody (e.g., anti-VEGF-A antibody) or fragment thereof is a monovalent or bivalent antibody or fragment thereof. In some embodiments, the anti-PD-L1 antibody or fragment thereof is a monovalent or bivalent antibody or fragment thereof. In some embodiments, the anti-PD-1 antibody or fragment thereof is a monovalent or bivalent antibody or fragment thereof. In some preferred embodiments, the anti-VEGF antibody (e.g., anti-VEGF-A antibody) or fragment thereof is a bivalent anti-VEGF antibody or fragment thereof and/or the anti-PD-L1 antibody or fragment thereof is a bivalent antibody or fragment thereof. In some preferred embodiments, the anti-VEGF antibody (e.g., anti-VEGF-A antibody) or fragment thereof is a bivalent anti-VEGF antibody or fragment thereof and/or the anti-PD-1 antibody or fragment thereof is a bivalent antibody or fragment thereof.

In some preferred embodiments, the anti-VEGF antibody (e.g., anti-VEGF-A antibody) is a bivalent anti-VEGF antibody and the anti-PD-L1 is a monovalent single domain antibody. In some preferred embodiments, the anti-VEGF antibody (e.g., anti-VEGF-A antibody) is a bivalent anti-VEGF antibody and the anti-PD-1 is a bivalent single-chain variable fragment.

### C1. Anti-PD-L1xVEGF Antibodies

In some preferred embodiments, the anti-PD-1/PD-L1xVEGF/VEGFR binding agent is a bispecific anti-PD-L1xVEGF antibody, i.e., a bispecific antibody, wherein the first binding region of the bispecific antibody binds to PD-L1 and the second binding region binds to VEGF, preferably to VEGF-A. An exemplary and preferred bispecific anti-PD-L1xVEGF antibody is the anti-PD-1/PD-L1xVEGF/VEGFR binding agent C1 provided in Table 1.

In some embodiments, the anti-PD-L1xVEGF antibody comprises an anti-VEGF antibody as described herein and at least one anti-PD-L1 single domain antibody as described herein, preferably an anti-VEGF antibody as described herein and two anti-PD-L1 single domain antibodies as described herein, more preferably an anti-VEGF antibody as described herein and two anti-PD-L1 VHHs as described herein.

In some embodiments of the anti-PD-L1xVEGF antibody, the at least one anti-PD-L1 single domain antibody may be fused to the anti-VEGF antibody. In some embodiments, each anti-PD-L1 single domain antibody is terminally fused to the anti-VEGF antibody. In some preferred embodiments, each anti-PD-L1 single domain antibody is fused to: (i) the C-terminus of a heavy chain; (ii) the N-terminus of a heavy chain; (iii) the C-terminus of a light chain; or (iv) the N-terminus of a light chain. In particularly preferred embodiments, each anti-PD-L1 single domain antibody is fused to the C-terminus of an anti-VEGF antibody, preferably the C-terminus of a heavy chain. The anti-PD-L1xVEGF antibody more preferably comprises an anti-VEGF antibody and two anti-PD-L1 VHHs, wherein each heavy chain of the anti-VEGF antibody is fused to an anti-PD-L1 VHH, more preferably at the C-terminus of the heavy chain of the anti-VEGF antibody. The anti-PD-L1 single domain antibody (in particular, VHH) can be fused to the anti-VEGF antibody by a linker, e.g., a peptide linker, for example a glycine and serine-rich linker (GS-linker) or any suitable linker known in the art, such as linker a comprising or consisting of the amino acid sequence GGGGSGGGGSG (SEQ ID NO: 166).

In preferred embodiments, the anti-PD-L1xVEGF antibody comprises a linker, which more preferably comprises or consists of the amino acid sequence of SEQ ID NO: 166 or an amino acid sequence having at least 80%, 85%, 90%, 95%, 96%, 97%, 98%, 99%, or 100% identity to the amino acid sequence of SEQ ID NO: 166.

In particularly preferred embodiments, the anti-PD-L1xVEGF antibody comprises (i) an anti-VEGF antibody comprising two heavy chains as described herein; and (ii) two anti-PD-L1 VHHs, wherein each heavy chain of the anti-VEGF antibody is fused to an anti-PD-L1 VHH through a linker comprising the amino acid sequence of SEQ ID NO: 166 or an amino acid sequence having at least 80%, 85%, 90%, 95%, 96%, 97%, 98%, 99%, or 100% identity to the amino acid sequence of SEQ ID NO: 166.

In particularly preferred embodiments, the anti-PD-L1xVEGF antibody comprises two anti-PD-L1 VHHs as described herein, wherein each VHH is fused to the C-terminus of an anti-VEGF-A antibody as described herein, preferably the C-terminus of each heavy chain of the anti-VEGF-A antibody. The anti-PD-L1xVEGF antibody can therefore comprise an anti-VEGF-A antibody as described herein and two anti-PD-L1 VHHs as described herein, wherein each heavy chain of the anti-VEGF-A antibody is fused to an anti-PD-L1 VHH through a linker, such as a linker comprising the amino acid sequence shown in SEQ ID NO: 166 or an amino acid sequence having at least 80%, 85%, 90%, 95%, 96%, 97%, 98%, 99%, or 100% identity to the amino acid sequence of SEQ ID NO: 166.

In particularly preferred embodiments, the anti-PD-L1xVEGF antibody comprises (i) an anti-VEGF antibody or fragment thereof comprising the HCDRs and LCDRs as described above (i.e., SEQ ID NOs: 135-140 (Kabat) or SEQ ID NOs: 141-145 and 140 (IMGT)) or variants thereof having up to three amino acid substitutions, additions or deletions; and (ii) an anti-PD-L1 single domain comprising the HCDRs as described above (i.e., SEQ ID NOs: 120-122 (Kabat) or SEQ ID NOs: 123-125 (IMGT)), or variants thereof having up to three amino acid substitutions, additions or deletions. In some embodiments, the anti-PD-L1xVEGF antibody comprises (1) at least one anti-PD-L1 single domain antibody comprising or consisting of the amino acid sequence of SEQ ID NO: 126 or an amino acid sequence having at least 80%, at least 85%, at least 90%, at least 95%, at least 96%, at least 97%, at least 98%, at least 99%, or 100% sequence identity to the amino acid sequence SEQ ID NO: 126, wherein the amino acid sequence having at least 80%, at least 85%, at least 90%, at least 95%, at least 96%, at least 97%, at least 98%, at least 99%, or 100% sequence identity to the amino acid sequence SEQ ID NO: 126 preferably comprises the HCDR1, HCDR2, and HCDR3 sequences set forth in: SEQ ID NO: 120, 121, and 122, respectively, or the HCDR1, HCDR2, and HCDR3 sequences set forth in: 123, 124, and 125, respectively; and/or (2) an anti-VEGF antibody comprising (i) a heavy chain variable region (VH) comprising the HCDR1, HCDR2, and HCDR3 sequences set forth in: SEQ ID NO: 135, 136, and 137, respectively, and a light chain variable region (VL) comprising the CDR1, CDR2, and CDR3 sequences set forth in: SEQ ID NO: 138, 139, and 140, respectively; or (ii) a VH comprising the HCDR1, HCDR2, and HCDR3 sequences set forth in: SEQ ID NO: 141, 142, and 143, respectively, and a VL comprising the LCDR1, LCDR2, and LCDR3 sequences set forth in: SEQ ID NO: 144, 145, and 140, respectively.

In some embodiments, the CDR variants have up to two amino acid substitutions, additions or deletions. In some embodiments, the CDR variants have up to one amino acid substitution, addition or deletion. In some embodiments, the CDR variants have up to three amino acid substitutions. In some embodiments, the CDR variants have up to two amino acid substitutions. In some embodiments, the CDR variants have up to one amino acid substitution.

In some embodiments, the anti-VEGF antibody comprises:
(ii-1d) a heavy chain variable region (VH) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 146, and a light chain variable region (VL) comprising an amino acid sequence having at least 80%, at least 85%, at least 90%, at least 95%, at least 96%, at least 97%, at least 98%, at least 99%, or 100% sequence identity to the amino acid sequence SEQ ID NO: 147;
(ii-1e) a heavy chain variable region (VH) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 146, and a light chain variable region (VL) comprising an amino acid sequence having at least 80%, at least 85%, at least 90%, at least 95%, at least 96%, at least 97%, at least 98%, at least 99%, or 100% sequence identity to the amino acid sequence SEQ ID NO: 147, wherein the VH comprises the HCDR1, HCDR2, and HCDR3 sequences set forth in: SEQ ID NO: 135, 136, and 137, respectively, and the VL comprises the LCDR1, LCDR2, and LCDR3 sequences set forth in: SEQ ID NO: 138, 139, and 140, respectively;
(ii-1f) a heavy chain variable region (VH) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 146, and a light chain variable region (VL) comprising an amino acid sequence having at least 80%, at least 85%, at least 90%, at least 95%, at least 96%, at least 97%, at least 98%, at least 99%, or 100% sequence identity to the amino acid sequence SEQ ID NO: 147, wherein the VH comprises the HCDR1, HCDR2, and HCDR3 sequences set forth in: SEQ ID NO: 141, 142, and 143, respectively, and the VL comprises the LCDR1, LCDR2, and LCDR3 sequences set forth in: SEQ ID NO: 144, 145, and 140, respectively;
(ii-1g) a heavy chain (HC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 151, and a light chain (LC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 152;
(ii-1h) a heavy chain (HC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 151, and a light chain (LC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 152, wherein the HC comprises the HCDR1, HCDR2, and HCDR3 sequences set forth in: SEQ ID NO: 135, 136, and 137, respectively, and the LC comprises the LCDR1, LCDR2, and LCDR3 sequences set forth in: SEQ ID NO: 138, 139, and 140, respectively; or
(ii-1i) a heavy chain (HC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 151, and a light chain (LC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 152, wherein the HC comprises the HCDR1, HCDR2, and HCDR3 sequences set forth in: SEQ ID NO: 141, 142, and 143, respectively, and the LC comprises the LCDR1, LCDR2, and LCDR3 sequences set forth in: SEQ ID NO: 144, 145, and 140, respectively.

In particularly preferred embodiments, the anti-PD-L1xVEGF antibody comprises (i) a heavy chain sequence comprising or consisting of the amino acid sequence of SEQ ID NO: 158 or an amino acid sequence having at least 80%, 85%, 90%, 95%, 96%, 97%, 98%, 99%, or 100% identity to the amino acid sequence of SEQ ID NO: 158, and/or one or more (e.g., 1, 2, 3, 4, 5, 6, 7, 8, 9, or 10) amino acid additions, deletions and/or substitutions in a framework region thereof; and (ii) a light chain sequence comprising or consisting of the amino acid sequence of SEQ ID NO: 152 or an amino acid sequence having at least 80%, 85%, 90%, 95%, 96%, 97%, 98%, 99%, or 100% identity to the amino acid sequence of SEQ ID NO: 152, and/or one or more (e.g., 1, 2, 3, 4, 5, 6, 7, 8, 9, or 10) amino acid additions, deletions and/or substitutions in a framework region thereof. In some embodiments, the anti-PD-L1xVEGF antibody comprises (A) a heavy chain (HC) comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 158, and (B) a light chain (LC) comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 152, wherein the HC comprises (i) the HCDR1, HCDR2, and HCDR3 sequences set forth in: SEQ ID NO: 135, 136, and 137, respectively, and (ii) the HCDR1, HCDR2, and HCDR3 sequences set forth in: SEQ ID NO: 120, 121, and 122, respectively, and the LC comprises the LCDR1, LCDR2, and LCDR3 sequences set forth in: SEQ ID NO: 138, 139, and 140, respectively. In some alternative embodiments, the anti-PD-L1xVEGF antibody comprises (A′) a heavy chain (HC) comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 158, and (B′) a light chain (LC) comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 152, wherein the HC comprises (i) the HCDR1, HCDR2, and HCDR3 sequences set forth in: SEQ ID NO: 141, 142, and 143, respectively, and (ii) the HCDR1, HCDR2, and HCDR3 sequences set forth in: SEQ ID NO: 123, 124, and 125, respectively, and the LC comprises the LCDR1, LCDR2, and LCDR3 sequences set forth in: SEQ ID NO: 144, 145, and 140, respectively.

In some particularly preferred embodiments, the anti-PD-L1xVEGF antibody comprises (i) a heavy chain sequence comprising or consisting of the amino acid sequence of SEQ ID NO: 158 or an amino acid sequence having at least 80%, 85%, 90%, 95%, 96%, 97%, 98%, 99%, or 100% identity to the amino acid sequence of SEQ ID NO: 158; and (ii) a light chain sequence comprising or consisting of the amino acid sequence of SEQ ID NO: 152 or an amino acid sequence having at least 80%, 85%, 90%, 95%, 96%, 97%, 98%, 99%, or 100% identity to the amino acid sequence of SEQ ID NO: 152.

In some particularly preferred embodiments, the anti-PD-L1xVEGF antibody comprises (i) a heavy chain sequence comprising or consisting of the amino acid sequence of SEQ ID NO: 158; and (ii) a light chain sequence comprising or consisting of the amino acid sequence of SEQ ID NO: 152. In some particularly preferred embodiments, the anti-PD-L1xVEGF antibody comprises (i) two heavy chains each comprising or consisting of the amino acid sequence of SEQ ID NO: 158; and (ii) two light chains each comprising or consisting of the amino acid sequence of SEQ ID NO: 152. Such an anti-PD-L1xVEGF antibody comprising two heavy chain-VHH fusions having the amino acid sequence of SEQ ID NO: 158 and two light chains having the amino acid sequence of SEQ ID NO: 152 is disclosed as compound “Ava-2GS-NSD” in WO 2022/042719.

### C2. Anti-PD-1xVEGF Antibodies

In some embodiments, the anti-PD-1/PD-L1xVEGF/VEGFR binding agent is a bispecific anti-PD-1xVEGF antibody, i.e., a bispecific antibody, wherein the first binding region of the bispecific antibody binds to PD-1 and the second binding region binds to VEGF, preferably to VEGF-A. Exemplary bispecific anti-PD-1xVEGF antibodies are the anti-PD-1/PD-L1xVEGF/VEGFR binding agents C2a, C2b, C2c, and C2d provided in Table 1.

In some embodiments, the anti-PD-1xVEGF antibody comprises an anti-VEGF antibody as described herein and at least one anti-PD-1 scFv as described herein, preferably an anti-VEGF antibody as described herein and at least two anti-PD-1 scFvs as described herein, more preferably an anti-VEGF antibody as described herein and two anti-PD-1 scFvs as described herein.

In some embodiments of the anti-PD-1xVEGF antibody, the at least one anti-PD-1 scFv may be fused to the anti-VEGF antibody. In some embodiments, each anti-PD-1 scFv is terminally fused to the anti-VEGF antibody. In some preferred embodiments, each anti-PD-1 scFv is fused to: (i) the C-terminus of a heavy chain; (ii) the N-terminus of a heavy chain; (iii) the C-terminus of a light chain; or (iv) the N-terminus of a light chain. In particularly preferred embodiments, each anti-PD-1 scFv is fused to the C-terminus of an anti-VEGF antibody, preferably the C-terminus of a heavy chain. The anti-PD-1xVEGF antibody more preferably comprises an anti-VEGF antibody and two anti-PD-1 scFvs, wherein each heavy chain of the anti-VEGF antibody is fused to an anti-PD-1 scFv, more preferably at the C-terminus of the heavy chain of the anti-VEGF antibody. The anti-PD-1 scFv can be fused to the anti-VEGF antibody by a linker, e.g., a peptide linker, for example a glycine and serine-rich linker (GS-linker) or any suitable linker known in the art, such as linker a comprising or consisting of the amino acid sequence GGGGSGGGGSGGGGSGGGGS (SEQ ID NO: 167).

In preferred embodiments, the anti-PD-1xVEGF antibody comprises a linker, which more preferably comprises or consists of the amino acid sequence of SEQ ID NO: 167 or an amino acid sequence having at least 80%, 85%, 90%, 95%, 96%, 97%, 98%, 99%, or 100% identity to the amino acid sequence of SEQ ID NO: 167.

In particularly preferred embodiments, the anti-PD-1xVEGF antibody comprises (i) an anti-VEGF antibody comprising two heavy chains as described herein; and (ii) two anti-PD-1 scFvs, wherein each heavy chain of the anti-VEGF antibody is fused to an anti-PD-1 scFv through a linker, wherein the linker preferably comprises or consists of the amino acid sequence of SEQ ID NO: 167 or an amino acid sequence having at least 80%, 85%, 90%, 95%, 96%, 97%, 98%, 99%, or 100% identity to the amino acid sequence of SEQ ID NO: 167.

In particularly preferred embodiments, the anti-PD-1xVEGF antibody comprises two anti-PD-1 scFvs as described herein, wherein each anti-PD-1 scFv is fused to the C-terminus of an anti-VEGF-A antibody as described herein, preferably the C-terminus of each heavy chain of the anti-VEGF-A antibody. The anti-PD-1xVEGF antibody can therefore comprise an anti-VEGF-A antibody as described herein and two anti-PD-1 scFvs as described herein, wherein each heavy chain of the anti-VEGF-A antibody is fused to an anti-PD-1 scFv through a linker, such as a linker comprising or consisting of the amino acid sequence shown in SEQ ID NO: 167 or an amino acid sequence having at least 80%, 85%, 90%, 95%, 96%, 97%, 98%, 99%, or 100% identity to the amino acid sequence of SEQ ID NO: 167.

In particularly preferred embodiments, the anti-PD-1xVEGF antibody comprises (i) an anti-VEGF antibody or fragment thereof comprising the HCDRs and LCDRs as described above (i.e., SEQ ID NOs: 135-140 (Kabat) or SEQ ID NOs: 141-145 and 140 (IMGT)) or variants thereof having up to three amino acid substitutions, additions or deletions; and (ii) an anti-PD-1 scFv comprising the HCDRs and LCDRs as described above (i.e., SEQ ID NOs: 184-188 and 183 (Kabat) or SEQ ID NOs: 178-183 (IMGT)), or variants thereof having up to three amino acid substitutions, additions or deletions. In some embodiments, the anti-PD-1xVEGF antibody comprises (1) at least one anti-PD-1 scFv comprising or consisting of the amino acid sequence of SEQ ID NO: 189 or an amino acid sequence having at least 80%, at least 85%, at least 90%, at least 95%, at least 96%, at least 97%, at least 98%, at least 99%, or 100% sequence identity to the amino acid sequence SEQ ID NO: 189, wherein the amino acid sequence having at least 80%, at least 85%, at least 90%, at least 95%, at least 96%, at least 97%, at least 98%, at least 99%, or 100% sequence identity to the amino acid sequence SEQ ID NO: 189 preferably comprises the HCDR1, HCDR2, HCDR3, LCDR1, LCDR2, and LCDR3 sequences set forth in: SEQ ID NO: 184, 185, 186, 187, 188, and 183, respectively, or the HCDR1, HCDR2, HCDR3, LCDR1, LCDR2, and LCDR3 sequences set forth in: 178, 179, 180, 181, 182, and 183, respectively; and/or (2) an anti-VEGF antibody comprising (i) a heavy chain variable region (VH) comprising the HCDR1, HCDR2, and HCDR3 sequences set forth in: SEQ ID NO: 135, 136, and 137, respectively, and a light chain variable region (VL) comprising the CDR1, CDR2, and CDR3 sequences set forth in: SEQ ID NO: 138, 139, and 140, respectively; or (ii) a VH comprising the HCDR1, HCDR2, and HCDR3 sequences set forth in: SEQ ID NO: 141, 142, and 143, respectively, and a VL comprising the LCDR1, LCDR2, and LCDR3 sequences set forth in: SEQ ID NO: 144, 145, and 140, respectively.

In some embodiments, the CDR variants have up to two amino acid substitutions, additions or deletions. In some embodiments, the CDR variants have up to one amino acid substitution, addition or deletion. In some embodiments, the CDR variants have up to three amino acid substitutions. In some embodiments, the CDR variants have up to two amino acid substitutions. In some embodiments, the CDR variants have up to one amino acid substitution.

In some embodiments, the anti-VEGF antibody comprises (a) the VH and VL sequences as specified above under (ii-1d), (ii-1e), or (ii-1f), or (b) the HC and LC sequences as specified above under (ii-1g), (ii-1h), or (ii-1i).

In particularly preferred embodiments, the anti-PD-1xVEGF antibody comprises (i) a heavy chain sequence comprising or consisting of the amino acid sequence of SEQ ID NO: 159 or an amino acid sequence having at least 80%, 85%, 90%, 95%, 96%, 97%, 98%, 99%, or 100% identity to the amino acid sequence of SEQ ID NO: 159, and/or one or more (e.g., 1, 2, 3, 4, 5, 6, 7, 8, 9, or 10) amino acid additions, deletions and/or substitutions in a framework region thereof; and (ii) a light chain sequence comprising or consisting of the amino acid sequence of SEQ ID NO: 152 or an amino acid sequence having at least 80%, 85%, 90%, 95%, 96%, 97%, 98%, 99%, or 100% identity to the amino acid sequence of SEQ ID NO: 152, and/or one or more (e.g., 1, 2, 3, 4, 5, 6, 7, 8, 9, or 10) amino acid additions, deletions and/or substitutions in a framework region thereof. In some embodiments, the anti-PD-1xVEGF antibody comprises (A) a heavy chain (HC) comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 159, and (B) a light chain (LC) comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 152, wherein the HC comprises (i) the HCDR1, HCDR2, and HCDR3 sequences set forth in: SEQ ID NO: 135, 136, and 137, respectively, and (ii) the HCDR1, HCDR2, HCDR3, LCDR1, LCDR2, and LCDR3 sequences set forth in: SEQ ID NO: 184, 185, 186, 187, 188, and 183, respectively, and the LC comprises the LCDR1, LCDR2, and LCDR3 sequences set forth in: SEQ ID NO: 138, 139, and 140, respectively. In some alternative embodiments, the anti-PD-L1xVEGF antibody comprises (A′) a heavy chain (HC) comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 159, and (B′) a light chain (LC) comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 152, wherein the HC comprises (i) the HCDR1, HCDR2, and HCDR3 sequences set forth in: SEQ ID NO: 141, 142, and 143, respectively, and (ii) the HCDR1, HCDR2, HCDR3, LCDR1, LCDR2, and LCDR3 sequences set forth in: SEQ ID NO: 178, 179, 180, 181, 182, and 183, respectively, and the LC comprises the LCDR1, LCDR2, and LCDR3 sequences set forth in: SEQ ID NO: 144, 145, and 140, respectively.

In some particularly preferred embodiments, the anti-PD-1xVEGF antibody comprises or consists of (i) a heavy chain sequence comprising or consisting of the amino acid sequence of SEQ ID NO: 159 or an amino acid sequence having at least 80%, 85%, 90%, 95%, 96%, 97%, 98%, 99%, or 100% identity to the amino acid sequence of SEQ ID NO: 159; and (ii) a light chain sequence comprising or consisting of the amino acid sequence of SEQ ID NO: 152 or an amino acid sequence having at least 80%, 85%, 90%, 95%, 96%, 97%, 98%, 99%, or 100% identity to the amino acid sequence of SEQ ID NO: 152.

In some particularly preferred embodiments, the anti-PD-1xVEGF antibody comprises (i) a heavy chain sequence comprising or consisting of the amino acid sequence of SEQ ID NO: 159; and (ii) a light chain sequence comprising or consisting of the amino acid sequence of SEQ ID NO: 152. In some particularly preferred embodiments, the anti-PD-1xVEGF antibody comprises or consists of (i) two heavy chains each comprising or consisting of the amino acid sequence of SEQ ID NO: 159; and (ii) two light chains each comprising or consisting of the amino acid sequence of SEQ ID NO: 152.

In some embodiments, the anti-PD-1xVEGF antibody comprises an anti-VEGF antibody as described herein and at least one anti-PD-1 VHH as described herein, preferably an anti-VEGF antibody as described herein and at least two anti-PD-1 VHHs as described herein, more preferably an anti-VEGF antibody as described herein and two or four anti-PD-1 VHHs as described herein.

In some embodiments of the anti-PD-1xVEGF antibody, the at least one anti-PD-1 VHH may be fused to the anti-VEGF antibody. In some embodiments, each anti-PD-1 VHH is terminally fused to the anti-VEGF antibody. In some preferred embodiments, each anti-PD-1 VHH is fused to: (i) the C-terminus of a heavy chain; (ii) the N-terminus of a heavy chain; (iii) the C-terminus of a light chain; or (iv) the N-terminus of a light chain. In particularly preferred embodiments, each anti-PD-1 VHH is fused to the C-terminus of an anti-VEGF antibody, preferably the C-terminus of a heavy chain. The anti-PD-1xVEGF antibody more preferably comprises an anti-VEGF antibody and two anti-PD-1 VHH, wherein each heavy chain of the anti-VEGF antibody is fused to an anti-PD-1 VHH, more preferably at the C-terminus of the heavy chain of the anti-VEGF antibody. The anti-PD-1 VHH can be fused to the anti-VEGF antibody by a linker, e.g., a peptide linker, for example a glycine and serine-rich linker (GS-linker) or any suitable linker known in the art, such as linker a comprising or consisting of the amino acid sequence GGGGSGGGGSGGGGSGGGGS (SEQ ID NO: 167).

In preferred embodiments, the anti-PD-1xVEGF antibody comprises a linker, which more preferably comprises or consists of the amino acid sequence of SEQ ID NO: 167 or an amino acid sequence having at least 80%, 85%, 90%, 95%, 96%, 97%, 98%, 99%, or 100% identity to the amino acid sequence of SEQ ID NO: 167.

In particularly preferred embodiments, the anti-PD-1xVEGF antibody comprises (i) an anti-VEGF antibody comprising or consisting of two heavy chains as described herein; and (ii) at least two anti-PD-1 VHHs, wherein each heavy chain of the anti-VEGF antibody is fused to an anti-PD-1 VHH through a linker comprising or consisting of the amino acid sequence of SEQ ID NO: 167 or an amino acid sequence having at least 80%, 85%, 90%, 95%, 96%, 97%, 98%, 99%, or 100% identity to the amino acid sequence of SEQ ID NO: 167.

In particularly preferred embodiments, the anti-PD-1xVEGF antibody comprises two or four anti-PD-1 VHHs as described herein, wherein each anti-PD-1 VHH is fused to the C-terminus of an anti-VEGF-A antibody as described herein, preferably the C-terminus of each heavy chain of the anti-VEGF-A antibody. The anti-PD-1xVEGF antibody can therefore comprise an anti-VEGF-A antibody as described herein and two anti-PD-1 VHHs as described herein, wherein each heavy chain of the anti-VEGF-A antibody is fused to an anti-PD-1 VHH through a linker, such as a linker comprising or consisting of the amino acid sequence shown in SEQ ID NO: 167 or an amino acid sequence having at least 80%, 85%, 90%, 95%, 96%, 97%, 98%, 99%, or 100% identity to the amino acid sequence of SEQ ID NO: 167.

In particularly preferred embodiments, the anti-PD-1xVEGF antibody comprises (i) an anti-VEGF antibody or fragment thereof comprising the HCDRs and LCDRs as described above (i.e., SEQ ID NOs: 135-140 (Kabat) or SEQ ID NOs: 141-145 and 140 (IMGT)) or variants thereof having up to three amino acid substitutions, additions or deletions; and (ii) an anti-PD-1 VHH comprising the HCDRs as described above (i.e., SEQ ID NOs: 110, 111, and 112 or 113 (Kabat) or SEQ ID NOs: 114, 115, and 116, or 117 (IMGT)), or variants thereof having up to three amino acid substitutions, additions or deletions. In some embodiments, the anti-PD-1xVEGF antibody comprises (1) at least one anti-PD-1 VHH comprising or consisting of the amino acid sequence of SEQ ID NO: 118 or 119 or an amino acid sequence having at least 80%, at least 85%, at least 90%, at least 95%, at least 96%, at least 97%, at least 98%, at least 99%, or 100% sequence identity to the amino acid sequence SEQ ID NO: 118 or 119, wherein the amino acid sequence having at least 80%, at least 85%, at least 90%, at least 95%, at least 96%, at least 97%, at least 98%, at least 99%, or 100% sequence identity to the amino acid sequence SEQ ID NO: 118 preferably comprises the HCDR1, HCDR2, and HCDR3 sequences set forth in: SEQ ID NO: 110, 111, and 112, respectively, or the HCDR1, HCDR2, and HCDR3 sequences set forth in: 114, 115, and 116, respectively, and the amino acid sequence having at least 80%, at least 85%, at least 90%, at least 95%, at least 96%, at least 97%, at least 98%, at least 99%, or 100% sequence identity to the amino acid sequence SEQ ID NO: 119 preferably comprises the HCDR1, HCDR2, and HCDR3 sequences set forth in: SEQ ID NO: 110, 111, and 113, respectively, or the HCDR1, HCDR2, and HCDR3 sequences set forth in: 114, 115, and 117, respectively; and/or (2) an anti-VEGF antibody comprising (i) a heavy chain variable region (VH) comprising the HCDR1, HCDR2, and HCDR3 sequences set forth in: SEQ ID NO: 135, 136, and 137, respectively, and a light chain variable region (VL) comprising the CDR1, CDR2, and CDR3 sequences set forth in: SEQ ID NO: 138, 139, and 140, respectively; or (ii) a VH comprising the HCDR1, HCDR2, and HCDR3 sequences set forth in: SEQ ID NO: 141, 142, and 143, respectively, and a VL comprising the LCDR1, LCDR2, and LCDR3 sequences set forth in: SEQ ID NO: 144, 145, and 140, respectively.

In some embodiments, the CDR variants have up to two amino acid substitutions, additions or deletions. In some embodiments, the CDR variants have up to one amino acid substitution, addition or deletion. In some embodiments, the CDR variants have up to three amino acid substitutions. In some embodiments, the CDR variants have up to two amino acid substitutions. In some embodiments, the CDR variants have up to one amino acid substitution.

In some embodiments, the anti-VEGF antibody comprises (a) the VH and VL sequences as specified above under (ii-1d), (ii-1e), or (ii-1f), or (b) the HC and LC sequences as specified above under (ii-1g), (ii-1h), or (ii-1i).

In one preferred embodiment, the anti-PD-1xVEGF antibody comprises (i) a heavy chain sequence comprising or consisting of the amino acid sequence of SEQ ID NO: 160 or an amino acid sequence having at least 80%, 85%, 90%, 95%, 96%, 97%, 98%, 99%, or 100% identity to the amino acid sequence of SEQ ID NO: 160, and/or one or more (e.g., 1, 2, 3, 4, 5, 6, 7, 8, 9, or 10) amino acid additions, deletions and/or substitutions in a framework region thereof, and (ii) a light chain sequence comprising or consisting of the amino acid sequence of SEQ ID NO: 152 or an amino acid sequence having at least 80%, 85%, 90%, 95%, 96%, 97%, 98%, 99%, or 100% identity to the amino acid sequence of SEQ ID NO: 152, and/or one or more (e.g., 1, 2, 3, 4, 5, 6, 7, 8, 9, or 10) amino acid additions, deletions and/or substitutions in a framework region thereof. In some embodiments, the anti-PD-1xVEGF antibody comprises (A) a heavy chain (HC) comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 160, and (B) a light chain (LC) comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 152, wherein the HC comprises (i) the HCDR1, HCDR2, and HCDR3 sequences set forth in: SEQ ID NO: 135, 136, and 137, respectively, and (ii) the HCDR1, HCDR2, and HCDR3 sequences set forth in: SEQ ID NO: 110, 111, and 112, respectively, and the LC comprises the LCDR1, LCDR2, and LCDR3 sequences set forth in: SEQ ID NO: 138, 139, and 140, respectively. In some alternative embodiments, the anti-PD-1xVEGF antibody comprises (A′) a heavy chain (HC) comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 160, and (B′) a light chain (LC) comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 152, wherein the HC comprises (i) the HCDR1, HCDR2, and HCDR3 sequences set forth in: SEQ ID NO: 141, 142, and 143, respectively, and (ii) the HCDR1, HCDR2, and HCDR3 sequences set forth in: SEQ ID NO: 114, 115, and 116, respectively, and the LC comprises the LCDR1, LCDR2, and LCDR3 sequences set forth in: SEQ ID NO: 144, 145, and 140, respectively.

In some preferred embodiments, the anti-PD-1xVEGF antibody comprises or consists of (i) a heavy chain sequence comprising or consisting of the amino acid sequence of SEQ ID NO: 160 or an amino acid sequence having at least 80%, 85%, 90%, 95%, 96%, 97%, 98%, 99%, or 100% identity to the amino acid sequence of SEQ ID NO: 160; and (ii) a light chain sequence comprising or consisting of the amino acid sequence of SEQ ID NO: 152 or an amino acid sequence having at least 80%, 85%, 90%, 95%, 96%, 97%, 98%, 99%, or 100% identity to the amino acid sequence of SEQ ID NO: 152.

In a second preferred embodiment, the anti-PD-1xVEGF antibody comprises (i) a heavy chain sequence comprising or consisting of the amino acid sequence of SEQ ID NO: 161 or an amino acid sequence having at least 80%, 85%, 90%, 95%, 96%, 97%, 98%, 99%, or 100% identity to the amino acid sequence of SEQ ID NO: 161, and/or one or more (e.g., 1, 2, 3, 4, 5, 6, 7, 8, 9, or 10) amino acid additions, deletions and/or substitutions in a framework region thereof, and (ii) a light chain sequence comprising or consisting of the amino acid sequence of SEQ ID NO: 152 or an amino acid sequence having at least 80%, 85%, 90%, 95%, 96%, 97%, 98%, 99%, or 100% identity to the amino acid sequence of SEQ ID NO: 152, and/or one or more (e.g., 1, 2, 3, 4, 5, 6, 7, 8, 9, or 10) amino acid additions, deletions and/or substitutions in a framework region thereof. In some embodiments, the anti-PD-1xVEGF antibody comprises (A) a heavy chain (HC) comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 161, and (B) a light chain (LC) comprising an amino acid sequence having at least 80%, at least 85%, at least 90%, at least 95%, at least 96%, at least 97%, at least 98%, at least 99%, or 100% sequence identity to the amino acid sequence of SEQ ID NO: 152, wherein the HC comprises (i) the HCDR1, HCDR2, and HCDR3 sequences set forth in: SEQ ID NO: 135, 136, and 137, respectively, and (ii) the HCDR1, HCDR2, and HCDR3 sequences set forth in: SEQ ID NO: 110, 111, and 112, respectively, and the LC comprises the LCDR1, LCDR2, and LCDR3 sequences set forth in: SEQ ID NO: 138, 139, and 140, respectively. In some alternative embodiments, the anti-PD-1xVEGF antibody comprises (A′) a heavy chain (HC) comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 161, and (B′) a light chain (LC) comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 152, wherein the HC comprises (i) the HCDR1, HCDR2, and HCDR3 sequences set forth in: SEQ ID NO: 141, 142, and 143, respectively, and (ii) the HCDR1, HCDR2, and HCDR3 sequences set forth in: SEQ ID NO: 114, 115, and 116, respectively, and the LC comprises the LCDR1, LCDR2, and LCDR3 sequences set forth in: SEQ ID NO: 144, 145, and 140, respectively. In some preferred embodiments, the anti-PD-1xVEGF antibody comprises or consists of (i) a heavy chain sequence comprising or consisting of the amino acid sequence of SEQ ID NO: 161 or an amino acid sequence having at least 80%, 85%, 90%, 95%, 96%, 97%, 98%, 99%, or 100% identity to the amino acid sequence of SEQ ID NO: 161; and (ii) a light chain sequence comprising or consisting of the amino acid sequence of SEQ ID NO: 152 or an amino acid sequence having at least 80%, 85%, 90%, 95%, 96%, 97%, 98%, 99%, or 100% identity to the amino acid sequence of SEQ ID NO: 152.

In a third preferred embodiment, the anti-PD-1xVEGF antibody comprises (i) a heavy chain sequence comprising or consisting of the amino acid sequence of SEQ ID NO: 162 or an amino acid sequence having at least 80%, 85%, 90%, 95%, 96%, 97%, 98%, 99%, or 100% identity to the amino acid sequence of SEQ ID NO: 162, and/or one or more (e.g., 1, 2, 3, 4, 5, 6, 7, 8, 9, or 10) amino acid additions, deletions and/or substitutions in a framework region thereof, and (ii) a light chain sequence comprising or consisting of the amino acid sequence of SEQ ID NO: 152 or an amino acid sequence having at least 80%, 85%, 90%, 95%, 96%, 97%, 98%, 99%, or 100% identity to the amino acid sequence of SEQ ID NO: 152, and/or one or more (e.g., 1, 2, 3, 4, 5, 6, 7, 8, 9, or 10) amino acid additions, deletions and/or substitutions in a framework region thereof. In some embodiments, the anti-PD-1xVEGF antibody comprises (A) a heavy chain (HC) comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 162, and (B) a light chain (LC) comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 152, wherein the HC comprises (i) the HCDR1, HCDR2, and HCDR3 sequences set forth in: SEQ ID NO: 135, 136, and 137, respectively, and (ii) the HCDR1, HCDR2, and HCDR3 sequences set forth in: SEQ ID NO: 110, 111, and 113, respectively, and the LC comprises the LCDR1, LCDR2, and LCDR3 sequences set forth in: SEQ ID NO: 138, 139, and 140, respectively. In some alternative embodiments, the anti-PD-1xVEGF antibody comprises (A′) a heavy chain (HC) comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 162, and (B′) a light chain (LC) comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 152, wherein the HC comprises (i) the HCDR1, HCDR2, and HCDR3 sequences set forth in: SEQ ID NO: 141, 142, and 143, respectively, and (ii) the HCDR1, HCDR2, and HCDR3 sequences set forth in: SEQ ID NO: 114, 115, and 117, respectively, and the LC comprises the LCDR1, LCDR2, and LCDR3 sequences set forth in: SEQ ID NO: 144, 145, and 140, respectively. In some preferred embodiments, the anti-PD-1xVEGF antibody comprises or consists of (i) a heavy chain sequence comprising or consisting of the amino acid sequence of SEQ ID NO: 162 or an amino acid sequence having at least 80%, 85%, 90%, 95%, 96%, 97%, 98%, 99%, or 100% identity to the amino acid sequence of SEQ ID NO: 161; and (ii) a light chain sequence comprising or consisting of the amino acid sequence of SEQ ID NO: 152 or an amino acid sequence having at least 80%, 85%, 90%, 95%, 96%, 97%, 98%, 99%, or 100% identity to the amino acid sequence of SEQ ID NO: 152.

In some particularly preferred embodiments, the anti-PD-1xVEGF antibody comprises or consists of (i) a heavy chain sequence comprising or consisting of the amino acid sequence of SEQ ID NO: 160, 161, or 162; and (ii) a light chain sequence comprising or consisting of the amino acid sequence of SEQ ID NO: 152. In some particularly preferred embodiments, the anti-PD-1xVEGF antibody comprises or consists of (i) two heavy chains each comprising or consisting of the amino acid sequence of SEQ ID NO: 160, the amino acid sequence of 161, or the amino acid sequence of 162; and (ii) two light chains each comprising comprising or consisting of the amino acid sequence of SEQ ID NO: 152.

### C3. Anti-PD-L1xVEGFR Domain Antibodies

In some embodiments, the anti-PD-1/PD-L1xVEGF/VEGFR binding agent is a bispecific anti-PD-L1xVEGFR domain antibody, i.e., a bispecific antibody, wherein the first binding region of the bispecific antibody binds to PD-L1 and the second binding region comprises at least one VEGFR domain. Exemplary bispecific anti-PD-L1xVEGFR domain antibodies are the anti-PD-1/PD-L1xVEGF/VEGFR binding agents C3a and C3b provided in Table 1.

In some embodiments, the bispecific anti-PD-L1xVEGFR domain antibody may be a fusion protein comprising an anti-PD-L1 antibody or a fragment thereof and a VEGFR domain.

In some embodiments of the bispecific anti-PD-L1xVEGFR domain antibody, the bispecific fusion protein comprises any combination of one or more full-length antibodies, such as a human antibody, a humanized antibody, a chimeric antibody, a monoclonal antibody, or a polyclonal antibody, and/or one or more antibody fragments, such as a Fab, a Fab′, a F(ab′)2, an Fv, an scFv, a Fab′-SH, an sdAb, or a VHH; and at least one VEGFR domain. In some embodiments, the bispecific fusion protein comprises a full-length antibody and at least one VEGFR domain.

In some embodiments of the bispecific anti-PD-L1xVEGFR domain antibody, the anti-PD-L1 antibody or fragment thereof comprises a human antibody, a humanized antibody, or a chimeric antibody, preferably a humanized antibody. In some embodiments, the anti-PD-L1 antibody or fragment thereof is a monoclonal antibody, for example a monoclonal human antibody, a monoclonal humanized antibody, or a monoclonal chimeric antibody. Such antibodies or fragments thereof are fused to at least one, preferably at least two VEGFR domains, most preferably two VEGFR domains.

In some embodiments of the bispecific anti-PD-L1xVEGFR domain antibody, the anti-PD-L1 antibody or fragment thereof comprises an antibody fragment selected from a Fab, an Fab′, an F(ab′)2, an Fv, an scFv, an Fab′-SH, an sdAb, and a VHH, preferably a sdAb, most preferably a VHH. In some embodiments, the anti-PD-L1 antibody or fragment thereof is an anti-PD-L1 single domain antibody. In some embodiments, the anti-PD-L1 antibody or fragment thereof is an anti-PD-L1 VHH.

In some embodiments of the bispecific anti-PD-L1xVEGFR domain antibody, the VEGFR domain comprising protein comprises an Fc part and at least two VEGFR fused to the N-terminus thereof.

In some embodiments of the bispecific anti-PD-L1xVEGFR domain antibody, the bispecific fusion protein has monovalent binding to VEGF and/or to PD-L1. In some embodiments, the bispecific fusion protein has bivalent binding to VEGF and/or to PD-L1. In some preferred embodiments, the bispecific fusion protein has bivalent binding to VEGF and to PD-L1.

The bispecific fusion protein may be selected from SYN-2510 (ImmuneOnco/Instil Bio), HB0025 (Huabo Biopharm), and the anti-PD-1/PD-L1xVEGF/VEGFR binding agents C3a and C3b provided in Table 1.

In some embodiments of the bispecific anti-PD-L1xVEGFR domain antibody, the bispecific fusion protein comprises an anti-PD-L1 antibody, preferably a humanized anti-PD-L1 antibody, fused to a VEGFR domain. In some embodiments, the bispecific fusion protein comprises a full-length humanized antibody and two VEGFR domains.

The one or more VEGFR domains may be fused to the anti-PD-L1 antibody. In some embodiments, each VEGFR domain is terminally fused to the anti-PD-L1 antibody. In some embodiments, each VEGFR domain is fused to: (i) the C-terminus of a heavy chain; (ii) the N-terminus of a heavy chain; (iii) the C-terminus of a light chain; or (iv) the N-terminus of a light chain. In preferred embodiments, each VEGFR domain is fused to the N-terminus or the C-terminus of an anti-PD-L1 antibody, preferably the N-terminus of a heavy chain or the C-terminus of a heavy chain. The bispecific antibody preferably comprises a humanized anti-PD-L1 antibody and two VEGFR domains, wherein each heavy chain of the anti-PD-L1 antibody is fused to a VEGFR domain, preferably one VEGFR domain.

The VEGFR domain can be fused to the anti-PD-L1 antibody by a linker, e.g., a peptide linker, for example a glycine and serine-rich linker (GS-linker) or any suitable linker known in the art. An exemplary linker is GGSGGSGGSGGSGGS (SEQ ID NO: 168).

In some embodiments, the bispecific fusion protein comprises (a) an anti-PD-L1 antibody, preferably a humanized anti-PD-L1 antibody, comprising a heavy chain variable region, wherein the heavy chain variable region comprises: (i) a HCDR1 comprising or consisting of the amino acid sequence shown in SEQ ID NO: 127, or a variant thereof having up to three amino acid substitutions, additions or deletions; (ii) a HCDR2 comprising or consisting of the amino acid sequence shown in SEQ ID NO: 128, or a variant thereof having up to three amino acid substitutions, additions or deletions; and (iii) a HCDR3 comprising or consisting of the amino acid sequence shown in SEQ ID NO: 129, or a variant thereof having up to three amino acid substitutions, additions or deletions; and a light chain variable region, wherein the light chain variable region comprises: (i) a LCDR1 comprising or consisting of the amino acid sequence shown in SEQ ID NO: 130, or a variant thereof having up to three amino acid substitutions, additions or deletions; (ii) a LCDR2 comprising or consisting of the amino acid sequence shown in SEQ ID NO: 131, or a variant thereof having up to three amino acid substitutions, additions or deletions; and (iii) a LCDR3 comprising or consisting of the amino acid sequence shown in SEQ ID NO: 132, or a variant thereof having up to three amino acid substitutions, additions or deletions; and (b) a VEGFR domain comprising or consisting of the amino acid sequence shown in SEQ ID NO: 155, or an amino acid sequence having at least 80%, 85%, 90%, 95%, 96%, 97%, 98%, or 99% identity to SEQ ID NO: 155.

In some preferred embodiments, the anti-PD-L1xVEGFR domain antibody comprises or consists of (i) a heavy chain sequence comprising or consisting of the amino acid sequence of SEQ ID NO: 163 or an amino acid sequence having at least 80%, 85%, 90%, 95%, 96%, 97%, 98%, 99%, or 100% identity to the amino acid sequence of SEQ ID NO: 163, and/or one or more (e.g., 1, 2, 3, 4, 5, 6, 7, 8, 9, or 10) amino acid additions, deletions and/or substitutions in a framework region thereof, and (ii) a light chain sequence comprising or consisting of the amino acid sequence of SEQ ID NO: 164 or an amino acid sequence having at least 80%, 85%, 90%, 95%, 96%, 97%, 98%, 99%, or 100% identity to the amino acid sequence of SEQ ID NO: 164, and/or one or more (e.g., 1, 2, 3, 4, 5, 6, 7, 8, 9, or 10) amino acid additions, deletions and/or substitutions in a framework region thereof. In some embodiments, the anti-PD-L1xVEGFR domain antibody comprises (A) a heavy chain (HC) comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 163, and (B) a light chain (LC) comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 164, wherein the HC comprises the HCDR1, HCDR2, and HCDR3 sequences set forth in: SEQ ID NO: 127, 128, and 129, respectively, and the LC comprises the LCDR1, LCDR2, and LCDR3 sequences set forth in: SEQ ID NO: 130, 131, and 132, respectively. In some preferred embodiments, the anti-PD-L1xVEGFR domain antibody comprises or consists of (i) a heavy chain sequence comprising or consisting of the amino acid sequence of SEQ ID NO: 163 or an amino acid sequence having at least 80%, 85%, 90%, 95%, 96%, 97%, 98%, 99%, or 100% identity to the amino acid sequence of SEQ ID NO: 163; and (ii) a light chain sequence comprising or consisting of the amino acid sequence of SEQ ID NO: 164 or an amino acid sequence having at least 80%, 85%, 90%, 95%, 96%, 97%, 98%, 99%, or 100% identity to the amino acid sequence of SEQ ID NO: 164.

In some alternatively preferred embodiments, the anti-PD-L1xVEGFR domain antibody comprises or consist of (i) a heavy chain sequence comprising or consisting of the amino acid sequence of SEQ ID NO: 165 or an amino acid sequence having at least 80%, 85%, 90%, 95%, 96%, 97%, 98%, 99%, or 100% identity to the amino acid sequence of SEQ ID NO: 165, and/or one or more (e.g., 1, 2, 3, 4, 5, 6, 7, 8, 9, or 10) amino acid additions, deletions and/or substitutions in a framework region thereof, and (ii) a light chain sequence comprising or consisting of the amino acid sequence of SEQ ID NO: 164 or an amino acid sequence having at least 80%, 85%, 90%, 95%, 96%, 97%, 98%, 99%, or 100% identity to the amino acid sequence of SEQ ID NO: 164, and/or one or more (e.g., 1, 2, 3, 4, 5, 6, 7, 8, 9, or 10) amino acid additions, deletions and/or substitutions in a framework region thereof. In some embodiments, the anti-PD-L1xVEGFR domain antibody comprises or consists of (A) a heavy chain (HC) comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 165, and (B) a light chain (LC) comprising or consisting of an amino acid sequence having at least 80%, at least 85%, at least 90%, at least 95%, at least 96%, at least 97%, at least 98%, at least 99%, or 100% sequence identity to the amino acid sequence of SEQ ID NO: 164, wherein the HC comprises the HCDR1, HCDR2, and HCDR3 sequences set forth in: SEQ ID NO: 127, 128, and 129, respectively, and the LC comprises the LCDR1, LCDR2, and LCDR3 sequences set forth in: SEQ ID NO: 130, 131, and 132, respectively. In some preferred embodiments, the anti-PD-L1xVEGFR domain antibody comprises or consists of (i) a heavy chain sequence comprising or consisting of the amino acid sequence of SEQ ID NO: 165 or an amino acid sequence comprising or consisting of at least 80%, 85%, 90%, 95%, 96%, 97%, 98%, 99%, or 100% identity to the amino acid sequence of SEQ ID NO: 165; and (ii) a light chain sequence comprising or consisting of the amino acid sequence of SEQ ID NO: 164 or an amino acid sequence having at least 80%, 85%, 90%, 95%, 96%, 97%, 98%, 99%, or 100% identity to the amino acid sequence of SEQ ID NO: 164.

In some particularly preferred embodiments, the anti-PD-L1xVEGFR domain antibody comprises or consists of (i) a heavy chain sequence comprising or consisting of the amino acid sequence of SEQ ID NO: 163 or 165; and (ii) a light chain sequence comprising or consisting of the amino acid sequence of SEQ ID NO: 164. In some particularly preferred embodiments, the anti-PD-L1xVEGFR domain antibody comprises or consists of (i) two heavy chains each comprising or consisting of the amino acid sequence of SEQ ID NO: 163 or the amino acid sequence of SEQ 165; and (ii) two light chains each comprising or consisting of the amino acid sequence of SEQ ID NO: 164.

### C4. Anti-PD-1xVEGFR Antibodies

In some embodiments, the anti-PD-1/PD-L1xVEGF/VEGFR binding agent is a bispecific anti-PD-1xVEGF antibody, i.e., a bispecific antibody, wherein the first binding region of the bispecific antibody binds to PD-1 and the second binding region binds to VEGFR, preferably to VEGFR2. In some embodiments, the anti-PD-1/PD-L1xVEGF/VEGFR binding agent is an antibody or binding fragment thereof that specifically binds to both PD-1 and VEGFR, preferably VEGFR2. Exemplary bispecific anti-PD-1xVEGFR antibodies are the anti-PD-1/PD-L1xVEGF/VEGFR binding agents A5, A6, A7 and A8 provided in Table 1.

In some embodiments, the anti-PD-1xVEGFR antibody comprises one or more human variable domain framework scaffolds into which the CDRs have been inserted. For example, the VH region, the VL region, or both the VH and the VL region may comprise one or more human framework region amino acid sequences.

The anti-PD-1xVEGFR antibody or antigen-binding fragment thereof may comprise an IGHV3-7 human germline scaffold into which the corresponding HCDR sequences have been inserted. The anti-PD-1xVEGFR antibody or antigen-binding fragment thereof may comprise a VH region that comprises an IGHV3-7 human germline scaffold amino acid sequence into which a set of corresponding HCDR1, HCDR2 and HCDR3 amino acid sequences have been inserted.

The anti-PD-1xVEGFR antibody or antigen-binding fragment thereof may comprise an IGKV1-39 human germline scaffold into which the corresponding LCDR sequences have been inserted. The anti-PD-1xVEGFR antibody or antigen-binding fragment thereof may comprise a VL region that comprises an IGKV1-39 human germline scaffold amino acid sequence into which a set of corresponding LCDR1, LCDR2 and LCDR3 amino acid sequences have been inserted.

The anti-PD-1xVEGFR antibody or antigen-binding fragment thereof may comprise an IGHV3-7 human germline scaffold into which the corresponding HCDR sequences have been inserted and an IGKV1-39 human germline scaffold into which the corresponding LCDR sequences have been inserted. The anti-PD-1xVEGFR antibody or antigen-binding fragment thereof may comprise a VH region that comprises an IGHV3-7 human germline scaffold amino acid sequence into which a set of corresponding HCDR1, HCDR2 and HCDR3 amino acid sequences have been inserted and a VL region that comprises an IGKV1-39 human germline scaffold amino acid sequence into which a set of corresponding LCDR1, LCDR2 and LCDR3 amino acid sequences have been inserted. The HCDR1, HCDR2, HCDR3, LCDR1, LCDR2 and LCDR3 amino acid sequences may be the HCDR1, HCDR2, HCDR3, LCDR1, LCDR2 and LCDR3 amino acid sequences of any one of the following antibodies (anti-PD-1-antibody A5, anti-PD-1-antibody A6, anti-PD-1-antibody A7 and anti-PD-1-antibody A8).

In some embodiments, the anti-PD-1xVEGFR antibody or antigen-binding fragment thereof may comprise an immunoglobulin constant region. In some embodiments, the immunoglobulin constant region is IgG1, IgG2, IgG3, IgG4, IgAQ1 or IgA2. In additional embodiments, the immunoglobulin constant region is IgG1, IgG2, IgG3, IgG4, IgAQ1 or IgA2. The antibody molecule or antigen-binding fragment thereof may comprise an immunologically inert constant region. In some embodiments, an anti-PD1 and anti-VEGFR2 antibody or antigen-binding fragment thereof may comprise an immunoglobulin constant region comprising a wild-type human IgG1 constant region, a human IgG1 constant region comprising the amino acid substitutions L234A, L235A and G237A or a human IgG1 constant region comprising the amino acid substitutions L234A, L235A, G237A and P331S. In some embodiments, an anti-PD1 and anti-VEGFR2 antibody or antigen-binding fragment thereof may comprise an immunoglobulin constant region comprising a wild-type human IgG2 constant region or a wild-type human IgG4 constant region. In some embodiments, an anti-PD1 and anti-VEGFR2 antibody may comprise an immunoglobulin constant region comprising any one of the amino acid sequences in Table 6 of WO 2021/069670. The Fc region sequences in Table 6 begin at the CH1 domain. In some embodiments, an anti-PD1 and anti-VEGFR2 antibody may comprise an immunoglobulin constant region comprising an amino acid sequence of an Fc region of human IgG4, human IgG4(S228P), human IgG2, human IgG1, human IgG1-3M or human IgG1-4M. For example, the human IgG4(S228P) Fc region comprises the following substitution compared to the wild-type human IgG4 Fc region: S228P. For example, the human IgG1-3M Fc region comprises the following substitutions compared to the wild-type human IgG1 Fc region: L234A, L235A and G237A, while the human IgG1-4M Fc region comprises the following substitutions compared to the wild-type human IgG 1 Fc region: L234A, L235A, G237A and P331S. In some embodiments, a position of an amino acid residue in a constant region of an immunoglobulin molecule is numbered according to EU numbering of Kabat as specified herein. In some embodiments, an anti-PD1 and anti-VEGFR2 antibody may comprise an immunoglobulin constant region comprising any one of SEQ ID NOS: 25-31 of WO 2021/069670. In some embodiments, an anti-PD1 and anti-VEGFR2 antibody may comprise the six CDR amino acid sequences of any one of the following antibodies (anti-PD-1-antibody A5, anti-PD-1-antibody A6, anti-PD-1-antibody A7 and anti-PD-1-antibody A8) and any one of the Fc region amino acid sequences in Table 6 of WO 2021/069670. In some embodiments, an anti-PD1 and anti-VEGFR2 antibody may comprise an immunoglobulin heavy chain constant region comprising any one of the Fc region amino acid sequences in Table 6 of WO 2021/069670 and an immunoglobulin light chain constant region that is a kappa light chain constant region or a lambda light chain constant region.

### Example Administration Regimens

Preferably, the anti-PD-1/PD-L1xVEGF/VEGFR binding agent is administered in a suitable amount, i.e., the amount of anti-PD-1/PD-L1xVEGF/VEGFR binding agent administered, e.g., in each dose and/or treatment cycle, may slow the growth of cancer cells and/or decrease the viability of cancer cells, preferably kill cancer cells.

In some embodiments, the amount of anti-PD-1/PD-L1xVEGF/VEGFR binding agent administered, e.g., in each dose and/or in each treatment cycle, is about 1-1000 mg/kg body weight/day; about 0.1-500 mg/kg body weight/day; or about 0.01-100 mg/kg body weight/day.

The anti-PD-1/PD-L1xVEGF/VEGFR binding agent may be administered in any manner and by any route known in the art. In a preferred embodiment, the anti-PD-1/PD-L1xVEGF/VEGFR binding agent is administered systemically, such as parenterally, in particular intravenously or intraperitoneally.

The anti-PD-1/PD-L1xVEGF/VEGFR binding agent may be administered in the form of any suitable pharmaceutical composition as described herein. In a preferred embodiment, the anti-PD-1/PD-L1xVEGF/VEGFR binding agent is administered in the form of an injection or infusion.

The anti-PD-1/PD-L1xVEGF/VEGFR binding agent can be administered prior to, simultaneously with, or after administration of the ADC.

In one embodiment, the anti-PD-1/PD-L1xVEGF/VEGFR binding agent is administered prior to the administration of the ADC.

In one embodiment, the anti-PD-1/PD-L1xVEGF/VEGFR binding agent is administered after the administration of the ADC.

In one embodiment, the anti-PD-1/PD-L1xVGF/VEGFR binding agent is administered simultaneously with the ADC. For example, the anti-PD-1/PD-L1xVEGF/VEGFR binding agent and the ADC may be administered using a composition comprising both drugs.

Alternatively, the anti-PD-1/PD-L1xVEGF/VEGFR binding agent may be administered into one extremity of the subject, and the ADC may be administered into another extremity of the subject.

### Example Binding Agent Sequences

Table 1 below provides a listing of certain sequences referenced herein:

| | | |
|---|---|---|
| SEQ ID NO | Description | Sequence |

Anti-PD-1 antibody A1

| | | |
|---|---|---|
| 178 | HCDR1 (IMGT) | GFAFSSYD |
| 179 | HCDR2 (IMGT) | ISGGGRYT |
| 180 | HCDR3 (IMGT) | ANRYGEAWFAY |
| 181 | LCDRI (IMGT) | QDINTY |
| 182 | LCDR2 (IMGT) | RAN |
| 183 | LCDR3 (IMGT) | LQYDEFPLT |
| 184 | HCDR1 (Kabat) | SYDMS |
| 185 | HCDR2 (Kabat) | TISGGGRYTYYPDSVKG |
| 186 | HCDR3 (Kabat) | RYGEAWFAY |
| 187 | LCDR1 (Kabat) | RASQDINTYLS |
| 188 | LCDR2 (Kabat) | RANRLVS |
| 183 | LCDR3 (Kabat) | LQYDEFPLT |
| 189 | scFv fusion | EVQLVESGGGLVQPGGSLRLSCAASGFAFSSYDMSW |
| | | VRQAPGKGLDWVATISGGGRYTYYPDSVKGRFTISRD |
| | | NSKNNLYLQMNSLRAEDTALYYCANRYGEAWFAYW |
| | | GQGTLVTVSSGGGGSGGGGSGGGGSGGGGSDIQMTQ |
| | | SPSSMSASVGDRVTFTCRASQDINTYLSWFQQKPGKS |
| | | PKTLIYRANRLVSGVPSRFSGSGSGQDYTLTISSLQPED |
| | | MATYYCLQYDEFPLTFGAGTKLELKR |

Anti-PD-1 antibody A2

| | | |
|---|---|---|
| 110 | HCDR1 (Kabat) | RNFMG |
| 111 | HCDR2 (Kabat) | AIYTGTSRTYYADSVKG |
| 112 | HCDR3 a (Kabat) | DLRDGFWDTGVWNT |
| 113 | HCDR3 b (Kabat) | DLREGFWDTGVWNT |
| 114 | HCDR1 (IMGT) | GNIYNRNF |
| 115 | HCDR2 (IMGT) | IYTGTSRT |
| 116 | HCDR3 a (IMGT) | AADLRDGFWDTGVWNT |
| 117 | HCDR3 b (IMGT) | AADLREGFWDTGVWNT |
| 118 | VHH a | EVQLVESGGGLVQPGGSLRLSCAVSGNIYNRNFMGW |
| | | FRQAPGKGREGVSAIYTGTSRTYYADSVKGRFTISRD |
| | | NAKNTVYLQMNSLRPEDTAVYYCAADLRDGFWDTG |
| | | VWNTWGQGTLVTVSS |
| 119 | VHH b | EVQLVESGGGLVQPGGSLRLSCAVSGNIYNRNFMGW |
| | | FRQAPGKGLEGVSAIYTGTSRTYYADSVKGRFTISRDN |
| | | SKNTVYLQMNSLRAEDTAVYYCAADLREGFWDTGV |
| | | WNTWGQGTLVTVSS |

Anti-PD-LI antibody A3

| | | |
|---|---|---|
| 120 | HCDR1 (Kabat) | SYWMY |
| 121 | HCDR2 (Kabat) | SINSDSSSTYYRDSVKG |
| 122 | HCDR3 (Kabat) | DPGGYA |
| 123 | HCDR1 (IMGT) | GFTFSSYW |
| 124 | HCDR2 (IMGT) | INSDSSST |
| 125 | HCDR3 (IMGT) | AKDPGGYA |
| 126 | VHH | EVQLQESGGGLVQPGGSLRLSCAASGFTFSSYWMYW |
| | | LRQAPGKGLEWVSSINSDSSSTYYRDSVKGRFTISRDN |
| | | AKNTLYLQMNSLKSEDTAVYYCAKDPGGYAKGQGT |
| | | QVTVSS |

Anti-PD-L1 antibody A4

| | | |
|---|---|---|
| 127 | HCDR1 | GYAFTGYT |
| 128 | HCDR2 | FYPGSGTL |
| 129 | HCDR3 | ARHGTGTLMAMDY |
| 130 | LCDR1 | QSLANSYGNTY |
| 131 | LCDR2 | GIS |
| 132 | LCDR3 | LQGTHQPPT |
| 133 | VH | QVQLVQSGAEVKKPGASVKVSCKASGYAFTGYTIHW |
| | | VRQAPGQRLEWMGWFYPGSGTLKYSEKFQGRVTITR |
| | | DKSLSTAYMELSSLRSEDTAVYYCARHGTGTLMAMD |
| | | YWGQGTLVTVSS |
| 134 | VL | DVVMTQTPLSLSVTPGQPASISCKSSQSLANSYGNTYL |
| | | SWYLHKPGQSPQLLIYGISNRFSGVPDRFSGSGSGTDF |
| | | TLKISRVEAEDVGVYYCLQGTHQPPTFGQGTKLEIK |

Anti-PD-1 antibody A5

| | | |
|---|---|---|
| 330 | HCDR1 | GFTFSSYMMS |
| 331 | HCDR2 | TISGGGSNKYYVDSVKG |
| 332 | HCDR3 | QLYYFDY |
| 333 | LCDR1 | RASQESGIWLS |
| 334 | LCDR2 | TATSLAD |
| 335 | LCDR3 | QQVSVTPFT |
| 336 | VH | EVQLVESGGGLVQPGGSLRLSCAASGFTFSSYMMSW |
| | | VRQAPGKGLEWVATISGGGSNKYYVDSVKGRFTISRD |
| | | NAKNSLYLQMNSLRAEDTAVYYCARQLYYFDYWGQ |
| | | GTTVTVSS |
| 337 | VL | DIQMTQSPSSLSASVGDRVTITCRASQESGIWLSWYQQ |
| | | KPGKAPKLLIYTATSLADGVPSRFSGSGSGTDFTLTISS |
| | | LQPEDFATYYCQQVSVTPFTFGGGTKVEIK |

Anti-PD-1 antibody A6

| | | |
|---|---|---|
| 330 | HCDR1 | GFTFSSYMMS |
| 331 | HCR2 | TISGGGSNKYYVDSVKG |
| 332 | HCR3 | QLYYFDY |
| 333 | LCDR1 | RASQESGIWLS |
| 338 | LCDR2 | AASSLOS |
| 335 | LCDR3 | QQVSVTPFT |
| 336 | VH | EVQLVESGGGLVQPGGSLRLSCAASGFTFSSYMMSW |
| | | VRQAPGKGLEWVATISGGGSNKYYVDSVKGRFTISRD |
| | | NAKNSLYLQMNSLRAEDTAVYYCARQLYYFDYWGQ |
| | | GTTVTVSS |
| 339 | VL | DIQMTQSPSSLSASVGDRVTITCRASQESGIWLSWYQQ |
| | | KPGKAPKLLIYAASSLQSGVPSRFSGSGSGTDFTLTISS |
| | | LQPEDFATYYCQQVSVTPFTFGGGTKVEIK |

Anti-PD-1 antibody A7

| | | |
|---|---|---|
| 330 | HCDR1 | GFTFSSYMMS |
| 331 | HCDR2 | TISGGGSNKYYVDSVKG |
| 332 | HCDR3 | QLYYFDY |
| 333 | LCDR1 | RASQESGIWLS |
| 340 | LCDR2 | TASSLAD |
| 335 | LCDR3 | QQVSVTPFT |
| 336 | VH | EVQLVESGGGLVQPGGSLRLSCAASGFTFSSYMMSW |
| | | VRQAPGKGLEWVATISGGGSNKYYVDSVKGRFTISRD |
| | | NAKNSLYLQMNSLRAEDTAVYYCARQLYYFDYWGQ |
| | | GTTVTVSS |
| 341 | VL | DIQMTQSPSSLSASVGDRVTITCRASQESGIWLSWYQQ |
| | | KPGKAPKLLIYTASSLADGVPSRFSGSGSGTDFTLTISS |
| | | LQPEDFATYYCQQVSVTPFTFGGGTKVEIK |

Anti-PD-1 antibody A8

| | | |
|---|---|---|
| 330 | HCDR1 | GFTFSSYMMS |
| 331 | HCDR2 | TISGGGSNKYYVDSVKG |
| 332 | HCDR3 | QLYYFDY |
| 333 | LCDR1 | RASQESGIWLS |
| 342 | LCDR2 | AASSLAD |
| 335 | LCDR3 | QQVSVTPFT |
| 336 | VH | EVQLVESGGGLVQPGGSLRLSCAASGFTFSSYMMSW |
| | | VRQAPGKGLEWVATISGGGSNKYYVDSVKGRFTISRD |
| | | NAKNSLYLQMNSLRAEDTAVYYCARQLYYFDYWGQ |
| | | GTTVTVSS |
| 343 | VL | DIQMTQSPSSLSASVGDRVTITCRASQESGIWLSWYQQ |
| | | KPGKAPKLLIYAASSLADGVPSRFSGSGSGTDFTLTISS |
| | | LQPEDFATYYCQQVSVTPFTFGGGTKVEIK |

Anti-VEGF antibody

| | | |
|---|---|---|
| 135 | HCDR1 (Kabat) | NYGMN |
| 136 | HCDR2 (Kabat) | WINTYTGEPTYAADFKR |
| 137 | HCDR3 (Kabat) | YPHYYGSSHWYFDV |
| 138 | LCDRI (Kabat) | SASQDISNYLN |
| 139 | LCDR2 (Kabat) | FTSSLHS |
| 140 | LCDR3 (Kabat) | QQYSTVPWT |
| 141 | HCDR1 (IMGT) | GYTFTNYG |
| 142 | HCDR2 (IMGT) | INTYTGEP |
| 143 | HCDR3 (IMGT) | AKYPHYYGSSHWYFDV |
| 144 | LCDRI (IMGT) | QDISNY |
| 145 | LCDR2 (IMGT) | FTS |
| 140 | LCDR3 (IMGT) | QQYSTVPWT |
| 146 | VH | EVQLVESGGGLVQPGGSLRLSCAASGYTFTNYGMNW |
| | | VRQAPGKGLEWVGWINTYTGEPTYAADFKRRFTFSL |
| | | DTSKSTAYLQMNSLRAEDTAVYYCAKYPHYYGSSHW |
| | | YFDVWGQGTLVTVSS |
| 147 | VL | DIQMTQSPSSLSASVGDRVTITCSASQDISNYLNWYQQ |
| | | KPGKAPKVLIYFTSSLHSGVPSRFSGSGSGTDFTLTISS |
| | | LQPEDFATYYCQQYSTVPWTFGQGTKVEIK |
| 148 | CH1 | ASTKGPSVFPLAPSSKSTSGGTAALGCLVKDYFPEPVT |
| | | VSWNSGALTSGVHTFPAVLQSSGLYSLSSVVTVPSSSL |
| | | GTQTYICNVNHKPSNTKVDKKVEPKSC |
| 149 | Fc | DKTHTCPPCPAPEAAGGPSVFLFPPKPKDTLMISRTPE |
| | | VTCVVVDVSHEDPEVKFNWYVDGVEVHNAKTKPRE |
| | | EQYNSTYRVVSVLTVLHQDWLNGKEYKCKVSNKALP |
| | | APIEKTISKAKGQPREPQVYTLPPSRDELTKNQVSLTC |
| | | LVKGFYPSDIAVEWESNGQPENNYKTTPPVLDSDGSF |
| | | FLYSKLTVDKSRWQQGNVFSCSVMHEALHNHYTQKS |
| | | LSLSPG |
| 150 | CL | RTVAAPSVFIFPPSDEQLKSGTASVVCLLNNFYPREAK |
| | | VQWKVDNALQSGNSQESVTEQDSKDSTYSLSSTLTLS |
| | | KADYEKHKVYACEVTHQGLSSPVTKSFNRGEC |
| 151 | Heavy chain | EVQLVESGGGLVQPGGSLRLSCAASGYTFTNYGMNW |
| | | VRQAPGKGLEWVGWINTYTGEPTYAADFKRRFTFSL |
| | | DTSKSTAYLQMNSLRAEDTAVYYCAKYPHYYGSSHW |
| | | YFDVWGQGTLVTVSSASTKGPSVFPLAPSSKSTSGGT |
| | | AALGCLVKDYFPEPVTVSWNSGALTSGVHTFPAVLQS |
| | | SGLYSLSSVVTVPSSSLGTQTYICNVNHKPSNTKVDKK |
| | | VEPKSCDKTHTCPPCPAPEAAGGPSVFLFPPKPKDTLM |
| | | ISRTPEVTCVVVDVSHEDPEVKFNWYVDGVEVHNAK |
| | | TKPREEQYNSTYRVVSVLTVLHQDWLNGKEYKCKVS |
| | | NKALPAPIEKTISKAKGQPREPQVYTLPPSRDELTKNQ |
| | | VSLTCLVKGFYPSDIAVEWESNGQPENNYKTTPPVLD |
| | | SDGSFFLYSKLTVDKSRWQQGNVFSCSVMHEALHNH |
| | | YTQKSLSLSPG |
| 152 | Light chain | DIQMTQSPSSLSASVGDRVTITCSASQDISNYLNWYQQ |
| | | KPGKAPKVLIYFTSSLHSGVPSRFSGSGSGTDFTLTISS |
| | | LQPEDFATYYCQQYSTVPWTFGQGTKVEIKRTVAAPS |
| | | VFIFPPSDEQLKSGTASVVCLLNNFYPREAKVQWKVD |
| | | NALQSGNSQESVTEQDSKDSTYSLSSTLTLSKADYEK |
| | | HKVYACEVTHQGLSSPVTKSFNRGEC |

VEGFR domains

| | | |
|---|---|---|
| 153 | Domain 1 | SDTGRPFVEMYSEIPEIIHMTEGRELVIPCRVTSPNITVT |
| | | LKKFPLDTLIPDGKRIIWDSRKGFIISNATYKEIGLLTCE |
| | | ATVNGHLYKTNYLTHRQTNTII |
| 154 | Domain 2 | DVVLSPSHGIELSVGEKLVLNCTARTELNVGIDENWE |
| | | YPSSKHQHKKLVNRDLKTQSGSEMKKFLSTLTIDGVT |
| | | RSDQGLYTCAASSGLMTKKNSTFVRVHEK |
| 155 | Domain 3 | SDTGRPFVEMYSEIPEIIHMTEGRELVIPCRVTSPNITVT |
| | | LKKFPLDTLIPDGKRIIWDSRKGFIISNATYKEIGLLTCE |
| | | ATVNGHLYKTNYLTHRQTNT |
| 156 | Domain 4 | SDTGRPFVEMYSEIPEIIHMTEGRELVIPCRVTSPNITVT |
| | | LKKFPLDTLIPDGKRIIWDSRKGFIISNATYKEIGLLTCE |
| | | ATVNGHLYKTNYLTHRQTNTIIDVVLSPSHGIELSVGE |
| | | KLVLNCTARTELNVGIDENWEYPSSKHQHKKLVNRD |
| | | LKTQSGSEMKKFLSTLTIDGVTRSDQGLYTCAASSGL |
| | | MTKKNSTFVR VHEK |
| 157 | Domain 5 | SDTGRPFVEMYSEIPEIIHMTEGRELVIPCRVTSPNITVT |
| | | LKKFPLDTLIPDGKRIIWDSRKGFIISNATYKEIGLLTCE |
| | | ATVNGHLYKTNYLTHRQTNTIIDVVLSPSHGIELSVGE |
| | | KLVLNCTARTELNVGIDENWEYPSSKHQHKKLVNRD |
| | | LKTQSGSEMKKFLSTLTIDGVTRSDQGLYTCAASSGL |
| | | MTKKNSTFVRVHEKDKTHTCPPCPAPELLGGPSVFLFP |
| | | PKPKDTLMISRTPEVTCVVVDVSHEDPEVKFNWYVD |
| | | GVEVHNAKTKPREEQYNSTYRVVSVLTVLHQDWLNG |
| | | KEYKCKVSNKALPAPIEKTISKAKGQPREPQVYTLPPS |
| | | RDELTKNQVSLTCLVKGFYPSDIAVEWESNGQPENNY |
| | | KTTPPVLDSDGSFFLYSKLTVDKSRWQQGNVFSCSVM |
| | | HEALHNHYTQK SLSLSPGK |

Bispecific antibody PD-L1 x VEGF B1 (C1)

| | | |
|---|---|---|
| 158 | HC VHH fusion | EVQLVESGGGLVQPGGSLRLSCAASGYTFTNYGMNW |
| | | VRQAPGKGLEWVGWINTYTGEPTY AADFKRRFTFSL |
| | | DTSKSTAYLQMNSLRAEDTAVYYCAKYPHYYGSSHW |
| | | YFDVWGQGTLVTVSSASTKGPSVFPLAPSSKSTSGGT |
| | | AALGCLVKDYFPEPVTVSWNSGALTSGVHTFPAVLQS |
| | | SGLYSLSSVVTVPSSSLGTQTYICNVNHKPSNTKVDKK |
| | | VEPKSCDKTHTCPPCPAPEAAGGPSVFLFPPKPKDTLM |
| | | ISRTPEVTCVVVDVSHEDPEVKFNWYVDGVEVHNAK |
| | | TKPREEQYNSTYRVVSVLTVLHQDWLNGKEYKCKVS |
| | | NKALPAPIEKTISKAKGQPREPQVYTLPPSRDELTKNQ |
| | | VSLTCLVKGFYPSDIAVEWESNGQPENNYKTTPPVLD |
| | | SDGSFFLYSKLTVDKSRWQQGNVFSCSVMHEALHNH |
| | | YTQKSLSLSPGGGGGSGGGGSGEVQLQESGGGLVQPG |
| | | GSLRLSCAASGFTFSSYWMYWLRQAPGKGLEWVSSI |
| | | NSDSSSTYYRDSVKGRFTISRDNAKNTLYLQMNSLKS |
| | | EDTAVYYCAKDPGGYAKGQGTQVTVSS |
| 152 | LC | DIQMTQSPSSLSASVGDRVTITCSASQDISNYLNWYQQ |
| | | KPGKAPKVLIYFTSSLHSGVPSRFSGSGSGTDFTLTISS |
| | | LQPEDFATYYCQQYSTVPWTFGQGTKVEIKRTVAAPS |
| | | VFIFPPSDEQLKSGTASVVCLLNNFYPREAKVQWKVD |
| | | NALQSGNSQESVTEQDSKDSTYSLSSTLTLSKADYEK |
| | | HKVYACEVTHQGLSSPVTKSFNRGEC |

Bispecific antibody PD-1 x VEGF B2 (C2a)
(referred to herein as “Compound 13”)

| | | |
|---|---|---|
| 159 | HC scFv fusion | EVQLVESGGGLVQPGGSLRLSCAASGYTFTNYGMNW |
| | | VRQAPGKGLEWVGWINTYTGEPTYAADFKRRFTFSL |
| | | DTSKSTAYLQMNSLRAEDTAVYYCAKYPHYYGSSHW |
| | | YFDVWGQGTLVTVSSASTKGPSVFPLAPSSKSTSGGT |
| | | AALGCLVKDYFPEPVTVSWNSGALTSGVHTFPAVLQS |
| | | SGLYSLSSVVTVPSSSLGTQTYICNVNHKPSNTKVDKK |
| | | VEPKSCDKTHTCPPCPAPEAAGGPSVFLFPPKPKDTLM |
| | | ISRTPEVTCVVVDVSHEDPEVKFNWYVDGVEVHNAK |
| | | TKPREEQYNSTYRVVSVLTVLHQDWLNGKEYKCKVS |
| | | NKALPAPIEKTISKAKGQPREPQVYTLPPSRDELTKNQ |
| | | VSLTCLVKGFYPSDIAVEWESNGQPENNYKTTPPVLD |
| | | SDGSFFLYSKLTVDKSRWQQGNVFSCSVMHEALHNH |
| | | YTQKSLSLSPGKGGGGSGGGGSGGGGSGGGGSEVQL |
| | | VESGGGLVQPGGSLRLSCAASGFAFSSYDMSWVRQA |
| | | PGKGLDWVATISGGGRYTYYPDSVKGRFTISRDNSKN |
| | | NLYLQMNSLRAEDTALYYCANRYGEAWFAYWGQGT |
| | | LVTVSSGGGGSGGGGSGGGGSGGGGSDIQMTQSPSS |
| | | MSASVGDRVTFTCRASQDINTYLSWFQQKPGKSPKTL |
| | | IYRANRLVSGVPSRFSGSGSGQDYTLTISSLQPEDMAT |
| | | YYCLQYDEFPLTFGAGTKLELKR |
| 152 | LC | DIQMTQSPSSLSASVGDRVTITCSASQDISNYLNWYQQ |
| | | KPGKAPKVLIYFTSSLHSGVPSRFSGSGSGTDFTLTISS |
| | | LQPEDFATYYCQQYSTVPWTFGQGTKVEIKRTVAAPS |
| | | VFIFPPSDEQLKSGTASVVCLLNNFYPREAKVQWKVD |
| | | NALQSGNSQESVTEQDSKDSTYSLSSTLTLSKADYEK |
| | | HKVYACEVTHQGLSSPVTKSFNRGEC |

Bispecific antibody PD-1 x VEGF B3 (C2b)

| | | |
|---|---|---|
| 160 | HC VHH fusion | EVQLVESGGGLVQPGGSLRLSCAASGYTFTNYGMNW |
| | | VRQAPGKGLEWVGWINTYTGEPTYAADFKRRFTFSL |
| | | DTSKSTAYLQMNSLRAEDTAVYYCAKYPHYYGSSHW |
| | | YFDVWGQGTLVTVSSASTKGPSVFPLAPSSKSTSGGT |
| | | AALGCLVKDYFPEPVTVSWNSGALTSGVHTFPAVLQS |
| | | SGLYSLSSVVTVPSSSLGTQTYICNVNHKPSNTKVDKK |
| | | VEPKSCDKTHTCPPCPAPEAAGGPSVFLFPPKPKDTLM |
| | | ISRTPEVTCVVVDVSHEDPEVKFNWYVDGVEVHNAK |
| | | TKPREEQYNSTYRVVSVLTVLHQDWLNGKEYKCKVS |
| | | NKALPAPIEKTISKAKGQPREPQVYTLPPSRDELTKNQ |
| | | VSLTCLVKGFYPSDIAVEWESNGQPENNYKTTPPVLD |
| | | SDGSFFLYSKLTVDKSRWQQGNVFSCSVMHEALHNH |
| | | YTQKSLSLSPGKGGGGSGGGGSGGGGSGGGGSEVQL |
| | | VESGGGLVQPGGSLRLSCAVSGNIYNRNFMGWFRQA |
| | | PGKGREGVSAIYTGTSRTYYADSVKGRFTISRDNAKN |
| | | TVYLQMNSLRPEDTAVYYCAADLRDGFWDTGVWNT |
| | | WGQGTLVTVSSGGGGSGGGGSGGGGSGGGGSEVQL |
| | | VESGGGLVQPGGSLRLSCAVSGNIYNRNFMGWFRQA |
| | | PGKGREGVSAIYTGTSRTYYADSVKGRFTISRDNAKN |
| | | TVYLQMNSLRPEDTAVYYCAADLRDGFWDTGVWNT |
| | | WGQGTLVTVSS |
| 152 | LC | DIQMTQSPSSLSASVGDRVTITCSASQDISNYLNWYQQ |
| | | KPGKAPKVLIYFTSSLHSGVPSRFSGSGSGTDFTLTISS |
| | | LQPEDFATYYCQQYSTVPWTFGQGTKVEIKRTVAAPS |
| | | VFIFPPSDEQLKSGTASVVCLLNNFYPREAKVQWKVD |
| | | NALQSGNSQESVTEQDSKDSTYSLSSTLTLSKADYEK |
| | | HKVYACEVTHQGLSSPVTKSFNRGEC |

Bispecific antibody PD-1 x VEGF B4 (C2c)

| | | |
|---|---|---|
| 161 | HC VHH fusion | EVQLVESGGGLVQPGGSLRLSCAASGYTFTNYGMNW |
| | | VRQAPGKGLEWVGWINTYTGEPTYAADFKRRFTFSL |
| | | DTSKSTAYLQMNSLRAEDTAVYYCAKYPHYYGSSHW |
| | | YFDVWGQGTLVTVSSASTKGPSVFPLAPSSKSTSGGT |
| | | AALGCLVKDYFPEPVTVSWNSGALTSGVHTFPAVLQS |
| | | SGLYSLSSVVTVPSSSLGTQTYICNVNHKPSNTKVDKK |
| | | VEPKSCDKTHTCPPCPAPEAAGGPSVFLFPPKPKDTLM |
| | | ISRTPEVTCVVVDVSHEDPEVKFNWYVDGVEVHNAK |
| | | TKPREEQYNSTYRVVSVLTVLHQDWLNGKEYKCKVS |
| | | NKALPAPIEKTISKAKGQPREPQVYTLPPSRDELTKNQ |
| | | VSLTCLVKGFYPSDIAVEWESNGQPENNYKTTPPVLD |
| | | SDGSFFLYSKLTVDKSRWQQGNVFSCSVMHEALHNH |
| | | YTQKSLSLSPGGGGGSGGGGSGGGGSGGGGSEVQLV |
| | | ESGGGLVQPGGSLRLSCAVSGNIYNRNFMGWFRQAP |
| | | GKGREGVSAIYTGTSRTYYADSVKGRFTISRDNAKNT |
| | | VYLQMNSLRPEDTAVYYCAADLRDGFWDTGVWNT |
| | | WGQGTLVTVSS |
| 152 | LC | DIQMTQSPSSLSASVGDRVTITCSASQDISNYLNWYQQ |
| | | KPGKAPKVLIYFTSSLHSGVPSRFSGSGSGTDFTLTISS |
| | | LQPEDFATYYCQQYSTVPWTFGQGTKVEIKRTVAAPS |
| | | VFIFPPSDEQLKSGTASVVCLLNNFYPREAKVQWKVD |
| | | NALQSGNSQESVTEQDSKDSTYSLSSTLTLSKADYEK |
| | | HKVYACEVTHQGLSSPVTKSFNRGEC |

Bispecific antibody PD-1 x VEGF B5 (C2d)

| | | |
|---|---|---|
| 162 | HC VHH fusion | EVQLVESGGGLVQPGGSLRLSCAASGYTFTNYGMNW |
| | | VRQAPGKGLEWVGWINTYTGEPTYAADFKRRFTFSL |
| | | DTSKSTAYLQMNSLRAEDTAVYYCAKYPHYYGSSHW |
| | | YFDVWGQGTLVTVSSASTKGPSVFPLAPSSKSTSGGT |
| | | AALGCLVKDYFPEPVTVSWNSGALTSGVHTFPAVLQS |
| | | SGLYSLSSVVTVPSSSLGTQTYICNVNHKPSNTKVDKK |
| | | VEPKSCDKTHTCPPCPAPEAAGGPSVFLFPPKPKDTLM |
| | | ISRTPEVTCVVVDVSHEDPEVKFNWYVDGVEVHNAK |
| | | TKPREEQYNSTYRVVSVLTVLHQDWLNGKEYKCKVS |
| | | NKALPAPIEKTISKAKGQPREPQVYTLPPSRDELTKNQ |
| | | VSLTCLVKGFYPSDIAVEWESNGQPENNYKTTPPVLD |
| | | SDGSFFLYSKLTVDKSRWQQGNVFSCSVMHEALHNH |
| | | YTQKSLSLSPGGGGGSGGGGSGGGGSGGGGSEVQLV |
| | | ESGGGLVQPGGSLRLSCAVSGNIYNRNFMGWFRQAP |
| | | GKGLEGVSAIYTGTSRTYYADSVKGRFTISRDNSKNT |
| | | VYLQMNSLRAEDTAVYYCAADLREGFWDTGVWNT |
| | | WGQGTLVTVSS |
| 152 | LC | DIQMTQSPSSLSASVGDRVTITCSASQDISNYLNWYQQ |
| | | KPGKAPKVLIYFTSSLHSGVPSRFSGSGSGTDFTLTISS |
| | | LQPEDFATYYCQQYSTVPWTFGQGTKVEIKRTVAAPS |
| | | VFIFPPSDEQLKSGTASVVCLLNNFYPREAKVQWKVD |
| | | NALQSGNSQESVTEQDSKDSTYSLSSTLTLSKADYEK |
| | | HKVYACEVTHQGLSSPVTKSFNRGEC |

Bispecific fusion protein PD-L1 x VEGFR B6 (C3a)

| | | |
|---|---|---|
| 163 | HC fused to | SDTGRPFVEMYSEIPEIIHMTEGRELVIPCRVTSPNITVT |
| | VEGFR domain | LKKFPLDTLIPDGKRIIWDSRKGFIISNATYKEIGLLTCE |
| | (N terminus) | ATVNGHLYKTNYLTHRQTNTGGSGGSGGSGGSGGSQ |
| | | VQLVQSGAEVKKPGASVKVSCKASGYAFTGYTIHWV |
| | | RQAPGQRLEWMGWFYPGSGTLKYSEKFQGRVTITRD |
| | | KSLSTAYMELSSLRSEDTAVYYCARHGTGTLMAMDY |
| | | WGQGTLVTVSSASTKGPSVFPLAPSSKSTSGGTAALG |
| | | CLVKDYFPEPVTVSWNSGALTSGVHTFPAVLQSSGLY |
| | | SLSSVVTVPSSSLGTQTYICNVNHKPSNTKVDKRVEPK |
| | | SCDKTHTCPPCPAPEAAGGPSVFLFPPKPKDTLMISRT |
| | | PEVTCVVVDVSHEDPEVKFNWYVDGVEVHNAKTKPR |
| | | EEQYNSTYRVVSVLTVLHQDWLNGKEYKCKVSNKAL |
| | | PAPIEKTISKAKGQPREPQVYTLPPSREEMTKNQVSLT |
| | | CLVKGFYPSDIAVEWESNGQPENNYKTTPPVLDSDGS |
| | | FFLYSKLTVDKSRWQQGNVFSCSVMHEALHNHYTQK |
| | | SLSLSPG |
| 164 | FP LC | DVVMTQTPLSLSVTPGQPASISCKSSQSLANSYGNTYL |
| | | SWYLHKPGQSPQLLIYGISNRFSGVPDRFSGSGSGTDF |
| | | TLKISRVEAEDVGVYYCLQGTHQPPTFGQGTKLEIKRT |
| | | VAAPSVFIFPPSDEQLKSGTASVVCLLNNFYPREAKVQ |
| | | WKVDNALQSGNSQESVTEQDSKDSTYSLSSTLTLSKA |
| | | DYEKHKVYACEVTHQGLSSPVTKSFNRGEC |

Bispecific fusion protein PD-L1 x VEGFR B7 (C3b)

| | | |
|---|---|---|
| 165 | HC fused to | QVQLVQSGAEVKKPGASVKVSCKASGYAFTGYTIHW |
| | VEGFR domain | VRQAPGQRLEWMGWFYPGSGTLKYSEKFQGRVTITR |
| | (C terminus) | DKSLSTAYMELSSLRSEDTAVYYCARHGTGTLMAMD |
| | | YWGQGTLVTVSSASTKGPSVFPLAPSSKSTSGGTAAL |
| | | GCLVKDYFPEPVTVSWNSGALTSGVHTFPAVLQSSGL |
| | | YSLSSVVTVPSSSLGTQTYICNVNHKPSNTKVDKRVEP |
| | | KSCDKTHTCPPCPAPEAAGGPSVFLFPPKPKDTLMISR |
| | | TPEVTCVVVDVSHEDPEVKFNWYVDGVEVHNAKTKP |
| | | REEQYNSTYRVVSVLTVLHQDWLNGKEYKCKVSNK |
| | | ALPAPIEKTISKAKGQPREPQVYTLPPSREEMTKNQVS |
| | | LTCLVKGFYPSDIAVEWESNGQPENNYKTTPPVLDSD |
| | | GSFFLYSKLTVDKSRWQQGNVFSCSVMHEALHNHYT |
| | | QKSLSLSPGGGSGGSGGSGGSGGSSDTGRPFVEMYSEI |
| | | PEIIHMTEGRELVIPCRVTSPNITVTLKKFPLDTLIPDGK |
| | | RIIWDSRKGFIISNATYKEIGLLTCEATVNGHLYKTNY |
| | | LTHRQTNT |
| 164 | FP LC | DVVMTQTPLSLSVTPGQPASISCKSSQSLANSYGNTYL |
| | | SWYLHKPGQSPQLLIYGISNRFSGVPDRFSGSGSGTDF |
| | | TLKISRVEAEDVGVYYCLQGTHQPPTFGQGTKLEIKRT |
| | | VAAPSVFIFPPSDEQLKSGTASVVCLLNNFYPREAKVQ |
| | | WKVDNALQSGNSQESVTEQDSKDSTYSLSSTLTLSKA |
| | | DYEKHKVYACEVTHQGLSSPVTKSFNRGEC |

Linker for bispecific antibodies

| | | |
|---|---|---|
| 166 | Linker a | GGGGSGGGGSG |
| 167 | Linker b | GGGGSGGGGSGGGGSGGGGS |
| 168 | Linker c | GGSGGSGGSGGSGGS |

PD-1, PD-L1, VEGF, VEGFR

| | | |
|---|---|---|
| 169 | PD-1 | MQIPQAPWPVVWAVLQLGWRPGWFLDSPDRPWNPP |
| | | TFSPALLVVTEGDNATFTCSFSNTSESFVLNWYRMSPS |
| | | NQTDKLAAFPEDRSQPGQDCRFRVTQLPNGRDFHMS |
| | | VVRARRNDSGTYLCGAISLAPKAQIKESLRAELRVTER |
| | | RAEVPTAHPSPSPRPAGQFQTLVVGVVGGLLGSLVLL |
| | | VWVLAVICSRAARGTIGARRTGQPLKEDPSAVPVFSV |
| | | DYGELDFQWREKTPEPPVPCVPEQTEYATIVFPSGMG |
| | | TSSPARRGSADGPRSAQPLRPEDGHCSWPL |
| 170 | PD-1 isoform 1 | MQIPQAPWPVVWAVLQLGWRPGWFLDSPDRPWNPP |
| | | TFFPALLVVTEGDNATFTCSFSNTSESFVLNWYRMSPS |
| | | NQTDKLAAFPEDRSQPGQDCRFRVTQLPNGRDFHMS |
| | | VVRARRNDSGTYLCGAISLAPKAQIKESLRAELRVTER |
| | | RAEVPTAHPSPSPRPAGQFQTLVVGVVGGLLGSLVLL |
| | | VWVLAVICSRAARGTIGARRTGQPLKEDPSAVPVFSV |
| | | DYGELDFQWREKTPEPPVPCVPEQTEYATIVFPSGMG |
| | | TSSPARRGSADGPRSAQPLRPEDGHCSWPL |
| 171 | PD-1 isoform 2 | MQIPQAPWPVVWAVLQLGWRPGWFLDSPDRPWNPP |
| | | TFSPALLVVTEGDNATFTCSFSNTSESFVLNWYRMSPS |
| | | NQTDKLAAFPEDRSQPGQDCRFRVTQLPNGRDFHMS |
| | | VVRARRNDSGTYLCGAISLAPKAQIKESLRAELRVTER |
| | | RAEVPTAHPSPSPRSAGQFQTLVVGVVGGLLGSLVLL |
| | | VWVLAVICSRAARGTIGARRTGQPLKEDPSAVPVFSV |
| | | DYGELDFQWREKTPEPPVPCVPEQTEYATIVFPSGMG |
| | | TSSPARRGSADGPRSAQPLRPEDGHCSWPL |
| 172 | PD-L1 | MRIFAVFIFMTYWHLLNAFTVTVPKDLYVVEYGSNM |
| | | TIECKFPVEKQLDLAALIVYWEMEDKNIIQFVHGEEDL |
| | | KVQHSSYRQRARLLKDQLSLGNAALQITDVKLQDAG |
| | | VYRCMISYGGADYKRITVKVNAPYNKINQRILVVDPV |
| | | TSEHELTCQAEGYPKAEVIWTSSDHQVLSGKTTTTNS |
| | | KREEKLFNVTSTLRINTTTNEIFYCTFRRLDPEENHTAE |
| | | LVIPELPLAHPPNERTHLVILGAILLCLGVALTFIFRLRK |
| | | GRMMDVKKCGIQDTNSKKQSDTHLEET |
| 173 | PD-L1 isoform 1 | MRIFAVFIFMTYWHLLNAPYNKINQRILVVDPVTSEHE |
| | | LTCQAEGYPKAEVIWTSSDHQVLSGKTTTTNSKREEK |
| | | LFNVTSTLRINTTTNEIFYCTFRRLDPEENHTAELVIPEL |
| | | PLAHPPNERTHLVILGAILLCLGVALTFIFRLRKGRMM |
| | | DVKKCGIQDTNSKKQSDTHLEET |
| 174 | PD-L1 isoform | MRIFAVFIFMTYWHLLNAPYNKINQRILVVDPVTSEHE |
| | 2 | LTCQAEGYPKAEVIWTSSDHQVLSGKTTTTNSKREEK |
| | | LFNVTSTLRINTTTNEIFYCTFRRLDPEENHTAELVIPEL |
| | | PLAHPPNERTHLVILGAILLCLGVALTFIFRLRKGRMM |
| | | DVKKCGIQDTNSKKQSDTHLEET |
| 175 | PD-L1 isoform | MRIFAVFIFMTYWHLLNAFTVTVPKDLYVVEYGSNM |
| | 3 | TIECKFPVEKQLDLAALIVYWEMEDKNIIQFVHGEEDL |
| | | KVQHSSYRQRARLLKDQLSLGNAALQITDVKLQDAG |
| | | VYRCMISYGGADYKRITVKVNAPYNKINQRILVVDPV |
| | | TSEHELTCQAEGYPKAEVIWTSSDHQVLSGD |
| 176 | VEGF | MNFLLSWVHWSLALLLYLHHAK |
| 177 | VEGF-A | MTDRQTDTAPSPSYHLLPGRRRTVDAAASRGQGPEPA |
| | | PGGGVEGVGARGVALKLFVQLLGCSRFGGAVVRAGE |
| | | AEPSGAARSASSGREEPQPEEGEEEEEKEEERGPQWRL |
| | | GARKPGSWTGEAAVCADSAPAARAPQALARASGRGG |
| | | RVARRGAEESGPPHSPSRRGSASRAGPGRASETMNFL |
| | | LSWVHWSLALLLYLHHAKWSQAAPMAEGGGQNHHE |
| | | VVKFMDVYQRSYCHPIETLVDIFQEYPDEIEYIFKPSCV |
| | | PLMRCGGCCNDEGLECVPTEESNITMQIMRIKPHQGQ |
| | | HIGEMSFLQHNKCECRPKKDRARQEKKSVRGKGKGQ |
| | | KRKRKKSRYKSWSVPCGPCSERRKHLFVQDPQTCKC |
| | | SCKNTDSRCKARQLELNERTCRCDKPRR |
| 344 | Human VEGFR2 | MQSKVLLAVALWLCVETRAASVGLPSVSLDLPRLSIQ |
| | domain | KDILTIKANTTLQITCRGQRDLDWLWPNNQSGSEQRV |
| | | EVTECSDGLFCKTLTIPKVIGNDTGAYKCFYRETDLAS |
| | | VIYVYVQDYRSPFIASVSDQHGVVYITENKNKTVVIPC |
| | | LGSISNLNVSLCARYPEKRFVPDGNRISWDSKKGFTIPS |
| | | YMISYAGMVFCEAKINDESYQSIMYIVVVVGYRIYDV |
| | | VLSPSHGIELSVGEKLVLNCTARTELNVGIDENWEYPS |
| | | SKHQHKKLVNRDLKTQSGSEMKKFLSTLTIDGVTRSD |
| | | QGLYTCAASSGLMTKKNSTFVRVHEKPFVAFGSGME |
| | | SLVEATVGERVRIPAKYLGYPPPEIKWYKNGIPLESNH |
| | | TIKAGHVLTIMEVSERDTGNYTVILTNPISKEKQSHVV |
| | | SLVVYVPPQIGEKSLISPVDSYQYGTTQTLTCTVYAIPP |
| | | PHHIHWYWQLEEECANEPSQAVSVTNPYPCEEWRSV |
| | | EDFQGGNKIEVNKNQFALIEGKNKTVSTLVIQAANVS |
| | | ALYKCEAVNKVGRGERVISFHVTRGPEITLQPDMQPT |
| | | EQESVSLWCTADRSTFENLTWYKLGPQPLPIHVGELPT |
| | | PVCKNLDTLWKLNATMFSNSTNDILIMELKNASLQDQ |
| | | GDYVCLAQDRKTKKRHCVVRQLTVLERVAPTITGNL |
| | | ENQTTSIGESIEVSCTASGNPPPQIMWFKDNETLVEDS |
| | | GIVLKDGNRNLTIRRVRKEDEGLYTCQACSVLGCAKV |
| | | EAFFIIEGAQEKTNLEIIILVGTAVIAMFFWLLLVIILRT |
| | | VKRANGGELKTGYLSIVMDPDELPLDEHCERLPYDAS |
| | | KWEFPRDRLKLGKPLGRGAFGQVIEADAFGIDKTATC |
| | | RTVAVKMLKEGATHSEHRALMSELKILIHIGHHLNVV |
| | | NLLGACTKPGGPLMVIVEFCKFGNLSTYLRSKRNEFV |
| | | PYKTKGARFRQGKDYVGAIPVDLKRRLDSITSSQSSAS |
| | | SGFVEEKSLSDVEEEEAPEDLYKDFLTLEHLICYSFQV |
| | | AKGMEFLASRKCIHRDLAARNILLSEKNVVKICDFGL |
| | | ARDIYKDPDYVRKGDARLPLKWMAPETIFDRVYTIQS |
| | | DVWSFGVLLWEIFSLGASPYPGVKIDEEFCRRLKEGTR |
| | | MRAPDYTTPEMYQTMLDCWHGEPSQRPTFSELVEHL |
| | | GNLLQANAQQDGKDYIVLPISETLSMEEDSGLSLPTSP |
| | | VSCMEEEEVCDPKFHYDNTAGISQYLQNSKRKSRPVS |
| | | VKTFEDIPLEEPEVKVIPDDNQTDSGMVLASEELKTLE |
| | | DRTKLSPSFGGMVPSKSRESVASEGSNQTSGYQSGYH |
| | | SDDTDTTVYSSEEAELLKLIEIGVQTGSTAQILQPDSGT |
| | | TLSSPPV |
| 345 | Macaca VEGFR2 | MASKVLLAVALWLCVETRAASVGLPSVSLDLPRLSIQ |
| | domain | KDILTIKANTTLQITCRGQRDLDWLWPNNQSGSEQRV |
| | | EVTECSDGLFCKTLTIPKVIGNDTGAYKCFYRETDLAS |
| | | VIYVYVQDYRSPFIASVSDQHGVVYITENKNKTVVIPC |
| | | LGSISNLNVSLCARYPEKRFVPDGNRISWDSKKGFTIPS |
| | | YMISYAGMVFCEAKINDESYQSIMYIVVVVGYRIYDV |
| | | VLSPSHGVELSVGEKLVLNCTARTELNVGIDENWEYP |
| | | SSKHQHKKLVNRDLKTQSGSEMKKFLSTLTIDGVTRS |
| | | DQGLYTCAASSGLMTKKNSTFVRVHEKPFVAFGSGM |
| | | ESLVEATVGERVRIPVKYLGYPPPEIKWYKNGIPLESN |
| | | HTVKVGHVLTIMEVSERDTGNYTVILTNPISKEKQSHV |
| | | VSLVVYVPPQIGEKSLISPVDSYQYGTTQTLTCTVYAIP |
| | | PPHHIHWYWQLEEECPNEPSQAVSVTNPYPCEEWRSV |
| | | EDFQGGNKIEVNKNQFALIEGKNKTVSTLVIQAANVS |
| | | ALYKCEAVNKVGRGERVISFHVTRGPEITLQPDLQPTE |
| | | QESVSLWCTADKSTFENLTWYKLGPQPLPVHVGELPT |
| | | PVCKNLDTLWKLNATIFSNSTNDILIMELKNASLQDQ |
| | | GDYVCVAQDRKTKKRHCVVRQLTVLERVAPMITGNL |
| | | ENQTTSIGETIEVSCTASGNPPPQIMWFKDNETLVEDS |
| | | GIVLKDGNRNLTIRRVRKEDEGLYTCQACSVLGCAKV |
| | | EAFFIIEGAQEKTNLEIIILVGTAVIAMFFWLLLVIILRT |
| | | VKRANGGELKTGYLSIVMDPDELPLDEHCERLPYDAS |
| | | KWEFPRDRLKLGKPLGRGAFGQVIEADAFGIDKTATC |
| | | RTVAVKMLKEGATHSEHRALMSELKILIHIGHHLNVV |
| | | NLLGACTKPGGPLMVIVEFCKFGNLSTYLRSKRNEFV |
| | | PYKTKGARFRQGKDYVGAIPVDLKRRLDSITSSQSSAS |
| | | SGFVEEKSLSDVEEEEAPEDLYKDFLTLEHLICYSFQV |
| | | AKGMEFLASRKCIHRDLAARNILLSEKNVVKICDFGL |
| | | ARDIYKDPDYVRKGDARLPLKWMAPETIFDRVYTIQS |
| | | DVWSFGVLLWEIFSLGASPYPGVKIDEEFCRRLKEGTR |
| | | MRAPDYTTPEMYQTMLDCWHGEPSQRPTFSELVEHL |
| | | GNLLQANAQQDGKDYIVLPISETLSMEEDSGLSLPTSP |
| | | VSCMEEEEVCDPKFHYDNTAGISQYLQNSKRKSRPVS |
| | | VKTFEDIPLEEPEVKVIPDDNQTDSGMVLASEELKTLE |
| | | DRTKLAPSFSGMVSSKSRESVASEGSNQTSGYQSGYH |
| | | SDDTDTTVYSSEEAELLKLIEIGVQTGSTAQILQPDSGT |
| | | TLSSPPV |

### Bispecific Antibody

In some embodiments, the binding agent is a multispecific antibody that comprises (a) an antibody or fragment thereof that specifically binds to PD-L1 and (b) an antibody or fragment thereof that specifically binds to VEGF. In some preferred embodiments, the binding agent is a bispecific antibody that comprises (a) an antibody or fragment thereof that specifically binds to PD-L1 and (b) an antibody or fragment thereof that specifically binds to VEGF.

In some preferred embodiments, the bispecific antibody comprises an anti-VEGF antibody (e.g., anti-VEGF-A antibody) or fragment thereof, and an antagonist of the PD1/PD-L1 interaction and/or a PD-1 or PD-L1 blocking antibody. In some embodiments, the bispecific antibody comprises an anti-VEGF antibody (e.g., anti-VEGF-A antibody) or fragment thereof, and a PD-1 or PD-L1 blocking antibody. In some preferred embodiments, the bispecific antibody comprises an anti-VEGF antibody (e.g., anti-VEGF-A antibody) or fragment thereof, and an anti-PD-1 or anti-PD-L1 antibody or fragment thereof. For example, the anti-PD-1 antibody can be selected from any anti-PD-1 antibody known in the art such as pembrolizumab or nivolumab. An exemplary anti-PD-L1 antibody includes atezolizumab.

In some embodiments, the bispecific antibody comprises any combination of one or more full-length antibodies, such as a human antibody, a humanized antibody, a chimeric antibody, a monoclonal antibody, or a polyclonal antibody, and/or one or more antibody fragments, such as a Fab, a Fab′, a F(ab′)2, an Fv, an scFv, a Fab′-SH, an sdAb, or a VHH. In some embodiments, the bispecific antibody comprises a full-length antibody and an antibody fragment, such as a Fab, a Fab′, a F(ab′)2, an Fv, an scFv, a Fab′-SH, an sdAb, or a VHH.

In some embodiments, the anti-VEGF antibody or fragment thereof comprises or consists of a human antibody, a humanized antibody, or a chimeric antibody, preferably a humanized antibody. In some embodiments, the anti-VEGF antibody or fragment thereof is a monoclonal antibody, for example a monoclonal human antibody, a monoclonal humanized antibody, or a monoclonal chimeric antibody. In some embodiments, the anti-VEGF antibody or fragment thereof is a monoclonal humanized antibody.

In some embodiments, the anti-PD-L1 antibody or fragment thereof comprises or consists of an antibody fragment selected from an Fab, an Fab′, an F(ab′)2, an Fv, an scFv, an Fab′-SH, an sdAb, and a VHH, preferably a sdAb, most preferably a VHH. In some embodiments, the anti-PD-L1 antibody or fragment thereof is an anti-PD-L1 single domain antibody. In some embodiments, the anti-PD-L1 antibody or fragment thereof is an anti-PD-L1 VHH.

In some embodiments, the bispecific antibody comprises an anti-VEGF antibody (e.g., anti-VEGF-A antibody) and/or an anti-PD-L1 single domain antibody.

In some embodiments, the bispecific antibody has monovalent binding to VEGF and/or to PD-L1. In some embodiment the bispecific antibody has bivalent binding to VEGF and/or to PD-L1. In some preferred embodiments, the bispecific antibody has bivalent binding to VEGF and to PD-L1.

In some embodiments, the anti-VEGF antibody (e.g., anti-VEGF-A antibody) or fragment thereof is a monovalent or bivalent antibody or fragment thereof. In some embodiments, the anti-PD-L1 antibody or fragment thereof is a monovalent or bivalent antibody or fragment thereof. In some preferred embodiments, the anti-VEGF antibody (e.g., anti-VEGF-A antibody) or fragment thereof is a bivalent anti-VEGF antibody or fragment thereof and/or the anti-PD-L1 antibody or fragment thereof is a bivalent antibody or fragment thereof. In some preferred embodiments, the anti-VEGF antibody (e.g., anti-VEGF-A antibody) is a bivalent anti-VEGF antibody and the anti-PD-L1 is a monovalent single domain antibody.

Example anti-PD-L1 Sequences

In some embodiments, the bispecific antibody comprises an anti-PD-L1 antibody or fragment thereof. In preferred embodiments, the bispecific antibody comprises an anti-PD-L1 single domain antibody.

Example anti-PD-L1 HCDRs and VHH sequences are provided below.

| | |
|---|---|
| Kabat HCDR1 | SYWMY (SEQ ID NO: 81) |
| Kabat HCDR2 | SINSDSSSTYYRDSVKG (SEQ ID NO: 82) |
| Kabat HCDR3 | DPGGYA (SEQ ID NO: 83) |
| IMGT HCDR1 | GFTFSSYW (SEQ ID NO: 84) |
| IMGT HCDR2 | INSDSSST (SEQ ID NO: 85) |
| IMGT HCDR3 | AKDPGGYA (SEQ ID NO: 86) |
| VHH | EVQLQESGGGLVQPGGSLRLSCAASGFTFSS |
| | YWMYWLRQAPGKGLEWVSSINSDSSSTYYRD |
| | SVKGRFTISRDNAKNTLYLQMNSLKSEDTAV |
| | YYCAKDPGGYAKGQGTQVTVSS |
| | (SEQ ID NO: 87) |

In some embodiments, the anti-PD-L1 antibody or fragment thereof comprises a heavy chain variable region (e.g. an antigen-binding fragment from a camelid-derived heavy chain antibody (VHH)), wherein the heavy chain variable region (e.g. the antigen-binding fragment from a camelid-derived heavy chain antibody (VHH)) comprises: (i) a HCDR1 comprising or consisting of the amino acid sequence shown in SEQ ID NO: 81, or a variant thereof having up to three amino acid substitutions, additions or deletions; (ii) a HCDR2 comprising or consisting of the amino acid sequence shown in SEQ ID NO: 82, or a variant thereof having up to three amino acid substitutions, additions or deletions; and (iii) a HCDR3 comprising or consisting of the amino acid sequence shown in SEQ ID NO: 83, or a variant thereof having up to three amino acid substitutions, additions or deletions, wherein the CDRs are defined according to the Kabat numbering system.

In some embodiments, the anti-PD-L1 antibody or fragment thereof comprises a heavy chain variable region (e.g. an antigen-binding fragment from a camelid-derived heavy chain antibody (VHH)), wherein the heavy chain variable region comprises (e.g. the antigen-binding fragment from a camelid-derived heavy chain antibody (VHH)): (i) a HCDR1 comprising or consisting of the amino acid sequence shown in SEQ ID NO: 84, or a variant thereof having up to three amino acid substitutions, additions or deletions; (ii) a HCDR2 comprising or consisting of the amino acid sequence shown in SEQ ID NO: 85, or a variant thereof having up to three amino acid substitutions, additions or deletions; and (iii) a HCDR3 comprising or consisting of the amino acid sequence shown in SEQ ID NO: 86, or a variant thereof having up to three amino acid substitutions, additions or deletions, wherein the CDRs are defined according to the IMGT numbering system.

In some embodiments, the CDR variants have up to two amino acid substitutions, additions or deletions. In some embodiments, the CDR variants have up to one amino acid substitution, addition or deletion. In some embodiments, the CDR variants have up to three amino acid substitutions. In some embodiments, the CDR variants have up to two amino acid substitutions. In some embodiments, the CDR variants have up to one amino acid substitution.

In some embodiments, the anti-PD-L1 antibody or fragment thereof comprises or consists of the amino acid sequence shown in SEQ ID NO: 87, or an amino acid sequence having at least 80%, 85%, 90%, 95%, 96%, 97%, 98%, or 99% identity to SEQ ID NO: 87 and/or one or more (e.g., 1, 2, 3, 4, 5, 6, 7, 8, 9, or 10) amino acid additions, deletions and/or substitutions in a framework region thereof.

In some embodiments, the anti-PD-L1 antibody or fragment thereof comprises or consists of the amino acid sequence of SEQ ID NO: 87, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, and comprises a heavy chain variable region comprising: a HCDR1 comprising or consisting of the amino acid sequence of SEQ ID NO: 81; a HCDR2 comprising or consisting of the amino acid sequence of SEQ ID NO: 82; and a HCDR3 comprising or consisting of the amino acid sequence of SEQ ID NO: 83.

In some embodiments, the anti-PD-L1 antibody or fragment thereof comprises or consists of the amino acid sequence of SEQ ID NO: 87, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, and comprises a heavy chain variable region comprising: a HCDR1 comprising or consisting of the amino acid sequence of SEQ ID NO: 84; a HCDR2 comprising or consisting of the amino acid sequence of SEQ ID NO: 85; and a HCDR3 comprising or consisting of the amino acid sequence of SEQ ID NO: 86.

In some embodiments, the anti-PD-L1 antibody or fragment thereof comprises or consists of the amino acid sequence of SEQ ID NO: 87. In some embodiments, the ani-PD-L1 antibody or fragment thereof consists of the amino acid sequence of SEQ ID NO: 87

### Example Anti-VEGF Sequences

In some embodiments, the bispecific antibody comprises an anti-VEGF antibody or fragment thereof (e.g., anti-VEGF-A antibody or fragment thereof). In preferred embodiments, the bispecific antibody comprises an anti-VEGF antibody (e.g., anti-VEGF-A antibody).

Example anti-VEGF antibody CDRs, variable region sequences, constant region sequences, and heavy and light chain sequences are provided below:

| | |
|---|---|
| Kabat HCDR1 | NYGMN (SEQ ID NO: 88) |
| Kabat HCDR2 | WINTYTGEPTYAADFKR (SEQ ID NO: 89) |
| Kabat HCDR3 | YPHYYGSSHWYFDV (SEQ ID NO: 90) |
| Kabat LCDR1 | SASQDISNYLN (SEQ ID NO: 91) |
| Kabat LCDR2 | FTSSLHS (SEQ ID NO: 92) |
| Kabat LCDR3 | QQYSTVPWT (SEQ ID NO: 93) |
| IMGT HCDR1 | GYTFTNYG (SEQ ID NO: 94) |
| IMGT HCDR2 | INTYTGEP (SEQ ID NO: 95) |
| IMGT HCDR3 | AKYPHYYGSSHWYFDV (SEQ ID NO: 96) |
| IMGT LCDR1 | QDISNY (SEQ ID NO: 97) |
| IMGT LCDR2 | FTS (SEQ ID NO: 98) |
| IMGT LCDR3 | QQYSTVPWT (SEQ ID NO: 99) |
| VH | EVQLVESGGGLVQPGGSLRLSCAASGYTFTNYGMNWVRQAPGKGL |
| | EWVGWINTYTGEPTY AADFKRRFTFSLDTSKSTAYLQMNSLRAEDT |
| | AVYYCAKYPHYYGSSHWYFDVWGQGTLVTVSS (SEQ ID NO: 100) |
| VL | DIQMTQSPSSLSASVGDRVTITCSASQDISNYLNWYQQKPGKAPKVLI |
| | YFTSSLHSGVPSRFSGSGSGTDFTLTISSLQPEDFATYYCQQYSTVPW |
| | TFGQGTKVEIK (SEQ ID NO: 101) |
| CH1 | ASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVD |
| | KKVEPKSC (SEQ ID NO: 102) |
| Fc | DKTHTCPPCPAPEAAGGPSVFLFPPKPKDTLMISRTPEVTCVVVDVSH |
| | EDPEVKFNWYVDGVEVHNAKTKPREEQYNSTYRVVSVLTVLHQDW |
| | LNGKEYKCKVSNKALPAPIEKTISKAKGQPREPQVYTLPPSRDELTK |
| | NQVSLTCLVKGFYPSDIAVEWESNGQPENNYKTTPPVLDSDGSFFLY |
| | SKLTVDKSRWQQGNVFSCSVMHEALHNHYTQKSLSLSPG (SEQ ID |
| | NO: 103) |
| CL | RTVAAPSVFIFPPSDEQLKSGTASVVCLLNNFYPREAKVQWKVDNAL |
| | QSGNSQESVTEQDSKDSTYSLSSTLTLSKADYEKHKVYACEVTHQGL |
| | SSPVTKSFNRGEC (SEQ ID NO: 104) |
| Heavy chain | EVQLVESGGGLVQPGGSLRLSCAASGYTFTNYGMNWVRQAPGKGL |
| | EWVGWINTYTGEPTYAADFKRRFTFSLDTSKSTAYLQMNSLRAEDT |
| | AVYYCAKYPHYYGSSHWYFDVWGQGTLVTVSSASTKGPSVFPLAPS |
| | SKSTSGGTAALGCLVKDYFPEPVTVSWNSGALTSGVHTFPAVLQSSG |
| | LYSLSSVVTVPSSSLGTQTYICNVNHKPSNTKVDKKVEPKSCDKTHT |
| | CPPCPAPEAAGGPSVFLFPPKPKDTLMISRTPEVTCVVVDVSHEDPEV |
| | KFNWYVDGVEVHNAKTKPREEQYNSTYRVVSVLTVLHQDWLNGK |
| | EYKCKVSNKALPAPIEKTISKAKGQPREPQVYTLPPSRDELTKNQVSL |
| | TCLVKGFYPSDIAVEWESNGQPENNYKTTPPVLDSDGSFFLYSKLTV |
| | DKSRWQQGNVFSCSVMHEALHNHYTQKSLSLSPG (SEQ ID NO: 105) |
| Light chain | DIQMTQSPSSLSASVGDRVTITCSASQDISNYLNWYQQKPGKAPKVLI |
| | YFTSSLHSGVPSRFSGSGSGTDFTLTISSLQPEDFATYYCQQYSTVPW |
| | TFGQGTKVEIKRTVAAPSVFIFPPSDEQLKSGTASVVCLLNNFYPREA |
| | KVQWKVDNALQSGNSQESVTEQDSKDSTYSLSSTLTLSKADYEKHK |
| | VYACEVTHQGLSSPVTKSFNRGEC (SEQ ID NO: 106) |

In some embodiments, the anti-VEGF antibody or fragment thereof comprises a constant region, preferably derived from a human antibody, preferably the constant region is selected from the constant region of human IgG1, IgG2, IgG3 or IgG4.

In some embodiments, the anti-VEGF antibody or fragment thereof comprises an Fc region comprising one or more mutations which reduce or abolish immune effect functions. Suitable mutations are known in the art and may include one or more amino acid substitutions (see e.g. Wilkinson, I., et al., 2021. FcPLoS One, 16(12), p. e0260954; and Liu, R., et al., 2020. Antibodies, 9(4), p.64). In some embodiments, the anti-VEGF antibody or fragment thereof comprises an IgG1 Fc region comprising a L234A and/or a L235A mutation. In some embodiments, the anti-VEGF antibody or fragment thereof comprises an IgG1 Fc region comprising L234A and L235A mutations. Amino acids are numbered according to the Eu numbering (Eu-index) as set forth in Kabat (Kabat, E. A.; National Institutes of Health (U.S.) Office of the Director. Sequences of Proteins of Immunological Interest, 5th ed.; DIANE Publishing: Collingdale, PA, USA, 1991). For example, these may correspond to L240A and L241A in SEQ ID NO: 105.

In some embodiments, the anti-VEGF antibody or fragment thereof comprises a CH1 domain, preferably comprising or consisting of the amino acid sequence shown in SEQ ID NO: 102 or an amino acid sequence having at least 80%, 85%, 90%, 95%, 96%, 97%, 98%, or 99% identity to the amino acid sequence shown in SEQ ID NO: 102.

In some embodiments, the anti-VEGF antibody or fragment thereof comprises a IgG1 Fc region, preferably comprising or consisting of the amino acid sequence shown in SEQ ID NO: 103 or an amino acid sequence having at least 80%, 85%, 90%, 95%, 96%, 97%, 98%, or 99% identity to the amino acid sequence shown in SEQ ID NO: 103.

In some embodiments, the anti-VEGF antibody or fragment thereof comprises a CL domain, preferably comprising or consisting of the amino acid sequence shown in SEQ ID NO: 104 or an amino acid sequence having at least 80%, 85%, 90%, 95%, 96%, 97%, 98%, or 99% identity to the amino acid sequence shown in SEQ ID NO: 104.

In some embodiments, the anti-VEGF antibody or fragment thereof comprises a heavy chain variable region and/or a light chain variable region. In some embodiments, the anti-VEGF antibody or fragment thereof comprises a heavy chain variable region and a light chain variable region.

In some embodiments, a heavy chain variable region of the anti-VEGF antibody or fragment thereof comprises: (i) a HCDR1 comprising or consisting of the amino acid sequence shown in SEQ ID NO: 88, or a variant thereof having up to three amino acid substitutions, additions or deletions; (ii) a HCDR2 comprising or consisting of the amino acid sequence shown in SEQ ID NO: 89, or a variant thereof having up to three amino acid substitutions, additions or deletions; and (iii) a HCDR3 comprising or consisting of the amino acid sequence shown in SEQ ID NO: 90, or a variant thereof having up to three amino acid substitutions, additions or deletions; wherein the CDRs are defined according to the Kabat numbering system.

In some embodiments, a heavy chain variable region of the anti-VEGF antibody or fragment thereof comprises: (i) a HCDR1 comprising or consisting of the amino acid sequence shown in SEQ ID NO: 94, or a variant thereof having up to three amino acid substitutions, additions or deletions; (ii) a HCDR2 comprising or consisting of the amino acid sequence is shown in SEQ ID NO: 95, or a variant thereof having up to three amino acid substitutions, additions or deletions; and (iii) a HCDR3 comprising or consisting of the amino acid sequence is shown in SEQ ID NO: 96, or a variant thereof having up to three amino acid substitutions, additions or deletions, wherein the CDRs are defined according to the IMGT numbering system.

In some embodiments, a light chain variable region of the anti-VEGF antibody or fragment thereof comprises: (i) a LCDR1 comprising or consisting of the amino acid sequence shown in SEQ ID NO: 91, or a variant thereof having up to three amino acid substitutions, additions or deletions; (ii) a LCDR2 comprising or consisting of the amino acid sequence shown in SEQ ID NO: 92, or a variant thereof having up to three amino acid substitutions, additions or deletions; and (iii) a LCDR3 comprising or consisting of the amino acid sequence shown in SEQ ID NO: 93, or a variant thereof having up to three amino acid substitutions, additions or deletions, wherein the CDRs are defined according to the Kabat numbering system.

In some embodiments, a light chain variable region of the anti-VEGF antibody or fragment thereof comprises: (i) a LCDR1 comprising or consisting of the amino acid sequence of SEQ ID NO: 97, or a variant thereof having up to three amino acid substitutions, additions or deletions; (ii) a LCDR2 comprising or consisting of the amino acid sequence of SEQ ID NO: 98, or a variant thereof having up to three amino acid substitutions, additions or deletions; and (iii) a LCDR3 comprising or consisting of the amino acid sequence shown in SEQ ID NO: 99, or a variant thereof having up to three amino acid substitutions, additions or deletions, wherein the CDRs are defined according to the IMGT numbering system.

In some embodiments, a heavy chain variable region of the anti-VEGF antibody or fragment thereof comprises: (i) a HCDR1 comprising or consisting of the amino acid sequence shown in SEQ ID NO: 88, or a variant thereof having up to three amino acid substitutions, additions or deletions; (ii) a HCDR2 comprising or consisting of the amino acid sequence shown in SEQ ID NO: 89, or a variant thereof having up to three amino acid substitutions, additions or deletions; and (iii) a HCDR3 comprising or consisting of the amino acid sequence shown in SEQ ID NO: 90, or a variant thereof having up to three amino acid substitutions, additions or deletions; and a light chain variable region of the anti-VEGF antibody or fragment thereof comprises: (i) a LCDR1 comprising or consisting of the amino acid sequence shown in SEQ ID NO: 91, or a variant thereof having up to three amino acid substitutions, additions or deletions; (ii) a LCDR2 comprising or consisting of the amino acid sequence shown in SEQ ID NO: 92, or a variant thereof having up to three amino acid substitutions, additions or deletions; and (iii) a LCDR3 comprising or consisting of the amino acid sequence shown in SEQ ID NO: 93, or a variant thereof having up to three amino acid substitutions, additions or deletions, wherein the CDRs are defined according to the Kabat numbering system.

In some embodiments, a heavy chain variable region of the anti-VEGF antibody or fragment thereof comprises: (i) a HCDR1 comprising or consisting of the amino acid sequence shown in SEQ ID NO: 94, or a variant thereof having up to three amino acid substitutions, additions or deletions; (ii) a HCDR2 comprising or consisting of the amino acid sequence shown in SEQ ID NO: 95, or a variant thereof having up to three amino acid substitutions, additions or deletions; and (iii) a HCDR3 comprising or consisting of the amino acid sequence shown in SEQ ID NO: 96, or a variant thereof having up to three amino acid substitutions, additions or deletions; and a light chain variable region of the anti-VEGF antibody or fragment thereof comprises: (i) a LCDR1 comprising or consisting of the amino acid sequence shown in SEQ ID NO: 97, or a variant thereof having up to three amino acid substitutions, additions or deletions; (ii) a LCDR2 comprising or consisting of the amino acid sequence shown in SEQ ID NO: 98, or a variant thereof having up to three amino acid substitutions, additions or deletions; and (iii) a LCDR3 comprising or consisting of the amino acid sequence shown in SEQ ID NO: 99, or a variant thereof having up to three amino acid substitutions, additions or deletions, wherein the CDRs are defined according to the IMGT numbering system.

In some embodiments, the CDR variants have up to two amino acid substitutions, additions or deletions. In some embodiments, the CDR variants have up to one amino acid substitution, addition or deletion. In some embodiments, the CDR variants have up to three amino acid substitutions. In some embodiments, the CDR variants have up to two amino acid substitutions. In some embodiments, the CDR variants have up to one amino acid substitution.

In some embodiments, a heavy chain variable region of the anti-VEGF antibody or fragment thereof comprises or consists of the amino acid sequence shown in SEQ ID NO: 100, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof. In some embodiments, a heavy chain variable region of the anti-VEGF antibody or fragment thereof comprises or consists of the amino acid sequence of SEQ ID NO: 100.

In some embodiments, a light chain variable region of the anti-VEGF antibody or fragment thereof comprises or consists of the amino acid sequence shown in SEQ ID NO: 101 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof. In some embodiments, a light chain variable region of the anti-VEGF antibody or fragment thereof comprises or consists of the amino acid sequence of SEQ ID NO: 101.

In some embodiments, a heavy chain variable region of the anti-VEGF antibody or fragment thereof comprises or consists of the amino acid sequence shown in SEQ ID NO: 100, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, and a light chain variable region of the anti-VEGF antibody or fragment thereof comprises or consists of the amino acid sequence shown in SEQ ID NO: 101 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof.

In some embodiments, a heavy chain variable region of the anti-VEGF antibody or fragment thereof comprises or consists of the amino acid sequence of SEQ ID NO: 100, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the heavy chain variable region comprises: a HCDR1 comprising or consisting of the amino acid sequence of SEQ ID NO: 88; a HCDR2 comprising or consisting of the amino acid sequence of SEQ ID NO: 89; and a HCDR3 comprising or consisting of the amino acid sequence of SEQ ID NO: 90; and a light chain variable region of the anti-VEGF antibody or fragment thereof comprises or consists of the amino acid sequence of SEQ ID NO: 101 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the light chain variable region comprises: a LCDR1 comprising or consisting of the amino acid sequence of SEQ ID NO: 91; a LCDR2 comprising or consisting of the amino acid sequence of SEQ ID NO: 92; and a LCDR3 comprising or consisting of the amino acid sequence of SEQ ID NO: 93.

In some embodiments, a heavy chain variable region of the anti-VEGF antibody or fragment thereof comprises or consists of the amino acid sequence of SEQ ID NO: 100, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the heavy chain variable region comprises: a HCDR1 comprising or consisting of the amino acid sequence of SEQ ID NO: 94; a HCDR2 comprising or consisting of the amino acid sequence of SEQ ID NO: 95; and a HCDR3 comprising or consisting of the amino acid sequence of SEQ ID NO: 96; and a light chain variable region of the anti-VEGF antibody or fragment thereof comprises or consists of the amino acid sequence of SEQ ID NO: 101 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the light chain variable region comprises: a LCDR1 comprising or consisting of the amino acid sequence of SEQ ID NO: 97; a LCDR2 comprising or consisting of the amino acid sequence of SEQ ID NO: 98; and a LCDR3 comprising or consisting of the amino acid sequence of SEQ ID NO: 99.

In some embodiments, a heavy chain variable region of the anti-VEGF antibody or fragment thereof comprises or consists of the amino acid sequence shown in SEQ ID NO: 100, and a light chain variable region of the anti-VEGF antibody or fragment thereof comprises or consists of the amino acid sequence shown in SEQ ID NO: 101. In some embodiments, a heavy chain variable region of the anti-VEGF antibody or fragment thereof consists of the amino acid sequence of SEQ ID NO: 100, and a light chain variable region of the anti-VEGF antibody or fragment thereof consists of the amino acid sequence of SEQ ID NO: 101.

In some embodiments, a heavy chain of the anti-VEGF antibody or fragment thereof comprises or consists of the amino acid sequence of SEQ ID NO: 105, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof. In some embodiments, a heavy chain of the anti-VEGF antibody or fragment thereof comprises or consists of the amino acid sequence of SEQ ID NO: 105.

In some embodiments, a light chain of the anti-VEGF antibody or fragment thereof comprises or consists of the amino acid sequence of SEQ ID NO: 106 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof. In some embodiments, a light chain of the anti-VEGF antibody or fragment thereof comprises or consists of the amino acid sequence of the amino acid sequence of SEQ ID NO: 106.

In some embodiments, a heavy chain of the anti-VEGF antibody or fragment thereof comprises or consists of the amino acid sequence of the amino acid sequence of SEQ ID NO: 105, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof; and a light chain of the anti-VEGF antibody or fragment thereof comprises or consists of the amino acid sequence of SEQ ID NO: 106 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof.

In some embodiments, a heavy chain of the anti-VEGF antibody or fragment thereof comprises or consists of the amino acid sequence of SEQ ID NO: 105, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the heavy chain variable region comprises: a HCDR1 comprising or consisting of the amino acid sequence of SEQ ID NO: 88; a HCDR2 comprising or consisting of the amino acid sequence of SEQ ID NO: 89; and a HCDR3 comprising or consisting of the amino acid sequence of SEQ ID NO: 90; and a light chain of the anti-VEGF antibody or fragment thereof comprises or consists of the amino acid sequence of SEQ ID NO: 106 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the light chain variable region comprises: a LCDR1 comprising or consisting of the amino acid sequence of SEQ ID NO: 91; a LCDR2 comprising or consisting of the amino acid sequence of SEQ ID NO: 92; and a LCDR3 comprising or consisting of the amino acid sequence of SEQ ID NO: 93.

In some embodiments, a heavy chain of the anti-VEGF antibody or fragment thereof comprises or consists of the amino acid sequence of SEQ ID NO: 105, or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the heavy chain variable region comprises: a HCDR1 comprising or consisting of the amino acid sequence of SEQ ID NO: 94; a HCDR2 comprising or consisting of the amino acid sequence of SEQ ID NO: 95; and a HCDR3 comprising or consisting of the amino acid sequence of SEQ ID NO: 96; and a light chain of the anti-VEGF antibody or fragment thereof comprises or consists of the amino acid sequence of SEQ ID NO: 106 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, wherein the light chain variable region comprises: a LCDR1 comprising or consisting of the amino acid sequence of SEQ ID NO: 97; a LCDR2 comprising or consisting of the amino acid sequence of SEQ ID NO: 98; and a LCDR3 comprising or consisting of the amino acid sequence of SEQ ID NO: 99.

In some embodiments, a heavy chain of the anti-VEGF antibody or fragment thereof comprises or consists of the amino acid sequence of SEQ ID NO: 105; and a light chain of the anti-VEGF antibody or fragment thereof comprises or consists of the amino acid sequence of SEQ ID NO: 106. In some embodiments, a heavy chain of the anti-VEGF antibody or fragment thereof consists of the amino acid sequence of SEQ ID NO: 105; and a light chain of the anti-VEGF antibody or fragment thereof consists of the amino acid sequence of SEQ ID NO: 106.

In some embodiments, the anti-VEGF antibody or fragment thereof comprises or consists of two heavy chains comprising or consisting of the amino acid sequence of SEQ ID NO: 105; and two light chain comprising or consisting of the amino acid sequence of SEQ ID NO: 106.

In some embodiments, the anti-VEGF antibody or fragment thereof comprises or consists of two heavy chains consisting of the amino acid sequence of SEQ ID NO: 105; and two light chains consisting of the amino acid sequence of SEQ ID NO: 106.

In some embodiments, the anti-VEGF antibody or fragment thereof is Bevacizumab or a fragment thereof. In some embodiments, the anti-VEGF antibody or fragment thereof is Bevacizumab.

### Example Bispecific Antibody Sequences

In some embodiments, the bispecific antibody comprises an anti-VEGF antibody (e.g., anti-VEGF-A antibody) and an anti-PD-L1 single domain antibody.

In some embodiments, the bispecific antibody comprises one or more anti-PD-L1 single domain antibodies. In some embodiments, the bispecific antibody comprises at least two anti-PD-L1 single domain antibodies. In some preferred embodiments, the bispecific antibody comprises two anti-PD-L1 single domain antibodies.

The one or more anti-PD-L1 single domain antibodies may be fused to the anti-VEGF antibody by any suitable method. In some embodiments, each anti-PD-L1 single domain antibody is terminally-fused to the anti-VEGF antibody. In some embodiments, each anti-PD-L1 single domain antibody is fused to: (i) the C-terminus of a heavy chain; (ii) the N-terminus of a heavy chain; (iii) the C-terminus of a light chain; or (iv) the N-terminus of a light chain.

In preferred embodiments, each anti-PD-L1 single domain antibodies is fused to the C-terminus of an anti-VEGF antibody, preferably the C-terminus of a heavy chain. The bispecific antibody preferably comprises an anti-VEGF antibody and two anti-PD-L1 VHHs, wherein each heavy chain of the anti-VEGF antibody is fused to an anti-PD-L1 VHH, preferably at the C-terminus of the heavy chain of the anti-VEGF antibody.

The VHH can be fused to the anti-VEGF antibody by a linker, e.g. a peptide linker, for example a glycine and serine-rich linker (GS-linker) or any suitable linker known in the art.

An example linker is provided by SEQ ID NO: 107, shown below:

(SEQ ID NO: 107)
GGGGSGGGGSG
Example bispecific antibody linker

In some embodiments, the bispecific antibody comprises a linker, preferably comprising or consisting of the amino acid sequence shown in SEQ ID NO: 107 or an amino acid sequence having at least 80%, 85%, 90%, 95%, 96%, 97%, 98%, or 99% identity to the amino acid sequence shown in SEQ ID NO: 107.

In some embodiments, the bispecific antibody comprises an anti-VEGF antibody comprising two heavy chains; and two anti-PD-L1 VHHs, wherein each heavy chain of the anti-VEGF antibody is fused to an anti-PD-L1 VHH through a linker comprising the amino acid sequence shown in SEQ ID NO: 107 or an amino acid sequence having at least 80%, 85%, 90%, 95%, 96%, 97%, 98%, or 99% identity thereto.

In some embodiments, the anti-VEGF antibody or fragment thereof specifically binds to VEGF-A. For example, the bispecific antibody can comprise two VHHs, wherein each VHH is fused to the C-terminus of an anti-VEGF-A antibody, preferably the C-terminus of each heavy chain of the anti-VEGF-A antibody. The bispecific antibody can therefore comprise an anti-VEGF-A antibody and two anti-PD-L1 VHHs, wherein each heavy chain of the anti-VEGF-A antibody is fused to an anti-PD-L1 VHH through a linker, such as the linker comprising the amino acid sequence shown in SEQ ID NO: 107 or an amino acid sequence having at least 80%, 85%, 90%, 95%, 96%, 97%, 98%, or 99% identity thereto.

The bispecific antibody disclosed herein preferably comprises an anti-VEGF antibody or fragment thereof comprising the HCDRs 1-3 and the LCDRs 1-3, or variants thereof having up to three amino acid substitutions, additions or deletions; and an anti-PD-L1 single domain comprising the HCDRs 1-3 of the below table, or variants thereof having up to three amino acid substitutions, additions or deletions.

| | Kabat numbering system | | IMGT numbering system | |
|---|---|---|---|---|
| Anti-PD-L1 | SYWMY | SEQ ID | GFTFSSYW | SEQ ID |
| HCDR1 | | NO: 81 | | NO: 84 |
| Anti-PD-L1 | SINSDSSSTYYRDSVKG | SEQ ID | INSDSSST | SEQ ID |
| HCDR2 | | NO: 82 | | NO: 85 |
| Anti-PD-L1 | DPGGYA | SEQ ID | AKDPGGYA | SEQ ID |
| HCDR3 | | NO: 83 | | NO: 86 |
| Anti-VEGF | NYGMN | SEQ ID | GYTFTNYG | SEQ ID |
| HCDR1 | | NO: 88 | | NO: 94 |
| Anti-VEGF | WINTYTGEPTY AADFKR | SEQ ID | INTYTGEP | SEQ ID |
| HCDR2 | | NO: 89 | | NO: 95 |
| Anti-VEGF | YPHYYGSSHWYFDV | SEQ ID | AKYPHYYGSSHWYFDV | SEQ ID |
| HCDR3 | | NO: 90 | | NO: 96 |
| Anti-VEGF | SASQDISNYLN | SEQ ID | QDISNY | SEQ ID |
| LCDR1 | | NO: 91 | | NO: 97 |
| Anti-VEGF | FTSSLHS | SEQ ID | FTS | SEQ ID |
| LCDR2 | | NO: 92 | | NO: 98 |
| Anti-VEGF | QQYSTVPWT | SEQ ID | QQYSTVPWT | SEQ ID |
| LCDR3 | | NO: 93 | | NO: 99 |

In some embodiments, the CDR variants have up to two amino acid substitutions, additions or deletions. In some embodiments, the CDR variants have up to one amino acid substitution, addition or deletion. In some embodiments, the CDR variants have up to three amino acid substitutions. In some embodiments, the CDR variants have up to two amino acid substitutions. In some embodiments, the CDR variants have up to one amino acid substitution.

Example bispecific antibody heavy and light chain sequences are shown below:

(SEQ ID NO: 108)
EVQLVESGGGLVQPGGSLRLSCAASGYTFTNYGMNWVRQAPGKGLEWVGW
INTYTGEPTYAADFKRRFTFSLDTSKSTAYLQMNSLRAEDTAVYYCAKY
PHYYGSSHWYFDVWGQGTLVTVSSASTKGPSVFPLAPSSKSTSGGTAALG
CLVKDYFPEPVTVSWNSGALTSGVHTFPAVLQSSGLYSLSSVVTVPSSSL
GTQTYICNVNHKPSNTKVDKKVEPKSCDKTHTCPPCPAPEAAGGPSVFLF
PPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQP
REPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLSL
SPGGGGGSGGGGSGEVQLQESGGGLVQPGGSLRLSCAASGFTFSSYWMYW
LRQAPGKGLEWVSSINSDSSSTYYRDSVKGRFTISRDNAKNTLYLQMNSL
KSEDTAVYYCAKDPGGYAKGQGTQVTVSS
Example bispecific antibody heavy chain-
VHH fusion
(SEQ ID NO: 109)
DIQMTQSPSSLSASVGDRVTITCSASQDISNYLNWYQQKPGKAPKVLIYF
TSSLHSGVPSRFSGSGSGTDFTLTISSLQPEDFATYYCQQYSTVPWTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVY+10ACEVT
HQGLSSPVTKSFNRGEC
Example bispecific antibody light chain

In some embodiments, a heavy chain of the bispecific antibody comprises or consists of the amino acid sequence shown in SEQ ID NO: 108 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof.

In some embodiments, a light chain of the bispecific antibody comprises or consists of the amino acid sequence shown in SEQ ID NO: 109 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof.

In some embodiments, a heavy chain of the bispecific antibody comprises or consists of the amino acid sequence shown in SEQ ID NO: 108 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof, and a light chain of the bispecific antibody comprises or consists of the amino acid sequence shown in SEQ ID NO: 109 or an amino acid sequence having at least 80%, 85%, 90%, 95%, 96%, 97%, 98%, or 99% identity thereto and/or one or more (e.g., 1, 2, 3, 4, 5, 6, 7, 8, 9, or 10) amino acid additions, deletions and/or substitutions in a framework region thereof.

In some embodiments, a heavy chain of the bispecific antibody comprises or consists of the amino acid sequence shown in SEQ ID NO: 108 and a light chain of the bispecific antibody comprises or consists of the amino acid sequence shown in SEQ ID NO: 109. In some embodiments, the heavy chain of the bispecific antibody consists of the amino acid sequence shown in SEQ ID NO: 108 and the light chain of the bispecific antibody consists of the amino acid sequence shown in SEQ ID NO: 109.

In some embodiments, the bispecific antibody comprises or consists of two heavy chains comprising or consisting of the amino acid sequence of SEQ ID NO: 108; and two light chains comprising or consisting of the amino acid sequence of SEQ ID NO: 109. In some embodiments, the bispecific antibody comprises or consists of two heavy chains consisting of the amino acid sequence of SEQ ID NO: 108; and two light chains consisting of the amino acid sequence of SEQ ID NO: 109.

A bispecific antibody with two heavy chain-VHH fusions with sequence SEQ ID NO: 108 and two light chains with sequence SEQ ID NO: 109 is referred to herein as “Compound 5”. It is disclosed as compound Ava-2GS-NSD in WO 2022/042719.

### Method of Treatment Options

In some embodiments, the binding agent (e.g. the bispecific antibody) and the antibody-drug conjugate are separately administered.

In some embodiments, the binding agent (e.g. the bispecific antibody) is administered in the form of one or several nucleic acids encoding the binding agent (e.g. the bispecific antibody). For example, the bispecific antibody can be administered in the form of a first nucleic acid encoding a heavy chain or a heavy chain—VHH fusion and a second nucleic acid encoding a light chain.

In some embodiments, the one or more nucleic acids can encode a bispecific antibody comprising: (i) a complementarity-determining region 1 (HCDR1) comprising or consisting of the amino acid sequence shown in SEQ ID NO: 88, (ii) a complementarity-determining region 2 (HCDR2) comprising or consisting of the amino acid sequence shown in SEQ ID NO: 89, and (iii) a complementarity-determining region 3 (HCDR3) comprising or consisting of the amino acid sequence shown in SEQ ID NO: 90; and/or (i) a complementarity-determining region 1 (LCDR1) comprising or consisting of the amino acid sequence shown in SEQ ID NO: 91, (ii) a complementarity-determining region 2 (LCDR2) comprising or consisting of the amino acid sequence shown in SEQ ID NO: 92, and (iii) a complementarity-determining region 3 (LCDR3) comprising or consisting of the amino acid sequence shown in SEQ ID NO: 93.

In some embodiments, the one or more nucleic acids can encode a bispecific antibody comprising: (i) a complementarity-determining region 1 (HCDR1) comprising or consisting of the amino acid sequence shown in SEQ ID NO: 94, (ii) a complementarity-determining region 2 (HCDR2) comprising or consisting of the amino acid sequence shown in SEQ ID NO: 95, and (iii) a complementarity-determining region 3 (HCDR3) comprising or consisting of the amino acid sequence shown in SEQ ID NO: 96; and/or (i) a complementarity-determining region 1 (LCDR1) comprising or consisting of the amino acid sequence shown in SEQ ID NO: 97, (ii) a complementarity-determining region 2 (LCDR2) comprising or consisting of the amino acid sequence shown in SEQ ID NO: 98, and (iii) a complementarity-determining region 3 (LCDR3) comprising or consisting of the amino acid sequence shown in SEQ ID NO: 99.

In some embodiments, the one or more nucleic acids can encode a bispecific antibody comprising: (i) a complementarity-determining region 1 (HCDR1) comprising or consisting of the amino acid sequence shown in SEQ ID NO: 81, (ii) a complementarity-determining region 2 (HCDR2) comprising or consisting of the amino acid sequence shown in SEQ ID NO: 82, and (iii) a complementarity-determining region 3 (HCDR3) comprising or consisting of the amino acid sequence shown in SEQ ID NO: 83.

In some embodiments, the one or more nucleic acids can encode a bispecific antibody comprising: (i) a complementarity-determining region 1 (HCDR1) comprising or consisting of the amino acid sequence shown in SEQ ID NO: 84, (ii) a complementarity-determining region 2 (HCDR2) comprising or consisting of the amino acid sequence shown in SEQ ID NO: 85, and (iii) a complementarity-determining region 3 (HCDR3) comprising or consisting of the amino acid sequence shown in SEQ ID NO: 86.

Preferably the first nucleic acid encodes an amino acid sequence shown in SEQ ID NO: 108 or an amino acid sequence having at least 80%, 85%, 90%, 95%, 96%, 97%, 98%, or 99% identity to SEQ ID NO: 108. Preferably the second nucleic acid encodes an amino acid sequence shown in SEQ ID NO: 109 or an amino acid sequence having at least 80%, 85%, 90%, 95%, 96%, 97%, 98%, or 99% identity to SEQ ID NO: 109.

In some embodiments, a dose of the binding agent (e.g. the bispecific antibody) and a dose of the antibody-drug conjugate are administered concurrently or consecutively. In some embodiments, a dose of the binding agent (e.g. the bispecific antibody) and a dose of the antibody-drug conjugate are administered concurrently. In some embodiments, a dose of the binding agent (e.g. the bispecific antibody) and a dose of the antibody-drug conjugate are administered consecutively.

In some embodiments, the binding agent (e.g. the bispecific antibody) dosage administered ranges from about 0.1 mg/kg to about 1000 mg/kg body weight, preferably from about 0.1 mg/kg to about 100 mg/kg body weight, more preferably about 1 mg/kg to about 60 mg/kg body weight. In some embodiments, the antibody-drug conjugate dosage administered ranges from about 0.5 mg/kg to about 40 mg/kg body weight, preferably about 1 mg/kg to about 30 mg/kg body weight. In some embodiments, the binding agent (e.g. the bispecific antibody) dosage administered ranges from about 0.1 mg/kg to about 100 mg/kg body weight; and the antibody-drug conjugate dosage administered ranges from about 0.5 mg/kg to about 40 mg/kg body weight. In some embodiments, the binding agent (e.g. the bispecific antibody) dosage administered ranges from about 1 mg/kg to about 60 mg/kg body weight; and the antibody-drug conjugate dosage administered ranges from about 1 mg/kg to about 30 mg/kg body weight.

The binding agent (e.g. the bispecific antibody) and/or the antibody-drug conjugate may be administered systemically, preferably the binding agent (e.g. the bispecific antibody) and the antibody-drug conjugate are administered systemically. In some embodiments, the binding agent (e.g. the bispecific antibody) and/or the antibody-drug conjugate are administered intravenously, preferably wherein the binding agent (e.g. the bispecific antibody) and the antibody-drug conjugate are administered intravenously. In some embodiments, the binding agent (e.g. the bispecific antibody) and/or the antibody-drug conjugate is administered via an IV injection or IV infusion.

In some embodiments, the subject has not been previously treated for cancer. For example, with a treatment selected from the group comprising or consisting of chemotherapy, hormone therapy, immunotherapy, radiation therapy, surgery, or a targeted therapy or any combination thereof.

In some embodiments, the subject has been previously treated for cancer. For example, with a treatment selected from the group comprising or consisting of chemotherapy, hormone therapy, immunotherapy, radiation therapy, surgery, or a targeted therapy, or any combination thereof. For example, in some embodiments the subject has previously received a chemotherapy, hormone therapy, immunotherapy, radiation therapy, surgery to remove or destroy tumours, or a targeted therapy. In some embodiments, the subject has not previously received a chemotherapy, hormone therapy, immunotherapy, radiation therapy, surgery to remove or destroy tumours, or a targeted therapy, or any combination thereof. In some embodiments, the subject has not previously received a chemotherapy, hormone therapy, immunotherapy, radiation therapy, and a targeted therapy.

In some embodiments, the subject has not been previously treated with a PD-1/PD-L1 inhibitor, such as pembrolizumab or atezolizumab.

In some embodiments, the subject has had one or more previous treatments with a PD-1/PD-L1 inhibitor, such as pembrolizumab or atezolizumab.

In some embodiments, the combination therapy results in an overall survival which is increased in the treated subject compared to an antibody-drug conjugate or a binding agent (e.g. bispecific antibody) treatment alone. In some embodiments, the combination therapy results in a median progression-free survival which is increased in the treated subject compared to an antibody-drug conjugate or a binding agent (e.g. bispecific antibody) treatment alone.

In some embodiments, the cancer is positive for PD-L1.

In some embodiments, PD-L1 is expressed at least on a portion of the cells of the cancer, such as at least 10%, at least 20%, at least 30%, at least 40%, or at least 50%.

In some embodiments, PD-L1 is expressed on the surface of at least a portion of the tumour cells of the solid tumour, such as at least 10%, at least 20%, at least 30%, at least 40%, or at least 50%.

In some embodiments, PD-L1 is expressed on the surface of at least a portion of the tumour cells of the solid tumour and one or more target expressed on the surface of the tumour cells as described herein is/are expressed (i) a different portion, (ii) a same portion or (iii) an overlapping portion of the tumour cells of the solid tumour.

In some embodiments, the cancer is positive for one or more targets expressed on the surface of a tumour cell. In some embodiments the cancer comprises at least some cells that express on their surface the target that is specifically bound by the antibody or fragment thereof comprised in the antibody-drug conjugate. This may be determined by any method known in the art, for example using immunohistochemistry. Example targets are described herein, and may include B7H3, TROP2, HER2, HER3, ALK receptor (pleiotrophin receptor); ovarian carcinoma antigen (CA125); prostate-specific antigen (PSA); melanoma-associated antigen p97; melanoma antigen gp75; high molecular weight melanoma antigen (HMW-MAA); prostate-specific membrane antigen (PSMA); carcinoembryonic antigen (CEA); polymorphic epithelial mucin antigen; CD19; human B-lymphoma antigen-CD20; CD33; melanoma-specific antigens such as ganglioside GD2, ganglioside GD3, ganglioside GM2, and ganglioside GM3; epidermal growth factor receptor (EGFR); NY—BR-16; NY—BR-16; polymorphic epithelial mucin (PEM); malignant human lymphocyte antigen-APO-1; SSEA-1; SCP-1; SSEA-3; SSEA-4; sialyl Tn (STn); colon cancer antigen NY—CO-45; lung cancer antigen NY-LU-12 variant A; adenocarcinoma antigen ART1; Neuro-oncological ventral antigen 2 (NOVA2); tumor-associated antigens MAGE-C1 (cancer/testis antigen CT7), MAGE-B1 (MAGE-XP antigen), MAGE-B2 (DAM6), MAGE-2, MAGE-4a, MAGE-4b and MAGE-X2; Cancer-Testis Antigen (NY-EOS-1); YKL-40; EpCAM; EREG; CA15-3; sialyl SSEA-1(SLX); HER3; CLDN6; CLDN18.2, Mesothelin and any combination(s) thereof. Other example targets may include CD30, Nectin-4, TF, B7-H4 and ITGB6 and any combination(s) thereof.

In some embodiments, the cancer comprises at least some cells that express on their surface B7H3, TROP2, HER2, or any combination(s) thereof. In some embodiments, the cancer comprises at least some cells that express on their surface B7H3. In some embodiments, the cancer comprises at least some cells that express on their surface TROP2. In some embodiments, the cancer comprises at least some cells that express on their surface HER2. Other targets may additionally be expressed on the surface of the cells, such as PD-L1.

In some embodiments, the cancer is a B7H3-positive cancer, a TROP2-positive cancer, or a HER2-positive cancer, or any combination(s) thereof. In some embodiments, the cancer is a B7H3-positive cancer. In some embodiments, the cancer is a TROP2-positive cancer. In some embodiments, the cancer is a HER2-positive cancer. In some embodiments, the cancer is a PD-L1-positive cancer. In some embodiments, the cancer is B7H3-positive and PD-L1-positive. In some embodiments, the cancer is TROP2-positive and PD-L1-positive. In some embodiments, the cancer is HER2-positive and PD-L1-positive.

In some embodiments, the cancer comprises at least some cells that express on their surface CD30, Nectin-4, TF, B7-H4, ITGB6, HER2, or any combination(s) thereof. In some embodiments, the cancer comprises at least some cells that express on their surface CD30. In some embodiments, the cancer comprises at least some cells that express on their surface Nectin-4. In some embodiments, the cancer comprises at least some cells that express on their surface TF. In some embodiments, the cancer comprises at least some cells that express on their surface B7-H4. In some embodiments, the cancer comprises at least some cells that express on their surface ITGB6. In some embodiments, the cancer comprises at least some cells that express on their surface HER2. Other targets may additionally be expressed on the surface of the cells, such as PD-L1.

In some embodiments, the cancer is a CD30-positive cancer, a Nectin-4-positive cancer, a TF-positive cancer, a B7-H4-positive cancer, a ITGB6-positive cancer, or a HER2-positive cancer, or any combination(s) thereof. In some embodiments, the cancer is a CD30-positive cancer. In some embodiments, the cancer is a Nectin-4-positive cancer. In some embodiments, the cancer is a TF-positive cancer. In some embodiments, the cancer is a B7-H4-positive cancer. In some embodiments, the cancer is a ITGB6-positive cancer. In some embodiments, the cancer is a HER2-positive cancer. In some embodiments, the cancer is a PD-L1-positive cancer. In some embodiments, the cancer is CD30-positive and PD-L1-positive. In some embodiments, the cancer is Nectin-4-positive and PD-L1-positive. In some embodiments, the cancer is TF-positive and PD-L1-positive. In some embodiments, the cancer is B7-H4-positive and PD-L1-positive. In some embodiments, the cancer is ITGB6-positive and PD-L1-positive. In some embodiments, the cancer is HER2-positive and PD-L1-positive.

In some preferred embodiments, the cancer is a solid tumour. In some embodiments, the solid tumour expresses one or more targets described herein.

In some embodiments, the solid tumour expresses (a) one or more targets described herein, or any combination(s) thereof; and (b) expresses or over-expresses PD-L1. In some embodiments, at least 10%, at least 20%, at least 30%, at least 40%, or at least 50% of the tumour cells (over)-express one or more targets described herein. In some embodiments, at least 10%, at least 20%, at least 30%, at least 40%, or at least 50% of the tumour cells (over)-express PD-L1. In some embodiments, a target is over-expressed if the expression of the target on the tumour cells or in the solid tumour is at least 10%, at least 20%, at least 30%, at least 40%, or at least 50% higher than in a wild-type or surrounding healthy cell, e.g., non-tumour cell, preferably of the same cell type as the tumour cell, or tissues.

In some embodiments, the solid tumour expresses the target B7H3, TROP2, HER2, or any combination(s) thereof. In some embodiments, the solid tumour expresses PD-L1. In some embodiments, the solid tumour expresses (a) B7H3, TROP2, HER2, or any combination(s) thereof, and (b) PD-L1. In some embodiments, the solid tumour over-expresses one or more targets described herein. In some embodiments, the solid tumour over-expresses B7H3, TROP2, HER2, or any combination(s) thereof. In some embodiments, the solid tumour over-expresses PD-L1. In some embodiments, the solid tumour over-expresses (a) B7H3, TROP2, HER2, or any combination(s) thereof, and (b) PD-L1. In some embodiments, at least 10%, at least 20%, at least 30%, at least 40%, or at least 50% of the tumour cells (over)-express one or more targets described herein. In some embodiments, at least 10%, at least 20%, at least 30%, at least 40%, or at least 50% of the tumour cells (over)-express B7H3, TROP2, HER2, or any combination(s) thereof and/or PD-L1. In some embodiments, a target is over-expressed if the expression of the target on the tumour cells or in the solid tumour is at least 10%, at least 20%, at least 30%, at least 40%, or at least 50% higher than in a wildtype or surrounding healthy cell, e.g., non-tumour cell, preferably of the same cell type as the tumour cell, or tissues.

In some embodiments, the solid tumour expresses the target CD30, Nectin-4, TF, B7-H4, ITGB6, HER2, or any combination(s) thereof. In some embodiments, the solid tumour expresses PD-L1. In some embodiments, the solid tumour expresses (a) CD30, Nectin-4, TF, B7-H4, ITGB6, HER2, or any combination(s) thereof; and (b) PD-L1. In some embodiments, the solid tumour over-expresses one or more targets described herein. In some embodiments, the solid tumour over-expresses CD30, Nectin-4, TF, B7-H4, ITGB6, HER2, or any combination(s) thereof. In some embodiments, the solid tumour over-expresses PD-L1. In some embodiments, the solid tumour over-expresses (a) CD30, Nectin-4, TF, B7-H4, ITGB6, HER2, or any combination(s) thereof; and (b) PD-L1. In some embodiments, at least 10%, at least 20%, at least 30%, at least 40%, or at least 50% of the tumour cells (over)-express one or more targets described herein. In some embodiments, at least 10%, at least 20%, at least 30%, at least 40%, or at least 50% of the tumour cells (over)-express CD30, Nectin-4, TF, B7-H4, ITGB6, HER2, or any combination(s) thereof and/or PD-L1. In some embodiments, a target is over-expressed if the expression of the target on the tumour cells or in the solid tumour is at least 10%, at least 20%, at least 30%, at least 40%, or at least 50% higher than in a wildtype or surrounding healthy cell, e.g., non-tumour cell, preferably of the same cell type as the tumour cell, or tissues.

In some embodiments, the cancer is selected from esophageal cancer (e.g., esophageal adenocarcinoma and esophageal squamous cell carcinoma), brain tumour, lung cancer, small cell lung cancer (SCLC), non-small cell lung cancer (NSCLC) with or without actionable oncogenic alterations (AGA) (e.g., small cell lung cancer and non-small cell lung cancer), squamous cell carcinoma, bladder cancer, stomach cancer, ovarian cancer, peritoneal cancer, pancreatic cancer, breast cancer, triple-negative breast cancer (TNBC), head and neck cancer, cervical cancer, endometrial cancer, gastro-intestinal cancer, colorectal cancer, liver cancer, kidney cancer, urothelial cancer, solid tumour, non-Hodgkin lymphoma, central nervous system tumour (e.g., neuroglioma, glioblastoma multiforme, glioma or sarcoma), adenocarcinoma, prostate cancer, thyroid cancer, soft tissue sarcoma, vulvar cancer, or gallbladder cancer.

In some embodiments, the cancer is selected from esophageal cancer (e.g., esophageal adenocarcinoma and esophageal squamous cell carcinoma), brain tumour, lung cancer, small cell lung cancer (SCLC), non-small cell lung cancer (NSCLC) with or without actionable oncogenic alterations (AGA) (e.g., small cell lung cancer and non-small cell lung cancer), squamous cell carcinoma, bladder cancer, stomach cancer, ovarian cancer, peritoneal cancer, pancreatic cancer, breast cancer, triple-negative breast cancer (TNBC), head and neck cancer, cervical cancer, endometrial cancer, gastro-intestinal cancer, colorectal cancer, liver cancer, kidney cancer, urothelial cancer, solid tumour, non-Hodgkin lymphoma, central nervous system tumour (e.g., neuroglioma, glioblastoma multiforme, glioma or sarcoma), adenocarcinoma, prostate cancer or thyroid cancer.

In some embodiments, the cancer is selected from colorectal cancer (e.g., colon cancer), lung cancer, (e.g. small cell lung cancer (SCLC), non-small cell lung cancer (NSCLC) with or without actionable oncogenic alterations (AGA)), breast cancer, (e.g. triple-negative breast cancer (TNBC)), prostate cancer, soft tissue sarcoma, vulvar cancer, and gallbladder cancer.

In some embodiments, the cancer is selected from colorectal cancer (e.g., colon cancer), lung cancer, (e.g. small cell lung cancer (SCLC), non-small cell lung cancer (NSCLC) with or without actionable oncogenic alterations (AGA)), breast cancer, (e.g. triple-negative breast cancer (TNBC)), and prostate cancer.

In some embodiments, the cancer is selected from lung cancer, (e.g. small cell lung cancer (SCLC), non-small cell lung cancer (NSCLC) with or without actionable oncogenic alterations (AGA)), kidney cancer, urinary tract carcinoma, colorectal cancer, prostatic cancer, glioblastoma multiforme, ovarian cancer, pancreatic cancer, breast cancer, (e.g. triple-negative breast cancer (TNBC)), melanoma, liver cancer, bladder cancer, stomach cancer, esophageal cancer, soft tissue sarcoma, vulvar cancer and gallbladder cancer.

In some embodiments, the cancer is selected from lung cancer, (e.g. small cell lung cancer (SCLC), non-small cell lung cancer (NSCLC) with or without actionable oncogenic alterations (AGA)), kidney cancer, urinary tract carcinoma, colorectal cancer, prostatic cancer, glioblastoma multiforme, ovarian cancer, pancreatic cancer, breast cancer, (e.g. triple-negative breast cancer (TNBC)), melanoma, liver cancer, bladder cancer, stomach cancer and esophageal cancer.

Exemplary cancer include but are not limited to non-small-cell lung cancer (NSCLC), small cell lung cancer (SCLC), triple negative breast cancer (TNBC), hormone receptor (HR)-positive human epidermal growth factor receptor 2 (HER2)-negative breast cancer (HR+/HER2-BC), endometrial carcinoma (EC), pancreatic adenocarcinoma, gastric/gastroesophageal junction (GEJ) cancer, esophageal cancer, head and neck squamous cell carcinoma (HNSCC), transitional cell carcinoma of the urothelium, colorectal cancer (CRC), ovarian cancer (OC), cervical cancer (CC), and castration-resistant prostate cancer (CRPC).

In some embodiments, the cancer is selected from breast cancer, lung cancer, gastro-intestinal cancer, and adenocarcinoma. In some embodiments, the cancer is breast cancer, lung cancer, gastro-intestinal cancer, or adenocarcinoma. In some embodiments, the cancer is breast cancer such as non-small cell lung cancer (NSCLC). In some embodiments, the cancer is lung cancer such as triple negative breast cancer (TBNC). In some embodiments, the cancer is gastro-intestinal cancer. In some embodiments, the cancer is adenocarcinoma.

In some embodiments, the cancer is soft tissue sarcoma, vulvar cancer, or gallbladder cancer.

In some embodiments, the cancer is soft tissue sarcoma, vulvar cancer, or gallbladder cancer and the combination comprises a. a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction, and b. an antibody-drug conjugate comprising an antibody or fragment thereof that specifically binds to TROP2.

In some embodiments, the cancer is soft tissue sarcoma, vulvar cancer, or gallbladder cancer and the combination comprises a. a bispecific antibody that comprises (a) an antibody or fragment thereof that specifically binds to PD-L1 and (b) an antibody or fragment thereof that specifically binds to VEGF, and b. an antibody-drug conjugate comprising an antibody or fragment thereof that specifically binds to TROP2.

In some embodiments, the cancer is soft tissue sarcoma, vulvar cancer, or gallbladder cancer and the combination comprises Compound 5 and Compound 3.

### Antibody-Drug Conjugate Compositions

The present invention also provides a composition which comprises one or more antibody-drug conjugates as defined herein. The composition may be used in any of the combinations of the invention and uses as disclosed herein.

Features described herein in more detail among others for the “antibody-drug conjugate components” embodiments, the “linker and payload moieties” and the “linker-payload” embodiments equally apply to the “antibody-drug conjugate composition” embodiments which contain mixtures of the antibody-drug conjugates disclosed herein.

As indicated above, the drug-antibody ratio (DAR) of the antibody-drug conjugates as described herein may vary. Consequently, the composition may comprise a mixture of antibody-drug conjugates having a number of different DARs, and may therefore have an average DAR which is non-integral. In this specification “average DAR” and “average connection number” are synonymous.

In some embodiments, the average DAR is an integer or decimal from about 1 to about 16. In some embodiments, the average DAR is an integer or decimal of about 1, about 2, about 3, about 4, about 5, about 6, about 7, about 8, about 9, about 10, about 11, about 12, about 13, about 14, about 15 or about 16. In some embodiments, the average DAR is an integer or decimal from about 2 to about 14. In some embodiments, the average DAR is an integer or decimal from about 4 to about 12. In some embodiments, the average DAR is an integer or decimal from about 6 to about 10. In some embodiments, the average DAR is an integer or decimal from about 7 to about 9. In some embodiments, the average DAR is an integer or decimal from about 7.5 to about 8.5. In some embodiments, the average DAR is an integer or decimal from about 7.8 to about 8.2.

In some embodiments, the average DAR is about 3.0. In some embodiments, the average DAR is about 3.1. In some embodiments, the average DAR is about 3.2. In some embodiments, the average DAR is about 3.3. In some embodiments, the average DAR is about 3.4. In some embodiments, the average DAR is about 3.5. In some embodiments, the average DAR is about 3.6. In some embodiments, the average DAR is about 3.7. In some embodiments, the average DAR is about 3.8. In some embodiments, the average DAR is about 3.9. In some embodiments, the average DAR is about 4.0. In some embodiments, the average DAR is about 4.1. In some embodiments, the average DAR is about 4.2. In some embodiments, the average DAR is about 4.3. In some embodiments, the average DAR is about 4.4. In some embodiments, the average DAR is about 4.5. In some embodiments, the average DAR is about 4.6. In some embodiments, the average DAR is about 4.7. In some embodiments, the average DAR is about 4.8. In some embodiments, the average DAR is about 4.9. In some embodiments, the average DAR is about 5.0. In some embodiments, the average DAR is about 5.1. In some embodiments, the average DAR is about 5.2. In some embodiments, the average DAR is about 5.3. In some embodiments, the average DAR is about 5.4. In some embodiments, the average DAR is about 5.5. In some embodiments, the average DAR is about 5.6. In some embodiments, the average DAR is about 5.7. In some embodiments, the average DAR is about 5.8. In some embodiments, the average DAR is about 5.9. In some embodiments, the average DAR is about 6.0. In some embodiments, the average DAR is about 6.1. In some embodiments, the average DAR is about 6.2. In some embodiments, the average DAR is about 6.3. In some embodiments, the average DAR is about 6.4. In some embodiments, the average DAR is about 6.5. In some embodiments, the average DAR is about 6.6. In some embodiments, the average DAR is about 6.7. In some embodiments, the average DAR is about 6.8. In some embodiments, the average DAR is about 6.9. In some embodiments, the average DAR is about 7.0. In some embodiments, the average DAR is about 7.1. In some embodiments, the average DAR is about 7.2. In some embodiments, the average DAR is about 7.3. In some embodiments, the average DAR is about 7.4. In some embodiments, the average DAR is about 7.5. In some embodiments, the average DAR is about 7.6. In some embodiments, the average DAR is about 7.7. In some embodiments, the average DAR is about 7.8. In some embodiments, the average DAR is about 7.9. In some embodiments, the average DAR is about 8.0. In some embodiments, the average DAR is about 8.1. In some embodiments, the average DAR is about 8.2. In some embodiments, the average DAR is about 8.3. In some embodiments, the average DAR is about 8.4. In some embodiments, the average DAR is about 8.5. In some embodiments, the average DAR is about 8.6. In some embodiments, the average DAR is about 8.7. In some embodiments, the average DAR is about 8.8. In some embodiments, the average DAR is about 8.9. In some embodiments, the average DAR is about 9.0. In some embodiments, the average DAR is about 9.1. In some embodiments, the average DAR is about 9.2. In some embodiments, the average DAR is about 9.3. In some embodiments, the average DAR is about 9.4. In some embodiments, the average DAR is about 9.5. In some embodiments, the average DAR is about 9.6. In some embodiments, the average DAR is about 9.7. In some embodiments, the average DAR is about 9.8. In some embodiments, the average DAR is about 9.9. In some embodiments, the DAR is about 10.0.

In some embodiments, the composition comprises one or more antibody-drug conjugates having the structure shown as formula (III-D′):

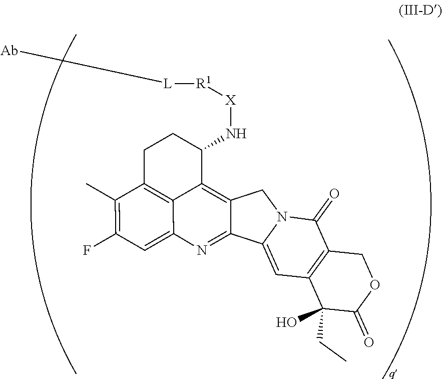

wherein:
Ab, L, R1 and X are as defined above for formula (III-D), either in its broadest aspect or a preferred aspect; and
q′ is an average connection number and is an integer or decimal from 1 to 16. In some embodiments, q′ is an integer or decimal from about 2 to about 14. In some embodiments, q′ is an integer or decimal from about 4 to about 12. In some embodiments, q′ is an integer or decimal from about 6 to about 10.

In some embodiments, the composition comprises one or more conjugates having the structure shown as Formula (IVA′):

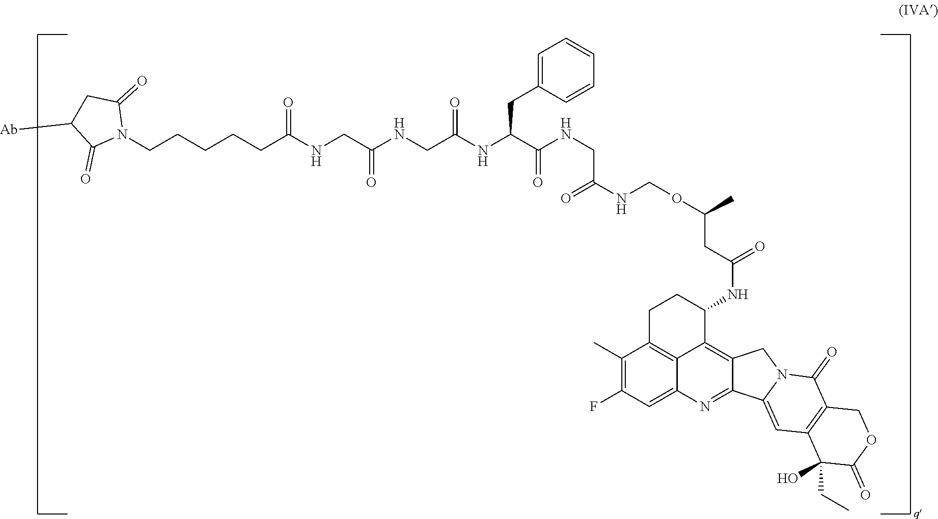

or a pharmaceutically acceptable salt thereof, wherein,
q′ represents an average connection number, and is an integer or decimal from 1 to 16, and Ab is an antibody or fragment thereof as defined herein. In some embodiments, q′ is an integer or decimal from about 2 to about 14. In some embodiments, q′ is an integer or decimal from about 4 to about 12. In some embodiments, q′ is an integer or decimal from about 6 to about 10.

In some embodiments, the composition comprises one or more conjugates having the structure shown as formula (II-Dx′):

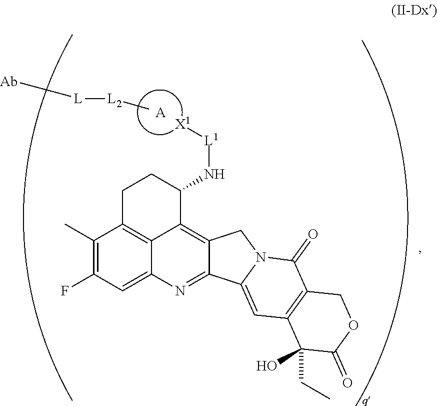

wherein:
Ab, L, L2, ring A, X and L1 are as defined above for formula (III-D), either in its broadest aspect or a preferred aspect; and
q′ represents an average connection number, and is an integer or decimal from 1 to 16. In some embodiments, q′ is an integer or decimal from about 2 to about 14. In some embodiments, q′ is an integer or decimal from about 4 to about 12. In some embodiments, q′ is an integer or decimal from about 6 to about 10.

In some embodiments, the composition comprises one or more conjugates having the structure shown as formula (IVB′)

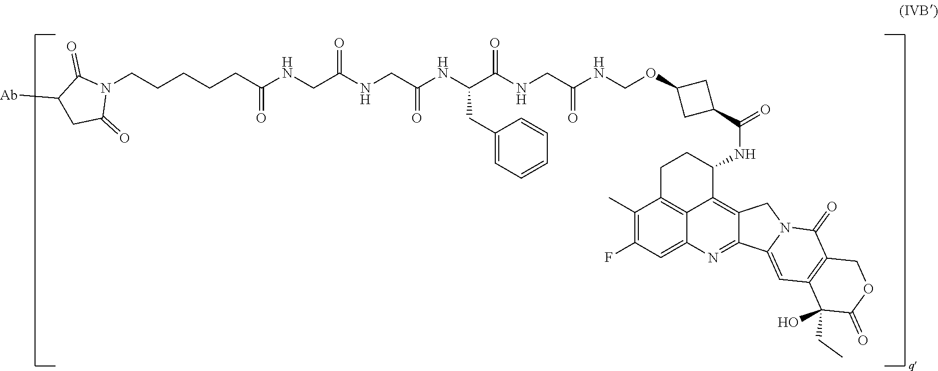

or a pharmaceutically acceptable salt thereof, wherein,
q′ represents an average connection number, and is an integer or decimal from 1 to 16; and Ab is an antibody or fragment thereof as defined herein.

In some embodiments, q′ is an integer or decimal from about 2 to about 14. In some embodiments, q′ is an integer or decimal from about 4 to about 12. In some embodiments, q′ is an integer or decimal from about 6 to about 10.

In some embodiments of formulae (III-D′), (IVA′), (II-Dx′) or (IVB′), the average connection number q′ is an integer or decimal from about 1 to about 16. In some embodiments of formulae (III-D′), (IVA′), (II-Dx′) or (IVB′), the average connection number q′ is an integer or decimal of about 1, about 2, about 3, about 4, about 5, about 6, about 7, about 8, about 9, about 10, about 11, about 12, about 13, about 14, about 15 or about 16. In some embodiments of formulae (III-D′), (IVA′), (II-Dx′) or (IVB′), the average connection number q′ is an integer or decimal from about 4 to about 12. In some embodiments of formulae (III-D′), (IVA′), (II-Dx′) or (IVB′), the average connection number q′ is an integer or decimal from about 6 to about 10. In some embodiments of formulae (III-D′), (IVA′), (II-Dx′) or (IVB′), the average connection number q′ is an integer or decimal from about 7 to about 9. In some embodiments of formulae (III-D′), (IVA′), (II-Dx′) or (IVB′), the average connection number q′ is from about 7.5 to about 8.5. In some embodiments of formulae (III-D′), (IVA′), (II-Dx′) or (IVB′), the average connection number q′ is from about 7.8 to about 8.2.

In some embodiments of formulae (III-D′), (IVA′), (II-Dx′) or (IVB′), the average connection number q′ is about 3.0. In some embodiments of formulae (III-D′), (IVA′), (II-Dx′) or (IVB′), the average connection number q′ is about 3.1. In some embodiments of formulae (III-D′), (IVA′), (II-Dx′) or (IVB′), the average connection number q′ is about 3.2. In some embodiments of formulae (III-D′), (IVA′), (II-Dx′) or (IVB′), the average connection number q′ is about 3.3. In some embodiments of formulae (III-D′), (IVA′), (II-Dx′) or (IVB′), the average connection number q′ is about 3.4. In some embodiments of formulae (III-D′), (IVA′), (II-Dx′) or (IVB′), the average connection number q′ is about 3.5. In some embodiments of formulae (III-D′), (IVA′), (II-Dx′) or (IVB′), the average connection number q′ is about 3.6. In some embodiments of formulae (III-D′), (IVA′), (II-Dx′) or (IVB′), the average connection number q′ is about 3.7. In some embodiments of formulae (III-D′), (IVA′), (II-Dx′) or (IVB′), the average connection number q′ is about 3.8. In some embodiments of formulae (III-D′), (IVA′), (II-Dx′) or (IVB′), the average connection number q′ is about 3.9. In some embodiments of formulae (III-D′), (IVA′), (II-Dx′) or (IVB′), the average connection number q′ is about 4.0. In some embodiments of formulae (III-D′), (IVA′), (II-Dx′) or (IVB′), the average connection number q′ is about 4.1. In some embodiments of formulae (III-D′), (IVA′), (II-Dx′) or (IVB′), the average connection number q′ is about 4.2. In some embodiments of formulae (III-D′), (IVA′), (II-Dx′) or (IVB′), the average connection number q′ is about 4.3. In some embodiments of formulae (III-D′), (IVA′), (II-Dx′) or (IVB′), the average connection number q′ is about 4.4. In some embodiments of formulae (III-D′), (IVA′), (II-Dx′) or (IVB′), the average connection number q′ is about 4.5. In some embodiments of formulae (III-D′), (IVA′), (II-Dx′) or (IVB′), the average connection number q′ is about 4.6. In some embodiments of formulae (III-D′), (IVA′), (II-Dx′) or (IVB′), the average connection number q′ is about 4.7. In some embodiments of formulae (III-D′), (IVA′), (II-Dx′) or (IVB′), the average connection number q′ is about 4.8. In some embodiments of formulae (III-D′), (IVA′), (II-Dx′) or (IVB′), the average connection number q′ is about 4.9. In some embodiments of formulae (III-D′), (IVA′), (II-Dx′) or (IVB′), the average connection number q′ is about 5.0. In some embodiments of formulae (III-D′), (IVA′), (II-Dx′) or (IVB′), the average connection number q′ is about 5.1. In some embodiments of formulae (III-D′), (IVA′), (II-Dx′) or (IVB′), the average connection number q′ is about 5.2. In some embodiments of formulae (III-D′), (IVA′), (II-Dx′) or (IVB′), the average connection number q′ is about 5.3. In some embodiments of formulae (III-D′), (IVA′), (II-Dx′) or (IVB′), the average connection number q′ is about 5.4. In some embodiments of formulae (III-D′), (IVA′), (II-Dx′) or (IVB′), the average connection number q′ is about 5.5. In some embodiments of formulae (III-D′), (IVA′), (II-Dx′) or (IVB′), the average connection number q′ is about 5.6. In some embodiments of formulae (III-D′), (IVA′), (II-Dx′) or (IVB′), the average connection number q′ is about 5.7. In some embodiments of formulae (III-D′), (IVA′), (II-Dx′) or (IVB′), the average connection number q′ is about 5.8. In some embodiments of formulae (III-D′), (IVA′), (II-Dx′) or (IVB′), the average connection number q′ is about 5.9. In some embodiments of formulae (III-D′), (IVA′), (II-Dx′) or (IVB′), the average connection number q′ is about 6.0. In some embodiments of formulae (III-D′), (IVA′), (II-Dx′) or (IVB′), the average connection number q′ is about 6.1. In some embodiments of formulae (III-D′), (IVA′), (II-Dx′) or (IVB′), the average connection number q′ is about 6.2. In some embodiments of formulae (III-D′), (IVA′), (II-Dx′) or (IVB′), the average connection number q′ is about 6.3. In some embodiments of formulae (III-D′), (IVA′), (II-Dx′) or (IVB′), the average connection number q′ is about 6.4. In some embodiments of formulae (III-D′), (IVA′), (II-Dx′) or (IVB′), the average connection number q′ is about 6.5. In some embodiments of formulae (III-D′), (IVA′), (II-Dx′) or (IVB′), the average connection number q′ is about 6.6. In some embodiments of formulae (III-D′), (IVA′), (II-Dx′) or (IVB′), the average connection number q′ is about 6.7. In some embodiments of formulae (III-D′), (IVA′), (II-Dx′) or (IVB′), the average connection number q′ is about 6.8. In some embodiments of formulae (III-D′), (IVA′), (II-Dx′) or (IVB′), the average connection number q′ is about 6.9. In some embodiments of formulae (III-D′) (IVA′), (II-Dx′) or (IVB′), the average connection number q′ is about 7.0. In some embodiments of formulae (III-D′), (IVA′), (II-Dx′) or (IVB′), the average connection number q′ is about 7.1. In some embodiments of formulae (III-D′), (IVA′), (II-Dx′) or (IVB′), the average connection number q′ is about 7.2. In some embodiments of formulae (III-D′), (IVA′), (II-Dx′) or (IVB′), the average connection number q′ is about 7.3. In some embodiments of formulae (III-D′), (IVA′), (II-Dx′) or (IVB′), the average connection number q′ is about 7.4. In some embodiments of formulae (III-D′), (IVA′), (II-Dx′) or (IVB′), the average connection number q′ is about 7.5. In some embodiments of formulae (III-D′), (IVA′), (II-Dx′) or (IVB′), the average connection number q′ is about 7.6. In some embodiments of formulae (III-D′), (IVA′), (II-Dx′) or (IVB′), the average connection number q′ is about 7.7. In some embodiments of formulae (III-D′), (IVA′), (II-Dx′) or (IVB′), the average connection number q′ is about 7.8. In some embodiments of formulae (III-D′), (IVA′), (II-Dx′) or (IVB′), the average connection number q′ is about 7.9. In some embodiments of formulae (III-D′), (IVA′), (II-Dx′) or (IVB′), the average connection number q′ is about 8.0. In some embodiments of formulae (III-D′), (IVA′), (II-Dx′) or (IVB′), the average connection number q′ is about 8.1. In some embodiments of formulae (III-D′), (IVA′), (II-Dx′) or (IVB′), the average connection number q′ is about 8.2. In some embodiments of formulae (III-D′), (IVA′), (II-Dx′) or (IVB′), the average connection number q′ is about 8.3. In some embodiments of formulae (III-D′), (IVA′), (II-Dx′) or (IVB′), the average connection number q′ is about 8.4. In some embodiments of formulae (III-D′), (IVA′), (II-Dx′) or (IVB′), the average connection number q′ is about 8.5. In some embodiments of formulae (III-D′), (IVA′), (II-Dx′) or (IVB′), the average connection number q′ is about 8.6. In some embodiments of formulae (III-D′), (IVA′), (II-Dx′) or (IVB′), the average connection number q′ is about 8.7. In some embodiments of formulae (III-D′), (IVA′), (II-Dx′) or (IVB′), the average connection number q′ is about 8.8. In some embodiments of formulae (III-D′), (IVA′), (II-Dx′) or (IVB′), the average connection number q′ is about 8.9. In some embodiments of formulae (III-D′), (IVA′), (II-Dx′) or (IVB′), the average connection number q′ is about 9.0. In some embodiments of formulae (III-D′), (IVA′), (II-Dx′) or (IVB′), the average connection number q′ is about 9.1. In some embodiments of formulae (III-D′), (IVA′), (II-Dx′) or (IVB′), the average connection number q′ is about 9.2. In some embodiments of formulae (III-D′), (IVA′), (II-Dx′) or (IVB′), the average connection number q′ is about 9.3. In some embodiments of formulae (III-D′), (IVA′), (II-Dx′) or (IVB′), the average connection number q′ is about 9.4. In some embodiments of formulae (III-D′), (IVA′), (II-Dx′) or (IVB′), the average connection number q′ is about 9.5. In some embodiments of formulae (III-D′), (IVA′), (II-Dx′) or (IVB′), the average connection number q′ is about 9.6. In some embodiments of formulae (III-D′), (IVA′), (II-Dx′) or (IVB′), the average connection number q′ is about 9.7. In some embodiments of formulae (III-D′), (IVA′), (II-Dx′) or (IVB′), the average connection number q′ is about 9.8. In some embodiments of formulae (III-D′), (IVA′), (II-Dx′) or (IVB′), the average connection number q′ is about 9.9. In some embodiments of formulae (III-D′), (IVA′), (II-Dx′) or (IVB′), the average connection number q′ is about 10.0.

In one embodiment of compositions containing antibody-drug conjugates of formula (IVB′), the average connection number q′ is from about 6.0 to about 10.0. In one embodiment of compositions containing antibody-drug conjugates of formula (IVB′), the average connection number q′ is from about 7.0 to about 9.0. In one embodiment of compositions containing antibody-drug conjugates of formula (IVB′), the average connection number q′ is from about 7.5 to about 8.5. In one embodiment of compositions containing antibody-drug conjugates of formula (IVB′), the average connection number q′ is from about 7.8 to about 8.2. In one embodiment of compositions containing antibody-drug conjugates of formula (IVB′), the average connection number q′ is from about 7.9 to about 8.1. In one embodiment of compositions containing antibody-drug conjugates of formula (IVB′), the average connection number q′ is about 8.0.

In one embodiment of compositions containing antibody-drug conjugates of formula (IVA′), the average connection number q′ is from about 2.0 to about 6.0. In one embodiment of compositions containing antibody-drug conjugates of formula (IVA′), the average connection number q′ is from about 3.0 to about 5.0. In one embodiment of compositions containing antibody-drug conjugates of formula (IVA′), the average connection number q′ is from about 3.5 to about 4.5. In one embodiment of compositions containing antibody-drug conjugates of formula (IVA′), the average connection number q′ is from about 3.8 to about 4.2. In one embodiment of compositions containing antibody-drug conjugates of formula (IVB′), the average connection number q′ is from about 3.9 to about 4.1. In one embodiment of compositions containing antibody-drug conjugates of formula (IVA′), the average connection number q′ is about 4.0.

In one embodiment of compositions containing antibody-drug conjugates of formula (IVA′), the average connection number q′ is from about 4.0 to about 8.0. In one embodiment of compositions containing antibody-drug conjugates of formula (IVA′), the average connection number q′ is from about 5.0 to about 7.0. In one embodiment of compositions containing antibody-drug conjugates of formula (IVA′), the average connection number q′ is from about 5.5 to about 6.5. In one embodiment of compositions containing antibody-drug conjugates of formula (IVA′), the average connection number q′ is from about 5.8 to about 6.2. In one embodiment of compositions containing antibody-drug conjugates of formula (IVB′), the average connection number q′ is from about 5.9 to about 6.1. In one embodiment of compositions containing antibody-drug conjugates of formula (IVA′), the average connection number q′ is about 6.0.

In one embodiment, the composition comprises one or more antibody-drug conjugates having the structure shown as formula (VIIb′):

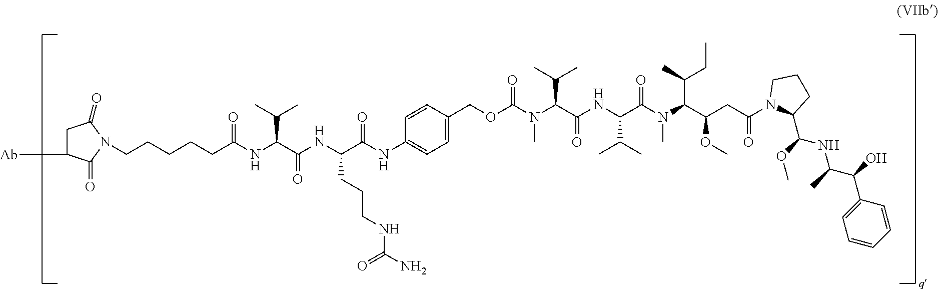

or a pharmaceutically acceptable salt thereof, wherein,
q′ represents an average connection number, and is an integer or decimal from ito 16; and
Ab is an antibody or fragment thereof as defined herein.

In some embodiments, Ab is an anti-CD30 antibody or fragment thereof, an anti-Nectin-4 antibody or fragment thereof, an anti-TF antibody or fragment thereof, an anti-B7-H4 antibody or fragment thereof, an anti-ITGB6 antibody or fragment thereof, or an anti-HER2 antibody or fragment thereof. In some embodiments, Ab is an anti-CD30 antibody, an anti-Nectin-4 antibody, an anti-TF antibody, an anti-B7-H4 antibody, an anti-ITGB6 antibody, or an anti-HER2 antibody.

In some embodiments, Ab is Brentuximab or a fragment thereof, Enfortumab or a fragment thereof, Tisotumab or a fragment thereof, Felmetatug or a fragment thereof, Sigvotatug or a fragment thereof, or Disitamab or a fragment thereof. In some embodiments, Ab is Brentuximab, Enfortumab, Tisotumab, Felmetatug, Sigvotatug, or Disitamab.

### Preferred Combinations

In one embodiment, the antibody-drug conjugate has the structural formula (II-T), as defined above, and the bispecific antibody is Compound 5, as defined herein.

In one embodiment, the antibody-drug conjugate is Compound 1, as defined herein, and the bispecific antibody is Compound 5, as defined herein.

In one embodiment, the antibody-drug conjugate has the structural formula (III-W), as defined above, and the bispecific antibody is Compound 5, as defined herein.

In one embodiment, the antibody-drug conjugate is Compound 2, as defined herein, and the bispecific antibody is Compound 5, as defined herein.

In one embodiment, the antibody-drug conjugate has the structural formula (III-S), as defined above, and the bispecific antibody is Compound 5, as defined herein.

In one embodiment, the antibody-drug conjugate is Compound 3, as defined herein, and the bispecific antibody is Compound 5, as defined herein.

In one embodiment, the antibody-drug conjugate has the structural formula (I-M), as defined above, and the bispecific antibody is Compound 5, as defined herein.

In one embodiment, the antibody-drug conjugate is Compound 4, as defined herein, and the bispecific antibody is Compound 5, as defined herein.

In one embodiment, the antibody-drug conjugate is of the formula (VIIb), as defined above, and the bispecific antibody is Compound 5, as defined herein.

In one embodiment, the antibody-drug conjugate is Compound 7, as defined herein, and the bispecific antibody is Compound 5, as defined herein.

In one embodiment, the antibody-drug conjugate is Compound 8, as defined herein, and the bispecific antibody is Compound 5, as defined herein.

In one embodiment, the antibody-drug conjugate is Compound 9, as defined herein, and the bispecific antibody is Compound 5, as defined herein.

In one embodiment, the antibody-drug conjugate is Compound 10, as defined herein, and the bispecific antibody is Compound 5, as defined herein.

In one embodiment, the antibody-drug conjugate is Compound 11, as defined herein, and the bispecific antibody is Compound 5, as defined herein.

In one embodiment, the antibody-drug conjugate is Compound 12, as defined herein, and the bispecific antibody is Compound 5, as defined herein.

In one embodiment, the antibody-drug conjugate has the structural formula (II-T), as defined above, and the bispecific antibody is Compound 13, as defined herein.

In one embodiment, the antibody-drug conjugate is Compound 1, as defined herein, and the bispecific antibody is Compound 13, as defined herein.

In one embodiment, the antibody-drug conjugate has the structural formula (III-W), as defined above, and the bispecific antibody is Compound 13, as defined herein.

In one embodiment, the antibody-drug conjugate is Compound 2, as defined herein, and the bispecific antibody is Compound 13, as defined herein.

In one embodiment, the antibody-drug conjugate has the structural formula (III-S), as defined above, and the bispecific antibody is Compound 13, as defined herein.

In one embodiment, the antibody-drug conjugate is Compound 3, as defined herein, and the bispecific antibody is Compound 13, as defined herein.

In one embodiment, the antibody-drug conjugate has the structural formula (I-M), as defined above, and the bispecific antibody is Compound 13, as defined herein.

In one embodiment, the antibody-drug conjugate is Compound 4, as defined herein, and the bispecific antibody is Compound 13, as defined herein.

In one embodiment, the antibody-drug conjugate is of the formula (VIIb), as defined above, and the bispecific antibody is Compound 13, as defined herein.

In one embodiment, the antibody-drug conjugate is Compound 7, as defined herein, and the bispecific antibody is Compound 13, as defined herein.

In one embodiment, the antibody-drug conjugate is Compound 8, as defined herein, and the bispecific antibody is Compound 13, as defined herein.

In one embodiment, the antibody-drug conjugate is Compound 9, as defined herein, and the bispecific antibody is Compound 13, as defined herein.

In one embodiment, the antibody-drug conjugate is Compound 10, as defined herein, and the bispecific antibody is Compound 13, as defined herein.

In one embodiment, the antibody-drug conjugate is Compound 11, as defined herein, and the bispecific antibody is Compound 13, as defined herein.

In one embodiment, the antibody-drug conjugate is Compound 12, as defined herein, and the bispecific antibody is Compound 13, as defined herein.

### Methods, Compositions, and Kits

In some aspects of the invention, a method of treating cancer in a subject is provided, the method comprising administering to the subject:
a. a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction; and
b. an antibody-drug conjugate comprising an antibody or fragment thereof that specifically binds to a target expressed on the surface of a tumour cell.

In some aspects of the invention, a method of treating cancer in a subject is provided, the method comprising administering to the subject:
a. a bispecific antibody that comprises:
(a) an antibody or fragment thereof that specifically binds to PD-L1, and
(b) an antibody or fragment thereof that specifically binds to VEGF; and b. an antibody-drug conjugate comprising an antibody or fragment thereof that specifically binds to a target expressed on the surface of a tumour cell.

The embodiments for the combination therapy disclosed herein comprising the binding agent (e.g. the bispecific antibody) in combination with the antibody-drug conjugate can be used in the method of treating cancer disclosed herein. Features described herein in more detail among others for the “binding agent” embodiments (e.g. the “bispecific antibody” embodiments), the “antibody-drug conjugate components” embodiments, the “linker and payload moieties” embodiments, the “linker-payload” embodiments and the “antibody-drug conjugate composition” embodiments equally apply to the method of treating cancer embodiments which may comprise administering the binding agent (e.g. the bispecific antibody) and antibody-drug conjugates disclosed herein to a subject.

In some aspects of the disclosure, a medical preparation or composition is provided comprising:
a. a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction; and
b. an antibody-drug conjugate comprising an antibody or fragment thereof that specifically binds to a target expressed on the surface of a tumour cell.

In some aspects of the disclosure, a medical preparation or composition is provided comprising:
a. a bispecific antibody that comprises:
(a) an antibody or fragment thereof that specifically binds to PD-L1, and
(b) an antibody or fragment thereof that specifically binds to VEGF; and
b. an antibody-drug conjugate comprising an antibody or fragment thereof that specifically binds to a target expressed on the surface of a tumour cell.

The embodiments for the combination therapy disclosed herein comprising the binding agent (e.g. the bispecific antibody) in combination with the antibody-drug conjugate can be used in the medical preparation or composition disclosed herein. Features described herein in more detail among others for the “binding agent” embodiments (e.g. the “bispecific antibody” embodiments), the “antibody-drug conjugate components” embodiments, the “linker and payload moieties” embodiments, the “linker-payload” embodiments, and the “antibody-drug conjugate composition” embodiments equally apply to the medical preparation or composition embodiments disclosed herein which may contain mixtures of the binding agent (e.g. the bispecific antibody) and antibody-drug conjugates disclosed herein.

In some embodiments, the medical preparation or composition is a pharmaceutical composition. In these embodiments, the medical preparation or pharmaceutical composition may further comprise one or more pharmaceutically acceptable carriers, diluents and/or excipients.

A pharmaceutical composition or medical preparation comprising the ADC and the anti-PD-1/PD-L1xVEGF/VEGFR binding agent as described herein may be administered to a subject to elicit, preferably improve, an immune response against the ADC. A person skilled in the art will know that one of the principles of immunotherapy and vaccination is based on the fact that an immunoprotective reaction to a disease is produced by immunizing a subject with an antigen or an epitope, which is immunologically relevant with respect to the disease to be treated. Accordingly, pharmaceutical compositions or medical preparations described herein are applicable for inducing or enhancing an immune response. Pharmaceutical compositions or medical preparations described herein are thus useful in a prophylactic and/or therapeutic treatment of a disease involving an antigen or epitope, in particular cancer.

In some embodiments, the combination, medical preparation or composition of the aspects of the present disclosure, is a kit. In these embodiments, the ADC and the anti-PD-1/PD-L1xVEGF/VEGFR binding agent preferably are in separate vials or containers, which may further comprise one or more pharmaceutically acceptable carriers, diluents and/or excipients as described herein. In case the combination, medical preparation or composition further comprises one or more further therapeutic agents (other than the ADC described herein and other than the anti-PD-1/PD-L1xVEGF/VEGFR binding agent described herein), it is also preferred for the kit that the one or more further therapeutic agents are in separate vials or containers (i.e., that the ADC, the anti-PD-1/PD-L1xVEGF/VEGFR binding agent, and the one or more further therapeutic agents are in separate vials or containers, preferably the ADC, the anti-PD-1/PD-L1xVEGF/VEGFR binding agent, and the one or more further therapeutic agents are in separate vials or containers), wherein each vial or container may further comprise one or more pharmaceutically acceptable carriers, diluents and/or excipients as described herein.

The agents described herein may be administered in combinations, pharmaceutical compositions or medicaments and may be administered in the form of any suitable pharmaceutical composition, medical preparation or combination.

In some embodiments, the agents described herein (such as ADC, anti-PD-1/PD-L1xVEGF/VEGFR binding agent, etc.) may be administered in a pharmaceutical composition or medical preparation. In one embodiment, the pharmaceutical composition or medical preparation is for therapeutic or prophylactic treatments, e.g., for inducing an immune response, preferably against cancer, e.g., for treating or preventing cancer.

In some embodiments, the agents described herein (such as combination partner, anti-PD-1/PD-L1xVEGF/VEGFR binding agent, etc.) may be present (e.g., in a kit or medical preparation) and/or administered (e.g., as part of a treatment method) as a combination, e.g., each of the agents described herein is contained (e.g., in a kit or medical preparation) in separate form from the other agents (e.g., each of the agents described herein is in separate vials or containers of a kit or medical preparation) and/or each of the agents described herein is administered separately from the other agents (e.g., each of the agents described herein is administered in separate dose forms). In one embodiment, the combination is for therapeutic or prophylactic treatments, e.g., for inducing an immune response, preferably against cancer, e.g., for treating or preventing cancer. The embodiments described herein for the pharmaceutical compositions are likewise applicable to the separate dose forms of the combinations.

In some embodiments, the combination, medical preparation or composition is for administration to a human. In one embodiment, the human subject to whom the combination, medical preparation or composition is to be administered has received prior cancer treatment, e.g., treatment with a PD-1/PD-L1 inhibitor and/or a chemotherapeutic (such as a platinum compound). In one embodiment, the human subject to whom the combination, medical preparation or composition is to be administered has lung cancer (in particular, NSCLC, e.g., advanced unresectable Stage III or metastatic Stage IV NSCLC) and has received prior cancer treatment with a PD-1/PD-L1 inhibitor and a platinum compound (but preferably is naïve to VEGF or VEGFR targeted treatment).

In some embodiment, a kit of the invention comprises:
(a) a composition comprising a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction; and
(b) a composition comprising an antibody-drug conjugate comprising an antibody or fragment thereof that specifically binds to a target expressed on the surface of a tumour cell.

In an embodiment, each composition of the kit is a pharmaceutical composition.

Further, the invention also provides in an embodiment a kit of parts comprising the bispecific antibody that specifically binds to PD-L1 and VEGF and a chemotherapy agent.

A kit of the invention typically includes instructions.

The pharmaceutical compositions according to the present disclosure preferably comprise one or more adjuvants or may be administered with one or more adjuvants. Adjuvants comprise a heterogeneous group of compounds such as oil emulsions (e.g., Freund's adjuvants), mineral compounds (such as alum), bacterial products (such as Bordetella pertussis toxin), or immune-stimulating complexes. Examples of adjuvants include, without limitation, LPS, GP96, CpG oligodeoxynucleotides, growth factors, and cytokines, such as monokines, lymphokines, interleukins, chemokines. The chemokines may be IL-1, IL-2, IL-3, IL-4, IL-5, IL-6, IL-7, IL-8, IL-9, IL-10, IL-12, INF-α, INF-γ, GM-CSF, LT-a. Further known adjuvants are aluminum hydroxide, Freund's adjuvant or oil such as Montanide® ISA51. Other suitable adjuvants for use in the present disclosure include lipopeptides, such as Pam3Cys. The pharmaceutical compositions according to the present disclosure are generally applied in a therapeutically effective amount and in a pharmaceutically acceptable preparation.

In some embodiments, an effective amount comprises an amount sufficient to cause a tumor/lesion to shrink. In some embodiments, an effective amount is an amount sufficient to decrease the growth rate of a tumor (such as to suppress tumor growth). In some embodiments, an effective amount is an amount sufficient to delay tumor development. In some embodiments, an effective amount is an amount sufficient to prevent or delay tumor recurrence. In some embodiments, an effective amount is an amount sufficient to increase a subject's immune response to a tumor, such that tumor growth and/or size and/or metastasis is reduced, delayed, ameliorated, and/or prevented. An effective amount can be administered in one or more administrations. In some embodiments, administration of an effective amount (e.g., of a composition comprising the [combination partner] and/or the binding agent as described herein, e.g. the binding agent binding to (i) PD-1, PD-L1, or both and (ii) VEGF, VEGFR, or both) may: (i) reduce the number of cancer cells; (ii) reduce tumor size; (iii) inhibit, retard, slow to some extent and may stop cancer cell infiltration into peripheral organs; (iv) inhibit (e.g., slow to some extent and/or block or prevent) metastasis; (v) inhibit tumor growth; (vi) prevent or delay occurrence and/or recurrence of tumor; and/or (vii) relieve to some extent one or more of the symptoms associated with the cancer.

The pharmaceutical compositions according to the present disclosure may contain salts, buffers, preservatives, and optionally other therapeutic agents. In one embodiment, the pharmaceutical compositions of the present disclosure comprise one or more pharmaceutically acceptable carriers, diluents, and/or excipients. Suitable preservatives for use in the pharmaceutical compositions of the present disclosure include, without limitation, benzalkonium chloride, chlorobutanol, paraben, and thimerosal.

A (pharmaceutical) composition may also comprise pharmaceutically acceptable antioxidants for instance (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

A (pharmaceutical) composition may also comprise isotonicity agents, such as sugars, polyalcohols, such as mannitol, sorbitol, glycerol or sodium chloride in the composition.

In one embodiment, the (pharmaceutical) composition comprising the binding agent of the present invention comprises salt, buffers, amino acids, stabilizers, and surface-active agents. In one embodiment, the (pharmaceutical) composition comprising the binding agent of the present invention comprises no preservative. In a more specific embodiment, the (pharmaceutical) composition comprising the binding agent of the present invention comprises L-histidine, histidine hydrochloride, sucrose, disodium edetate, polysorbate 80 and water for injection. In a more specific embodiment, the (pharmaceutical) composition comprising the binding agent of the present invention is diluted with sodium chloride prior to administration.

In one embodiment, pharmaceutical compositions according to the present disclosure may be administered intravenously, intraarterially, subcutaneously, intradermally, intranodullary or intramuscularly. In certain embodiments, the pharmaceutical composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection or infusion. In a preferred embodiment, the pharmaceutical composition is formulated for systemic administration. In another preferred embodiment, the systemic administration is by intravenous administration.

Pharmaceutical compositions for injection or infusion must typically be sterile and stable under the conditions of manufacture and storage. The composition may be formulated as a solution, micro-emulsion, liposome, or other ordered structure suitable to high drug concentration. The carrier may be an aqueous or a non-aqueous solvent or dispersion medium containing for instance water, ethanol, polyols (such as glycerol, propylene glycol, polyethylene glycol, and the like), and suitable mixtures thereof, vegetable oils, such as olive oil, and injectable organic esters, such as ethyl oleate. The proper fluidity may be maintained, for example, by the use of a coating such as lecithin, by the maintenance of the required particle size in the case of dispersion and by the use of surfactants. In many cases, it will be preferable to include isotonic agents, for example, sugars, polyalcohols such as glycerol, mannitol, sorbitol, or sodium chloride in the composition. Prolonged absorption of the injectable compositions may be brought about by including in the composition an agent that delays absorption, for example, monostearate salts and gelatin. Sterile injectable solutions may be prepared by incorporating the active compound in the required amount in an appropriate solvent with one or a combination of ingredients e.g. as enumerated above, as required, followed by sterilization microfiltration. Generally, dispersions are prepared by incorporating the active compound into a sterile vehicle that contains a basic dispersion medium and the required other ingredients e.g. from those enumerated above. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

Sterile injectable solutions may be prepared by incorporating the active compounds in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, examples of methods of preparation are vacuum-drying and freeze-drying (lyophilization) that yield a powder of the active ingredient plus any additional desired ingredient from a previously sterile-filtered solution thereof.

In some aspects of the disclosure, a kit-of-parts is provided comprising:
a. a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction; and
b. an antibody-drug conjugate comprising an antibody or fragment thereof that specifically binds to a target expressed on the surface of a tumour cell.

In some aspects of the disclosure, a kit-of-parts is provided comprising:
a. a bispecific antibody that comprises:
(a) an antibody or fragment thereof that specifically binds to PD-L1, and
(b) an antibody or fragment thereof that specifically binds to VEGF; and
b. an antibody-drug conjugate comprising an antibody or fragment thereof that specifically binds to a target expressed on the surface of a tumour cell.

The embodiments for the combination therapy disclosed herein comprising the binding agent (e.g. the bispecific antibody) in combination with the antibody-drug conjugate can be used in the kit-of-parts disclosed herein. Features described herein in more detail among others for the “binding agent” embodiments (e.g. the “bispecific antibody” embodiments), the “antibody-drug conjugate components” embodiments, the “linker and payload moieties” embodiments, the “linker-payload” embodiments, and the “antibody-drug conjugate composition” embodiments equally apply to the kit-of-parts embodiments disclosed herein which may include the binding agent (e.g. the bispecific antibody) and antibody-drug conjugates disclosed herein.

In some embodiments, the binding agent (e.g. the bispecific antibody) and the antibody-drug conjugate are comprised in separate containers. In some embodiments, the kit-of-parts further comprises instructions for use.

As used herein, an “instructional material” or “instructions” includes a publication, a recording, a diagram, or any other medium of expression which can be used to communicate the usefulness of the compositions and methods described herein. The instructional material of the kit of the disclosure may, for example, be affixed to a container which contains the compositions of the disclosure or be shipped together with a container which contains the compositions. Alternatively, the instructional material may be shipped separately from the container with the intention that the instructional material and the compositions be used cooperatively by the recipient.

In some embodiments, the binding agent (e.g. the bispecific antibody) and the antibody-drug conjugate are for diagnostic use, preferably for diagnosing a cancer as described herein.

The embodiments for the combination therapy disclosed herein comprising the binding agent (e.g. bispecific antibody) in combination with the antibody-drug conjugate can be used in the kit-of-parts disclosed herein. Features described herein in more detail among others for the “binding agent” embodiments (e.g. the “bispecific antibody” embodiments), the “antibody-drug conjugate components” embodiments and the “antibody-drug conjugate” embodiments equally apply to the kit-of-parts disclosed herein.

The present invention further provides the combinations, medical preparations, compositions and kits described herein for use as a medicament. The medicament may be for administration to a subject. The use as a medicament may comprise a therapeutic or prophylactic treatment of a disease or disorder, for example comprising treating or preventing cancer. In some embodiments, the therapeutic or prophylactic treatment of a disease or disorder further comprises administering radiotherapy, preferably local radiotherapy.

### Additional Therapeutic Agents/Treatments

Combination strategies in cancer treatment may be desirable due to a resulting synergistic effect, which may be considerably stronger than the impact of an approach using only one anti-cancer agent.

Thus, in certain embodiments of aspects of the present disclosure, the combination, medical preparation or composition may further comprise one or more further therapeutic agents other than the ADC and the anti-PD-1/PD-L1xVEGF/VEGFR binding agent described herein.

In certain embodiments, the combination, medical preparation or composition of the aspects of the present disclosure may be accompanied by additional treatments, e.g., one or more selected from radiation therapy, surgery, and hyperthermia therapy. In some embodiments, the additional treatment is radiotherapy, preferably local radiotherapy.

In certain embodiments of the aspects of the present disclosure, the combination, medical preparation or composition further comprises one or more further therapeutic agents other than the ADC described herein and other than the anti-PD-1/PD-L1xVEGF/VEGFR binding agent described herein and is accompanied by one or more additional treatments.

In some embodiments, the further therapeutic agent is selected from an immune checkpoint inhibitor, a chemotherapeutic agent, or a combination thereof. In some embodiments, the further therapeutic agent is an immune checkpoint inhibitor. In some embodiments, the further therapeutic agent is a chemotherapeutic agent.

The description (including the following examples) is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### EXAMPLES

### Example 1: Study to Assess the Therapeutic Activity of an Anti-hHER2-ADC (Compound 1) in Combination with an Anti-mPDL1xmVEGF in a Syngeneic Mouse Model

To investigate the combination of the anti-hHER2-ADC (Compound 1) and anti-mPDL1xmVEGF (mouse surrogate of Compound 5, in which the anti-VEGF part is changed to an anti-mouse-VEGF part and the anti-PDL1 VHH domain is exchanged to a scFv of atezolizumab, which is mouse cross-reactive)), a syngeneic mouse model was established as follows: C57BL/6 mice were subcutaneously transplanted with 1×10⁶ hHER2-hPDL1-MC38 cells. Treatments with the ADC/antibody indicated below were started when the tumour size was approximately 100 mm³. One day before treatment start 0.5 mg of an anti-mouse CD20 antibody were administered to deplete the B cells. 20 mg/kg of the anti-hHER2-ADC were administered intravenously (i.v.) twice, once weekly (QW*2). 5 mg/kg or 20 mg/kg of the anti-mPDL1xmVEGF were administered intraperitoneally (i.p.) 4 times every second day (Q2D*4). Monotherapy control groups G2, G3 and G4 were treated only either with the anti-mPDL1xmVEGF antibody or the anti-hHER2 ADC Compound 1. The Vehicle control group G1 was treated with 0.9% sodium chloride injection only. Tumour volume and body weight were monitored. The study design is summarized in Table I.

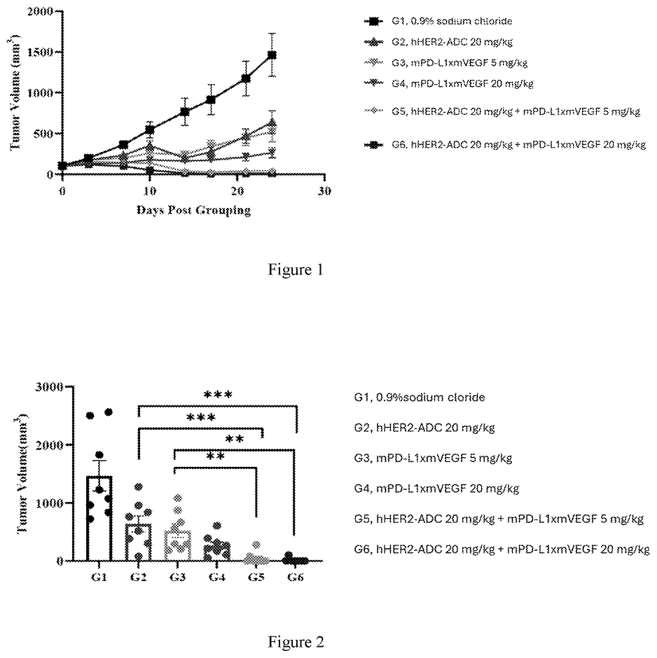

The combination of the anti-hHER2-ADC and the anti-mPDL1xmVEGF resulted in tumour regression and long-term tumour control, whereas monotherapy treatments resulted in shorter-term tumour control and outgrowth of tumours (FIG. 1). Tumour volumes at day of study determination in the combination therapy group were significantly lower than compared with the two monotherapy groups (FIG. 2).

TABLE I
Study design anti-hHER2-ADC (Compound
1) + anti-mPDL1xmVEGF:

| Groups | Treatment | Dose | Dosing Schedule |
|---|---|---|---|
| G1 | Vehicle (0.9% | — | QW*2 and Q2D*4 |
| | sodium chloride) | | |
| G2 | Compound 1 | 20 mg/kg | QW*2 |
| G3 | anti-mPDL1xmVEGF | 5 mg/kg | Q2D*4 |
| G4 | anti-mPDL1xmVEGF | 20 mg/kg | Q2D*4 |
| G5 | Compound 1 + | 20 mg/kg and | QW*2 and Q2D*4 |
| | anti-mPDL1xmVEGF | 5 mg/kg | |
| G6 | Compound 1 + | 20 mg/kg and | QW*2 and Q2D*4 |
| | anti-mPDL1xmVEGF | 20 mg/kg | |

### Example 2: Study to Assess the Therapeutic Activity of an Anti-hHER2-ADC (Compound 1) in Combination with an Anti-hPDL1xhVEGF (Compound 5) in a Human Xenograft PBMC Mouse Model

To investigate the combination of the anti-hHER2-ADC (Compound 1) and the anti-hPDL1xhVEGF (Compound 5), a A375 melanoma cancer xenograft mouse model engrafted with human PBMCs was established as follows: B-NDG B2M KO plus (vendor Biocytogen) mice were subcutaneously transplanted with A375 cells overexpressing hHER2 and co-injected with human PBMCs. The human A375 cells endogenously express hPDL1 and hHER2 but were additionally transduced with human HER2. Treatments with the ADC/antibody indicated below were started when the tumour size was approximately 300 mm³. 20 mg/kg of anti-hPDL1xhVEGF (Compound 5) and 2 mg/kg of the hHER2-ADC (Compound 1) were administered 4 times every second day (Q2D*4). Monotherapy control groups G2 andG3 were treated only either with the anti-hPDL1xhVEGF (Compound 5) or the anti-hHER2 ADC (Compound 1). The Vehicle control group G1 was treated with PBS only. Tumour volume and body weight were monitored. The study design is summarized in Table II.

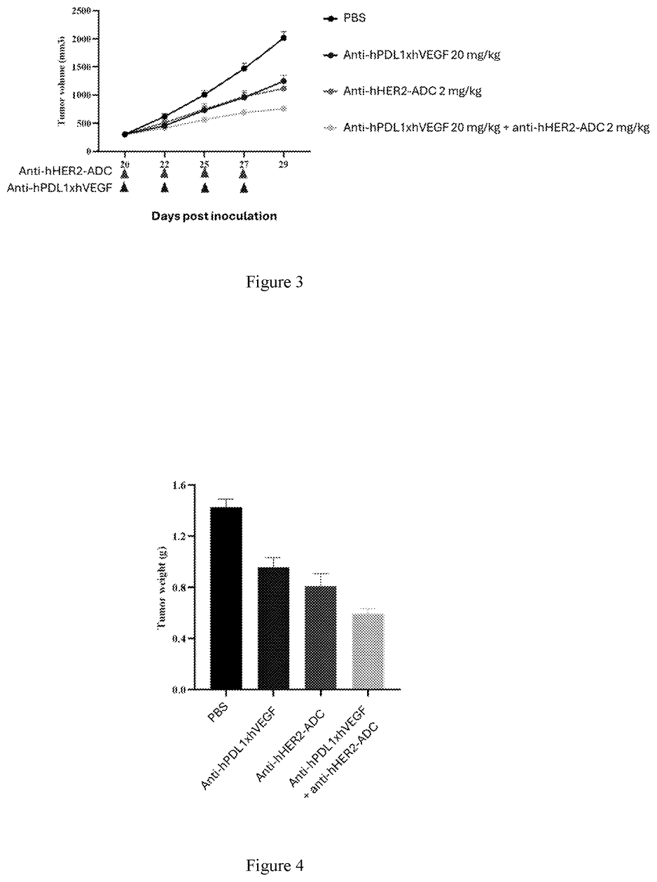

The combination of the anti-hHER2 ADC and the anti-hPDL1xhVEGF showed a higher tumour growth inhibition compared with each monotherapy treatment (FIG. 3). Tumour weights after study determination in the combination therapy group were lower than compared with the two monotherapy groups (FIG. 4).

TABLE II
Study design anti-hHER2-ADC (Compound
1) + anti-hPDL1xhVEGF (Compound 5)

| Groups | Treatment | Dose | Dosing Schedule |
|---|---|---|---|
| G1 | Vehicle (PBS) | — | Q2D*4 |
| G2 | Compound 5 | 20 mg/kg | Q2D*4 |
| G3 | Compound 1 | 2 mg/kg | Q2D*4 |
| G4 | Compound 5 + | 20 mg/kg and | Q2D*4 |
| | Compound 1 | 2 mg/kg | |

### Example 3: Study to Assess the Therapeutic Activity of an Anti-hB7-H3-ADC (Compound 2) in Combination with an Anti-mPDL1xmVEGF in a Syngeneic Mouse Model

To investigate the combination of the anti-hB7-H3-ADC (Compound 2) and anti-mPDL1xmVEGF (mouse surrogate of Compound 5, in which the anti-VEGF part is changed to an anti-mouse-VEGF part and the anti-PDL1 VHH domain is exchanged to a scFv of atezolizumab, which is mouse cross-reactive), a syngeneic mouse model was established as follows: BALB/c mice were subcutaneously transplanted with 6×10⁵ hB7-H3-CT26 cells.

Treatments with the ADC/antibody indicated below were started when the tumour size was approximately 100 mm³. 10 mg/kg of the anti-hB7-H3-ADC were administered intravenously (i.v.) once every 2 days for 5 consecutive doses (Q2D*5). 20 mg/kg of the anti-mPDL1xmVEGF were administered intraperitoneally (i.p.) once every 7 days for 2 consecutive doses (Q7D*2). Monotherapy control groups G2 and G3 were treated only either with the anti-mPDL1xmVEGF antibody or the anti-hB7-H3 ADC Compound 2. The Vehicle control group G1 was treated with PBS only. Tumour volume and body weight were monitored. The study design is summarized in Table III.

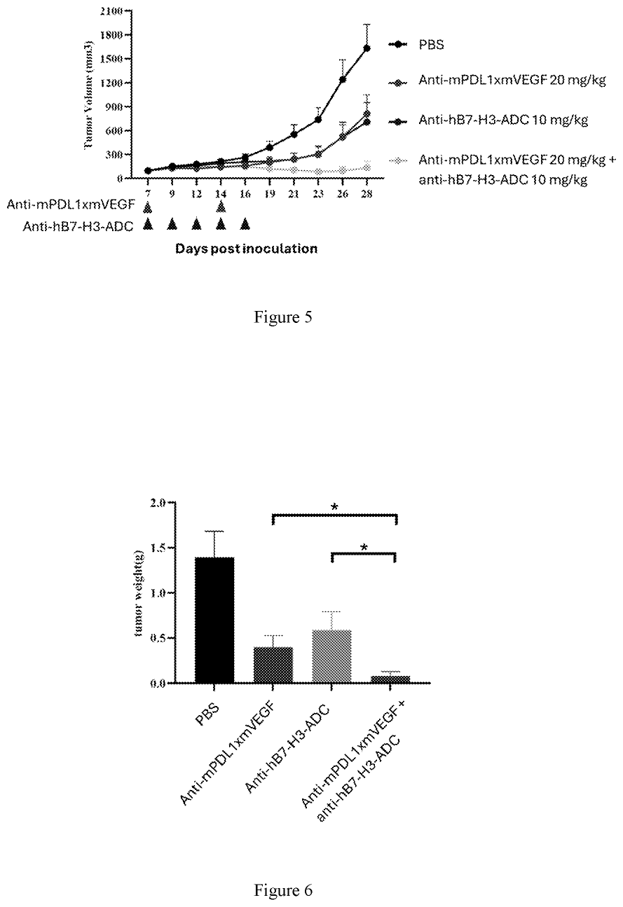

The combination of the anti-hB7-H3-ADC and the anti-mPDL1xmVEGF resulted in tumour regression and long-term tumour control, whereas monotherapy treatments resulted in shorter-term tumour control and outgrowth of tumours (FIG. 5). Tumour weights after study determination in the combination therapy group were significantly lower than compared with the two monotherapy groups (FIG. 6).

TABLE III
Study design anti-hB7-H3-ADC (Compound
2) + anti-mPDL1xmVEGF:

| Groups | Treatment | Dose | Dosing Schedule |
|---|---|---|---|
| G1 | Vehicle (PBS) | — | Q2D*5 |
| G2 | anti-mPDL1xmVEGF | 20 mg/kg | Q7D*2 |
| G3 | Compound 2 | 10 mg/kg | Q2D*5 |
| G4 | Compound 2 + | 10 mg/kg and | Q2D*5and Q7D*2 |
| | anti-mPDL1xmVEGF | 20 mg/kg | |

### Example 4: Study to Assess the Therapeutic Activity of an Anti-hB7-H3-ADC (Compound 2) in Combination with an Anti-hPDL1xhVEGF (Compound 5) in a Human Xenograft PBMC Mouse Model

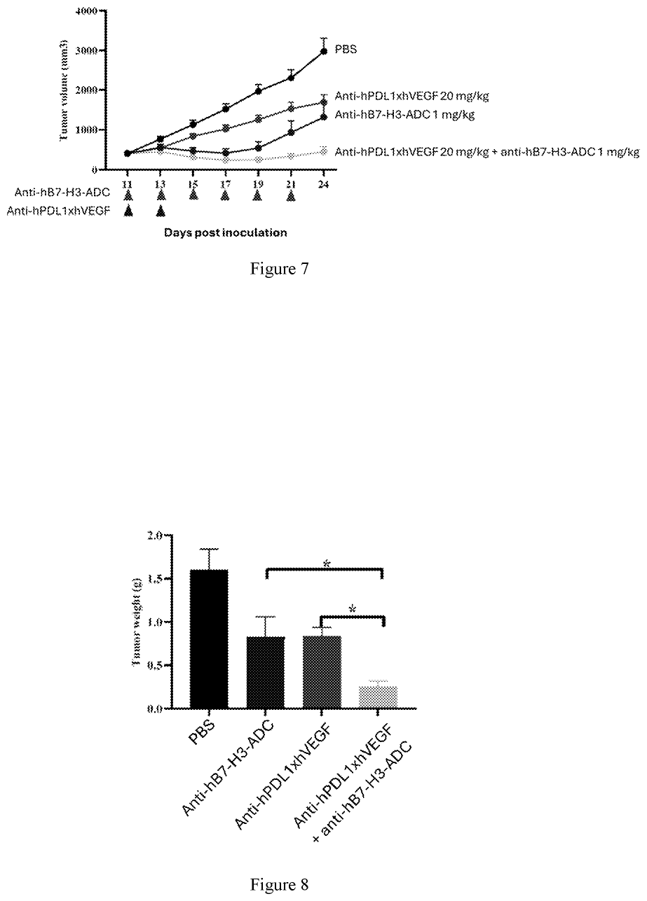

To investigate the combination of the anti-hB7-H3-ADC (Compound 2) and anti-hPDL1xhVEGF (Compound 5) a A375 melanoma cancer xenograft mouse model engrafted with human PBMCs was established as follows: B-NDG B2M KO plus (vendor Biocytogen) mice were subcutaneously transplanted with A375 cells and co-injected with human PBMCs. The human A375 cells endogenously express human B7-H3 and human PD-L1. Treatments with the ADC/antibody indicated below were started when the tumour size was approximately 300 mm³. 20 mg/kg of anti-hPDL1xhVEGF (Compound 5) were administered 6 times every second day (Q2D*6). 1 mg/kg of the anti-hB7H3-ADC (Compound 2) was administered twice. Monotherapy control groups G2, and G3 were treated only either with the anti-hPDL1xhVEGF (Compound 5) or the anti-hB7-H3 ADC compound (Compound 2). The Vehicle control group G1 was treated with PBS only. Tumour volume and body weight were monitored for fourteen days after treatment start (FIG. 7). Then the study was terminated, and tumours were taken out and tumour weights were measured (FIG. 8). The study design is summarized in Table IV.

The combination of the anti-hB7-H3 ADC and the anti-hPDL1xhVEGF resulted in tumour regression and tumour control, whereas monotherapy treatments resulted in shorter-term tumour control and outgrowth of tumours (FIG. 7). Tumour weights after study determination in the combination therapy group were significantly lower than compared with the two monotherapy groups (FIG. 8).

TABLE IV
Study design anti-hB7-H3-ADC (Compound
2) + anti-hPDL1xhVEGF (Compound 5):

| Groups | Treatment | Dose | Dosing Schedule |
|---|---|---|---|
| G1 | Vehicle (PBS) | — | Q2D*6 |
| G2 | Compound 5 | 20 mg/kg | Q2D*6 |
| G3 | Compound 2 | 1 mg/kg | Q2D*2 |
| G4 | Compound 5 + | 20 mg/kg and | Q2D*6 and Q2D*2 |
| | Compound 2 | 1 mg/kg | |

### Example 5: Study to Assess the Therapeutic Activity of an Anti-hTROP2-ADC (Compound 3) in Combination with Anti-mPDL1xmVEGF in a Syngeneic Mouse Model

To investigate the combination of the anti-hTROP2-ADC (Compound 3) and anti-mPDL1xmVEGF (mouse surrogate of Compound 5, in which the anti-VEGF part is changed to an anti-mouse-VEGF part and the anti-PDL1 VHH domain is exchanged to a scFv of atezolizumab, which is mouse cross-reactive), a syngeneic mouse model was established as follows: C57BL/6 mice were subcutaneously transplanted with hTROP2-MC38 cells. Treatments with the ADC/antibody indicated below were started when the tumour size was approximately 100 mm³. One day before treatment start 0.5 mg of an anti-mouse CD20 antibody were administered to deplete the B cells, in order to slow down anti-drug antibody development. 20 mg/kg of anti-hTROP2-ADC (Compound 3) were administered intravenously (i.v.) 4 times, once weekly (QW*4). 5 mg/kg or 20 mg/kg of the anti-mPDL1xmVEGF were administered intraperitoneally (i.p.) 4 times every second day (Q2D*4). Monotherapy control groups G2 to G4 were treated only either with the anti-mPDL1xmVEGF or the anti-hTROP2 ADC (Compound 3). The Vehicle control group G1 was treated with 0.9% sodium chloride only. Tumour volume and body weight were monitored. The study design is summarized in Table V.

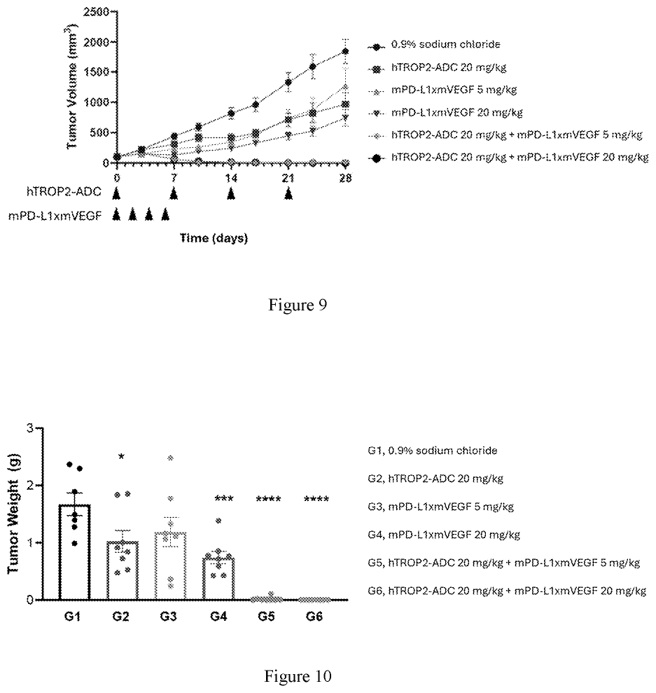

The combination of the anti-hTROP2-ADC and the anti-mPDL1xmVEGF resulted in complete tumour regression, whereas monotherapy treatments resulted only in short-term tumour control and outgrowth of tumours (FIG. 9). Tumour weights after study determination in the combination therapy group were lower than compared withthe two monotherapy groups (FIG. 10).

Tumor growth inhibition (TGI) was calculated according to the formula TGI=(Vc−Vt)/(Vc−Vo)×100%, where Vc is the average tumor volume of the negative control group at the end of the study, Vt is the average tumor volume of the treatment group at the end of the study, and Vo is the average tumor volume of the negative control group at the time of grouping and administration. In the syngeneic hTROP2+MC38 model, Compound 3 combined with an anti-mPD-L1xmVEGF showed tumor growth inhibition of 106.1% and 106.9% for 2 dose levels (DL) of the anti-mPD-L1xmVEGF vs. 51.4% for Compound 3 alone or 47.6% and 71.4% for the anti-mPD-L1xmVEGF at DL1 and DL2, respectively.

TABLE V
Study design anti-hTROP2-ADC (Compound
3) + anti-mPDL1xmVEGF:

| Groups | Treatment | Dose | Dosing Schedule |
|---|---|---|---|
| G1 | Vehicle (0.9% | — | QW*4 and Q2D*4 |
| | sodium chloride) | | |
| G2 | Compound 3 | 20 mg/kg | QW*4 |
| G3 | anti-mPDL1xmVEGF | 5 mg/kg | Q2D*4 |
| G4 | anti-mPDL1xmVEGF | 20 mg/kg | Q2D*4 |
| G5 | Compound 3 + | 20 mg/kg and | QW*4 and Q2D*4 |
| | anti-mPDL1xmVEGF | 5 mg/kg | |
| G6 | Compound 3 + | 20 mg/kg and | QW*4 and Q2D*4 |
| | anti-mPDL1xmVEGF | 20 mg/kg | |

### Example 6: Study to Assess the Therapeutic Activity of an Anti-hTROP2-ADC (Compound 3) in Combination with an Anti-hPDL1xhVEGF (Compound 5) in a Human Xenograft PBMC Mouse Model

To investigate the combination of the anti-hTROP2-ADC (Compound 3) and the anti-hPDL1xhVEGF (Compound 5), a A375 melanoma cancer xenograft mouse model engrafted with human PBMCs was established as follows: B-NDG B2M KO plus (vendor Biocytogen) mice were subcutaneously transplanted with A375 cells overexpressing hTROP2 and co-injected with human PBMCs. The human A375 cells endogenously express hPDL1 and marginally hTROP2. Therefore, the cells were additionally transduced with human TROP2. Treatments with the ADC/antibody indicated below were started when the tumour size was approximately 300 mm³. 20 mg/kg of anti-hPDL1xhVEGF were administered 2 times (once every two days, Q2D*2). 1 mg/kg of the anti-hTROP2-ADC (Compound 3) was administered times (once every two days, Q2D*5). Monotherapy control groups G2 and G3 were treated only either with the anti-hPDL1xhVEGF (Compound 5) or the anti-hTROP2 ADC (Compound 3). The Vehicle control group G1 was treated with PBS only. Tumour volume and body weight were monitored. The study design is summarized in Table VI.

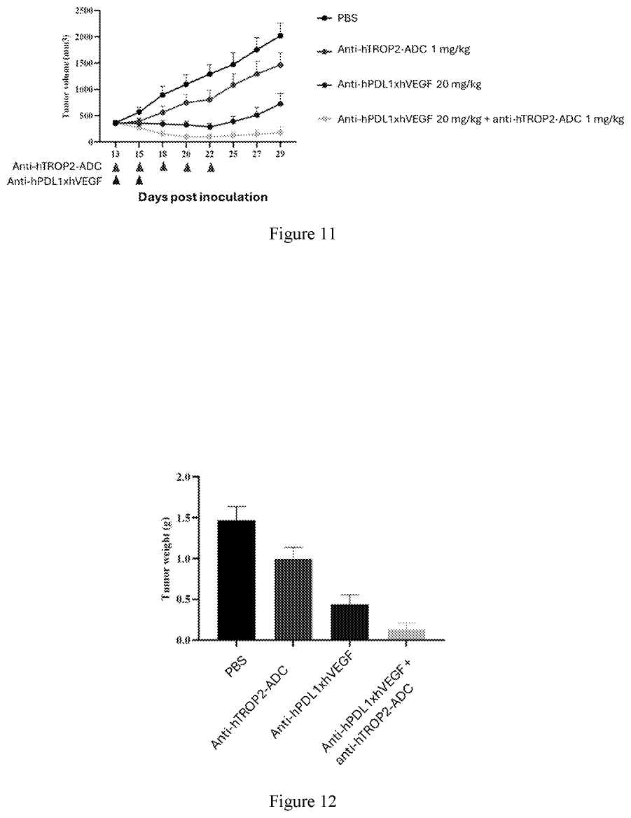

Similarly to the result obtained with Compound 3 and the mouse surrogate of Compound 5 in the previous example, the combination of the anti-hTROP2 ADC Compound 3 and the anti-hPDL1xhVEGF Compound 5 resulted in tumour regression and tumour control, whereas monotherapy treatments resulted at best in shorter-term tumour control followed by outgrowth of tumours (FIG. 11). Tumour weights after study determination in the combination therapy group were lower than compared with the two monotherapy groups (FIG. 12).

Tumor growth inhibition (TGI) was calculated according to the formula TGI=(Vc−Vt)/(Vc−Vo)×100%, where Vc is the average tumor volume of the negative control group at the end of the study, Vt is the average tumor volume of the treatment group at the end of the study, and Vo is the average tumor volume of the negative control group at the time of grouping and administration. In the xenograft A375+PBMC co-injection model, the combination of Compound 3 and Compound 5 showed tumor growth inhibition of 111.3% vs. 33.6% for Compound 3 or 78.2% for Compound 5 alone.

TABLE VI
Study design anti-hTROP2-ADC (Compound
3) + anti-hPDL1xhVEGF (Compound 5):

| Groups | Treatment | Dose | Dosing Schedule |
|---|---|---|---|
| G1 | Vehicle | — | Q2D*5 |
| G2 | Compound 5 | 20 mg/kg | Q2D*2 |
| G3 | Compound 3 | 1 mg/kg | Q2D*5 |
| G4 | Compound 5 + | 20 mg/kg and | Q2D*2 and Q2D*5 |
| | Compound 3 | 1 mg/kg | |

### Example 7: Study to Assess the Therapeutic Activity of an Anti-hHER3-ADC (Compound 4) in Combination with an Anti-Mouse mPDL1xmVEGF Bispecific Antibody in a Syngeneic Mouse Model

To investigate the combination of anti-hHER3-ADC (Compound 4) and anti-mPDL1xmVEGF (mouse surrogate of Compound 5, in which the anti-VEGF part is changed to an anti-mouse-VEGF part and the anti-PDL1 VHH domain is exchanged to a scFv of atezolizumab, which is mouse cross-reactive), a syngeneic mouse model is established as follows: C57BL/6J mice were subcutaneously inoculated with 1×10⁶ MC38 murine colon adenocarcinoma cells transgenic for human Her3 (hHER3-MC38). Treatments with the ADC/antibody indicated below were started when the tumour size was approximately 60 mm³. 10 mg/kg of the anti-hHER3-ADC are administered intravenously (i.v.) 3 times on day 0, 3 and 7. 10 mg/kg of the anti-mPDL1xmVEGF were administered intraperitoneally (i.p.) 3 times on day 0, 3 and 7. Monotherapy control groups G2 and G3 were treated only either with the anti-hHER3-ADC or the anti-mPDL1xmVEGF compound. The Vehicle control group G1 was treated with saline only. Tumour volume and body weight were monitored. The study design is summarized in Table VII.

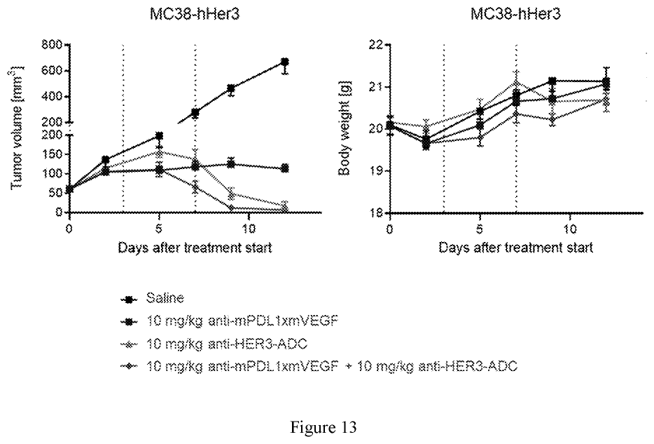

Anti-HER3-ADC (Compound 4) combined with the anti-mPDL1xmVEGF showed a general trend towards enhanced anti-tumour efficacy compared to the respective monotherapy treated groups in-between Day 7 and the end of study on Day 14 (FIG. 13). The tumour volumes in the combination group were significantly lower than in the corresponding monotherapy groups on Day 9 (Anti-HER3-ADC+anti-mPDL1xmVEGF 12.41 mm³; Anti-HER3-ADC 49.33 mm³, p=0.0133; anti-mPDL1xmVEGF 125.32 mm³, p=4.75E-07). Body weight measurements did not show relevant drops in body weight.

TABLE VII
Study design anti-hHER3-ADC (Compound
4) + anti-mPDL1xmVEGF:

| | | Dose | Dosing | Number of |
|---|---|---|---|---|
| Group | Treatment | (mg/kg) | Schedule | doses |
| G1 | Vehicle | — | D 0, D 3 and D 7 | 3 |
| G2 | Compound 4 | 10 | D 0, D 3 and D 7 | 3 |
| G3 | anti-mPDL1xmVEGF | 10 | D 0, D 3 and D 7 | 3 |
| G4 | Compound 4 + | 10 | D 0, D 3 and D 7 | 3 |
| | anti-mPDL1xmVEGF | | | |

Note:
BIW means twice a week dosing.

### Example 8: Study to Assess the Therapeutic Activity of an Anti-hHER3-ADC (Compound 4) in Combination with an Anti-hPDL1xhVEGF (Compound 5) in a Human Xenograft PBMC Mouse Model

To investigate the combination of anti-hHER3-ADC (Compound 4) and the anti-hPDL1xhVEGF (Compound 5, with humanized anti-PD-L1 single heavy-chain variable (VHH) domains fused to an anti-VEGF-A IgG1 antibody (Bevacizumab) containing Fc-silencing mutations), a xenograft mouse model engrafted with human PBMCs was established as follows: B-NDG B2M KO plus (vendor Biocytogen) mice were subcutaneously inoculated with 4.5×10⁶ A375 (melanoma) and on the same day co-injected with 10*10⁶ human PBMCs intraperitoneally (i.p.). The human A375 cells endogenously express hPDL1 and hHER3. Treatments with the ADC/antibody indicated below were started when the tumour size was approximately 100 mm³. 20 mg/kg of Compound 5 were administered 3 times on day 0, 3 and 7. 1 mg/kg of Compound 4 were administered one time (DO). Monotherapy control groups G2 and G3 were treated only either with the anti-hPDL1xhVEGF Compound 5 or the anti-hHER3 ADC Compound 4. The Vehicle control group G1 was treated with PBS only. Tumour volume and body weight were monitored. The study design is summarized in Table VIII.

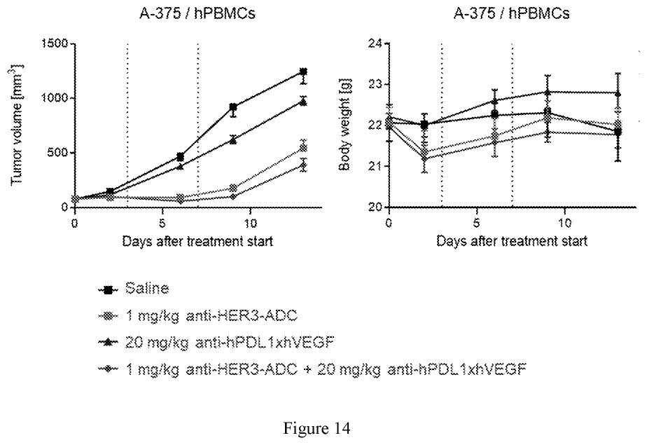

Anti-hHER3 ADC (Compound 4) combined with anti-hPDL1xhVEGF (Compound 5) showed a general trend towards enhanced anti-tumour efficacy compared to the respective monotherapy treated groups (FIG. 14). Body weight measurements did not show relevant drops in body weight.

TABLE VIII
Study design anti-hHER3-ADC (Compound
4) + anti-hPDL1xhVEGF (Compound 5):

| Groups | Treatment | Dose | Dosing Schedule |
|---|---|---|---|
| G1 | Vehicle | — | |
| G2 | Compound 5 | 20 mg/kg | D 0, D 3 and D 7 |
| G3 | Compound 4 | 1 mg/kg | D 0 |
| G4 | Compound 5 + | 20 mg/kg and | D 0, D 3 and |
| | Compound 4 | 1 mg/kg | D 7and D 0 |

### Example 9: Study to Assess the Therapeutic Activity of an Anti-hHER3-ADC (Compound 4) in Combination with an Anti-Mouse mPDL1xmVEGF Bispecific Antibody in a Syngeneic Mouse Model with an Optimized Dose of Anti-HER3-ADC

To investigate the combination of anti-hHER3-ADC (Compound 4) and anti-mPDL1xmVEGF (mouse surrogate of Compound 5, as also used in Example 7) a follow-up study of the syngeneic mouse model as described in example 7 was conducted as follows: C57BL/6J mice were subcutaneously inoculated with 1×10⁶ hHER3-MC38 cells. Treatments with the ADC/antibody indicated below were started when the tumour size was approximately 60 mm³. Compared to the study described in example 7, in this study the dose of the anti-hHER3-ADC was reduced to 3 mg/kg; anti-mPDL1xmVEGF was administered at 10 mg/kg, which was the same as in example 7. 3 mg/kg of anti-hHER3-ADC was administered intravenously (i.v.) 3 times on day 0, 3 and 7. 10 mg/kg of the anti-mPDL1xmVEGF were administered intraperitoneally (i.p.) 3 times on day 0, 3 and 7. Monotherapy control groups G2 and G3 were treated only either with the anti-hHER3-ADC or the anti-mPDL1xmVEGF compound. The Vehicle control group G1 was treated with saline only. Tumour volume and body weight were monitored. The study design is summarized in Table IX.

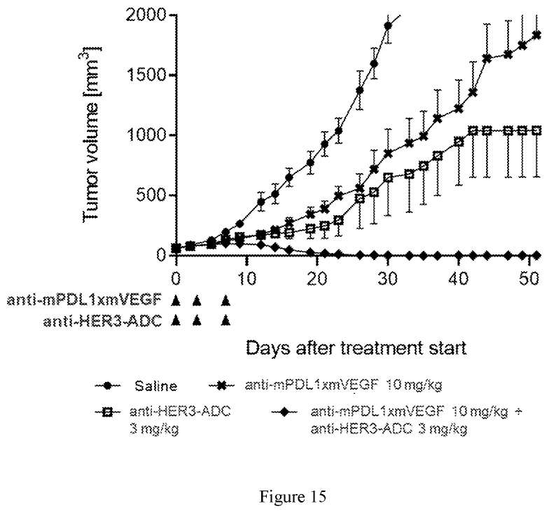

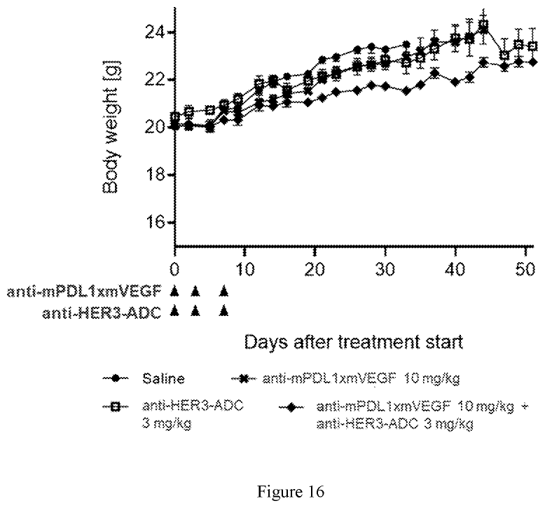

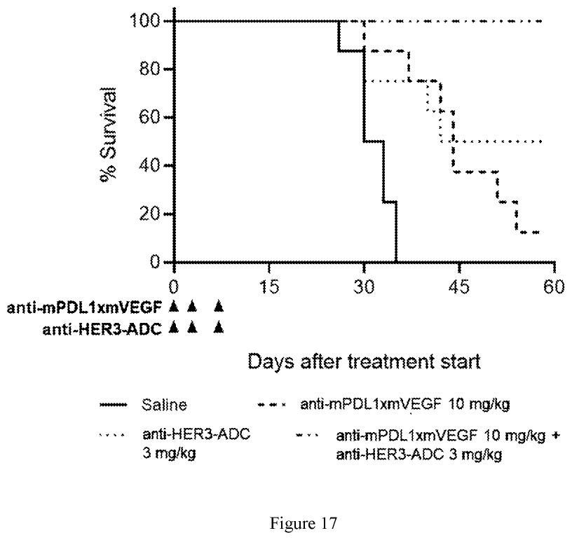

Anti-HER3-ADC (Compound 4) combined with the anti-mPDL1xmVEGF showed very strong anti-tumour efficacy and resulted in complete tumour regression whereas the respective monotherapy treated groups showed weaker tumour growth inhibition with delayed tumour growth (FIG. 15). Body weight measurements did not show any drops in body weight (FIG. 16). Survival analysis revealed a clearly prolonged survival of the anti-HER3-ADC+anti-mPDL1xmVEGF combination group compared to the respective monotherapy groups (FIG. 17).

TABLE IX
Study design anti-hHER3-ADC (Compound
4) + anti-mPDL1xmVEGF:

| | | Dose | Dosing | Number of |
|---|---|---|---|---|
| Group | Treatment | (mg/kg) | Schedule | doses |
| G1 | Vehicle | — | D 0, D 3 | 3 |
| | | | and D 7 | |
| G2 | Compound 4 | 3 | D 0, D 3 | 3 |
| | | | and D 7 | |
| G3 | anti-mPDL1xmVEGF | 10 | D 0, D 3 | 3 |
| | | | and D 7 | |
| G4 | Compound 4 + | 3 and 10 | D 0, D 3 | 3 |
| | anti-mPDL1xmVEGF | | and D 7 | |

### Example 10: Study to Assess the Therapeutic Activity of an Anti-hHER2-ADC (Compound 1) in Combination with an Anti-hPD1xhVEGF (Compound 13) in a Human Xenograft PBMC Mouse Model

To investigate the combination of the anti-hHER2-ADC (Compound 1) and the anti-hPD1xhVEGF (Compound 13), a RKO xenograft mouse model engrafted with human PBMCs was established as follows: B-NDG B2M KO plus (vendor Biocytogen) mice were subcutaneously transplanted with 2*10⁶ RKO cells and co-injected with 1*10⁶ human PBMCs. Treatments with the ADC/antibody indicated below were started when the tumour size was approximately 100 mm³. 10.7 mg/kg of anti-hPD1xhVEGF (Compound 13) was injected once at the left side of the mouse flank and 2 mg/kg of the hHER2-ADC (Compound 1) was injected once at the right side of the mouse flank. Monotherapy control groups G2 and G3 were treated only either with the anti-hPD1xhVEGF (Compound 13) or the anti-hHER2 ADC (Compound 1). The Vehicle control group G1 was treated with PBS only. Tumour volume and body weight were monitored. The study design is summarized in Table X.

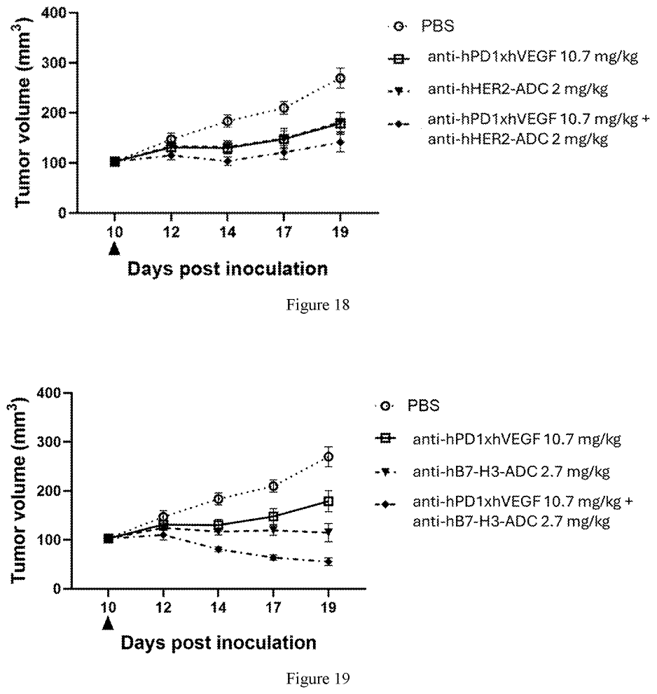

The combination of the anti-hHER2 ADC and the anti-hPD1xhVEGF showed a higher tumour growth inhibition compared with each monotherapy treatment (FIG. 18).

TABLE X
Study design anti-hHER2-ADC (Compound
1) + anti-hPD1xhVEGF (Compound 13)

| Groups | Treatment | Dose | Dosing Schedule |
|---|---|---|---|
| G1 | Vehicle (PBS) | — | Once |
| G2 | Compound 13 | 10.7 mg/kg | Once |
| G3 | Compound 1 | 2 mg/kg | Once |
| G4 | Compound 13 + | 10.7 mg/kg and | Once |
| | Compound 1 | 2 mg/kg | |

### Example 11: Study to Assess the Therapeutic Activity of an Anti-hB7-H3-ADC (Compound 2) in Combination with an Anti-hPD1xhVEGF (Compound 13) in a Human Xenograft PBMC Mouse Model

To investigate the combination of the anti-hB7-H3-ADC (Compound 2) and anti-hPD1xhVEGF (Compound 13) a RKO xenograft mouse model engrafted with human PBMCs was established as follows: B-NDG B2M KO plus (vendor Biocytogen) mice were subcutaneously transplanted with 2.5*10⁶ RKO cells and co-injected with 1*10⁶ human PBMCs. Treatments with the ADC/antibody indicated below were started when the tumour size was approximately 100 mm³. 10.7 mg/kg of anti-hPD1xhVEGF (Compound 13) was injected once at the left side of the mouse flank. 2.7 mg/kg of the anti-hB7H3-ADC (Compound 2) was administered once at the right side of the mouse flank. Monotherapy control groups G2, and G3 were treated only either with the anti-hPD1xhVEGF (Compound 13) or the anti-hB7-H3 ADC compound (Compound 2). The Vehicle control group G1 was treated with PBS only. Tumour volume and body weight were monitored for about fourteen days after treatment start. The study design is summarized in Table XI.

The combination of the anti-hB7-H3 ADC and the anti-hPD1xhVEGF resulted in tumour regression and a higher tumour growth inhibition compared with each monotherapy treatment (FIG. 19).

TABLE XI
Study design anti-hB7-H3-ADC (Compound
2) + anti-hPD1xhVEGF (Compound 5):

| Groups | Treatment | Dose | Dosing Schedule |
|---|---|---|---|
| G1 | Vehicle (PBS) | — | Once |
| G2 | Compound 13 | 10.7 mg/kg | Once |
| G3 | Compound 2 | 2.7 mg/kg | Once |
| G4 | Compound 13 + | 10.7 mg/kg and | Once |
| | Compound 2 | 2.7 mg/kg | |

### Example 12: Study Design to Assess the Therapeutic Activity of an Anti-hTROP2-ADC (Compound 3) in Combination with an Anti-hPD1xhVEGF (Compound 13) in a Human Xenograft PBMC Mouse Model

To investigate the combination of the anti-hTROP2-ADC (Compound 3) and the anti-hPD1xhVEGF (Compound 13), a A375 melanoma cancer xenograft mouse model engrafted with human PBMCs is established as follows: B-NDG B2M KO plus (vendor Biocytogen) mice are subcutaneously transplanted with A375 cells overexpressing hTROP2 and co-injected with human PBMCs. The human A375 cells endogenously express hPDLT and marginally hTROP2. Therefore, the cells are additionally transduced with human TROP2. Treatments with the ADC/antibody indicated below are started when the tumour size is approximately 300 mm³. 20 mg/kg of anti-hPD1xhVEGF are administered 2 times (once every two days, Q2D*2). 1 mg/kg of the anti-hTROP2-ADC (Compound 3) is administered 5 times (once every two days, Q2D*5). Monotherapy control groups G2 and G3 are treated only either with the anti-hPD1xhVEGF (Compound 13) or the anti-hTROP2 ADC (Compound 3). The Vehicle control group G1 is treated with PBS only. Tumour volume and body weight are monitored. The study design is summarized in Table XII.

The combination of the anti-hTROP2 ADC Compound 3 and the anti-hPD1xhVEGF Compound 13 is expected to result in tumour regression and tumour control, whereas monotherapy treatments result only in shorter-term tumour control.

TABLE XII
Study design anti-hTROP2-ADC (Compound
3) + anti-hPD1xhVEGF (Compound 13):

| Groups | Treatment | Dose | Dosing Schedule |
|---|---|---|---|
| G1 | Vehicle | — | Q2D*5 |
| G2 | Compound 13 | 20 mg/kg | Q2D*2 |
| G3 | Compound 3 | 1 mg/kg | Q2D*5 |
| G4 | Compound 13 + | 20 mg/kg and | Q2D*2 and Q2D*5 |
| | Compound 3 | 1 mg/kg | |

### Example 13: Study Design to Assess the Therapeutic Activity of an Anti-HER3-ADC (Compound 4) in Combination with an Anti-hPD1xhVEGF (Compound 13) in a Human Xenograft PBMC Mouse Model

To investigate the combination of the anti-HER3-ADC (Compound 4) and anti-hPD1xhVEGF (Compound 13) a A375 melanoma cancer xenograft mouse model engrafted with human PBMCs is established as follows: B-NDG B2M KO plus (vendor Biocytogen) mice are subcutaneously transplanted with A375 cells admixed with human PBMCs. The human A375 cells endogenously express human HER3 and human PD-L1. Treatments with the ADC/antibody indicated below are started when the tumour size is around 100 mm³. 20 mg/kg of anti-hPD1xhVEGF (Compound 13) are administered 3 times biweekly. 1 mg/kg of the anti-hHER3-ADC (Compound 4) is administered once. Monotherapy control groups G2, and G3 are treated only either with the anti-hPD1xhVEGF (Compound 13) or the anti-hHER3 ADC compound (Compound 4). The Vehicle control group G1 is treated with PBS only. The study design is summarized in Table XIII.

The combination of the anti-hHER3 ADC and the anti-hPD1xhVEGF is expected to result in tumour regression and tumour control, whereas monotherapy treatments result in shorter-term tumour control and outgrowth of tumours.

TABLE XIII
Study design anti-hHER3 (Compound
4) + anti-hPD1xhVEGF (Compound 6):

| Groups | Treatment | Dose | Dosing Schedule |
|---|---|---|---|
| G1 | Vehicle | — | D 0, D 3, D 7 |
| G2 | Compound 13 | 20 mg/kg | D 0, D 3, D 7 |
| G3 | Compound 4 | 1 mg/kg | D 0 |
| G4 | Compound 13 + | 20 mg/kg and | D 0, D 3, D 7 |
| | Compound 4 | 1 mg/kg | and D 0 |

1-103. (canceled)

104. A method of treating cancer in a subject, the method comprising administering to the subject:
a. a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction; and
b. an antibody-drug conjugate comprising an antibody or fragment thereof that specifically binds to a target protein,
wherein the cancer comprises cells that express the target protein.

105. The method of claim 104, wherein:
(i) the cancer comprises cells that express HER2 and the target protein is HER2, or
(ii) the cancer comprises cells that express B7H3 and the target protein is B7H3, or
(iii) the cancer comprises cells that express TROP2 and the target protein is TROP2, or
(iv) the cancer comprises cells that express HER3 and the target protein is HER3, or
(v) the cancer comprises cells that express CD30 and the target protein is CD30, or
(vi) the cancer comprises cells that express Nectin-4 and the target protein is Nectin-4, or
(vii) the cancer comprises cells that express TF and the target protein is TF, or
(viii) the cancer comprises cells that express B7-H4 and the target protein is B7-H4, or
(ix) the cancer comprises cells that express ITGB6 and the target protein is ITGB6.

106. The method of claim 104, wherein the cancer is a solid tumour.

107. The method of claim 104, wherein the binding agent comprises:
(a) an anti-VEGF antibody and at least one anti-PD-L1 single domain antibody, optionally an anti-VEGF antibody and two anti-PD-L1 single domain antibodies; or
(b) an anti-VEGF antibody and at least one anti-PD-1 scFv antibody, optionally an anti-VEGF antibody and two anti-PD-1 scFv antibodies; or
(c) an anti-VEGF antibody and at least one anti-PD-1 single domain antibody, optionally an anti-VEGF antibody and at least two anti-PD-1 single domain antibodies; or
(d) a fusion protein comprising an anti-PD-L1 antibody and at least one VEGFR domain; or
(e) a first binding region which binds to PD-1 and a second binding region which binds to VEGFR.

108. The method of claim 104, wherein the antibody-drug conjugate comprises:
(a) the antibody or fragment thereof linked to one or more linker-payload moieties; or
(b) the antibody or fragment thereof linked to one or more linker-payload moieties wherein the payload moiety is a cytotoxic drug, optionally wherein the cytotoxic drug is a tubulin polymerisation inhibitor, a DNA damaging agent, a TOPO1 inhibitor, an auristatin, a maytansinoid, or a calicheamicin; or
(c) the antibody or fragment thereof linked to one or more linker-payload moieties wherein the linker is a cleavable linker comprising a peptide linker consisting of 1 to 10 amino acid residues.

109. The method of claim 104, wherein the antibody or fragment thereof of the antibody-drug conjugate comprises a heavy chain variable region and a light chain variable region, each comprising three complementarity determining regions (CDRs), which CDRs provide a means for binding to the target protein.

110. The method of claim 104, wherein at least 10%, at least 20%, at least 30%, at least 40%, or at least 50% of the cancer cells express the target protein.

111. A kit comprising:
(a) a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction, and instructions for administering the binding agent with an antibody-drug conjugate comprising an antibody or fragment thereof that specifically binds to a target protein, for treatment of a cancer that comprises cells that express the target protein; or
(b) an antibody-drug conjugate comprising an antibody or fragment thereof that specifically binds to a target protein, and instructions for administering the antibody-drug conjugate with a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction, for treatment of a cancer that comprises cells that express the target protein.

112. The kit of claim 111, wherein:
(i) the cancer comprises cells that express HER2 and the target protein is HER2, or
(ii) the cancer comprises cells that express B7H3 and the target protein is B7H3, or
(iii) the cancer comprises cells that express TROP2 and the target protein is TROP2, or
(iv) the cancer comprises cells that express HER3 and the target protein is HER3, or
(v) the cancer comprises cells that express CD30 and the target protein is CD30, or
(vi) the cancer comprises cells that express Nectin-4 and the target protein is Nectin-4, or
(vii) the cancer comprises cells that express TF and the target protein is TF, or
(viii) the cancer comprises cells that express B7-H4 and the target protein is B7-H4, or
(ix) the cancer comprises cells that express ITGB6 and the target protein is ITGB6.

113. The kit of claim 111, wherein the binding agent comprises:
(a) an anti-VEGF antibody and at least one anti-PD-L1 single domain antibody, optionally an anti-VEGF antibody and two anti-PD-L1 single domain antibodies;
(b) an anti-VEGF antibody and at least one anti-PD-1 scFv antibody, optionally an anti-VEGF antibody and two anti-PD-1 scFv antibodies;
(c) an anti-VEGF antibody and at least one anti-PD-1 single domain antibody, optionally an anti-VEGF antibody and at least two anti-PD-1 single domain antibodies;
(d) a fusion protein comprising an anti-PD-L1 antibody and at least one VEGFR domain; or
(e) a first binding region which binds to PD-1 and a second binding region which binds to VEGFR.

114. The kit of claim 111, wherein the antibody-drug conjugate comprises:
(a) the antibody or fragment thereof linked to one or more linker-payload moieties; or
(b) the antibody or fragment thereof linked to one or more linker-payload moieties wherein the payload moiety is a cytotoxic drug, optionally wherein the cytotoxic drug is a tubulin polymerisation inhibitor, a DNA damaging agent, a TOPO1 inhibitor, an auristatin, a maytansinoid, or a calicheamicin; or
(c) the antibody or fragment thereof linked to one or more linker-payload moieties wherein the linker is a cleavable linker comprising a peptide linker consisting of 1 to 10 amino acid residues.

115. The kit of claim 111, wherein the antibody or fragment thereof of the antibody-drug conjugate comprises a heavy chain variable region and a light chain variable region, each comprising three complementarity determining regions (CDRs), which CDRs provide a means for binding to the target protein.

116. The kit of claim 111, wherein at least 10%, at least 20%, at least 30%, at least 40%, or at least 50% of the cancer cells express the target protein.

117. A combination comprising
a. a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction; and
b. an antibody-drug conjugate comprising an antibody or fragment thereof that specifically binds to a target protein.

118. The combination of claim 117, wherein the target protein is expressed by a tumor cell.

119. The combination of claim 117, wherein:
(i) the target protein is HER2, or
(ii) the target protein is B7H3, or
(iii) the target protein is TROP2, or
(iv) the target protein is HER3, or
(v) the target protein is CD30, or
(vi) the target protein is Nectin-4, or
(vii) the target protein is TF, or
(viii) the target protein is B7-H4, or
(ix) the target protein is ITGB6.

120. The combination of claim 117, wherein the binding agent comprises:
(a) an anti-VEGF antibody and at least one anti-PD-L1 single domain antibody, optionally an anti-VEGF antibody and two anti-PD-L1 single domain antibodies;
(b) an anti-VEGF antibody and at least one anti-PD-1 scFv antibody, optionally an anti-VEGF antibody and two anti-PD-1 scFv antibodies;
(c) an anti-VEGF antibody and at least one anti-PD-1 single domain antibody, optionally an anti-VEGF antibody and at least two anti-PD-1 single domain antibodies;
(d) a fusion protein comprising an anti-PD-L1 antibody and at least one VEGFR domain; or
(e) a first binding region which binds to PD-1 and a second binding region which binds to VEGFR.

121. The combination of claim 117, wherein the antibody-drug conjugate comprises:
(a) the antibody or fragment thereof linked to one or more linker-payload moieties; or
(b) the antibody or fragment thereof linked to one or more linker-payload moieties wherein the payload moiety is a cytotoxic drug, optionally wherein the cytotoxic drug is a tubulin polymerisation inhibitor, a DNA damaging agent, a TOPO1 inhibitor, an auristatin, a maytansinoid, or a calicheamicin; or
(c) the antibody or fragment thereof linked to one or more linker-payload moieties wherein the linker is a cleavable linker comprising a peptide linker consisting of 1 to 10 amino acid residues.

122. The combination of claim 117, wherein the antibody or fragment thereof of the antibody-drug conjugate comprises a heavy chain variable region and a light chain variable region, each comprising three complementarity determining regions (CDRs), which CDRs provide a means for binding to the target protein.

123. The combination of claim 117, wherein the binding agent and the antibody-drug conjugate are comprised in separate containers.

124. A method of treating cancer in a subject, the method comprising administering to the subject the combination of claim 117, wherein the cancer comprises cells that express the target protein.

125. The method of claim 124, wherein at least 10%, at least 20%, at least 30%, at least 40%, or at least 50% of the cancer cells express the target protein.

126. A method of administering to a subject, the method comprising administering to the subject:
a. a binding agent comprising (i) a first binding region which binds to PD-1, PD-L1, or both and which antagonizes the PD-1/PD-L1 interaction; and (ii) a second binding region which binds to VEGF, VEGF receptor (VEGFR), or both and which antagonizes the VEGF/VEGFR interaction; and
b. an antibody-drug conjugate comprising an antibody or fragment thereof that specifically binds to a target protein.

127. The method of claim 126, wherein:
(i) the target protein is HER2, or
(ii) the target protein is B7H3, or
(iii) the target protein is TROP2, or
(iv) the target protein is HER3, or
(v) the target protein is CD30, or
(vi) the target protein is Nectin-4, or
(vii) the target protein is TF, or
(viii) the target protein is B7-H4, or
(ix) the target protein is ITGB6.

128. The method of claim 126, wherein the binding agent comprises:
(a) an anti-VEGF antibody and at least one anti-PD-L1 single domain antibody, optionally an anti-VEGF antibody and two anti-PD-L1 single domain antibodies;
(b) an anti-VEGF antibody and at least one anti-PD-1 scFv antibody, optionally an anti-VEGF antibody and two anti-PD-1 scFv antibodies;
(c) an anti-VEGF antibody and at least one anti-PD-1 single domain antibody, optionally an anti-VEGF antibody and at least two anti-PD-1 single domain antibodies;
(d) a fusion protein comprising an anti-PD-L1 antibody and at least one VEGFR domain; or
(e) a first binding region which binds to PD-1 and a second binding region which binds to VEGFR.

129. The method of claim 126, wherein the antibody-drug conjugate comprises:
(a) the antibody or fragment thereof linked to one or more linker-payload moieties; or
(b) the antibody or fragment thereof linked to one or more linker-payload moieties wherein the payload moiety is a cytotoxic drug, optionally wherein the cytotoxic drug is a tubulin polymerisation inhibitor, a DNA damaging agent, a TOPO1 inhibitor, an auristatin, a maytansinoid, or a calicheamicin; or
(c) the antibody or fragment thereof linked to one or more linker-payload moieties wherein the linker is a cleavable linker comprising a peptide linker consisting of 1 to 10 amino acid residues.

130. The method of claim 126, wherein the antibody or fragment thereof of the antibody-drug conjugate comprises a heavy chain variable region and a light chain variable region, each comprising three complementarity determining regions (CDRs), which CDRs provide a means for binding to the target protein.

131. The method of claim 126, wherein the binding agent and the antibody-drug conjugate are administered separately.

132. The method of claim 126, wherein the administering has the effect in the subject of slowing growth of cancer cells, decreasing the viability of cancer cells, or inhibiting tumor growth.

133. The method of claim 132, wherein at least 10%, at least 20%, at least 30%, at least 40%, or at least 50% of the cancer cells or cells of the tumor express the target protein.
